(19) United States
(12) Patent Application Publication
(10) Pub. No.: US 2025/0340623 A1
(43) Pub. Date: Nov. 6, 2025

(54) Novel Molecules for Therapy and Diagnosis

(71) Applicant: AC Immune SA, Lausanne (CH)

(72) Inventors: Elpida Tsika, Lausanne (CH); John Warner, Lausanne (CH); Romain Christian Ollier, Lausanne (CH); Jan Peter Henning Stöhr, Lausanne (CH); Marie Kosco-Vilbois, Lausanne (CH)

(21) Appl. No.: 17/782,536

(22) PCT Filed: Dec. 4, 2020
(86) PCT No.: PCT/EP2020/084776

§ 371 Date: Jun. 3, 2022

(30) Foreign Application Priority Data
Dec. 4, 2019 (EP) 19213703.2
Oct. 16, 2020 (EP) 20202412.1

(51) Int. Cl. C07K 16/18; A61K 47/68; G01N 33/68

(52) U.S. Cl. CPC C07K 16/18; A61K 47/6811; G01N 33/6896; C07K 2317/31; C07K 2317/34; C07K 2317/55; C07K 2317/76; C07K 2317/92; G01N 2800/52

(57) ABSTRACT

The present invention relates to biparatopic antigen-binding molecules, such as biparatopic antibodies or functional fragments thereof, and mixtures comprising at least two monospecific antibodies or functional fragments thereof, that can be employed for the prevention, alleviation, treatment and/or diagnosis of diseases, disorders and abnormalities associated with CNS proteins such as alpha-synuclein (α-synuclein, A-synuclein, aSynuclein, A-syn, α-syn, aSyn, a-syn), Tau, TDP-43, ASC, NLRP3, C5a, C1q, C3, huntingtin or prion protein. The present invention further relates to the use of the molecules of the invention for determining a pre-disposition to a disorder, disease or abnormality, monitoring residual disorder, disease or abnormality associated with CNS proteins such as alpha-synuclein (α-synuclein, A-synuclein, aSynuclein, A-syn, α-syn, aSyn, a-syn), Tau, TDP-43, ASC, NLRP3, C5a, C1q, C3, huntingtin or prion protein, or predicting the responsiveness of a patient who is suffering from such a disorder, disease or abnormality to the treatment with a certain medicament.

### INCORPORATION BY REFERENCE OF SEQUENCE LISTING PROVIDED AS A TEXT FILE

A Sequence Listing is provided herewith in a text file, BOULT-044_SEQ_LIST_ST25, created on Oct. 31, 2022 and having a size of 394,894 bytes. The contents of the text file are incorporated herein by reference in its entirety.

### FIELD OF THE INVENTION

The present invention relates to biparatopic antigen-binding molecules, such as biparatopic antibodies (bAbs) or functional fragments thereof, and mixtures comprising at least two monospecific antibodies or functional fragments thereof, that can be employed for the prevention, alleviation, treatment and/or diagnosis of diseases, disorders and abnormalities associated with CNS proteins such as alpha-synuclein (α-synuclein, A-synuclein, aSynuclein, A-syn, α-syn, aSyn, a-syn), Tau, TAR DNA-binding protein 43 (TDP-43), Apoptosis-associated speck-like protein containing a CARD (ASC), NACHT, LRR and PYD domains-containing protein 3 (NLRP3), Complement component 5a (C5a), Complement component 1q (C1q), Complement component 3 (C3), huntingtin (Htt) or prion protein.

The present invention further relates to the use of the molecules of the invention for determining a predisposition to a disorder, disease or abnormality, monitoring residual disorder, disease or abnormality associated with CNS proteins such as alpha-synuclein (α-synuclein, A-synuclein, aSynuclein, A-syn, α-syn, aSyn, a-syn), Tau, TDP-43, ASC, NLRP3, C5a, C1q, C3, huntingtin or prion protein, or predicting the responsiveness of a patient who is suffering from such a disorder, disease or abnormality to the treatment with a certain medicament.

### BACKGROUND OF THE INVENTION

There are several ways to develop combinatorial immunotherapies, one could use monospecific antibodies and administer them in combination or as antibody mixtures (Poulsen et al., Clin. Cancer Res. 2017 23(19):5923-5935), however the cost associated with the development of combinatorial immunotherapies is increased compared to traditional/standard monoclonal antibody therapies.

Recent advances in antibody technologies led to the development of bispecific antibodies which are recombinant antibodies that consist of two distinct binding domains capable of binding two different antigens or two different epitopes of the same antigen. Dual-targeting concepts enabled by bispecific antibodies hold good therapeutic promise to deliver enhanced efficacy and/or new mechanism of action. Since the early 2000s, the interest in bispecific antibodies field has grown, with over 100 bispecific formats known today (Brinkmann and Kontermann. Mabs, 2017; 9(2): 182-212). Overcoming heavy and light chains mispairing generated by the concomitant expression of four different chains is the main challenge for bispecific antibody design. Many solutions and formats can be used to produce the correct chain assembly and enable dual binding. The so-called BiTE molecule (Baeuerle et al., 2009 Cancer Res.; 69(12):4941-4) uses flexible linkers to fuse VH and VL domains to enable the correct VH/VL pairing, yet this format lacks the Fc domain. To maintain antibody properties such as Fc mediated effector functions, long serum half-life or stability, others have developed IgG-like molecules by genetically engineering antibody constant domains to eliminate the unwanted species made by the random assembly of heavy and light chain. Correct heavy chain pairing can be mediated by CH3 heterodimerization using the knobs-into-holes technology (Ridgway et al., Protein Engineering, 1996 9 (7) 617-621). A different approach was described by Smith et al (Sci Rep.; 2015, 5:17943) where the correct pair of heavy chain is isolated by modifying the binding to protein A of one heavy chain. Recently, Fischer et al (Nat Commun. 2015; 6:6113) have used a common heavy chain phage libraries combined with kappa and lambda light chains to generate full IgG molecules. The Duobody® technology utilizes the natural phenomenon of Fab arm exchange to assemble bispecific antibodies in vitro using mild reducing agents (Labrjin et al, PNAS, 2013 110 (13) 5145-5150).

To overcome the wrong assembly of heavy and light chains, also called light chain mispairing, some have used a common light chain phage libraries (Merchant et al., Nat Biotechnol.; 1998 16(7):677-81) whereas others developed formats containing an scFv on one of the binding arm (Skegro et al., JBC; 2018, 292(23) 9745-9759). Several approaches have modified the natural constant (including CH1-CL) domains to enable the correct formation of the bispecific antibody arms. Schaefer et al., PNAS, 2011, 108 (27) 11187-11192 described the exchange of CH1 and CL domain to correctly assemble heavy and light chains. The natural TCR α/β heterodimers were also used to replace the CH1/CL domains and produce IgG-like molecules (Wu et al., Mabs, 2015, 7(2), 364-376 and WO2019/057122 Others also used artificially introduced disulfide bond in the heavy and light chain constant domain to enhance cognate chain assembly (Mazor et al, mAbs, 2015, 7(2): 377-389.)) Labrjn et al, PNAS, 2013 110 (13) 5145-5150 describe a process that involves separate expression of two parental antibodies, each containing single matched point mutations in the CH3 domains. The parental antibodies are mixed and subjected to controlled reducing conditions in vitro that separate the antibodies into HL half-molecules and allow reassembly and reoxidation to form highly pure bsAbs.

There are over 85 bispecific antibodies in clinical development and many more in preclinical testing, (Labrjin et al., Nat Rev Drug Discov., 2019). The variety of formats in the bispecific antibody landscape shows that one can modify the valency or the geometry. Antibody fragments such as scFv, Fabs or VHH can be recombinantly fused to the bispecific antibody creating so-called 1+2 and 2+2 molecules as opposed to the 1+1 bispecific antibody.

The vast majority of the bispecific antibodies in development are designed for cancer therapies. To date, only a few bispecific antibodies have been designed for the treatment of central nervous system (CNS) diseases, mainly targeting a protein associated with CNS diseases and a receptor to mediate the antibody's transcytosis across the blood-brain-barrier. To our knowledge, there is no bispecific or biparatopic antibody in clinical development targeting alpha-synuclein protein. To our knowledge, the biparatopic antibody or a functional fragment thereof targeting alpha-synuclein protein are made and described for the first time in the present invention.

Alpha-synuclein is a 140 amino acid long, cytosolic protein abundantly and predominantly expressed in the CNS and localized in pre-synaptic terminals (Burré J., J Parkinsons Dis. 2015; 5(4):699-713). Alpha-synuclein is a natively unfolded protein but adopts secondary structure of mostly helical nature upon association with lipid vesicles or membranes (Iwai et al., Biochemistry 1995, 34(32), 10139-10145). The physiological function of alpha-synuclein remains elusive. Because of the association of alpha-synuclein with synaptic vesicles and its presynaptic localization it is suggested that it regulates synaptic activity and plasticity, neurotransmitter release, dopamine production and metabolism, vesicle trafficking, synaptic vesicle pool maintenance and might also exhibit chaperone-like activity (Cabin et al., J Neurosci. 2002; 22:8797-8807; Chandra et al., Cell. 2005; 123:383-396).

To date the molecular mechanism underlying alpha-synuclein aggregation and spreading in synucleinopathies remains elusive and the role of the different sequence segments/domains of alpha-synuclein in this process is poorly understood. The sequence of alpha-synuclein can be divided into three main domains: 1) the N-terminal region comprising of residues 1-60, which contains 11-mer amphipatic imperfect repeat residues with highly conserved hexameric sequence (KTKEGV). This region has been implicated in regulating alpha-synuclein association to lipid membranes and its internalization. It also bears all the genetic mutations identified to date which are associated with familial Parkinson's Disease (PD); 2) the hydrophobic Non-Amyloid beta Component (NAC) domain spanning residues 61-95 folds into a s-sheet secondary structure and plays a critical role in both aggregation and cytotoxicity; and 3) the C-terminal region spanning residues 96-140 which is structurally dynamic because of its low hydrophobicity and high net negative charge. Most of the alpha-synuclein antibodies aiming to prevent the cellular spreading of alpha-synuclein aggregation target the C-terminal region (Zella et al., Neurol Ther. 2019; 8(1):29-44).

Alpha-synuclein is able to transition between different conformations, such as monomers, oligomers or fibrils and aggregates, yet the pathological form(s) of alpha-synuclein remain ambiguous. The concept of multiple strains or variants of pathological protein aggregates existing in a “cloud of conformations” was first described for prion proteins (Bateman et al., PLoS Genet. 2013; 9(1)) and has since been described for other amyloidogenic proteins including, but not limited to, Amyloid beta (Rasmussen et al., Proc Natl Acad Sci USA. 2017; 114(49):13018-13023) and alpha-synuclein (Jucker et al., Nat Neurosci. 2018; 21(10):1341-1349). The heterogeneity of the targeted species as well as the aggregation process which involves all domains of alpha-synuclein is highly challenging for the development of immunotherapies.

### SUMMARY OF THE INVENTION

It is an object of the present invention to provide improved antigen-binding molecules that target CNS proteins that can be employed to treat, alleviate and/or prevent a disease, disorder or abnormality (also referred to herein as a condition) associated with the CNS proteins.

It is an object of the present invention to provide improved alpha-synuclein antigen-binding molecules that can be employed to treat, alleviate and/or prevent a disease, disorder or abnormality (also referred to herein as a condition) associated with alpha-synuclein aggregates.

It is also an object of the present invention to provide improved antigen-binding molecules that target CNS proteins that can be employed to diagnose, monitor disease progression of, and/or monitor drug activity against, a disease, disorder or abnormality associated with the CNS proteins.

It is also an object of the present invention to provide improved antigen-binding molecules that can be employed to diagnose, monitor disease progression of, and/or monitor drug activity against, a disease, disorder or abnormality (also referred to herein as a condition) associated with alpha-synuclein aggregates.

The technical problem is solved by the embodiments provided herein and as characterized in the claims. Accordingly, the invention relates to a biparatopic antibody or functional fragment thereof which binds at least two distinct epitopes of a protein associated with a CNS disease, such as alpha-synuclein, Tau, TAR DNA-binding protein 43 (TDP-43), Apoptosis-associated speck-like protein containing a CARD (ASC), NACHT, LRR and PYD domains-containing protein 3 (NLRP3), Complement component 5a (C5a), Complement component 1q (C1q), Complement component 3 (C3), huntingtin or prion protein.

Accordingly, the invention, in a first aspect, relates to an alpha-synuclein biparatopic antibody or functional fragment thereof which binds at least two distinct epitopes of alpha-synuclein, preferably human alpha-synuclein (having the amino acid sequence) of SEQ ID NO: 1.

It was surprisingly found that biparatopic antigen-binding molecules targeting simultaneously distinct epitopes on a protein associated with a CNS disease, such as alpha-synuclein or any one of Tau, TAR DNA-binding protein 43 (TDP-43), Apoptosis-associated speck-like protein containing a CARD (ASC), NACHT, LRR and PYD domains-containing protein 3 (NLRP3), Complement component 5a (C5a), Complement component 1q (C1q), Complement component 3 (C3), huntingtin or prion protein, represent a compelling approach to enhance the therapeutic effect of standard monospecific monoclonal antibody therapies and trigger new therapeutic mechanism of action. The biparatopic antigen-binding molecules of the invention comprise biparatopic antibodies or functional fragments thereof, mixtures of monospecific monoclonal antibodies or functional fragments thereof, mixtures of biparatopic antibodies or functional fragments thereof and at least one monospecific monoclonal antibody or a functional fragment thereof, amongst others.

The present invention describes in particular the use of biparatopic antigen-binding molecules, such as biparatopic antibodies or functional fragments thereof, and mixtures comprising at least two monospecific antibodies able to simultaneously recognize two distinct epitopes on a protein associated with a CNS disease, such as alpha-synuclein (i.e. biparatopic) or Tau, TAR DNA-binding protein 43 (TDP-43), Apoptosis-associated speck-like protein containing a CARD (ASC), NACHT, LRR and PYD domains-containing protein 3 (NLRP3), Complement component 5a (C5a), Complement component 1q (C1q), Complement component 3 (C3), huntingtin or prion protein. Surprisingly, the use of binding molecules to a protein associated with a CNS disease, in particular the alpha-synuclein binding molecules, in combination (i.e. polypeptide complex made of two polypeptides) described herein showed a synergistic effect at inhibiting and/or delaying seeded and/or spontaneous aggregation of the protein associated with a CNS disease, in particular alpha-synuclein aggregation, and thus yielded better inhibition/delaying aggregation efficacy than the individual, monospecific binding molecule, in particular the alpha-synuclein binding molecules, tested separately. For example some biparatopic binding molecules described herein simultaneously recognize an epitope at the NAC domain and another in the C-terminus of alpha-synuclein offering the advantage of binding to a broader range of alpha-synuclein species including C-terminally truncated alpha-synuclein aggregates. Similarly, the binding to two distinct epitopes on a protein associated with a CNS disease other than alpha-synuclein allows binding to various species thereof, thereby allowing to target a broader range of protein species. The present invention also describes the use of a combination of two monospecific binding molecules to a protein associated with a CNS disease, in particular alpha-synuclein binding molecules, in a mixture able to simultaneously recognize two distinct epitopes on the protein associated with a CNS disease, in particular alpha synuclein. In addition, the present invention also describes the use of a combination of binding molecules to a protein associated with a CNS disease, in particular alpha-synuclein binding molecules, in a mixture comprising biparatopic antibodies or functional fragments thereof able to simultaneously recognize two distinct epitopes on the protein associated with a CNS disease, in particular alpha-synuclein and a monospecific antibody or functional fragment thereof which target one epitope on the protein associated with a CNS disease, in particular alpha-synuclein.

The disease, disorder and/or abnormality (also referred to herein as a condition) associated with alpha-synuclein aggregate may be a synucleinopathy. In some embodiments, the synucleinopathy is Parkinson's disease (sporadic, familial with alpha-synuclein mutations, familial with mutations other than alpha-synuclein, pure autonomic failure and Lewy body dysphagia), Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

Particularly, the synucleinopathy may be selected from Parkinson's Disease, Multiple System Atrophy, Lewy Body dementia (LBD; including dementia with Lewy bodies (DLB) (“pure” Lewy body dementia), Parkinson's disease dementia (PDD)), or Diffuse Lewy Body Disease.

Accordingly, the invention relates in its broadest aspect to a biparatopic antibody or functional fragment thereof which binds at least two distinct epitopes of a protein associated with a CNS disease, such as alpha-synuclein, Tau, TDP-43, ASC, NLRP3, C5a, C1q, C3, huntingtin or prion protein, and mixtures comprising at least two monospecific antibodies or functional fragments thereof, wherein both monospecific antibodies or functional fragments thereof bind the same protein associated with a CNS disease but distinct epitopes, such as alpha-synuclein, Tau, TDP-43, ASC, NLRP3, C5a, C1q, C3, huntingtin or prion protein, mixtures of biparatopic antibodies or functional fragments thereof and at least one monospecific monoclonal antibody or a functional fragment thereof, amongst others. Provided are biparatopic antigen-binding molecules targeting alpha-synuclein, in particular biparatopic antibodies or functional fragments thereof, and mixtures comprising at least two monospecific antibodies or functional fragments thereof, mixtures of biparatopic antibodies or functional fragments thereof and at least one monospecific monoclonal antibody or a functional fragment thereof, amongst others. In one embodiment, the invention relates to a biparatopic antibody or functional fragment thereof which binds at least two distinct epitopes of a protein associated with a CNS disease, such as alpha-synuclein, Tau, TDP-43, ASC, NLRP3, C5a, C1q, C3, huntingtin or prion protein, in particular an alpha-synuclein biparatopic binding molecule, which inhibits and/or delays seeded and/or spontaneous aggregation of the protein associated with a CNS disease, such as alpha-synuclein, Tau, TDP-43, ASC, NLRP3, C5a, C1q, C3, huntingtin or prion protein, in particular alpha-synuclein aggregation. In a particular embodiment of the invention, the biparatopic antigen-binding molecules targeting a protein associated with a CNS disease, such as alpha-synuclein, Tau, TDP-43, ASC, NLRP3, C5a, C1q, C3, huntingtin or prion protein, in particular alpha-synuclein, in particular biparatopic antibodies or functional fragments thereof, and mixtures comprising at least two monospecific antibodies or functional fragments thereof, mixtures of biparatopic antibodies or functional fragments thereof and at least one monospecific monoclonal antibody or a functional fragment thereof, inhibit and/or delay the aggregation of seeded and/or spontaneous aggregation of the protein associated with a CNS disease, such as alpha-synuclein, Tau, TDP-43, ASC, NLRP3, C5a, C1q, C3, huntingtin or prion protein, in particular alpha-synuclein aggregation. In another embodiment of the invention, the biparatopic antigen-binding molecules targeting a protein associated with a CNS disease, such as alpha-synuclein, Tau, TDP-43, ASC, NLRP3, C5a, C1q, C3, huntingtin or prion protein, in particular alpha-synuclein, in particular biparatopic antibodies or functional fragments thereof, and mixtures comprising at least two monospecific antibodies or functional fragments thereof, mixtures of biparatopic antibodies or functional fragments thereof and at least one monospecific monoclonal antibody or a functional fragment thereof, are capable of recognizing and binding to pathological or aggregated protein associated with a CNS disease, such as alpha-synuclein, Tau, TDP-43, ASC, NLRP3, C5a, C1q, C3, huntingtin or prion protein, in particular alpha-synuclein, particularly human alpha-synuclein, in vitro and in vivo.

Within the scope of the present invention, alpha-synuclein may have the sequence of SEQ ID NO: 1. Alpha-synuclein aggregates are multimeric beta-sheet rich assemblies of alpha-synuclein monomers that can form either soluble oligomers or soluble/insoluble protofibrils or mature fibrils which coalesce into intracellular deposits detected as a range of Lewy pathologies in Parkinson's disease and other synucleinopathies. Alpha-synuclein under physiological conditions does not adopt an ordered tertiary structure, rather it is classified as a natively unfolded protein which can exist as a mixture of dynamic and flexible structural conformations.

Misfolded alpha-synuclein can form multimeric intermediate oligomeric structures which eventually assemble into highly-ordered fibrillar aggregates.

Pathological alpha-synuclein may be misfolded or aggregated or post-translationally modified alpha-synuclein that is the main component of Lewy pathologies; Lewy pathologies can be detected as having the following morphologies: Lewy bodies, Lewy neurites, premature Lewy bodies or pale bodies, perikaryal deposits with diffuse, granular, punctate or pleomorphic patterns.

Pathological alpha-synuclein can exist in multiple conformations between distinct synucleinopathies and within a specific synucleinopathy.

Lewy bodies are abnormal aggregates of protein that develop inside nerve cells in Parkinson's disease (PD), Lewy body dementia and other synucleinopathies. Lewy bodies appear as spherical masses that displace other cell components. Morphologically, Lewy bodies can be classified as being brainstem or cortical type. Classic brainstem Lewy bodies are eosinophilic cytoplasmic inclusions consisting of a dense core surrounded by a halo of 5-10-nm-wide radiating fibrils, the primary structural component of which is alpha-synuclein; cortical Lewy bodies differ by lacking a halo. The presence of Lewy bodies is a hallmark of Parkinson's disease.

Lewy neurites are abnormal neuronal processes in diseased neurons, containing granular material, abnormal alpha-synuclein filaments similar to those found in Lewy bodies, dot-like, varicose structures and axonal spheroids. Like Lewy bodies, Lewy neurites are a feature of a-synucleinopathies such as Lewy Body dementia (LBD; including dementia with Lewy bodies (DLB) (“pure” Lewy body dementia), Parkinson's disease dementia (PDD)), or Diffuse Lewy Body Disease, Parkinson's disease, and multiple system atrophy.

Glial cytoplasmic inclusions (also referred to as Papp-Lantos inclusions) consist of insoluble alpha-synuclein filamentous aggregates detected in oligodendrocytes in the white matter of multiple system atrophy brains. Alpha-synuclein aggregates in neuronal somata, axons and nuclei, referred to as neuronal cytoplasmic inclusions, are characteristic cytopathological features of multiple system atrophy. The detection of glial cytoplasmic inclusions is considered hallmark for the neuropathological diagnosis of multiple system atrophy.

Moreover, pathological alpha-synuclein is the major component of intracellular fibrillary inclusions detected in oligodendrocytes also referred to as glial cytoplasmic inclusions and in neuronal somata, axons and nuclei (referred to as neuronal cytoplasmic inclusions) that are the histological hallmarks of multiple system atrophy. Pathological alpha-synuclein in Lewy pathologies often displays substantial increase in post-translational modifications such as phosphorylation, ubiquitination, nitration, and truncation.

Alpha-synuclein is an intrinsically disordered protein, which has the propensity to spontaneously aggregate and form soluble oligomers or soluble/insoluble protofibrils or mature fibrils or detergent-insoluble aggregates under certain conditions. Aggregates of alpha-synuclein can act as seeds thereby recruiting and converting native alpha-synuclein monomers into the fibril state, a process known as seeding (Wood et al., J Biol Chem. 1999 Jul. 9; 274(28):19509-12).

Seeds are multimeric beta-sheet rich structures which are composed of alpha-synuclein or could be also composed of other amyloidogenic proteins (e.g. Tau, Amyloid s) which can accelerate the aggregation kinetics of alpha-synuclein by elongating the growing multimer and/or by acting as templates for the nucleation of monomers on the seed surface.

Spontaneous aggregation of alpha-synuclein is the aggregation process that progresses without the addition of seeds. Alpha-synuclein is a soluble protein that has the propensity to spontaneously aggregate and form soluble oligomers or soluble/insoluble protofibrils or mature fibrils or detergent-insoluble aggregates under certain conditions.

Recent evidence from cellular and animal models suggests that pathological or aggregated alpha-synuclein can spread from one neuron to another. Once inside the new cell alpha-synuclein aggregates act as seeds, recruiting endogenous alpha-synuclein and advancing protein aggregation (Luk et al., Science. 2012, 338(6109):949-5; Tran et al., Cell Rep. 2014, 7(6):2054-65). Moreover, the transsynaptic spreading of pathological or aggregated alpha-synuclein could explain the progressive advancing of Lewy pathology through defined anatomical connected brain areas in PD that was first described by Braak and colleagues (Braak et al., Neurobiol. Aging. 2003; 24:197-211). Aggregation and spreading of alpha-synuclein through different brain structures is contributing to multiple neurodegenerative diseases known as synucleinopathies, including but not limited to, Parkinson's disease (PD), Lewy Body dementia (LBD; including dementia with Lewy bodies (DLB) (“pure” Lewy body dementia), Parkinson's disease dementia (PDD)) or Diffuse Lewy Body Disease, and multiple system atrophy (MSA) (Jellinger, Mov Disord 2003, 18 Suppl. 6, S2-12). Different alpha-synuclein aggregate species have shown distinct seeding capacities in vitro and in vivo.

Seeded alpha-synuclein aggregation is the aggregation accelerated by pathological alpha-synuclein, so-called “seeds”.

The biparatopic antigen-binding molecules of the invention that bind a protein associated with a CNS disease, such as alpha-synuclein, Tau, TDP-43, ASC, NLRP3, C5a, C1q, C3, huntingtin or prion protein, in particular the alpha-synuclein biparatopic antigen-binding molecules of the invention, in particular biparatopic antigen-binding molecules targeting alpha-synuclein, in particular biparatopic antibodies or functional fragments thereof, and mixtures comprising at least two monospecific antibodies or functional fragments thereof, and mixtures of biparatopic antibodies or functional fragments thereof and at least one monospecific monoclonal antibody or a functional fragment thereof, have at least one, preferably two, even more preferably all three of the following characteristics:
block cell-to-cell spreading,
capable of recognizing and binding to pathological or aggregated protein associated with a CNS disease, such as alpha-synuclein, Tau, TDP-43, ASC, NLRP3, C5a, C1q, C3, huntingtin or prion protein, in particular alpha-synuclein, particularly human alpha-synuclein,
delay and/or inhibit the aggregation of the protein associated with a CNS disease, such as alpha-synuclein, Tau, TDP-43, ASC, NLRP3, C5a, C1q, C3, huntingtin or prion protein, in particular alpha-synuclein protein or fragments thereof.

As such, the term “functional fragment” as used herein relates to a fragment of the biparatopic antigen-binding molecules of the invention which essentially maintains the functions, or functionality, of the full-length parent molecule, e.g. the functions, or characteristics, defined immediately above. The functional fragment can be defined as the pair of VH/VL of the parental antibody (also referred as “Arms”, such that a functional fragment of the biparatopic antigen-binding molecules of the invention comprises at least distinct pairs or arms of VH/VL). The functional fragment can be further reduced to the paratope of the antibody, i.e. the residues making contact with the antigen. The paratropic residues may be identified by mutation analysis or based on structural analysis of the binding site, such as e.g. analysis based on X-ray crystallographie, NMR, in silico modeling. The parent antigen binding molecule may then be shortened to those sequences required to maintain binding and functionality. Such functional fragments are also encompassed by the present invention. An exemplary functional fragment within the scope of the present invention is a peptidomimetic of a biparatopic antigen-binding molecule, in particular an antibody, provided herein. Such a peptidomimetic may preferably comprise the CDR3 sequences of the heavy chain of the parent antibody.

In particular biparatopic antigen-binding molecules targeting a protein associated with a CNS disease, such as alpha-synuclein, Tau, TDP-43, ASC, NLRP3, C5a, C1q, C3, huntingtin or prion protein, in particular alpha-synuclein, in particular biparatopic antibodies or functional fragments thereof, and mixtures comprising at least two monospecific antibodies or functional fragments thereof, mixtures of biparatopic antibodies or functional fragments thereof and at least one monospecific monoclonal antibody or a functional fragment thereof, inhibit and/or delay aggregation of the protein associated with a CNS disease, such as alpha-synuclein, Tau, TDP-43, ASC, NLRP3, C5a, C1q, C3, huntingtin or prion protein, in particular alpha-synuclein protein or fragments thereof.

In some embodiments, the biparatopic antibody or functional fragment thereof is a murine, murinized, human, humanized, or chimeric biparatopic antibody.

In some embodiments, the biparatopic antibody or functional fragment thereof is fused to a polypeptide binding to a blood-brain barrier receptor such as a receptor transfer unit, a transferrin receptor, an insulin receptor or a low-density lipoprotein receptor. The polypeptide can be a peptide, a single domain antibody (VHH), a scFv or a Fab fragment.

An alpha-synuclein biparatopic antigen-binding molecule is a molecule that preferably binds to the pathological or aggregated alpha-synuclein protein, such as an alpha-synuclein biparatopic antibody or fragment thereof, and simultaneously binds at least two distinct specific recognition sites (epitopes). Some biparatopic antigen-binding molecules of the invention bind to at least two epitopes within the amino acid sequence of SEQ ID NO: 1. Each epitope may be a linear epitope or a non-linear epitope. This discussion applies mutatis mutandis to mixtures of two monospecific antibodies or functional fragments thereof.

Some biparatopic antigen binding molecules of the invention, in particular biparatopic antigen-binding molecules targeting alpha-synuclein, in particular biparatopic antibodies or functional fragments thereof, and mixtures comprising at least two monospecific antibodies or functional fragments thereof, mixtures of biparatopic antibodies or functional fragments thereof and at least one monospecific monoclonal antibody or a functional fragment thereof, bind to at least two epitopes, within amino acids residues 1-60 (N-terminus domain), 60-95 (NAC domain), or 96-140 (C-terminus domain) of human alpha-synuclein of SEQ ID NO: 1. In another embodiment, biparatopic binding molecules of the invention bind to a non-linear epitope within amino acid residues of human alpha-synuclein of SEQ ID NO: 1.

In a particular embodiment, biparatopic antigen-binding molecules of the invention, in particular biparatopic antibodies or functional fragments thereof bind to at least one epitope within amino acids residues 96-140 (C-terminus domain) of human alpha-synuclein of SEQ ID NO: 1.

In another embodiment, biparatopic antigen binding molecules of the invention, in particular biparatopic antibodies or functional fragments thereof bind to a first epitope within amino acids residues 96-140 (C-terminus domain) of human alpha-synuclein of SEQ ID NO: 1 and to a second epitope within the amino acid sequence of SEQ ID NO: 1. In another embodiment, biparatopic binding molecules of the invention, in particular biparatopic antibodies or functional fragments thereof bind to a first epitope within amino acids residues 96-140 (C-terminus domain) of human alpha-synuclein of SEQ ID NO: 1 and to a second epitope within amino acids residues 1-15 (SEQ ID NO: 121), 10-24 (SEQ ID NO: 122), 15-45 (SEQ ID NO: 138), 19-33 (SEQ ID NO: 123), 28-50 (SEQ ID NO: 139), 28-42 (SEQ ID NO: 124), 31-60 (SEQ ID NO: 146), 36-40 (SEQ ID NO: 2), 37-51 (SEQ ID NO: 125), 51-57 (SEQ ID NO: 3), 51-58 (SEQ ID NO: 136), 65-74 (SEQ ID NO: 4), 65-81 (SEQ ID NO: 5), 81-120 (SEQ ID NO: 137), 82-96 (SEQ ID NO: 130), 91-105 (SEQ ID NO: 131), 93-95 (GFV), 96-140 (SEQ ID NO: 147), 100-114 (SEQ ID NO: 132), 109-123 (SEQ ID NO: 133), 118-132 (SEQ ID NO: 134), 124-131 (SEQ ID NO: 7), 127-140 (SEQ ID NO: 135), 128-135 (SEQ ID NO: 8) or 131-140 (SEQ ID NO: 9) of human alpha-synuclein of SEQ ID NO: 1.

In a particular embodiment, biparatopic binding molecules of the invention, in particular biparatopic antibodies or functional fragments thereof bind to at least a first epitope within amino acids residues 96-140 (C-terminus domain) of human alpha-synuclein of SEQ ID NO: 1 and to a second epitope within amino acids residues 96-140 (C-terminus domain) of human alpha-synuclein of SEQ ID NO: 1.

In some embodiments biparatopic antigen-binding molecules of the invention in particular biparatopic antibodies or functional fragments thereof bind to at least one epitope, or at least two distinct epitopes selected from the group of epitopes within amino acids residues 1-15 (SEQ ID NO: 121), 10-24 (SEQ ID NO: 122), 15-45 (SEQ ID NO: 138), 19-33 (SEQ ID NO: 123), 28-50 (SEQ ID NO: 139), 28-42 (SEQ ID NO: 124), 31-60 (SEQ ID NO: 146), 36-40 (SEQ ID NO: 2), 37-51 (SEQ ID NO: 125), 51-57 (SEQ ID NO: 3), 51-58 (SEQ ID NO: 136), 65-74 (SEQ ID NO: 4), 65-81 (SEQ ID NO: 5), 81-120 (SEQ ID NO: 137), 82-96 (SEQ ID NO: 130), 91-105 (SEQ ID NO: 131), 93-95 (GFV), 96-140 (SEQ ID NO: 147), 100-114 (SEQ ID NO: 132), 109-123 (SEQ ID NO: 133), 118-132 (SEQ ID NO: 134), 124-131 (SEQ ID NO: 7), 127-140 (SEQ ID NO: 135), 128-135 (SEQ ID NO: 8) or 131-140 (SEQ ID NO: 9) of human alpha-synuclein of SEQ ID NO: 1.

In some embodiments, an alpha-synuclein biparatopic binding molecule of the invention, in particular a biparatopic antibody or functional fragment thereof according to the invention, comprises a first binding site which binds to a first epitope situated within amino acid residues 65-74 (SEQ ID NO: 4), or 124-131 (SEQ ID NO: 7), or 128-135 (SEQ ID NO: 8), or 131-140 (SEQ ID NO: 9) of human alpha-synuclein of SEQ ID NO: 1 and a second binding site which binds to a second distinct epitope within human alpha-synuclein of SEQ ID NO: 1.

In another embodiment, biparatopic antigen-binding molecules of the invention in particular biparatopic antibodies or functional fragments thereof bind to at least one epitope, or at least two distinct epitopes selected from the group of epitopes within amino acids residues 1-15 (SEQ ID NO: 121), 10-24 (SEQ ID NO: 122), 28-42 (SEQ ID NO: 124), 37-51 (SEQ ID NO: 125), 28-50 (SEQ ID NO: 139), 65-74 (SEQ ID NO: 4), 81-120 (SEQ ID NO: 137), 82-96 (SEQ ID NO: 130), 91-105 (SEQ ID NO: 131), 100-114 (SEQ ID NO: 132), 109-123 (SEQ ID NO: 133), 124-131 (SEQ ID NO: 7), 128-135 (SEQ ID NO: 8) or 131-140 (SEQ ID NO: 9) of human alpha-synuclein of SEQ ID NO: 1. More particularly, biparatopic binding molecules of the invention in particular biparatopic antibodies or functional fragments thereof bind to at least one epitope selected from the group of amino acids residues 124-131 (SEQ ID NO: 7), or 128-135 (SEQ ID NO: 8) or 131-140 (SEQ ID NO: 9) of human alpha-synuclein of SEQ ID NO: 1. In another embodiment, the alpha-synuclein biparatopic binding molecule of the invention in particular biparatopic antibodies or functional fragments thereof binds to two epitopes, one within amino acids 65-74 (SEQ ID NO: 4) and one within amino acids 124-131 (SEQ ID NO: 7); or one within amino acids 124-131 (SEQ ID NO: 7) and one within amino acids 131-140 (SEQ ID NO: 9); or one within amino acids 128-135 (SEQ ID NO: 8) and one within amino acids 124-131 (SEQ ID NO: 7); or one within amino acids 65-74 (SEQ ID NO: 4) and one within amino acids 128-135 (SEQ ID NO: 8); or one within amino acids 65-74 (SEQ ID NO: 4) and one within amino acids 131-140 (SEQ ID NO: 9); or one within amino acids 10-24 (SEQ ID NO: 122) and one within amino acids 124-131 (SEQ ID NO: 7); or one within amino acids 82-96 (SEQ ID NO: 130) and one within amino acids 124-131 (SEQ ID NO: 7); or one within amino acids 10-24 (SEQ ID NO: 122) and one within amino acids 128-135 (SEQ ID NO: 8); or one within amino acids 82-96 (SEQ ID NO: 130) and one within amino acids 128-135 (SEQ ID NO: 8); or one within amino acids 131-140 (SEQ ID NO: 9) and one within amino acids 28-50 (SEQ ID NO: 139); or one within amino acids 131-140 (SEQ ID NO: 9) and one within amino acids 100-114 (SEQ ID NO: 132); or one within amino acids 91-105 (SEQ ID NO: 131) and one within amino acids 28-50 (SEQ ID NO: 139); or one within amino acids 28-42 (SEQ ID NO: 124) and one within amino acids 28-50 (SEQ ID NO: 139); or one within amino acids 37-51 (SEQ ID NO: 125) and one within amino acids 28-50 (SEQ ID NO: 139); or one within amino acids 28-42 (SEQ ID NO: 124) and 37-51 (SEQ ID NO: 125) and one within amino acids 28-50 (SEQ ID NO: 139); or one within amino acids 65-74 (SEQ ID NO: 4) and one within amino acids 37-51 (SEQ ID NO: 125); or one within amino acids 1-15 (SEQ ID NO: 121) and one within amino acids 128-135 (SEQ ID NO: 8); or one within amino acids 91-105 (SEQ ID NO: 131) and one within amino acids 100-114 (SEQ ID NO: 132) or one within amino acids 91-105 (SEQ ID NO: 131) and one within amino acids 109-123 (SEQ ID NO: 133); or one within amino acids 91-105 (SEQ ID NO: 131) and one within amino acids 100-114 (SEQ ID NO: 132) and 109-123 (SEQ ID NO: 133); or one within amino acids 124-131 (SEQ ID NO: 7) and one within amino acids 91-105 (SEQ ID NO: 131); or one within amino acids 100-114 (SEQ ID NO: 132) and one within amino acids 82-96 (SEQ ID NO: 130); or one within amino acids 109-123 (SEQ ID NO: 133) and one within amino acids 82-96 (SEQ ID NO: 130); or one within amino acids 100-114 (SEQ ID NO: 132) and 109-123 (SEQ ID NO: 133) and one within amino acids 82-96 (SEQ ID NO: 130); or one within amino acids 100-114 (SEQ ID NO: 132) and one within amino acids 28-50 (SEQ ID NO: 139); or one within amino acids 100-114 (SEQ ID NO: 132) and 109-123 (SEQ ID NO: 133) and one within amino acids 28-50 (SEQ ID NO: 139); or one within amino acids 109-123 (SEQ ID NO: 133) and one within amino acids 28-50 (SEQ ID NO: 139); or one within amino acids 109-123 (SEQ ID NO: 133) and one within amino acids 100-114 (SEQ ID NO: 132); or one within amino acids 100-114 (SEQ ID NO: 132) and one within amino acids 100-114 (SEQ ID NO: 132); or one within amino acids 100-114 (SEQ ID NO: 132) and 109-123 (SEQ ID NO: 133) and one within amino acids 100-114 (SEQ ID NO: 132); or one within amino acids 91-105 (SEQ ID NO: 131) and one within amino acids 1-15 (SEQ ID NO: 121); or one within amino acids 28-42 (SEQ ID NO: 124) and one within amino acids 100-114 (SEQ ID NO: 132); or one within amino acids 28-42 (SEQ ID NO: 124) and one within amino acids 109-123 (SEQ ID NO: 133); or one within amino acids 37-51 (SEQ ID NO: 125) and one within amino acids 100-114 (SEQ ID NO: 132); or one within amino acids 37-51 (SEQ ID NO: 125) and one within amino acids 109-123 (SEQ ID NO: 133); or one within amino acids 28-42 (SEQ ID NO: 124) and 37-51 (SEQ ID NO: 125) and one within amino acids 100-114 (SEQ ID NO: 132) and 109-123 (SEQ ID NO: 133); or one within amino acids 65-74 (SEQ ID NO: 4) and one within amino acids 100-114 (SEQ ID NO: 132); or one within amino acids 65-74 (SEQ ID NO: 4) and one within amino acids 109-123 (SEQ ID NO: 133); or one within amino acids 65-74 (SEQ ID NO: 4) and one within amino acids 100-114 (SEQ ID NO: 132) and 109-123 (SEQ ID NO: 133); or one within amino acids 91-105 (SEQ ID NO: 131) and one within amino acids 100-114 (SEQ ID NO: 132); or one within amino acids 91-105 (SEQ ID NO: 131) and one within amino acids 109-123 (SEQ ID NO: 133); or one within amino acids 91-105 (SEQ ID NO: 131) and one within amino acids 100-114 (SEQ ID NO: 132) and 109-123 (SEQ ID NO: 133); or one within amino acids 124-131 (SEQ ID NO: 7) and one within amino acids 100-114 (SEQ ID NO: 132); or one within amino acids 124-131 (SEQ ID NO: 7) and one within amino acids 109-123 (SEQ ID NO: 133); or one within amino acids 124-131 (SEQ ID NO: 7) and one within amino acids 100-114 (SEQ ID NO: 132) and 109-123 (SEQ ID NO: 133); or one within amino acids 81-120 (SEQ ID NO: 137) and one within amino acids 124-131 (SEQ ID NO: 7); or one within amino acids 81-120 (SEQ ID NO: 137) and one within amino acids 1-15 (SEQ ID NO: 121); or one within amino acids 81-120 (SEQ ID NO: 137) and one within amino acids 28-42 (SEQ ID NO: 124); or one within amino acids 81-120 (SEQ ID NO: 137) and one within amino acids 37-51 (SEQ ID NO: 125); or one within amino acids 81-120 (SEQ ID NO: 137) and one within amino acids 28-42 (SEQ ID NO: 124) and 37-51 (SEQ ID NO: 125); or one within amino acids 81-120 (SEQ ID NO: 137) and one within amino acids 82-96 (SEQ ID NO: 130); or one within amino acids 81-120 (SEQ ID NO: 137) and one within amino acids 91-105 (SEQ ID NO: 131); or one within amino acids 81-120 (SEQ ID NO: 137) and one within amino acids 131-140 (SEQ ID NO: 9); or one within amino acids 109-123 (SEQ ID NO: 133) and one within amino acids 131-140 (SEQ ID NO: 9); or one within amino acids 91-105 (SEQ ID NO: 131) and one within amino acids 28-50 (SEQ ID NO: 139); or one within amino acids 124-131 (SEQ ID NO: 7) and one within amino acids 1-15 (SEQ ID NO: 121); or one within amino acids 124-131 (SEQ ID NO: 7) and one within amino acids 28-50 (SEQ ID NO: 139); or one within amino acids 124-131 (SEQ ID NO: 7) and one within amino acids 82-96 (SEQ ID NO: 130); or one within amino acids 124-131 (SEQ ID NO: 7) and one within amino acids 91-105 (SEQ ID NO: 131); or one within amino acids 124-131 (SEQ ID NO: 7) and one within amino acids 100-114 (SEQ ID NO: 132).

In another embodiment, the alpha-synuclein biparatopic binding molecule of the invention in particular biparatopic antibodies or functional fragments thereof binds to two epitopes, one within amino acids 65-74 (SEQ ID NO: 4) and one within amino acids 124-131 (SEQ ID NO: 7); or one within amino acids 128-135 (SEQ ID NO: 8) and one within amino acids 124-131 (SEQ ID NO: 7) or one within amino acids 124-131 (SEQ ID NO: 7) and one within amino acids 131-140 (SEQ ID NO: 9); or one within amino acids 28-42 (SEQ ID NO: 124) and one within amino acids 28-50 (SEQ ID NO: 139); or one within amino acids 37-51 (SEQ ID NO: 125) and one within amino acids 28-50 (SEQ ID NO: 139); or one within amino acids 28-42 (SEQ ID NO: 124) and 37-51 (SEQ ID NO: 125) and one within amino acids 28-50 (SEQ ID NO: 139); or one within amino acids 28-42 (SEQ ID NO: 124) and one within amino acids 100-114 (SEQ ID NO: 132); or one within amino acids 37-51 (SEQ ID NO: 125) and one within amino acids 100-114 (SEQ ID NO: 132); or one within amino acids 28-42 (SEQ ID NO: 124) and 37-51 (SEQ ID NO: 125) and one within amino acids 100-114 (SEQ ID NO: 132); or one within amino acids 100-114 (SEQ ID NO: 132) and one within amino acids 28-50 (SEQ ID NO: 139); or one within amino acids 109-123 (SEQ ID NO: 133) and one within amino acids 28-50 (SEQ ID NO: 139); or one within amino acids 100-114 (SEQ ID NO: 132) and 109-123 (SEQ ID NO: 133) and one within amino acids 28-50 (SEQ ID NO: 139); or one within amino acids 91-105 (SEQ ID NO: 131) and one within amino acids 1-15 (SEQ ID NO: 121); or one within amino acids 124-131 (SEQ ID NO: 7) and one within amino acids 100-114 (SEQ ID NO: 132); or one within amino acids 124-131 (SEQ ID NO: 7) and one within amino acids 109-123 (SEQ ID NO: 133); or one within amino acids 124-131 (SEQ ID NO: 7) and one within amino acids 100-114 (SEQ ID NO: 132) and 109-123 (SEQ ID NO: 133); or one within amino acids 124-131 (SEQ ID NO: 7) and one within amino acids 82-96 (SEQ ID NO: 130); or one within amino acids 81-120 (SEQ ID NO: 137) and one within amino acids 28-42 (SEQ ID NO: 124); or one within amino acids 81-120 (SEQ ID NO: 137) and one within amino acids 37-51 (SEQ ID NO: 125); or one within amino acids 81-120 (SEQ ID NO: 137) and one within amino acids 28-42 (SEQ ID NO: 124) and 37-51 (SEQ ID NO: 125); or one within amino acids 28-50 (SEQ ID NO: 139) and one within amino acids 124-131 (SEQ ID NO: 7); or one within amino acids 91-105 (SEQ ID NO: 131) and one within amino acids 124-131 (SEQ ID NO: 7), or one within amino acids 100-114 (SEQ ID NO: 132) and one within amino acids 109-123 (SEQ ID NO: 133); or one within amino acids 100-114 (SEQ ID NO: 132) and one within amino acids 100-114 (SEQ ID NO: 132); or one within amino acids 100-114 (SEQ ID NO: 132) and 109-123 (SEQ ID NO: 133) and one within amino acids 100-114 (SEQ ID NO: 132); or one within amino acids 100-114 (SEQ ID NO: 132) and one within amino acids 100-114 (SEQ ID NO: 132).

In a further embodiment, the alpha-synuclein biparatopic binding molecule of the invention in particular biparatopic antibodies or functional fragments thereof binds to two epitopes, one within amino acids 124-131 (SEQ ID NO: 7) and one within amino acids 82-96 (SEQ ID NO: 130); or one within amino acids 100-114 (SEQ ID NO: 132) and one within amino acids 28-50 (SEQ ID NO: 139); or one within amino acids 109-123 (SEQ ID NO: 133) and one within amino acids 28-50 (SEQ ID NO: 139); or one within amino acids 100-114 (SEQ ID NO: 132) and 109-123 (SEQ ID NO: 133) and one within amino acids 28-50 (SEQ ID NO: 139); or one within amino acids 100-114 (SEQ ID NO: 132) and one within amino acids 109-123 (SEQ ID NO: 133); or one within amino acids 100-114 (SEQ ID NO: 132) and one within amino acids 100-114 (SEQ ID NO: 132) and 109-123 (SEQ ID NO: 133) or one within amino acids 100-114 (SEQ ID NO: 132) and one within amino acids 100-114 (SEQ ID NO: 132).

In a further embodiment, the alpha-synuclein biparatopic binding molecule of the invention in particular biparatopic antibodies or functional fragments thereof binds to two epitopes, one within amino acids 124-131 (SEQ ID NO: 7) and one within amino acids 82-96 (SEQ ID NO: 130); or one within amino acids 100-114 (SEQ ID NO: 132) and one within amino acids 100-114 (SEQ ID NO: 132). In the second case, although the epitopes are within the same region they are distinct.

The epitopes may be further defined according to critical amino acid residues within the epitopes that are bound by the antibodies or functional fragments thereof. These residues can be defined for example by alanine scanning. Results of such experiments are described in the examples below, see Table 4 and are applicable to all relevant embodiments. Thus, in some embodiments, the alpha-synuclein biparatopic antibody or functional fragment thereof, or a mixture comprising at least two monospecific antibodies or functional fragments thereof, of the invention comprises a first binding site which binds a first epitope and a second distinct binding site which binds to a second distinct epitope, wherein:
a. the first epitope is situated within amino acid residues 82-96 (SEQ ID NO: 130) of human alpha-synuclein of SEQ ID NO: 1 and critical amino acid residues for binding comprise, or consist of, amino acid residues 92-94 and 96 and the second epitope is situated within amino acid residues 124-131 (SEQ ID NO: 7) of human alpha-synuclein of SEQ ID NO: 1 and critical amino acid residues for binding comprise, or consist of, amino acid residues 126-127; or
b. the first and second epitope are situated within amino acid residues 100-114 (SEQ ID NO: 132) of human alpha-synuclein of SEQ ID NO: 1 and critical amino acid residues for binding within the first epitope comprise, or consist of, amino acid residues 100-105. The second epitope is distinct and thus the critical residues do not consist of amino acid residues 100-105.

Biparatopic antibodies or functional fragments thereof binding one of the epitopes of any of the biparatopic antibodies provided herein are also part of the invention. By this is meant that biparatopic antibodies or functional fragments thereof are encompassed by the invention that bind to the same epitopes as those bound by the biparatopic antibodies or functional fragments thereof specifically disclosed herein. This may be measured for example by the ability of the antibodies or functional fragments to compete for binding to the epitope. Suitable competition assays are described herein. In some embodiments, a biparatopic antibody or a functional fragment thereof is provided, wherein the biparatopic antibody or a functional fragment thereof comprises one binding site which binds to or within the epitope comprising the sequence of SEQ ID NO: 2. In some embodiments, a biparatopic antibody or a functional fragment thereof is provided, wherein the biparatopic antibody or a functional fragment thereof comprises one binding site which binds to or within an epitope comprising the sequence of SEQ ID NO: 3. In some embodiments, a biparatopic antibody or a functional fragment thereof is provided, wherein the biparatopic antibody or a functional fragment thereof comprises one binding site which binds to or within an epitope comprising the sequence of SEQ ID NO: 4. In some embodiments, a biparatopic antibody or a functional fragment thereof is provided, wherein the biparatopic antibody or a functional fragment thereof comprises one binding site which binds to or within an epitope comprising the sequence of SEQ ID NO: 5. In some embodiments, a biparatopic antibody or a functional fragment thereof is provided, wherein the biparatopic antibody or a functional fragment thereof comprises one binding site which binds to or within an epitope comprising the sequence comprising amino acids 93-95 of SEQ ID NO: 1. In some embodiments, a biparatopic antibody or a functional fragment thereof is provided, wherein the biparatopic antibody or a functional fragment thereof comprises one binding site which binds to or within an epitope comprising the sequence of SEQ ID NO: 7. In some embodiments, a biparatopic antibody or a functional fragment thereof is provided, wherein the biparatopic antibody or a functional fragment thereof comprises one binding site which binds to or within an epitope comprising the sequence of SEQ ID NO: 8. In some embodiments, a biparatopic antibody or a functional fragment thereof is provided, wherein the biparatopic antibody or a functional fragment thereof comprises one binding site which binds to or within an epitope comprising the sequence of SEQ ID NO: 9. In some embodiments, a biparatopic antibody or a functional fragment thereof is provided, wherein the biparatopic antibody or a functional fragment thereof comprises one binding site which binds to or within an epitope comprising the sequence of SEQ ID NO: 121. In some embodiments, a biparatopic antibody or a functional fragment thereof is provided, wherein the biparatopic antibody or a functional fragment thereof comprises one binding site which binds to or within an epitope comprising the sequence of SEQ ID NO: 136. In some embodiments, a biparatopic antibody or a functional fragment thereof is provided, wherein the biparatopic antibody or a functional fragment thereof comprises one binding site which binds to or within an epitope comprising the sequence of SEQ ID NO: 130. In some embodiments, a biparatopic antibody or a functional fragment thereof is provided, wherein the biparatopic antibody or a functional fragment thereof comprises one binding site which binds to or within an epitope comprising the sequence of SEQ ID NO: 131. In some embodiments, a biparatopic antibody or a functional fragment thereof is provided, wherein the biparatopic antibody or a functional fragment thereof comprises one binding site which binds to or within an epitope comprising the sequence of SEQ ID NO: 134. In some embodiments, a biparatopic antibody or a functional fragment thereof is provided, wherein the biparatopic antibody or a functional fragment thereof comprises one binding site which binds to or within an epitope comprising the sequence of SEQ ID NO: 135. In some embodiments, a biparatopic antibody or a functional fragment thereof is provided, wherein the biparatopic antibody or a functional fragment thereof comprises one binding site which binds to or within an epitope comprising the sequence of SEQ ID NO: 122. In some embodiments, a biparatopic antibody or a functional fragment thereof is provided, wherein the biparatopic antibody or a functional fragment thereof comprises one binding site which binds to or within an epitope comprising the sequence of SEQ ID NO: 124. In some embodiments, a biparatopic antibody or a functional fragment thereof is provided, wherein the biparatopic antibody or a functional fragment thereof comprises one binding site which binds to or within an epitope comprising the sequence of SEQ ID NO: 125. In some embodiments, a biparatopic antibody or a functional fragment thereof is provided, wherein the biparatopic antibody or a functional fragment thereof comprises one binding site which binds to or within an epitope comprising the sequence of SEQ ID NO: 131. In some embodiments, a biparatopic antibody or a functional fragment thereof is provided, wherein the biparatopic antibody or a functional fragment thereof comprises one binding site which binds to or within an epitope comprising the sequence of SEQ ID NO: 132. In some embodiments, a biparatopic antibody or a functional fragment thereof is provided, wherein the biparatopic antibody or a functional fragment thereof comprises one binding site which binds to or within an epitope comprising the sequence of SEQ ID NO: 133. In some embodiments, a biparatopic antibody or a functional fragment thereof is provided, wherein the biparatopic antibody or a functional fragment thereof comprises one binding site which binds to or within an epitope comprising the sequence of SEQ ID NO: 137. In some embodiments, a biparatopic antibody or a functional fragment thereof is provided, wherein the biparatopic antibody or a functional fragment thereof comprises one binding site which binds to or within an epitope comprising the sequence of SEQ ID NO: 138. In some embodiments, a biparatopic antibody or a functional fragment thereof is provided, wherein the biparatopic antibody or a functional fragment thereof comprises one binding site which binds to or within an epitope comprising the sequence of SEQ ID NO: 139. In some embodiments, a biparatopic antibody or a functional fragment thereof is provided, wherein the biparatopic antibody or a functional fragment thereof comprises one binding site which binds to or within an epitope comprising the sequence of SEQ ID NO: 146. In some embodiments, a biparatopic antibody or a functional fragment thereof is provided, wherein the biparatopic antibody or a functional fragment thereof comprises one binding site which binds to or within an epitope comprising the sequence of SEQ ID NO: 147. In some embodiments, a biparatopic antibody or a functional fragment thereof is provided wherein the bipratopic antibody or a functional fragment thereof comprises one binding site which binds to or within a non-linear epitope within amino acids residues of human alpha-synuclein of SEQ ID NO: 1.

In some embodiments, a biparatopic antibody or a functional fragment thereof binds within epitope as at least one antibody, more particularly at least two antibodies selected from ACI-7067-1101C8-Ab2, ACI-7067-1102G3-Ab1, ACI-7067-1106A8-Ab2, ACI-7067-1107G5-Ab2, ACI-7067-1108H1-Ab1, ACI-7067-1111B12-Ab2, ACI-7067-1112H8-Ab2, ACI-7067-1108B11-Ab2, ACI-7067-1113D10-Ab1, ACI-7067-1116F2-Ab1, ACI-7067-1206E5-Ab1, ACI-7079-2501B11-Ab3, ACI-7079-2501D10-Ab1, ACI-7079-2501G2-Ab2, ACI-7079-2503C6-Ab1, ACI-7079-2504A6-Ab1, ACI-7079-2506E2-Ab2, ACI-7079-2506F3-Ab1, ACI-7079-2507B3-Ab1, ACI-7079-2511B3-Ab3, ACI-7079-2601B6-Ab1, ACI-7079-2602G4-Ab4, ACI-7079-2603C1-Ab3, ACI-7079-2603F3-Ab1, ACI-7079-2605B3-Ab2, ACI-7079-2606A6-Ab2, ACI-7079-2509E5-Ab2, ACI-7087-4119E10-Ab2, ACI-7087-4125E6-Ab1, ACI-7088-4301D5-Ab2, ACI-7088-4301E12-Ab2, ACI-7088-4301H3-Ab2, ACI-7088-4303A1-Ab1, ACI-7088-4303A3-Ab1, ACI-7088-4303B6-Ab2, ACI-7088-4303H6-Ab1, ACI-7088-4305H7-Ab1, ACI-7088-4317A4-Ab1, ACI-7089-4409F1-Ab1, ACI-7089-4415G5-Ab1, ACI-7089-4417G6-Ab1, ACI-7089-4418C5-Ab1, ACI-7089-4418F6-Ab1, ACI-8033-5A12-Ab1, ACI-8033-25A3-Ab1, ACI-8033-1G10-Ab1, ACI-8033-19A2-Ab1, ACI-8033-8C10-Ab1, ACI-8033-7A2-Ab1, ACI-8033-1A12-Ab1, ACI-8033-4F3-Ab1, ACI-8033-17F5-Ab1, ACI-8033-18C11-Ab1, ACI-8033-18D12-Ab1, ACI-8033-1F8-Ab1, ACI-8033-22E5-Ab1, ACI-8033-27D8-Ab1, ACI-8033-21C8-Ab1, ACI-7079-3101E3-Ab1, ACI-7079-3103D9-Ab1, ACI-7079-3103G12-Ab2, ACI-7079-3104F12-Ab2, ACI-7079-3106C5-Ab1, ACI-7079-3106F2-Ab1, ACI-7079-3112H1-Ab1, ACI-7079-3107E6-Ab1, ACI-7079-3108C10-Ab2, ACI-8030-6106F5-Ab1, ACI-8031-6207G10-Ab1, ACI-8032-6301A10-Ab2, ACI-8032-6301C8-Ab2, ACI-8032-6301G2-Ab2, ACI-8032-6304F3-Ab1, ACI-8032-6307F1-Ab2, ACI-8032-6313G2-Ab1, ACI-8032-6314A3-Ab3, ACI-8033-6401F2-Ab1, ACI-8033-6402E2-Ab2, ACI-8033-6402E10-Ab1, ACI-8033-6403A4-Ab1, ACI-8033-6403E11-Ab2 and ACI-7067-4813-R4A-G7-rec1.

In some embodiments, a biparatopic antibody or a functional fragment thereof competes with binding to alpha-synuclein with at least one antibody selected from ACI-7067-1101C8-Ab2, ACI-7067-1102G3-Ab1, ACI-7067-1106A8-Ab2, ACI-7067-1107G5-Ab2, ACI-7067-1108H1-Ab1, ACI-7067-1111B12-Ab2, ACI-7067-1112H8-Ab2, ACI-7067-1108B11-Ab2, ACI-7067-1113D10-Ab1, ACI-7067-1116F2-Ab1, ACI-7067-1206E5-Ab1, ACI-7079-2501B11-Ab3, ACI-7079-2501D10-Ab1, ACI-7079-2501G2-Ab2, ACI-7079-2503C6-Ab1, ACI-7079-2504A6-Ab1, ACI-7079-2506E2-Ab2, ACI-7079-2506F3-Ab1, ACI-7079-2507B3-Ab1, ACI-7079-2511B3-Ab3, ACI-7079-2601B6-Ab1, ACI-7079-2602G4-Ab4, ACI-7079-2603C1-Ab3, ACI-7079-2603F3-Ab1, ACI-7079-2605B3-Ab2, ACI-7079-2606A6-Ab2, ACI-7079-2509E5-Ab2, ACI-7087-4119E10-Ab2, ACI-7087-4125E6-Ab1, ACI-7088-4301D5-Ab2, ACI-7088-4301E12-Ab2, ACI-7088-4301H3-Ab2, ACI-7088-4303A1-Ab1, ACI-7088-4303A3-Ab1, ACI-7088-4303B6-Ab2, ACI-7088-4303H6-Ab1, ACI-7088-4305H7-Ab1, ACI-7088-4317A4-Ab1, ACI-7089-4409F1-Ab1, ACI-7089-4415G5-Ab1, ACI-7089-4417G6-Ab1, ACI-7089-4418C5-Ab1, ACI-7089-4418F6-Ab1, ACI-8033-5A12-Ab1, ACI-8033-25A3-Ab1, ACI-8033-1G10-Ab1, ACI-8033-19A2-Ab1, ACI-8033-8C10-Ab1, ACI-8033-7A2-Ab1, ACI-8033-1A12-Ab1, ACI-8033-4F3-Ab1, ACI-8033-17F5-Ab1, ACI-8033-18C11-Ab1, ACI-8033-18D12-Ab1, ACI-8033-1F8-Ab1, ACI-8033-22E5-Ab1, ACI-8033-27D8-Ab1, ACI-8033-21C8-Ab1, ACI-7079-3101E3-Ab1, ACI-7079-3103D9-Ab1, ACI-7079-3103G12-Ab2, ACI-7079-3104F12-Ab2, ACI-7079-3106C5-Ab1, ACI-7079-3106F2-Ab1, ACI-7079-3112H1-Ab1, ACI-7079-3107E6-Ab1, ACI-7079-3108C10-Ab2, ACI-8030-6106F5-Ab1, ACI-8031-6207G10-Ab1, ACI-8032-6301A10-Ab2, ACI-8032-6301C8-Ab2, ACI-8032-6301G2-Ab2, ACI-8032-6304F3-Ab1, ACI-8032-6307F1-Ab2, ACI-8032-6313G2-Ab1, ACI-8032-6314A3-Ab3, ACI-8033-6401F2-Ab1, ACI-8033-6402E2-Ab2, ACI-8033-6402E10-Ab1, ACI-8033-6403A4-Ab1, ACI-8033-6403E11-Ab2 and ACI-7067-4813-R4A-G7-rec1.

In some embodiments of the invention, a mixture comprises monospecific monoclonal antibodies or functional fragments thereof, mixtures of biparatopic antibodies or functional fragments thereof and at least one monospecific monoclonal antibody or a functional fragment thereof. In some embodiments of the invention, a mixture comprises at least two alpha-synuclein monospecific binding molecules of the invention, or at least three alpha-synuclein monospecific binding molecules of the invention, or at least four alpha-synuclein monospecific binding molecules of the invention, or at least five alpha-synuclein monospecific binding molecules of the invention.

In some embodiments of the invention, the biparatopic binding molecule, particularly the biparatopic antibody or a functional fragment thereof comprises at least one binding site comprising the variable regions VH and/or VL of the amino acid sequences, respectively, set forth in SEQ ID NO: 10 and SEQ ID NO: 14; SEQ ID NO: 20 and SEQ ID NO: 24; SEQ ID NO: 30 and SEQ ID NO: 34; SEQ ID NO: 40 and SEQ ID NO: 44; SEQ ID NO: 50 and SEQ ID NO: 54; SEQ ID NO: 60 and SEQ ID NO: 64; SEQ ID NO: 70 and SEQ ID NO: 74; SEQ ID NO: 30 and SEQ ID NO: 84; SEQ ID NO: 90 and SEQ ID NO: 94; SEQ ID NO: 100 and SEQ ID NO: 104; SEQ ID NO: 110 and SEQ ID NO: 114; SEQ ID NO: 280 and SEQ ID NO: 284; SEQ ID NO: 290 and SEQ ID NO: 194; SEQ ID NO: 140 and SEQ ID NO: 144; SEQ ID NO: 150 and SEQ ID NO: 154; SEQ ID NO: 160 and SEQ ID NO: 164; SEQ ID NO: 170 and SEQ ID NO: 174; SEQ ID NO: 180 and SEQ ID NO: 184; SEQ ID NO: 190 and SEQ ID NO: 194; SEQ ID NO: 200 and SEQ ID NO: 204; SEQ ID NO: 210 and SEQ ID NO: 214; SEQ ID NO: 220 and SEQ ID NO: 224; SEQ ID NO: 230 and SEQ ID NO: 234; SEQ ID NO: 240 and SEQ ID NO: 244; SEQ ID NO: 250 and SEQ ID NO: 254; SEQ ID NO: 260 and SEQ ID NO: 264; SEQ ID NO: 270 and SEQ ID NO: 274; SEQ ID NO: 300 and SEQ ID NO: 304; SEQ ID NO: 310 and SEQ ID NO: 314; SEQ ID NO: 320 and SEQ ID NO: 324; SEQ ID NO: 330 and SEQ ID NO: 334; SEQ ID NO: 340 and SEQ ID NO: 344; SEQ ID NO: 350 and SEQ ID NO: 354; SEQ ID NO: 360 and SEQ ID NO: 364; SEQ ID NO: 370 and SEQ ID NO: 374; SEQ ID NO: 380 and SEQ ID NO: 384; SEQ ID NO: 390 and SEQ ID NO: 394; SEQ ID NO: 400 and SEQ ID NO: 404; SEQ ID NO: 410 and SEQ ID NO: 414; SEQ ID NO: 420 and SEQ ID NO: 424; SEQ ID NO: 430 and SEQ ID NO: 434; SEQ ID NO: 440 and SEQ ID NO: 414; SEQ ID NO: 450 and SEQ ID NO: 424; SEQ ID NO: 460 and SEQ ID NO: 464; SEQ ID NO: 470 and SEQ ID NO: 474; SEQ ID NO: 480 and SEQ ID NO: 484; SEQ ID NO: 490 and SEQ ID NO: 494; SEQ ID NO: 500 and SEQ ID NO: 504; SEQ ID NO: 510 and SEQ ID NO: 514; SEQ ID NO: 520 and SEQ ID NO: 524; SEQ ID NO: 530 and SEQ ID NO: 534; SEQ ID NO: 540 and SEQ ID NO: 544; SEQ ID NO: 550 and SEQ ID NO: 554; SEQ ID NO: 560 and SEQ ID NO: 564; SEQ ID NO: 570 and SEQ ID NO: 574; SEQ ID NO: 580 and SEQ ID NO: 584; SEQ ID NO: 590 and SEQ ID NO: 474; SEQ ID NO: 600 and SEQ ID NO: 554; SEQ ID NO: 610 and SEQ ID NO: 614; SEQ ID NO: 620 and SEQ ID NO: 624; SEQ ID NO: 630 and SEQ ID NO: 634; SEQ ID NO: 640 and SEQ ID NO: 644; SEQ ID NO: 650 and SEQ ID NO: 654; SEQ ID NO: 660 and SEQ ID NO: 664; SEQ ID NO: 670 and SEQ ID NO: 674; SEQ ID NO: 680 and SEQ ID NO: 684; SEQ ID NO: 690 and SEQ ID NO: 694; SEQ ID NO: 700 and SEQ ID NO: 704; SEQ ID NO: 710 and SEQ ID NO: 714; SEQ ID NO: 720 and SEQ ID NO: 724; SEQ ID NO: 730 and SEQ ID NO: 734; SEQ ID NO: 740 and SEQ ID NO: 744; SEQ ID NO: 750 and SEQ ID NO: 754; SEQ ID NO: 760 and SEQ ID NO: 764; SEQ ID NO: 770 and SEQ ID NO: 774; SEQ ID NO: 750 and SEQ ID NO: 784; SEQ ID NO: 790 and SEQ ID NO: 794; SEQ ID NO: 800 and SEQ ID NO: 804; SEQ ID NO: 810 and SEQ ID NO: 814; SEQ ID NO: 820 and SEQ ID NO: 824; SEQ ID NO: 830 and SEQ ID NO: 834; SEQ ID NO: 840 and SEQ ID NO: 844.

In particular, in some embodiments of the invention, the biparatopic antibody, or a functional fragment thereof comprises at least one binding site comprising the variable regions VH and/or VL of the amino acid sequences, respectively, set forth in SEQ ID NO: 10 and SEQ ID NO: 14; SEQ ID NO: 30 and SEQ ID NO: 84; SEQ ID NO: 50 and SEQ ID NO: 54; SEQ ID NO: 90 and SEQ ID NO: 94; SEQ ID NO: 150 and SEQ ID NO: 154; SEQ ID NO: 180 and SEQ ID NO: 184; SEQ ID NO: 690 and SEQ ID NO: 694; SEQ ID NO: 670 and SEQ ID NO: 674; SEQ ID NO: 320 and SEQ ID NO: 324; SEQ ID NO: 360 and SEQ ID NO: 364; SEQ ID NO: 400 and SEQ ID NO: 404; SEQ ID NO: 530 and SEQ ID NO: 534; SEQ ID NO: 460 and SEQ ID NO: 464; SEQ ID NO: 590 and SEQ ID NO: 474; SEQ ID NO: 570 and SEQ ID NO: 574; SEQ ID NO: 340 and SEQ ID NO: 344; SEQ ID NO: 510 and SEQ ID NO: 514; SEQ ID NO: 330 and SEQ ID NO: 334; SEQ ID NO: 610 and SEQ ID NO: 614.

In some embodiment of the invention, a biparatopic binding molecule, particularly a biparatopic antibody, or a functional fragment thereof comprises a first binding site comprising a pair of variable regions VH and VL and a second binding site comprising a distinct pair of variable regions VH and VL selected from the group consisting of the pair of variable regions VH and VL of the amino acid sequences respectively, set forth in SEQ ID NO: 10 and SEQ ID NO: 14; SEQ ID NO: 20 and SEQ ID NO: 24; SEQ ID NO: 30 and SEQ ID NO: 34; SEQ ID NO: 40 and SEQ ID NO: 44; SEQ ID NO: 50 and SEQ ID NO: 54; SEQ ID NO: 60 and SEQ ID NO: 64; SEQ ID NO: 70 and SEQ ID NO: 74; SEQ ID NO: 30 and SEQ ID NO: 84; SEQ ID NO: 90 and SEQ ID NO: 94; SEQ ID NO: 100 and SEQ ID NO: 104; SEQ ID NO: 110 and SEQ ID NO: 114; SEQ ID NO: 280 and SEQ ID NO: 284; SEQ ID NO: 290 and SEQ ID NO: 194; SEQ ID NO: 140 and SEQ ID NO: 144; SEQ ID NO: 150 and SEQ ID NO: 154; SEQ ID NO: 160 and SEQ ID NO: 164; SEQ ID NO: 170 and SEQ ID NO: 174; SEQ ID NO: 180 and SEQ ID NO: 184; SEQ ID NO: 190 and SEQ ID NO: 194; SEQ ID NO: 200 and SEQ ID NO: 204; SEQ ID NO: 210 and SEQ ID NO: 214; SEQ ID NO: 220 and SEQ ID NO: 224; SEQ ID NO: 230 and SEQ ID NO: 234; SEQ ID NO: 240 and SEQ ID NO: 244; SEQ ID NO: 250 and SEQ ID NO: 254; SEQ ID NO: 260 and SEQ ID NO: 264; SEQ ID NO: 270 and SEQ ID NO: 274; SEQ ID NO: 300 and SEQ ID NO: 304; SEQ ID NO: 310 and SEQ ID NO: 314; SEQ ID NO: 320 and SEQ ID NO: 324; SEQ ID NO: 330 and SEQ ID NO: 334; SEQ ID NO: 340 and SEQ ID NO: 344; SEQ ID NO: 350 and SEQ ID NO: 354; SEQ ID NO: 360 and SEQ ID NO: 364; SEQ ID NO: 370 and SEQ ID NO: 374; SEQ ID NO: 380 and SEQ ID NO: 384; SEQ ID NO: 390 and SEQ ID NO: 394; SEQ ID NO: 400 and SEQ ID NO: 404; SEQ ID NO: 410 and SEQ ID NO: 414; SEQ ID NO: 420 and SEQ ID NO: 424; SEQ ID NO: 430 and SEQ ID NO: 434; SEQ ID NO: 440 and SEQ ID NO: 414; SEQ ID NO: 450 and SEQ ID NO: 424; SEQ ID NO: 460 and SEQ ID NO: 464; SEQ ID NO: 470 and SEQ ID NO: 474; SEQ ID NO: 480 and SEQ ID NO: 484; SEQ ID NO: 490 and SEQ ID NO: 494; SEQ ID NO: 500 and SEQ ID NO: 504; SEQ ID NO: 510 and SEQ ID NO: 514; SEQ ID NO: 520 and SEQ ID NO: 524; SEQ ID NO: 530 and SEQ ID NO: 534; SEQ ID NO: 540 and SEQ ID NO: 544; SEQ ID NO: 550 and SEQ ID NO: 554; SEQ ID NO: 560 and SEQ ID NO: 564; SEQ ID NO: 570 and SEQ ID NO: 574; SEQ ID NO: 580 and SEQ ID NO: 584; SEQ ID NO: 590 and SEQ ID NO: 474; SEQ ID NO: 600 and SEQ ID NO: 554; SEQ ID NO: 610 and SEQ ID NO: 614; SEQ ID NO: 620 and SEQ ID NO: 624; SEQ ID NO: 630 and SEQ ID NO: 634; SEQ ID NO: 640 and SEQ ID NO: 644; SEQ ID NO: 650 and SEQ ID NO: 654; SEQ ID NO: 660 and SEQ ID NO: 664; SEQ ID NO: 670 and SEQ ID NO: 674; SEQ ID NO: 680 and SEQ ID NO: 684; SEQ ID NO: 690 and SEQ ID NO: 694; SEQ ID NO: 700 and SEQ ID NO: 704; SEQ ID NO: 710 and SEQ ID NO: 714; SEQ ID NO: 720 and SEQ ID NO: 724; SEQ ID NO: 730 and SEQ ID NO: 734; SEQ ID NO: 740 and SEQ ID NO: 744; SEQ ID NO: 750 and SEQ ID NO: 754; SEQ ID NO: 760 and SEQ ID NO: 764; SEQ ID NO: 770 and SEQ ID NO: 774; SEQ ID NO: 750 and SEQ ID NO: 784; SEQ ID NO: 790 and SEQ ID NO: 794; SEQ ID NO: 800 and SEQ ID NO: 804; SEQ ID NO: 810 and SEQ ID NO: 814; SEQ ID NO: 820 and SEQ ID NO: 824; SEQ ID NO: 830 and SEQ ID NO: 834; SEQ ID NO: 840 and SEQ ID NO: 844.

In some embodiment of the invention an alpha-synuclein biparatopic antibody or functional fragment thereof comprises at least one pair, in particular at least two distinct pairs, of variable regions Heavy Chain Variable Region (VH) and Light Chain Variable Region (VL), wherein:
a) the VH has at least 96%, 97%, 98%, 99% or 100% sequence identity to the amino acid sequence of SEQ ID NO: 10; and the VL has at least 99% or 100% sequence identity to the amino acid sequence of SEQ ID NO: 14; or
b) the VH has at least 96%, 97%, 98%, 99% or 100% sequence identity to the amino acid sequence of SEQ ID NO: 20; and the VL has at least 93%, 94%, 95%, 96%, 97%, 98%, 99% or 100% sequence identity to the amino acid sequence of SEQ ID NO: 24; or
c) the VH having at least 94%, 95%, 96%, 97%, 98%, 99% or 100% sequence identity to the amino acid sequence of SEQ ID NO: 30; and the VL has at least 98%, 99% or 100% sequence identity to the amino acid sequence of SEQ ID NO: 34; or
d) the VH has at least 94%, 95%, 96%, 97%, 98%, 99% or 100% sequence identity to the amino acid sequence of SEQ ID NO: 40; and the VL has at least 94%, 95%, 96%, 97%, 98%, 99% or 100% sequence identity to the amino acid sequence of SEQ ID NO: 44; or
e) the VH having at least 90%, 91%, 92%, 93%, 94%, 95%, 96%, 97%, 98%, 99% or 100% sequence identity to the amino acid sequence of SEQ ID NO: 50; and the VL has at least 93%, 94%, 95%, 96%, 97%, 98%, 99% or 100% sequence identity to the amino acid sequence of SEQ ID NO: 54; or
f) the VH has at least 89%, 90%, 91%, 92%, 93%, 94%, 95%, 96%, 97%, 98%, 99% or 100% sequence identity to the amino acid sequence of SEQ ID NO: 60; and the VL has at least 96%, 97%, 98%, 99% or 100% sequence identity to the amino acid sequence of SEQ ID NO: 64; or
g) the VH has at least 91%, 92%, 93%, 94%, 95%, 96%, 97%, 98%, 99% or 100% sequence identity to the amino acid sequence of SEQ ID NO: 70; and the VL has at least 90%, 91%, 92%, 93%, 94%, 95%, 96%, 97%, 98%, 99% or 100% sequence identity to the amino acid sequence of SEQ ID NO: 74; or
h) the VH has at least 94%, 95%, 96%, 97%, 98%, 99% or 100% sequence identity to the amino acid sequence of SEQ ID NO: 30; and the VL has at least 94%, 95%, 96%, 97%, 98%, 99% or 100% sequence identity to the amino acid sequence of SEQ ID NO: 84; or
i) the VH has at least 94%, 95%, 96%, 97%, 98%, 99% or 100% sequence identity to the amino acid sequence of SEQ ID NO: 90; and the VL has at least 99% or 100% sequence identity to the amino acid sequence of SEQ ID NO: 94; or
j) the VH has at least 87%, 88%, 89%, 90%, 91%, 92%, 93%, 94%, 95%, 96%, 97%, 98%, 99% or 100% sequence identity to the amino acid sequence of SEQ ID NO: 100; and the VL has at least 98%, 99% or 100% sequence identity to the amino acid sequence of SEQ ID NO: 104; or
k) the VH has at least 97%, 98%, 99% or 100% sequence identity to the amino acid sequence of SEQ ID NO: 110; and the VL comprises the sequence of SEQ ID NO: 114; or
l) the VH has at least 91%, 92%, 93%, 94%, 95%, 96%, 97%, 98%, 99% or 100% sequence identity to the amino acid sequence of SEQ ID NO: 280; and the VL comprises the sequence of SEQ ID NO: 284; or
m) the VH has at least 91%, 92%, 93%, 94%, 95%, 96%, 97%, 98%, 99% or 100% sequence identity to the amino acid sequence of SEQ ID NO: 290; and the VL has at least 95%, 96%, 97%, 98%, 99% or 100% sequence identity to the amino acid sequence of SEQ ID NO: 194; or
n) the VH has at least 94%, 95%, 96%, 97%, 98%, 99% or 100% sequence identity to the amino acid sequence of SEQ ID NO: 140; and the VL has at least 97%, 98%, 99% or 100% sequence identity to the amino acid sequence of SEQ ID NO: 144; or
o) the VH has at least 90%, 91%, 92%, 93%, 94%, 95%, 96%, 97%, 98%, 99% or 100% sequence identity to the amino acid sequence of SEQ ID NO: 150; and the VL has at least 98%, 99% or 100% sequence identity to the amino acid sequence of SEQ ID NO: 154; or
p) the VH has at least 97%, 98%, 99% or 100% sequence identity to the amino acid sequence of SEQ ID NO: 160; and the VL comprises the sequence of SEQ ID NO: 164; or
q) the VH has at least 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 170; and the VL has at least 93%, 94%, 95%, 96%, 97%, 98%, 99% or 100% sequence identity to the amino acid sequence of SEQ ID NO: 174; or
r) the VH has at least 93%, 94%, 95%, 96%, 97%, 98%, 99% or 100% sequence identity to the amino acid sequence of SEQ ID NO: 180; and the VL comprises the sequence of SEQ ID NO: 184; or
s) the VH has at least 88%, 89%, 90%, 91%, 92%, 93%, 94%, 95%, 96%, 97%, 98%, 99% or 100% sequence identity to the amino acid sequence of SEQ ID NO: 190; and the VL has at least 95%, 96%, 97%, 98%, 99% or 100% sequence identity to the amino acid sequence of SEQ ID NO: 194; or
t) the VH has at least 93%, 94%, 95%, 96%, 97%, 98%, 99% or 100% sequence identity to the amino acid sequence of SEQ ID NO: 200; and the VL has at least 97%, 98%, 99% or 100% sequence identity to the amino acid sequence of SEQ ID NO: 204; or
u) the VH has at least 91%, 92%, 93%, 94%, 95%, 96%, 97%, 98%, 99% or 100% sequence identity to the amino acid sequence of SEQ ID NO: 210; and the VL has at least 97%, 98%, 99% or 100% sequence identity to the amino acid sequence of SEQ ID NO: 214; or
v) the VH has at least 97%, 98%, 99% or 100% sequence identity to the amino acid sequence of SEQ ID NO: 220; and the VL having at least 92%, 93%, 94%, 95%, 96%, 97%, 98%, 99% or 100% sequence identity to the amino acid sequence of SEQ ID NO: 224; or
w) the VH has at least 90%, 91%, 92%, 93%, 94%, 95%, 96%, 97%, 98%, 99% or 100% sequence identity to the amino acid sequence of SEQ ID NO: 230; and the VL has at least 97%, 98%, 99% or 100% sequence identity to the amino acid sequence of SEQ ID NO: 234; or
x) the VH has at least 96%, 97%, 98%, 99% or 100% sequence identity to the amino acid sequence of SEQ ID NO: 240; and the VL has at least 94%, 95%, 96%, 97%, 98%, 99% or 100% sequence identity to the amino acid sequence of SEQ ID NO: 244; or
y) the VH has at least 90%, 91%, 92%, 93%, 94%, 95%, 96%, 97%, 98%, 99% or 100% sequence identity to the amino acid sequence of SEQ ID NO: 250; and the VL has at least 94%, 95%, 96%, 97%, 98%, 99% or 100% sequence identity to the amino acid sequence of SEQ ID NO: 254; or
z) the VH has at least 92%, 93%, 94%, 95%, 96%, 97%, 98%, 99% or 100% sequence identity to the amino acid sequence of SEQ ID NO: 260; and the VL has at least 96%, 97%, 98%, 99% or 100% sequence identity to the amino acid sequence of SEQ ID NO: 264; or
aa) the VH has at least 85%, 86%, 87%, 88%, 89%, 90%, 91%, 92%, 93%, 94%, 95%, 96%, 97%, 98%, 99% or 100% sequence identity to the amino acid sequence of SEQ ID NO: 270; and the VL has at least 91%, 92%, 93%, 94%, 95%, 96%, 97%, 98%, 99% or 100% sequence identity to the amino acid sequence of SEQ ID NO: 274; or
bb) the VH has at least 97%, 98%, 99% or 100% sequence identity to the amino acid sequence of SEQ ID NO: 300; and the VL has at least 97%, 98%, 99% or 100% sequence identity to the amino acid sequence of SEQ ID NO: 304; or
cc) the VH has at least 94%, 95%, 96%, 97%, 98%, 99% or 100% sequence identity to the amino acid sequence of SEQ ID NO: 310; and the VL has at least 96%, 97%, 98%, 99% or 100% sequence identity to the amino acid sequence of SEQ ID NO: 314; or
dd) the VH has at least 94%, 95%, 96%, 97%, 98%, 99% or 100% sequence identity to the amino acid sequence of SEQ ID NO: 320; and the VL has at least 99% or 100% sequence identity to the amino acid sequence of SEQ ID NO: 324; or
ee) the VH has at least 96%, 97%, 98%, 99% or 100% sequence identity to the amino acid sequence of SEQ ID NO: 330; and the VL has at least 97%, 98%, 99% or 100% sequence identity to the amino acid sequence of SEQ ID NO: 334; or
ff) the VH has at least 93%, 94%, 95%, 96%, 97%, 98%, 99% or 100% sequence identity to the amino acid sequence of SEQ ID NO: 340; and the VL has at least 98%, 99% or 100% sequence identity to the amino acid sequence of SEQ ID NO: 344; or
gg) the VH has at least 92%, 93%, 94%, 95%, 96%, 97%, 98%, 99% or 100% sequence identity to the amino acid sequence of SEQ ID NO: 350; and the VL has at least 95%, 96%, 97%, 98%, 99% or 100% sequence identity to the amino acid sequence of SEQ ID NO: 354; or
hh) the VH has at least 93%, 94%, 95%, 96%, 97%, 98%, 99% or 100% sequence identity to the amino acid sequence of SEQ ID NO: 360; and the VL has at least 99% or 100% sequence identity to the amino acid sequence of SEQ ID NO: 364; or
ii) the VH has at least 90%, 91%, 92%, 93%, 94%, 95%, 96%, 97%, 98%, 99% or 100% sequence identity to the amino acid sequence of SEQ ID NO: 370; and the VL has at least 98%, 99% or 100% sequence identity to the amino acid sequence of SEQ ID NO: 374; or
jj) the VH has at least 92%, 93%, 94%, 95%, 96%, 97%, 98%, 99% or 100% sequence identity to the amino acid sequence of SEQ ID NO: 380; and the VL has at least 95%, 96%, 97%, 98%, 99% or 100% sequence identity to the amino acid sequence of SEQ ID NO: 384; or
kk) the VH has at least 91%, 92%, 93%, 94%, 95%, 96%, 97%, 98%, 99% or 100% sequence identity to the amino acid sequence of SEQ ID NO: 390; and the VL has at least 96%, 97%, 98%, 99% or 100% sequence identity to the amino acid sequence of SEQ ID NO: 394; or
ll) the VH has at least 93%, 94%, 95%, 96%, 97%, 98%, 99% or 100% sequence identity to the amino acid sequence of SEQ ID NO: 400; and the VL has at least 93%, 94%, 95%, 96%, 97%, 98%, 99% or 100% sequence identity to the amino acid sequence of SEQ ID NO: 404; or
mm) the VH has at least 92%, 93%, 94%, 95%, 96%, 97%, 98%, 99% or 100% sequence identity to the amino acid sequence of SEQ ID NO: 410; and the VL comprises the amino acid sequence of SEQ ID NO: 414; or
nn) the VH has at least 94%, 95%, 96%, 97%, 98%, 99% or 100% sequence identity to the amino acid sequence of SEQ ID NO: 420; and the VL has at 99% or 100% sequence identity to the amino acid sequence of SEQ ID NO: 424; or
oo) the VH has at least 96%, 97%, 98%, 99% or 100% sequence identity to the amino acid sequence of SEQ ID NO: 430; and the VL has at least 98%, 99% or 100% sequence identity to the amino acid sequence of SEQ ID NO: 434; or
pp) the VH has at least 93%, 94%, 95%, 96%, 97%, 98%, 99% or 100% sequence identity to the amino acid sequence of SEQ ID NO: 440; and the VL comprises the amino acid sequence of SEQ ID NO: 414; or
qq) the VH has at least 93%, 94%, 95%, 96%, 97%, 98%, 99% or 100% sequence identity to the amino acid sequence of SEQ ID NO: 450; and the VL has at least 99% or 100% sequence identity to the amino acid sequence of SEQ ID NO: 424; or
rr) the VH has at least 94%, 95%, 96%, 97%, 98%, 99% or 100% sequence identity to the amino acid sequence of SEQ ID NO: 460; and the VL has at least 99% or 100% sequence identity to the amino acid sequence of SEQ ID NO: 464; or
ss) the VH has at least 96%, 97%, 98%, 99% or 100% sequence identity to the amino acid sequence of SEQ ID NO: 470; and the VL has at least 99% or 100% sequence identity to the amino acid sequence of SEQ ID NO: 474; or
tt) the VH has at least 98%, 99% or 100% sequence identity to the amino acid sequence of SEQ ID NO: 480; and the VL comprises the amino acid sequence of SEQ ID NO: 484; or
uu) the VH has at 98%, 99% or 100% sequence identity to the amino acid sequence of SEQ ID NO: 490; and the VL has at least 99% or 100% sequence identity to the amino acid sequence of SEQ ID NO: 494; or
vv) the VH has at least 86%, 87%, 88%, 89%, 90%, 91%, 92%, 93%, 94%, 95%, 96%, 97%, 98%, 99% or 100% sequence identity to the amino acid sequence of SEQ ID NO: 500; and the VL has at least 97%, 98%, 99% or 100% sequence identity to the amino acid sequence of SEQ ID NO: 504; or
ww) the VH has at least 88%, 89%, 90%, 91%, 92%, 93%, 94%, 95%, 96%, 97%, 98%, 99% or 100% sequence identity to the amino acid sequence of SEQ ID NO: 510; and the VL has at least 98%, 99% or 100% sequence identity to the amino acid sequence of SEQ ID NO: 514; or
xx) the VH has at least 89%, 90%, 91%, 92%, 93%, 94%, 95%, 96%, 97%, 98%, 99% or 100% sequence identity to the amino acid sequence of SEQ ID NO: 520; and the VL has at least 99% or 100% sequence identity to the amino acid sequence of SEQ ID NO: 524; or
yy) the VH has at least 88%, 89%, 90%, 91%, 92%, 93%, 94%, 95%, 96%, 97%, 98%, 99% or 100% sequence identity to the amino acid sequence of SEQ ID NO: 530; and the VL comprises the amino acid sequence of SEQ ID NO: 534; or
zz) the VH has at least 91%, 92%, 93%, 94%, 95%, 96%, 97%, 98%, 99% or 100% sequence identity to the amino acid sequence of SEQ ID NO: 540; and the VL comprises the amino acid sequence of SEQ ID NO: 544; or
aaa) the VH has at least 89%, 90%, 91%, 92%, 93%, 94%, 95%, 96%, 97%, 98%, 99% or 100% sequence identity to the amino acid sequence of SEQ ID NO: 550; and the VL has at least 99% or 100% sequence identity to the amino acid sequence of SEQ ID NO: 554; or
bbb) the VH has at least 88%, 89%, 90%, 91%, 92%, 93%, 94%, 95%, 96%, 97%, 98%, 99% or 100% sequence identity to the amino acid sequence of SEQ ID NO: 560; and the VL has at least 98%, 99% or 100% sequence identity to the amino acid sequence of SEQ ID NO: 564; or
ccc) the VH has at least 92%, 93%, 94%, 95%, 96%, 97%, 98%, 99% or 100% sequence identity to the amino acid sequence of SEQ ID NO: 570; and the VL has at least 97%, 98%, 99% or 100% sequence identity to the amino acid sequence of SEQ ID NO: 574; or
ddd) the VH has at least 95%, 96%, 97%, 98%, 99% or 100% sequence identity to the amino acid sequence of SEQ ID NO: 580; and the VL has at least 99% or 100% sequence identity to the amino acid sequence of SEQ ID NO: 584; or
eee) the VH has at least 95%, 96%, 97%, 98%, 99% or 100% sequence identity to the amino acid sequence of SEQ ID NO: 590; and the VL has at least 99% or 100% sequence identity to the amino acid sequence of SEQ ID NO: 474; or
fff) the VH has at least 89%, 90%, 91%, 92%, 93%, 94%, 95%, 96%, 97%, 98%, 99% or 100% sequence identity to the amino acid sequence of SEQ ID NO: 600; and the VL has at least 99% or 100% sequence identity to the amino acid sequence of SEQ ID NO: 554; or
ggg) the VH has at least 92%, 93%, 94%, 95%, 96%, 97%, 98%, 99% or 100% sequence identity to the amino acid sequence of SEQ ID NO: 610; and the VL comprises the amino acid sequence of SEQ ID NO: 614; or
hhh) the VH has at least 96%, 97%, 98%, 99% or 100% sequence identity to the amino acid sequence of SEQ ID NO: 620; and the VL has at least 92%, 93%, 94%, 95%, 96%, 97%, 98%, 99% or 100% sequence identity to the amino acid sequence of SEQ ID NO: 624; or
iii) the VH has at 99% or 100% sequence identity to the amino acid sequence of SEQ ID NO: 630; and the VL has at least 99% or 100% sequence identity to the amino acid sequence of SEQ ID NO: 634; or
jjj) the VH has at least 96%, 97%, 98%, 99% or 100% sequence identity to the amino acid sequence of SEQ ID NO: 640; and the VL has at least 96%, 97%, 98%, 99% or 100% sequence identity to the amino acid sequence of SEQ ID NO: 644; or
kkk) the VH has at least 98%, 99% or 100% sequence identity to the amino acid sequence of SEQ ID NO: 650; and the VL has at least 99% or 100% sequence identity to the amino acid sequence of SEQ ID NO: 654; or
lll) the VH has at least 92%, 93%, 94%, 95%, 96%, 97%, 98%, 99% or 100% sequence identity to the amino acid sequence of SEQ ID NO: 660; and the VL has at least 99% or 100% sequence identity to the amino acid sequence of SEQ ID NO: 664; or
mmm) the VH has at least 92%, 93%, 94%, 95%, 96%, 97%, 98%, 99% or 100% sequence identity to the amino acid sequence of SEQ ID NO: 670; and the VL comprises the amino acid sequence of SEQ ID NO: 674; or
nnn) the VH has at least 97%, 98%, 99% or 100% sequence identity to the amino acid sequence of SEQ ID NO: 680; and the VL has at least 99% or 100% sequence identity to the amino acid sequence of SEQ ID NO: 684; or
ooo) the VH has at least 87%, 88%, 89%, 90%, 91%, 92%, 93%, 94%, 95%, 96%, 97%, 98%, 99% or 100% sequence identity to the amino acid sequence of SEQ ID NO: 690; and the VL has at least 96%, 97%, 98%, 99% or 100% sequence identity to the amino acid sequence of SEQ ID NO: 694; or
   ppp) the VH has at least 86%, 87%, 88%, 89%, 90%, 91%, 92%, 93%, 94%, 95%, 96%, 97%, 98%, 99% or 100% sequence identity to the amino acid sequence of SEQ ID NO: 700; and the VL has at least 94%, 95%, 96%, 97%, 98%, 99% or 100% sequence identity to the amino acid sequence of SEQ ID NO: 704; or
   qqq) the VH has at least 96%, 97%, 98%, 99% or 100% sequence identity to the amino acid sequence of SEQ ID NO: 710; and the VL has at 98%, 99% or 100% sequence identity to the amino acid sequence of SEQ ID NO: 714; or
   rrr) the VH comprises the amino acid sequence of SEQ ID NO: 720; and the VL has at least 95%, 96%, 97%, 98%, 99% or 100% sequence identity to the amino acid sequence of SEQ ID NO: 724; or
   sss) the VH has at least 97%, 98%, 99% or 100% sequence identity to the amino acid sequence of SEQ ID NO: 730; and the VL has at least 97%, 98%, 99% or 100% sequence identity to the amino acid sequence of SEQ ID NO: 734; or
   ttt) the VH has at least 88%, 89%, 90%, 91%, 92%, 93%, 94%, 95%, 96%, 97%, 98%, 99% or 100% sequence identity to the amino acid sequence of SEQ ID NO: 740; and the VL has at least 99% or 100% sequence identity to the amino acid sequence of SEQ ID NO: 744; or
   uuu) the VH has at least 99% or 100% sequence identity to the amino acid sequence of SEQ ID NO: 750; and the VL has at least 97%, 98%, 99% or 100% sequence identity to the amino acid sequence of SEQ ID NO: 754; or
   vvv) the VH has at least 97%, 98%, 99% or 100% sequence identity to the amino acid sequence of SEQ ID NO: 760; and the VL has at 98%, 99% or 100% sequence identity to the amino acid sequence of SEQ ID NO: 764; or
   www) the VH has at least 93%, 94%, 95%, 96%, 97%, 98%, 99% or 100% sequence identity to the amino acid sequence of SEQ ID NO: 770; and the VL has at least 97%, 98%, 99% or 100% sequence identity to the amino acid sequence of SEQ ID NO: 774; or
   xxx) the VH has at least 99% or 100% sequence identity to the amino acid sequence of SEQ ID NO: 750; and the VL has at least 96%, 97%, 98%, 99% or 100% sequence identity to the amino acid sequence of SEQ ID NO: 784; or
   yyy) the VH has at least 83%, 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 790; and the VL has at least 95%, 96%, 97%, 98%, 99% or 100% sequence identity to the amino acid sequence of SEQ ID NO: 794; or
   zzz) the VH has at least 92%, 93%, 94%, 95%, 96%, 97%, 98%, 99% or 100% sequence identity to the amino acid sequence of SEQ ID NO: 800; and the VL has at least 96%, 97%, 98%, 99% or 100% sequence identity to the amino acid sequence of SEQ ID NO: 804; or
   aaaa) the VH has at least 94%, 95%, 96%, 97%, 98%, 99% or 100% sequence identity to the amino acid sequence of SEQ ID NO: 810; and the VL has at least 99% or 100% sequence identity to the amino acid sequence of SEQ ID NO: 814; or
   bbbb) the VH has at least 98%, 99% or 100% sequence identity to the amino acid sequence of SEQ ID NO: 820; and the VL has at least 98%, 99% or 100% sequence identity to the amino acid sequence of SEQ ID NO: 824; or
   cccc) the VH has at least 94%, 95%, 96%, 97%, 98%, 99% or 100% sequence identity to the amino acid sequence of SEQ ID NO: 830; and the VL comprises the amino acid sequence of SEQ ID NO: 834; or
   dddd) the VH has at least 90%, 91%, 92%, 93%, 94%, 95%, 96%, 97%, 98%, 99% or 100% sequence identity to the amino acid sequence of SEQ ID NO: 840; and the VL comprises the amino acid sequence of SEQ ID NO: 844.

In some embodiments of the invention, the biparatopic antibody, or a functional fragment thereof comprises:
a) a first binding site comprising variable regions VH and/or VL respectively, set forth in SEQ ID NO: 10 and SEQ ID NO: 14; SEQ ID NO: 30 and SEQ ID NO: 84; SEQ ID NO: 50 and SEQ ID NO: 54; SEQ ID NO: 90 and SEQ ID NO: 94; SEQ ID NO: 150 and SEQ ID NO: 154; SEQ ID NO: 180 and SEQ ID NO: 184; SEQ ID NO: 690 and SEQ ID NO: 694; SEQ ID NO: 670 and SEQ ID NO: 674; SEQ ID NO: 320 and SEQ ID NO: 324; SEQ ID NO: 360 and SEQ ID NO: 364; SEQ ID NO: 400 and SEQ ID NO: 404; SEQ ID NO: 530 and SEQ ID NO: 534; SEQ ID NO: 460 and SEQ ID NO: 464; SEQ ID NO: 590 and SEQ ID NO: 474; SEQ ID NO: 570 and SEQ ID NO: 574; SEQ ID NO: 340 and SEQ ID NO: 344; SEQ ID NO: 510 and SEQ ID NO: 514; SEQ ID NO: 330 and SEQ ID NO: 334; SEQ ID NO: 610 and SEQ ID NO: 614 and
b) a second binding site comprising variable regions VH and/or VL respectively, set forth in SEQ ID NO: 10 and SEQ ID NO: 14; SEQ ID NO: 30 and SEQ ID NO: 84 SEQ ID NO: 50 and SEQ ID NO: 54; SEQ ID NO: 90 and SEQ ID NO: 94; SEQ ID NO: 150 and SEQ ID NO: 154; SEQ ID NO: 180 and SEQ ID NO: 184; SEQ ID NO: 690 and SEQ ID NO: 694; SEQ ID NO: 670 and SEQ ID NO: 674; SEQ ID NO: 320 and SEQ ID NO: 324; SEQ ID NO: 360 and SEQ ID NO: 364; SEQ ID NO: 400 and SEQ ID NO: 404; SEQ ID NO: 530 and SEQ ID NO: 534; SEQ ID NO: 460 and SEQ ID NO: 464; SEQ ID NO: 590 and SEQ ID NO: 474; SEQ ID NO: 570 and SEQ ID NO: 574; SEQ ID NO: 340 and SEQ ID NO: 344; SEQ ID NO: 510 and SEQ ID NO: 514; SEQ ID NO: 330 and SEQ ID NO: 334; SEQ ID NO: 610 and SEQ ID NO: 614;
c) a first binding site as set in (a) and a second binding site as set in (b) not identic to the first binding site.

In particular in some embodiments of the invention, the biparatopic antibody, or a functional fragment thereof comprises:
a) a first binding site comprising variable regions VH and/or VL respectively, set forth in SEQ ID NO: 10 and SEQ ID NO: 14;
b) a second binding site comprising variable regions VH and/or VL respectively, set forth in SEQ ID NO: 50 and SEQ ID NO: 54;
c) a first binding site as in (a) and a second binding site as in (b); or
d) a first binding site comprising variable regions VH and/or VL respectively, set forth in SEQ ID NO: 10 and SEQ ID NO: 14;
e) a second binding site comprising variable regions VH and/or VL respectively, set forth in SEQ ID NO: 90 and SEQ ID NO: 94;
f) a first binding site as in (d) and a second binding site as in (e);
g) a first binding site comprising variable regions VH and/or VL respectively, set forth in SEQ ID NO: 10 and SEQ ID NO 14;
h) a second binding site comprising variable regions VH and/or VL respectively, set forth in SEQ ID NO: 30 and SEQ ID NO: 84;
i) a first binding site as in (g) and a second binding site as in (h);
j) a first binding site comprising variable regions VH and/or VL respectively, set forth in SEQ ID NO: 50 and SEQ ID NO: 54;
k) a second binding site comprising variable regions VH and/or VL respectively, set forth in SEQ ID NO: 90 and SEQ ID NO: 94;
l) a first binding site as in (j) and a second binding site as in (k);
m) a first binding site comprising variable regions VH and/or VL respectively, set forth in SEQ ID NO: 50 and SEQ ID NO: 54;
n) a second binding site comprising variable regions VH and/or VL respectively, set forth in SEQ ID NO: 30 and SEQ ID NO: 84;
o) a first binding site as in (m) and a second binding site as in (n);
p) a first binding site comprising variable regions VH and/or VL respectively, set forth in SEQ ID NO: 180 and SEQ ID NO: 184;
q) a second binding site comprising variable regions VH and/or VL respectively, set forth in SEQ ID NO: 10 and SEQ ID NO: 14;
r) a first binding site as in (p) and a second binding site as in (q);
s) a first binding site comprising variable regions VH and/or VL respectively, set forth in SEQ ID NO: 150 and SEQ ID NO: 154;
t) a second binding site comprising variable regions VH and/or VL respectively, set forth in SEQ ID NO: 10 and SEQ ID NO: 14;
u) a first binding site as in (s) and a second binding site as in (t);
v) a first binding site comprising a variable regions VH and/or VL respectively, set forth in SEQ ID NO: 180 and SEQ ID NO: 184;
w) a second binding site comprising variable regions VH and/or VL respectively, set forth in SEQ ID NO: 90 and SEQ ID NO: 94;
x) a first binding site as in (v) and a second binding site as in (w); or
y) a first binding site comprising variable regions VH and/or VL respectively, set forth in SEQ ID NO: 150 and SEQ ID NO: 154;
z) a second binding site comprising variable regions VH and/or VL respectively, set forth in SEQ ID NO: 90 and SEQ ID NO: 94;
aa) a first binding site as in (y) and a second binding site as in (z); or bb) a first binding site comprising variable regions VH and/or VL respectively, set forth in SEQ ID NO: 30 and SEQ ID NO: 84;
cc) a second binding site comprising variable regions VH and/or VL respectively, set forth in SEQ ID NO: 690 and SEQ ID NO: 694;
dd) a first binding site as in (bb) and a second binding site as in (cc); or ee) a first binding site comprising variable regions VH and/or VL respectively, set forth in SEQ ID NO: 690 and SEQ ID NO: 694;
ff) a second binding site comprising variable regions VH and/or VL respectively, set forth in SEQ ID NO: 670 and SEQ ID NO: 674;
gg) a first binding site as in (ee) and a second binding site as in (ff); or hh) a first binding site comprising variable regions VH and/or VL respectively, set forth in SEQ ID NO: 320 and SEQ ID NO: 324;
ii) a second binding site comprising variable regions VH and/or VL respectively, set forth in SEQ ID NO: 690 and SEQ ID NO: 694;
jj) a first binding site as in (hh) and a second binding site as in (ii); or
kk) a first binding site comprising variable regions VH and/or VL respectively, set forth in SEQ ID NO: 670 and SEQ ID NO: 674;
ll) a second binding site comprising variable regions VH and/or VL respectively, set forth in SEQ ID NO: 690 and SEQ ID NO: 694;
mm) a first binding site as in (kk) and a second binding site as in (ll); or
nn) a first binding site comprising variable regions VH and/or VL respectively, set forth in SEQ ID NO: 50 and SEQ ID NO: 54;
oo) a second binding site comprising variable regions VH and/or VL respectively, set forth in SEQ ID NO: 360 and SEQ ID NO: 364;
pp) a first binding site as in (nn) and a second binding site as in (oo); or
qq) a first binding site comprising variable regions VH and/or VL respectively, set forth in SEQ ID NO: 400 and SEQ ID NO: 404;
rr) a second binding site comprising variable regions VH and/or VL respectively, set forth in SEQ ID NO: 90 and SEQ ID NO: 94;
ss) a first binding site as in (qq) and a second binding site as in (rr); or
tt) a first binding site comprising variable regions VH and/or VL respectively, set forth in SEQ ID NO: 530 and SEQ ID NO: 534;
uu) a second binding site comprising variable regions VH and/or VL respectively, set forth in SEQ ID NO: 460 and SEQ ID NO: 464;
vv) a first binding site as in (tt) and a second binding site as in (uu); or
ww) a first binding site comprising variable regions VH and/or VL respectively, set forth in SEQ ID NO: 10 and SEQ ID NO: 14;
xx) a second binding site comprising variable regions VH and/or VL respectively, set forth in SEQ ID NO: 530 and SEQ ID NO: 534;
yy) a first binding site as in (ww) and a second binding site as in (xx); or
zz) a first binding site comprising variable regions VH and/or VL respectively, set forth in SEQ ID NO: 460 and SEQ ID NO: 464;
aaa) a second binding site comprising variable regions VH and/or VL respectively, set forth in SEQ ID NO: 150 and SEQ ID NO: 154;
bbb) a first binding site as in (zz) and a second binding site as in (aaa); or
ccc) a first binding site comprising variable regions VH and/or VL respectively, set forth in SEQ ID NO: 460 and SEQ ID NO: 464;
ddd) a second binding site comprising variable regions VH and/or VL respectively, set forth in SEQ ID NO: 690 and SEQ ID NO: 694;
eee) a first binding site as in (ccc) and a second binding site as in (ddd); or
fff) a first binding site comprising variable regions VH and/or VL respectively, set forth in SEQ ID NO: 530 and SEQ ID NO: 534;
ggg) a second binding site comprising variable regions VH and/or VL respectively, set forth in SEQ ID NO: 400 and SEQ ID NO: 404;
hhh) a first binding site as in (fff) and a second binding site as in (ggg); or
iii) a first binding site comprising variable regions VH and/or VL respectively, set forth in SEQ ID NO: 320 and SEQ ID NO: 324;
jjj) a second binding site comprising variable regions VH and/or VL respectively, set forth in SEQ ID NO: 460 and SEQ ID NO: 464;
kkk) a first binding site as in (iii) and a second binding site as in (jjj); or
lll) a first binding site comprising variable regions VH and/or VL respectively, set forth in SEQ ID NO: 50 and SEQ ID NO: 54;
mmm) a second binding site comprising variable regions VH and/or VL respectively, set forth in SEQ ID NO: 460 and SEQ ID NO: 464;
nnn) a first binding site as in (lll) and a second binding site as in (mmm); or
ooo) a first binding site comprising variable regions VH and/or VL respectively, set forth in SEQ ID NO: 670 and SEQ ID NO: 674;
ppp) a second binding site comprising variable regions VH and/or VL respectively, set forth in SEQ ID NO: 460 and SEQ ID NO: 464;
qqq) a first binding site as in (ooo) and a second binding site as in (ppp); or
rrr) a first binding site comprising variable regions VH and/or VL respectively, set forth in SEQ ID NO: 10 and SEQ ID NO: 14;
sss) a second binding site comprising variable regions VH and/or VL respectively, set forth in SEQ ID NO: 460 and SEQ ID NO: 464;
ttt) a first binding site as in (rrr) and a second binding site as in (sss); or
uuu) a first binding site comprising variable regions VH and/or VL respectively, set forth in SEQ ID NO: 590 and SEQ ID NO: 474;
vvv) a second binding site comprising variable regions VH and/or VL respectively, set forth in SEQ ID NO: 10 and SEQ ID NO: 14;
www) a first binding site as in (uuu) and a second binding site as in (vvv); or
xxx) a first binding site comprising variable regions VH and/or VL respectively, set forth in SEQ ID NO: 590 and SEQ ID NO: 474;
yyy) a second binding site comprising variable regions VH and/or VL respectively, set forth in SEQ ID NO: 400 and SEQ ID NO: 404;
zzz) a first binding site as in (xxx) and a second binding site as in (yyy); or
aaaa) a first binding site comprising variable regions VH and/or VL respectively, set forth in SEQ ID NO: 590 and SEQ ID NO: 474;
bbbb) a second binding site comprising variable regions VH and/or VL respectively, set forth in SEQ ID NO: 320 and SEQ ID NO: 324;
cccc) a first binding site as in (aaaa) and a second binding site as in (bbbb); or
dddd) a first binding site comprising variable regions VH and/or VL respectively, set forth in SEQ ID NO: 590 and SEQ ID NO: 474;
eeee) a second binding site comprising variable regions VH and/or VL respectively, set forth in SEQ ID NO: 570 and SEQ ID NO: 574;
ffff) a first binding site as in (dddd) and a second binding site as in (eeee); or
gggg) a first binding site comprising variable regions VH and/or VL respectively, set forth in SEQ ID NO: 590 and SEQ ID NO: 474;
hhhh) a second binding site comprising variable regions VH and/or VL respectively, set forth in SEQ ID NO: 340 and SEQ ID NO: 344;
iiii) a first binding site as in (gggg) and a second binding site as in (hhhh); or
jjjj) a first binding site comprising variable regions VH and/or VL respectively, set forth in SEQ ID NO: 30 and SEQ ID NO: 84;
kkkk) a second binding site comprising variable regions VH and/or VL respectively, set forth in SEQ ID NO: 590 and SEQ ID NO: 474;
llll) a first binding site as in (jjjj) and a second binding site as in (kkkk); or
mmmm) a first binding site comprising variable regions VH and/or VL respectively, set forth in SEQ ID NO: 30 and SEQ ID NO: 84;
nnnn) a second binding site comprising variable regions VH and/or VL respectively, set forth in SEQ ID NO: 510 and SEQ ID NO: 514;
oooo) a first binding site as in (mmmm) and a second binding site as in (nnnn); or
pppp) a first binding site comprising variable regions VH and/or VL respectively, set forth in SEQ ID NO: 340 and SEQ ID NO: 344;
qqqq) a second binding site comprising variable regions VH and/or VL respectively, set forth in SEQ ID NO: 690 and SEQ ID NO: 694;
rrrr) a first binding site as in (pppp) and a second binding site as in (qqqq); or
ssss) a first binding site comprising variable regions VH and/or VL respectively, set forth in SEQ ID NO: 10 and SEQ ID NO: 14;
tttt) a second binding site comprising variable regions VH and/or VL respectively, set forth in SEQ ID NO: 400 and SEQ ID NO: 404;
uuuu) a first binding site as in (ssss) and a second binding site as in (tttt); or
vvvv) a first binding site comprising variable regions VH and/or VL respectively, set forth in SEQ ID NO: 690 and SEQ ID NO: 694;
wwww) a second binding site comprising variable regions VH and/or VL respectively, set forth in SEQ ID NO: 10 and SEQ ID NO: 14;
xxxx) a first binding site as in (vvvv) and a second binding site as in (wwww); or
yyyy) a first binding site comprising variable regions VH and/or VL respectively, set forth in SEQ ID NO: 10 and SEQ ID NO: 14;
zzzz) a second binding site comprising variable regions VH and/or VL respectively, set forth in SEQ ID NO: 330 and SEQ ID NO: 334;
aaaaa) a first binding site as in (yyyy) and a second binding site as in (zzzz); or
bbbbb) a first binding site comprising variable regions VH and/or VL respectively, set forth in SEQ ID NO: 670 and SEQ ID NO: 674;
ccccc) a second binding site comprising variable regions VH and/or VL respectively, set forth in SEQ ID NO: 10 and SEQ ID NO: 14;
ddddd) a first binding site as in (bbbbb) and a second binding site as in (ccccc); or
eeeee) a first binding site comprising variable regions VH and/or VL respectively, set forth in SEQ ID NO: 10 and SEQ ID NO: 14;
fffff) a second binding site comprising variable regions VH and/or VL respectively, set forth in SEQ ID NO: 610 and SEQ ID NO: 614;
ggggg) a first binding site as in (eeeee) and a second binding site as in (fffff).

In some embodiments, the biparatopic antibody or a functional fragment thereof comprises a first binding site and a second distinct binding site selected from the group consisting of binding sites comprising:
a) VH-CDR1 comprising the amino acid sequence of SEQ ID NO: 11; VH-CDR2 comprising the amino acid sequence of SEQ ID NO: 12; VH-CDR3 comprising the amino acid sequence of SEQ ID NO: 13; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 17; or
b) VH-CDR1 comprising the amino acid sequence of SEQ ID NO: 21; VH-CDR2 comprising the amino acid sequence of SEQ ID NO: 22; VH-CDR3 comprising the amino acid sequence YSY; VL-CDR1 comprising the amino acid sequence of SEQ ID NO: 25; VL-CDR2 comprising the amino acid sequence of SEQ ID NO: 26; and VL-CDR3 comprising the amino acid sequence of SEQ ID NO: 27; or
c) VH-CDR1 comprising the amino acid sequence of SEQ ID NO: 31; VH-CDR2 comprising the amino acid sequence of SEQ ID NO: 32; VH-CDR3 comprising the amino acid sequence of SEQ ID NO: 33; VL-CDR1 comprising the amino acid sequence of SEQ ID NO: 35; VL-CDR2 comprising the amino acid sequence of SEQ ID NO: 36; and VL-CDR3 comprising the amino acid sequence of SEQ ID NO: 37; or
d) VH-CDR1 comprising the amino acid sequence of SEQ ID NO: 41; VH-CDR2 comprising the amino acid sequence of SEQ ID NO: 42; VH-CDR3 comprising the amino acid sequence of SEQ ID NO: 43; VL-CDR1 comprising the amino acid sequence of SEQ ID NO: 45; VL-CDR2 comprising the amino acid sequence of SEQ ID NO: 46; and VL-CDR3 comprising the amino acid sequence of SEQ ID NO: 47; or
e) VH-CDR1 comprising the amino acid sequence of SEQ ID NO: 21; VH-CDR2 comprising the amino acid sequence of SEQ ID NO: 52; VH-CDR3 comprising the amino acid sequence YSF; VL-CDR1 comprising the amino acid sequence of SEQ ID NO: 55; VL-CDR2 comprising the amino acid sequence of SEQ ID NO: 56; and VL-CDR3 comprising the amino acid sequence of SEQ ID NO: 27; or
f) VH-CDR1 comprising the amino acid sequence of SEQ ID NO: 61; VH-CDR2 comprising the amino acid sequence of SEQ ID NO: 62; VH-CDR3 comprising the amino acid sequence of SEQ ID NO: 43; VL-CDR1 comprising the amino acid sequence of SEQ ID NO: 65; VL-CDR2 comprising the amino acid sequence of SEQ ID NO: 46; and VL-CDR3 comprising the amino acid sequence of SEQ ID NO: 67; or
g) VH-CDR1 comprising the amino acid sequence of SEQ ID NO: 21; VH-CDR2 comprising the amino acid sequence of SEQ ID NO: 72; VH-CDR3 comprising the amino acid sequence YSY; VL-CDR1 comprising the amino acid sequence of SEQ ID NO: 75; VL-CDR2 comprising the amino acid sequence of SEQ ID NO: 76; and VL-CDR3 comprising the amino acid sequence of SEQ ID NO: 77; or
h) VH-CDR1 comprising the amino acid sequence of SEQ ID NO: 31; VH-CDR2 comprising the amino acid sequence of SEQ ID NO: 32; VH-CDR3 comprising the amino acid sequence of SEQ ID NO: 33; VL-CDR1 comprising the amino acid sequence of SEQ ID NO: 85; VL-CDR2 comprising the amino acid sequence of SEQ ID NO: 36; and VL-CDR3 comprising the amino acid sequence of SEQ ID NO: 87; or
i) VH-CDR1 comprising the amino acid sequence of SEQ ID NO: 91; VH-CDR2 comprising the amino acid sequence of SEQ ID NO: 92; VH-CDR3 comprising the amino acid sequence of SEQ ID NO: 93; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 97; or
j) VH-CDR1 comprising the amino acid sequence of SEQ ID NO: 101; VH-CDR2 comprising the amino acid sequence of SEQ ID NO: 102; VH-CDR3 comprising the amino acid sequence of SEQ ID NO: 103; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107; or
k) VH-CDR1 comprising the amino acid sequence of SEQ ID NO: 111; VH-CDR2 comprising the amino acid sequence of SEQ ID NO: 112; VH-CDR3 comprising the amino acid sequence of SEQ ID NO: 113; VL-CDR1 comprising the amino acid sequence of SEQ ID NO: 115; VL-CDR2 comprising the amino acid sequence of SEQ ID NO: 106; and VL-CDR3 comprising the amino acid sequence of SEQ ID NO: 117; or
l) VH-CDR1 comprising the amino acid sequence of SEQ ID NO: 281; VH-CDR2 comprising the amino acid sequence of SEQ ID NO: 282; VH-CDR3 comprising the amino acid sequence of SEQ ID NO: 283; VL-CDR1 comprising the amino acid sequence of SEQ ID NO: 285; VL-CDR2 comprising the amino acid sequence of SEQ ID NO: 286; and VL-CDR3 comprising the amino acid sequence of SEQ ID NO: 287; or
m) VH-CDR1 comprising the amino acid sequence of SEQ ID NO: 31; VH-CDR2 comprising the amino acid sequence of SEQ ID NO: 192; VH-CDR3 comprising the amino acid sequence of SEQ ID NO: 193; VL-CDR1 comprising the amino acid sequence of SEQ ID NO: 195; VL-CDR2 comprising the amino acid sequence of SEQ ID NO: 96; and VL-CDR3 comprising the amino acid sequence of SEQ ID NO: 197; or
n) VH-CDR1 comprising the amino acid sequence of SEQ ID NO: 141; VH-CDR2 comprising the amino acid sequence of SEQ ID NO: 142; VH-CDR3 comprising the amino acid sequence of SEQ ID NO: 143; VL-CDR1 comprising the amino acid sequence of SEQ ID NO: 145; VL-CDR2 comprising the amino acid sequence of SEQ ID NO: 16; and VL-CDR3 comprising the amino acid sequence of SEQ ID NO: 17; or
o) VH-CDR1 comprising the amino acid sequence of SEQ ID NO: 151; VH-CDR2 comprising the amino acid sequence of SEQ ID NO: 152; VH-CDR3 comprising the amino acid sequence of SEQ ID NO: 153; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107; or
p) VH-CDR1 comprising the amino acid sequence of SEQ ID NO: 161; VH-CDR2 comprising the amino acid sequence of SEQ ID NO: 162; VH-CDR3 comprising the amino acid sequence of SEQ ID NO: 163; VL-CDR1 comprising the amino acid sequence of SEQ ID NO: 165; VL-CDR2 comprising the amino acid sequence of SEQ ID NO: 16; and VL-CDR3 comprising the amino acid sequence of SEQ ID NO: 167; or
q) VH-CDR1 comprising the amino acid sequence of SEQ ID NO: 171; VH-CDR2 comprising the amino acid sequence of SEQ ID NO: 172; VH-CDR3 comprising the amino acid sequence of SEQ ID NO: 173; VL-CDR1 comprising the amino acid sequence of SEQ ID NO: 175; VL-CDR2 comprising the amino acid sequence of SEQ ID NO: 176; and VL-CDR3 comprising the amino acid sequence of SEQ ID NO: 177; or
r) VH-CDR1 comprising the amino acid sequence of SEQ ID NO: 181; VH-CDR2 comprising the amino acid sequence of SEQ ID NO: 182; VH-CDR3 comprising the amino acid sequence of SEQ ID NO: 183; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 187; or
s) VH-CDR1 comprising the amino acid sequence of SEQ ID NO: 201; VH-CDR2 comprising the amino acid sequence of SEQ ID NO: 202; VH-CDR3 comprising the amino acid sequence of SEQ ID NO: 153; VL-CDR1 comprising the amino acid sequence of SEQ ID NO: 105; VL-CDR2 comprising the amino acid sequence of SEQ ID NO: 206; and VL-CDR3 comprising the amino acid sequence of SEQ ID NO: 107; or
t) VH-CDR1 comprising the amino acid sequence of SEQ ID NO: 211; VH-CDR2 comprising the amino acid sequence of SEQ ID NO: 212; VH-CDR3 comprising the amino acid sequence of SEQ ID NO: 213; VL-CDR1 comprising the amino acid sequence of SEQ ID NO: 215; VL-CDR2 comprising the amino acid sequence of SEQ ID NO: 216; and VL-CDR3 comprising the amino acid sequence of SEQ ID NO: 217; or
u) VH-CDR1 comprising the amino acid sequence of SEQ ID NO: 31; VH-CDR2 comprising the amino acid sequence of SEQ ID NO: 222; VH-CDR3 comprising the amino acid sequence of SEQ ID NO: 223; VL-CDR1 comprising the amino acid sequence of SEQ ID NO: 225; VL-CDR2 comprising the amino acid sequence of SEQ ID NO: 96; and VL-CDR3 comprising the amino acid sequence of SEQ ID NO: 227; or
v) VH-CDR1 comprising the amino acid sequence of SEQ ID NO: 231; VH-CDR2 comprising the amino acid sequence of SEQ ID NO: 232; VH-CDR3 comprising the amino acid sequence of SEQ ID NO: 233; VL-CDR1 comprising the amino acid sequence of SEQ ID NO: 235; VL-CDR2 comprising the amino acid sequence of SEQ ID NO: 236; and VL-CDR3 comprising the amino acid sequence of SEQ ID NO: 237; or
w) VH-CDR1 comprising the amino acid sequence of SEQ ID NO: 31; VH-CDR2 comprising the amino acid sequence of SEQ ID NO: 242; VH-CDR3 comprising the amino acid sequence of SEQ ID NO: 243; VL-CDR1 comprising the amino acid sequence of SEQ ID NO: 225; VL-CDR2 comprising the amino acid sequence of SEQ ID NO: 96; and VL-CDR3 comprising the amino acid sequence of SEQ ID NO: 247; or
x) VH-CDR1 comprising the amino acid sequence of SEQ ID NO: 31; VH-CDR2 comprising the amino acid sequence of SEQ ID NO: 252; VH-CDR3 comprising the amino acid sequence of SEQ ID NO: 253; VL-CDR1 comprising the amino acid sequence of SEQ ID NO: 255; VL-CDR2 comprising the amino acid sequence of SEQ ID NO: 256; and VL-CDR3 comprising the amino acid sequence of SEQ ID NO: 257; or
y) VH-CDR1 comprising the amino acid sequence of SEQ ID NO: 261; VH-CDR2 comprising the amino acid sequence of SEQ ID NO: 262; VH-CDR3 comprising the amino acid sequence of SEQ ID NO: 263; VL-CDR1 comprising the amino acid sequence of SEQ ID NO: 265; VL-CDR2 comprising the amino acid sequence of SEQ ID NO: 176; and VL-CDR3 comprising the amino acid sequence of SEQ ID NO: 267; or
z) VH-CDR1 comprising the amino acid sequence of SEQ ID NO: 271; VH-CDR2 comprising the amino acid sequence of SEQ ID NO: 272; VH-CDR3 comprising the amino acid sequence of SEQ ID NO: 273; VL-CDR1 comprising the amino acid sequence of SEQ ID NO: 275; VL-CDR2 comprising the amino acid sequence of SEQ ID NO: 276; and VL-CDR3 comprising the amino acid sequence of SEQ ID NO: 277; or
aa) VH-CDR1 comprising the amino acid sequence of SEQ ID NO: 301; VH-CDR2 comprising the amino acid sequence of SEQ ID NO: 302; VH-CDR3 comprising the amino acid sequence of SEQ ID NO: 303; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 307; or
bb) VH-CDR1 comprising the amino acid sequence of SEQ ID NO: 311; VH-CDR2 comprising the amino acid sequence of SEQ ID NO: 312; VH-CDR3 comprising the amino acid sequence of SEQ ID NO: 313; VL-CDR1 comprising the amino acid sequence of SEQ ID NO: 315; VL-CDR2 comprising the amino acid sequence of SEQ ID NO: 46; and VL-CDR3 comprising the amino acid sequence of SEQ ID NO: 67; or
cc) VH-CDR1 comprising the amino acid sequence of SEQ ID NO: 321; VH-CDR2 comprising the amino acid sequence of SEQ ID NO: 322; VH-CDR3 comprising the amino acid sequence of SEQ ID NO: 323; VL-CDR1 comprising the amino acid sequence of SEQ ID NO: 325; VL-CDR2 comprising the amino acid sequence of SEQ ID NO: 326; and VL-CDR3 comprising the amino acid sequence of SEQ ID NO: 327; or
dd) VH-CDR1 comprising the amino acid sequence of SEQ ID NO: 151; VH-CDR2 comprising the amino acid sequence of SEQ ID NO: 332; VH-CDR3 comprising the amino acid sequence of SEQ ID NO: 333; VL-CDR1 comprising the amino acid sequence of SEQ ID NO: 335; VL-CDR2 comprising the amino acid sequence of SEQ ID NO: 336; and VL-CDR3 comprising the amino acid sequence of SEQ ID NO: 107; or
ee) VH-CDR1 comprising the amino acid sequence of SEQ ID NO: 341; VH-CDR2 comprising the amino acid sequence of SEQ ID NO: 342; VH-CDR3 comprising the amino acid sequence of SEQ ID NO: 343; VL-CDR1 comprising the amino acid sequence of SEQ ID NO: 345; VL-CDR2 comprising the amino acid sequence of SEQ ID NO: 346; and VL-CDR3 comprising the amino acid sequence of SEQ ID NO: 347; or
ff) VH-CDR1 comprising the amino acid sequence of SEQ ID NO: 351; VH-CDR2 comprising the amino acid sequence of SEQ ID NO: 352; VH-CDR3 comprising the amino acid sequence of SEQ ID NO: 353; VL-CDR1 comprising the amino acid sequence of SEQ ID NO: 355; VL-CDR2 comprising the amino acid sequence of SEQ ID NO: 356; and VL-CDR3 comprising the amino acid sequence of SEQ ID NO: 357; or
gg) VH-CDR1 comprising the amino acid sequence of SEQ ID NO: 361; VH-CDR2 comprising the amino acid sequence of SEQ ID NO: 362; VH-CDR3 comprising the amino acid sequence of SEQ ID NO: 363; VL-CDR1 comprising the amino acid sequence of SEQ ID NO: 365; VL-CDR2 comprising the amino acid sequence of SEQ ID NO: 16; and VL-CDR3 comprising the amino acid sequence of SEQ ID NO: 367; or
hh) VH-CDR1 comprising the amino acid sequence of SEQ ID NO: 371; VH-CDR2 comprising the amino acid sequence of SEQ ID NO: 372; VH-CDR3 comprising the amino acid sequence of SEQ ID NO: 373; VL-CDR1 comprising the amino acid sequence of SEQ ID NO: 345; VL-CDR2 comprising the amino acid sequence of SEQ ID NO: 376; and VL-CDR3 comprising the amino acid sequence of SEQ ID NO: 347; or
ii) VH-CDR1 comprising the amino acid sequence of SEQ ID NO: 351; VH-CDR2 comprising the amino acid sequence of SEQ ID NO: 382; VH-CDR3 comprising the amino acid sequence of SEQ ID NO: 383; VL-CDR1 comprising the amino acid sequence of SEQ ID NO: 385; VL-CDR2 comprising the amino acid sequence of SEQ ID NO: 386; and VL-CDR3 comprising the amino acid sequence of SEQ ID NO: 387; or
jj) VH-CDR1 comprising the amino acid sequence of SEQ ID NO: 351; VH-CDR2 comprising the amino acid sequence of SEQ ID NO: 382; VH-CDR3 comprising the amino acid sequence of SEQ ID NO: 393; VL-CDR1 comprising the amino acid sequence of SEQ ID NO: 395; VL-CDR2 comprising the amino acid sequence of SEQ ID NO: 356; and VL-CDR3 comprising the amino acid sequence of SEQ ID NO: 357; or
kk) VH-CDR1 comprising the amino acid sequence of SEQ ID NO: 351; VH-CDR2 comprising the amino acid sequence of SEQ ID NO: 382; VH-CDR3 comprising the amino acid sequence of SEQ ID NO: 393; VL-CDR1 comprising the amino acid sequence of SEQ ID NO: 405; VL-CDR2 comprising the amino acid sequence of SEQ ID NO: 356; and VL-CDR3 comprising the amino acid sequence of SEQ ID NO: 357; or
ll) VH-CDR1 comprising the amino acid sequence of SEQ ID NO: 411; VH-CDR2 comprising the amino acid sequence of SEQ ID NO: 412; VH-CDR3 comprising the amino acid sequence of SEQ ID NO: 413; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107; or
mm) VH-CDR1 comprising the amino acid sequence of SEQ ID NO: 421; VH-CDR2 comprising the amino acid sequence of SEQ ID NO: 422; VH-CDR3 comprising the amino acid sequence GNY; VL-CDR1 comprising the amino acid sequence of SEQ ID NO: 425; VL-CDR2 comprising the amino acid sequence of SEQ ID NO: 426; and VL-CDR3 comprising the amino acid sequence of SEQ ID NO: 427; or
nn) VH-CDR1 comprising the amino acid sequence of SEQ ID NO: 431; VH-CDR2 comprising the amino acid sequence of SEQ ID NO: 432; VH-CDR3 comprising the amino acid sequence of SEQ ID NO: 433; VL-CDR1 comprising the amino acid sequence of SEQ ID NO: 435; VL-CDR2 comprising the amino acid sequence of SEQ ID NO: 436; and VL-CDR3 comprising the amino acid sequence of SEQ ID NO: 477; or
oo) VH-CDR1 comprising the amino acid sequence of SEQ ID NO: 151; VH-CDR2 comprising the amino acid sequence of SEQ ID NO: 442; VH-CDR3 comprising the amino acid sequence of SEQ ID NO: 443; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107; or
pp) CDR1 comprising the amino acid sequence of SEQ ID NO: 461; VH-CDR2 comprising the amino acid sequence of SEQ ID NO: 462; VH-CDR3 comprising the amino acid sequence of SEQ ID NO: 463; VL-CDR1 comprising the amino acid sequence of SEQ ID NO: 465; VL-CDR2 comprising the amino acid sequence of SEQ ID NO: 16; and VL-CDR3 comprising the amino acid sequence of SEQ ID NO: 467; or
qq) CDR1 comprising the amino acid sequence of SEQ ID NO: 141; VH-CDR2 comprising the amino acid sequence of SEQ ID NO: 472; VH-CDR3 comprising the amino acid sequence of SEQ ID NO: 473; VL-CDR1 comprising the amino acid sequence of SEQ ID NO: 475; VL-CDR2 comprising the amino acid sequence of SEQ ID NO: 476; and VL-CDR3 comprising the amino acid sequence of SEQ ID NO: 477; or
rr) CDR1 comprising the amino acid sequence of SEQ ID NO: 481; VH-CDR2 comprising the amino acid sequence of SEQ ID NO: 482; VH-CDR3 comprising the amino acid sequence of SEQ ID NO: 483; VL-CDR1 comprising the amino acid sequence of SEQ ID NO: 165; VL-CDR2 comprising the amino acid sequence of SEQ ID NO: 16; and VL-CDR3 comprising the amino acid sequence of SEQ ID NO: 487; or
ss) CDR1 comprising the amino acid sequence of SEQ ID NO: 141; VH-CDR2 comprising the amino acid sequence of SEQ ID NO: 492; VH-CDR3 comprising the amino acid sequence of SEQ ID NO: 493; VL-CDR1 comprising the amino acid sequence of SEQ ID NO: 495; VL-CDR2 comprising the amino acid sequence of SEQ ID NO: 496; and VL-CDR3 comprising the amino acid sequence of SEQ ID NO: 497; or
tt) CDR1 comprising the amino acid sequence of SEQ ID NO: 151; VH-CDR2 comprising the amino acid sequence of SEQ ID NO: 502; VH-CDR3 comprising the amino acid sequence of SEQ ID NO: 503; VL-CDR1 comprising the amino acid sequence of SEQ ID NO: 105; VL-CDR2 comprising the amino acid sequence of SEQ ID NO: 336; and VL-CDR3 comprising the amino acid sequence of SEQ ID NO: 107; or
uu) CDR1 comprising the amino acid sequence of SEQ ID NO: 311; VH-CDR2 comprising the amino acid sequence of SEQ ID NO: 512; VH-CDR3 comprising the amino acid sequence of SEQ ID NO: 513; VL-CDR1 comprising the amino acid sequence of SEQ ID NO: 515; VL-CDR2 comprising the amino acid sequence of SEQ ID NO: 516; and VL-CDR3 comprising the amino acid sequence of SEQ ID NO: 517; or
vv) CDR1 comprising the amino acid sequence of SEQ ID NO: 521; VH-CDR2 comprising the amino acid sequence of SEQ ID NO: 522; VH-CDR3 comprising the amino acid sequence of SEQ ID NO: 463; VL-CDR1 comprising the amino acid sequence of SEQ ID NO: 525; VL-CDR2 comprising the amino acid sequence of SEQ ID NO: 16; and VL-CDR3 comprising the amino acid sequence of SEQ ID NO: 467; or
ww) CDR1 comprising the amino acid sequence of SEQ ID NO: 371; VH-CDR2 comprising the amino acid sequence of SEQ ID NO: 532; VH-CDR3 comprising the amino acid sequence of SEQ ID NO: 533; VL-CDR1 comprising the amino acid sequence of SEQ ID NO: 345; VL-CDR2 comprising the amino acid sequence of SEQ ID NO: 376; and VL-CDR3 comprising the amino acid sequence of SEQ ID NO: 537; or
xx) CDR1 comprising the amino acid sequence of SEQ ID NO: 341; VH-CDR2 comprising the amino acid sequence of SEQ ID NO: 542; VH-CDR3 comprising the amino acid sequence of SEQ ID NO: 543; VL-CDR1 comprising the amino acid sequence of SEQ ID NO: 345; VL-CDR2 comprising the amino acid sequence of SEQ ID NO: 376; and VL-CDR3 comprising the amino acid sequence of SEQ ID NO: 347; or
yy) CDR1 comprising the amino acid sequence of SEQ ID NO: 551; VH-CDR2 comprising the amino acid sequence of SEQ ID NO: 552; VH-CDR3 comprising the amino acid sequence of SEQ ID NO: 553; VL-CDR1 comprising the amino acid sequence of SEQ ID NO: 555; VL-CDR2 comprising the amino acid sequence of SEQ ID NO: 16; and VL-CDR3 comprising the amino acid sequence of SEQ ID NO: 557; or
zz) CDR1 comprising the amino acid sequence of SEQ ID NO: 551; VH-CDR2 comprising the amino acid sequence of SEQ ID NO: 552; VH-CDR3 comprising the amino acid sequence of SEQ ID NO: 563; VL-CDR1 comprising the amino acid sequence of SEQ ID NO: 555; VL-CDR2 comprising the amino acid sequence of SEQ ID NO: 16; and VL-CDR3 comprising the amino acid sequence of SEQ ID NO: 557; or
aaa) CDR1 comprising the amino acid sequence of SEQ ID NO: 571; VH-CDR2 comprising the amino acid sequence of SEQ ID NO: 202; VH-CDR3 comprising the amino acid sequence of SEQ ID NO: 573; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107; or
bbb) CDR1 comprising the amino acid sequence of SEQ ID NO: 581; VH-CDR2 comprising the amino acid sequence of SEQ ID NO: 582; VH-CDR3 comprising the amino acid sequence of SEQ ID NO: 583; VL-CDR1 comprising the amino acid sequence of SEQ ID NO: 585; VL-CDR2 comprising the amino acid sequence of SEQ ID NO: 586; and VL-CDR3 comprising the amino acid sequence of SEQ ID NO: 587; or
ccc) CDR1 comprising the amino acid sequence of SEQ ID NO: 141; VH-CDR2 comprising the amino acid sequence of SEQ ID NO: 472; VH-CDR3 comprising the amino acid sequence of SEQ ID NO: 473; VL-CDR1 comprising the amino acid sequence of SEQ ID NO: 475; VL-CDR2 comprising the amino acid sequence of SEQ ID NO: 476; and VL-CDR3 comprising the amino acid sequence of SEQ ID NO: 477; or
ddd) CDR1 comprising the amino acid sequence of SEQ ID NO: 611; VH-CDR2 comprising the amino acid sequence of SEQ ID NO: 612; VH-CDR3 comprising the amino acid sequence of SEQ ID NO: 613; VL-CDR1 comprising the amino acid sequence of SEQ ID NO: 615; VL-CDR2 comprising the amino acid sequence of SEQ ID NO: 616; and VL-CDR3 comprising the amino acid sequence of SEQ ID NO: 617; or
eee) CDR1 comprising the amino acid sequence of SEQ ID NO: 621; VH-CDR2 comprising the amino acid sequence of SEQ ID NO: 622; VH-CDR3 comprising the amino acid sequence of SEQ ID NO: 623; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
fff) CDR1 comprising the amino acid sequence of SEQ ID NO: 631; VH-CDR2 comprising the amino acid sequence of SEQ ID NO: 632; VH-CDR3 comprising the amino acid sequence of SEQ ID NO: 633; VL-CDR1 comprising the amino acid sequence of SEQ ID NO: 635; VL-CDR2 comprising the amino acid sequence of SEQ ID NO: 616; and VL-CDR3 comprising the amino acid sequence of SEQ ID NO: 637; or
ggg) CDR1 comprising the amino acid sequence of SEQ ID NO: 641; VH-CDR2 comprising the amino acid sequence of SEQ ID NO: 642; VH-CDR3 comprising the amino acid sequence of SEQ ID NO: 643; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
hhh) CDR1 comprising the amino acid sequence of SEQ ID NO: 621; VH-CDR2 comprising the amino acid sequence of SEQ ID NO: 642; VH-CDR3 comprising the amino acid sequence of SEQ ID NO: 653; VL-CDR1 comprising the amino acid sequence of SEQ ID NO: 655; VL-CDR2 comprising the amino acid sequence of SEQ ID NO: 626; and VL-CDR3 comprising the amino acid sequence of SEQ ID NO: 627; or
iii) CDR1 comprising the amino acid sequence of SEQ ID NO: 661; VH-CDR2 comprising the amino acid sequence of SEQ ID NO: 662; VH-CDR3 comprising the amino acid sequence of SEQ ID NO: 663; VL-CDR1 comprising the amino acid sequence of SEQ ID NO: 665; VL-CDR2 comprising the amino acid sequence of SEQ ID NO: 666; and VL-CDR3 comprising the amino acid sequence of SEQ ID NO: 667; or
jjj) CDR1 comprising the amino acid sequence of SEQ ID NO: 671; VH-CDR2 comprising the amino acid sequence of SEQ ID NO: 672; VH-CDR3 comprising the amino acid sequence of SEQ ID NO: 673; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
kkk) CDR1 comprising the amino acid sequence of SEQ ID NO: 621; VH-CDR2 comprising the amino acid sequence of SEQ ID NO: 642; VH-CDR3 comprising the amino acid sequence of SEQ ID NO: 683; VL-CDR1 comprising the amino acid sequence of SEQ ID NO: 625; VL-CDR2 comprising the amino acid sequence of SEQ ID NO: 686; and VL-CDR3 comprising the amino acid sequence of SEQ ID NO: 627; or
lll) CDR1 comprising the amino acid sequence of SEQ ID NO: 691; VH-CDR2 comprising the amino acid sequence of SEQ ID NO: 692; VH-CDR3 comprising the amino acid sequence of SEQ ID NO: 693; VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697; or
mmm) CDR1 comprising the amino acid sequence of SEQ ID NO: 701; VH-CDR2 comprising the amino acid sequence of SEQ ID NO: 702; VH-CDR3 comprising the amino acid sequence of SEQ ID NO: 703; VL-CDR1 comprising the amino acid sequence of SEQ ID NO: 705; VL-CDR2 comprising the amino acid sequence of SEQ ID NO: 706; and VL-CDR3 comprising the amino acid sequence of SEQ ID NO: 707; or
nnn) CDR1 comprising the amino acid sequence of SEQ ID NO: 711; VH-CDR2 comprising the amino acid sequence of SEQ ID NO: 712; VH-CDR3 comprising the amino acid sequence of SEQ ID NO: 713; VL-CDR1 comprising the amino acid sequence of SEQ ID NO: 715; VL-CDR2 comprising the amino acid sequence of SEQ ID NO: 716; and VL-CDR3 comprising the amino acid sequence of SEQ ID NO: 717; or
ooo) CDR1 comprising the amino acid sequence of SEQ ID NO: 721; VH-CDR2 comprising the amino acid sequence of SEQ ID NO: 722; VH-CDR3 comprising the amino acid sequence of SEQ ID NO: 723; VL-CDR1 comprising the amino acid sequence of SEQ ID NO: 725; VL-CDR2 comprising the amino acid sequence of SEQ ID NO: 616; and VL-CDR3 comprising the amino acid sequence of SEQ ID NO: 637; or
ppp) CDR1 comprising the amino acid sequence of SEQ ID NO: 731; VH-CDR2 comprising the amino acid sequence of SEQ ID NO: 732; VH-CDR3 comprising the amino acid sequence of SEQ ID NO: 733; VL-CDR1 comprising the amino acid sequence of SEQ ID NO: 735; VL-CDR2 comprising the amino acid sequence of SEQ ID NO: 736; and VL-CDR3 comprising the amino acid sequence of SEQ ID NO: 637; or
qqq) CDR1 comprising the amino acid sequence of SEQ ID NO: 671; VH-CDR2 comprising the amino acid sequence of SEQ ID NO: 742; VH-CDR3 comprising the amino acid sequence of SEQ ID NO: 743; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
rrr) CDR1 comprising the amino acid sequence of SEQ ID NO: 751; VH-CDR2 comprising the amino acid sequence of SEQ ID NO: 722; VH-CDR3 comprising the amino acid sequence of SEQ ID NO: 723; VL-CDR1 comprising the amino acid sequence of SEQ ID NO: 735; VL-CDR2 comprising the amino acid sequence of SEQ ID NO: 616; and VL-CDR3 comprising the amino acid sequence of SEQ ID NO: 637; or
sss) CDR1 comprising the amino acid sequence of SEQ ID NO: 761; VH-CDR2 comprising the amino acid sequence of SEQ ID NO: 722; VH-CDR3 comprising the amino acid sequence of SEQ ID NO: 733; VL-CDR1 comprising the amino acid sequence of SEQ ID NO: 765; VL-CDR2 comprising the amino acid sequence of SEQ ID NO: 616; and VL-CDR3 comprising the amino acid sequence of SEQ ID NO: 637; or
ttt) CDR1 comprising the amino acid sequence of SEQ ID NO: 771; VH-CDR2 comprising the amino acid sequence of SEQ ID NO: 772; VH-CDR3 comprising the amino acid sequence of SEQ ID NO: 773; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
uuu) CDR1 comprising the amino acid sequence of SEQ ID NO: 751; VH-CDR2 comprising the amino acid sequence of SEQ ID NO: 722; VH-CDR3 comprising the amino acid sequence of SEQ ID NO: 723; VL-CDR1 comprising the amino acid sequence of SEQ ID NO: 735; VL-CDR2 comprising the amino acid sequence of SEQ ID NO: 616; and VL-CDR3 comprising the amino acid sequence of SEQ ID NO: 637; or
vvv) CDR1 comprising the amino acid sequence of SEQ ID NO: 791; VH-CDR2 comprising the amino acid sequence of SEQ ID NO: 792; VH-CDR3 comprising the amino acid sequence of SEQ ID NO: 793; VL-CDR1 comprising the amino acid sequence of SEQ ID NO: 795; VL-CDR2 comprising the amino acid sequence of SEQ ID NO: 616; and VL-CDR3 comprising the amino acid sequence of SEQ ID NO: 797; or
www) CDR1 comprising the amino acid sequence of SEQ ID NO: 771; VH-CDR2 comprising the amino acid sequence of SEQ ID NO: 802; VH-CDR3 comprising the amino acid sequence of SEQ ID NO: 803; VL-CDR1 comprising the amino acid sequence of SEQ ID NO: 805; VL-CDR2 comprising the amino acid sequence of SEQ ID NO: 676; and VL-CDR3 comprising the amino acid sequence of SEQ ID NO: 677; or
xxx) CDR1 comprising the amino acid sequence of SEQ ID NO: 811; VH-CDR2 comprising the amino acid sequence of SEQ ID NO: 812; VH-CDR3 comprising the amino acid sequence of SEQ ID NO: 813; VL-CDR1 comprising the amino acid sequence of SEQ ID NO: 815; VL-CDR2 comprising the amino acid sequence of SEQ ID NO: 696; and VL-CDR3 comprising the amino acid sequence of SEQ ID NO: 817; or
yyy) CDR1 comprising the amino acid sequence of SEQ ID NO: 821; VH-CDR2 comprising the amino acid sequence of SEQ ID NO: 822; VH-CDR3 comprising the amino acid sequence of SEQ ID NO: 823; VL-CDR1 comprising the amino acid sequence of SEQ ID NO: 825; VL-CDR2 comprising the amino acid sequence of SEQ ID NO: 826; and VL-CDR3 comprising the amino acid sequence of SEQ ID NO: 827; or
zzz) CDR1 comprising the amino acid sequence of SEQ ID NO: 831; VH-CDR2 comprising the amino acid sequence of SEQ ID NO: 832; VH-CDR3 comprising the amino acid sequence of SEQ ID NO: 833; VL-CDR1 comprising the amino acid sequence of SEQ ID NO: 835; VL-CDR2 comprising the amino acid sequence of SEQ ID NO: 836; and VL-CDR3 comprising the amino acid sequence of SEQ ID NO: 817; or
aaaa) CDR1 comprising the amino acid sequence of SEQ ID NO: 841; VH-CDR2 comprising the amino acid sequence of SEQ ID NO: 842; VH-CDR3 comprising the amino acid sequence of SEQ ID NO: 843; VL-CDR1 comprising the amino acid sequence of SEQ ID NO: 845; VL-CDR2 comprising the amino acid sequence of SEQ ID NO: 846; and VL-CDR3 comprising the amino acid sequence of SEQ ID NO: 847.

In some embodiments, the biparatopic antibody or a functional fragment thereof comprises a first binding site and a second distinct binding site selected from the group consisting of binding sites comprising:
a) VH-CDR1 comprising the amino acid sequence of SEQ ID NO: 11; VH-CDR2 comprising the amino acid sequence of SEQ ID NO: 12; VH-CDR3 comprising the amino acid sequence of SEQ ID NO: 13; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 17; or
b) VH-CDR1 comprising the amino acid sequence of SEQ ID NO: 31; VH-CDR2 comprising the amino acid sequence of SEQ ID NO: 32; VH-CDR3 comprising the amino acid sequence of SEQ ID NO: 33; VL-CDR1 comprising the amino acid sequence of SEQ ID NO: 85; VL-CDR2 comprising the amino acid sequence of SEQ ID NO: 36; and VL-CDR3 comprising the amino acid sequence of SEQ ID NO: 87; or
c) VH-CDR1 comprising the amino acid sequence of SEQ ID NO: 21; VH-CDR2 comprising the amino acid sequence of SEQ ID NO: 52; VH-CDR3 comprising the amino acid sequence YSF; VL-CDR1 comprising the amino acid sequence of SEQ ID NO: 55; VL-CDR2 comprising the amino acid sequence of SEQ ID NO: 56; and VL-CDR3 comprising the amino acid sequence of SEQ ID NO: 27; or
d) VH-CDR1 comprising the amino acid sequence of SEQ ID NO: 91; VH-CDR2 comprising the amino acid sequence of SEQ ID NO: 92; and VH-CDR3 comprising the amino acid sequence of SEQ ID NO: 93; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 97; or
e) VH-CDR1 comprising the amino acid sequence of SEQ ID NO: 151; VH-CDR2 comprising the amino acid sequence of SEQ ID NO: 152; VH-CDR3 comprising the amino acid sequence of SEQ ID NO: 153; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107; or
f) VH-CDR1 comprising the amino acid sequence of SEQ ID NO: 181; VH-CDR2 comprising the amino acid sequence of SEQ ID NO: 182; VH-CDR3 comprising the amino acid sequence of SEQ ID NO: 183; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 187; or
g) VH-CDR1 comprising the amino acid sequence of SEQ ID NO: 321; VH-CDR2 comprising the amino acid sequence of SEQ ID NO: 322; VH-CDR3 comprising the amino acid sequence of SEQ ID NO: 323; VL-CDR1 comprising the amino acid sequence of SEQ ID NO: 325; VL-CDR2 comprising the amino acid sequence of SEQ ID NO: 326; and VL-CDR3 comprising the amino acid sequence of SEQ ID NO: 327; or
h) VH-CDR1 comprising the amino acid sequence of SEQ ID NO: 151; VH-CDR2 comprising the amino acid sequence of SEQ ID NO: 332; VH-CDR3 comprising the amino acid sequence of SEQ ID NO: 333; VL-CDR1 comprising the amino acid sequence of SEQ ID NO: 335; VL-CDR2 comprising the amino acid sequence of SEQ ID NO: 336; and VL-CDR3 comprising the amino acid sequence of SEQ ID NO: 107; or
i) VH-CDR1 comprising the amino acid sequence of SEQ ID NO: 341; VH-CDR2 comprising the amino acid sequence of SEQ ID NO: 342; VH-CDR3 comprising the amino acid sequence of SEQ ID NO: 343; VL-CDR1 comprising the amino acid sequence of SEQ ID NO: 345; VL-CDR2 comprising the amino acid sequence of SEQ ID NO: 346; and VL-CDR3 comprising the amino acid sequence of SEQ ID NO: 347; or
j) VH-CDR1 comprising the amino acid sequence of SEQ ID NO: 361; VH-CDR2 comprising the amino acid sequence of SEQ ID NO: 362; VH-CDR3 comprising the amino acid sequence of SEQ ID NO: 363; VL-CDR1 comprising the amino acid sequence of SEQ ID NO: 365; VL-CDR2 comprising the amino acid sequence of SEQ ID NO: 16; and VL-CDR3 comprising the amino acid sequence of SEQ ID NO: 367; or
k) VH-CDR1 comprising the amino acid sequence of SEQ ID NO: 351; VH-CDR2 comprising the amino acid sequence of SEQ ID NO: 382; VH-CDR3 comprising the amino acid sequence of SEQ ID NO: 393; VL-CDR1 comprising the amino acid sequence of SEQ ID NO: 405; VL-CDR2 comprising the amino acid sequence of SEQ ID NO: 356; and VL-CDR3 comprising the amino acid sequence of SEQ ID NO: 357; or
l) VH-CDR1 comprising the amino acid sequence of SEQ ID NO: 461; VH-CDR2 comprising the amino acid sequence of SEQ ID NO: 462; VH-CDR3 comprising the amino acid sequence of SEQ ID NO: 463; VL-CDR1 comprising the amino acid sequence of SEQ ID NO: 465; VL-CDR2 comprising the amino acid sequence of SEQ ID NO: 16; and VL-CDR3 comprising the amino acid sequence of SEQ ID NO: 467; or
m) VH-CDR1 comprising the amino acid sequence of SEQ ID NO: 311; VH-CDR2 comprising the amino acid sequence of SEQ ID NO: 512; VH-CDR3 comprising the amino acid sequence of SEQ ID NO: 513; VL-CDR1 comprising the amino acid sequence of SEQ ID NO: 515; VL-CDR2 comprising the amino acid sequence of SEQ ID NO: 516; and VL-CDR3 comprising the amino acid sequence of SEQ ID NO: 517; or
n) VH-CDR1 comprising the amino acid sequence of SEQ ID NO: 371; VH-CDR2 comprising the amino acid sequence of SEQ ID NO: 532; VH-CDR3 comprising the amino acid sequence of SEQ ID NO: 533; VL-CDR1 comprising the amino acid sequence of SEQ ID NO: 345; VL-CDR2 comprising the amino acid sequence of SEQ ID NO: 376; and VL-CDR3 comprising the amino acid sequence of SEQ ID NO: 537; or
o) a VH-CDR1 comprising the amino acid sequence of SEQ ID NO: 571; VH-CDR2 comprising the amino acid sequence of SEQ ID NO: 202; VH-CDR3 comprising the amino acid sequence of SEQ ID NO: 573; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107; or
p) VH-CDR1 comprising the amino acid sequence of SEQ ID NO: 141; VH-CDR2 comprising the amino acid sequence of SEQ ID NO: 472; VH-CDR3 comprising the amino acid sequence of SEQ ID NO: 473; VL-CDR1 comprising the amino acid sequence of SEQ ID NO: 475; VL-CDR2 comprising the amino acid sequence of SEQ ID NO: 476; and VL-CDR3 comprising the amino acid sequence of SEQ ID NO: 477; or
q) VH-CDR1 comprising the amino acid sequence of SEQ ID NO: 611; VH-CDR2 comprising the amino acid sequence of SEQ ID NO: 612; VH-CDR3 comprising the amino acid sequence of SEQ ID NO: 613; VL-CDR1 comprising the amino acid sequence of SEQ ID NO: 615; VL-CDR2 comprising the amino acid sequence of SEQ ID NO: 616; and VL-CDR3 comprising the amino acid sequence of SEQ ID NO: 617; or
r) VH-CDR1 comprising the amino acid sequence of SEQ ID NO: 671; VH-CDR2 comprising the amino acid sequence of SEQ ID NO: 672; VH-CDR3 comprising the amino acid sequence of SEQ ID NO: 673; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
s) VH-CDR1 comprising the amino acid sequence of SEQ ID NO: 691; VH-CDR2 comprising the amino acid sequence of SEQ ID NO: 692; VH-CDR3 comprising the amino acid sequence of SEQ ID NO: 693; VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697.

In some embodiments, the biparatopic antibody or a functional fragment thereof comprises:
a) a first binding site comprising a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second binding site comprising a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; or
b) a first binding site comprising a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second binding site comprising a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; or
c) a first binding site comprising a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second binding site comprising a VH-CDR1 comprising the amino acid sequence of SEQ ID NO: 91; a VH-CDR2 comprising the amino acid sequence of SEQ ID NO: 92; and a VH-CDR3 comprising the amino acid sequence of SEQ ID NO: 93; VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
d) a first binding site comprising a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second binding site comprising a VH-CDR1 comprising the amino acid sequence of SEQ ID NO: 31; VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; or
e) a first binding site comprising a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second binding site comprising a VH-CDR1 comprising the amino acid sequence of SEQ ID NO: 91; a VH-CDR2 comprising the amino acid sequence of SEQ ID NO: 92; and a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
f) a first binding site comprising a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second binding site comprising a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
g) a first binding site comprising a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second binding site comprising a VH-CDR1 comprising the amino acid sequence of SEQ ID NO: 181; a VH-CDR2 comprising the amino acid sequence of SEQ ID NO: 182; and a VH-CDR3 comprising the amino acid sequence of SEQ ID NO: 183; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 187; or
h) a first binding site comprising a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; and a second binding site comprising a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a first binding site comprising a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; and a second binding site comprising a VH-CDR1 comprising the amino acid sequence of SEQ ID NO: 181; a VH-CDR2 comprising the amino acid sequence of SEQ ID NO: 182; and a VH-CDR3 comprising the amino acid sequence of SEQ ID NO: 183; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 187; or
j) a first pair of variable regions VH and VL comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
k) a first pair of variable regions VH and VL comprises a VH-CDR1 comprising the amino acid sequence of SEQ ID NO: 691; a VH-CDR2 comprising the amino acid sequence of SEQ ID NO: 692; a VH-CDR3 comprising the amino acid sequence of SEQ ID NO: 693; a VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; and a second pair of variable regions VH and VL comprises a VH-CDR1 comprising the amino acid sequence of SEQ ID NO: 671; a VH-CDR2 comprising the amino acid sequence of SEQ ID NO: 672; and a VH-CDR3 comprising the amino acid sequence of SEQ ID NO: 673; VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; or
l) a first pair of variable regions VH and VL comprises a VH-CDR1 comprising the amino acid sequence of SEQ ID NO: 321; a VH-CDR2 comprising the amino acid sequence of SEQ ID NO: 322; a VH-CDR3 comprising the amino acid sequence of SEQ ID NO: 323; a VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
m) a first pair of variable regions VH and VL comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
n) a first pair of variable regions VH and VL comprises a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second pair of variable regions VH and VL comprises a VH-CDR1 comprising the amino acid sequence of SEQ ID NO: 361; a VH-CDR2 comprising the amino acid sequence of SEQ ID NO: 362; and a VH-CDR3 comprising the amino acid sequence of SEQ ID NO: 363; VL-CDR1 comprising the amino acid sequence of SEQ ID NO: 365; a VL-CDR2 comprising the amino acid sequence of SEQ ID NO: 16; and a VL-CDR3 comprising the amino acid sequence of SEQ ID NO: 367; or
o) a first pair of variable regions VH and VL comprises a VH-CDR1 comprising the amino acid sequence of SEQ ID NO: 351; a VH-CDR2 comprising the amino acid sequence of SEQ ID NO: 382; a VH-CDR3 comprising the amino acid sequence of SEQ ID NO: 393; a VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; and a second pair of variable regions VH and VL comprises a VH-CDR1 comprising the amino acid sequence of SEQ ID NO: 91; a VH-CDR2 comprising the amino acid sequence of SEQ ID NO: 92; and a VH-CDR3 comprising the amino acid sequence of SEQ ID NO: 93; VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
p) a first pair of variable regions VH and VL comprises a VH-CDR1 comprising the amino acid sequence of SEQ ID NO: 371; a VH-CDR2 comprising the amino acid sequence of SEQ ID NO: 532; a VH-CDR3 comprising the amino acid sequence of SEQ ID NO: 533; a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
q) a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 371; a VH-CDR2 comprising the amino acid sequence of SEQ ID NO: 532; and a VH-CDR3 comprising the amino acid sequence of SEQ ID NO: 533; VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; or
r) a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
s) a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
t) a first pair of variable regions VH and VL comprises a VH-CDR1 comprising the amino acid sequence of SEQ ID NO: 371; a VH-CDR2 comprising the amino acid sequence of SEQ ID NO: 532; a VH-CDR3 comprising the amino acid sequence of SEQ ID NO: 533; a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
u) a first pair of variable regions VH and VL comprises a VH-CDR1 comprising the amino acid sequence of SEQ ID NO: 321; a VH-CDR2 comprising the amino acid sequence of SEQ ID NO: 322; a VH-CDR3 comprising the amino acid sequence of SEQ ID NO: 323; a VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
v) a first pair of variable regions VH and VL comprises a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
w) a first pair of variable regions VH and VL comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
x) a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
y) a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
z) a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
aa) a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 321; a VH-CDR2 comprising the amino acid sequence of SEQ ID NO: 322; and a VH-CDR3 comprising the amino acid sequence of SEQ ID NO: 323; VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; or
bb) a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 571; a VH-CDR2 comprising the amino acid sequence of SEQ ID NO: 202; and a VH-CDR3 comprising the amino acid sequence of SEQ ID NO: 573; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
cc) a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 341; a VH-CDR2 comprising the amino acid sequence of SEQ ID NO: 342; and a VH-CDR3 comprising the amino acid sequence of SEQ ID NO: 343; VL-CDR1 comprising the amino acid sequence of SEQ ID NO: 345; a VL-CDR2 comprising the amino acid sequence of SEQ ID NO: 346; and a VL-CDR3 comprising the amino acid sequence of SEQ ID NO: 347; or
dd) a first pair of variable regions VH and VL comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; and a second pair of variable regions VH and VL comprises a VH-CDR1 comprising the amino acid sequence of SEQ ID NO: 141; a VH-CDR2 comprising the amino acid sequence of SEQ ID NO: 472; and a VH-CDR3 comprising the amino acid sequence of SEQ ID NO: 473; VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; or
ee) a first pair of variable regions VH and VL comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; and a second pair of variable regions VH and VL comprises a VH-CDR1 comprising the amino acid sequence of SEQ ID NO: 311; a VH-CDR2 comprising the amino acid sequence of SEQ ID NO: 512; and a VH-CDR3 comprising the amino acid sequence of SEQ ID NO: 513; VL-CDR1 comprising the amino acid sequence of SEQ ID NO: 515; a VL-CDR2 comprising the amino acid sequence of SEQ ID NO: 516; and a VL-CDR3 comprising the amino acid sequence of SEQ ID NO: 517; or
ff) a first pair of variable regions VH and VL comprises a VH-CDR1 comprising the amino acid sequence of SEQ ID NO: 341; a VH-CDR2 comprising the amino acid sequence of SEQ ID NO: 342; a VH-CDR3 comprising the amino acid sequence of SEQ ID NO: 343; a VL-CDR1 comprising the amino acid sequence of SEQ ID NO: 345; a VL-CDR2 comprising the amino acid sequence of SEQ ID NO: 346; and a VL-CDR3 comprising the amino acid sequence of SEQ ID NO: 347; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
gg) a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
hh) a first pair of variable regions VH and VL comprises a VH-CDR1 comprising the amino acid sequence of SEQ ID NO: 691; a VH-CDR2 comprising the amino acid sequence of SEQ ID NO: 692; a VH-CDR3 comprising the amino acid sequence of SEQ ID NO: 693; a VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
ii) a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 151; a VH-CDR2 comprising the amino acid sequence of SEQ ID NO: 332; and a VH-CDR3 comprising the amino acid sequence of SEQ ID NO: 333; VL-CDR1 comprising the amino acid sequence of SEQ ID NO: 335; a VL-CDR2 comprising the amino acid sequence of SEQ ID NO: 336; and a VL-CDR3 comprising the amino acid sequence of SEQ ID NO: 107; or
jj) a first pair of variable regions VH and VL comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
kk) a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 611; a VH-CDR2 comprising the amino acid sequence of SEQ ID NO: 612; and a VH-CDR3 comprising the amino acid sequence of SEQ ID NO: 613; VL-CDR1 comprising the amino acid sequence of SEQ ID NO: 615; a VL-CDR2 comprising the amino acid sequence of SEQ ID NO: 616; and a VL-CDR3 comprising the amino acid sequence of SEQ ID NO: 617.

In some embodiments, the biparatopic antibody or a functional fragment thereof comprises:
a. a first pair of variable regions VH and VL comprises a VH-CDR1 comprising the amino acid sequence of SEQ ID NO: 321; a VH-CDR2 comprising the amino acid sequence of SEQ ID NO: 322; a VH-CDR3 comprising the amino acid sequence of SEQ ID NO: 323; a VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
b. a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
c. a first pair of variable regions VH and VL comprises a VH-CDR1 comprising the amino acid sequence of SEQ ID NO: 371; a VH-CDR2 comprising the amino acid sequence of SEQ ID NO: 532; a VH-CDR3 comprising the amino acid sequence of SEQ ID NO: 533; a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
d. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
e. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
f. a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 321; a VH-CDR2 comprising the amino acid sequence of SEQ ID NO: 322; and a VH-CDR3 comprising the amino acid sequence of SEQ ID NO: 323; VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; or
g. a first pair of variable regions VH and VL comprises a VH-CDR1 comprising the amino acid sequence of SEQ ID NO: 691; a VH-CDR2 comprising the amino acid sequence of SEQ ID NO: 692; a VH-CDR3 comprising the amino acid sequence of SEQ ID NO: 693; a VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
h. a first pair of variable regions VH and VL comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17.

In a further embodiment, the biparatopic antibody or a functional fragment thereof comprises:
a. a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
b. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107.

In some embodiments, a method of preventing, alleviating and/or treating a disease, disorder or abnormality associated with alpha-synuclein aggregates or pathological alpha-synuclein, such as from Parkinson's disease (sporadic, familial with alpha-synuclein mutations, familial with mutations other than alpha-synuclein, pure autonomic failure and Lewy body dysphagia), Parkinson's disease with dementia, Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder, is provided. According to one embodiment, the methods of the invention comprise administering an effective concentration or an effective amount of a biparatopic antigen-binding molecule, particularly a biparatopic antibody, or a functional fragment thereof, or a mixture comprising at least two monospecific antibodies or functional fragments thereof, or mixtures of biparatopic antibodies or functional fragments thereof and at least one monospecific monoclonal antibody or a functional fragment thereof, or a composition of the invention, as described herein to a subject in need thereof.

In another embodiment, a biparatopic antibody or a functional fragment thereof, or a mixture of two monospecific antibodies or functional fragments thereof as described herein is administered to prevent, alleviate or treat a disease, disorder or abnormality associated with alpha-synuclein aggregates selected from Parkinson's Disease, Multiple System Atrophy, Lewy Body dementia (LBD; including dementia with Lewy bodies (DLB) (“pure” Lewy body dementia), Parkinson's disease dementia (PDD)), or Diffuse Lewy Body Disease.

In some embodiments, an isolated biparatopic antibody, or a functional fragment thereof, described herein is provided for use as a medicament. In some embodiments, an isolated biparatopic antibody, or a functional fragment thereof, described herein is provided for use in alleviating, preventing and/or treating a a CNS disease, in particular a synucleinopathy in a subject. In some embodiments, use of a biparatopic antibody, or a functional fragment thereof, described herein is provided for manufacture of a medicament for preventing, alleviating and/or treating a disease, a disorder and/or abnormality associated with alpha-synuclein aggregates.

An “antigen binding molecule” as used herein, is any molecule that can specifically or selectively bind to an antigen or epitope. A binding molecule may include or be an antibody, a fragment or derivatives thereof. An alpha-synuclein binding molecule is a molecule that binds to the alpha-synuclein protein or alpha-synuclein peptide at a specific recognition site, epitope, such as an alpha-synuclein antibody or fragment thereof.

A “biparatopic antigen-binding molecule,” as used herein, is a molecule that can specifically or selectively bind to at least two distinct antigens/epitopes simultaneously. A biparatopic binding molecule may include or be a biparatopic antibody or a functional fragment or derivative thereof (e.g. scFv, Fabs Fab′ fragment, F(ab′)2 fragment or VHH). An alpha-synuclein biparatopic binding molecule is a molecule that binds at least two recognition sites, epitopes of an alpha-synuclein protein. A biparatopic binding molecule may include or be a biparatopic antibody or a functional fragment thereof. The biparatopic antigen-binding molecule or functional fragment thereof of the invention can be further modified to a multispecific antibody by introducing binding site or polypeptide fragment able to modulate Fc mediated function and/or FcRn binding and/or blood brain barrier penetration. The biparatopic antigen-binding molecule of the invention can also be delivered as a corresponding nucleic acid encoding for the biparatopic antigen-binding molecule.

Such nucleic acid molecule may be a part of a viral vector for targeted delivery to the blood brain barrier or any other cell type in the CNS. A viral vector may be a recombinant adeno-associated viral vectors (rAAV) selected from any AAV serotype known in the art, including, without limitation, from AAV1 to AAV12 to enable the biparatopic antigen-binding molecule to be expressed intracellularly or into the brain parenchyma.

The term “distinct epitope” or “distinct antigens” refers to epitopes which differs by at least one amino acid residues. In some embodiment of the invention, distinct epitopes have in common at least one, in particular at least two, more particularly at least 3, even more particularly at least 4 amino acid residues. In some embodiment of the invention, distinct epitopes have no amino acid residues in common.

Accordingly, in context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments or derivatives thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv), VHH or antibody-fusion proteins.

Humanized antibodies are modified antibodies that are also referred to as reshaped human antibodies. A humanized antibody is constructed by transferring the CDRs of an antibody derived from an immunized animal onto the accepting framework of a human germline antibody. Conventional genetic recombination techniques for such purposes are known (see European Patent Application Publication No. EP 239400; International Publication No. WO 96/02576; Sato K. et al., Cancer Research 1993, 53: 851-856; International Publication No. WO 99/51743).

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. VH-CDR, or CDR-H depicts a CDR region of a heavy chain and VL-CDR or CDR-L relates to a CDR region of a light chain. VH means the variable domain of the heavy chain and VL means the variable domain of the light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J., Mol. Biol. 196 (1987), 901-917 or Chothia, Nature 342 (1989), 877-883. The CDRs provided herein are determined according to Kabat. However, the CDRs of the antibodies of the invention and fragments thereof may be defined according to any known numbering system, as would be readily understood by the skilled person. Thus, according to all aspects, the antibodies of the invention and fragments thereof may comprise 1, 2 and preferably all 3 CDRs from any of the specified VH and VL sequences herein.

An “Fc” region contains two heavy chain fragments each comprising the CH2 and CH3 domains of an antibody. The two heavy chain fragments are held together by two or more disulfide bonds and by interactions of the CH3 domains.

A “Fab fragment” contains one light chain and a portion of one heavy chain that contains the VH domain and the CH1 domain containing a cysteine residue to form the disulfide bridge between the two polypeptidique chain. Fab may refer to this region in isolation, or this region in the context of a full length antibody or antibody fragment.

A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains.

The “Fv region” comprises the variable regions from both the heavy and light chains, but lacks the constant regions.

Accordingly, in the context of this invention, biparatopic antigen-binding molecules, such as biparatopic antibodies or functional fragments thereof, and mixtures comprising at least two monospecific antibodies or functional fragments thereof, a are provided, which are murine, chimeric or humanized and can successfully be employed in compositions.

An “antibody that binds to an epitope” within a defined region of a protein is an antibody that requires the presence of one or more of the amino acids within that region for binding to the protein.

In certain embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody is determined by FACS, WB or by a suitable binding assay such as ELISA.

Accordingly, specificity can be determined experimentally by methods known in the art and methods as described herein. Such methods comprise, but are not limited to Western Blots, ELISA-, RIA-, ECL-, IRMA-tests and peptide scans.

It may be understood by a person skilled in the art that the epitopes may be comprised in the alpha-synuclein protein, but may also be comprised in a degradation product thereof or may be a chemically synthesized peptide. The amino acid positions are only indicated to demonstrate the position of the corresponding amino acid sequence in the sequence of the alpha-synuclein protein. The invention encompasses all peptides comprising the epitope. The peptide may be a part of a polypeptide of more than 100 amino acids in length or may be a small peptide of less than 100, particularly less than 50, more particularly less than 25 amino acids, even more particularly less than 18 amino adds. The amino acids of such peptide may be natural amino acids or nonnatural amino acids (e.g., beta-amino adds, gamma-amino adds, D-amino adds) or a combination thereof. Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty add, a sugar moiety or may be inserted in a membrane. In order to test whether an antibody in question and the antibody of the present invention recognize the same or similar epitope, many assays are known in the art, some of which (e.g. “alanine scanning mutagenesis”) are described in the examples below.

In accordance with the above, in certain embodiments, amino add sequence variants of the biparatopic antibodies or functional fragments thereof provided herein are contemplated. For example, it may be desirable to improve the binding affinity and/or other biological properties of the biparatopic antibody or a functional fragment thereof. Amino add sequence variants of an antibody or a functional fragment thereof may be prepared by introducing appropriate modifications into the nucleotide sequence encoding the antibody or a functional fragment thereof, or by peptide synthesis. Such modifications include, for example, deletions, and/or insertions and/or substitutions of residues within the amino acid sequence of the antibody or a functional fragment thereof. Any combination of deletion, insertion, and substitution can be made to arrive at the final construct, provided that the final construct possesses the desired characteristics, e.g., antigen-binding.

In certain embodiments, biparatopic antibody variants or functional fragment variants having one or more amino acid substitutions are provided. Sites of interest for substitutional mutagenesis include the CDRs, FRs and Fc region. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into a biparatopic antibody of interest or antibodies of the composition and the products screened for a desired activity, e.g., retained/improved antigen binding, decreased immunogenicity, or improved ADCC or CDC.

TABLE 1

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

In certain embodiments, one or more amino acid modifications may be introduced into the Fc region of a biparatopic antibody or active fragments thereof, or monospecific antibodies of the mixture provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the Fc region is mutated to increase its affinity to FcRn at pH6.0 and consequently extend the antibody half-life. Antibodies with enhanced affinity to FcRn include those with substitution of one or more of Fc region residues 252, 253, 254, 256, 428, 434, including the so called YTE mutation with substitution M252Y/S254T/T256E (Dall' Acqua et al, J Immunol. 169:5171-5180 (2002)) or LS mutation M428L/N434S (Zalevsky et al, Nat Biotechnol. 28(2): 157-159 (2010)).

In certain embodiments, it may be desirable to create cysteine engineered antibodies, e.g., “thioMAbs,” in which one or more residues of an antibody are substituted with cysteine residues.

In particular embodiments, the substituted residues occur at accessible sites of the antibody. The accessible sites may be on the antibody surface. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541 and in Bhakta S., Raab H., Junutula J. R. (2013) Engineering THIOMABs for Site-Specific Conjugation of Thiol-Reactive Linkers. In: Ducry L. (eds) Antibody-Drug Conjugates. Methods in Molecular Biology (Methods and Protocols), vol 1045. Humana Press, Totowa, NJ. https://doi.org/10.1007/978-1-62703-541-5_11.

In certain embodiments, an antibody provided herein may be further modified to contain additional nonproteinaceous moieties. Suitable nonproteinaceous moieties are known in the art and readily available. Moieties suitable for derivatization of the antibody include but are not limited to water soluble polymers. Nonlimiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n¬ vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, the invention contemplates a biparatopic antibody variant or active fragments thereof, or a mixture comprising at least two alpha-synuclein monospecific antibody variants, that possesses some but not all effector functions, which make it a desirable candidate for applications in which the half life of the antibody in vivo is important yet certain effector functions (such as complement activation and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes and microglia express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)).

Antibodies with reduced effector function include those with substitution of one or more of Fc region residues 234, 235, 238, 265, 269, 270, 297, 327 and 329 (U.S. Pat. No. 6,737,056). Certain antibody variants with improved or diminished binding to Fc gamma receptors (FcgRs) are described. (See, e.g., U.S. Pat. No. 6,737,056; WO 2004/056312, and Shields et al., J. Biol. Chem. 9(2): 6591-6604 (2001)). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581) or the so-called “DANG” Fc mutant with substitution of residues 265 to alanine and 297 to glycine. Alternatively, antibodies with reduced effector function include those with substitution of one or more of Fc region residues 234, 235 and 329, so-called “LALA-PG” Fc mutant with substitution of residues 234 and 235 to alanine and 329 to glycine (Lo, M. et al., Journal of Biochemistry, 292, 3900-3908 (2017)). Antibodies from the human IgG4 isotype include mutations S228P/L235E to stabilize the hinge and to reduce FcR binding (Schlothauer et al, PEDS, 29 (10):457-466).

Other Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821.

Biparatopic antigen-binding molecules of the invention can be produced by a variety of methods including, but not limited to, fusion of hybridomas or linking of Fab′ fragment (Songsivilai & Lachmann, Clin. Exp. Immunol. 79:315-321 (1990); Kostelny et al., J. Immunol. 148, 1547-1553 (1992); Ulrich Brinkmann & Roland E. Kontermann (2017) The making of bispecific antibodies, mAbs, 9:2, 182-212).

Any suitable technology may be used in the production of the biparatopic antigen-binding molecules of the invention. Various technologies exist in order to improve the efficiency of bispecific antibody production. Several approaches have modified the natural constant (including CH1-CL) domains to enable the correct formation of the bispecific antibody arms. Schaefer et al., PNAS, 2011, 108 (27) 11187-11192 described the exchange of CH1 and CL domain to correctly assemble heavy and light chains. The natural TCR α/β heterodimers were also used to replace the CH1/CL domains and produce IgG-like molecules (Wu et al., Mabs, 2015, 7(2), 364-376 and WO2019/057122). Others also used artificially introduced disulfide bond in the heavy and light chain constant domain to enhance cognate chain assembly (Mazor et al, mAbs, 2015, 7(2): 377-389). Labrjn et al, PNAS, 2013 110 (13) 5145-5150 describe a process that involves separate expression of two parental antibodies, each containing single matched point mutations in the CH3 domains. The parental antibodies are mixed and subjected to controlled reducing conditions in vitro that separate the antibodies into HL half-molecules and allow reassembly and reoxidation to form highly pure bsAbs. Thus, antibodies of the invention can incorporate any of the relevant constant domain modifications that characterise bispecific antibody production methods. For example an antibody includes a molecule in which the CH1 and CL domains are replaced with TCR α/β heterodimers. Constant domain sequences given herein are according to the EU numbering scheme. As the skilled person would be aware, any suitable numbering scheme may be adopted.

It should be noted that where pairs of VH/VL sequences (or arms) are specified herein comprised within a biparatopic antibody or functional fragment thereof, this does not imply the order of the sequences relative to one another unless indicated otherwise. A number of bispecific production technologies can produce assymetric architecture and, unless specified otherwise, both forms of the antibodies or functional fragments thereof are intended to be encompassed. For example, if VH/VL A (Arm A) and VH/VL B (Arm B) form a bispecific antibody in the context of a knob-into-hole structure, Arm A can form the chain comprising the Fc knob and the Arm B can form the chain comprising the Fc hole, or vice versa.

The invention further provides methods of manufacturing a biparatopic antibody binding to a protein associated with a CNS disease, such as alpha-synuclein, Tau, TDP-43, ASC, NLRP3, C5a, C1q, C3, huntingtin or prion protein, in particular an alpha-synuclein biparatopic antibody or a functional fragment thereof, such methods comprises the steps of:
Culturing host cells comprising at least one nucleic acid molecule capable of encoding the biparatopic antibody or a functional fragment thereof (hereinafter referred as “nucleic acid of the invention”) under conditions that allow expression of the biparatopic binding molecule of the invention binding to a protein associated with a CNS disease, such as alpha-synuclein, Tau, TDP-43, ASC, NLRP3, C5a, C1q, C3, huntingtin or prion protein, in particular the alpha-synuclein biparatopic binding molecule of the invention; and,
Recovering, purifying or isolating the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof expressed by the host cells from the culture; and
Optionally further modifying and/or formulating the biparatopic antibody or a functional fragment thereof.

The invention further provides methods of manufacturing a biparatopic antibody binding to a protein associated with a CNS disease, such as alpha-synuclein, Tau, TDP-43, ASC, NLRP3, C5a, C1q, C3, huntingtin or prion protein, in particular an alpha-synuclein biparatopic antibody or a functional fragment thereof, such methods comprises the steps of:
Culturing a cell-free expression system comprising at least one nucleic acid molecule capable of encoding the biparatopic antibody or a functional fragment thereof (hereinafter referred as “nucleic acid of the invention”) under conditions that allow expression of the biparatopic binding molecule of the invention binding to a protein associated with a CNS disease, such as alpha-synuclein, Tau, TDP-43, ASC, NLRP3, C5a, C1q, C3, huntingtin or prion protein, in particular the alpha-synuclein biparatopic binding molecule of the invention; and,
Recovering, purifying or isolating the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof from the culture; and
Optionally further modifying and/or formulating the biparatopic antibody or a functional fragment thereof.

The invention further provides methods of manufacturing a biparatopic antibody or a functional fragment thereof, such methods comprises the steps of:
Culturing host cells comprising at least one nucleic acid molecule capable of encoding a first binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof (hereinafter referred as “nucleic acid of the invention”) under conditions that allow expression of the first binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof; and,
Culturing host cells comprising at least one nucleic acid molecule capable of encoding a second binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof (hereinafter referred as “nucleic acid of the invention”) under conditions that allow expression of the second binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof; and,
Recovering, purifying or isolating each of the binding sites of the biparatopic antibody or a functional fragment thereof expressed by the respective host cells from the respective culture; and,
In vitro combining the two binding sites of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof to form the biparatopic biparatopic antibody or a functional fragment thereof;
Optionally further purifying, modifying and/or formulating the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof.

The invention further provides methods of manufacturing a biparatopic antibody or a functional fragment thereof, such methods comprises the steps of:
Culturing a cell-free expression system comprising at least one nucleic acid molecule capable of encoding a first binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof (hereinafter referred as “nucleic acid of the invention”) under conditions that allow expression of the first binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof; and,
Culturing a cell-free expression system comprising at least one nucleic acid molecule capable of encoding a second binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof (hereinafter referred as “nucleic acid of the invention”) under conditions that allow expression of the second binding site of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof; and,
Recovering, purifying or isolating each of the binding sites of the biparatopic antibody or a functional fragment thereof expressed by the respective host cells from the respective culture; and,
In vitro combining the two binding sites of the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof to form the biparatopic biparatopic antibody or a functional fragment thereof;
Optionally further purifying, modifying and/or formulating the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof.

In some embodiments, an isolated nucleic acid is provided, wherein the isolated nucleic acid encodes a biparatopic antibody described herein.

The invention further provides a cell comprising at least one different nucleic acid molecule encoding a biparatopic antibody or a biparatopic binding antigen fragment antibody, in particular at least four different nucleic acid molecules encoding a biparatopic antibody or a biparatopic binding antigen fragment antibody. The invention further provides a cell-free expression system comprising at least one different nucleic acid molecule encoding a biparatopic antibody or a biparatopic binding antigen fragment antibody, in particular at least four different nucleic acid molecules encoding a biparatopic antibody or a biparatopic binding antigen fragment antibody.

The nucleic acids of the invention can be prepared or obtained in a manner known per se (e.g. by automated DNA synthesis and/or recombinant DNA technology), based on the information on the amino acid sequence for the alpha-synuclein biparatopic binding molecule of the invention given herein.

The nucleic acids of the invention can be prepared or obtained in a manner known per se (e.g. by automated DNA synthesis and/or recombinant DNA technology), based on the information on the amino acid sequence for the alpha-synuclein monospecific binding molecules of the invention given herein, and/or can be isolated from a suitable natural source.

For recombinant production of a biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof, nucleic acid encoding a biparatopic antibody or a functional fragment thereof, e.g., as described above, is isolated and inserted into one or more vectors for further cloning and/or expression in a host cell.

Suitable host cells for cloning or expression of antibody-encoding vectors include prokaryotic or eukaryotic cells described herein. In some embodiments, the host cell can be, but is not limited to, a Chinese Hamster Ovary (CHO) cell. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); mouse myeloma cells SP2/0-AG14 (ATCC CRL 1581; ATCC CRL 8287) or NS0 (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art. The term “host cell” generally refers to a cultured cell line. Accordingly, whole human beings into which an expression vector encoding an antigen binding polypeptide according to the invention has been introduced are explicitly excluded from the definition of a “host cell”. Cell-free expression systems may be based on use of cell lysates or extracts, such as CHO cell lysates (Stech, M., Nikolaeva, O., Thoring, L. et al. Cell-free synthesis of functional antibodies using a coupled in vitro transcription-translation system based on CHO cell lysates. Sci Rep 7, 12030 (2017)).

Examples of the vectors include M13 series vectors, pcDNA3 series vectors, pUC series vectors, pBR322, pBluescript, and pCR-Script. In addition to these vectors, for example, pGEM-T, pDIRECT, or pT7 can also be used for the purpose of cDNA subcloning and excision.

Particularly, expression vectors are useful for using the vectors for the purpose of producing the antibody or a functional fragment thereof. For example, when the host is E. coli such as JM109, DH5α, HB101, or XL1-Blue, the expression vectors indispensably have a promoter that permits efficient expression in E. coli, for example, lacZ promoter (Ward et al., Nature (1989) 341, 544-546; and FASEB J (1992) 6, 2422-2427), araB promoter (Better et al., Science (1988) 240, 1041-1043), or T7 promoter. Examples of such vectors include the vectors mentioned above as well as pGEX-5X-1 (manufactured by Pharmacia), “QIAexpress system” (manufactured by QIAGEN), pEGFP, and pET (in this case, the host is preferably BL21 expressing T7 RNA polymerase).

The vectors may contain a signal sequence for polypeptide secretion. In the case of production in the periplasm of E. coli, pelB signal sequence (Lei, S. P. et al., J. Bacteriol. (1987) 169, 4397) can be used as the signal sequence for polypeptide secretion. The vectors can be transferred to the host cells using, for example, calcium chloride methods or electroporation methods.

In addition to the E. coli expression vectors, examples of the vectors for producing the biparatopic antibody or a functional fragment thereof of the present invention include mammal-derived expression vectors (e.g., pcDNA3 (manufactured by Invitrogen Corp.), pEGF-BOS (Nucleic Acids. Res. 1990, 18(17), p 5322), pEF, and pCDM8), insect cell-derived expression vectors (e.g., “Bac-to-BAC baculovirus expression system” (manufactured by GIBCO BRL), and pBacPAK8), plant-derived expression vectors (e.g., pMH1 and pMH2), animal virus-derived expression vectors (e.g., pHSV, pMV, and pAdexLcw), retrovirus-derived expression vectors (e.g., pZIPneo), yeast-derived expression vectors (e.g., “Pichia Expression Kit” (manufactured by Invitrogen Corp.), pNV11, and SP-Q01), and Bacillus subtilis-derived expression vectors (e.g., pPL608 and pKTH50).

For the purpose of expression in animal cells such as CHO cells, HEK cells, COS cells, or NIH3T3 cells, the vectors indispensably have a promoter necessary for expression, for example, SV40 promoter (Mulligan et al., Nature (1979) 277, 108), MMTV-LTR promoter, EF1α promoter (Mizushima et al., Nucleic Acids Res (1990) 18, 5322), CAG promoter (Gene (1991) 108, 193), or CMV promoter and, more particularly, have a gene for screening for transformed cells (e.g., a drug resistance gene that can work as a marker by a drug (neomycin, G418, etc.)). Examples of the vectors having such properties include pMAM, pDR2, pBK-RSV, pBK-CMV, pOPRSV, and pOP13.

An exemplary method intended to stably express the gene and increase the number of intracellular gene copies involves transfecting CHO cells deficient in nucleic acid synthesis pathway with vectors having a DHFR gene serving as a complement thereto (e.g., pCHOI) and using methotrexate (MTX) in the gene amplification. An exemplary method intended to transiently express the gene involves using COS cells having a gene which expresses an SV40 T antigen on their chromosomes to transform the cells with vectors having a replication origin of SV40 (pcD, etc.). Also, a replication origin derived from polyomavirus, adenovirus, bovine papillomavirus (BPV), or the like may be used. The expression vectors for increasing the number of gene copies in a host cell system can additionally contain a selection marker such as an aminoglycoside transferase (APH) gene, a thymidine kinase (TK) gene, an E. coli xanthine guanine phosphoribosyltransferase (Ecogpt) gene, or a dihydrofolate reductase (dhfr) gene.

The biparatopic antibodies or functional fragments thereof of the present invention obtained by the methods described above can be isolated from inside host cells or from outside of the cells (the medium, or such), and purified to practically pure and homogeneous antibodies. The antibodies or functional fragments thereof can be separated and purified by methods routinely used for separating and purifying antibodies, and the type of method is not limited. For example, the antibodies or functional fragments thereof can be separated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectrofocusing, dialysis, recrystallization, and such.

The chromatographies include, for example, affinity chromatography, ion exchange chromatography, hydrophobic chromatography, gel filtration, reverse phase chromatography, and adsorption chromatography (Strategies for Protein Purification and Characterization: A Laboratory Course Manual. Ed Daniel R. Marshak et al., Cold Spring Harbor Laboratory Press, 1996). The chromatographic methods described above can be conducted using liquid-chromatography, for example, HPLC and FPLC. Resins used for affinity chromatography include protein A resins and protein G resins. Protein A based resins include, for example, Hyper D, POROS, and Sepharose FF (GE Amersham Biosciences). The present invention includes the biparatopic antibodies or functional fragments thereof that are highly purified using these purification methods.

The obtained biparatopic antibodies or functional fragments thereof can be purified to homogeneity. Separation and purification of the antibodies can be performed using separation and purification methods generally used for protein separation and purification. For example, the antibodies or functional fragments thereof can be separated and purified by appropriately selecting and combining column chromatography such as affinity chromatography, filtration, ultrafiltration, salting-out, dialysis, SDS-polyacrylamide gel electrophoresis, isoelectric focusing, and such, without limitation (Antibodies: A Laboratory Manual. Ed Harlow and David Lane, Cold Spring Harbor Laboratory, 1988). Resins used for affinity chromatography include, for example, protein A resins and protein G resins.

Several known approaches exist for delivering molecules across the blood brain barrier (BBB) and may be employed according to the invention. Non-limiting examples include alteration of the administration route, disruption of the BBB and alteration of its permeability, nanoparticle delivery, Trojan horse approaches, receptor-mediated transport, and cell and gene therapy.

Alteration of the administration route can be achieved by direct injection into the brain (see, e.g., Papanastassiou et al., Gene Therapy 9: 398-406(2002)), implanting a delivery device in the brain (see, e.g., Gillet al., Nature Med. 9: 589-595 (2003); and Gliadel Wafers™, Guildford Pharmaceutical), and intranasal administration to bypass the BBB (Mittal et al, Drug Deliv. 21(2):75-86. (2014))

Methods of barrier disruption include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416).

Methods of altering the BBB permeability include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), and inhibiting ABC drug transporters (see, e.g., U.S. Patent Application Publication No. 2003/0073713).

Trojan horse delivery methods of delivering the humanized antibody or humanized antibody fragment thereof across the blood brain barrier include, but are not limited to, cationizing the antibodies (see, e.g., U.S. Pat. No. 5,004,697), and the use of cell-penetration peptides such as Tat peptides to gain entry into the CNS. (see, e.g. Dietz et al., J. Neurochem. 104:757-765 (2008)).

Nanoparticle delivery methods of delivering the antibody or antigen-binding fragment thereof across the blood brain barrier include, but are not limited to, encapsulating the antibody or antigen-binding fragment thereof in delivery vehicles such as liposomes, or extracellular vesicles or exosomes, that are coupled to antibody or antigen-binding fragments or alternatively peptides that bind to receptors on the vascular endothelium of the blood-brain barrier (see, e.g., U.S. Patent Application Publication No. 20020025313), and coating the antibody or antigen-binding fragment thereof in low-density lipoprotein particles (see, e.g., U.S. Patent Application Publication No. 20040204354) or apolipoprotein E (see, e.g., U.S. Patent Application Publication No. 20040131692).

Alpha-synuclein antibodies of the invention can be further modified to enhance blood brain barrier penetration.

The alpha-synuclein antibody or antigen-binding fragment thereof of the invention can be fused to a polypeptide binding to a blood-brain barrier receptor. BBB receptors include, but are not limited to, a receptor transfer unit, transferrin receptor, insulin receptor or low-density lipoprotein receptor. The polypeptide can be any suitable polypeptide. It may, for example, comprise a peptide, a receptor ligand, a single domain antibody (VHH), a scFv or a Fab fragment.

The alpha-synuclein antibodies of the invention can also be delivered as a corresponding nucleic acid encoding the alpha-synuclein antibody. Such nucleic acid molecule may be a part of a viral vector for targeted delivery to the blood brain barrier or any other cell type in the CNS. A viral vector may be a recombinant adeno-associated viral vectors (rAAV) selected from any AAV serotype known in the art, including, without limitation, from AAV1 to AAV12 to enable the alpha-synuclein antibody or alpha-synuclein antibody fragment or alpha-synuclein antibody derivatives to be expressed intracellularly or into the brain parenchyma.

Cell therapy methods of delivering the alpha-synuclein antibody of the invention or the alpha-synuclein antibody fragment or alpha-synuclein antibody derivatives across the blood brain barrier include, but are not limited to, the use of the homing capacity of Endothelial Progenitor Cells (EPCs) transfected ex vivo with suitable vectors and the secretion and delivery of antibodies or antibody fragments to the brain by these cells (see, e.g., Heller et al., J Cell Mol Med. 00:1-7 (2020)), or the use of polymeric cell implant devices loaded with genetically engineered cells, to secrete antibodies or antibody fragments (see, e.g. Marroquin Belaunzaran et al. PLoS ONE 6(4): e18268 (2011)).

Biparatopic antibodies binding to a protein associated with a CNS disease, such as alpha-synuclein, Tau, TDP-43, ASC, NLRP3, C5a, C1q, C3, huntingtin or prion protein, in particular the alpha-synuclein biparatopic antibodies or functional fragments thereof provided herein may be identified, screened for, or characterized for their physical/chemical properties and/or biological activities by various assays known in the art.

In some embodiments, a biparatopic antibody or a functional fragment described herein is used as analytical reference, analytical standard, a tool compound or an in vitro screening tool.

In one aspect, a biparatopic antibody or a functional fragment thereof of the invention is tested for its antigen binding activity, e.g., by known methods such as ELISA, BIACore®, FACS, immunofluorescence or immunohistochemistry.

In another aspect, competition assays may be used to identify a biparatopic antibody or an antibody or a functional fragment thereof that competes with any of the biparatopic antibody or monospecific antibodies of the composition described herein for binding to aggregated or pathological alpha-synuclein. In certain embodiments, such a competing antibody binds to the same or similar epitope (e.g., a linear or a conformational epitope with total or partial overlap) that is bound by a biparatopic antibody or a functional fragment thereof described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

The invention also provides immunoconjugates comprising a biparatopic antibody binding to a protein associated with a CNS disease, such as alpha-synuclein, Tau, TDP-43, ASC, NLRP3, C5a, C1q, C3, huntingtin or prion protein, in particular an alpha-synuclein biparatopic antibody or a functional fragment thereof provided herein conjugated to one or more therapeutic agents, such as chemotherapeutic agents or drugs, growth inhibitory agents, toxins (e.g., protein toxins, enzymatically active toxins of bacterial, fungal, plant, or animal origin, or fragments thereof), radioactive isotopes (i.e., a radioconjugate), blood brain barrier penetration moieties or detectable labels. Immunoconjugates are also provided comprising mixtures of the invention. In such immunoconjugates one or more of the at least two monospecific antibodies or functional fragments thereof (preferably both of two monospecific antibodies or functional fragments thereof) is conjugated to one or more therapeutic agents, such as chemotherapeutic agents or drugs, growth inhibitory agents, toxins (e.g., protein toxins, enzymatically active toxins of bacterial, fungal, plant, or animal origin, or fragments thereof), radioactive isotopes (i.e., a radioconjugate), blood brain barrier penetration moieties or detectable labels.

In some embodiments, a labeled biparatopic antibody or a functional fragment thereof, is provided, comprising a biparatopic antibody or a functional fragment thereof described herein and a detectable label.

In some embodiments the alpha-synuclein biparatopic binding molecule of the present invention is linked to a detectable label.

In some embodiments, an immunoconjugate is provided, wherein the immunoconjugate comprises an isolated biparatopic antibody or a functional fragment thereof, described herein and a therapeutic agent.

In some embodiments the alpha-synuclein biparatopic binding molecule is part of an immunoconjugate wherein the alpha-synuclein biparatopic binding molecule is covalently linked to another suitable therapeutic agent.

In some embodiments, a conjugated biparatopic binding molecule, in particular biparatopic antibody or antigen-binding fragment thereof, is provided, comprising a biparatopic binding molecule, in particular a biparatopic antibody or antigen-binding fragment thereof, described herein and a conjugated molecule. Conjugates of the invention may be referred to as immunoconjugates. Any suitable conjugated molecule may be employed according to the invention. Suitable examples include, but are not limited to enzymes (e.g. alkaline phosphatase or horseradish peroxidase), avidin, streptavidin, biotin, Protein A/G, magnetic beads, fluorophores, radioactive isotopes (i.e., radioconjugates), nucleic acid molecules, detectable labels, therapeutic agents, toxins and blood brain barrier penetration moieties. Conjugation methods are well known in the art and several technologies are commercially available for conjugating antibodies to a label or other molecule. Conjugation is typically through amino acid residues contained within the binding molecules of the invention (such as lysine, histidine or cysteine). They may rely upon methods such as the NHS (Succinimidyl) ester method, isothiocyanate method, carbodiimide method and periodate method. Conjugation may be achieved through creation of fusion proteins for example. This is appropriate where the binding molecule is conjugated with another protein molecule. Thus, suitable genetic constructs may be formed that permit the expression of a fusion of the binding molecule of the invention with the label or other molecule. Nucleic acid molecules of the invention may, therefore, encode immunoconjugates in appropriate embodiments. Conjugation may be via a suitable linker moiety to ensure suitable spatial separation of the antibody and conjugated molecule, such as detectable label. However, a linker may not be required in all instances.

Various techniques exist for improving drug delivery across the blood-brain barrier (BBB) as discussed herein, which discussion applies mutatis mutandis. Non-invasive techniques include the so-called “Trojan horse approach” in which conjugated molecules deliver the binding molecules of the invention by binding to BBB receptors and mediating transport. Suitable molecules may comprise endogenous ligands or antibodies, in particular monoclonal antibodies, that bind specific epitopes on the BBB receptor.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease or disorder or abnormality, alleviation of symptoms, diminishment of any direct or indirect pathological consequences of the disease, preventing metastasis, decreasing the rate of disease progression, amelioration or palliation of the disease state, and remission or improved prognosis.

In some embodiments, biparatopic antibodies or functional fragments thereof of the invention are used to delay development of a disease or to slow the progression of a disease, disorder or abnormality. In particular embodiments, biparatopic antibodies or functional fragments thereof of the invention are for preventing, slowing down, halting, retaining and/or improving the motor capabilities or motor deficits, cognitive capabilities or cognitive deficits, or behavioral impairments of a subject suffering from a synucleopathy. In further particular embodiments, the biparatopic antibodies or functional fragments thereof of the invention are for improving motor capabilities, in particular facial expression, speech, ocular motor dysfunction, tremor at rest, action tremor, increased tone, rapid alternating movement of hands, finger tapping, leg agility, Heel-Shin test, arising from chair, posture, body sway and/or gait; improving cognitive deficits, in particular as measured by MoCA (Montreal Cognitive Assessment) or Addenbrookes Cognitive Examination; and/or improving behavioral impairments, in particular using NPI scale, wherein the synucleopathy is multiple system atrophy (MSA).

In a further embodiment, when the synucleopathy is Parkinson's disease, Multiple System Atrophy, Lewy Body dementia (LBD; including dementia with Lewy bodies (DLB) (“pure” Lewy body dementia), Parkinson's disease dementia (PDD)), or Diffuse Lewy Body Disease, the biparatopic antibodies or functional fragments thereof of the invention are for (i) improving motor capabilities, in particular activities of daily living (speech, salivation, swallowing, handwriting, cutting food and handling utensils, dressing, hygiene, turning in bed and adjusting bed clothes, falling, freezing when walking, walking, tremor, sensory complaints related to Parkinsonism), motor examination (speech, facial expression, tremor at rest, action or postural tremor of hands, rigidity, finger taps, hand movements, rapid alternating movements of hands, leg agility, arising from chair, posture, gait, postural stability, body bradykinesia and hypokinesia, dyskinesias, clinical fluctuations), symptomatic orthostatis, repeated falls and syncope, and/or transient unexplained loss of consciousness; and/or (ii) improving cognitive deficits; and/or (iii) improving behavioral impairments, in particular behavior and mood (intellectual Impairment, thought disorder, depression, motivation/initiative), delusions, hallucinations, agitation/aggression, depression/dysphoria, anxiety, elation/euphoria, apathy/indifference, irritability/lability, motor disturbance, nighttime behavior, and/or appetite/eating, deficits of attention, executive functions, visuospatial ability, visual hallucination; and/or (iv) improving rapid eye movement (REM) sleep disorders, in particular insomnia, hypersomnolence.

In one embodiment, a pharmaceutical composition is provided comprising at least one biparatopic antibody, or a functional fragment thereof, or a mixture comprising at least two monospecific antibodies or functional fragments thereof, as an active ingredient and a pharmaceutically acceptable carrier and/or excipient. In one embodiment, a pharmaceutical composition is provided comprising at least one biparatopic antibody, or a functional fragment thereof of the invention and at least one monospecific antibody described herein as an active ingredient and a pharmaceutically acceptable carrier and/or excipient. In one embodiment, a pharmaceutical composition is provided comprising at least two monospecific antibodies, or functional fragments thereof, as an active ingredient and a pharmaceutically acceptable carrier and/or excipient. For example, the biparatopic antibody, or a functional fragment thereof, or the at least two monospecific antibodies may be combined, as appropriate, with pharmaceutically acceptable carriers or media such as sterilized water or saline solution, vegetable oils, emulsifiers, suspensions, surfactants, stabilizers, flavoring agents, excipients, vehicles, preservatives, and binders, for example, and formulated into a pharmaceutical preparation. Examples of carriers include light anhydrous silicide acid, lactose, crystalline cellulose, mannitol, starch, cannellose calcium, carmellose sodium, hydroxypropylcellulose, hydroxypropylmethylcellulose, polyvinylacetal diethylaminoacetate, polyvinyl pyrrolidone, gelatin, medium chain fatty acid triglycerides, polyoxyethylene hydrogenated castor oil 60, sucrose, carboxymethyl cellulose, corn starch, and inorganic salts.

The amount of the active ingredient in these preparations can be set as appropriate within the designated range of doses.

In another embodiment, the present disclosure provides a product comprising at least (i) a container (e.g., an injection); (ii) a pharmaceutical composition comprising the biparatopic antibody or a functional fragment thereof or a mixture comprising at least two alpha-synuclein monospecific antibodies of the invention as an active ingredient(s) within the container; and (iii) a document instructing that the biparatopic antibody or a functional fragment thereof or a mixture comprising at least two alpha-synuclein monospecific antibodies of the invention should be administered according to a desired dosage regimen. Additionally, a label, a syringe, an injection needle, a pharmacologically acceptable medium, an alcohol cotton cloth, plaster, and the like may be additionally packaged, as appropriate, with this product. The container may be a bottle, a glass bottle, or a syringe, for example, and may be made of any of various materials such as glass and plastics. The container contains the pharmaceutical composition, and has an outlet sealed with a rubber stopper, for example. The container is provided with, for example, a label indicating that the pharmaceutical composition is for use in preventing or treating a selected pathological condition. In some cases, this label may describe the embodiment the biparatopic antibody or a functional fragment thereof or a mixture comprising at least two alpha-synuclein monospecific antibodies of the invention is used in combination with an additional therapeutic agent.

Biparatopic antibodies or immunoconjugates, mixtures of the invention can be used either alone or in combination with other agents in a therapy. For instance, a biparatopic antibody or immunoconjugate or a mixture comprising at least one biparatopic binding molecule and at least one alpha-synuclein monospecific binding molecule, or a mixture comprising at least two alpha-synuclein monospecific antibodies of the invention may be co-administered with at least one additional therapeutic agent. Such additional therapeutic agent is preferably selected from, but not limited to, neurological drugs, levodopa (e.g. Sinemet®), catechol-O-methyl transferase inhibitors (e.g. entacapone, tolcapone), dopamine agonists, monoamine oxidase B inhibitors (e.g. rasagiline, selegiline), amantadine, anticholinergic medication, anti-abeta antibodies, anti-Tau antibodies, Tau aggregation inhibitors, beta-amyloid aggregation inhibitors, anti-BACE1 antibodies, and BACE1 inhibitors.

A biparatopic antibody or a functional fragment thereof, immunoconjugate, monospecific antibodies of the mixtures of the invention (and any additional therapeutic agent) or pharmaceutical composition can be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including, but not limited to, single or multiple administrations over various time-points, bolus administration, and pulse infusion are contemplated herein.

The methods of the invention may comprise administering at least one additional therapy, preferably wherein the additional therapy is selected from, but not limited to, neurological drugs, levodopa (e.g. Sinemet®), catechol-O-methyl transferase inhibitors (e.g. entacapone, tolcapone), dopamine agonists, monoamine oxidase B inhibitors (e.g. rasagiline, selegiline), amantadine, anticholinergic medication, anti-abeta antibodies, anti-Tau antibodies, Tau aggregation inhibitors, beta-amyloid aggregation inhibitors, anti-BACE1 antibodies, and BACE1 inhibitors.

Biparatopic antibodies or functional fragments thereof, immunoconjugates, monospecific antibodies of the mixture, pharmaceutical compositions of the invention would be formulated, dosed, and administered in a fashion consistent with good medical practice. Factors for consideration in this context include the particular disease or disorder or abnormality being treated, the particular subject being treated, the clinical condition of the individual patient, the cause of the disease or disorder or abnormality, the site of delivery of the therapeutic agent, the method of administration, the scheduling of administration, and other factors known to medical practitioners. The biparatopic antibody or a functional fragment thereof, the monospecific antibodies of the mixture or immunoconjugate need not be, but is optionally formulated with one or more therapeutic agents currently used to prevent or treat the disease or disorder or abnormality in question. The effective amount of such other therapeutic agents depends on the amount of biparatopic antibody or a functional fragment thereof, monospecific antibodies of the mixture or immunoconjugate present in the formulation, the type of disease, or disorder or abnormality or treatment, and other factors discussed above. These are generally used in the same dosages and with administration routes as described herein, or about from 1 to 99% of the dosages described herein, or in any dosage and by any route that is empirically/clinically determined to be appropriate.

It is understood that any of the above formulations or therapeutic methods may be carried out using both an immunoconjugate of the invention and an alpha-synuclein biparatopic antibody or a functional fragment thereof and/or a mixture of alpha-synuclein monospecific antibodies and/or or a mixture comprising at least one biparatopic antibody or a functional fragment thereof and at least one alpha-synuclein monospecific antibodies or functional fragments thereof of the invention.

In some embodiments, a biparatopic antibody or a functional fragment thereof that binds to human alpha-synuclein is provided, wherein the biparatopic antibody or a functional fragment thereof binds extracellular or cytoplasmic alpha-synuclein. In some embodiments, a biparatopic antibody or a functional fragment thereof that binds to monomeric or aggregated alpha-synuclein is provided. In some embodiments of the invention, the monomeric, oligomeric or aggregated alpha-synuclein is post-translationally modified, e.g. phosphorylated or nitrosylated. The invention also relates to compositions comprising a biparatopic antibody or a functional fragment thereof (including derivatives thereof) or mixtures comprising at least two alpha-synuclein monospecific antibodies (including functional fragments thereof and derivatives thereof) as described herein and to therapeutic and diagnostic methods using such compositions for the prevention, diagnosis or treatment of a synucleopathy, wherein an effective amount of the antibody or a functional fragment thereof is administered to a patient in need thereof.

In certain embodiments, the alpha-synuclein biparatopic antibodies or functional fragments thereof or the compositions or the mixtures described herein are useful for detecting the presence of alpha-synuclein in a biological sample. In particular embodiments, the alpha-synuclein biparatopic antibodies or functional fragments thereof or the compositions or the mixtures described herein are useful for detecting the presence of aggregated and/or pathological alpha-synuclein, including, but not limited to, Lewy bodies, Lewy neurites and/or glial cytoplasmic inclusions in a biological sample. The term “detecting” as used herein encompasses quantitative or qualitative detection. Any suitable biological sample may be used, in particular a biological sample from a (human) subject that may contain alpha-synuclein. In certain embodiments, a biological sample comprises saliva, urine, nasal secretion, blood, brain and/or CSF, brain and/or interstitial fluid (ISF), more particularly a blood, brain and/or CSF or brain and/or ISF sample. Blood samples may be whole blood, serum or plasma samples for example, but are preferably plasma samples. In certain embodiments, a biological sample comprises a cell or tissue, such as cerebrospinal fluid (CSF), a cell or tissue of the brain (e.g., brain cortex or hippocampus), or blood. In some embodiments, a biological sample is cerebrospinal fluid.

In some embodiments, a biparatopic antibody binding to a protein associated with a CNS disease, such as alpha-synuclein, Tau, TDP-43, ASC, NLRP3, C5a, C1q, C3, huntingtin or prion protein, in particular an alpha-synuclein biparatopic antibody or functional fragments thereof or a mixture comprising at least two biparatopic antibodies binding to a protein associated with a CNS disease, such as alpha-synuclein, Tau, TDP-43, ASC, NLRP3, C5a, C1q, C3, huntingtin or prion protein, in particular alpha-synuclein monospecific antibodies for use in a method of diagnosis or detection is provided. In a further aspect, a method of detecting the presence of alpha-synuclein in a biological sample is provided. In certain embodiments, the method comprises contacting the biological sample with an alpha-synuclein biparatopic antibody or a functional fragment thereof or a mixture comprising at least two alpha-synuclein monospecific antibodies as described herein under conditions permissive for binding of the alpha-synuclein biparatopic antibody or a functional fragment thereof or the alpha-synuclein antibodies of the mixtures to alpha-synuclein, and detecting whether a complex is formed between the biparatopic alpha-synuclein antibody or a functional fragment thereof and alpha-synuclein, or between at least one of the monospecific antibodies of the mixture and alpha-synuclein. Such method may be an in vitro or in vivo method. Further, the complex formed between the alpha-synuclein biparatopic antibody or a functional fragment thereof and alpha-synuclein, or between at least one of the monospecific antibodies of the mixtures and alpha-synuclein in a test biological sample can be compared to the complex formed in a control biological sample (e.g., a biological sample from a healthy subject or subjects). The amount of the complex formed between the alpha-synuclein biparatopic antibody or a functional fragment thereof and alpha-synuclein, or between at least one of the monospecific antibodies of the mixture and alpha-synuclein in a test biological sample can also be quantified and compared to the amount of the complex formed in a control biological sample (e.g., a biological sample from a healthy subject or subjects) or to the average amount of the complex known to be formed in healthy subjects.

In certain embodiments, an alpha-synuclein binding molecule, in particular an alpha-synuclein antibody or antigen-binding fragment thereof, of the invention and as provided herein is useful for detecting the presence of alpha-synuclein in a biological sample. The disclosure is applicable to both biparatopic antibodies and fragments thereof, and to mixtures as described herein. In particular embodiments, the alpha-synuclein binding molecule, in particular an alpha-synuclein antibody or antigen-binding fragment thereof, of the invention and as provided herein is useful as an assay reagent, positive control, biomarker detection reagent and/or calibrator for an immunoassay, (including, but not limited to an ELISA, MSD (Meso Scale Discovery Inc., USA), Luminex (Luminex Corp., USA), Alphalisa (PerkinElmer, Inc., USA), Gyrolab (Gyros Protein Technologies AB, Sweden), Simoa (Quanterix Corp., USA), Gyros™ (Given et al., 2012), Singulex Erenna (EMD Millipore, Corp., USA), iR-SENSE/Immuno-InfraRed assay (Nabers et al, 2016), MITOMI (Piraino et al, 2016), Immunoprecipitation combined with liquid chromatography mass spectrometry (IP LC-MS/MS; Shimadzu, Germany), Surface plasmon resonance (SPR; Cytiva Europe, Switzerland), Atomic force microscope (AFM) (Kiio and Park, 2020) or any other assay technology or kit that relies on antibodies for target immunocapture and/or detection). As such, the alpha-synuclein binding molecule, in particular the alpha-synuclein antibody or antigen-binding fragments thereof, may be used in assays for validating/screening alpha-synuclein binding molecules, alpha-synuclein antibodies or antigen-binding fragments thereof. The alpha-synuclein binding molecules, in particular an alpha-synuclein antibody or antigen-binding fragment thereof, of the invention may be used as detection tools and/or positive controls as they bind to all alpha-synuclein species in the sample in selective fashion. Diagnostic compositions of the invention may be used in such methods.

The invention therefore provides a method of detecting alpha-synuclein in a sample obtained from a subject, the method comprising contacting the sample with a binding molecule, in particular an antibody or antigen-binding fragment of the invention and detecting binding of the antibody or antigen-binding fragment thereof in order to detect alpha-synuclein in the sample. The disclosure is applicable to both biparatopic antibodies and fragments and to mixtures as described herein. The methods of the invention may detect any useful form of alpha-synuclein as described herein. Thus, the methods may permit detection of aggregated and/or pathological alpha-synuclein, including, but not limited to, Lewy bodies, Lewy neurites and/or glial cytoplasmic inclusions.

Similarly, the invention provides a method of quantifying alpha-synuclein in a sample obtained from a subject, the method comprising contacting the sample with a binding molecule, in particular an antibody or antigen-binding fragment of the invention and performing quantification based on the binding of the binding molecule to alpha-synuclein. The disclosure is applicable to both biparatopic antibodies and fragments and to mixtures as described herein. This method may comprise comparing the alpha-synuclein levels in the sample to those in a control sample or samples. The levels in control samples represent known levels against which the levels in the test sample may be determined. The control samples are not, therefore, necessarily tested at the same time as the method of quantification is performed. However, in some embodiments, reference levels are determined in parallel with the test sample. For example, a quantitative ELISA, ELISA, MSD (Meso Scale Discovery Inc., USA), Luminex (Luminex Corp., USA), Alphalisa (PerkinElmer, Inc., USA), Gyrolab (Gyros Protein Technologies AB, Sweden), Simoa (Quanterix Corp., USA), Gyros™ (Given et al., 2012), Singulex Erenna (EMD Millipore, Corp., USA), iR-SENSE/Immuno-InfraRed assay (Nabers et al, 2016), MITOMI (Piraino et al, 2016), Immunoprecipitation combined with liquid chromatography mass spectrometry (IP LC-MS/MS; Shimadzu, Germany), Surface plasmon resonance (SPR; Cytiva Europe, Switzerland), Atomic force microscope (AFM) (Kiio and Park, 2020) may be performed. A standard curve may be generated to permit quantification based on a dilution series (serial dilution) of alpha-synuclein. Diagnostic compositions of the invention may be used in such methods. Sandwich immunoassays, incorporating a suitable capture and detection antibody or antigen binding fragment thereof, may be used in the methods of quantifying alpha-synuclein in a sample obtained from a subject.

The invention also provides a method for diagnosing a disease, disorder and/or condition associated with alpha-synuclein comprising contacting the sample with a binding molecule, in particular an antibody or antigen-binding fragment of the invention and comparing the alpha-synuclein levels in the sample to those in a control sample or samples. Higher levels of alpha-synuclein in the sample compared with a control level based on healthy subjects are indicative of a disease, disorder and/or condition associated with alpha-synuclein. Additionally or alternatively similar or higher levels of alpha-synuclein in the sample compared with a diseased control (i.e. one or more samples from a subject having the disease, disorder and/or condition associated with alpha-synuclein) are indicative of a disease, disorder and/or condition associated with alpha-synuclein. Diagnostic compositions of the invention may be used in such methods. The disclosure is applicable to both biparatopic antibodies and fragments and to mixtures as described herein. Sandwich immunoassays, incorporating a suitable capture and detection antibody or antigen binding fragment thereof, may be used in the methods of diagnosing a disease, disorder and/or condition associated with alpha-synuclein.

The binding molecules of the invention are also useful in classification methods, for example, to indicate the relative stage of the disease, disorder and/or condition associated with alpha-synuclein. The invention therefore also provides a method for classifying a disease, disorder and/or condition associated with alpha-synuclein comprising contacting a sample from a subject with a binding molecule, in particular an antibody or antigen-binding fragment of the invention and comparing the alpha-synuclein levels in the sample to those in a control sample or samples in order to classify the disease. A range of controls representative of different classes of disease may be employed in order to classify the sample. The test sample may be classified based on the best match to the control samples. Higher levels of alpha-synuclein in the sample compared with a control level based on healthy subjects are indicative of a disease, disorder and/or condition associated with alpha-synuclein. Similar or higher levels of alpha-synuclein in the sample compared with a diseased control at a certain stage of disease are indicative of that stage of disease, disorder and/or condition associated with alpha-synuclein. Such methods may be performed in relation to subjects known to have the disease, disorder and/or condition associated with alpha-synuclein and/or in relation to subjects not already known to have the disease, disorder and/or condition associated with alpha-synuclein. Diagnostic compositions of the invention may be used in such methods. The disclosure is applicable to both biparatopic antibodies and fragments, and to mixtures as described herein. Sandwich immunoassays, incorporating a suitable capture and detection antibody or antigen binding fragment thereof, may be used in the classification methods of the invention.

The invention also provides a method for monitoring a disease, disorder and/or condition associated with alpha-synuclein at two or more time points using samples from a subject, the method comprising contacting the samples with a binding molecule, in particular an antibody or antigen-binding fragment of the invention and comparing the alpha-synuclein levels in the samples, wherein higher levels of alpha-synuclein in the later sample compared with one or more earlier samples are indicative of progression of a disease, disorder and/or condition associated with alpha-synuclein. Similarly, the invention provides a method for monitoring a disease, disorder and/or condition associated with alpha-synuclein at two or more time points using samples from a subject, the method comprising contacting the samples with a binding molecule, in particular an antibody or antigen-binding fragment of the invention and comparing the alpha-synuclein levels in the samples, wherein lower levels of alpha-synuclein in the later sample compared with one or more earlier samples are indicative of regression of a disease, disorder and/or condition associated with alpha-synuclein. These methods also permit a lack of progression of the disease to be monitored, where there is no significant change in levels of alpha-synuclein in the later sample compared with one or more earlier samples. Such methods are typically performed in relation to subjects known to have the disease, disorder and/or condition associated with alpha-synuclein. Diagnostic compositions of the invention may be used in such methods. The disclosure is applicable to both biparatopic antibodies and fragments and to mixtures as described herein. Sandwich immunoassays, incorporating a suitable capture and detection antibody or antigen binding fragment thereof, may be used in the monitoring methods of the invention.

Monitoring methods are useful to determine whether a particular therapy is successful or otherwise. The invention therefore also provides a method for monitoring a disease, disorder and/or condition associated with alpha-synuclein at two or more time points using samples from a subject, the method comprising contacting the samples with a binding molecule, in particular an antibody or antigen-binding fragment of the invention, wherein lower levels of alpha-synuclein in the later sample compared with one or more earlier samples are indicative of successful treatment of a disease, disorder and/or condition associated with alpha-synuclein. The therapy may be any suitable candidate therapeutic agent, such as an antibody or small molecule therapeutic. These methods also permit a lack of progression of the disease to be monitored, where there is no significant change in levels of alpha-synuclein in the later sample compared with one or more earlier samples. This may also be considered successful treatment in some circumstances. Indeed, a decline in the rate of increase of alpha-synuclein levels between samples, compared with the rate of increase prior to therapy, may also be considered indicative of successful treatment. Such methods are typically performed in relation to subjects known to have the disease, disorder and/or condition associated with alpha-synuclein. Unsuccessful treatment may be determined where the treatment provides no decline in the rate of increase of alpha-synuclein levels between samples, compared with the rate of increase prior to therapy. Diagnostic compositions of the invention may be used in such methods. The disclosure is applicable to both biparatopic antibodies and fragments and to mixtures as described herein. Sandwich immunoassays, incorporating a suitable capture and detection antibody or antigen binding fragment thereof, may be used in the methods of monitoring therapy of the invention.

The binding molecules of the invention may also be used to assist with therapy selection. Thus, the invention provides a method for selecting a therapy for treatment of a disease, disorder and/or condition associated with alpha-synuclein, the method comprising contacting samples taken before and after treatment with a binding molecule, in particular an antibody or antigen-binding fragment of the invention, wherein lower levels of alpha-synuclein in the sample taken after treatment compared with the sample taken before treatment are indicative of successful treatment of a disease, disorder and/or condition associated with alpha-synuclein and thus the therapy is selected for treatment. The therapy may be any suitable candidate therapeutic agent, such as an antibody or small molecule therapeutic. A therapy halting progression of the disease may also be selected, where there is no significant change in levels of alpha-synuclein in the later sample compared with one or more earlier samples. This may also be considered successful treatment in some circumstances. Indeed, a decline in the rate of increase of alpha-synuclein levels between samples, compared with the rate of increase prior to therapy, may also be considered indicative of successful treatment and therefore result in selection of the particular therapy. Such methods are typically performed in relation to subjects known to have the disease, disorder and/or condition associated with alpha-synuclein. Unsuccessful treatment may be determined where the treatment provides no decline in the rate of increase of alpha-synuclein levels between samples, compared with the rate of increase prior to therapy. Such therapy is not selected for treatment. Alternatively, higher levels of alpha-synuclein in the sample taken after treatment compared with the sample taken before may be indicative of unsuccessful treatment of a disease, disorder and/or condition associated with alpha-synuclein and thus the therapy is not selected for treatment. Diagnostic compositions of the invention may be used in such methods. The disclosure is applicable to both biparatopic antibodies and fragments and to mixtures as described herein. Sandwich immunoassays, incorporating a suitable capture and detection antibody or antigen binding fragment thereof, may be used in the therapy selection methods of the invention (as applied to individual subjects).

Methods of the invention are also useful to determine whether a particular therapy is successful or otherwise in the context of a larger, controlled study, such as a clinical trial. Thus, these methods are typically applied to a treatment group of subjects that is compared with a group of subjects not treated with the therapy. In such a context, control samples not treated with the therapy are also available for comparative purposed (placebo group). The invention therefore also provides a method for assessing a candidate therapy for a disease, disorder and/or condition associated with alpha-synuclein, the method comprising, following treatment of one or more subjects, contacting samples from the one or more treated subjects with a binding molecule, in particular an antibody or antigen-binding fragment of the invention, wherein lower levels of alpha-synuclein in the samples compared with levels in corresponding samples from subjects not treated with the therapy are indicative of successful treatment of a disease, disorder and/or condition associated with alpha-synuclein. The methods are typically performed in relation to a plurality (i.e. at least two) treated subjects and a plurality of control subjects. The treated and control groups may or may not be of the same size. They may each comprise 3 or more, 4 or more, 5 or more, 10 or more, 20 or more, 50 or more subjects in some embodiments. The therapy may be any suitable candidate therapeutic agent, such as a biologic, in particular an antibody, a vaccine or small molecule therapeutic. The methods may be performed at multiple time points in matched samples between the treatment and placebo groups in order to monitor the effectiveness of the candidate therapy over a defined time period. An initial pre-therapy sample is typically also taken. Thus, the methods may comprise contacting samples from the one or more treated subjects and the subjects not treated with the therapy with a binding molecule, in particular an antibody or antigen-binding fragment of the invention prior to treatment to determine base levels of alpha-synuclein. “Prior to treatment” means prior to administration of the therapy or the placebo depending upon the subject group. The binding molecules of the invention may therefore also be used to assist with assessment of candidate therapies in the context of clinical trials. Candidate therapies providing successful treatment may be selected and, ultimately, approved for marketing. Diagnostic compositions of the invention may be used in such methods. The disclosure is applicable to both biparatopic antibodies and fragments and to mixtures as described herein. Sandwich immunoassays, incorporating a suitable capture and detection antibody or antigen binding fragment thereof, may be used in the therapy selection methods of the invention (as applied to clinical trials).

In some embodiments, an alpha-synuclein biparatopic antibody or a functional fragment thereof is used to select subjects eligible for therapy with an alpha-synuclein biparatopic antibody or a functional fragment thereof, e.g. where alpha-synuclein is a biomarker for selection of patients. For example, in some embodiments, an alpha-synuclein biparatopic antibody or a functional fragment thereof is used to detect whether the subject has a disease, disorder or abnormality associated with alpha-synuclein aggregates including but not limited to, Lewy bodies, Lewy neurites and/or Glial cytoplasic inclusions, or whether the subject is at high risk (or predisposed to) a disease or disorder or abnormality associated with alpha-synuclein aggregates including but not limited to, Lewy bodies, Lewy neurites and/or Glial cytoplasic inclusions.

Exemplary diseases or disorders or abnormality that may be diagnosed, prevented or treated using a biparatopic antibody or a functional fragment thereof of the invention or a mixture comprising at least one biparatopic antibody or a functional fragment thereof and at least one alpha-synuclein monospecific antibodies or functional fragments thereof or a mixture comprising at least two alpha-synuclein monospecific antibodies of the invention include diseases or disorders or abnormalities associated with alpha-synuclein aggregates including, but not limited to, Parkinson's disease (sporadic, familial with alpha-synuclein mutations, familial with mutations other than alpha-synuclein, pure autonomic failure and Lewy body dysphagia), Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

In some embodiments, an immunoconjugate is provided, wherein the immunoconjugate comprises an isolated alpha-synuclein biparatopic antibody or a functional fragment thereof described herein and a therapeutic agent.

In some embodiments, a labeled antibody is provided, comprising an alpha-synuclein biparatopic antibody or a functional fragment thereof described herein and a detectable label.

In some embodiments the alpha-synuclein biparatopic antibody or a functional fragment thereof of the present invention is linked to a detectable label.

In some embodiments the alpha-synuclein biparatopic antibody or a functional fragment thereof is part of an immunoconjugate wherein the alpha-synuclein binding molecule is covalently linked to another suitable therapeutic agent.

In some embodiments an alpha-synuclein biparatopic antibody or a functional fragment thereof is part of a pharmaceutical composition comprising an alpha-synuclein biparatopic antibody or a functional fragment thereof, or an immunoconjugate wherein the alpha-synuclein biparatopic antibody or a functional fragment thereof is covalently linked to another suitable therapeutic agent, or a composition comprising an alpha-synuclein biparatopic antibody or a functional fragment thereof specific binding molecule combined with a pharmaceutically acceptable carrier and/or excipient.

In some embodiments an alpha-synuclein biparatopic antibody or a functional fragment thereof is part of a diagnostic kit comprising an an alpha-synuclein biparatopic antibody or a functional fragment thereof, or an immunoconjugate wherein the an alpha-synuclein biparatopic antibody or a functional fragment thereof is covalently linked to another suitable therapeutic agent, or a composition comprising an alpha-synuclein biparatopic antibody or a functional fragment thereof.

In some embodiments an an alpha-synuclein biparatopic antibody or a functional fragment thereof is used in an immunodiagnostic method for use in the prevention, diagnosis, alleviation of symptoms associated with, or treatment of a disease or disorder or abnormality associated with alpha-synuclein aggregates including, but not limited to, Lewy bodies, Lewy neurites, and/or glial cytoplasmic inclusions.

In some embodiments, a diagnostic composition is provided, comprising an isolated an alpha-synuclein biparatopic antibody or a functional fragment thereof, described herein and a pharmaceutically acceptable carrier and/or excipient.

Pharmaceutical formulations of an an alpha-synuclein biparatopic antibody or a functional fragment thereof or diagnostic composition as described herein are prepared by mixing such antibody or diagnostic composition having the desired degree of purity with one or more optional pharmaceutically acceptable carriers and/or excipients and/or diluents (Remington's Pharmaceutical Sciences 16th edition, Osol, A. Ed. (1980)). Typically, the antibody or fragment therefor is prepared as a lyophilized formulation or aqueous solution. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. Pharmaceutically acceptable excipients that may be used to formulate the compositions include, but are not limited to: ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and lanolin. Diluents may be buffers. They may comprise a salt selected from the group consisting of phosphate, acetate, citrate, succinate and tartrate, and/or wherein the buffer comprises histidine, glycine, TRIS glycine, Tris, or mixtures thereof. It is further envisaged in the context of the present invention that the diluent is a buffer selected from the group consisting of potassium phosphate, acetic acid/sodium acetate, citric acid/sodium citrate, succinic acid/sodium succinate, tartaric acid/sodium tartrate, and histidine/histidine HCl or mixtures thereof.

In some embodiments a biparatopic antibody binding to a protein associated with a CNS disease, such as alpha-synuclein, Tau, TDP-43, ASC, NLRP3, C5a, C1q, C3, huntingtin or prion protein, in particular an alpha-synuclein biparatopic antibody or a functional fragment thereof is part of a diagnostic kit comprising a biparatopic antibody binding to a protein associated with a CNS disease, such as alpha-synuclein, Tau, TDP-43, ASC, NLRP3, C5a, C1q, C3, huntingtin or prion protein, in particular an alpha-synuclein biparatopic antibody or a functional fragment thereof, or an immunoconjugate wherein the biparatopic antibody binding to a protein associated with a CNS disease, such as alpha-synuclein, Tau, TDP-43, ASC, NLRP3, C5a, C1q, C3, huntingtin or prion protein, in particular the alpha-synuclein biparatopic antibody or a functional fragment thereof described herein.

In some embodiments an alpha-synuclein biparatopic antibody or a functional fragment thereof or a mixture comprising at least one biparatopic antibody or a functional fragment thereof and at least one alpha-synuclein monospecific antibodies or functional fragments thereof, or a mixture comprising at least two alpha-synuclein monospecific antibodies or functional fragments thereof is part of a method for the prevention, alleviation of symptoms associated with, or treatment of a synucleinopathy.

In some embodiments a biparatopic antibody binding to a protein associated with a CNS disease, such as alpha-synuclein, Tau, TDP-43, ASC, NLRP3, C5a, C1q, C3, huntingtin or prion protein, in particular an alpha-synuclein biparatopic antibody or a functional fragment thereof is used in a method for diagnosing presymptomatic disease or disorder or abnormality, or for monitoring disease or disorder or abnormality progression and therapeutic efficacy of a therapeutic agent, or for predicting responsiveness, or for selecting patients which are likely to respond to the treatment with a biparatopic antibody binding to a protein associated with a CNS disease, such as alpha-synuclein, Tau, TDP-43, ASC, NLRP3, C5a, C1q, C3, huntingtin or prion protein, in particular alpha-synuclein biparatopic antibody or a functional fragment thereof or a mixture comprising at least two biparatopic antibodies binding to a protein associated with a CNS disease, such as alpha-synuclein, Tau, TDP-43, ASC, NLRP3, C5a, C1q, C3, huntingtin or prion protein, in particular alpha-synuclein monospecific antibodies or functional fragments thereof of the invention. Said method is preferably performed using a sample of human blood or urine. Most preferably the method involves an ELISA-based or surface adapted assay.

The invention furthermore relates to a method of detecting aggregated and/or pathological alpha-synuclein, including, but not limited to, Lewy neurites, Lewy Bodies and/or Glial cytoplasmic inclusions, comprising contacting a sample with the biparatopic binding molecule or the mixture of the invention, particularly wherein the sample is a brain sample, an interstitial fluid (ISF), a cerebrospinal fluid sample, urine sample or a blood sample.

In some embodiments an alpha-synuclein biparatopic antibody or a functional fragment thereof, or a mixture comprising at least one biparatopic antibody or a functional fragment thereof, or a mixture comprising at least two alpha-synuclein monospecific antibodies or functional fragments thereof is used in a method wherein the antibody or the functional fragment thereof is contacted with a sample (e.g., blood, interstitial fluid, cerebrospinal fluid, or brain tissue) to detect, diagnose a disease or disorder or abnormality associated with alpha-synuclein aggregates, such as Parkinson's disease (sporadic, familial with alpha-synuclein mutations, familial with mutations other than alpha-synuclein, pure autonomic failure and Lewy body dysphagia), Parkinson's disease with dementia, Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder, more particularly Parkinson's Disease, Multiple System Atrophy, Lewy Body dementia (LBD; including dementia with Lewy bodies (DLB) (“pure” Lewy body dementia), Parkinson's disease dementia (PDD)), or Diffuse Lewy Body Disease.

In another embodiment, an alpha-synuclein biparatopic antibody or a functional fragment thereof, or a mixture comprising at least one biparatopic antibody or a functional fragment thereof, or a mixture comprising at least two alpha-synuclein monospecific antibodies or functional fragments thereof is used to detect, diagnose or monitor a disease, disorder or abnormality associated with alpha-synuclein aggregates selected from Parkinson's disease (sporadic, familial with alpha-synuclein mutations, familial with mutations other than alpha-synuclein, pure autonomic failure and Lewy body dysphagia), Parkinson's disease with dementia, Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17, and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder; more particularly Parkinson's Disease, Multiple System Atrophy, Lewy Body dementia (LBD; including dementia with Lewy bodies (DLB) (“pure” Lewy body dementia), Parkinson's disease dementia (PDD)), or Diffuse Lewy Body Disease.

In some embodiments, an alpha-synuclein biparatopic antibody or a functional fragment thereof, or a mixture comprising at least one biparatopic antibody or a functional fragment thereof, or an immunoconjugate, or a mixture comprising at least two alpha-synuclein monospecific antibodies for use as a medicament is provided.

In some embodiments, an alpha-synuclein biparatopic antibody or a functional fragment thereof, or a mixture comprising at least one biparatopic antibody or a functional fragment thereof, or an immunoconjugate, or a mixture comprising at least two alpha-synuclein monospecific antibodies for the manufacture or preparation of a medicament.

In another aspect of the invention, an article of manufacture containing materials useful for the treatment, prevention and/or diagnosis of the disease or disorders or abnormality described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disease, disorder or abnormality and may have a sterile access port (for example the container may be an intravenous solution bag or a vial having a stopper pierceable by a hypodermic injection needle). At least one active agent in the composition is a biparatopic antibody or functional fragment thereof or immunoconjugate or at least two alpha-synuclein monospecific antibodies of the invention. The label or package insert indicates that the composition is used for treating the condition of choice. Moreover, the article of manufacture may comprise (a) a first container with a composition contained therein, wherein the composition comprises a biparatopic antibody or immunoconjugate or at least two monospecific antibodies of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

### BRIEF DESCRIPTION OF FIGURES

FIG. 1: Antibody binding to human full-length recombinant alpha-synuclein. Binding to recombinant full-length alpha-synuclein for the antibodies derived from stable hybridoma clones was determined using an indirect ELISA. Antibodies were diluted from 1 μg/mL to 0.0005 μg/mL. Results are expressed in optical densities (O.D.), mean values of two technical replicates t SEM are shown. Commercial antibody Syn1 was used as a positive control.

FIG. 2. Epitope mapping on alpha-synuclein. Epitope mapping for the antibodies derived from stable hybridoma clones was determined using an indirect ELISA on a library of 15-mer peptides covering the entire sequence of human alpha-synuclein from 1 to 140aa. (A) Results on peptides from 1 to 69aa and full-length alpha-synuclein. (B) Results on peptides from 64 to 140aa and full-length alpha-synuclein. Results are expressed as optical density (O.D.). Each bar represents data for individual antibody. Amino-acid sequence of alpha-synuclein indicated as shown in Table 3.

FIG. 3: Effect of mAbs on aggregation half-times in seeded a-syn aggregation. (A) Change in τ_{1/2} values, relative to no mAb control, from in vitro alpha-synuclein aggregations in the presence of the indicated mAbs at 3.28 μM. Error bars represent calculated SEM. Significance was determined using a one-way ANOVA (Dunnett's multiple comparisons test) versus aggregation with no antibody (no mAb) (n.s. not significant; (*) P<0.033; (***) P<0.001). (B) Percent increases of τ_{1/2} values, relative to the absence of antibody, are plotted for the seeded aggregations in the presence of the indicated mAb. Error bars represent the propagation of error (Equation 5). Significance was determined using a one-way ANOVA (Dunnett's multiple comparisons test) versus aggregation with IgG2a control Ab (n.s. not significant; (*) P<0.033; (***) P<0.001).

FIG. 4: Single-cycle kinetic sensograms of alpha-synuclein antibody responses to monomeric or fibrillar alpha-synuclein. (A) Sensogram from single-cycle kinetics of monomeric alpha-synuclein of ACI-7067-1101C8-Ab2 (black trace). (B) Sensogram from single-cycle kinetics of monomeric alpha-synuclein of ACI-7067-1113D10-Ab1 (black trace). (C) Sensogram from single-cycle kinetics of fibrillar alpha-synuclein of ACI-7067-1101C8-Ab2 (black trace). (D) Sensogram from single-cycle kinetics of fibrillar alpha-synuclein of ACI-7067-1113D10-Ab1 (black trace). 1:1 binding fits using a homogenous Langmuir model are shown overlaid (gray traces).

FIG. 5: Target engagement of alpha-synuclein antibodies in tissues from PD and MSA cases. (A) Representative images of immunostaining with alpha-synuclein antibodies for the detection of pathological alpha-synuclein aggregates in brain tissue from PD amygdala and (B) the medulla oblongata of a MSA case. An antibody recognizing alpha-synuclein phosphorylated at Ser129, (pSyn) used as control for detecting pathological aggregated and phosphorylated alpha-synuclein.

FIG. 6: Epitope mapping on alpha-synuclein. Epitope mapping for the antibodies derived from stable hybridoma clones was determined using an indirect ELISA on a library of 15-mer peptides covering the entire sequence of human alpha-synuclein from 1 to 140aa. (A) Results on peptides from 1 to 69aa and full-length alpha-synuclein. (B) Results on peptides from 64 to 140aa and full-length alpha-synuclein. Results are expressed as optical density (O.D.). Each bar represents data for individual antibody. Amino-acid sequence of alpha-synuclein indicated as shown in Table 3.

FIG. 7: Effect of mAbs on aggregation half-times in seeded a-syn aggregation. (A) Change in τ_{1/2} values, relative to no mAb control, from in vitro alpha-synuclein aggregations in the presence of the indicated mAbs at 3.28 μM. Error bars represent calculated SEM. Significance was determined using a one-way ANOVA (Dunnett's multiple comparisons test) versus aggregation with no antibody (no mAb) ((****) P<0.0001). (B) Percent increases of τ_{1/2} values, relative to the absence of antibody, are plotted for the seeded aggregations in the presence of the indicated mAb. Error bars represent the propagation of error (Equation 5). Significance was determined using a one-way ANOVA (Dunnett's multiple comparisons test) versus aggregation with no antibody control (n.s. not significant; (**) P<0.01; (***) P<0.0008, (****) P<0.0001).

FIG. 8: Inhibition or delay of seeded alpha-synuclein aggregation by monoclonal antibodies tested in combination/mixture. Seeded alpha-synuclein aggregation in vitro is monitored by measuring thioflavin T (ThT) fluorescence. Mean values of aggregation kinetics derived from ThT fluorescence signal of triplicate measurements overtime in hours (h) are shown. The aggregation of alpha-synuclein in the absence of antibodies (No antibody, black continuous line) is overlayed to the kinetic traces of the aggregation of alpha-synuclein in the presence of the indicated antibodies at 1.64 μM (doted lines) or a combination of the same antibodies at 3.28 μM (gray continuous line). (A) Antibodies tested were ACI-7067-1101C8-Ab2 binding to epitope 124-131 in the C-terminus and ACI-7067-1108B11-Ab2 binding to epitope 131-140 also in the C-terminus. (B) Antibodies tested were ACI-7067-1101C8-Ab2 binding to epitope 124-131 in the C-terminus and ACI-7067-1113D10-Ab1 binding to epitope 128-135 also in the C-terminus. (C) Antibodies tested were ACI-7067-1101C8-Ab2 binding to epitope 124-131 in the C-terminus and ACI-7067-1108H1-Ab1 binding to epitope 65-74 in the NAC domain. (D) Antibodies tested were ACI-7067-1113D10-Ab1 binding to epitope 128-135 in the C-terminus and ACI-7067-1108H1-Ab1 binding to epitope 65-74 in the NAC domain.

FIG. 9: Inhibition or delay of seeded alpha-synuclein aggregation by antibody binding (Fab) fragments tested in combination/mixture. Seeded alpha-synuclein aggregation in vitro is monitored by measuring thioflavin T (ThT) fluorescence. Mean values of normalized aggregation kinetics derived from ThT fluorescence signal of triplicate measurements over time in hours (h) are shown. The aggregation of alpha-synuclein in the absence of Fab fragments (No antibody, black continuous line) is overlayed to the kinetic traces of the aggregation of alpha-synuclein in the presence of the indicated Fabs at 1.64 μM (doted lines) or a combination of the same Fabs at 3.28 μM (gray continuous line). (A) Fabs tested were Fab ACI-7067-1101C8-Ab2 binding to epitope 124-131 in the C-terminus and Fab ACI-7067-1108B11-Ab2 binding to epitope 131-140 also in the C-terminus. (B) Fabs tested were Fab ACI-7067-1101C8-Ab2 binding to epitope 124-131 in the C-terminus and Fab ACI-7067-1113D10-Ab1 binding to epitope 128-135 also in the C-terminus. (C) Fabs tested were Fab ACI-7067-1101C8-Ab2 binding to epitope 124-131 in the C-terminus and Fab ACI-7067-1108H1-Ab1 binding to epitope 65-74 in the NAC domain. (D) Fabs tested were Fab ACI-7067-1113D10-Ab1 binding to epitope 128-135 in the C-terminus and Fab ACI-7067-1108H1-Ab1 binding to epitope 65-74 in the NAC domain.

FIG. 10-13: Effect of alpha-synuclein antibodies (mAbs) on aggregation half-times in seeded a-syn aggregation. (A) Change in τ_{1/2} values, relative to no mAb control, from in vitro alpha-synuclein aggregations in the presence of the indicated mAbs at 3.28 μM. Error bars represent calculated SEM. Significance was determined using a one-way ANOVA (Dunnett's multiple comparisons test) versus aggregation with no antibody (no mAb) ((****) P<0.0001). (B) Percent increases of τ_{1/2} values, relative to the absence of antibody, are plotted for the seeded aggregations in the presence of the indicated mAb. Error bars represent the propagation of error (Equation 5). Significance was determined using a one-way ANOVA (Dunnett's multiple comparisons test) versus aggregation with no antibody control (n.s. not significant; (**) P<0.01; (***) P<0.0008, (****) P<0.0001).

FIG. 14: Effect of mAbs on aggregation half-times in seeded a-syn aggregation. (A) Change in aggregation half-time (τ½), relative to the control in the absence of mAb. An antibody not binding to a-syn was used as isotype control (B) Percent increase of τ½ values, relative to the control in the absence of mAb, is plotted for the seeded aggregation in the presence of the indicated mAbs. Error bars represent the propagation of error (Equation 5). Significance was determined using a one-way ANOVA (Dunnett's multiple comparisons test) versus aggregation in the absence of mAb (****) P<0.0001).

FIG. 15: Effect of biparatopic antibodies (bAbs) on aggregation half-times in seeded a-syn aggregation. (A) Change in τ½ values, relative to no mAb control, from in vitro alpha-synuclein aggregations in the presence of the indicated bAbs. Error bars represent calculated SEM. (B) Percent increases of τ½ values, relative to the absence of antibody, are plotted for the seeded aggregations in the presence of the indicated bAb. Error bars represent the propagation of error (Equation 5).

FIG. 16: Inhibition of alpha-synuclein seeding capacity and aggregation in a cellular model. Percentage of de novo alpha-synuclein aggregates formed, relative to conditions in the presence of isotype control Ab. Error bars represent the propagation of error. Significance was determined using a one-way ANOVA (Uncorrected Fisher's LSD test) versus aggregation with isotype control Ab ((*) P<0.033; (**) P<0.002).

FIG. 17: Inhibition of alpha-synuclein seeding capacity and aggregation in a cellular model. Percentage of de novo alpha-synuclein aggregates formed, relative to conditions in the absence of antibody, as a function of antibody concentration. Dose-response curves were plotted and IC50 values of 18.0 nM (ACI-3112H1_1101C8), 21.6 nM (ACI-4301D5_3108C10), 8.6 nM (ACI-1108B11_27D8), 22.7 nM (ACI-5A12_3108C10), and 15.2 nM (ACI-4F3_5A12) were obtained using Equation 8.

### EXAMPLES

### Preparation of an Alpha-Synuclein Liposomal Vaccine Composition

The liposome-based antigenic constructs were prepared according to the protocols published in WO2012/055933. The liposomal vaccine with human full-length alpha-synuclein protein as antigen was used for antibody generation (Table 2, SEQ ID NO: 1) or liposomal vaccine with alpha-synuclein peptide as antigen was used for antibody generation.

TABLE 2
antigen description

| | | Amino acid sequence |
|---|---|---|
| | Definition | (1-letter code) |
| SEQ ID | FL-alpha- | MDVFMKGLSKAKEGVVAAAEKTKQG |
| NO: 1 | synuclein | VAEAAGKTKEGVLYVGSKTKEGVVH |
| | (140 aa) | GVATVAEKTKEQVTNVGGAVVTGVT |
| | | AVAQKTVEGAGSIAAATGFVKKDQL |
| | | GKNEEGAPQEGILEDMPVDPDNEAY |
| | | EMPSEEGYQDYEPEA |

### Mouse Immunization

Female C57BL/6JOIaHsd and BALB/cOIaHsd mice (Envigo, USA) were vaccinated at 10 weeks of age. C57BL/6JOIaHsd substrain is known to have a spontaneous deletion of the alpha-synuclein gene. Mice were vaccinated with vaccine containing human full-length alpha-synuclein protein or alpha-synuclein peptide presented on the surface of liposomes in the presence of synthetic monophosphoryl hexa-acyl Lipid A 3-deacyl (3D-(6-acyl) PHAD®) as adjuvant.

Mice were vaccinated by subcutaneous injection (s.c.) on days 0, 5, 8, 21, 35, 84, and in some cases on day 14, 28, 63 and 398. Mice were bled and heparinized plasma prepared 7 days before immunization (pre-immune plasma) and on days 14, 28, 40, 84, 90 and in some cases on day 7, 21, 35 and 308 after first immunization. Mice used for myeloma fusion were additionally vaccinated with three or four daily booster injections by intraperitoneal injection (i.p.) of liposomal vaccines without adjuvant. Very high antigen-specific IgG responses were obtained in all immunized mice.

### Isolation of Clonal Mouse Hybridoma Cell Lines Producing Specific and High-Affinity Monoclonal Antibodies

Mice were euthanized and fusion with PAI myeloma cells was performed using splenocytes from immunized mice. For screening fusion products, cell culture supernatant was diluted 1:50 and analysed using Luminex bead-based multiplex assay (Luminex, The Netherlands). Luminex beads were conjugated to either full-length alpha-synuclein, alpha-synuclein peptide 1-60aa, alpha-synuclein peptide 1-95aa, alpha-synuclein peptide 61-140aa, or full-length beta-synuclein (irrelevant target), and with capturing IgGs with anti-mouse IgG-Fc antibodies specific for the IgG1, IgG2a, IgG2b, IgG2c, and IgG3 subclasses (Jackson Immunoresearch, USA). Luminex assay results binding to full-length alpha-synuclein identified 92 hits. In a second round of fusion of immunized mice splenocytes and PAI myeloma cells, 400 hits were identified by Luminex assay binding to full-length alpha-synuclein. Viable hybridomas were grown using serum-containing selection media, and the best hybridomas binding to full-length alpha-synuclein were then selected for subcloning. Following limiting dilution, the clonal hybridomas were grown in low immunoglobulin containing medium and stable colonies were selected for antibody screening and selection.

In another round of fusion of immunized mice splenocytes or lymph nodes (popliteals, axial, brachials, and inguinals) and X63/AG.8653 myeloma cells, 279 hits were identified by ELISA assay binding to alpha-synuclein peptide 1-120aa (SEQ ID NO: 863). Viable hybridomas were grown using serum-containing selection media, and the best hybridomas binding to alpha-synuclein peptide were then selected for subcloning. Following limiting dilution, the clonal hybridomas were grown in low immunoglobulin containing medium and stable colonies were selected for antibody screening and selection.

### Isolation of Alpha Synuclein Antibodies by Phage Display

Some antibodies were also generated by phage display using the same group of immunized mice previously described. RT-PCR was performed on mRNA isolated from splenocytes. VH and VL region were assembled as scFv and cloned into phagemid vectors resulting in a phage display library of 1×10⁷ clones. Several rounds of panning were performed either against full length human alpha synuclein or against alpha synuclein fragments, 1-60 aa (SEQ ID NO: 850), 61-95 aa (SEQ ID NO: 851), or 96-140aa (SEQ ID NO: 147) (Table 3). Positive clones were sequenced and expressed recombinantly as murine IgG2a for characterization.

### Antibody Binding to Human Full-Length Alpha-Synuclein

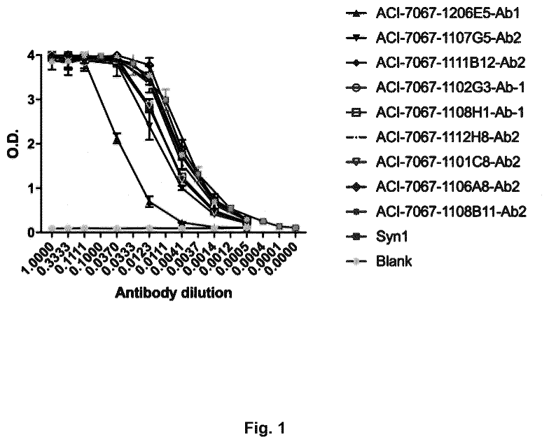

Antibody binding to human full-length alpha-synuclein was determined using an indirect ELISA. Full-length alpha-synuclein was diluted in carbonate/bicarbonate buffer pH 9.6 (Sigma, C3041) to a final concentration of 2.5pg/ml and coated onto ELISA plates overnight at 4° C. After washing with PBS/0.05% Polyethylene glycol sorbitan monolaurate (Tween®20) and blocking for 1 hour at 37° C. (PBS/0.05% Tween®20/1% BSA), plates were incubated for 2 hours at 37° C. with three-fold dilution series of alpha-synuclein antibodies from 1 μg/mL to 0.0005 μg/mL using PBS/0.05% Tween®20/1% BSA as diluent. Dilution series (three-fold from 0.1 μg/mL to 0.0001 μg/mL) of Syn1 antibody (BD Biosciences, 610787; epitope 91-99aa) was used as positive control, where applicable. Next, plates were washed with PBS/0.05% Tween® 20 and incubated for 2 hours at 37° C. with the detection antibody, anti-mouse IgG conjugated to alkaline phosphatase (Jackson Immunoresearch Laboratories Inc., 115-055-164,) at 1:1000 dilution. After final wash, plates were incubated 2 hours at 25° C. with 1 mg/mL of alkaline phosphatase substrate (p-nitrophenyl phosphate disodium hexahydrate; pNPP, S0942, Sigma) and read the absorbance optical density (O.D.) signal at 405 nm using an ELISA plate reader (Tecan, Switzerland). All generated antibodies show very good binding to human full-length alpha-synuclein (FIG. 1).

### Epitope Mapping on Alpha-Synuclein

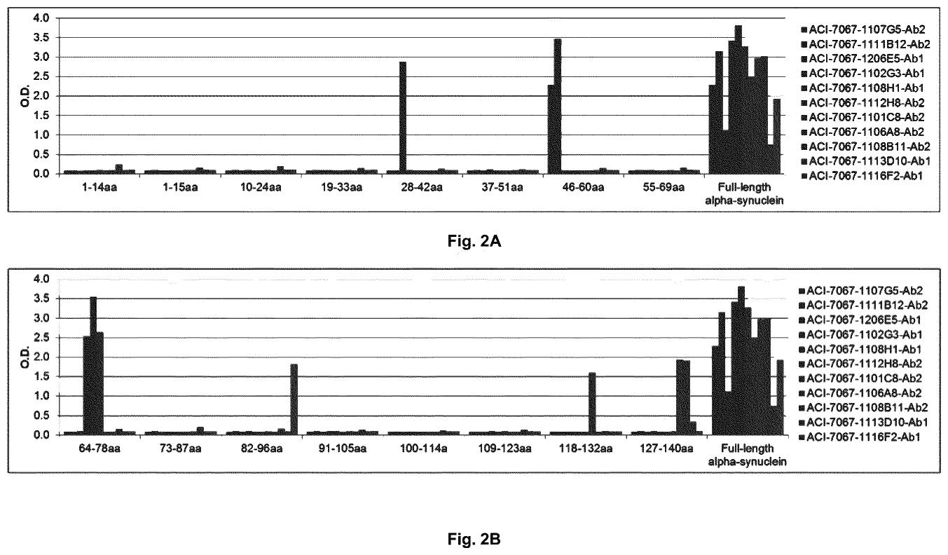

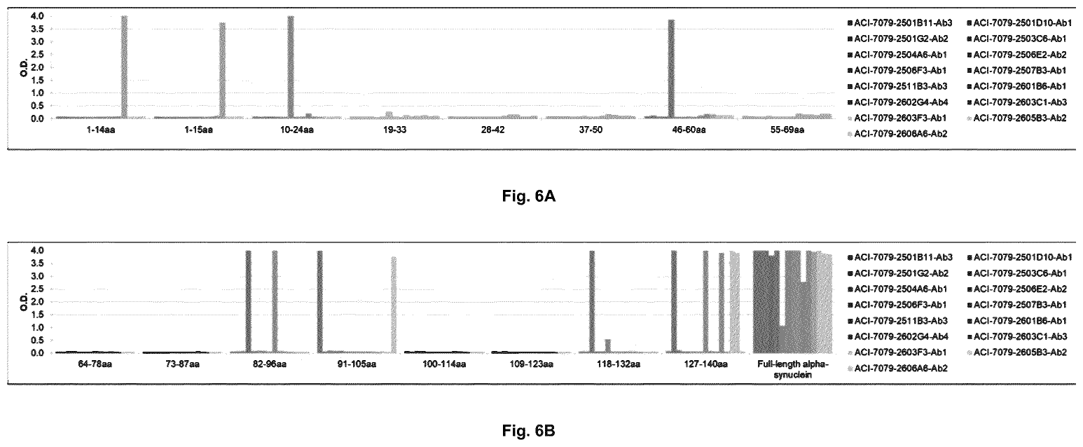

Serum-free supernatants were harvested from stable hybridomas. The supernatants containing antibodies of interest were then screened by an indirect ELISA assay to determine epitopes. Epitopes were first determined using a library of 15-mer peptides covering the entire sequence of human alpha-synuclein protein, spanning amino acids (aa) 1-140 with 9aa offset and Baa overlap. All peptides were synthesized biotinylated at N-terminus with aminohexanoic acid spacer except the N-terminal peptide 1-14aa (SEQ ID NO: 120) which was synthesized biotinylated at the C-terminus. Briefly, streptavidin-coated ELISA plates were blocked overnight at 4° C. (PBS/0.05% Tween®20/1% BSA) and then incubated for 1 hour at 25° C. with 0.25 μM of biotinylated full-length alpha-synuclein protein or biotinylated 15-mer peptides. Peptide sequences are provided in Table 3, which includes further longer peptides also used for epitope mapping in similar fashion. Plates were washed with PBS/0.05% Tween®20 and then incubated with the hybridoma supernatants at 1/100 dilution for 1 hour at 25° C. Next, plates were washed with PBS/0.05% Tween®20 and incubated for 1 hour at 25° C. with the detection antibody, anti-mouse IgG conjugated to alkaline phosphatase (Jackson Immunoresearch Laboratories Inc., 115-055-164,) at 1:1000 dilution. After final wash, plates were incubated 2 hours at 25° C. with alkaline phosphatase substrate (p-nitrophenyl phosphate disodium hexahydrate; pNPP, S0942, Sigma) and read the absorbance optical density (O.D.) signal at 405 nm using an ELISA plate reader (Tecan, Switzerland). Tested antibodies were found to bind to the either of the following peptides: 1-14aa, 1-15aa, 10-24aa, 28-42aa, 46-60aa, 64-78aa, 82-96aa, 91-105aa, 118-132aa, 127-140aa or 81-120aa. For antibody, ACI-7079-26016NAbM, no linear epitope could be identified, no binding was observed to peptides of 15-mer length while antibody bound to full-length alpha-synuclein. Results are shown in FIG. 2 and FIG. 6.

TABLE 3
Library of peptides used for epitope mapping

| SEQ | | aa alpha- |
|---|---|---|
| ID | | synuclein |
| NO: | Sequence | sequence |
| 120 | MDVFMKGLSKAKEG | 1-14* |
| 121 | MDVFMKGLSKAKEGV | 1-15 |
| 122 | KAKEGVVAAAEKTKQ | 10-24 |
| 123 | AEKTKQGVAEAAGKT | 19-33 |
| 124 | EAAGKTKEGVLYVGS | 28-42 |
| 125 | VLYVGSKTKEGVVHG | 37-51 |
| 126 | EGVVHGVATVAEKTK | 46-60 |
| 127 | VAEKTKEQVTNVGGA | 55-69 |
| 128 | TNVGGAVVTGVTAVA | 64-78 |
| 129 | GVTAVAQKTVEGAGS | 73-87 |
| 130 | VEGAGSIAAATGFVK | 82-96 |
| 131 | ATGFVKKDQLGKNEE | 91-105 |
| 132 | LGKNEEGAPQEGILE | 100-114 |
| 133 | QEGILEDMPVDPDNE | 109-123 |
| 134 | VDPDNEAYEMPSEEG | 118-132 |
| 135 | MPSEEGYQDYEPEA | 127-140 |
| 137 | TVEGAGSIAAATGFVKKDQL | 81-120 |
| | GKNEEGAPQEGILEDMPVDP | |
| 850 | MDVFMKGLSKAKEGVVAAAE | 1-60 |
| | KTKQGVAEAAGKTKEGVLYV | |
| | GSKTKEGVVHGVATVAEKTK | |
| 851 | EQVTNVGGAVVTGVTAVAQK | 61-95 |
| | TVEGAGSIAAATGFV | |
| 147 | KKDQLGKNEEGAPQEGILED | 96-140 |
| | MPVDPDNEAYEMPSEEGYQD | |
| | YEPEA | |
| 863 | MDVFMKGLSKAKEGVVAAAE | 1-120 |
| | KTKQGVAEAAGKTKEGVLYV | |
| | GSKTKEGVVHGVATVAEKTK | |
| | EQVTNVGGAVVTGVTAVAQK | |
| | TVEGAGSIAAATGFVKKDQL | |
| | GKNEEGAPQEGILEDMPVDP | |

*Peptide biotinylated at C-terminus

Epitopes were further determined using a library of 8-mer peptides covering the alpha-synuclein sequences previously identified by indirect ELISA on a library of 15-mer peptides. The 8-mer peptides were designed with 1aa offset and 7aa overlap. Finally, for determining the critical residues for antibody binding an alanine scanning library of peptides was utilized covering the alpha-synuclein sequences previously identified with the library of 15-mer peptides. The peptides of the alanine scanning library were from 15 to 30 residues in length and synthesized with an alanine residue in each position substituting the natural residue in the sequence (except when the natural residue is alanine). All peptides were synthesized biotinylated at N-terminus with aminohexanoic acid spacer. For the indirect ELISA, streptavidin-coated ELISA plates were blocked overnight at 4° C. (PBS/0.05% Tween®20/1% BSA) and then incubated for 1 hour at 25° C. with 0.25 μM of biotinylated biotinylated peptides. Plates were washed with PBS/0.05% Tween®20 and then incubated with the hybridoma supernatants at 1/100 dilution for 1 hour at 25° C. Next, plates were washed with PBS/0.05% Tween®20 and incubated for 1 hour at 25° C. with the detection antibody, anti-mouse IgG conjugated to alkaline phosphatase (Jackson Immunoresearch Laboratories Inc., 115-055-164,) at 1:1000 dilution. After final wash, plates were incubated 2 hours at 25° C. with alkaline phosphatase substrate (p-nitrophenyl phosphate disodium hexahydrate; pNPP, S0942, Sigma) and read the absorbance optical density (O.D.) signal at 405 nm using an ELISA plate reader (Tecan, Switzerland). The binding epitopes for the antibodies obtained from hybridoma supernatants are shown in Table 4A.

Furthermore, binding epitopes were confirmed using recombinantly produced antibodies. Variable domain sequences were cloned into mammalian cell expression vectors and transiently transfected into CHO cells. Antibodies were purified from cell culture supernatant by standard protein A purification and were buffer exchanged in 1×PBS, prior to being tested for binding. The binding epitopes for the recombinantly produced antibodies are shown in Table 4B. In the event of inconsistency between the results obtained using recombinant proteins and the results obtained from hybridoma supernatants the recombinant protein result is accepted (because there is some risk of contamination when diluting hybridoma supernatants). The binding epitopes for recombinantly produced antibodies are shown in Table 4B.

TABLE 4A
Antibody binding epitopes

| | | | Critical residues (aa) - |
|---|---|---|---|
| Antibody Code | Hybridoma Code | Epitope (aa) | Alanine scanning library |
| ACI-7067-1206E5-Ab1 | 1206E5D2 | 36-40 (SEQ | 36-40 |
| | | ID NO: 2) | |
| ACI-7067-1107G5-Ab2 | 1107G5B6 | 51-57 (SEQ | 51-52, 55-57 |
| | | ID NO: 3) | |
| ACI-7067-1111B12-Ab2 | 1111B12H10 | 51-57 (SEQ | 51-57 |
| | | ID NO: 3) | |
| ACI-7067-1108H1-Ab1 | 1108H1E1 | 65-74 (SEQ | 65, 68-70, 73-74 |
| | | ID NO: 4) | |
| ACI-7067-1112H8-Ab2 | 1112H8C12 | 65-74 (SEQ | 65, 68-71, 73-74 |
| | | ID NO: 4) | |
| ACI-7067-1102G3-Ab1 | 1102G3F2 | 65-81 (SEQ | 65, 68-70, 73-81 |
| | | ID NO: 5) | |
| ACI-7067-1116F2-Ab1 | 1116F2A2 | 93-95 | 93-95 |
| ACI-7067-1101C8-Ab2 | 1101C8F7 | 124-131 (SEQ | 126-127 |
| | | ID NO: 7) | |
| ACI-7067-1113D10-Ab1 | 1113D10E3D5 | 128-135 (SEQ | 128, 133, 135 |
| | | ID NO: 8) | |
| ACI-7067-1106A8-Ab2 | 1106A8H3 | 131-140 (SEQ | 135-136 |
| | | ID NO: 9) | |
| ACI-7067-1108B11-Ab2 | 1108B11D3 | 131-140 (SEQ | 135-136 |
| | | ID NO: 9) | |
| ACI-7079-2603C1-Ab3 | 2603C1H6 | 1-15 (SEQ | 14 |
| | | ID NO: 121) | |
| ACI-7079-2506F3-Ab1 | 2506F3E12 | 10-24 (SEQ | 14 |
| | | ID NO: 122) | |
| ACI-7079-2504A6-Ab1 | 2504A6C8 | 51-58 (SEQ | 51-54, 57-58 |
| | | ID NO: 136) | |
| ACI-7079-2503C6-Ab1 | 2503C6H9 | 82-96 (SEQ | 92-94, 96 |
| | | ID NO: 130) | |
| ACI-7079-2511B3-Ab3 | 2511B3B12 | 82-96 (SEQ | 92-94, 96 |
| | | ID NO: 130) | |
| ACI-7079-2501B11-Ab3 | 2501B11C7 | 91-105 (SEQ | 98, 102 |
| | | ID NO: 131) | |
| ACI-7079-2606A6-Ab2 | 2606A6D5 | 91-105 (SEQ | 96, 98, 100, 102 |
| | | ID NO: 131) | |
| ACI-7079-2501G2-Ab2 | 2501G2E5 | 118-132 (SEQ | 127-128 |
| | | ID NO: 134) | |
| ACI-7079-2506E2-Ab2 | 2506E2G4 | 118-132 (SEQ | 118-132 |
| | | ID NO: 134) | |
| ACI-7079-2507B3-Ab1 | 2507B3G8 | 127-140 (SEQ | 129, 135 |
| | | ID NO: 135) | |
| ACI-7079-2602G4-Ab4 | 2602G4H1 | 127-140 (SEQ | 129, 135 |
| | | ID NO: 135) | |
| ACI-7079-2603F3-Ab1 | 2603F3H3 | 127-140 (SEQ | 129, 135-136 |
| | | ID No: 135) | |
| ACI-7079-2605B3-Ab2 | 2605B3D1 | 127-140 (SEQ | 129, 135-136 |
| | | ID NO: 135) | |
| ACI-7079-2601B6-Ab1 | 2601B6D2 | Non-linear | Non-linear epitope |
| | | epitope | |
| ACI-7087-4119E10-Ab2 | 4119E10D12 | 28-42(SEQ | 33-37 |
| | | ID NO: 124)/ | |
| | | 37-51(SEQ | |
| | | ID NO: 125) | |
| ACI-7087-4125E6-Ab1 | 4125E6D5 | Non-linear | Non-linear epitope |
| | | epitope | |
| ACI-7088-4301D5-Ab2 | 4301D5B10 | 28-42(SEQ | 37-42 |
| | | ID NO: 124)/ | |
| | | 37-51(SEQ | |
| | | ID NO: 125) | |
| ACI-7088-4301E12-Ab2 | 4301E12B9 | 82-96 (SEQ | 92-96 |
| | | ID NO: 130) | |
| ACI-7088-4301H3-Ab2 | 4301H3A5 | 91-105 (SEQ | 101 |
| | | ID NO: 131) | |
| ACI-7088-4303A1-Ab1 | 4303A1E7 | 1-15 (SEQ | 7-10 |
| | | ID NO: 121) | |
| ACI-7088-4303A3-Ab1 | 4303A3E4 | 37-51 (SEQ | n.d. |
| | | ID NO: 125) | |
| ACI-7088-4303B6-Ab1 | 4303B6C11 | 1-15 (SEQ | 7-10 |
| | | ID NO: 121) | |
| ACI-7088-4303H6-Ab1 | 4303H6D7 | 91-105 (SEQ | 99 |
| | | ID NO: 131) | |
| ACI-7088-4305H7-Ab1 | 4305H7A4 | 1-15 (SEQ | 7-10/101 |
| | | ID NO: 121)/ | |
| | | 91-105 (SEQ | |
| | | ID NO: 131) | |
| ACI-7088-4317A4-Ab1 | 4317A4D2 | 1-15 (SEQ | 7-10 |
| | | ID NO: 121) | |
| ACI-7089-4409F1-Ab1 | 4409F1A8 | 82-96 (SEQ | 92-96 |
| | | ID NO: 130) | |
| ACI-7089-4415G5-Ab1 | 4415G5A11 | Non-linear | Non-linear epitope |
| | | epitope | |
| ACI-7089-4417G6-Ab1 | 4417G6B12 | 37-51 (SEQ | N.D. |
| | | ID NO: 125) | |
| ACI-7089-4418C5-Ab1 | 4418C5G1 | 82-96 (SEQ | 92-96 |
| | | ID NO: 130) | |
| ACI-7089-4418F6-Ab1 | 4418F6G7 | 82-96 (SEQ | 92-96 |
| | | ID NO: 130) | |
| ACI-8033-5A12-Ab1 | 917.5A12A11C9 | 100-114(SEQ | 100-105 |
| | | ID NO: 132) | |
| ACI-8033-25A3-Ab1 | 917.25A3E9F6 | 81-120 (SEQ | 105-120 |
| | | ID NO: 137) | |
| ACI-8033-1G10-Ab1 | 917.1G10A10F6 | 109-123 (SEQ | 114-115 |
| | | ID NO: 133) | |
| ACI-8033-19A2-Ab1 | 917.19A2E9E5 | 109-123 (SEQ | 105-120 |
| | | ID NO: 133) | |
| ACI-8033-8C10-Ab1 | 917.8C10C6G3 | 82-96 (SEQ | 93-94 |
| | | ID NO: 130) | |
| ACI-8033-7A2-Ab1 | 917.7A2B6A9 | 109-123 (SEQ | 105-120 |
| | | ID NO: 133) | |
| ACI-8033-1A12-Ab1 | 917.1A12C1B4 | 109-123 (SEQ | 112-114 |
| | | ID NO: 133) | |
| ACI-8033-4F3-Ab1 | 917.4F3F4G6 | 91-105 (SEQ | 99 |
| | | ID NO: 131) | |
| ACI-8033-17F5-Ab1* | 917.17F5F5G9 | 91-105 (SEQ | 91-105* |
| | | ID NO: 131)* | |
| ACI-8033-18C11-Ab1 | 917.18C11A11F10 | 100-114 (SEQ | 100-105/108-113 |
| | | ID NO: 132) | |
| ACI-8033-18D12-Ab1 | 917.18D12F10D6 | 100-114 (SEQ | 100-105/108-113 |
| | | ID NO: 132) | |
| ACI-8033-1F8-Ab1 | 917.1F8D8E4 | 82-96 (SEQ | 92-96 |
| | | ID NO: 130) | |
| ACI-8033-22E5-Ab1 | 917.22E5C5F7 | 109-123 (SEQ | 115 |
| | | ID NO: 133) | |
| ACI-8033-27D8-Ab1 | 917.27D8E1H10E10 | 81-120 (SEQ | 105-120 |
| | | ID NO: 137) | |
| ACI-8033-21C8-Ab1 | 917.21C8E4C8 | 100-114 (SEQ | 100-105/108-113 |
| | | ID NO: 132) | |
| ACI-7067-4813-R4A-G7- | 4813-R4A-G7 | 96-140 (SEQ | N.D. |
| rec1 | | ID NO: 147) | |
| ACI-7079-3101E3-Ab1 | 3101E3C9 | 100-114 (SEQ | 108-114 |
| | | ID NO: 132) | |
| ACI-7079-3103D9-Ab1 | 3103D9C9 | 28-50 (SEQ | 33-37 |
| | | ID NO: 139) | |
| ACI-7079-3103G12-Ab2 | 3103G12D2B10 | 19-33 (SEQ | N.D. |
| | | ID NO: 123) | |
| ACI-7079-3104F12-Ab2 | 3104F12F2H7 | 1-15 (SEQ | 33-37 |
| | | ID NO: 121), | |
| | | 28-50 (SEQ | |
| | | ID NO: 139) | |
| ACI-7079-3106C5-Ab1 | 3106C5B7 | 28-50 (SEQ | 33-37 |
| | | ID NO: 139) | |
| ACI-7079-3106F2-Ab1 | 3106F2C8 | 1-15 (SEQ | N.D. |
| | | ID NO: 121) | |
| ACI-7079-3107E6-Ab1 | 3107E6A3 | 28-50 (SEQ | 33-37 |
| | | ID NO: 139) | |
| ACI-7079-3108C10-Ab2 | 3108C10G6 | 28-50 (SEQ | 33-37 |
| | | ID NO: 139) | |
| ACI-7079-3112H1-Ab1 | 3112H1F3 | 91-105 (SEQ | 99-101 |
| | | ID NO: 131) | |
| ACI-8031-6207G10-Ab1 | 6207G10E6 | 15-45 (SEQ | 30-40 |
| | | ID NO: 138) | |
| ACI-8032-6301A10-Ab2 | 6301A10E12 | 31-60 (SEQ | 33-39 |
| | | ID NO: 146) | |
| ACI-8032-6301C8-Ab2 | 6301C8G12 | 31-60 (SEQ | 33-39 |
| | | ID NO: 146) | |
| ACI-8032-6304F3-Ab1 | 6304F3D12 | 31-60 (SEQ | 33-39 |
| | | ID NO: 146) | |
| ACI-8032-6307F1-Ab2 | 6307F1H10 | 31-60 (SEQ | N.D. |
| | | ID NO: 146) | |
| ACI-8032-6314A3-Ab3 | 6314A3E4 | 31-60 (SEQ | 33-39 |
| | | ID NO: 146) | |
| ACI-8033-6401F2-Ab1 | 6401F2E3 | 81-120 (SEQ | 108-113 |
| | | ID NO: 137) | |
| ACI-8033-6402E2-Ab2 | 6402E2E4 | 81-120 (SEQ | 99-103 |
| | | ID NO: 137) | |
| ACI-8033-6402E10-Ab1 | 6402E10B3 | 81-120 (SEQ | 92-96 |
| | | ID NO: 137) | |
| ACI-8033-6403A4-Ab1 | 6403A4A3 | 81-120 (SEQ | 116-120 |
| | | ID NO: 137) | |
| ACI-8033-6403E11-Ab2 | 6403E11B4 | 81-120 (SEQ | 92-96 |
| | | ID NO: 137) | |
| ACI-8032-6301G2-Ab2 | 6301G2D11 | N.D. | N.D. |
| ACI-8032-6313G2-Ab1 | 6313G2A10 | N.D. | N.D. |
| ACI-8030-6106F5-Ab1 | 6106F5B10 | N.D. | N.D. |

N.D.: Not determined

TABLE 4B
Recombinantly produced antibody binding epitopes

| | | | Critical residues (aa) - |
|---|---|---|---|
| Antibody Code | Hybridoma Code | Epitope (aa) | Alanine scanning library |
| ACI-7087-4119E10-Ab2 | 4119E10D12 | 28-42(SEQ | 33-37 |
| | | ID NO: 124) | |
| ACI-7088-4305H7-Ab1 | 4305H7A4 | 1-15 (SEQ | 7-10 |
| | | ID NO: 121) | |
| ACI-8033-5A12-Ab1 | 917.5A12A11C9 | 100-114(SEQ | 100-105 |
| | | ID NO: 132) | |
| ACI-8033-17F5-Ab1 | 917.17F5F5G9 | 91-105 (SEQ | 91-105 |
| | | ID NO: 131) | |
| ACI-7079-3104F12-Ab2 | 3104F12F2H7 | 28-50 (SEQ | 33-37 |
| | | ID NO: 139) | |
| ACI-7079-3108C10-Ab2 | 3108C10G6 | 100-114 (SEQ | N.D. |
| | | ID NO: 132) | |

N.D.: Not determined, although it was determined that residues 100-105 are not sufficient to define the critical residues and thus the critical residues differ from those determined for ACI-8033-5A12-Ab1

### Inhibition or Delay of Seeded Alpha-Synuclein Aggregation

Monoclonal anti-alpha-synuclein antibodies were evaluated for their ability to inhibit the aggregation of alpha-synuclein in vitro. The presence of alpha-synuclein pre-formed aggregates (seeds) increases the de novo aggregation propensity of monomeric a-synuclein. Alpha-synuclein antibodies were incubated with alpha-synuclein seeds prior to adding the monomeric alpha-synuclein for the aggregation assay. Kinetics of alpha-synuclein aggregation were monitored by thioflavin T (ThT) fluorescence. The ability of alpha-synuclein antibodies to inhibit the seeded aggregation was quantified by a percent change in the aggregation half-time (time to reach half-maximum ThT fluorescence signal).

Alpha-synuclein recombinant protein (rPeptide, S-1001-4) at concentration of 5 mg/mL was re-suspended and dialyzed against DPBS (Slide-A-Lyzer Mini Dialysis 10K MWCO, ThermoScientific, 88404) four times of 60 minutes each at 4° C. Higher molecular weight species were then removed by centrifugal filtration (Microcon DNA Fast Flow Centrifugal Filter Unit with Ultracel membrane, Sigma, MRCFOR100). Sonicated alpha-synuclein fibrils were diluted with PBS to a final concentration of 1.0 mg/mL. Aggregations were assembled in a low-binding 96-well plates (ThermoScientific, 278752), in triplicate for each condition. Alpha-synuclein seeds were used at 1% the final concentration of monomeric alpha-synuclein (14 μM).

Alpha-synuclein seeds (34.5 pmoles) were incubated with alpha-synuclein antibodies (787 pmoles, ˜22.8 equivalents) for 1 hour at at 25° C. As a reference control, alpha-synuclein seeds were incubated without the addition of alpha-synuclein antibodies. The Syn303 antibody (BioLegend, 824301) was used as a reference standard (Tran et al., Cell Rep. 2014, 7(6):2054-65). To control for any non-alpha-synuclein specific effect from the antibodies, the mouse IgG2a isotype control (IgG2a) (ThermoFisher, 02-6200) was used as a negative control.

Monomeric aSyn and ThT (3 mM stock solution, Sigma, D8537) were added to reach a final concentration of 14 μM and 46 μM respectively. Each aggregation was then aliquoted into 3 separate wells (65 μL/well) of the 96-well plates. Kinetic measurements were performed using an M200 Infinite Pro Microplate Reader (Tecan, Switzerland).

ThT fluorescent measurements were obtained in triplicate for each aggregation condition (technical repeats) and run twice on independent days (for a total of N=6). A baseline correction was performed by subtraction of the initial ThT value (t=0) and data was then normalized as a percent maximum ThT signal (see Equation 1). Aggregation half-times (τ½) were calculated from non-linear regressions using either a sigmoidal dose-response (see Equation 2) or a one-phase association (see Equation 3) (GraphPad Prism 7) and represent the time taken to reach half the maximum ThT signal.

$$\% ThT ( x ) = \frac{( ThT ( x ) ) - ( ThT ( {x}_{0} ) )}{( ThT ( {x}_{max} ) ) - ( ThT ( {x}_{0} ) )} * 100 \qquad Equation 1$$

Where % ThT(x) is the percent ThT signal at time t=x, ThT(x₀) is the ThT signal at t=0 and ThT(xₘₐₓ) is the maximum ThT signal.

$$\% ThT ( x ) = Bottom + \frac{( Top - Bottom )}{( 1 + {10}^{( {LogEC}_{50} - X ) - HillSlope} )} \qquad Equation 2$$

Where Bottom is a fit of the minimum ThT signal, Top is a fit of the maximum ThT signal, EC50 is the x value when the ThT signal is halfway between Bottom and Top, and the HillSlope is the steepness of the curve. Here, the aggregation half-time (τ_{1/2}) is obtained directly from EC50.

$$\% ThT ( x ) = ThT ( {x}_{0} ) + ( ( Plateau - ThT ( {x}_{0} ) ) * ( 1 - exp ( - K * x ) ) \qquad Equation 3$$

Where ThT(x₀) is the initial ThT signal, Plateau is the fit of the maximum ThT signal, and K is the rate constant. Here, the aggregation half-time (τ_{1/2}) is calculated from ln(2)/K.

$$\% Increase {τ}_{1 / 2} = \frac{{τ}_{mAb} - {τ}_{no mAb}}{{τ}_{no mAb}} * 100 \qquad Equation 4$$

Where τ_{no mab} is the aggregation half-time in the absence of antibody (mAb) and τ_{mab} is the aggregation half-time in the presence of the indicated antibody.

$$Propagation of Error = {τ}_{mAb} * \sqrt{{( \frac{{SEM}_{mAb}}{{τ}_{mAb}} )}^{2} + {( \frac{{SEM}_{no mAb}}{{τ}_{no mAb}} )}^{2}} \qquad Equation 5$$

Where τ_{no mab} is the aggregation half-time in the absence of mAb, τ_{mab} is the aggregation half-time in the presence of the indicated mAb, and SEM is the standard error (calculations resulting from fitting of Equations 2 and 3).

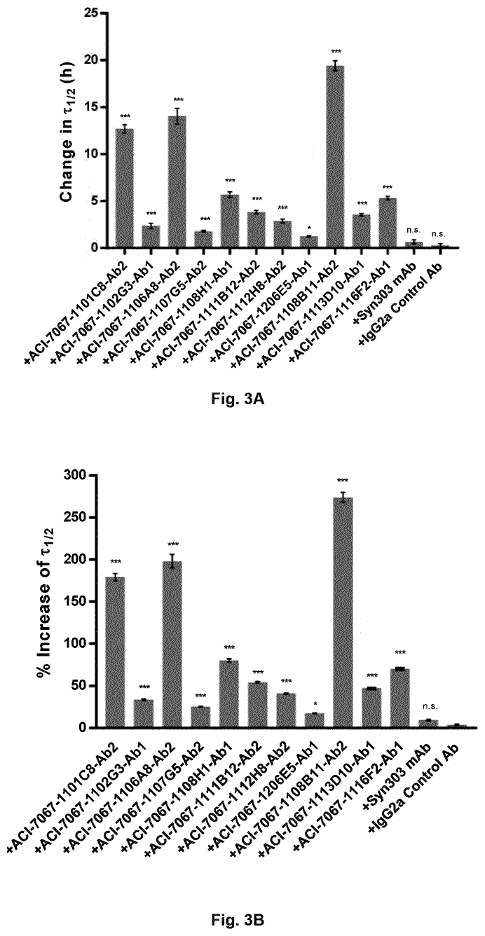

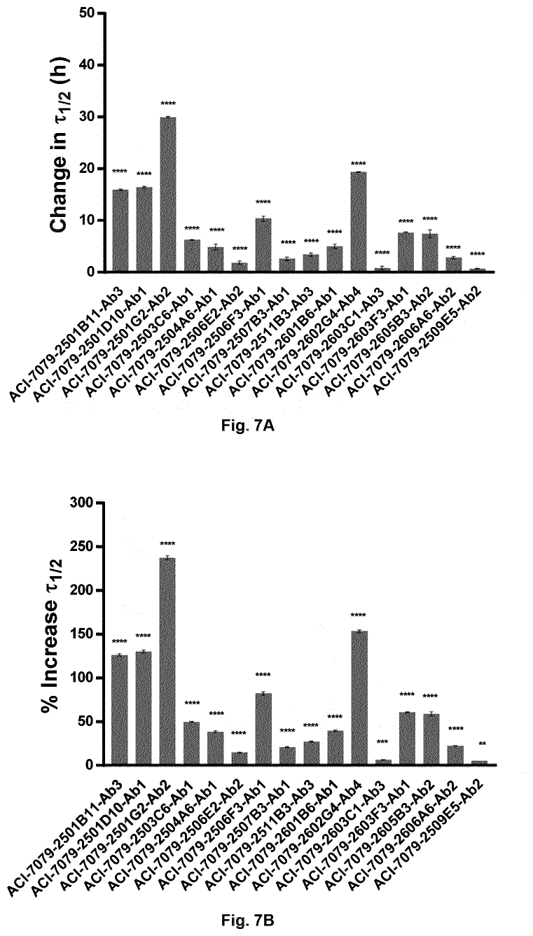

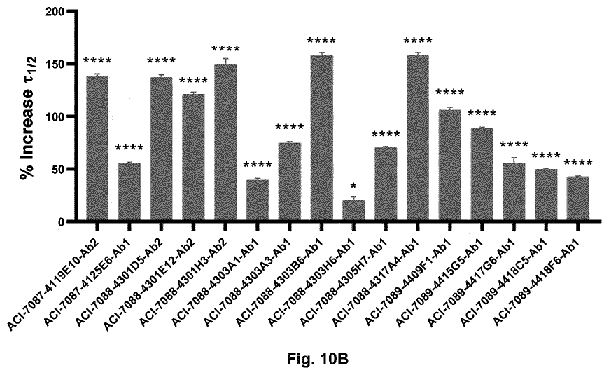

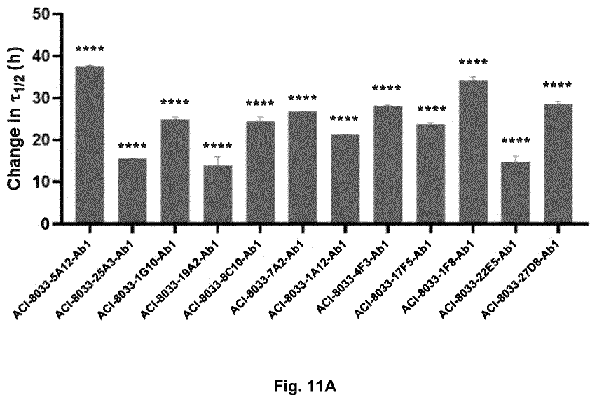

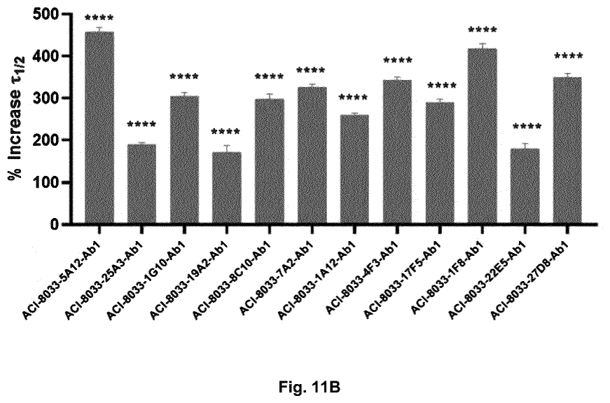

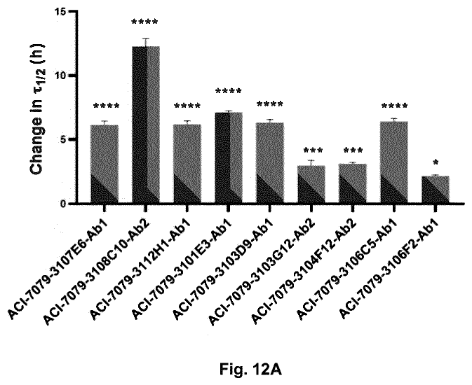

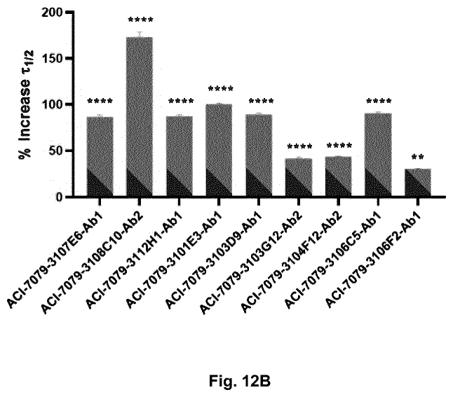

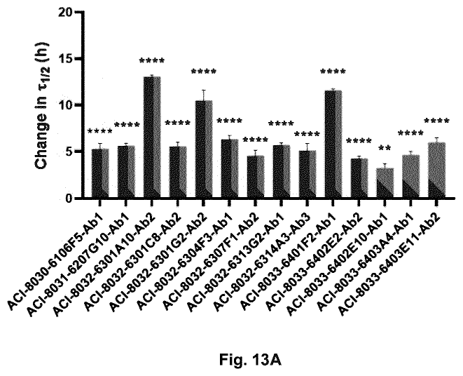

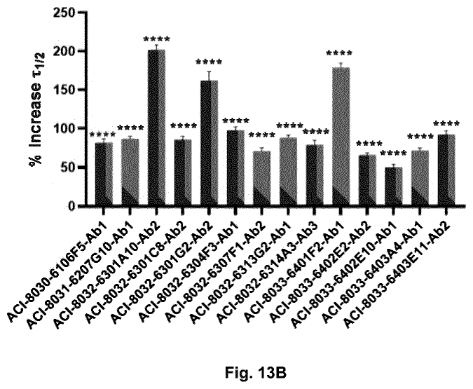

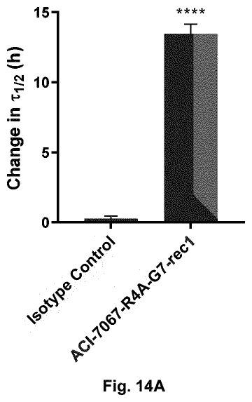

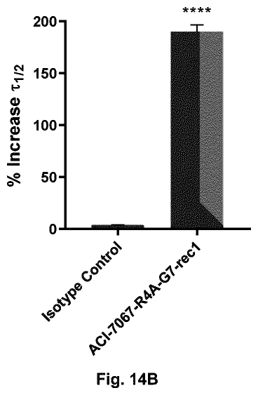

Aggregation half-times (τ_{1/2}) were obtained using either a sigmoidal fit (Equation 2) or an exponential fit (Equation 3) dependent upon the kinetic profile and best fit. Varied time frames were used to obtain optimal fitting as ThT signals can decrease following completion of aggregation. Change in τ_{1/2} values, in the presence of the indicated antibodies, were normalized relative to the τ_{1/2} value in the absence of antibody. FIG. 3A, FIG. 7A, FIG. 10A, FIG. 11A, FIG. 12A, FIG. 13A and FIG. 14A show the comparison of changes in τ_{1/2} values as normalized to the aggregation in the absence of antibody. Significant increases in τ_{1/2} values were observed for all antibodies proving the good efficacy of antibodies in delaying the seeded and/or spontaneous aggregation of alpha-synuclein. Pre-incubation with either Syn303 or the IgG2a control showed no significant effect on the seeded aggregation (FIG. 3A).

The percent increase in τ_{1/2} values were calculated relative to the seeded aggregation in the absence of antibody (see Equation 4). FIG. 3B, FIG. 7B, FIG. 10B, FIG. 11B, FIG. 12B, FIG. 13B and FIG. 14B show the calculated percent increase in τ_{1/2} values upon pre-incubation of alpha-synuclein seeds with the indicated antibodies proving the good efficacy of antibodies in delaying the seeded and/or spontaneous aggregation of alpha-synuclein. Relative to the IgG2a control, no significant change increase in τ_{1/2} was observed for pre-incubation with the commercially available Syn303 antibody (FIG. 3B). Pre-incubation of alpha-synuclein seeds with all antibodies of the present invention showed a significant percent increase in τ_{1/2} values.

ACI-8032-6301A10-Ab2 demonstrated the largest increase in τ_{1/2} values, closely followed by ACI-8033-6401F2-Ab1, ACI-7079-3108C10-Ab2 and ACI-8032-6301G2-Ab2. Similar results were obtained with ACI-7067-4813-R4A-G7-rec1 (FIG. 14). Relative to the control condition, aggregation in the absence of antibody, pre-incubation of alpha-synuclein seeds with all antibodies of the present invention showed a significant percent increase in τ_{1/2} values.

Affinity measurements on alpha-synuclein monomers and alpha-synuclein fibrils by SPR

Affinity measurements were performed on an surface plasmon resonance (SPR) instrument (Biacore T200, GE Healthcare Life Sciences) using CM5 Series S sensor chips (GE Healthcare, BR-1005-30). Flow channels (Fc) 1-4 were activated with a fresh solution of EDC/NHS (Amine Coupling Kit, 1:1 ratio of both reagents, GE Healthcare, BR-1006-33). The goat anti-mouse antibody (GE Healthcare, BR-1008-38) was captured at a concentration of 30 μg/mL diluted in 10 mM sodium acetate (pH 5.0). Following, all unreacted activated ester groups were capped with 1 M ethanolamine (GE Healthcare, BR-1006-33). Any non-covalently bound antibodies were removed by three successive regenerations of 10 mM Glycine pH 1.7 (GE Healthcare, 28-9950-84). Immobilization levels were evaluated following ethanolamine capping (Bound) and finally following regeneration (Final). Non-covalent immobilization of alpha-synuclein antibodies was performed using a target immobilization method of 2000 response units (RU). Antibodies were diluted in 10 mM sodium acetate pH 5.5 (GE Healthcare, BR-1003-52) to a final concentration of 5 μg/mL.

Binding affinity of alpha-synuclein antibodies to monomeric or fibrillar alpha-synuclein species was performed using a single-cycle kinetics method. The instrument was primed with 1×HBS-P+ buffer (10× stock from GE Healthcare, BR-1003-52 diluted in Milli-Q water). Injections of monomeric alpha-synuclein (aSyn) (Boston Biochem, SP-485), increasing in concentration from 0.62-50 nM prepared from serial 2-fold dilutions, were performed with contact times of 300 sec/injection at a flow rate of 30 μL/min. A dissociation phase of 900 sec followed the final 50 nM injection. Regeneration of the sensor to the goat anti-mouse antibody layer was achieved using 3 regenerations of 10 mM Glycine pH 1.7. Injections of alpha-synuclein fibrils of increasing in concentration from 5.56-450 nM prepared from serial 2-fold dilutions, were performed with contact times of 300 sec/injection at a flow rate of 30 μL/min. A dissociation phase of 900 sec followed the final 450 nM injection. Regeneration of the sensor to the goat anti-mouse antibody layer was achieved using 3 regenerations of 10 mM Glycine pH 1.7. Results obtained from single-cycle kinetics were evaluated by Biacore T200 evaluation software with 1:1 binding homogenous Langmuir model (with a global Rmax) with Cycle 5 as a blank subtraction. The following kinetic parameters were obtained: on-rate (ka), off-rate (kd), affinity constant (KD, ratio of kd by ka), maximum response (Rmax), and goodness of fit (Chi2).

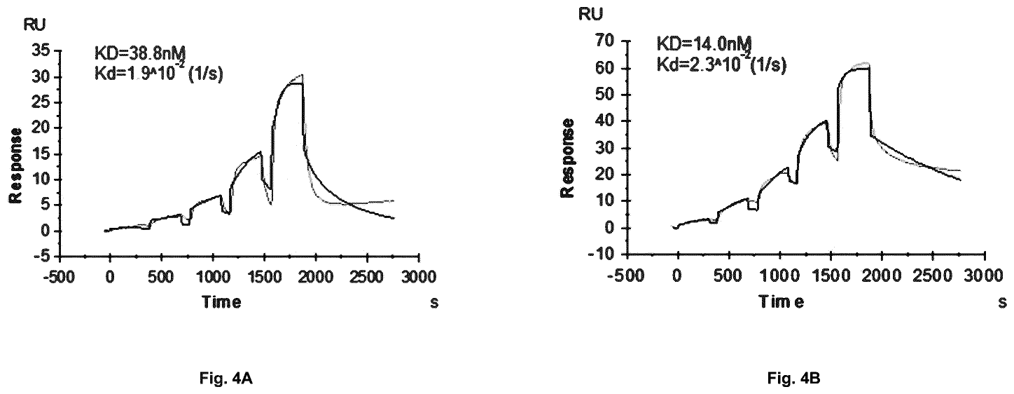

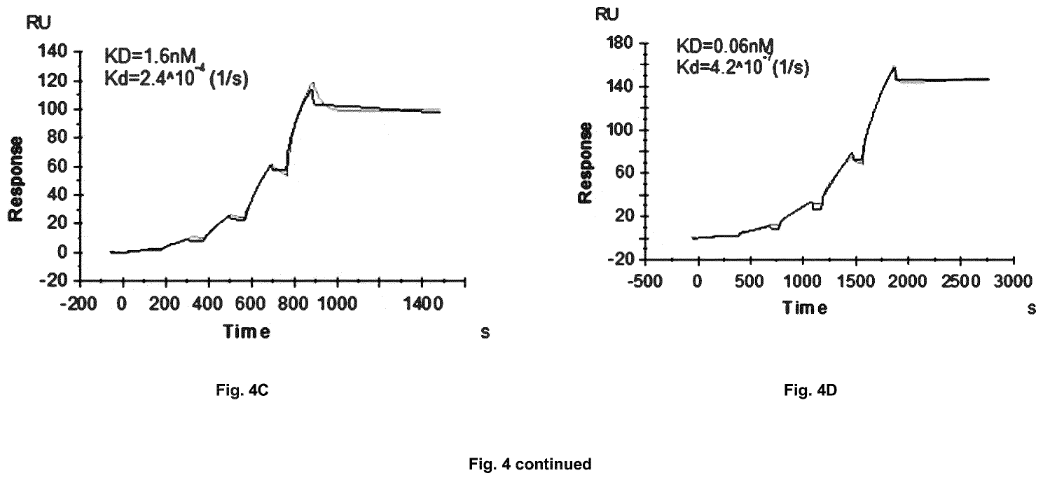

Non-covalent capture of the alpha-synuclein antibodies was performed in three separate runs. Capture levels ranged from ˜1800 to ˜2100 RU based on the target immobilization level of 2000 RU. Sensograms were obtained for responses to monomeric and fibrillar alpha-synuclein, representative examples for two antibodies are shown in FIG. 4. Kinetic constants were determined from 1:1 homogenous binding models for most of the cases. For ACI-7067-1101C8-Ab2 versus monomeric aSyn, a heterogeneous ligand model was used to obtain ka and kd values and steady-state model was used to determine KD and Rmax. The kinetic fitting parameters from single-cycle kinetics affinity measurements by SPR are shown in Table 5. ACI-7067-1101C8-Ab2, ACI-7079-2503C6-Ab1, ACI-7079-2603F3-Ab1, ACI-7088-43031B6-Ab1, ACI-8033-4F3-Ab1, ACI-7067-1113D10-Ab1, ACI-7067-4813-R4A-G7-rec1, ACI-7079-3101E3-Ab1, ACI-8032-6301A10-Ab2, ACI-7079-3106F2-Ab1, ACI-8033-6403A4-Ab1 demonstrate a binding preference to fibrillar alpha-synuclein and display significantly slower dissociation rates (Kd) from fibrillar alpha-synuclein compared to monomeric alpha-synuclein (FIG. 4). Moreover, ACI-7079-3108C10-Ab2 and ACI-8033-6401 F2-Ab1 selectively bind only to fibrillar alpha-synuclein.

TABLE 5
Affinity measurements obtained by SPR

| | | Alpha-synuclein monomers | | | Alpha-synuclein fibrils | | |
|---|---|---|---|---|---|---|---|
| Antibody Code | Hybridoma Code | ka (1/Ms) | kd (1/s) | KD (nM) | ka (1/Ms) | kd (1/s) | KD (nM) |
| ACI-7067-1101C8-Ab2 | 1101C8F7 | 5.55E+04 | 2.65E−02 | 43.7 | 1.76E+05 | 2.83E−04 | 2.9 |
| ACI-7067-1102G3-Ab1 | 1102G3F2 | 1.29E+05 | 1.03E−03 | 8 | 4.60E+04 | 1.35E−03 | 30.6 |
| ACI-7067-1106A8-Ab2 | 1106A8H3 | 2.18E+05 | 5.00E−03 | 23.2 | 2.84E+05 | 7.21E−03 | 25 |
| ACI-7067-1107G5-Ab2 | 1107G5B6 | 1.58E+05 | 1.14E−03 | 7.2 | 3.45E+05 | 2.18E−03 | 16.1 |
| ACI-7067-1108H1-Ab1 | 1108H1E1 | 1.31E+05 | 5.65E−04 | 4.4 | 2.71E+05 | 1.18E−03 | 14.3 |
| ACI-7067-1111B12-Ab2 | 1111B12H10 | 1.72E+05 | 1.40E−03 | 8.1 | 2.63E+04 | 1.01E−03 | 39.2 |
| ACI-7067-1112H8-Ab2 | 1112H8C12 | 2.50E+05 | 1.58E−03 | 6.3 | 2.85E+05 | 2.45E−03 | 18.7 |
| ACI-7067-1108B11-Ab2 | 1108B11D3 | 1.91E+05 | 1.54E−03 | 8.1 | 2.51E+05 | 2.05E−03 | 18.6 |
| ACI-7067-1113D10-Ab1 | 1113D10E3D5 | 1.03E+04 | 2.26E−02 | 14 | 6.94E+03 | 4.16E−07 | 0.06 |
| ACI-7067-1116F2-Ab1 | 1116F2A2 | 4.70E+04 | 2.32E−04 | 4.9 | 8.30E+03 | 4.58E−04 | 55.1 |
| ACI-7067-1206E5-Ab1 | 1206E5D2 | 1.65E+05 | 6.36E−05 | 0.4 | 5.73E+04 | 4.05E−04 | 8.3 |
| ACI-7079-2501B11-Ab3 | 2501B11C7 | 1.41E+05 | 3.35E−04 | 2.4 | 1.09E+04 | 3.47E−04 | 31.8 |
| ACI-7079-2501D10-Ab1 | 2501D10C3 | 2.64E+05 | 4.30E−04 | 1.6 | 1.73E+04 | 4.27E−04 | 24.7 |
| ACI-7079-2501G2-Ab2 | 2501G2E5 | 2.91E+05 | 8.40E−04 | 2.9 | 1.90E+04 | 5.28E−04 | 27.8 |
| ACI-7079-2503C6-Ab1 | 2503C6H9 | 4.05E+04 | 1.20E−04 | 3.0 | 9.45E+03 | 3.28E−07 | 0.004 |
| ACI-7079-2504A6-Ab1 | 2504A6C8 | 1.63E+05 | 2.36E−04 | 1.4 | 1.66E+04 | 1.92E−04 | 11.5 |
| ACI-7079-2506E2-Ab2 | 2506E2G4 | 8.09E+04 | 6.15E−04 | 7.6 | 5.19E+03 | 4.76E−04 | 91.9 |
| ACI-7079-2506F3-Ab1 | 2506F3E12 | 2.10E+05 | 5.10E−04 | 2.4 | 1.83E+04 | 1.61E−04 | 8.8 |
| ACI-7079-2507B3-Ab1 | 2507B3G8 | 2.45E+05 | 6.42E−04 | 2.6 | 1.91E+04 | 6.68E−04 | 34.9 |
| ACI-7079-2511B3-Ab3 | 2511B3B12 | 9.28E+04 | 5.83E−04 | 6.3 | 1.06E+04 | 3.12E−04 | 29.5 |
| ACI-7079-2601B6-Ab1 | 2601B6D2 | 2.90E+05 | 2.38E−02 | 82.1 | 1.74E+04 | 3.82E−04 | 22.0 |
| ACI-7079-2602G4-Ab4 | 2602G4H1 | 2.23E+05 | 8.67E−04 | 3.9 | 1.24E+04 | 2.34E−04 | 18.9 |
| ACI-7079-2603C1-Ab3 | 2603C1H6 | 5.58E+08 | 6.06E+00 | 10.9 | 1.28E+09 | 5.17E+01 | 40.3 |
| ACI-7079-2603F3-Ab1 | 2603F3H3 | 5.08E+04 | 1.49E−02 | 292.8 | 7.31E+03 | 1.60E−08 | 0.002 |
| ACI-7079-2605B3-Ab2 | 2605B3D1 | 2.83E+05 | 1.09E−03 | 3.9 | 1.68E+04 | 2.94E−04 | 17.4 |
| ACI-7079-2606A6-Ab2 | 2606A6D5 | 8.60E+05 | 4.12E−03 | 4.8 | 1.54E+04 | 8.62E−04 | 56.2 |
| ACI-7087-4119E10-Ab2 | 4119E10D12 | 1.80E+04 | 1.88E−02 | 1042.5 | 2.80E+05 | 4.53E−03 | 16.2 |
| ACI-7087-4125E6-Ab1 | 4125E6D5 | 5.91E+04 | 2.61E−02 | 442.1 | 1.12E+04 | 5.67E−04 | 50.7 |
| ACI-7088-4301D5-Ab2 | 4301D5B10 | 5.69E+04 | 3.53E−02 | 619.8 | 1.08E+04 | 3.85E−04 | 35.8 |
| ACI-7088-4301E12-Ab2 | 4301E12B9 | 1.98E+04 | 1.18E−04 | 6.0 | 4.25E+03 | 1.66E−04 | 39.0 |
| ACI-7088-4301H3-Ab2 | 4301H3A5 | 2.76E+04 | 3.29E−03 | 119.0 | 1.04E+04 | 8.98E−04 | 86.5 |
| ACI-7088-4303A1-Ab1 | 4303A1E7 | 1.70E+06 | 6.32E−02 | 37.1 | 1.81E+04 | 2.44E−04 | 13.5 |
| ACI-7088-4303A3-Ab1 | 4303A3E4 | 1.04E+06 | 1.07E−03 | 1.0 | 6.47E+04 | 6.06E−04 | 9.4 |
| ACI-7088-4303B6-Ab1 | 4303B6C11 | 1.35E+06 | 1.06E−01 | 78.5 | 1.37E+04 | 1.30E−04 | 9.5 |
| ACI-7088-4303H6-Ab1 | 4303H6D7 | 3.44E+04 | 6.70E−03 | 194.8 | 1.46E+04 | 6.36E−04 | 43.7 |
| ACI-7088-4305H7-Ab1 | 4305H7A4 | 2.73E+07 | 9.24E−02 | 3.4 | 2.42E+04 | 2.03E−04 | 8.4 |
| ACI-7088-4317A4-Ab1 | 4317A4D2 | 3.28E+03 | 1.20E−02 | 3655.6 | 3.37E+05 | 9.98E−04 | 3.0 |
| ACI-7089-4409F1-Ab1 | 4409F1A8 | 1.54E+05 | 9.92E−04 | 6.4 | 6.67E+05 | 5.87E−03 | 8.8 |
| ACI-7089-4415G5-Ab1 | 4415G5A11 | 6.00E+04 | 1.41E−04 | 2.4 | 2.42E+05 | 3.10E−04 | 1.3 |
| ACI-7089-4417G6-Ab1 | 4417G6B12 | 2.47E+08 | 5.58E+00 | 22.6 | 1.79E+05 | 8.74E−03 | 48.7 |
| ACI-7089-4418C5-Ab1 | 4418C5G1 | 4.50E+04 | 1.41E−04 | 3.1 | 2.07E+05 | 9.40E−06 | 0.05 |
| ACI-7089-4418F6-Ab1 | 4418F6G7 | 8.18E+04 | 9.25E−06 | 0.1 | 2.72E+05 | 1.14E−05 | 0.04 |
| ACI-8033-5A12-Ab1 | 917.5A12A11C9 | N.D. | N.D. | N.D. | 3.06E+04 | 4.37E−06 | 0.1 |
| ACI-8033-25A3-Ab1 | 917.25A3E9F6 | N.D. | N.D. | N.D. | N.D. | N.D. | N.D. |
| ACI-8033-1G10-Ab1 | 917.1G10A10F6 | N.D. | N.D. | N.D. | 1.77E+04 | 6.54E−05 | 3.7 |
| ACI-8033-19A2-Ab1 | 917.19A2E9E5 | 1.33E+07 | 8.28E−02 | 6.2 | 1.77E+04 | 1.19E−04 | 6.7 |
| ACI-8033-8C10-Ab1 | 917.8C10C6G3 | 4.31E+04 | 1.14E−04 | 2.6 | 4.96E+03 | 5.59E−05 | 11.3 |
| ACI-8033-7A2-Ab1 | 917.7A2B6A9 | N.D. | N.D. | N.D. | 8.81E+03 | 7.83E−05 | 8.9 |
| ACI-8033-1A12-Ab1 | 917.1A12C1B4 | 1.02E+06 | 3.27E−02 | 31.9 | 6.66E+03 | 6.53E−05 | 9.8 |
| ACI-8033-4F3-Ab1 | 917.4F3F4G6 | 9.70E+04 | 1.60E−04 | 1.7 | 4.50E+03 | 2.24E−07 | 0.05 |
| ACI-8033-17F5-Ab1 | 917.17F5F5G9 | 9.66E+04 | 2.32E−04 | 2.4 | 1.37E+04 | 1.20E−04 | 8.7 |
| ACI-8033-18C11-Ab1 | 917.18C11A11F10 | 1.43E+05 | 2.63E−04 | 1.8 | 8.27E+03 | 2.51E−04 | 30.4 |
| ACI-8033-18D12-Ab1 | 917.18D12F10D6 | 8.25E+07 | 2.60E−01 | 3.2 | 3.50E+02 | 2.33E−03 | 570.9 |
| ACI-8033-1F8-Ab1 | 917.1F8D8E4 | 3.04E+04 | 7.38E−04 | 24.3 | 9.83E+02 | 9.96E−04 | 1013.0 |
| ACI-8033-22E5-Ab1 | 917.22E5C5F7 | N.D. | N.D. | N.D. | 1.80E+04 | 3.27E−05 | 1.8 |
| ACI-8033-27D8-Ab1 | 917.27D8E1H10E10 | N.D. | N.D. | N.D. | N.D. | N.D. | N.D. |
| ACI-8033-21C8-Ab1 | 917.21C8E4C8 | 3.01E+05 | 4.82E−04 | 1.6 | 8.81E+03 | 1.56E−04 | 17.7 |
| ACI-7067-R4A-G7- | 4813-R4A-G7 | 7.35E+05 | 7.37E−02 | 97.0 | 4760.667 | 1.34E−07 | 0.041 |
| Ab-1 (also termed | | | | | | | |
| ACI-7067-4813-R4A- | | | | | | | |
| G7-rec1) | | | | | | | |
| ACI-7079-3101E3-Ab1 | 3101E3C9 | 1.90E+05 | 5.69E−02 | 300 | 9.38E+03 | 1.29E−04 | 13.7 |
| ACI-7079-3103D9-Ab1 | 3103D9C9 | 5.98E+05 | 4.37E−04 | 0.73 | 4.48E+04 | 4.13E−04 | 9.20 |
| ACI-7079-3103G12-Ab2 | 3103G12D2B10 | 5.80E+05 | 1.40E−03 | 2.40 | 2.50E+04 | 5.63E−04 | 22.5 |
| ACI-7079-3104F12-Ab2 | 3104F12F2H7 | 8.92E+05 | 4.52E−04 | 0.51 | 7.18E+04 | 2.68E−04 | 3.73 |
| ACI-7079-3106C5-Ab1 | 3106C5B7 | 7.92E+05 | 3.25E−03 | 4.10 | 2.50E+04 | 2.37E−04 | 9.49 |
| ACI-7079-3106F2-Ab1 | 3106F2C8 | 3.44E+05 | 1.35E−01 | 392 | 1.17E+04 | 3.67E−04 | 31.5 |
| ACI-7079-3107E6-Ab1 | 3107E6A3 | 7.65E+05 | 8.59E−04 | 1.12 | 5.51E+04 | 1.46E−04 | 2.66 |
| ACI-7079-3108C10-Ab2 | 3108C10G6 | Non-binding | Non-binding | Non-binding | 9.84E+03 | 6.66E−05 | 6.77 |
| ACI-7079-3112H1-Ab1 | 3112H1F3 | 2.14E+05 | 2.25E−03 | 10.5 | 1.12E+04 | 2.16E−04 | 19.3 |
| ACI-8030-6106F5-Ab1 | 6106F5B10 | 1.18E+05 | 2.30E−04 | 1.95 | 1.39E+04 | 2.32E−04 | 16.7 |
| ACI-8031-6207G10-Ab1 | 6207G10E6 | 8.53E+04 | 3.14E−04 | 3.67 | 1.13E+04 | 1.50E−04 | 13.3 |
| ACI-8032-6301A10-Ab2 | 6301A10E12 | 4.50E+05 | 5.17E−03 | 11.5 | 7.18E+03 | 1.93E−04 | 2.69 |
| ACI-8032-6301C8-Ab2 | 6301C8G12 | 3.76E+05 | 3.29E−03 | 8.76 | 8.30E+04 | 3.55E−03 | 42.8 |
| ACI-8032-6301G2-Ab2 | 6301G2D11 | 3.90E+02 | 2.37E−04 | 606 | 1.05E+02 | 3.53E−04 | 3350 |
| ACI-8032-6304F3-Ab1 | 6304F3D12 | 3.37E+06 | 1.60E−02 | 4.75 | 1.30E+04 | 5.27E−04 | 40.6 |
| ACI-8032-6307F1-Ab2 | 6307F1H10 | 1.30E+06 | 2.48E−02 | 19.0 | 2.65E+04 | 6.91E−04 | 26.0 |
| ACI-8032-6313G2-Ab1 | 6313G2A10 | 3.35E+04 | 5.41E−04 | 16.2 | 3.38E+02 | 8.29E−04 | 2460 |
| ACI-8032-6314A3-Ab3 | 6314A3E4 | 2.28E+06 | 3.82E−02 | 16.7 | 7.83E+03 | 3.25E−04 | 41.5 |
| ACI-8033-6401F2-Ab1 | 6401F2E3 | Non-binding | Non-binding | Non-binding | 1.31E+04 | 1.60E−04 | 12.2 |
| ACI-8033-6402E2-Ab2 | 6402E2E4 | 2.00E+05 | 6.51E−04 | 3.26 | 7.35E+03 | 9.87E−04 | 134 |
| ACI-8033-6402E10-Ab1 | 6402E10B3 | 2.05E+04 | 5.80E−04 | 28.3 | 4.06E+03 | 1.52E−04 | 37.4 |
| ACI-8033-6403A4-Ab1 | 6403A4A3 | 4.57E+05 | 4.40E−02 | 96.4 | 1.58E+04 | 5.99E−04 | 37.9 |
| ACI-8033-6403E11-Ab2 | 6403E11B4 | 7.52E+04 | 2.93E−04 | 3.90 | 6.11E+03 | 2.73E−04 | 44.6 |

N.D.: not determined

### Target Engagement on Human Alpha-Synuclein Aggregates

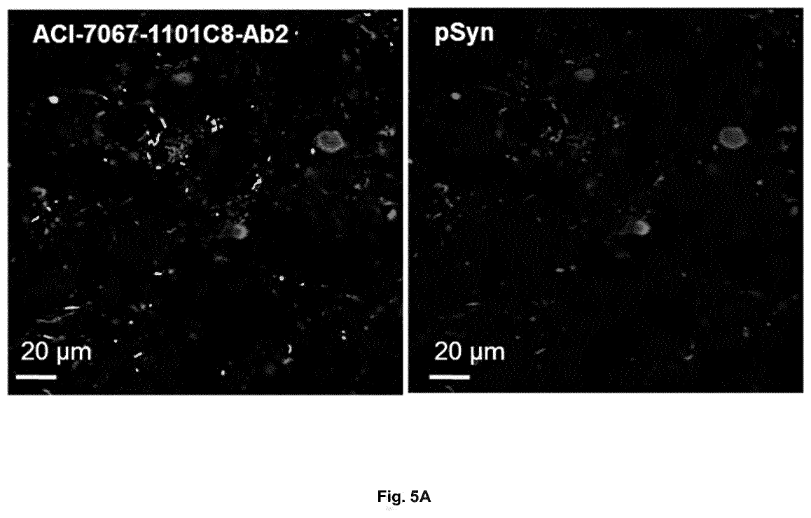

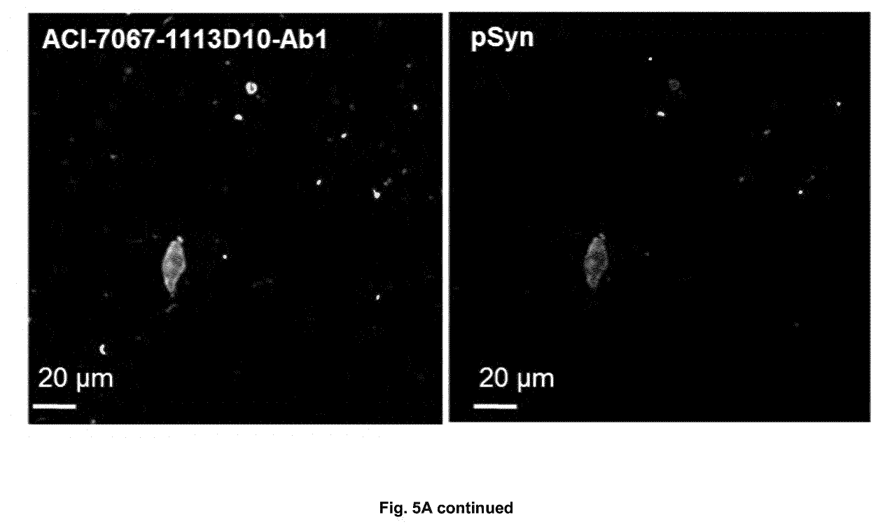

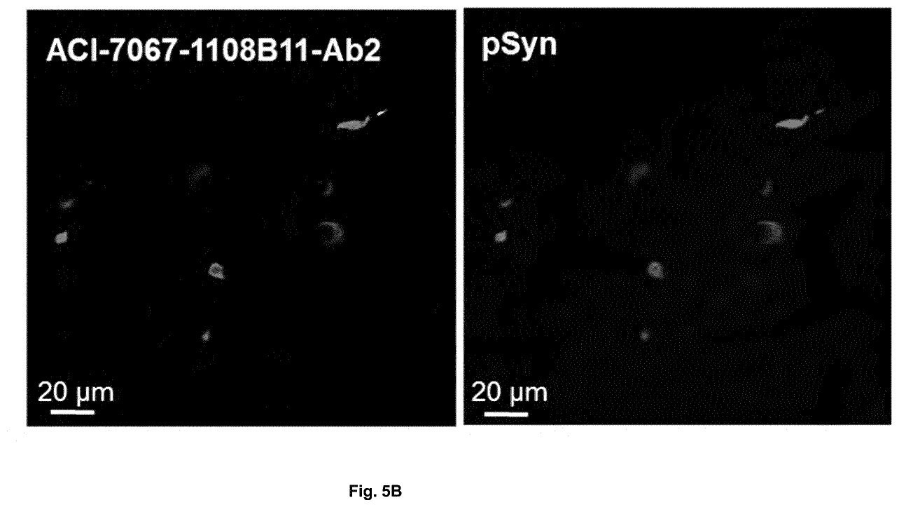

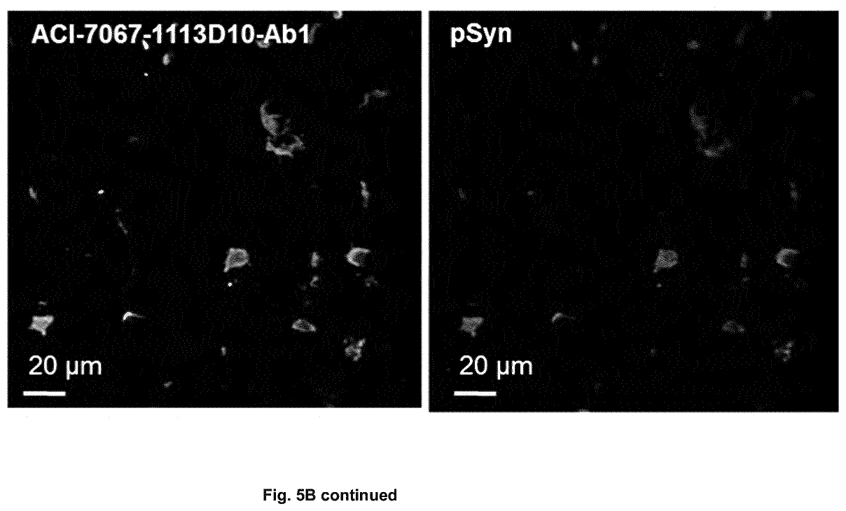

Target engagement was evaluated in immunohistochemistry experiments on tissues from PD and Multiple System Atrophy (MSA) donor brains. Human brain tissues were obtained from the Netherlands Brain Bank. All tissues have been collected from donors for or from whom a written informed consent for a brain autopsy and the use of the material and clinical information for research purposes had been obtained by the Netherlands Brain Bank. Immunohistochemistry was performed on 10 μm thick frozen sections using fluorescent secondary antibody detection. An antibody recognizing alpha-synuclein phosphorylated at Ser129, [EP1536Y] (pSyn) (Abcam ab51253) was used as control for detecting pathological aggregated and phosphorylated alpha-synuclein. Antibodies ACI-7067-1101C8-Ab2, ACI-7067-1113D10-Ab1 and ACI-7067-1108B11-Ab2 bind to pathological alpha-synuclein aggregates in Lewy bodies and Lewy neurites in PD cases (FIG. 5A) and in glial cytoplasmic inclusions in MSA cases (FIG. 5B). Similar results were obtained with other antibodies listed in Table 5 (data not shown).

### Antibody Variable Region Gene Sequencing

Clonal hybridoma cell lysates were used for variable region gene sequencing. Mouse hybridomas were harvested and lysed using a lysis buffer containing guanidinium salts that deactivates RNases. Genomic DNA was then eliminated by RNase-free DNase, and RNA was purified with a silica-based affinity column using multiple washes and eluted from the column using RNase-free water. Once the RNA was extracted, its purity and concentration was measured spectrophotometrically. The integrity of the RNA was assessed on a denaturing agarose gel and RNA was reverse transcribed into cDNA using reverse transcriptase (RT). Before adding the reaction mixture, the RNA was heated to 70° C. for 10 min in order to disrupt RNA secondary structures. The RT products were directly used for PCR amplification. For high-fidelity PCR amplification of the cDNA, each of the variable region primers corresponding to the different gene families encoding for antibodies were individually mixed with the constant primer, for variable heavy chain domain (VH) and variable light chain domain (VL) separately. In first intention, a degenerate primer pool was used (12 for VH and 12 for VL) and, depending on the results, a second pool was used to obtain PCR products. After the PCR reaction, the products were analyzed by gel electrophoresis on 2% agarose gels stained with ethidium bromide. The PCR products for VL and VH were individually purified on an agarose gel using tris-acetate-EDTA (TAE). The purified fragments excised from the gel were then sequenced using the dye-terminator sequencing method. The same primers as those used for PCR were used for the sequencing reaction. Sequencing was carried out in both directions to provide overlap at both ends. Sequencing data were analyzed on the Ig Blast/Kabat database. Nucleotide sequences for VH and VL are shown in Table 6. Protein sequences for VH and VL, and their complementarity-determining regions (CDRs) are shown in Table 7.

TABLE 6
Nucleotide sequence of the heavy chain and light chain variable domains (VH and VL)

| Antibody | Hybridoma | | |
|---|---|---|---|
| Code | Code | VH | VL |
| ACI-7067- | 1101C8F7 | GAGGTGCAGCTTGTTGAGTCTGGTGGAGGA | GATGTTTTGATGACCCAAACTCCACTCTCC |
| 1101C8- | | TTGGTGCAGCCTAAAGGGTCATTGAAACTC | CTGCCTGTCAGTCTTGGAGATCAAGCCTCC |
| Ab2 | | TCATGTGCAGCCTCTGGATTCAGCTTCAAT | ATCTCTTGCAGATCTAGTCAGAGCATTGTA |
| | | ATCTACGCCATGAACTGGGTCCGCCAGGCT | CATAGTAATGGAAACACCTATTTAGAATGG |
| | | CCAGGAAAGGGTTTGGAATGGGTTGCTCGC | TACTTGCAGAAACCAGGCCAGTCTCCAAAG |
| | | ATAAGAAGTAAAAGTAATAATTATGCAACA | CTCCTGATCTACAAAGTTTCCAACCGATTT |
| | | TATTATGCCGATTCAGTGAAAGACAGATTC | TCTGGGGTCCCAGACAGGTTCAGTGGCAGT |
| | | ACCATCTCCAGAGCTGATTCAGAAAGCATG | GGATCAGGGACAGATTTCACACTCAAGATC |
| | | CTCTATCTGCAAATGAACAACTTGAAAACT | AGCAGAGTGGAGGCTGAGGATCTGGGAGTT |
| | | GAGGACACAGCCATGTATTACTGTGTAAGG | TATTACTGCTTTCAAGGTTCACAAGGTCCG |
| | | GTGGGCCTACGGTTCTATGCTATGGACTAC | CTCACGTTCGGTGCTGGGACCAAGCTGGAG |
| | | TGGGGTCAAGGCACCTCAGTCACCGTCTCC | CTGAAA |
| | | TCA | (SEQ ID NO: 19) |
| | | (SEQ ID NO: 18) | |
| ACI-7067- | 1102G3F2 | GAAGTGAAGCTTGAGGAGTCTGGAGGAGGC | AGTATTGTGATGACCCAGACTCCCAAATTC |
| 1102G3- | | TTGGTGCAACCTGGAGGATCCATGAAACTC | CTGCTTGTATCAGCAGGAGACAGGGTTACC |
| Ab1 | | TCTTGTGCTGCCTCTGGATTCACTTTTAGT | ATAACCTGCAAGGCCAGTCAGAGTGTGACT |
| | | GACGCCTGGATGAACTGGGTCCGCCAGTCT | AAAGATGTAGCTTGGTACCAACAGAAGCCA |
| | | CCAGAGAAGGGGCTTGAGTGGGTTGCTGAA | GGGCAGTCTCCTAAACTGCTGATATACTCT |
| | | ATTAGAAACAAAGCTCATAATCATGCAACA | ACATCCAATCGCTACAGTGGAGTCCCTGAT |
| | | TACTATGCTGAGTCTGTGAAAGGGAGGTTC | CGCTTCACTGGCAGTGGATATGGGACGGAT |
| | | ACCATCTCAGGAGATGATTCCAAAAGTAGT | TTCACTTTCACCATCAATACTGTGCAGACT |
| | | GTCTACCTGCAAATGAACAACTTAAGAGCT | GAAGACCTGGCAGTTTATTTCTGTCAGCAG |
| | | GAAGACACTGGCATTTATTACTGTACCATT | GATTACAGGATTCCGTACACGTTCGGAGGG |
| | | TACTCTTATTGGGGCCAAGGGACTCTGGTC | GGGACCAAGCTGGAAATAAAA |
| | | ACTGTCTCTGCA | (SEQ ID NO: 29) |
| | | (SEQ ID NO: 28) | |
| ACI-7067- | 1106A8H3 | GAGGTGCAGCTTGTTGAGTCTGGTGGAGGA | CAAATTGTTCTCACCCAGTCTCCAGCAATC |
| 1106A8- | | TTGGTGCAGCCTAAAGGATCATTGAAACTC | ATGTCTGCATCTCCAGGGGAGAAGGTCACC |
| Ab2 | | TCATGTGCCGCCTCTGGTTTCACCTTCAAT | ATGACCTGCAGTGCCAGCTCAAGTGTAAGT |
| | | ACCTATGCCATGCACTGGGTCCGCCAGGCT | TACATGCACTGGTACCAGCAGAAGTCAGGC |
| | | CCAGGAAAGGGTTTGGAATGGGTTGCTCGC | ACCTCCCCCAAAAGATGGATTTATGACACA |
| | | ATAAGAAGTAAAGGTAGTAATTATGCAACA | TCCAATCTGGCTTCTGGAGTCCCTGCTCGC |
| | | AATTATGCCGATTCAGTGAAAGACAGATTC | TTCAGTGGCAGTGGGTCTGGGACCTCTTAC |
| | | ACCATCTCCAGAGATGATTCGCAAAGCATG | TCTCTCACAATCAGCAGCATGGAGGCTGAA |
| | | CTCTATCTGCAAATGAACAACCTGAAAACT | GATGCTGCCACTTATTACTGCCAGCAGTGG |
| | | GAGGACACAGCCATGTATTACTGTGTGAGA | AATAGTCACCCACCCACGTTCGGTGCTGGG |
| | | GGACACGGTAGTAGCTACTTTTCTTACTGG | ACCAAGCTGGAACTGAAA |
| | | GGCCAAGGGACTCTGGTCACTGTCTCTGCA | (SEQ ID NO: 39) |
| | | (SEQ ID NO: 38) | |
| ACI-7067- | 1107G5B6 | CAGGTCCAACTGCAGCAGCCTGGGACTGAA | GACATCCAGATGACCCAGTCTCCATCCTCC |
| 1107G5- | | CTGGTGAAGCCTGGGGCTTCAGTGAAGCTG | TTATCTGCCTTTCTGGGAGAAAGAGTCAGT |
| Ab2 | | TCCTGCAAGGCTTCTGGCTACACCTTCACC | CTCACTTGTCGGGCAAGTCAGGACATTGGT |
| | | AAATACTGGATGCACTGGGTGAAGCAGAGG | AATAACTTAAACTGGTTTCAGCAGGAACCA |
| | | CCTGGACAAGGCCTTGAGTGGATTGGAAAT | GATGGAACTATTAAACGTCTGATCTACGCC |
| | | ATTAATCCTAACAATGGTGATACTAACTAC | ACATCCAGTTTAGATTCTGGTGTCCCCAAA |
| | | AATGAGAAGTTCAAGAGCAAGGCCACACTG | AGGTTCAGTGGCAGTAGGTCTGGGTCAGAA |
| | | ACTGTAGACAAATCCTCCAGCACAGCCTAC | TATTCTCTCACCATCAGCAGCCTTGAGTCT |
| | | ATGCAGCTCAGCAGTCTGACATCTGAGGAC | GAAGATTTTGTAGACTATTACTGTCTACAA |
| | | TCTGCGGTCTATTATTGTGCAATTGCTATG | TTTGGTAGTTCTCCGCTCACGTTCGGTGCT |
| | | GACTACTGGGGTCAAGGAACCTCAGTCACC | GGGACCAAGCTGGAGCTGAAA |
| | | GTCTCCTCA | (SEQ ID NO: 49) |
| | | (SEQ ID NO: 48) | |
| ACI-7067- | 1108H1E1 | GAGGTGAAGCTGGTGGAGTCTGGAGGAGGC | AGTATTGTGATGACCCAGACTCCCAAATTC |
| 1108H1- | | TTGGTGCAACCTGGAGGATCCATGAAACTC | CTGCTTGTATCAGCAGGAGACAGGGTTACC |
| Ab1 | | TCTTGTACTGCCTCTGGATTCACTTTTAGT | ATAACCTGCAAGGCCAGTCAGAGTGTGACT |
| | | GACGCCTGGATGAACTGGGTCCGCCAGTCT | AATTATGTAGCTTGGTACCATCAGAAGCCA |
| | | CCAGAGAAGGGGCTTGAGTGGGTTGCTGAA | GGGCAGTCTCCTAAACTGCTGATATACTCT |
| | | ATTAGAAACAAAGCTCATAATCATGCAACA | GCATCCAATCGCTACAGTGGAGTCCCTGAT |
| | | AACTATGCTGAGTCTGTGAAGGGGAGGTTC | CGCTTCACTGGCAGTGGATATGGGACGGAT |
| | | ACCATCTCAGGAGATGATTCCAAAAGTAGT | TTCACTTTCACCATCAATACTGTGCAGACT |
| | | GTCTACCTGCAAATGAACAACTTAAGAGCT | GAAGACCTGGCAGTTTATTTCTGTCAGCAG |
| | | GAAGACACTGGCATTTATTACTGTACCATT | GATTACAGGATTCCGTACACGTTCGGAGGG |
| | | TACTCTTTTTGGGGCCAAGGGACTCTGGTC | GGGACTAAGCTGGAAATAAAA |
| | | ACTGTCTCTGCA | (SEQ ID NO: 59) |
| | | (SEQ ID NO: 58) | |
| ACI-7067- | 1111B12H1 | CAGGTCCAACTGCTGCAGCCTGGGACTGCA | GACATCCAGATGACCCAGTCTCCATCCTCC |
| 1111B12- | 0 | CTGGTGATGCCTGGGGCTTCAGTGAAGCTG | TTATCTGCCTCTCTGGGAGAAAGAGTCAGT |
| Ab2 | | TCCTGCAAGGCTTCTGGCTACACCTTCACC | CTCACATGTCGGGCAAGTCAGGACATTGGT |
| | | ACCTACTGGATGCACTGGGTGAAGCAGAGG | ATTAGCTTAAACTGGTTTCAGCAGGAACCA |
| | | CCTGGACAAGGCCTTGAGTGGATTGGAAAT | GATGGAACTATTAAACGCCTGATCTACGCC |
| | | ATTAATCCTATCAATGGTGGTAGTAACTAC | ACATCCAGTTTAGATTCTGGTGTCCCCAAA |
| | | AATGAGAAGTTCAAGAGCAAGGCCTCACTG | AGGTTCAGTGGCAATAGGTCTGGGTCAGAT |
| | | ACTGTAGACAAGTCCTCCAGCACAGCCTAC | TATTCTCTCACCATCAGTAGCCTTGAGTCT |
| | | ATGCAGCTCAGCAGCCTGACATCTGAGGAC | GAAGATTTTGCAGACTATTACTGTCTACAA |
| | | TCTGCGGTCTATTATTGTGTCATTGCTATG | TTTGCTAGTTCTCCGCTCACGTTCGGTGCT |
| | | GACTACTGGGGTCAAGGAACCTCAGTCACC | GGGACCAAGCTGGAGCTGAAA |
| | | GTCTCCTCA | (SEQ ID NO: 69) |
| | | (SEQ ID NO: 68) | |
| ACI-7067- | 1112H8C1 | GAAGTGAAGCTTGAGGAGTCTGGAGGAGGC | AGTATTGTGATGACCCAGACTCCCAAATTC |
| 1112H8- | 2 | TTGGTGCAACCTGGAGGATCCATGAAACTC | CTGCTTATGTCACCAGGAGACAGGGTTACC |
| Ab2 | | TCTTGTGCTGCCTCTGGATTCACTTTTACT | ATGACCTGCACGGCCAGTCAGAGTGTGAGT |
| | | GACGCCTGGATGAACTGGGTCCGCCAGTCT | AATTATGTGGCTTGGTACCAACAGAAGCCA |
| | | CCAGAAAAGGGGCTTGAGTGGATTGCTGAA | GGGCAGTCTCCTAAACTGCTGATATACTCT |
| | | ATTAGAAACAAAGCTCATAATTATGCAACA | GCATCCAATCGCTTCACTGGAGTCCCTGAT |
| | | TACTATGCTGAGTCTGTGAAAGGGAGGTTC | CGCTTCACTGGCAGTGGATATGGGACGGAT |
| | | GACATCTCAGGAGATGATTCCAAAAGTAGT | TTCACTTTCACCATCAACACTGTGCAGACT |
| | | GTCTACCTGCAAATGAACAACTTGAGAGTT | GAAGACATGGCAGTTTATTTCTGTCAGCAG |
| | | GAAGACACTGGCATTTATTACTGTACCATT | GATTACACCTCTCCGTACACGTTCGGGGGG |
| | | TACTCTTACTGGGGCCCAGGGACTCTGGTC | GGGACCAAGCTGGAAATAAAA |
| | | ACTGTCTCTGCA | (SEQ ID NO: 79) |
| | | (SEQ ID NO: 78) | |
| ACI-7067- | 1108B11D3 | GAGGTGCAGCTTGTTGAGTCTGGTGGAGGA | CAAATTGTTCTCACCCAGTCTCCAGCAATC |
| 1108B11- | | TTGGTGCAGCCTAAAGGATCATTGAAACTC | ATGTCTGCATCTCCAGGGGAGAGGATCACC |
| Ab2 | | TCATGTGCCGCCTCTGGTTTCACCTTCAAT | ATGACCTGCAGTGCCAACTCAAGTGTTACT |
| | | ACCTATGCCATGCACTGGGTCCGCCAGGCT | TACATGCACTGGTACCAGCAGAAGTCAGGC |
| | | CCAGGAAAGGGTTTGGAATGGGTTGCTCGC | ACCTCCCCCAAAAGATGGATTTATGACACA |
| | | ATAAGAAGTAAAGGTAGTAATTATGCAACA | TCCAATCTGGCTTCTGGAGTCCCTGCTCGC |
| | | AATTATGCCGATTCAGTGAAAGACAGATTC | TTCAGTGGCAGTGGGTCTGGGACCTCTTAC |
| | | ACCATCTCCAGAGATGATTCGCAAAGCATG | TCTCTCACAATCAGCAGCATGGAGGCTGAA |
| | | CTCTATCTGCAAATGAACAACCTGAAAACT | GATGCTGCCACTTATTACTGCCAGCAGTGG |
| | | GAGGACACAGCCATGTATTACTGTGTGAGA | AAAAGTCACCCACCCACGTTCGGTGCTGGG |
| | | GGACACGGTAGTAGCTACTTTTCTTACTGG | ACCAAGCTGGAACTGAAA |
| | | GGCCAAGGGACTCTGGTCACTGTCTCTGCA | (SEQ ID NO: 89) |
| | | (SEQ ID NO: 38) | |
| ACI-7067- | 1113D10E3 | GAGGTGCAGCTTGTTGAGTCTGGTGGAGGA | CAAATTGTTCTCACCCAGTCTCCAGCAATC |
| 1113D10- | D5 | TTGGTGCAGCCTAAAGGATCATTGAAACTC | ATGTCTGCATCTCCAGGGGAGAAGGTCACC |
| Ab1 | | TCATGTGCCGCCTCTGGTTTCACCTTCAAT | ATGACCTGCAGTGCCAGCTCAAGTGTAAGT |
| | | ACCTATGCCCTGCACTGGGTCCGCCAGGCT | TACATGCACTGGTACCAGCAGAAGTCAGGC |
| | | CCAGGAAAGGGTTTGGAATGGGTTGCTCGC | ACCTCCCCCAAAAGATGGATTTATGACACA |
| | | ATAAGAAGTAAAAGTAGTAATTATGCAACA | TCCAAACTGGCTTCTGGAGTCCCTGCTCGC |
| | | TATTATGCCGATTCAGTGAAAGACAGATTC | TTCAGTGGCAGTGGGTCTGGGACCTCTTAC |
| | | ACCATCTCCAGAGATGATTCACAAAGCATG | TCTCTCACAATCAGCAGCATGGAGGCTGAA |
| | | CTCTATCTGCAAATGAACAACCTGAAAACT | GATTCTGCCACTTATTACTGCCAGCAGTGG |
| | | GAGGACACAGGCATGTATTACTGTGTAAGA | AGTAATAACCCACCGACGTTCGGTGGAGGC |
| | | GGGGGTGTTTCTCCCTTTGACTACTGGGGC | ACCAAGCTGGAAATCAAA |
| | | CAAGGCACCACTCTCACAGTCTCCTCA | (SEQ ID NO: 99) |
| | | (SEQ ID NO: 98) | |
| ACI-7067- | 1116F2A2 | GATGTACAACTTCAGGAGTCAGGACCTGGC | GATGTTGTGATGACCCAGACTGCACTCACT |
| 1116F2- | | TTCGTGAAACCTTCTCAGTCTCTGTCTCTC | TTGTCGGTTACCATTGGACAACCAGCCTCC |
| Ab1 | | ACCTGCTCTGTCACTGGCTACTCAATAACC | ATCTCTTGCAAGTCAAGTCAAAGCCTCTTA |
| | | AGAGGTTTTTACTGGAACTGGATCCGACAG | GATAGTGATGGAGAGACATATTTGAATTGG |
| | | TTTCCAGGAAACAAACTGGAATGGATGGGC | TTGTTACAGAGGCCAGGCCAGTCTCCAAAG |
| | | TACATAAGTGACGATGGTAATAGTAACTAC | CGCCTAATCTATCTGGTGTCTAAACTGGAC |
| | | AATCCCTCTCTCAAAAATCGAATCTCCATC | TCTGGAGTCCCTGACAGGTTCACTGGTAGT |
| | | ACTCGTGACACATTTAAGAATCAGGTTTTC | GGATCAGGGACAGATTTCGCACTGAAAATC |
| | | CTGAGGTTGAACTCTGTGACTACTGAGGAC | AGCAGAGTGGAGGCTGAGGACTTGGGAATT |
| | | ACTGCCACATACTATTGTACAAGAGGAGAT | TATTATTGCTGGCAAGGTACACATTTTCCT |
| | | CTACTTTGGGGCCAAGGCACCACTCTCACA | CAGACGTTCGGTGGAGGCACCAAGCTGGAA |
| | | GTCTCCTCA | ATCAAA |
| | | (SEQ ID NO: 108) | (SEQ ID NO: 109) |
| ACI-7067- | 1206E5D2 | CAGGTTCAGCTGCAGCAGTCTGGACCTGAG | GATGTTTTGATGACCCAAACTCCACTCACT |
| 1206E5- | | CTGGTGAAGCCTGGGGCTTCAGTGAAGATG | TTGTCGGTTACCATTGGACAACCAGCCTCT |
| Ab1 | | TCCTGCAAGGCTTCTGGATACACATTCACT | ATCTCTTGCAAGTCAAGTCAGAGCCTCTTA |
| | | GACTATGTTATAAGCTGGGTGAAGCAGGGA | TATAGTAATGGAAAAACCTATTTGAATTGG |
| | | ACTGGACAGGGCCTTGAGTGGATTGGAGAG | TTATTACAGAGGCCAGGCCAGTCTCCAAAG |
| | | ATTTATCCTGGAAATGATAGTACTTACTAC | CGCCTAATCTATCTGGTGTCTAAACTGGAC |
| | | AATGAGAAGTTCAAGGGCAAGGCCACACTG | TCTGGAGTCCCTGACAGGTTCACTGGCAGT |
| | | ACTGCAGACAAATCCTCCAACACAGCCTAC | GGATCAGGAACAGATTTTACACTGAAAATC |
| | | ATGCAGCTCAGCAGCCTGACATCTGAGGAC | AGCAGAGTGGAGGCTGAGGATTTGGGAGTT |
| | | TCTGCGGTCTATTTCTGTGCAAGAGAGGGG | TATTACTGCGTGCAAGGTACACATTTTCCG |
| | | GTCTCTAATGGTTACCTATATTTGTCTATG | TGGACGTTCGGTGGAGGCACCAAGCTGGAA |
| | | GACTACTGGGGTCAAGGAACCTCAGTCACC | ATCAAA |
| | | GTCTCCTCA | (SEQ ID NO: 119) |
| | | (SEQ ID NO: 118) | |
| ACI-7079- | 2501B11C7 | CAGGTTCAGCTGCAGCAGTCTGGACCTGAG | CAGGCTGTTGTGACTCAGGAATCTGCACTC |
| 2501B11- | | CTGGTGAAGCCTGGGGCCTCAGTGAAGATT | ACCACATCACCTGGTGAAACAGTCACACTC |
| Ab3 | | TCCTGCAAGGCTTCTGGCTACGCATTCAGT | ACTTGTCGCTCAAGTACTGGGGCTGTTACA |
| | | AGTTTCTGGATGAACTGGATGAAACAGAGG | ACTAGTAACTATGCCAACTGGGTCCAAGAA |
| | | CCTGGAAAGGGTCTTGAGTGGATTGGACGG | AAACCAGATCATTTATTCACTGGTCTAATA |
| | | ATTTATCCTGGAGATGGAGATGCTCACTAC | GGTGGTACCAACAACCGAGCTCCAGGTGTT |
| | | AATGGGGAGTTCAAGGGCAGGGCCACACTG | CCTGCCAGATTCTCAGGCTCCCTGATTGGA |
| | | ACTGCAGACAAATCCTCCAGCACAGCCTAC | GACAAGGCTGCCCTCACCATCACAGGGGCA |
| | | ATGCAACTCAGCAGCCTGACATCTGAGGAC | CAGACTGAGGATGAGGCAATATATTTCTGT |
| | | TCTGCGGTCTACTTCTGTGCAAGAAAGGGG | GCTCTATGGTACAGCAACCATTTGGTGTTC |
| | | GATTTCTACGGTAGTAACTACGACTATTGG | GGTGGAGGAACCAGACTGACTGTCCTA |
| | | GGCCAAGGCACCACTCTCACAGTCTCCTCA | (SEQ ID NO: 289) |
| | | (SEQ ID NO: 288) | |
| ACI-7079- | 2501D10C | GAGGTGCAGCTTGTTGAGTCTGGTGGAGGA | CAAATTGTTCTCACCCAGTCTCCAGCAATC |
| 2501D10- | 3 | TTGGTGCAGCCTAAAGGATCATTGAAAGTC | ATGTCTGCATTTCCAGGGGAGAGGGTCACC |
| Ab1 | | TCATGTGCCGCCTCTGGTTTCACCTTCAAG | ATGACCTGCAGTGCCAGCTCAAGTGTAAAT |
| | | ACCTATGCCATGCACTGGGTCCGCCAGGCT | TACATGCACTGGTACCAGCAGAAGTCCGGC |
| | | CCGGGAAAGGGTTTGGAATGGGTTGCTCGC | ACCTCCCCCAAAAGATGGATTTATGACACA |
| | | ATAAGAAGTGAAAACAGTAATTTTGCAAAA | TCCAAACTGGCTTCTGGAGTCCCTGCTCGC |
| | | TATTATGCCGATTCAGTGAAGGACAGATTC | TTCAGTGGCGGTGGGTCTGGGACCTCTTAC |
| | | ACCATCTCCAGAGATGATTCACAAAGTATG | TCTCTCACAATCAGCAACATGGAGGCTGAA |
| | | CTCTATCTGCAAATGAACAACCTGAAAACT | GATGCTGCCACTTATTACTGCCAGCAGTGG |
| | | GAGGACACAGCCATGTATTATTGTGTAAGG | AGAAGTAATCCACCCACTTTCGGAGGGGGG |
| | | GGATATAACGGCAGTAGCCTTGACTACTGG | ACCAAGCTGGAAATAAAA |
| | | GGCCAAGGCACCACTCTCACAGTCTCCTCA | (SEQ ID NO: 199) |
| | | (SEQ ID NO: 298) | |
| ACI-7079- | 2501G2E5 | GAGGTGCAGCTTGTTGAGTCTGGTGGAGGA | GATGTTTTGATGACCCAAACTCCACTCTCC |
| 2501G2- | | TTGGTGCAGCCTAAAGGGTCATTGAAACTC | CTGCCTGTCAGTCTTGGAGATCAAGTCTCC |
| Ab2 | | TCATGTGCAGCCTCTGGATTCAACTTCAAT | ATCTCTTGCAGATCTAGTCAAACCATTGTA |
| | | ACCTATGCCATGAACTGGGTCCGCCAGGCT | CATAGTAATGGAAACACCTATTTAGAATGG |
| | | CCAGGAAAGGGTTTGGAATGGGTTGCTCGC | TACCTGCAGAAACCAGGCCAGTCTCCAAAG |
| | | ATAAGAACTAAAAGTAATAATTTTGCAACA | CTCCTGATCTACAAAGTTTCCAACCGATTT |
| | | TATTATGCCCATTCAGTGAAAGACAGATTC | TCTGGGGTCCCAGACAGGTTCAGTGGCAGT |
| | | ACCATCTCCAGAGATGATTCAGAAAGCATG | GGATCAGGGACAGATTTCACACTCAAGATC |
| | | CTCTATCTGCAAATGAACAACTTGAAAACT | AGCAGAGTGGAGGCTGAGGATCTGGGAGTT |
| | | GAGGACACAGCCATGTATTACTGTGTGAGA | TATTACTGCTTTCAAGGTTCACAAGGTCCG |
| | | CAGGGACTAGCCTACTATGCTATGGACTAC | CTCACGTTCGGTGCTGGGACCAAACTGGAG |
| | | TGGGGTCAAGGAACCTCAGTCACCGTCTCC | CTGAAA |
| | | TCA | (SEQ ID NO: 149) |
| | | (SEQ ID NO: 148) | |
| ACI-7079- | 2503C6H9 | GATGTACAGCTTCAGGAGTCAGGACCTGGC | GATGTTGTGATGACCCAGACTCCACTCACT |
| 2503C6- | | CTCGTGAAACCTTCTCAGTCTCTGTCTCTC | TTGTCGGTTACCATTGGACAACCAGCCTCC |
| Ab1 | | ACCTGCTCTGTCACTGGCTACTCCATCACC | ATCTCTTGCAAGTCAAGTCAGAGCCTCTTA |
| | | AGTGGTTATTACTGGAACTGGATCCGACTA | GATAGTGATGGAGAGACATATTTGAATTGG |
| | | TTTCCAGGAAACAAACTGGAATGGCTGGGC | TTGTTACAGAGGCCAGGCCAGTCTCCAAAG |
| | | TACATAAACTACGATGGTAGCAATAACTTC | CGCCTAATCTGTCTGGTGTCTAAACTGGAC |
| | | AACCCATCTCTCAAAAATCGAATCTCCATC | TCTGGAGTCCCTGACAGGTTCACTGGCAGT |
| | | ACTCGTGACACATCTAAGAACCAGTTTTTC | GGATCAGGGACAGATTTCACACTGAAAATC |
| | | CTGAAATTGAATTCTGTGACTTCTGAGGAC | AGCAGAGTGGAGGCTGAGGATTTGGGAGTT |
| | | ACAGCCACATATTTCTGTTTAAGAGGGGAC | TATTATTGCTGGCAAGGTACACATTTTCCT |
| | | TGGGACTGGGGCCAAGGGACTCTGGTCACT | CAGACGTTCGGTGGAGGCACCAGGCTGGAA |
| | | GTCTCTGCA | ATCAAA |
| | | (SEQ ID NO: 158) | (SEQ ID NO: 159) |
| ACI-7079- | 2504A6C8 | CAGGTTCAGCTGCAGCAGTCTGGAGTTGAG | GATGTTGTGATGACCCAAACTCCACTCTCC |
| 2504A6- | | CTGGCGAGGCCTGGGGCTTCAGTGAAACTG | CTGCCTGTCAGTCTTGGAGATCAAGCCTCC |
| Ab1 | | TCCTGCAAGGCTTCTGGCTACACCTTCACA | ATCTCTTGCAGATCTAGTCAGAGCCTTGTA |
| | | AGCTATGGTATAAGCTGGGTGAAGCAGAGA | CACAGTAATGGAAACACCTATTTACATTGG |
| | | ACTGGACAGGGCCTTAAGTGGATTGGAGAG | TACCTGCAGAAGCCAGGCCAGTCTCCAAAG |
| | | ATTTATCCTGGAAGTGGTAATACTTACTAC | CTCCTGATCTACAAAGTTTCCAACCGATTT |
| | | AATGAGAAGTTCAAGGGCAAGGCCACACTG | TCTGGGGTCCCAGACAGGTTCAGTGGCAGT |
| | | ACTGCAGACAAATCCTCCAGCACAGCGTAC | GGATCAGGGACAGATTTCACACTCAAGATC |
| | | ATGGAGCTCCGCAGCCTGACGTCTGAGGAC | AGCAGAGTGGAGGCTGAGGATCTGGGAGTT |
| | | TCTGCGGTCTATTTCTGTGCAACCGATTAC | TATTTCTGCTCTCAAAGTACACATGTTCCG |
| | | GACGCCTACTGGGGCCAAGGCACCACTCTC | CTCACGTTCGGTGCTGGGACCAAGCTGGAG |
| | | ACAGTCTCCTCA | CTGAAA |
| | | (SEQ ID NO: 168) | (SEQ ID NO: 169) |
| ACI-7079- | 2506E2G4 | CAGGTTCAGTTGCAGCAGTCTGGACCTGAG | GACATTGTGCTGACACAGTCTCCTGCTTCC |
| 2506E2- | | CTGGTGAGGCCTGGGGCCTCAGTGAAGATT | TTAACTGTATCTCTGGGGCAGAGGGCCACC |
| Ab2 | | TCCTGCAAGGCTTCTGGCTACGCATTCAGT | ATCTCATGCAGGGCCAGCCAAAGTGTCAGT |
| | | AACTCCTGGATGAACTGGGTGAAGCAGAGG | ACATCTAGGAATAGTTATATGCACTGGTAC |
| | | CCTGGAAAGGGTCTTGAGTGGATTGGACGG | CAACAGAAACCAAGACAGCCACCCAAACTC |
| | | ATTTTTCCTGGAGATGGAGATACTTACTAC | CTCATCAAGTATGCATCCAACCTAGAATCT |
| | | GATGGGAAGTTCAAGGGCAAGGTCAAACTG | GGGGTCCCTGCCAGGTTCAGTGGCAGTGGG |
| | | ACAACAGACAAATTCTCCAACACAGCCTAC | TCTGGGGCAGACTTCACCCTCAACATCCAT |
| | | ATGCAACTCCGCAGCCTGACATCTGAGGAC | CCTGTGGAGGAGGAGGATACTGCAACATAT |
| | | TCTGCGGTCTACTTCTGTGCAAGATGGGGG | TACTGTCAGCACAGTTGGGATATTCCGCTC |
| | | GGTACTAACGATGAGTGGTTTGCTCACTGG | ACGTTCGGTACTGGGACCAAGCTGGAGCTG |
| | | GGCCAAGGGACTCTGGTCACTGTCTCTGTA | AGT |
| | | | (SEQ ID NO: 179) |
| | | (SEQ ID NO: 178) | |
| ACI-7079- | 2506F3E12 | CAGGTCCAACTGCAGCAGCCTGGGGCTGAG | GATGTTTTGATGACCCAAACTCCACTCTCC |
| 2506F3- | | CTTGTGAAGCCTGGGGCTTCAGTGAAGCTG | CTGCCTGTCAGTCTTGGAGATCAAGCCTCC |
| Ab1 | | TCCTGCAAGGCTTCTGGCTACACCTTCACC | ATCTCTTGCAGATCTAGTCAGAGCATTGTA |
| | | ACCTACTGGATGCAGTGGGTAAAACAGAGG | CATAGTAATGGAAACACCTATTTAGAATGG |
| | | CCTGGACAGGGCCTTGAGTGGATCGGAGAG | TACCTGCAGAAACCAGGCCAGTCTCCAAAG |
| | | ATTGATCCTTCTGATAGCTATATTAACTAC | CTCCTGATCTACAAAGTTTCCAACCGATTT |
| | | AATCAAAAGTTCAAGGGCAAGGCCACATTG | TCTGGGGTCCCAGACAGGTTCAGTGGCAGT |
| | | ACTGTAGACACATCCTCCAGCACAGCCTTC | GGATCAGGGACAGATTTCACACTCAAGATC |
| | | ATGCAGCTCAGCAGCCTGACATCTGAGGAC | AGCAGAGTGGAGGCTGAGGATCTGGGAGTT |
| | | TCTGCGGTCTATTACTGTGCAAGGGGGATG | TATTACTGCTTTAAAGGTTCACATGTTCCG |
| | | ATGGACTACTGGGGTCAAGGAACCTCAGTC | TACACGTTCGGAGGGGGGACCAAGCTGGAA |
| | | ACCGTCTCCTCA | ATAAAA |
| | | (SEQ ID NO: 188) | (SEQ ID NO: 189) |
| ACI-7079- | 2507B3G8 | GAGGTGCAGCTTGTTGAGTCTGGTGGAGGA | CAAATTGTTCTCACCCAGTCTCCAGCAATC |
| 2507B3- | | TTGGTGCAGCCTAAAGGATCATTGAAAGTC | ATGTCTGCATTTCCAGGGGAGAGGGTCACC |
| Ab1 | | TCATGTGCCGCCTCTGGTTTCACCTTCAAG | ATGACCTGCAGTGCCAGCTCAAGTGTAAAT |
| | | ACCTATGCCATGCACTGGGTCCGCCAGGCT | TACATGCACTGGTACCAGCAGAAGTCCGGC |
| | | CCGGGAAAGGGTTTGGAATGGGTTGCTCGC | ACCTCCCCCAAAAGATGGATTTATGACACA |
| | | ATAAGAAGTGAAAACAGTAATTTTGCAAAA | TCCAAACTGGCTTCTGGAGTCCCTGCTCGC |
| | | TATTATGCCGATTCAGTGAAAGACAGATTC | TTCAGTGGCGGTGGGTCTGGGACCTCTTAC |
| | | ACCATCTCCAGAGATGATTCACAAAGTATG | TCTCTCACAATCAGCAACATGGAGGCTGAA |
| | | CTCTATCTGCAAATGCACACCCTGAAAACT | GATGCTGCCACTTATTACTGCCAGCAGTGG |
| | | GAGGACACAGCCATCTATTATTGTGTAAGG | AGAAGTAATCCACCCACTTTCGGAGGGGGG |
| | | GGATATAACGGCAGTAGCCTTGACTACTGG | ACCAAGCTGGAAATAAAA |
| | | GGCCAAGGCACCACTCTCACAGTCTCCTCA | (SEQ ID NO: 199) |
| | | (SEQ ID NO: 198) | |
| ACI-7079- | 2511B3B12 | GATGTACAGCTTCAGGAATCAGGACCTGGC | GATGTTGTGATGACCCAGACTCCACTCACT |
| 2511B3- | | CTCGTGAAACCTTCTCAGTCTCTGTCTCTC | TTGTCGCTTACCATTGGACAACCAGCCTCC |
| Ab3 | | ACCTGCTCTGTCACTGGCTTCTCCATCACC | ATCTCTTGCAAGTCAAGTCAGAGCCTCTTA |
| | | AGTTATTATTACTGGAACTGGATCCGGCAG | GATAGTGATGGAGAGACATATTTGAATTGG |
| | | TTTCCAGGAAACAAACTGGAATGGATGGCC | TTGTTACAGAGGCCAGGTCAGTCTCCAAAG |
| | | TACATAAGCTACGATGGTAGCAATAACTAC | CGCCTAATCTATCTGGTGTCTAAACTGGAA |
| | | AACCCATCTCTCAAAAATCGAATCTCCATC | TCTGGAGTCCCTGACAGGTTCACTGGCAGT |
| | | ACTCGTGACACATCTAAGAACCAGTTTTTC | GGATCAGGGACAGTTTTCACACTGAAAATC |
| | | CTGAAGTTGAATTCTGTGACTACTGAGGAC | AGCAGAGTGGAGGCTGAGGATTTGGGAGTT |
| | | ACAGCCACATATTACTGTACAAGAGGGGAC | TATTATTGCTGGCAAGGGACACATTTTCCT |
| | | TGGGACTGGGGCCAAGGGACTCTGGTCACT | CAGACGTTCGGTGGAGGCACCAAGCTGGAA |
| | | GTCTCTGCA | ATCAAA |
| | | (SEQ ID NO: 208) | (SEQ ID NO: 209) |
| ACI-7079- | 2601B6D2 | GAGATTCAACTGCAGCAGTCTGGGGCTGAG | GACATTGTGATGACCCAGTCTCACAAATTC |
| 2601B6- | | CTTGTGAGGCCAGGGGCCTCAGTCAAGTTG | ATGTCCACATCAGTAGGAGACAGGGTCAGC |
| Ab1 | | TCCTGCACAACTTCCGGCTTTAACATTAAA | ATCACCTGCAAGGCCAGTCAGGATGTGGGT |
| | | GACGACTATATTCACTGGGTGAAGCAGAGG | AATGTTGTTGCCTGGTATCAACAGAAACCA |
| | | CCTGAACAGGGCCTGGAGTGGATTGGATGG | GGACAATCTCCTAAACTACTGATTTACTGG |
| | | ATTGATCCTGAGAATGGTGATACTGATTAT | GCATCCTCCCGGCACACTGGAGTCCCTGAT |
| | | GCCTCGAAGTTCCAGGGCAAGGCCACTATA | CGCTTCACAGGCAGTGGATCTGGGACAGAA |
| | | ACAGCAGACACATCCTCCAACACAGCCTAC | TTCACTCTCACCATTAGCAATGTGCAGTCT |
| | | CTGCACCTCAGCAGCCTGACATCAGAGGAC | GAAGACTTGGCAGATTATTTCTGTCAGCAA |
| | | GCTGCCGTCTATTTCTGTACTACAAGAGGA | TATAGCAGCTATCCGCTCACGTTCGGTGCT |
| | | TTTGGTTACTGGGGCCAAGGGACTCTGGTC | GGGACCAAGCTGGAGCTGAAG |
| | | ACTGTCTCT | (SEQ ID NO: 219) |
| | | (SEQ ID NO: 218) | |
| ACI-7079- | 2602G4H1 | GAGGTGCAGCTTGTTGAGTCTGGTGGAGGA | CAAATTGTTCTCACCCAGTCTCCAGCAATC |
| 2602G4- | | TTGGTGCAGCCTAAAGGATCATTGAAACTC | ATGTCTGCATCTCCAGGGGAGAGGATCACC |
| Ab4 | | TCATGTGCCGCCTCTGGTTTCACCTTCAAG | ATGACCTGCACTGCCAGCTCAAGTGTAAGT |
| | | ACCTATGCCATGCACTGGGTCCGCCAGGCT | TACATGCACTGGTACCAGCAGAAGTCAGGC |
| | | CCAGGAAAGGGTTTGGAATGGGTTGCTCGC | ACCTCCCCCAAAAGATGGATTTATGACACA |
| | | ATAAGAAGTAAAGGTAGTGATTATGCAACA | TCCAAACTGGCTTCTGGAGTCCCTGCTCGC |
| | | TATTATGCCGATTCAGTGAAGGACAGATTC | TTCAGTGGCAGTGGGTCTGGGGCCTCTTAT |
| | | ACCATCTCCAGAGATGATTCACAAAGCATG | ACTCTCACAATCAGCAGCATGGAGGCTGAA |
| | | CTCTATCTGCAAATGAACAACCTGAAAACT | GATGCTGCCACTTATTACTGCCAGCAGTGG |
| | | GAGGATACAGCCATGTATTTCTGTGTGAGA | AATCGTAACCCACCGACGTTCGGTGGAGGC |
| | | GGGGGTGCTGACTCCTGGTTTGCTTACTGG | ACCCAGCTGGCAATCAAA |
| | | GGCCAAGGGACTCTGGTCACTGTCTCTACA | (SEQ ID NO: 229) |
| | | (SEQ ID NO: 228) | |
| ACI-7079- | 2603C1H6 | CAGGTCCAACTGCAGCAACCTGGGGCTGAC | GAAAATGTTCTCACCCAGTCTCCAGCAATC |
| 2603C1- | | CTTGTGAAGCCTGGGGCTTCAGTGAAGCTG | ATGTCTGCATCTCCAGGGGAAAAGGTCACC |
| Ab3 | | TCCTGTAAGGCTTCTGGCTACACCTTCACC | ATGACCTGCAGTGCCGGCTCAAGTGTAAGT |
| | | AGTTACTGGATGCAGTGGACAAAACAGAGG | TACATGCACTGGTTCCAACAGAAGTCAAGC |
| | | CCTGGACAGGGCCTTGAGTGGATCGGAGAG | ACCTCCCCCAAACTCTGGATTTATGACACA |
| | | ATTGATCCTTCTGATAGCTATGCTAACTAC | TCCAAACTGCCTTCTGGAGTCCCAGGTCGC |
| | | AATCAAAAGTTCAAGGGCAAGGCCACATTG | TTCAGTGGCAGTGGGTCTGGAAACTCTTAC |
| | | ACTGTTGACAAATATTCCAGCACAGCCTAC | TCTCTCACGATCAGCAGCATGGAGGCTGAA |
| | | ATGCAGCTCAACAGCCTGACATCTGAGGAC | GATGTTGCCACTTATTACTGTTTTCAGGGG |
| | | TCTGCGGTCTATTACTGTGCCCTCTATGAT | AGTGGGTACCCGTACACGTTCGGAGGGGGG |
| | | GGTCCCTCTTACTGGGGCCAAGGGACTCTG | ACCAAGCTGGAAATAAAA |
| | | GTCACTGTCTCT | |
| | | (SEQ ID NO: 238) | (SEQ ID NO: 239) |
| ACI-7079- | 2603F3H3 | GAGGTGCAGCTTGTTGAGTCTGGTGGAGGA | CAAATTGTTCTCACCCAGTCTCCAGCAATC |
| 2603F3- | | TTGGTGCAGCCTAAAGGATCATTGAAACTC | ATGTCTGCATCTCCAGGGGAGAGGGTCACC |
| Ab1 | | TCATGTGCCGCCTCTGGTTTCACCTTCAAT | ATGACCTGCACTGCCAGCTCAAGTGTAAGT |
| | | ACCTATGCCATGCACTGGGTCCGCCAGGCT | TACATGCACTGGTACCAGCAGAAGTCAGGC |
| | | CCAGGAAAGGGTTTGGAATGGGTTGCTCGC | ACCTCCCCCAAAAGATGGATTTATGACACA |
| | | ATAAGAAGTAAAGGTAGTAATTATGCAACA | TCCAAACTGGCTTCTGGAGTCCCTGCTCGC |
| | | TATTATGCCGATTCAGTGAAAGACAGATTC | TTCAGTGGCAGTGGGTCTGGGGCCTCTTAT |
| | | ACCATCTCCAGAGATGATTCACAAAGCATG | ACTCTCACAATCAGCAGCATGGAGGCTGAA |
| | | CTCTATCTGCAAATGAACAACCTGAAAACT | GATGCTGCCACTTATTACTGCCAGCAGTGG |
| | | GAGGACACAGCCATGTATTACTGTGTGAGA | AATAGTAACCCACCGACGTTCGGTGGAGGC |
| | | GGGGGTGGTGACTCCTGGTTTGCTTACTGG | ACCCAGCTGGCAATCAAA |
| | | GGCCAAGGGACTCTGGTCACTGTCTCTGCA | (SEQ ID NO: 249) |
| | | (SEQ ID NO: 248) | |
| ACI-7079- | 2605B3D1 | GAGGTGCAGCTTGTTGAGTCTGGTGGAGGA | CAAATTGTTCTCACCCAGTCTCCAGCAATC |
| 2605B3- | | TTGGTGCAGCCTAAAGGATCATTGAAACTC | ATGTCTGCTTCTCCAGGGGAGAAGGTCACC |
| Ab2 | | TCATGTGCCGCCTCTGGTTTCATCTTTAAA | ATGACCTGCAGTGCCAGCTCAAGTGTAACT |
| | | ACCTATGCCATGCATTGGGTCCGCCAGGCT | TACATGCATTGGTACCAGCAGAAGTCAGGC |
| | | CCAGGAAAGGGTTTGGAATGGGTTGCTCGA | ACCTCCCCCAAAAGATGGATTTATGACACA |
| | | ATAAGAAGTAAAGGTGGTAATTATGCAACA | TCCCAACTGGCTTCTGGAGTCCCTGCTCGC |
| | | TATTTTGCCGATTCAGTGAAAGACAGATTC | TTCAGTGGCAGTGGGTCTGGGACCTCTCAC |
| | | ACCATCTCCAGAGATGATTCACAAAATATG | TCTCTCACAATCAGCAGCATGGAGACTGAA |
| | | CTCTATCTGCAAGTGAACAACCTGAAAATT | GATGCTGCCACTTATTACTGCCAACAATGG |
| | | GAGGACACAGCCATGTATTTCTGTGTGAGA | ACTAGAAACCCACCGACGTTCGGTGGAGGC |
| | | GGGGGTAATTACTCCTGGTTTGCTTACTGG | ACCAAGCTGGCAATCAAA |
| | | GGCCAAGGGACTCTGGTCACTGTCTCTGCA | (SEQ ID NO: 259) |
| | | (SEQ ID NO: 258) | |
| ACI-7079- | 2606A6D5 | CAGGTTCAGCTGCAACAGTCTGGACCTGAG | GACATTGTGCTGACACAGTCTCCTGCTTCC |
| 2606A6- | | CTGGTGAAGCCTGGGGCCTCAGTGAAGATT | TTAGCTGTATCTCTGGGGCAGAGGGCCACC |
| Ab2 | | TCCTGCAAGGCTTCTGGCTTCGCATTCAGT | ATCTCATGCAGGGCCAGCCAAAGTGTCAGT |
| | | AGCTCCTGGATGAACTGGGTGAAGCAGAGG | ACATCTAACTATAATTATCTTCACTGGTAC |
| | | CCTGGAAAGGGTCTTGAGTGGGTTGGACGG | CAACAGAAACCAGGACAGCCACCCAAACTC |
| | | ATTTTTCCTGGAGATGGAGATACTAACTAC | CTCATCACGTATGCATCCAACCTAGAATCT |
| | | GATAGGAAGTTCAAGGACAAGGCCACACTG | GGGGTCCCTGCCAGGTTCAGTGGCAGTGGG |
| | | ACTGCAGACAAATCCTCCAGCACAGCCTAC | TCTGGGACAGACTTCACCCTCAACATCCAT |
| | | ATGCAACTCAGCAGCCTGACATCTGAGGAC | CCTGTGGAGGAGGGAGATACTGCAACATAT |
| | | TCTGCGGTCTACTTCTGTGCAAGATGGACG | TACTGTCAACACAGTTGGGAGATTCCGCTC |
| | | GGGGGTTACGACTGGTTTGCTTACTGGGGC | ACGTTCGGTGCTGGGACCAAGCTGGAGCTG |
| | | CAAGGGACTCTGGTCACTGTCTCTGCA | AAA |
| | | (SEQ ID NO: 268) | (SEQ ID NO: 269) |
| ACI-7079- | 2509E5E5 | GAGGTCCAGCTGCAACAGTCTGGACCTGAA | GACATTGTGCTGACCCAATCTCCAGCTTCT |
| 2509E5- | | CTGGTGAAGCCTGGGGCTTCGCTGAAGATG | TTGGCTGTGTCTCTAGGGCAGAGGGCCACC |
| Ab2 | | TCCTGCAAGGCTTCTGGATACTCATTCACT | ATCTCCTGCAGAGCCAGCGAAAGTGTTGAT |
| | | GACTACAACATGCACTGGGTGAAACAGAGC | TATTATGGCTTTAGTTTTGTGAACTGGTTC |
| | | CGTGGAAAGAGCCTTGAGTGGATTGGATAT | CAACAGAAACCAGGACAGCCACCCAAACTC |
| | | ATTAACCCTAACAATGGTGTTCCCACGTAT | CTCATCTATAGTGCGTCCTACAAAGGATCC |
| | | AAGCAGAAGTTCAAGGGCAGGGCCACCTTG | GGGGTCCCTGTCAGGTTCAGTGGCAGTGGG |
| | | ACTGTAAACCAGTCCTCCAGCACAGCCTAC | TCTGGGACAGACTTCAGTCTCAGCATCCAT |
| | | ATGGAGATCCGCAGCCTGACATCGGAAGAT | CCTATGGAGGCGGATGATACTGCAATGTAT |
| | | TCTGCAGTCTATTACTGTACAAGAGGGGGT | TTCTGTCAGCAAAATAAGGAGGTTCCGCTC |
| | | GATCACCGGTTTGCTTACTGGGGCCAAGGG | ACGTTCGGTGCTGGGACCAAGCTGGAGCTG |
| | | ACTCTGGTCACTGTCTCTGCA | AAA |
| | | (SEQ ID NO: 278) | (SEQ ID NO: 279) |
| ACI-7087- | 4119E10D1 | CAGGTTCAACTGCAGCAGTCTGGGGCTGAG | GATGTCTTGATGACCCAAACTCCACTCTCC |
| 4119E10- | 2 | CTGGTGAGGCCTGGGGCTTCAGTGACGCTG | CTGCCTGTCAGTCTTGGAGATCAAGCCTCC |
| Ab2 | | TCCTGCAAGGCTTCGGGCTACACATTTTCT | ATCTCTTGCAGATCTAGTCAGAGCATTGTA |
| | | GACTATGAAATGAACTGGGTGAAGCAGACA | CATAGTAATGGAAACACCTATTTAGAATGG |
| | | CCTGTGCATGGCCTGGAATGGATTGGAGCT | TACCTGAAGAAAGCAGGCCAGTCTCCAAAG |
| | | ATTGATCCTGAAACTGGTGGTACTGCCTAC | GTCCTGATCTACAAAGTTTCCAACCGATTT |
| | | AATCAGAAGTTCAAGGGCAAGGCCATACTG | TCTGGGGTCCCAGACAGGTTCAGTGGCAGT |
| | | ACTTCAGACAAATCCTCCAGCACAGCCTAC | GGATCAGGGACAGATTTCACACTCAAAATC |
| | | ATGGAGCTCCGCAGCCTGACATCTGAGGAC | AGCAGGGTGGAGGCTGAGGATCTGGGAGTT |
| | | TCTGCCGTCTATTACTGTACAAGATTCCTG | TATTACTGCTTTCAAGGTTCACATGTTCCG |
| | | TTAATCGACTTTGACTATTGGGGCCAAGGC | TACACATTCGGAGGGGGGACCGAGCTGGAA |
| | | ACCACTCTCACAGTCTCCTCA | ATAAAA |
| | | SEQ ID NO: 308 | SEQ ID NO: 309 |
| ACI-7087- | 4125E6D5 | CAGGTCCAACTGCAGCAGCCTGGGACTGAA | GACATCCAGATGACCCAGTCTCCATCCTCC |
| 4125E6- | | CTGGTGAAGCCTGGGGCTTCAGTGAGGCTG | TTATCTGCCTCTCTGGGAGAAAGAGTCACT |
| Ab1 | | TCCTGCAAGGCTTCTGGCTACGCCTTCACC | CTCACTTGTCGGGCAAGTCAGGACATTGGT |
| | | AGCTACTGGATGCACTGGGTGAAGCAGAGG | AATTACTTAAACTGGCTTCAGCAGGAACCA |
| | | CCTGGACAAGGCCTTGAGTGGATTGGAAAT | GATGGAACTATTAAACGCCTGATCTACGCC |
| | | ATTAATCCTAGCAATGGTGGTACTAACTAC | ACATCCAGTTTAGATTCTGGTGTCCCCAAA |
| | | AATGAGAAGTTCAAGAACAAGGCCACACTG | AGGTTCAGTGGCAGTAGGTCTGGGTCAGAT |
| | | ACTGTAGACAAATCCTCCAGCACAGCCTAT | TATTCTCTCACCATCAGCAGCCTTGAGTCT |
| | | ATGCAGCTCAGCGGCCTGACATCTGAGGAC | GAAGATTTTGTAGACTATTACTGTCTACAA |
| | | TCTGCGGTCTATTATTGTGCAACGGGCCTT | TTTGCTAGTTCTCCGCTCACGTTCGGTCCT |
| | | CACTACTGGGGCCAAGGCACCACTCTCACA | GGGACCAAACTGGAACTGAAA |
| | | GTCTCCTCA | SEQ ID NO: 319 |
| | | SEQ ID NO: 318 | |
| ACI-7088- | 4301D5B10 | CAGGTCCAGCTGCAGCAGTCTGGACCTGAG | GACATTGTGCTGACCCAATCTCCAGCTTCT |
| 4301D5- | | CTGGTGAGGCCTGGGGCTTCAGTGAAGATA | TTGGCTGTGTCTCTAGGGCAGAGGGCCACC |
| Ab2 | | TCCTGCAAGGCTTCTGGCTACAGGTTCACA | ATCTCCTGCAGAGCCAGCGAAAGTGTTGAT |
| | | AGCTACTATATACACTGGGTGAAGCAGAGG | AATTATGGCATTAGTTTTATGAACTGGTTC |
| | | CCTGGACAGGGACTTGAGTGGATTGGATGG | CAACAGAAACCAGGACAGCCACCCAAACTC |
| | | ATTTATCCTGGAAGTGATAATACTAAGCAC | CTCATCTATGCTGCATCCAACCAAGGATCC |
| | | AATGACAAGTTCAAGGGCAAGGCCACACTG | GGGGTCCCTGCCAGGTTTAGTGGCATTGGG |
| | | ACGGCAGACACATCCTCCAGCACTGCCTAC | TCTGGGACAGACTTCAGCCTCAACATCCAT |
| | | ATGCAGCTCAGCAGCCTAACATCTGAGGAC | CCTATGGAGGAGGATGATACTGCAATGTAT |
| | | TCTGCGGTCTATTTCTGTGCAAGAGACTAC | TTCTGTCAGCAAAGTCAGGAGGTTCCGCTC |
| | | GACGTGGGGTTTGGTTACTGGGGCCAAGGG | ACGTTCGGTGCTGGGACCAAGCTGGAGCTG |
| | | ACTCTGGTCACTGTCTCTGCA | AAA |
| | | SEQ ID NO: 328 | SEQ ID NO: 329 |
| ACI-7088- | 4301E12B9 | GATGTACAGCTTCAGGAGTCAGGACCTGGC | GATGTTGTGTTGACCCAGACTCCACTCACT |
| 4301E12- | | CTCGTGAAACCTTCTCAGTCTCTGTCTCTC | TTGTCGGTTACCATTGGACAACCAGCCTCC |
| Ab2 | | ACCTGCTCTGTCACTGGCTACTCCATCACC | ATCTCTTGCAGGTCAAGTCAGAACCTCTTA |
| | | AGTGGTTATTACTGGAACTGGATCCGGCAG | GATAGTGATGGAGAGACATATTTGAATTGG |
| | | TTTCCAGGAAACAAACTGGAATGGATGGGC | TTGTTACAGAGGCCAGGCCAGTCTCCAAAG |
| | | TACATAAGCGACGATGGTAGTAAAAATTAC | CGCCTAATCTATCTGGTGTCTGAGCTGGAC |
| | | AACCCATCTCTCAAAAATCGAATCTCCATC | TCTGGAGTCCCTGACAGGTTCACTGGCAGT |
| | | ACTCGTGACACATCTAAGAACCAGCTTTTC | GGATCAGGGACAGATTTCACACTGAAAATC |
| | | ATGAAGTTGAATTCTGTGACTACTGAGGAC | AGCAGAGTGGAGGCTGAGGATTTGGGAGTT |
| | | ACAGCCACATATTACTGTGCAAGAGGCGAT | TATTATTGCTGGCAAGGTACACATTTTCCT |
| | | TCCCGCCTGGGCCAAGGGACTCTGGTCACT | CAGACGTTCGGTGGAGGCACCAAGCTGGAA |
| | | GTCTCTGCA | ATCATT |
| | | SEQ ID NO: 338 | SEQ ID NO: 339 |
| ACI-7088- | 4301H3A5 | GAGGTCCAGCTGCAACAATCTGGACCTGAA | GACATTGTGATGTCACAGTCTCCATCCTCC |
| 4301H3- | | CTGGTGAAGCCTGGGGCTTCAGTGAAGATA | CTGGCTGTGTCAGCAGGAGAGAAGGTCACT |
| Ab2 | | TCTTGTAAGGCTTCTGGATACACGTTCGCT | ATGAGCTGCAAATCCAGTCAGAGTCTCCTC |
| | | GACTACTTCATGAACTGGGTGAAGCAGAGC | AACAGTAGAACCCGAAAGAATTATTTGGCT |
| | | CATGGAAAGAGCCTTGAGTGGATTGGAGAT | TGGTACCAGCAGAAACCAGGGCAGTCTCCT |
| | | ATTAATCCTAACAATGGTGGTACTACCTAC | AAATTGTTGATCTACTCGGCATCCACTAGG |
| | | AACCAGAAGTTCAAGGGCAAGGCCACATTG | GAATCTGGGGTCCCTGATCGCTTCACAGGC |
| | | ACTGTAGACAAGTCCTCCAACACAGCCTAC | AGTGGATTTGGGACAGATTTCACTCTCACC |
| | | ATGGAGCTCCGCAGCCTGACATCTGAGGAC | ATCAGCAGTGTGCAGGCTGAGGACCTGGCA |
| | | TCTGCAGTCTACTACTGTGCAAGAGGTAGA | GTTTATTACTGCAAGCAATCTTATGATCTG |
| | | AACTACGCTATGGACTACTGGGGTCAAGGA | TGGACGTTCGGTGGAGGCACCAAGCTGGAA |
| | | ACCTCAGTCACCGTCTCCTCA | ATCAAA |
| | | SEQ ID NO: 348 | SEQ ID NO: 349 |
| ACI-7088- | 4303A1E7 | GAGGTTCAGCTGCAGCAGTCTGGGGCTGAA | GACATTGTGATGACCCAGTCTCAAAAATTC |
| 4303A1- | | CTTGTGAGGCCAGGGGCCTCAGTCAAGTTG | ATGTCCACATCAGTAGGAGACAGGGTCAGC |
| Ab1 | | TCCTGCACAGCTTCTGGCTTTAACATTAAA | ATCACCTGCAAGGCCAGTCAGAATGTGGGT |
| | | GACGACTATATGCACTGGGTGAAACAGAGG | ACTTCTGTAGGCTGGTATCAACAAAAAGCA |
| | | CCTGAACAGGGCCTGGAGTGGATTGGATGG | GGACAATCTCCTAAACTACTGATTCACTCG |
| | | ATTGATCCTGAGAATGGTGATTCTGAATAT | GCATCTAATCGGTACACTGGAGTCCCTGAT |
| | | GCCTCGAAGTTCCAGGGCAAGGCCACTATG | CGCTTCACAGGCAGTGGATCTGGGACAGAT |
| | | ACAGCAGACACATCCTCCAACACAGCCTAC | TTCACTCTCACCATCAACAATATGCAGTCT |
| | | CTGCAACTCAGCAGCCTGACATCTGAGGAC | GAAGACCTGGCAGATTATTTCTGCCAGCAA |
| | | ACTGCCGTCTATTATTGTAAAACATGGGGG | TATAGAAGTTATCCGCTCACGTTCGGTGCT |
| | | ACAGCTCAGGCCCTCTTTCCTTACTGGGGC | GGGACCAAGCTGGAGCTGAAA |
| | | CAAGGGACTCTGGTCACTGTCTCTGCA | SEQ ID NO: 359 |
| | | SEQ ID NO:358 | |
| ACI-7088- | 4303A3E4 | CAGGTTCAACTGCAGCAGTCTGGGGCTGAG | GATGTTTTGATGACCCAAACTCCACTCTCC |
| 4303A3- | | CTGGTGAGGCCTGGGGCTTCAGTGACGCTG | CTGCCTGTCAGTCTTGGAGATCAAGCCTCC |
| Ab1 | | TCCTGCAAGGCTTCGGGCTACACATTTACT | ATCTCTTGCAGATCTAGTCAGAGCATTGTA |
| | | GACTATGAAATGCACTGGGTGAAACAGACA | CATAGTAATGGAAACTCCTATTTAGAATGG |
| | | CCTGTGCATGGCCTGGAGTGGATTGGAGTT | TACCTGCAGAAACCAGGCCAGTCTCCAAAG |
| | | ATTGATCCTGAAACTGGTGGTGCTGTCCAG | CTCCTGATCTACAAAGTTTCCAACCGATTT |
| | | AATCAGAAGTTCAAGGGCAAGGCCATACTG | TCTGGGGTCCCAGACAGGTTCAGTGGCAGT |
| | | ACTGCAGACAATTCCTCCAGCACAGCCTAC | GGATCAGGGACAGATTTCACACTCAAGATC |
| | | ATGGACCTCCGCAGCCTGACATCTGAGGAC | AACAGAGTGGAGGCTGAGGATCTGGGAGTT |
| | | TCTGCCGTCTATAACTGTGCAATGGGTGCG | TATTACTGCTTTCAAGGTTCACATGTTCCA |
| | | GCATTACGGCTTGCTTACTGGGGCCAAGGG | TTCACGTTCGGCTCGGGGACAAAGTTGGAA |
| | | ACTCTGGTCACTGTCTCTGCA | ATAAAA |
| | | SEQ ID NO: 368 | SEQ ID NO: 369 |
| ACI-7088- | 4303B6C11 | GAGGTCCAGCTGCAACAATCTGGACCTGAG | GACATTGTGATGTCACAGTCTCCATCCTCC |
| 4303B6- | | CTGGTGAAGCCTGGGGCTTCAGTGAAGATA | CTGGCTGTGTCAGCACGAGAGAAGGTCACT |
| Ab2 | | TCCTGTAAGGCTTCTGGATACACGTTCACT | ATGAGCTGCAAATCCAGTCAGAGTCTGCTC |
| | | GACTACTACATGAACTGGGTGAAGCAGAGC | AACAGTAGAACCCGAAAGAACTACTTGGCT |
| | | CATGGAAAGAGCCTTGAGTGGATTGGAGAT | TGGTACCAGCAGAAACCAGGGCAGTCTCCT |
| | | ATTAATCCTAACAATGGTGGTACTACCTAC | AAACTGCTGATCTTCTGGGCTTCCACTAGG |
| | | AACCAGAAGTTCAAGGACAAGGCCACATTG | GAATCTGGGGTCCCTGATCGCTTCACTGGC |
| | | ACTGTGGACAGGTCCTCCAGCACAGCCTAC | AGTGGATCTGGGACAGATTTCACTCTCACC |
| | | ATGGAACTCCGCAGCCTGACATCTGGGGAC | ATCAGCAGTGTGCAGGCTGAAGACCTGGCA |
| | | TCTGCAGTCTATTACTGTGCAAGATCGGGG | GTTTATTACTGCAAACAATCTTATGATCTG |
| | | TACTCCGGTAGTCGCCTCTACTATGCTATG | TGGACGTTCGGTGGCGGCACCAAGCTGGAA |
| | | GACTACTGGAGTCAAGGATCCTCAGTCACC | ATCAAA |
| | | GTCTCCTCA | SEQ ID NO: 379 |
| | | SEQ ID NO: 378 | |
| ACI-7088- | 4303H6D7 | GAGGTTCAGCTGCAGCAGTCTGGGGCTGAG | GACATTTTGATGACCCAGTCTCAAAAATTC |
| 4303H6- | | CTTGTGAGGCCAGGGGCCTCAGTCAAGTTG | ATGTCCACATCAGTAGGAGACAGGGTCAGC |
| Ab1 | | TCCTGCACAGCTTCTGGCTTTAACATTCAA | GTCACCTGCAAGGCCAGTCAGAATGTGGGT |
| | | GACGACTATATGCACTGGGTGAAGCAGAGG | ACTAATGTAGCCTGGTATCAACAGAAACCA |
| | | CCTGAACAGGGCCTGGAGTGGATTGGTTGG | GGGCAATCTCCTAAACCACTGATTTCCTCG |
| | | ATTGATCCTGAGAATGGTGATACTGAATAT | GCATCCTCCCGGTACAGTGGCGTCCCTGAT |
| | | GCCTCGAAATTCCAGGGCAAGGCCACTTTA | CGCTTCACAGGCAGTGGATCTGGGACAGAT |
| | | ACAGCAGACACATCCTCCAACACAGCCTAC | TTCACTCTCACCATCAGCAATGTGCAGTCT |
| | | CTGCAGCTCAGCAGACTGACATCTGAGGAC | GAAGACTTGGCAGACTATTTCTGTCAGCAA |
| | | ACTGCCGTCTATTACTGTACTACAGCGGGC | TATAACCGCTATCCTCTCACGTTCGGTGCT |
| | | TCAGGCGTCCAACTCTTTGACTACTGGGGC | GGGACCAAGCTGGAGCTGAAA |
| | | CAAGGCACCACTCTCACAGTCTCCTCA | SEQ ID NO: 389 |
| | | SEQ ID NO: 388 | |
| ACI-7088- | 4305H7A4 | GAGGTTCAGCTGCAGCAGTCTGGGGCTGAA | GACATTGTGATGACCCAGTCTCAAAAATTC |
| 4305H7- | | CTTGTGAGGCCAGGGGCCTCAGTCAAGTTG | ATGTCCACATCAGTAGGAGACAGGGTCAGC |
| Ab1 | | TCCTGCACAGCTTCTGGCTTTAACATTAAA | ATCACCTGCAAGGCCAGTCAGAATGTGGGT |
| | | GACGACTATATGCACTGGGTGAAGCAGAGG | ACTGCTGTAGGCTGGTATCAACAAAAAGCA |
| | | CCTGAACAGGGCCTGGAGTGGATTGGATGG | GGACAATCTCCTAAACTACTGATTCACTCG |
| | | ATTGATCCTGAGAATGGTGATACTGAATAT | GCATCCAATCGGTACACTGGAGTCCCTGAT |
| | | GCCTCGAAGTTCCAGGGCAAGGCCACTATG | CGCTTCACAGGCAGTGGATCTGGGACAGAT |
| | | ATAGCAGACACATCCTCCAACACAGCCTAC | TTCACTCTCACCATCAACAATATGCAGTCT |
| | | CTGCAACTCAGCAGCCTGACATCTGAGGAC | GAAGACCTGGCAGATTATTTCTGCCAGCAA |
| | | ACTGCCGTCTATTATTGTAAAACATGGGGG | TATAGAAGTTATCCGCTCACGTTCGGTGCT |
| | | ACAACTCAGGCCCTCTTTCCTTACTGGGGC | GGGACCAAGCTGGAGCTGAAA |
| | | CAAGGGACTCTGGTCACTGTCTCTGCA | SEQ ID NO: 399 |
| | | SEQ ID NO: 398 | |
| ACI-7088- | 4317A4D2 | GAGGTTCAGCTGCAGCAGTCTGGGGCTGAA | GACATTGTGATGACCCAGTCTCAAAAGTTC |
| 4317A4- | | CTTGTGAGGCCAGGGGCCTCAGTCAAGTTG | ATGTACACATCAGTGGGAGACAGGGTCAGC |
| Ab1 | | TCCTGCACAGCTTCTGGCTTTAACATTAAA | ATCACCTGCAAGGCCAGTCAGAATGTGGGT |
| | | GACGACTATATGCACTGGGTGAAGCAGAGG | AATGCTGTAGGCTGGTATCAACAAAAAGCA |
| | | CCTGAACAGGGCCTGGAGTGGATTGGATGG | GGACAATCTCCTAAACTACTGATTCACTCG |
| | | ATTGATCCTGAGAATGGTGATACTGAATAT | GCATCCAATCGGTACACTGGAGTCCCTGAT |
| | | GCCTCGAAGTTCCAGGGCAAGGCCACTATG | CGCTTCACAGGCACTGGATCTGGGACAGAT |
| | | ACAGCAGACACATCCTCCAACACAGCCTAC | TTCACTCTCACCATCAACAATATGCAGTCT |
| | | CTGCAACTCAGCAGCCTGACATCTGAGGAC | GAAGACCTGGCAGATTATTTCTGCCAGCAA |
| | | ACTGCCGTCTATTATTGTAAAACATGGGGG | TATAGAAGTTATCCGCTCACGTTCGGTGCT |
| | | ACAACTCAGGCCCTCTTTCCTTACTGGGGC | GGGACCAAGCTGGAGCTGAAA |
| | | CAAGGGACTCTGGTCACTGTCTCTGCA | SEQ ID NO: 409 |
| | | SEQ ID NO: 408 | |
| ACI-7089- | 4409F1A8 | GATGTACAGCTTCAGGAGTCAGGACCTGGC | GATGTTGTGATGACCCAGACTCCACTCACT |
| 4409F1- | | CTCGTGAAACCTTCTCAGTCTCTGTCTCTC | TTGTCGGTTACCATTGGACAACCAGCCTCC |
| Ab1 | | ACCTGCTCTGTCACTGGCTACTCCATCACC | ATCTCTTGCAAGTCAAGTCAGAGCCTCTTA |
| | | AGGGGTTATTACTGGAACTGGATCCGGCAG | GATAGTGATGGAGAGACATATTTGAATTGG |
| | | TTTCCAGGAAACAAACTGGAATGGATGGGC | TTGTTACAGAGGCCAGGCCAGTCTCCAAAG |
| | | TACATAAGCTACGATGGTAGCAATAACTAC | CGCCTAATCTATCTGGTGTCTAAACTGGAC |
| | | AACCCATCTCTCAGAAATCGAATCTCCATC | TCTGGAGTCCCTGACAGGTTCACTGGTAGT |
| | | ACTCGTGACACATCTAAGAACCAGTTTTTC | GGATCAGGGACAGATTTCACACTGAAAATC |
| | | CTGAAGTTGAAATCTGTGACTACTGAGGAC | AGCAGAGTGGAGGCTGAGGATTTGGGAGTT |
| | | ACAGCCACATATTTCTGTGCAAGAGGGGAT | TATTATTGCTGGCAAGGTACACATTTTCCT |
| | | AGTAACTGGGGCCAAGGCACCACTCTCACA | CAGACGTTCGGTGGAGGCACCAAGTTGGAA |
| | | GTCTCCTCA | ATCAAA |
| | | SEQ ID NO: 418 | SEQ ID NO: 419 |
| ACI-7089- | 4415G5A1 | CAGGTTCAGCTGCAGCAGTCTGGAGCTGAG | GATATTGTGATAACCCAGGATGACCTCTCC |
| 4415G5- | 1 | CTGGCGAGGCCTGGGGCTTCAGTGAAGGTG | AATCCTGTCACTTCTGGAGAATCAGTTTCC |
| Ab1 | | TCCTGCAAGGCTTCTGGCTACACCTTCACA | ATCTCCTGCAGGTCTAGTAAGAGTCTCCTA |
| | | AGCTCTGGTATAAGCTGGTTGAAGCACAGA | TATAAGGATGGGAAGACATACTTGAATTGG |
| | | ACTGGACAGGGCCTTGAGTGGATTGGAGAC | TTTCTGCAGAGACCAGGACAATCTCCTCAG |
| | | ATTTATCCTAGAAGTGGTAATACTTACTAC | CTCCTGATCTATTTGATGTCCACCCGTGCA |
| | | AATGAGAAATTCAAGGACAAGGCCACACTG | TCAGGAGTCTCAGACCGGTTTAGTGGCAGT |
| | | ACTGCAGACAAATCCTCCAGCACGGCGTAC | GGGTCAGGAACAGATTTCACCCTGGAAATC |
| | | ATGGAGCTCCGCAGCCTGACATCTGAGGAC | AGTAGAGTGAAGGCTGAGGATGTGGGTGTG |
| | | TCTGCGGTCTATTTCTGTGCAAGTGGTAAC | TATTACTGTCAACAACTTTTAGAGTATCCG |
| | | TACTGGGGCCAAGGCACCACTCTCACAGTC | CTCACGTTCGGTGCTGGGACCAAGCTGGAG |
| | | TCCTCA | CTGAAA |
| | | SEQ ID NO: 428 | SEQ ID NO: 429 |
| ACI-7089- | 4417G6B1 | CAGGTTCAACTGCAGCAGTCTGGGGCTGAG | GATGTTGTGATGACCCAAACTCCACTCTCC |
| 4417G6- | 2 | TTGGTGAGGCCTGGGGCTTCAGTGACGCTG | CTGCCTGTCAGTCTTGGAGATCAAGCCTCC |
| Ab1 | | TCCTGCAAGGCTTCGGGCTACACATTTACT | ATCTCTTGCAGATCTAGTCAGAGCCTTCTA |
| | | GGCTATGAAATGCACTGGGTGAAGCAGACA | CACAGTAATGGATTCACCTATTTACATTGG |
| | | CCTGTGCATGGCCTGGAATGGATTGGAGCT | TACCTGCAGAAGCCAGGCCAGTCTCCAAAG |
| | | ATTGATCCTGAAACCGGTGGAACTGCCTAT | CTCCTGATCTACAGAGTTTCCAATCGATTT |
| | | ATTCAGAAGTTCAAGGGCAAGGCCACACTG | TCTGGGGTCCCAGACAGGTTCAGTGGCAGT |
| | | ACTGCAGACAAATCCTCCAGCACAGCCTAC | GGATCAGGGACAGATTTCACACTCAAGATC |
| | | ATGGAGCTCCGCAGCCTGACATCTGAGGAC | AGCAGAGTGGAGGCTGAGGATCTGGGAGTT |
| | | TCTGCCGTCTATTACTGTACAAGAGGCTGG | TATTTCTGCTCTCAAAGTACACATGTTCCG |
| | | GACTATTTTGACTACTGGGGCCAAGGCACC | TACACGTTCGGAGGGGGGACCAAGCTGGAA |
| | | ACTCTCACAGTCTCCTCA | ATAAAA |
| | | SEQ ID NO: 438 | SEQ ID NO: 439 |
| ACI-7089- | 4418C5G1 | GATGGACAACTTCAGGAGTCAGGACCTGGC | GATGTTGTGATGACCCAGACTCCACTCACT |
| 4418C5- | | CTCGTGAAACCTTCTCAGTCTCTGTCTCTC | TTGTCGGTTACCATTGGACAACCAGCCTCC |
| Ab1 | | ACCTGCTCTGTCACTGGCTACTCCATCACC | ATCTCTTGCAAGTCAAGTCAGAGCCTCTTA |
| | | AGTGGATATTACTGGAACTGGATCCGGCAG | GATAGTGATGGAGAGACATATTTGAATTGG |
| | | TTTCCAGGAAACAAACTGGAATGGATGGGC | TTATTACAGAGGCCAGGCCAGTCTCCAAAG |
| | | TACATAAACTACGATGGTAGCAATAACTAC | CGCCTAATCTATCTGGTGTCTAAACTGGAC |
| | | AACCCATCTCTCAAAAATCGAATCTCCATC | TCTGGAGTCCCTGACAGGTTCACTGGCAGT |
| | | ACTCGTGACACATCTAAGAATCAGTTTTTC | GGATCAGGGACAGATTTCACACTGAAAATC |
| | | CTGAAGTTCAATTTTGTGACTACTGAGGAC | AGCAGAGTGGAGGCTGAGGATTTGGGAGTT |
| | | ACAGCCACATATTACTGTGTGAGGGGGGAC | TATTATTGCTGGCAAGGTACACATTTTCCT |
| | | GTCTACTGGGGCCAAGGCACCACTCTCACA | CAGACGTTCGGTGGAGGCACCAAGCTGGAA |
| | | GTCTCCTCA | ATCAAA |
| | | SEQ ID NO: 448 | SEQ ID NO: 449 |
| ACI-7089- | 4418F6G7 | CAGGTTCAGCTGCAGCAGTCTGGAGCTGAG | GATATTGTGATAACCCAGGATGACCTCTCC |
| 4418F6- | | CTGGCGAGGCCTGGGGCTTCAGTGAAGGTG | AATCCTGTCACTTCTGGAGAATCAGTTTCC |
| Ab1 | | TCCTGCAAGGCTTCTGGCTACACCTTCACA | ATCTCCTGTAGGTCTAGTAAGAGTCTCCTA |
| | | AGTTCTGGTATAAGCTGGTTGAAGCACAGA | TATAAGGATGGGAAGACATACTTGAATTGG |
| | | ACTGGACAGGGCCTTGAGTGGATTGGAGAC | TTTCTGCAGAGACCAGGACAATCTCCTCAG |
| | | ATTTATCCTAGAAGTGGTAATACTTACTAC | CTCCTGATCTATTTGATGTCCACCCGTGCA |
| | | AATGAGAAATTCAAGGACAAGGCCACACTG | TCAGGAGTCTCAGACCGGTTTAGTGGCAGT |
| | | ACTGCAGACAAATCCTCCAGCACGGCGTAC | GGGTCAGGAACAGATTTCACCCTGGAAATC |
| | | ATGGAGCTCCGCAGCCTGACATCTGAGGAC | AGTAGAGTGAAGGCTGAGGATGTGGGTGTG |
| | | TCTGCGGTCTATTTCTGTTCAAGTGGTAAC | TATTACTGTCAACAACTTTTAGAGTATCCG |
| | | TACTGGGGCCAAGGCACCACTCTCACAGTC | CTCACGTTCGGTGCTGGGACCAAGCTGGAG |
| | | TCCTCA | CTGAAA |
| | | SEQ ID NO: 458 | SEQ ID NO: 459 |
| ACI-8033- | 917.5A12A | CAGGTCCACCTGAAGCAGTCTGGGGCTGAC | GATGTTGTGATGACCCAAACTCCACTCTCC |
| 5A 12-Ab1 | 11C9 | CTGGTGAGGCCTGGGGCTTCAGTGAAGCTG | CTGCCTGTCAGTCTTGGAGATCAAGCCTCC |
| | | TCCTGCAAGGCGTCTGGCTACACTTTCACT | ATCTCTTGTAGATCTAGTCAGAGCCTTGTA |
| | | GACTACTATATAAACTGGGTGAAGCAGAGG | CACAGTAATGGAAAAACCCATTTACATTGG |
| | | CCTGGACAGGGACTTGAGTGGATTGCAAGG | TACCTGCAGAAGCCAGGCCAGTCTCCAAAG |
| | | ATTTATCCTGGAAGTGGTAATACTTACTAC | CTCCTGATCTATAAAGTTTCCAACCGATTT |
| | | AATGAGAAGTTCAAGGGCAGGGCCACACTG | TCTGGGGTCCCAGACAGGTTCAGTGGCAGT |
| | | AGTGCAGAAAAATCCTCCACCACTGCCTAC | GGATCAGGGACAGATTTCACACTCAAGATC |
| | | ATGCAGCTCAGCAGCCTGACATCTGAGGAC | AGCAGAGTGGAGGCTGAGGATCTGGGAGTT |
| | | TCTGCTGTCTATTTCTGTGTAGTGGGGTAC | TATTTCTGCTCTCAAAGTACACATGTTCCG |
| | | TACGGTGCCTGGGGCCAAGGCACCACTCTC | TGGACGTTCGGTGGAGGCACCAAGCTGGAA |
| | | ACAGTCTCCTCA | ATCAAA |
| | | SEQ ID NO: 468 | SEQ ID NO: 469 |
| ACI-8033- | 917.25A3E | GAGGTGCAGCTTGTTGAGTCTGGTGGAGGA | GACATCAAGATGACCCAGTCTCCATCTTCC |
| 25A3-Ab1 | 9F6 | TTGGTGCAGCCTAAAGGGTCATTGAAACTC | ATGTATGCATCTCTAGGAGAGAGAGTCACT |
| | | TCATGTGCAGCCTCTGGATTCAGCTTCAAT | ATCACTTGCAAGGCGAGTCAGGACATTAAT |
| | | ACCTACGCCATGAACTGGGTCCGCCAGGCT | AGCTATTTAAGCTGGTTCCAGCAGAAACCA |
| | | CCAGGAAAGGGTTTGGAATGGGTTGCTCGC | GGGAAATCTCCTAAGACCCTAATCTATCGT |
| | | ATAAGAAGTAAAAGTAATAATTTTGCAACA | GCAAAAAGATTGGTAGATGGGGTCCCATCA |
| | | TATTATGCCGATTCAGTGAAAGACAGATTC | AGGTTCAGTGGCAGTGGATCTGGGCAAGAT |
| | | ACCATCTCCAGAGATGAATCAGAAAGCATG | TATTCTCTCACCATCAGCAGCCTGGAGTAT |
| | | CTCTATCTGCAAATGAACAACTTGAAAACT | GAAGATATGGGAATTTATTATTGTCTACAG |
| | | GAGGACACAGCCATGTATTACTGTGTGAGG | TATGATGAGTTTCCATTCACGTTCGGCTCG |
| | | TCCTTTGACTACTGGGGCCAAGGCACCACT | GGGACAAAGTTGGAAATAAAA |
| | | CTCACAGTCTCCTCA | SEQ ID NO: 479 |
| | | SEQ ID NO: 478 | |
| ACI-8033- | 917.1G10A | GATGTGCAGCTTCAGGAGTCAGGACCTGGC | GATGTTGTGATGACCCAAACTCCACTCTCC |
| 1G10-Ab1 | 10F6 | CTGGTGAAACCTTCTCAGACAGTGTTCCTC | CTGCCTGTCAGTCTTGGAGATCAAGCCTCC |
| | | ACCTGCACTGTCACTGGCATTTCCATCACC | ATCTCTTGCAGATCTAGTCAGAGCCTTGTA |
| | | ACTGGAAATTACAGGTGGAGCTGGATCCGG | CACAGTAATGGAAACACCTATTTACATTGG |
| | | CAGTTTCCAGGAAACAAACTGGAGTGGATA | TACCTGCAGAAGCCAGGCCAGTCTCCAAAG |
| | | GGGTACATATACTACAGTGGTACCATTACC | CTCCTGATCTACAAAGTTTCCAACCGATTT |
| | | TACAATCCATCTCTCACAAGTCGAACCACC | TCTGGGGTCCCAGACAGGTTCAGTGGCAGT |
| | | ATCACTAGAGACACTCCCAAGAACCAGTTC | GGATCAGGGACAGATTTCACACTCAAGATC |
| | | TTCCTGGAAATGAACTCTTTGACTGCTGAG | AGCAGAGTGGAGGCTGAGGATCTGGGAGTT |
| | | GACACAGCCACATACTACTGTGCACGGATT | TATTTCTGCTCTCAAAGTACACATGTTCCT |
| | | TACTACGGTAATGCTATGGACTACTGGGGT | CACACGTTCGGAGGGGGGACCAAGCTGGAA |
| | | CAAGGAACCTCAGTCACCGTCTCCTCA | ATAAAA |
| | | SEQ ID NO: 488 | SEQ ID NO: 489 |
| ACI-8033- | 917.19A2E | GAGGTGCAACTTGTTGAGTCTGGTGGAGGA | GATGTTGTGATGACCCAAACTCCACTCTCC |
| 19A2-Ab1 | 9E5 | TTGGTGCAGCCTAAAGGGTCATTGAAACTC | CTGCCTGTCAGTCTTGGAGATCAAGCCTCC |
| | | TCATGTGCAGCCTCTGGATTCAGCTTCAAT | ATCTCTTGCAGATCTAGTCAGAGCCTTGTA |
| | | ACCTACGCCATGAACTGGGTCCGCCAGGCT | CACAGTAATGGAAACACCTATTTATATTGG |
| | | CCAGGAAAGGGTTTGGAATGGGTTGCTCGC | TACCTGCAGAAGCCAGGCCAGTCTCCAAAG |
| | | ATAAGAAGTAAAAGTAATAATTATGCAACA | CTCCTGATCTACAAAGTTTCCAACCGACTT |
| | | TATTATGTCGATTCAGTGAAAGACAGATTC | TCTGGGGTCCCAGACAGGTTCAGTGGCAGT |
| | | ACCATCTCCAGAGATGATTCAGAAAGCATG | GGATCAGGGACAGATTTCACACTCAAGATC |
| | | CTCTATCTGCAAATGAACAACTTGAAAACT | AGCAGAGTGGAGGCTGAGGATCTGGGAGTT |
| | | GAGGACACAGCCCTGTATTACTGTGTGAGC | TATTTCTGCTCTCAAAGTACACATGTTCCA |
| | | GAATCCGCTTACTGGGGCCAAGGGACTCTG | TTCACGTTCGGCTCGGGGACAAAGTTGGAA |
| | | GTCACTGTCTCTGCA | ATAAAA |
| | | SEQ ID NO: 498 | SEQ ID NO: 499 |
| ACI-8033- | 917.8C10C | GATGTACAGCTTCAGGAGTCAGGACCTGGC | GATGTTGTGTTGACCCAGACTCCACTCACT |
| 8C10-Ab1 | 6G3 | CTCGTGAAACCTTCTCAGTCTCTGTCTCTC | TTGTCAGTTACCATTGGGCAACCAGCCTCC |
| | | ACCTGCTCTGTCACTGGCCAATCCATCACC | ATCTCTTGCAAGTCAAGTCAGAGCCTCTTA |
| | | AGTGGTTATTACTGGAACTGGATCCGGCAA | GATAGTGATGGAGAGACATATTTGAATTGG |
| | | TTTCCAGGAAACAAACTGGAATGGATGGGC | TTGTTACAGAGGCCAGGCCAGTCTCCAAAG |
| | | TACATAAGCAACGATGGTAGCAGTAAAACC | CGCCTAATCTATCTGGTGTCTGAACTGGAC |
| | | AACCCATCTCTCACAAATCGAATCTCCGTC | TCTGGAGTCTCTGACAGGTTCACTGGCAGT |
| | | ACTCGTGACACATCTAAGAACCAGGTTTTC | GGTTCAGGGACAGATTTCACACTGAAAATC |
| | | CTGAAGTTGAAATCTGTGACTACTGAGGAC | AGCAGACTGGAGGCTGAGGATTTGGGAGTT |
| | | ACAGCCACATATTACTGTGTAAGAGGGGAC | TATTATTGCTGGCAAGGTACACATTTTCCT |
| | | CAGCACTGGGGCCAAGGCACCGCTCTCACA | CAGACGTTCGGTGGAGGCACCAAGCTGGAA |
| | | GTCTCCTCA | ATCAAA |
| | | SEQ ID NO: 508 | SEQ ID NO: 509 |
| ACI-8033- | 917.7A2B6 | CAGGTCCAACTACAGCAGCCTGGGACTGAA | GACATTGTGATGTCACAGTCTCCATCCTCC |
| 7A2-Ab1 | A9 | CTGGTGAAGCCTGGGGCTTCAGTGAACCTG | CTAGCTGTGTCAGTTGGAGAGAAGGTTACT |
| | | CCCTGCAAGGCTTCTGGCTACACCTTCACC | ATGACCTGCAAGTCCAGTCAGAGCCTTTTA |
| | | AGCTACTGGATGCACTGGGTGAAGCAGAGG | TATAGAAGCAATCAAAAGAACTACTTGGCC |
| | | CCTGGTCAAGGCCTTGATTGGATTGGAAAT | TGGTACCAGCAGAAACCAGGACAGTCTCCT |
| | | GTTAATCCTAACAATAGTGATAGTAATTAC | AAACTGTTGATTTACTGGGCATTCACTAGG |
| | | AATGAGAAGTTCAAGAGGAAGGCCACACTG | GAATCTGGGGTCCCTGATCGCTTCACAGGC |
| | | ACTGTAGACAAATCCTCCAGCACAGCCTAC | AGTGGATCTGGGACAGATTTCACTCTCACC |
| | | ATGCACCTCAGCAGCCTGACATCTGAGGAC | ATCAGCAGTGTGAAGGCTGAAGACCTGGCA |
| | | TCTGCGGTCTATTATTGTGCAAGATCTCCT | GTTTATTACTGTCAGCAATATTATAGCTAT |
| | | TACTACGGTGGCCGTTACCTTGACTACTGG | CCTCTCACGTTCGGTGCTGGGACCAAGCTG |
| | | GGCCAAGGCACCACTCTCACAGTCTCCTCA | GAGCTGAAA |
| | | SEQ ID NO: 518 | SEQ ID NO: 519 |
| ACI-8033- | 917.1A12C | CAGGTCCACCTGAAGCAGTCTGGGGCTGAC | GATGTTGTGATGACCCAAACTCCACTCTCC |
| 1A12-Ab1 | 1B4 | CTGGTGAGGCCTGGGGCTTCAGTGAAGCTG | CTGCCTGTCAGTCTTGGAGATCAAGCCTCC |
| | | TCCTGCAAGGCGTCTGGCTACAGTTTCACT | ATCTCTTGCAGATCTAGTCAGAGCCTTGTA |
| | | GACTTTTATATAAATTGGGTGAAGCAGACG | CACAGTAATGGAAACACCCATTTGCATTGG |
| | | CCTGGACAGGGACTTGAGTGGATTGCGAGG | TACCTGCAGAAGCCAGGCCAGTCTCCAAAG |
| | | ATTTATCCTGGAAATAATAATACTTTCTAC | CTCCTGATCTATAAAGTTTCCAACCGATTT |
| | | AATGAGAAATTCAAGGGCAAGGCCACACTG | TCTGGGGTCCCAGACAGGTTCAGTGGCAGT |
| | | AGTGCAGAAAAATCCTCCACCACTGCCTAC | GGATCAGGGACAGATTTCACACTCAAGATC |
| | | ATGCAGCTCAGCAGCCTGACATCTGAGGAC | AGCAGAGTGGAGGCTGAGGATCTAGGATTT |
| | | TCTGCTGTCTATTTCTGTGTAGTGGGGTAC | TATTTCTGCTCTCAAAGTACACATGTTCCG |
| | | TACGGTGCCTGGGGCCAAGGCACCACTCTC | TGGACGTTCGGTGGAGGCACCAAGCTGGAA |
| | | ACAGTCTCCTCA | ATCAAA |
| | | SEQ ID NO: 528 | SEQ ID NO: 529 |
| ACI-8033- | 917.4F3F4 | GAGGTCCAGCTGCAACAATCTGGACCTGAG | GACATTGTGATGTCACAGTCTCCATCCTCC |
| 4F3-Ab1 | G6 | CTGGTGAAGCCTGGGGCTTCAGTGAAGATA | CTGGCTGTGTCAGCAGGAGAGAAGGTCACT |
| | | TCCTGTAAGGCTTCTGGATACACGTTCACT | ATGAGCTGCAAATCCAGTCAGAGTCTGCTC |
| | | GACTACTACATGAACTGGGTGAAGCAGAGC | AACAGTAGAACCCGAAAGAACTACTTGGCT |
| | | CATGGAAAGAGCCTTGAATGGATTGGAGAT | TGGTACCAGCAGAAACCAGGGCAGTCTCCT |
| | | ATTAATCCTAACACTGGTACTAATAGCTAC | AAACTGCTGATCTACTGGGCATCCACTAGG |
| | | AACCAGAAGTTCAAGGGCAGGGCCTCACTG | GAATCTGGGGTCCCTGATCGCTTCACAGGC |
| | | ACTGTAGACAAGTTCTCCAGCGCAGCCTAC | AGTGGATCTGGGACAGATTTCACTCTCACC |
| | | ATGGAGCTCCGCAGCCTGACATCTGAGGAC | ATCAGCAGTGTGCAGGCTGAAGACCTGGCA |
| | | TCTGCAGTCTATTACTGTGCAAGAACCGGC | GTTTATTACTGCAAGCAATCTTATAATCTG |
| | | TATGGCGACCCTATTTCCTCATATTACTAT | TGGACGTTCGGTGGAGGCACCAAGCTGGAA |
| | | GCTCTGGACTACTGGGGTCAAGGAACCTCA | ATCAAA |
| | | GTCACCGTCTCCTCA | SEQ ID NO: 539 |
| | | SEQ ID NO: 538 | |
| ACI-8033- | 917.17F5F | GAGGTCCAACTGCAACAATCTGGACCTGAG | GACATTGTGATGTCACAGTCTCCATCCTCC |
| 17F5-Ab1 | 5G9 | CTGGTGAAGCCTGGGGCTTCAGTGAAGATA | CTGGCTGTGTCAGCAGGAGAGAAGGTCACT |
| | | TCCTGTAAGGCTTCTGGATACACGTTCACT | ATGAGCTGCAAATCCAGTCAGAGTCTGCTC |
| | | GACTACTTCATGAACTGGGTGAAGCAGAGC | AACAGTAGAACCCGAAAGAACTACTTGGCT |
| | | CATGGAAAGAGCCTTGAGTGGATTGGAGAT | TGGTACCAGCAGAAACCAGGGCAGTCTCCT |
| | | ATTAATCCTAACATTGATGTTACTAACTAC | AAACTGCTGATCTACTGGGCATCCACTAGG |
| | | AACCAGAAGTTCAAGGGCAAGGCCACATTG | GAATCTGGGGTCCCTGATCGCTTCACAGGC |
| | | ACTGTAGACAAGTCCTCCAGCACAGCCTAC | AGTGGATCTGGGACAGATTTCACCCTCACC |
| | | ATGGAGCTCCGCAGCCTGACATCTGAGGAC | ATCAGCAGTGTGCAGGCTGAAGACCTGGCA |
| | | TCTGCAGTCTATTACTGTGCAAGAGGGCGG | GTTTATTACTGCAAGCAATCTTATGATCTG |
| | | GACTATGCTATGGACTTCTGGGGTCAAGGA | TGGACGTTCGGTGGAGGCACCAAGCTGGAA |
| | | ACCTCAGTCACCGTCTCCTCA | ATCAAA |
| | | SEQ ID NO: 548 | SEQ ID NO: 549 |
| ACI-8033- | 917.18C11 | CAGGTCCAACTCCAGCAGCCTGGGGCTGAG | GATGTTTTGATGACCCAAACTCCACTGTCC |
| 18C11- | A11F10 | CTTGTGAAGCCTGGGGCTTCAGTGAAGATG | CTGCCTGTCAGTCTTGGAGATCAAGCCTCC |
| Ab1 | | TCCTGCAAGGCTGCTGGCTACACCTTCAGC | ATCTCTTGCAGATCTAGTCAGAACATTGTA |
| | | AGCTACTGGATAACCTGGGTGAGGCAGAGG | CATAATAATGGAAACACCTATTTAGAATGG |
| | | CCTGGACAAGGCCTTGACTGGATTGGAGAT | TACCTGCAGAAACCAGGCCAGTCTCCAAAG |
| | | ATTTATCCTGGTGGAGGTGTTACTAACTAC | CTCCTGATCTACAAAGTTTCCAACCGATTT |
| | | AATGAGAAGTTCAAGACCAAGGCCACACTG | TCTGGGGTCCCAGACAGGTTCAGTGGCAGT |
| | | ACTGTAGACACATCCTCCAGCACAGCCTAC | GGATCAGGGACAGATTTCACACTCAAGATC |
| | | ATGCAGCTCAGCAGCCTGACATCTGAGGAC | AGCAGAGTGGAGGCTGAGGATCTGGGAGTT |
| | | TCTGCGGTCTATTACTGTGCGACAGCTCAG | TATTACTGCTTTCAAGGTTCACATGTTCCT |
| | | ACTACGTTTGCTTACTGGGGCCAAGGGACT | CGGACGTTCGGTGGAGGCACCAAGCTGGAA |
| | | CTGGTCACTGTCTCTGCA | ATCAAA |
| | | SEQ ID NO: 558 | SEQ ID NO: 559 |
| ACI-8033- | 917.18D12 | CAGGTCCAACTCCAGCAGCCTGGGGCTGAG | GATGTTTTGATGACCCAAACTCCACTCTCC |
| 18D12- | F10D6 | CTTGTGAAGCCTGGGGCTTCAGTGAAGATG | CTGCCCGTCAGTCTTGGAGATCAAGCCTCC |
| Ab1 | | TCCTGCAAGGCTTCTGGCTACACCTTCACC | ATCTCTTGCAGATCTAGTCAGAATATTGCA |
| | | AGCTACTGGATAACCTGGGTGAGGCAGAGG | CATAATAATGGAAACACCTATTTAGAATGG |
| | | CCTGGACAAGGCCTTGACTGGATTGGAGAT | TACCTGCAGAAACCAGGCCAGTCTCCAAAG |
| | | ATTTATCCTGGTGGAGGTGTTACTAACTAC | CTCCTGATCTACAAAGTTTCCAACCGATTT |
| | | AATGAGAAGTTCAAGACCAAGGCCACACTG | TCTGGGGTCCCAGACAGGTTCAGTGGCAGT |
| | | ACTGTAGACACATCCTCCAGCACAGCCTAC | GGATCAGGGACAGATTTCACACTCAAGATC |
| | | ATGCACCTCAGCAGCCTGACATCTGAGGAC | AGCAGAGTGGAGGCTGAGGATCTGGGAGTT |
| | | TCTGCGGTCTATTTCTGTGCGACAGCTCAG | TATTACTGCTTTCAAGGTTCACATGTTCCT |
| | | ACTACGTTTGCTCACTGGGGCCAAGGGACT | CGGACGTTCGGTGGAGGCACCAAGCTGGAA |
| | | CTGGTCACTGTCTCTGCA | ATCAAA |
| | | SEQ ID NO: 568 | SEQ ID NO: 569 |
| ACI-8033- | 917.1F8D8 | GATGTACAGCTTCAGGAGTCAGGACCTGGC | GATGTTGTGATGACCCAGACTCCACTCACT |
| 1F8-Ab1 | E4 | CTCGTGAAACCTTCTCAGTCTCTGTCTCTC | TTGTCGGTCACCATTGGACAACCAGCCTCC |
| | | ACCTGCTCTGTCACTGGCTACTCCATCACC | ATCTCTTGCAAGTCAAGTCAGAGCCTCTTA |
| | | AGTGGGTTTTACTGGAACTGGATCCGGCAA | GATAGTGATGGAGAGACATATTTGAATTGG |
| | | TTTCCAGGAAATAAACTGGAATGGATGGGC | TTGTTTCAGAGGCCAGGCCAGTCTCCAAAG |
| | | TACATAAGCTACGATGGTAGCAATAACTAC | CGCCTAATCTATCTGGTGTCTAAACTGGAC |
| | | AACCCATCTCTCAAAAATCGAATCTCCATT | TCTGGAGTCCCTGACAGGTTCACTGGCAGT |
| | | ATTCGTGACACATCTAAGAACCAGTTTTTC | GGATCAGGGACAGATTTCACACTGAAAATC |
| | | CTGAAGTTGAAATCTGTGACTTCTGAGGAC | AGCAGAGTGGAGCCTGAGGATTTGGGAGTT |
| | | ACAGCCACATATTATTGTGTAAGAGGGGAC | TATTATTGCTGGCAAGGTACACATTTTCCT |
| | | GTCGACTGGGGCCAAGGCACCACTCTCACT | CAGACGCTCGGTGGAGGCACCAAGCTGGAA |
| | | GTCTCCTCA | ATCAAA |
| | | SEQ ID NO: 578 | SEQ ID NO: 579 |
| ACI-8033- | 917.22E5C | GAGGTGCAACTAGTGGAGTCTGGGGGAGAC | GAAATTGTGCTCACCCAGTCTCCAGCACTC |
| 22E5-Ab1 | 5F7 | TTAGTGAAGCCTGGAGGGTCCCTGAAACTC | ATGGCTGCATCTCCAGGGGAGAAGGTCACC |
| | | TCCTGTGCAGCCTCTGGATTCACTTTCAGT | ATCACCTGCAGTGTCAGCTCAAGTATAAGT |
| | | AGCTATGGCATGTCTTGGGTTCGCCAGACT | TCCAGCAAGTTGCACTGGTACCAGCAGAAG |
| | | CCAGACAAGAGGCTGGAGTGGGTCGCAACC | TCAGAAACCTCCCCCAAACTCTGGATTTAT |
| | | ATTAGTAATGGTGGTAGTTACACCTACTAT | GGCACATCCAACCTGGCTTCTGGAGTCCCT |
| | | CCAGACAGTGTGAAGGGGCGATTCACCATC | GTTCGCTTCAGTGGCAGTGGATCTGGGACC |
| | | TCCAGAGACAATGCCAAGAACACCCTGTAC | TCTTATTCTCTCACAATCAGCAGCATGGAG |
| | | CTGCAAATGAGCAGTCTGAAGTCTGAGGAC | GCTGAAGATGCTGCCACTTATTACTGTCAA |
| | | ACAGCCATGTATTACTGTGCAAGACAATTA | CAGTGGAGTAGTTACCCACTCACGTTCGGT |
| | | CGACGGGACGGTTGGTACTTCGATGTCTGG | GCTGGGACCAAGCTGGAGCTGAAA |
| | | GGCACAGGGACCACGGTCACCGTCTCCTCA | SEQ ID NO: 589 |
| | | SEQ ID NO: 588 | |
| ACI-8033- | 917.27D8E | GAGGTGCAGCTTGTTGAGTCTGGTGGAGGA | GACATCAAGATGACCCAGTCTCCATCTTCC |
| 27D8-Ab1 | 1H10E10 | TTGGTGCAGCCTAAAGGGTCATTGAAACTC | ATGTATGCATCTCTAGGAGAGAGAGTCACT |
| | | TCATGTGCAGCCTCTGGATTCACCTTCAAT | ATCACTTGCAAGGCGAGTCAGGACATTAAT |
| | | ACCTACGCCATGAACTGGGTCCGCCAGGCT | AGCTATTTAAGCTGGTTCCAGCAGAAACCA |
| | | CCAGGAAAGGGTTTGGAATGGGTTGCTCGC | GGGAAATCTCCTAAGACCCTAATCTATCGT |
| | | ATAAGAAGTAAAAGTAATAATTTTGCAACA | GCAAAAAGATTGGTAGATGGGGTCCCATCA |
| | | TATTATGCCGATTCAGTGAAAGACAGATTC | AGGTTCAGTGGCAGTGGATCTGGGCAAGAT |
| | | ACCATCTCCAGAGATGAATCAGAAAGCATG | TATTCTCTCACCATCAGCAGCCTGGAGTAT |
| | | CTCTATCTGCAAATGAACAACTTGAAAGCT | GAAGATATGGGAATTTATTATTGTCTACAG |
| | | GAGGACACAGCCATGTATTACTGTGTGAGG | TATGATGAGTTTCCATTCACGTTCGGCTCG |
| | | TCCTTTGACTACTGGGGCCAAGGCACCACT | GGGACAAAGTTGGAAATAAAA |
| | | CTCACAGTCTCCTCA | SEQ ID NO: 479 |
| | | SEQ ID NO: 598 | |
| ACI-8033- | 917.21C8E | CAGGTCCAACTCCAGCAGCCTGGGGCTGAG | GATGTTTTGATGACCCAAACTCCACTCTCC |
| 21C8-Ab1 | 4C8 | CTTGTGAAGCCTGGGGCTTCAGTGAAGATG | CTGCCTGTCAGTCTTGGAGATCAAGCCTCC |
| | | TCCTGCAAGGCTTCTGGCTACACCTTCACC | ATCTCTTGCAGATCTAGTCAGAACATTGTA |
| | | AGCTACTGGATAACCTGGGTGAGGCAGAGG | CATAATAATGGAAACACCTATTTAGAATGG |
| | | CCTGGACAAGGCCTTGACTGGATTGGAGAT | TACCTGCAGAAACCAGGCCAGTCTCCAAAG |
| | | ATTTATCCTGGTGGAGGTGTTACTAACTAC | CTCCTGATCTACAAAGTTTCCAACCGATTT |
| | | AATGAGAAGTTCAAGACCAAGGCCACACTG | TCTGGGGTCCCAGACAGGTTCAGTGGCAGT |
| | | ACTGTAGACACATCCTCCAGCACAGCCTAC | GGATCAGGGACAGATTTCACACTCAAGATC |
| | | ATGCACCTCAGCAGCCTGACATCTGAGGAC | AGCAGAGTGGAGGCTGAGGATCTGGGAGTT |
| | | TCTGCGGTCTATTTCTGTGCGACAGCTCAG | TATTACTGCTTTCAAGGTTCACATGTTCCT |
| | | ACTACGTTTGCTTACTGGGGCCAAGGGACT | CGGACGTTCGGTGGAGGCACCAAGCTGGAA |
| | | CTGGTCACTGTCTCTGCA | ATCAAA |
| | | SEQ ID NO: 608 | SEQ ID NO: 609 |
| ACI-7079- | 3101E3C9 | CAGGTGCAGCTGAAGGAGTCAGGACCTGGC | GATGTTTTGATGACCCAAACTCCACTCTCC |
| 3101E3- | | CTGGTGGCGCCCTCACAGAGCCTGTCCATC | CTGCCTGTCAGTCTTGGAGATCAAGCCTCC |
| Ab1 | | ACATGCACTGTCTCTGGCTTCTCATTAACC | ATCTCTTGCAGATCTAGTCAGACCATTGTA |
| | | AGGTATATTATAAATTGGGTTCGCCAGCCA | CATAGTAATGGAAACACCTATTTAGAATGG |
| | | CCAGGAAAGGGTCTGGAGTGGCTTGGAGTA | TACCTGCAGAAACCAGGCCAGTCTCCAAAG |
| | | ATCTGGACTGGTGGAGGCACAGATTATAAT | CTCCTGATCTACAAAGTTTCCAACCGATTT |
| | | TCAGCTCTCAAATCCAGACTGAGCATCAGC | TCTGGGGTCCCAGACAGGTTCAGTGGCAGT |
| | | AAAGACAACTCCAAGAGTCAAGTTTTCTTA | GGATCAGGGACAGATTTCACACTCAAGATC |
| | | AAAATGAACAGTCTGCAAACTGATGACACA | AGCAGAGTGGAGGCTGAGGATCTGGGAGTT |
| | | GCCAGGTACTACTGTGCCAGAGAGTATGGT | TATTACTGCTTTCAAGGTTCACATGTTCCG |
| | | TACGACGGGGCCTGGTTTGCTTACTGGGGC | TACACGTTCGGAGGGGGGACCAAGCTGGAA |
| | | CAAGGGACTCTTGTCACTGTCTCTGCA | ATAAAA |
| | | SEQ ID NO: 618 | SEQ ID NO: 619 |
| ACI-7079- | 3103D9C9 | CAGGTTTCTCTGAAAGAGTCTGGCCCTGGG | GACATTGTGATGACACAGTCTCCATCCTCC |
| 3103D9- | | ATATTGCAGCCCTCCCAGACCCTCAGTCTG | CTGACTATGTCACTAGGACAGACGGTCACT |
| Ab1 | | ACTTGCTCTTTCTCTGGGTTTTCACTGAGC | ATGAGCTGCAAGTCCAGTCAGAGCCTTTTA |
| | | ACTTTTGGTATGGGTGTAGGCTGGATTCGT | AATAGTAGCAATCAAAAGAACCATCTGGCC |
| | | CAGCCTTCAGGGAAGGGTCTGGACTGGCTG | TGGTACCAGCAGAGACCAGGACAGTCTCCC |
| | | ACACACATTTGGTGGGATGATGATAAGTAC | AAACTTCTGGTATACTTTGCATCCACTAGG |
| | | TATAATCCAGGCCTGAAGAGTCGCCTCACA | GAATCTGGGGTCCCTGAGCGCTTCATAGGC |
| | | ATCTCCAAGGATACCTCCAAAAACCAGGTA | GGTGGATCTGGGACAGATTTCACTCTTACC |
| | | TTCCTCGAGATCGCCAATGTGGACACTGCA | ATCAGCAATGTGCAGTCTGAAGACCTGACA |
| | | GATACTGCCACATATTACTGTGCGCGAATA | GATTACTTCTGTCAGCAACATTATAGCACT |
| | | GGAGATTACTACGGTACTAGAGGGTACTTC | CCGTACACGTTCGGAGGGGGGACCAAGCTG |
| | | GATGTCTGGGGCACAGGGACCACGGTCACC | GAAATAAAA |
| | | GTCTCCTCA | SEQ ID NO: 629 |
| | | SEQ ID NO: 628 | |
| ACI-7079- | 3103G12D | GATGTGCAGCTTCAGGAGTCAGGACCTGGC | GATGTTTTGATGACCCAAGCTCCACTCTCC |
| 3103G12- | 2B10 | ATGGTGAAACCTTCTCAGTCACTTTCCCTC | CTGCCTGTCAGTCTTGGAGATCAAGCCTCC |
| Ab2 | | ACCTGCACTGTCACTGGCTACTCCATCACC | ATCTCGTGCAGATCTAGTCAGAGCATTGTA |
| | | AGTGGTTATGACTGGCACTGGATCCGACAT | CATAGTAATGGAGACACCTATTTAGAATGG |
| | | TTTCCAGGAAACAAACTGGAGTGGATGGGC | TACCTGCAGAAACCAGGCCAGTCTCCAAAG |
| | | TACATAACCTACAGTGGTAGCACTAACTAC | CTCCTGATCTACAAAGTTTCCAACCGATTT |
| | | AACCCATCCCTCAAAAGTCGAATCTCCATC | TCTGGGGTCCCAGACAGGTTCAGTGGCAGT |
| | | ACTCATGACACATCTAAGAACCATTTCTTC | GGATCAGGGACAGATTTCACACTCAAGATC |
| | | CTGAAGTTGACTTCTGTGACTACTGAGGAC | AGCAGAGTGGAGGCTGAGGATCTGGGAGTT |
| | | ACAGCCACATATTACTGTGCAAGAGACACG | TATTACTGCTTTCAAGGTTCACATGTTCCT |
| | | GTAGACGCCTGGTTTGCTTACTGGGGCCAG | CCGACGTTCGGTGGAGGCACCAAGCTGGAA |
| | | GGGACTCTGGTCACTGTCTCTGCA | ATCAAA |
| | | SEQ ID NO: 638 | SEQ ID NO: 639 |
| ACI-7079- | 3104F12F2 | CAGGTTACTCTGAAAGAGTCTGGCCCTGGA | GACATTGTGATGACACAGTCTCCATCCTCC |
| 3104F12- | H7 | ATAGTGCAGCCCTCCCAGACCCTCAGTCTG | CTGGCTATGTCAGTTGGACAGAAGGTCACT |
| Ab2 | | ACTTGCTCTTTCTCTGGATTTTCACTGAAC | TTGAGGTGCAAGTCCAGTCAGAGCCTTTTA |
| | | ACTTTTGGTTTGGGTGTAGGCTGGATTCGT | AATAGTAGTAATCAAAAGAACCATTTGGCC |
| | | CAGCCTTCAGGGAAGGGTCTGGACTGGCTG | TGGTACCAGCAGAAACCAGGACAGTCTCCT |
| | | ACACACATTTGGTGGGATGATGATAAGTAC | AAACTTCTGCTATGCTTTGCATCCACTAGG |
| | | TATAATCCAGCCCTGAAGAGTCGGCTCACA | GAATCTGGGGTCCCTGATCGCTTCATAGGC |
| | | ATCTCCAAGGATACCTCCAAAAACCAGGTA | AGTGGATCTGGGACAGATTTCACTCTTACC |
| | | TTCCTCAAGATCGCCAATGTGGACACTGCA | ATCAGTAGTGTGCAGGCTGAAGACCTGGCA |
| | | GATACTGCCACATACTACTGTGCTCGAATA | GATTACTTCTGTCAGCAACATTATAGCACT |
| | | GGCGATTTCTACGGTAGTAGAGGATATTTC | CCGTACACGTTCGGAGGGGGGACCAAGCTG |
| | | GATGTCTGGGGCACAGGGACCACGGTCACC | GAAATAAAA |
| | | GTCTCCTCA | SEQ ID NO: 649 |
| | | SEQ ID NO: 648 | |
| ACI-7079- | 3106C5B7 | CAGGTTACTCTGAAAGAGTCTGGCCCTGGG | GACATTGTGATGACACAGTCTCCATCCTCC |
| 3106C5- | | ATATTGCAGCCCTCCCAGACCCTCAGTCTG | CTGGCTATGTCAGTAGGACAGAAGGTCACT |
| Ab1 | | ACTTGTTCTTTCTCTGGGTTTTCACTGAGT | ATGAGCTGCAAGTCCAGGCAGAGCCTTTTA |
| | | ACTTTTGGTATGGGTGTAGGCTGGATTCGT | AATAGTAGTAATCAAAAGAATCATTTGGCC |
| | | CAGCCTTCAGGGAAGGGTCTGGAGTGGCTA | TGGTACCAGCAGAAACCAGGACAGTCTCCT |
| | | ACACACATTTGGTGGGATGATGATAAGTAC | AAACTTCTGGTATACTTTGCATCCACTAGG |
| | | TATAACCCAGCCCTGAAGAGTCGGCTCACA | GAATCTGGGGTCCCTGATCGCTTCATAGGC |
| | | ATCTCCAGGGATCCCTCCAAAAACCAGGTA | AGTGGATCTGGGACAGATTTCACTCTTACC |
| | | TTCTCAAGATCGCCAATGTGGACACTGCAG | ATCAGCAGTGTGCAGGCTGAAGACCTGGCA |
| | | ATACTGCCACATACTACTGTGCTCGAATAG | GATTACTTCTGTCAGCAACATTATAGCACT |
| | | GGGATTACTACGGTAGTAGAGGATACTTCG | CCGTACACGTTCGGGGGGGGGACCAAGCTG |
| | | ATGTCTGGGGCACAGGGACCACGGTCACCG | GAAATAAAA |
| | | TCTCCTCA | SEQ ID NO: 659 |
| | | SEQ ID NO: 658 | |
| ACI-7079- | 3106F2C8 | GAGGTTCAGCTGCAGCAGTCTGGGGCTGAG | GACATTGTGATGACCCAGTCTCAAAAATTC |
| 3106F2- | | CTTGTGAGGCCAGGGGCCTCAGTCAAGTTG | ATGTCCACATCAGTAGGAGACAGGGTCAGC |
| Ab1 | | TCCTGCACAGCTTCTGGCTTTAACGTTAAA | GTCACCTGCAAGGCCAGTCAGAATGTGGGA |
| | | GACGACTATATGCACTGGGTGAAGCAGAGG | ACTAATGTAGCCTGGTATCAACAGAAAGTA |
| | | CCTGAACAGGGCCTGGAGTGGATTGGATGG | GGACAATCTCCTAAAGCACTGATTTACTCG |
| | | ATTGATCCTGAGAATGGTGATACTGAATAT | GCATCCTACCGGTACAGTGGAGTCCCTGAT |
| | | GCCTCGAAGTTCCAGGGCAAGGCCACTTTA | CGCTTCACAGGCAGTGGATCTGGGACAGAT |
| | | ACAGCAGACACATCCTCCAACACAGCCTAC | TTCACTCTCACCATCAGCAATGTGCAGTCT |
| | | CTGCAGCTCAGCGGCCTGACATCTGTGGAC | GAAGACTTGGCAGACTATTTCTGTCAGCAA |
| | | ACTGCCGTCTATTACTGTACTACAGCCGGT | TATAACAGCTATCCTCTCACGTTCGGCTCG |
| | | ACTAGCCCGTACTACTTTGACTACTGGGGC | GGGACAAAGTTGGAAATAAAA |
| | | CAAGGCACCACTCTCACAGTCTCCTCC | SEQ ID NO: 669 |
| | | SEQ ID NO: 668 | |
| ACI-7079- | 3112H1F3 | GAGGTCCAGCTGCAACAATCTGGACCTGAA | GACATTGTGATGTCACAGTCTCCATCCTCC |
| 3112H1- | | CTGGTGAAGCCTGGGGCTTCAGTGAAGATA | CTGGCTGTGTCAGCAGGAGAGAAGGTCACT |
| Ab1 | | TCCTGTAAGGCTTCTGGATACACGTTCAGT | ATGAGCTGCAAATCCAGTCAGAGTCTGCTC |
| | | GACTACTTCATGAACTGGGTGAAGCAGAGT | AACAGTAGAACCCGGAAGAACTACTTGGCT |
| | | CATGGAAAAAGCCTTGAGTGGATTGGAGAT | TGGTACCAGCAGAAACCAGGACAGTCTCCT |
| | | ATTAATCCTAACAATGGTGGTTCTAGCTAC | AAACTGCTGATCTACTGGGCATCCACTAGG |
| | | ATCCAGAAGTTCAAGGGCAAGGCCACATTG | GAATCTGGGGTCCCTGATCGCTTCACAGGC |
| | | ACTGTAGACAAGTCCTCCACCACAGCCTAT | AGTGGATCTGGGACAGATTTCACTCTCACC |
| | | ATGGAGCTCCGCAGCCTGACATCTGAGGAC | ATCAGCAGTGTGCAGGCTGAAGACCTGGCA |
| | | TCAGCAGTCTATTACTGTGCAAGAGGTAGC | GTTTATTACTGCAAGCAATCTTATGATCTG |
| | | AACTATGCTATGGACTCCTGGGGTCAGGGA | TGGACGTTCGGTGGGGGCACCAAGCTGGAA |
| | | ACCTCAGTCACCGTCTCCTCA | ATCAAA |
| | | SEQ ID NO: 678 | SEQ ID NO: 679 |
| ACI-7079- | 3107E6A3 | CAGGTTACTCTGAAAGAGTCTGGCCCTGGG | GACATTGTGATGACACAGTCTCCATCCTCC |
| 3107E6- | | ATATTGCAGCCCTCCCAGACCCTCAGTCTG | CTGGCCATGTCAGTAGGACAGAAGGTCACT |
| Ab1 | | ACTTGTTCTTTCTCTGGGTTTTCACTGAGC | ATGAGCTGCAAGTCCAGTCAGAGCCTTTTA |
| | | ACTTTTGGTATGGGTGTAGGCTGGATTCGT | AATAGTAGCAATCAAAAGAACCATTTGGCC |
| | | CAGCCTTCAGGGAAGGGTCTGGAGTGGCTG | TGGTATCAGCAGAAACCAGGACAGTCTCCT |
| | | ACACACATTTGGTGGGATGATGATAAATAC | AAACTTCTGGTATACTTTGCATCCACTAGG |
| | | TATAACCCAGCCCTTAAGAGTCGGCTCACA | GACTCTGGGGTCCCTGATCGCTTCATAGGC |
| | | ATCTCCAAGGATGCCTCCAAAAACCAGATG | AGTGGATCTGGGACAGATTTCACTCTTACC |
| | | TTCCTCAAGATCGCCAATGTGGACACTGCA | ATCAGCAGTGTGCAGGCTGAGGACCTGGCA |
| | | GATACTGCCACATACTTCTGTGCTCGAATA | GATTACTTCTGTCAGCAACATTATAGCACT |
| | | GGGGATTACTACGGTAGTAGAGGGTTCTTC | CCGTACACGTTCGGAGGGGGGACCAAGCTG |
| | | GATGTCTGGGGCACAGGGACCACGGTCACC | GAAATAAAA |
| | | GTCTCCTCA | SEQ ID NO: 689 |
| | | SEQ ID NO: 688 | |
| ACI-7079- | 3108C10G6 | GAACTGCAGCTGGTGGAGTCTGGGGGAGGC | GATGTTGTGATGACCCAGACTCCACGCACT |
| 3108C10- | | TTAGTGAGGCCTGGAGGGTCCCTGAAACTC | TTGTCGGTAATCATTGGACAACCAGCCTCC |
| Ab2 | | TCCTGTGCAACCTCTGGATTCACTTTCAGA | ATCTCTTGCAAGTCAAGTCAGAGCCTCTTA |
| | | AACTATGCCATGTCTTGGGTTCGCCAGACT | GATAGTGATGGAAAGACATACTTGAGTTGG |
| | | CCGGCAAAGAGGCTGGAGTGGGTCGCAACC | TTGTTACAGAGGCCAGGCCAGTCTCCAAAG |
| | | ATTAGTGATGGTGGTACTTACACCTTCTAT | CGCCTAATCTATCTGGTGTCTAAAGTGGAC |
| | | TCAGACAATGTAAAGGGCCGATTCACCATC | TCTGGAGTCCCTGACAGGTTCACTGGCAGT |
| | | TCCAGAGACAATGTCAAGAACACCTTGTAC | GGATCAGGGACAGATTTCACACTGAAAATC |
| | | CTTCAAATGAGCCATCTGAAGTCTGAGGAC | AGCAGAGTGGAGGCTGAGGATTTGGGAGTT |
| | | ACAGCCATGTATTACTGTTCAAGAGCCGGC | TATTATTGCTGGCAAGGTACATATTTTCCT |
| | | TCTGGCCACTGGGGCCAAGGCACCACTCTC | CGGACGTTCGGTGGAGGCACCAAGCTGGAA |
| | | ACAGTCTCCTCA | ATCAAA |
| | | SEQ ID NO: 698 | SEQ ID NO: 699 |
| ACI-8030- | 6106F5B10 | CAGGTCCAGCTGCAGCAGTCTGGACCTGAA | GACATTGTGCTGACCCAATCTCCAGCTTCT |
| 6106F5- | | CTGGTGAAGCCTGGGGCTTCAGTGAAGATA | TTGGCTGTGTCTCTAGGGCAGAGGGCCACC |
| Ab1 | | TCCTGCAAGGCTTCTGGCTACAGCTTCACA | ATCTCCTGCAAGGCCAGCCAAAGTGTTGAT |
| | | ACCTACTATATACACTGGATGAAGCAGAGG | TATGGTGGTGATAGTTGGCTGAACTGGTAC |
| | | CCTGGACAGGGACCTGAGTGGATTGGATGG | CAACAGAAACCAGGACAGCCACCCAAACTC |
| | | ATTTATCCTGGAAGGGGTTATACTAAGTAC | CTCATCTATGCTGCATCCAATCTAGATTCT |
| | | AATGAGAAGTTCAAGGACAAGGCCGCACAG | GGGATCCCAGCCAGGTTTGGTGGCAGTGGG |
| | | ACGGCAGACACATCCTCCAACACTGCCTAC | TCTGGGACAGACTTCACCCTCAACATCCAT |
| | | ATGCAGCTCAGCAGCCTAAGATCTGAGGAC | CCTGTGGAGGAGGAGGATGCTGCAACCTAT |
| | | TCTGCGGTCTATTACTGTGCAAGAGACTGG | TACTGTCAACAAAGTGGAGAGGATCCGTTC |
| | | GAAACCGGATTTCCTTACTGGGGCCAAGGT | ACGTTCGGAGGGGGGACCAATCTGGAAATA |
| | | ACTCTGGTCACTGTCTCTGCA | AAA |
| | | SEQ ID NO: 708 | SEQ ID NO: 709 |
| ACI-8031- | 6207G10E6 | CAGGTTCAGCTGCAGCAGTCTGGGGCTGAG | GACATCTTGCTGACTCAGTCTCCAGCCATC |
| 6207G10- | | CTGGTGAAGCCTGGGGCCTCAGTGAAGATT | CTGTCTGTGAGTCCAGGAGATAGAGTCAGT |
| Ab1 | | TCCTGCAAAGCTTCTGGCTACGCATTCAGT | TTCTCCTGCAGGGCCAGTCAGAGCATTGGC |
| | | AGCTACTGGATGAACTGGGTGAAGCAGAGG | ACAAGGATACACTGGTATCAGCAAAGAACA |
| | | CCTGGAAAGGGTCTTGAGTGGATTGGACAG | AATGGTTCTCCAAGGCTTCTCATAAAGTAT |
| | | ATTTATCCTGGAGATGGTGATACTAACTAC | GCTTCTGAGTCTATCTCTGGGACCCCTTCC |
| | | AACGGAAAGTTCAAGGGCAAGGCCACACTG | AGGTTTAGTGGCAGTGGATCAGGGACAGAT |
| | | ACTGCAGACAGATCCTCCAGCACAGCCTAC | TTTACTCTTAGCATCAACAGTGTGGAGTCT |
| | | ATGCAGCTCAGCAGCCTGACCTCTGAGGAC | GAAGATATTGCAGATTATTACTGTCAACAA |
| | | TCTGCGGTCTATTTCTGTGCAATTACGCAC | AGTAATAGTTGGCCACTCACGTTCGGTGCT |
| | | TATGACTACTGGGGCCAAGGCACCACTCTC | GGGACCAAGCTGGAGCTGAAA |
| | | ACAGTCTCCTCA | SEQ ID NO: 719 |
| | | SEQ ID NO: 718 | |
| ACI-8032- | 6301A10E12 | CAGGTTCAGCTGCAGCAGTCTGGAGCTGAA | GATGTTTTGATGACCCAAACTCCACTCTCC |
| 6301A10- | | CTGGCGAGGCCTGGGGCTTCAGTGAAGCTG | CTGCCTGTCAGTCTTGGAGATCAAGCCTCC |
| Ab2 | | TCCTGCAAGGCTTCTGGCTACACCTTCACA | ATCTCTTGCAGATCTAGTCACAGCATTGAA |
| | | AGCTATGGTATCAGCTGGGTGAAGCAGAGA | CATAGTAATGGAAACACCTATTTAGAATGG |
| | | ACTGGACAGGGCCTTGAGTGGATTGGAGAG | TGCCTGCAGAAACCAGGCCAGCCTCCAAAG |
| | | ATTTATCCTAGAAGTGGTAATACTTACTAC | TTCCTGATCTACAAAGTTTCCAACCGATTT |
| | | AATGAGAAGTTCAAGGGCAAGGCCACACTG | TCTGGGGTCCCAGACAGGTTCAGTGGCAGT |
| | | ACTGCAGACAAATCCTCCAGCACAGCGTAC | GGATCAGGGACAGATTTCACACTCAGGATC |
| | | ATGGAGCTCCGCAGCCTGACATCTGAGGAC | AGTAGAGTGGAGGCTGAGGATCTGGGAGTT |
| | | TCTGCGGTCTATTTCTGTGCAAGTGAGAGC | TATTACTGCTTTCAAGGTTCACATGTTCCT |
| | | TACTATGGTGACTACGACTGGTACTTCGAT | CCGACGTTCGGTGGAGGCACCAAGCTGGAA |
| | | GTCTGGGGCACTGGGACCACGGTCACCGTC | ATCAAA |
| | | TCCTCA | SEQ ID NO: 729 |
| | | SEQ ID NO: 728 | |
| ACI-8032- | 6301C8G12 | CAGGTTCAGCTGCAGCAGTCTGGAGCTGAA | GATGTTTTGATGACCCAAATTCCACTCTCC |
| 6301C8- | | CTGGCGAGGCCTGGGACTTCAGTGAAGCTG | CTGCCTGTCAGTCTTGGAGATCAAGCCTCC |
| Ab2 | | TCCTGCAAGGCTTCTGGCTACACCTTCACA | ATCTCTTGCAGATCTAGTCAGAGCATTGTA |
| | | ACCTATGGTATAAGCTGGGTGAAGCAGAGA | CATAGTAATGGAAACACCTATTTAGAATGG |
| | | ACTGGACAGGGCCTTGAGTGGATTGGAGAG | CACCTGCAGAAACCAGGCCAGTCTCCAAAG |
| | | ATTTATCCTAGAAGTGGTAATACTTACTAC | CTCCTGATCTACGAAGTTTCCAACCGATTT |
| | | AATGAGAAATTCAAGGGCAAGGCCACACTG | TCTGGGGTCCCAGACAGGTTCAGTGGCAGT |
| | | ACTGCAGACAAGCCCTCCAGCACAGCGTAC | GGATCAGGGACAGATTTCACACTCAATATC |
| | | ATGGAGCTCCGCAGCCTGACATCTGAGGAC | AGCAGAGTGGAGGCTGAGGATCTGGGAGTT |
| | | TCTGCGGTCTATTTCTGTGCAAGTGAGAGT | TATTACTGCTTTCAAGGTTCACATGTTCCT |
| | | TACTATGGTGACTACGCCTGGTACTTCGAT | CCGACGTTCGGTGGAGGCACCAAGCTGGAA |
| | | GTCTGGGGCACAGGGACCACGGTCACCGTC | ATCAAA |
| | | TCCTCA | SEQ ID NO: 739 |
| | | SEQ ID NO: 738 | |
| ACI-8032- | 6301G2D11 | GAGGTCCAACTGCAACAATCTGGACCTGAG | GACATTGTGATGTCACAGTCTCCATCCTCC |
| 6301G2- | | CTGGTGAAGCCTGGGGCTTCAGTGAAGATA | CTGGCTGTGTCAGCAGGAGAGAAGGTCACT |
| Ab2 | | TCCTGTAAGGCTTCTGGATACACGTTCACT | ATGAGCTGCAAATCCAGTCAGAGTCTGCTC |
| | | GACTACTTCATGAACTGGGTGAAGCAGAGC | AACAGTAGAACCCGAAAGAACTACTTGGCT |
| | | CATGGAAAGAGCCTTGAGTGGATTGGAGAT | TGGTACCAGCAGAAACCAGGGCAGTCTCCT |
| | | ATTAATCCTAACATTGGTGTTACTAACTAC | AAACTGCTGATCTACTGGGCATCCACTAGG |
| | | AACCCGAAGTTCAAGGACAGGGCCACATTG | GAATCTGGGGTCCCTGATCGCTTCACAGGC |
| | | ACTGTAGACAAGTCCTCCAGCTCAGCCTAC | AGTGGATCTGGGACAGATTTCACTCTCACC |
| | | ATGGAGCTCCGCAGCCTGACATCTGAGGAC | ATCAGCAGTGTGCAGGCTGAAGACCTGGCA |
| | | TCTACAGTCTATTACTGTGCAAGAGGGGGA | GTTTATTTCTGCAAGCAATCTTATGATCTG |
| | | GGGAGTAATCCCCTTTACTATGCTATGGAC | TGGACGTTCGGTGGAGGCACCAAGCTGGAA |
| | | TACTGGGGTCAAGGAACCTCAGTCACCGTC | ATCAAA |
| | | TCCTCA | SEQ ID NO: 749 |
| | | SEQ ID NO: 748 | |
| ACI-8032- | 6304F3D12 | CAGGTTCAGCTGCAGCAGTCTGGAGCTGAG | GATGTTTTGATGACCCAAATTCCACTCTCC |
| 6304F3- | | CTGGCGAGACCTGGGGCTTCAGTGAAGCTG | CTGCCTGTCAGTCCTGGAGATCAAGTTTCC |
| Ab1 | | TCCTGCAAGGCTTCTGGCTACACCTTCACA | ATCTCTTGCAGATCTAGTCAGAGCATTGTA |
| | | AGCTATGGTGTAAGCTGGGTGAAGCAGAGA | CATAGTAATGGAAACACCTATTTAGAATGG |
| | | ACTGGACAGGGCCTTGAGTGGATTGGAGAG | TACCTGCAGAAACCAGGCCAGTCTCCAAAG |
| | | ATTTATCCTAGAAGTGGTAATACTTACTAC | CCCCTGATCTACAAAGTTTCCAACCGATTT |
| | | AATGAGAAGTTCAAGGGCAAGGCCACACTG | TCTGGGGTCCCAGACAGGTTCAGTGGCAGT |
| | | ACTGCAGACAAATCCTCCAGCACAGCGTAC | GGATCAGGGACAGATTTCACACTCAAGATC |
| | | ATGGAGCTCCGCAGCCTGACATCTGAGGAC | AGCAGAGTGGAGGCTGAGGATCTGGGAGTT |
| | | TCTGCGGTCTATTTCTGTGCAAGTGAGAGC | TATTACTGCTTTCAAGGTTCACATGTTCCT |
| | | TACTATGGTGACTACGACTGGTATTTCGAT | CCGACGTTCGGTGGAGGCACCAAGCTGGAA |
| | | GTCTGGGGCACAGGGACCACGGTCACCGTC | ATCAAA |
| | | TCCTCA | SEQ ID NO: 759 |
| | | SEQ ID NO: 758 | |
| ACI-8032- | 6307F1H10 | CAGGTTCAGCTGCAGCAGTCTGGAGCTGAG | GATGTTTTGATGACCCAAACTCCACTCTCC |
| 6307F1- | | CTGGCGAGGCCTGGGGCTTCAGTGAAGCTG | CTGCCTGTCAGTCTTGGAGATCAAGCCTCC |
| Ab2 | | TCCTGCAAGGCTTCTGGCTACACCTTCACA | ATCTCTTGCAGATCTAGTCAGAGCGTTGTA |
| | | AGCTATGGTATGAGTTGGGTGAAACAGAGA | CATAGTAATGGAATCACCTATTTAGAATGG |
| | | ACTGGACAGGGCCTTGAGTGGATTGGAGAG | TACCTGCAGAAACCAGGCCAGTCTCCAAAG |
| | | ATTTATCCTAGAAGTGGTAATACTTACTAC | CTCCTGATCTACAAAGTTTCCAACCGATTT |
| | | AATGAGAAGTTCAAGGGCAAGGCCACACTG | TCTGGGGTCCCAGACAGGTTCAGTGGCAGT |
| | | ACTTCAGACAAATCCTCCAGCACATCATAC | GGATCAGGGACATATTTCACACTCAAGATC |
| | | ATGGAGCTCCGCAGCCTGACATCTGAGGAC | AGCAGAGTGGAGGCTGAGGATCTTGGAGTT |
| | | TCTGCGGTCTATTTCTGTGCAAGTGAGAGC | TATTACTGCTTTCAAGGTTCACATGTTCCT |
| | | TACTATGGTGACTACGCCTGGTACTTCGAT | CCGACGTTCGGTGGAGGCACCAAGCTGGAA |
| | | GTCTGGGGCACAGGGACCACGGTCACCGTC | ATCAAA |
| | | TCCTCA | SEQ ID NO: 769 |
| | | SEQ ID NO: 768 | |
| ACI-8032- | 6313G2A1 | GAGGTCCAGCTGCAACAATCTGGACCTGAG | GACATTGTGATGTCACAGTCGCCATCCTCC |
| 6313G2- | 0 | CTGGTGAAGCCTGGGGCTTCAGTGAAGATA | CTGGCTGTGTCAGCAGGAGAGAAGGTCACT |
| Ab1 | | TCCTGTAAGGCTTCTGGATACACGTTCACT | ATGAACTGCAAATCCAGTCAGAGTCTGCTC |
| | | GACTACTACATGAACTGGGTGAAGCAGAGC | AACAGTAGAACCCGAAAGAACTACTTGGCT |
| | | CATGGAAAGAGCCTTGAGTGGATTGGAGAT | TGGTACCAGCAGAAACCAGGGCAGTCTCCA |
| | | ATTGATCCTAACAATGGTGTTACTAGTTAC | AAACTACTGATCTACTGGGCATCCACTAGG |
| | | AGCCAGAAGTTCAAGGGCAAGGCCACATTG | GAATCTGGGGTCCCTGATCGCTTCATAGGC |
| | | ACTGTAGACAAGTCCTCCAGCACAGCCTAC | AGTGGATCTGGGACAGGTTTCACTCTCACC |
| | | ATGGAGCTCCGCAGCCTGACATCTGAAGAC | ATTAGCAGTGTGCAGGCTGAAGACCTGGCA |
| | | TCTGCAGTCTATTACTGTGCAAGGACGGGG | GTTTATTACTGCAAGCAATCTTATGATCTG |
| | | TACTACGGTACTTCTCTATACTATGCTATG | TGGACGTTCGGTGGAGGCACCAAGCTGGAA |
| | | GACTACTGGGGTCAAGGAACCTCAGTCACC | ATCAAA |
| | | GTCTCCTCA | SEQ ID NO: 779 |
| | | SEQ ID NO: 778 | |
| ACI-8032- | 6314A3E4 | CAGGTTCAGCTGCAGCAGTCTGGAGCTGAG | GATGTTTTGATGACCCAAATTCCACTCTCC |
| 6314A3- | | CTGGCGAGACCTGGGGCTTCAGTGAAGCTG | CTGCCTGTCAGTCCTGGAGATCAAGTTTTC |
| Ab3 | | TCCTGCAAGGCTTCTGGCTACACCTTCACA | ATCTCTTGCAGATCTAGTCAGAGCATTGTA |
| | | AGCTATGGTGTAAGCTGGGTGAAGCAGAGA | CATAGTAATGGAAACACCTATTTAGAATGG |
| | | ACTGGACAGGGCCTTGAGTGGATTGGAGAG | TACCTGCAGAAACCAGGCCAGTCTCCAAAG |
| | | ATTTATCCTAGAAGTGGTAATACTTACTAC | CCCCTGATCTACAAAGTTTCCAACCGATTT |
| | | AATGAGAAGTTCAAGGGCAAGGCCACACTG | TCTGGGGTCCCAGACAGGTTCAGTGGCAGT |
| | | ACTGCAGACAAATCCTCCAGCACAGCGTAC | GGATCAGGGACAGATTTCACACTCAAGATC |
| | | ATGGAGCTCCGCAGCCTGACATCTGAGGAC | AGCAGAGTGGAGGCTGAGGATCTGGGAGTT |
| | | TCTGCGGTCTATTTCTGTGCAAGTGAGAGC | TATTACTGCTTTCAAGGTTCACATGTTCCT |
| | | TACTATGGTGACTACGACTGGTATTTCGAT | CCGACGTTCGGTGGAGGCACCAAGCTGGAA |
| | | GTCTGGGGCACAGGGACCACGGTCACCGTC | ATCAAA |
| | | TCCTCA | SEQ ID NO: 789 |
| | | SEQ ID NO: 758 | |
| ACI-8033- | 6401F2E3 | CAGGTCCAACTGCAGCAGCCTGGGGCTGAG | GATGTTTTGATGACCCAAACTCCACTCTCC |
| 6401F2- | | CTTGTGAAGCCTGGGGCTTCAGTGAAGATG | CTGCCTGTCAGTCTTGGAGATCAAGCCTCC |
| Ab1 | | TCCTGCAAGGCTTCTGGCTACACCTTCACC | ATCTCTTGCAGATCTAGTCAGAGTGTTGTG |
| | | AACTACTGGATTACCTGGGTAAAACAGAGG | CATGTTAATGGAAACACCTATTTAGATTGG |
| | | CCTGGACAAGGCCTTGAGTGGATTGGGGAT | TACCTGCAGAAACCAGGCCAGTCTCCAAAG |
| | | ATTTATCCTGGTAATGGTGATGTTGACTAC | CTCCTGATCTACAAAGTTTCCAACCGATTT |
| | | AGTGAGATCTTCAAGAATAAGGCCAAAATG | TCTGGGGTCCCAGACAGGTTCAGTGGCAGT |
| | | ACTGTAGACACATCCTCCACCACAGCCTAC | GGATCAGGGACAGATTTCACGCTCAGGATC |
| | | ATGCAGCTCAGCAGCCTGACATCTGAGGAC | ACCAGAGTGGAGGCTGAGGATCTGGGAATT |
| | | TCTGCGGTCTATCACTGTGTCTTGGGTCAG | TATTACTGTTTTCAAGGTTCACATGTTCCT |
| | | ACTTCCTTTGGTCATTGGGGCCAAGGGACT | CGGACGTTCGGTGGAGGCACCAAGCTGGAA |
| | | CTGGTCACTGTCTCTGCA | ATCAAA |
| | | SEQ ID NO: 798 | SEQ ID NO: 799 |
| ACI-8033- | 6402E2E4 | GAGGTCCAGCTGCAACATTCTGGACCTGAC | GACATTGTGATGTCACAGTCTCCATCCTCC |
| 6402E2- | | CTGGTGAAGCCTGGGGCTTCAGTGAAGATA | CTGGCTGTGTCAGCAGGAGAGAGGGTCACT |
| Ab2 | | TCCTGTAAGGCTTCTGGATACACGTTCACT | ATGAGCTGCAAATCCAGTCAGAGTCTGTTC |
| | | GACTACTACATGAACTGGGTGAAACAGAGC | AACAGTAGAACCCGAAAGAACTACTTGGCT |
| | | CATGGAAAGAGCCTTGAGTGGATTGGAGAT | TGGTACCAGCAGAAACCAGGGCAGTCTCCT |
| | | ATTAATCCTAACAATGGTGATACTACCTAC | AAACTGCTGATCTACTGGGCATCCACTAGG |
| | | AACCAGAAGTTCAAGGGCAGGGCCACATTG | GAATCTGGGGTCCCTGATCGCTTCACAGGC |
| | | ACTGTAGACAAGTCCTCCAGCACAGCCTAC | AGTGGATCTGGGACAGATTTCAGTCTCAAC |
| | | ATGGAGCTCCGCAGCCTGACATCTGAGGAC | ATCAGCAGTGTGCAGGCTGAAGACCTGGCA |
| | | TCTGCAGTCTATTACTGTGCAAGATCGGGG | GTTTATTACTGCAAGCAATCTTATGATCTG |
| | | AGTAATTACTATGCTATGGACTCCTGGGGT | TGGACGTTCGGTGGAGGCACCAAGCTGGAA |
| | | CAAGGAACCTCAGTCACCGTCTCCTCA | ATCAAA |
| | | SEQ ID NO: 808 | SEQ ID NO: 809 |
| ACI-8033- | 6402E10B3 | GATGTACAGCTTCAGGAGTCAGGACCTGGC | GATGTTGTGATGACCCAGACTCCACTCACT |
| 6402E10- | | CTCGTGAAACCTGTTCAGTCTTTGTCTCTC | TTGTCGGTTACCATTGGACAACCAGCCTCC |
| Ab1 | | ACCTGCTCTGTCACTGGCTACTCCATCACC | ATCTCTTGCAAGTCAAGTCAGAGCCTCTTA |
| | | AGTGGTTATTACTGGAACTGGATCCGGCAG | GATAGTGATGGAGAGACATATTTGAATTGG |
| | | TTTCCAGGAAACAAACTGGAATGGATGGGC | TTGTTACAGAGGCCAGGCCAGTCTCCAAAG |
| | | TACATAAGCTCCGATGGTAGCAATAACTAC | CGCCTAATCTATCTGGTGTCTAAAGTGGAC |
| | | AACCCATCTCTCAAAAATCGAATCTCCATC | TCTGGAGTCCCTGACAGGTTCACTGGCAGA |
| | | TCTCGTGACACATCTAAGAACCAGTTTTTC | GGATCAGGGACAGATTTCACACTGAAAATC |
| | | CTGAAGTTGGATTCTGTGACTACTGAGGAC | AGCAGAGTGGAGGCTGAGGATTTGGGAGTT |
| | | ACAGCCACATATTACTGTGTAAGAGCGGAC | TATTATTGCTGGCAAGGCACACATTTTCCT |
| | | AACTACTGGGGCCAAGGCACCACTCTCACA | CAGACGTTCGGTGGAGGCACCAAGCTGGAA |
| | | GTCTCCTCG | ATCAAG |
| | | SEQ ID NO: 818 | SEQ ID NO: 819 |
| ACI-8033- | 6403A4A3 | GAGGTGCAGCTTGTTGAGTCTGGTGGAGGA | CAAATTGTTCTCACCCAGTCTCCAGCAATC |
| 6403A4- | | TTGGTGCAGCCTAAAGGGTCATTGAAACTC | ATGTCTGCATCTCTAGGGGAGGAGATCACC |
| Ab1 | | TCATGTGCAGCCTCTGGATTCAGTTTCAAT | CTAACCTGCAGTGCCAGTTCGAGTATAAGT |
| | | ACCTACGCCATGAACTGGGTCCGCCAGGCT | TACATGCACTGGTACCAGCAGAAGTCAGGC |
| | | CCAGGAAAGGGTTTGGAATGGGTTGCTCGC | ACTTCTCCCAAACTCTTGATTCATAGCACA |
| | | ATAAGAAGTAAAAGTAATAATTATACAACA | TTCAACCTGGCTTCTGGAGTCCCTTCTCGC |
| | | TATTATGCCGATTCAGTGAAAGACAGATTC | TTCAGTGGCAGTGGGTCTGGGACCTTTTAT |
| | | ACCATCTCCAGAGATGATTCAGAAAGCATG | TCTCTCACAATCAGCAGTGTGGAGGCTGAA |
| | | CTCTATCTGCAAATGAACAACTTGAAAACT | GATGCTGCCGATTATTACTGCCATCAGTGG |
| | | GAGGACACAGCCATGTATTACTGTGTGAGA | AGTAGTTATTACACGTTCGGAGGGGGGACC |
| | | ACTATGGACTACTGGGGTCAAGGAACCTCA | AAGCTGGAAATAAAA |
| | | GTCACCGTCTCCTCA | SEQ ID NO: 829 |
| | | SEQ ID NO: 828 | |
| ACI-8033- | 6403E11B4 | GATGTACAGCTTCAGGAGTCAGGACCTGGC | GATGTTGTGATGACCCAGACTCCACTCACT |
| 6403E11- | | CTCGTGAAACCTTCTCAGTCTCTGTCTCTC | TTGTCGGTCACCATTGGACAACCAGCCTCC |
| Ab2 | | ACCTGCTCTGTCACTGGCTACTCCATCACC | ATCTCTTGCAAGTCAAGTCAGAGCCTCTTA |
| | | AGTGCTTATTACTGGAACTGGATCCGGCAG | GATGGTGATGGAGAGACATATTTGAATTGG |
| | | TTTCCAGGAAACAAACTGGAATGGATGGGC | TTGTTACAGAGGCCAGGCCAGTCTCCAAAG |
| | | TACATAAGCTACGATGGTAGCAATAACTAC | CGCCTAATCTATCTGGTATCTAAACTGGAC |
| | | ATCCCTTCTCTCAAAAATCGAATCTCCATC | TCTGGAGTCCCTGACAGGTTCACTGGCAGT |
| | | AGTCGTGACACATCTAAGAACCAGTTTTTC | GGATCAGGGACAGATTTCACACTGAAAATC |
| | | CTGAAGTTGAATTCTGTGACTACTGAGGAC | AGCAGAGTGGAGGCTGAGGATTTGGGAGTT |
| | | ACAGCCACATATTACTGTATAAGAGGGGAT | TATTATTGCTGGCAAGGTACACATTTTCCT |
| | | GTTAACTGGGGCCAAGGCACCACTCTCACA | CAGACGTTCGGTGGAGGCACCAAGCTGGAA |
| | | GTCTCCTCA | ATCAAA |
| | | SEQ ID NO: 838 | SEQ ID NO: 839 |
| ACI-7067- | 4813-R4A- | GAGGTGCAGCTGGAGGAGTCTGGTGGAGGA | CAGGCTGTTGTGACTCAGGAATCTGCACTC |
| 4813-R4A- | G7 | TTGGTGCACCCTAAAGGATCATTGAAACTC | ACCACATCACCTGGTGAAACAGTCACACTC |
| G7-rec1 | | TCATGTGCCGCCTCTGGTTTCACCTTCAAT | ACTTGTCGCTCAAGTACTGGGGCTGTTACA |
| | | TCCTATGCCATGCACTGGGTCCGCCAGGCT | ACTAGTAACTATGCCAACTGGGTCCAAGAA |
| | | CCAGGAAAGGGTTTGGAATGGGTTGCTCGC | AAACCAGATCATTTATTCACTGGTCTAATA |
| | | ATAAGAAGTAAAAGAAGTAATTATGCAACA | GGTGGTACCAACAACCGAGCTCCAGGTGTT |
| | | TATTATGCCGATTCAGTGAAAGACAGATTC | CCTGCCAGATTCTCAGGCTCCCTGATTGGA |
| | | ACCATCTCCAGAGATGATTCACAAAGCATG | GACAAGGCTGCCCTCACCATCACAGGGGCA |
| | | GTCTATCTGGAAATGAACAACCTGGAAGCT | CAGACTGAGGATGAGGCAATATATTTCTGT |
| | | GAGGACACAGCCACGTATTACTGTGTGAGA | GCTCTATGGTACAGCAACCATTGGGTGTTC |
| | | GGGGGACGAGAGGCTATGGACTTCTGGGGT | GGTGGAGGAACCAAACTGACTGTCCTA |
| | | CAAGGAACCTCAGTCACCGTCTCCTCA | (SEQ ID NO: 849) |
| | | (SEQ ID NO: 848) | |

TABLE 7
Amino acid sequence of the heavy chain and light chain variable domains (VH and VL) and
their CDRs

| Anti- | | | | | | | | | |
|---|---|---|---|---|---|---|---|---|---|
| body | Hybridoma | | VH | VH | VH | | VL | VL | VL |
| Code | Code | VH | CDR1 | CDR2 | CDR3 | VL | CDR1 | CDR2 | CDR3 |
| ACI- | 1101C8F7 | EVQLVES | IYAMN | RIRSKSN | VGLRFYA | DVLMTQT | RSSQSIV | KVSNRFS | FQGSQGP |
| 7067- | | GGGLVQP | (SEQ ID | NYATYYA | MDY | PLSLPVS | HSNGNTY | (SEQ ID | LT |
| 1101C8- | | KGSLKLS | NO: 11) | DSVKD | (SEQ ID | LGDQASI | LE | NO: 16) | (SEQ ID |
| Ab2 | | CAASGFS | | (SEQ ID | NO: 13) | SCRSSQS | (SEQ ID | | NO: 17) |
| | | FNIYAMN | | NO: 12) | | IVHSNGN | NO: 15) | | |
| | | WVRQAPG | | | | TYLEWYL | | | |
| | | KGLEWVA | | | | QKPGQSP | | | |
| | | RIRSKSN | | | | KLLIYKV | | | |
| | | NYATYYA | | | | SNRFSGV | | | |
| | | DSVKDRF | | | | PDRFSGS | | | |
| | | TISRADS | | | | GSGTDFT | | | |
| | | ESMLYLQ | | | | LKISRVE | | | |
| | | MNNLKTE | | | | AEDLGVY | | | |
| | | DTAMYYC | | | | YCFQGSQ | | | |
| | | VRVGLRF | | | | GPLTFGA | | | |
| | | YAMDYWG | | | | GTKLELK | | | |
| | | QGTSVTV | | | | (SEQ ID | | | |
| | | SS | | | | NO: 14) | | | |
| | | (SEQ ID | | | | | | | |
| | | NO: 10) | | | | | | | |
| ACI- | 1102G3F2 | EVKLEES | DAWM | EIRNKAH | YSY | SIVMTQT | KASQSVT | STSNRYS | QQDYRIP |
| 7067- | | GGGLVQP | (SEQ ID | NHATYYA | | PKFLLVS | KDVA | (SEQ ID | YT |
| 1102G3- | | GGSMKLS | NO: 21) | ESVKG | | AGDRVTI | (SEQ ID | NO: 26) | (SEQ ID |
| Ab1 | | CAASGFT | | (SEQ ID | | TCKASQS | NO: 25) | | NO: 27) |
| | | FSDAWMN | | NO: 22) | | VTKDVAW | | | |
| | | WVRQSPE | | | | YQQKPGQ | | | |
| | | KGLEWVA | | | | SPKLLIY | | | |
| | | EIRNKAH | | | | STSNRYS | | | |
| | | NHATYYA | | | | GVPDRFT | | | |
| | | ESVKGRF | | | | GSGYGTD | | | |
| | | TISGDDS | | | | FTFTINT | | | |
| | | KSSVYLQ | | | | VQTEDLA | | | |
| | | MNNLRAE | | | | VYFCQQD | | | |
| | | DTGIYYC | | | | YRIPYTF | | | |
| | | TIYSYWG | | | | GGGTKLE | | | |
| | | QGTLVTV | | | | IK | | | |
| | | SA | | | | (SEQ ID | | | |
| | | (SEQ ID | | | | NO: 24) | | | |
| | | NO: 20) | | | | | | | |
| ACI- | 1106A8H3 | EVQLVES | TYAMH | RIRSKGS | GHGSSYF | QIVLTQS | SASSSVS | DTSNLAS | QQWNSHP |
| 7067- | | GGGLVQP | (SEQ ID | NYATNYA | SY | PAIMSAS | Y | (SEQ ID | PT |
| 1106A8- | | KGSLKLS | NO: 31) | DSVKD | (SEQ ID | PGEKVTM | (SEQ ID | NO: 36) | (SEQ ID |
| Ab2 | | CAASGFT | | (SEQ ID | NO: 33) | TCSASSS | NO: 35) | | NO: 37) |
| | | FNTYAMH | | NO: 32) | | VSYMHWY | | | |
| | | WVRQAPG | | | | QQKSGTS | | | |
| | | KGLEWVA | | | | PKRWIYD | | | |
| | | RIRSKGS | | | | TSNLASG | | | |
| | | NYATNYA | | | | VPARFSG | | | |
| | | DSVKDRF | | | | SGSGTSY | | | |
| | | TISRDDS | | | | SLTISSM | | | |
| | | QSMLYLQ | | | | EAEDAAT | | | |
| | | MNNLKTE | | | | YYCQQWN | | | |
| | | DTAMYYC | | | | SHPPTFG | | | |
| | | VRGHGSS | | | | AGTKLEL | | | |
| | | YFSYWGQ | | | | K | | | |
| | | GTLVTVS | | | | (SEQ ID | | | |
| | | A | | | | NO: 34) | | | |
| | | (SEQ ID | | | | | | | |
| | | NO: 30) | | | | | | | |
| ACI- | 1107G5B6 | QVQLQQP | KYWMH | NINPNNG | AMDY | DIQMTQS | RASQDIG | ATSSLDS | LQFGSSP |
| 7067- | | GTELVKP | (SEQ ID | DTNYNEK | (SEQ ID | PSSLSAF | NNLN | (SEQ ID | LT |
| 1107G5- | | GASVKLS | NO: 41) | FKS | NO: 43) | LGERVSL | (SEQ ID | NO: 46) | (SEQ ID |
| Ab2 | | CKASGYT | | (SEQ ID | | TCRASQD | NO: 45) | | NO: 47) |
| | | FTKYWMH | | NO: 42) | | IGNNLNW | | | |
| | | WVKQRPG | | | | FQQEPDG | | | |
| | | QGLEWIG | | | | TIKRLIY | | | |
| | | NINPNNG | | | | ATSSLDS | | | |
| | | DTNYNEK | | | | GVPKRFS | | | |
| | | FKSKATL | | | | GSRSGSE | | | |
| | | TVDKSSS | | | | YSLTISS | | | |
| | | TAYMQLS | | | | LESEDFV | | | |
| | | SLTSEDS | | | | DYYCLQF | | | |
| | | AVYYCAI | | | | GSSPLTF | | | |
| | | AMDYWGQ | | | | GAGTKLE | | | |
| | | GTSVTVS | | | | LK | | | |
| | | S | | | | (SEQ ID | | | |
| | | (SEQ ID | | | | NO: 44) | | | |
| | | NO: 40) | | | | | | | |
| ACI- | 1108H1E1 | EVKLVES | DAWM | EIRNKAH | YSF | SIVMTQT | KASQSVT | SASNRYS | QQDYRIP |
| 7067- | | GGGLVQP | (SEQ ID | NHATNYA | | PKFLLVS | NYVA | (SEQ ID | YT |
| 1108H1- | | GGSMKLS | NO: 21) | ESVKG | | AGDRVTI | (SEQ ID | NO: 56) | (SEQ ID |
| Ab1 | | CTASGFT | | (SEQ ID | | TCKASQS | NO: 55) | | NO: 27) |
| | | FSDAWMN | | NO: 52) | | VTNYVAW | | | |
| | | WVRQSPE | | | | YHQKPGQ | | | |
| | | KGLEWVA | | | | SPKLLIY | | | |
| | | EIRNKAH | | | | SASNRYS | | | |
| | | NHATNYA | | | | GVPDRFT | | | |
| | | ESVKGRF | | | | GSGYGTD | | | |
| | | TISGDDS | | | | FTFTINT | | | |
| | | KSSVYLQ | | | | VQTEDLA | | | |
| | | MNNLRAE | | | | VYFCQQD | | | |
| | | DTGIYYC | | | | YRIPYTF | | | |
| | | TIYSFWG | | | | GGGTKLE | | | |
| | | QGTLVTV | | | | IK | | | |
| | | SA | | | | (SEQ ID | | | |
| | | (SEQ ID | | | | NO: 54) | | | |
| | | NO: 50) | | | | | | | |
| ACI- | 1111B12H10 | QVQLLQP | TYWMH | NINPING | AMDY | DIQMTQS | RASQDIG | ATSSLDS | LQFASSP |
| 7067- | | GTALVMP | (SEQ ID | GSNYNEK | (SEQ ID | PSSLSAS | ISLN | (SEQ ID | LT |
| 1111B12- | | GASVKLS | NO: 61) | FKS | NO: 43) | LGERVSL | (SEQ ID | NO: 46) | (SEQ ID |
| Ab2 | | CKASGYT | | (SEQ ID | | TCRASQD | NO: 65) | | NO: 67) |
| | | FTTYWMH | | NO: 62) | | IGISLNW | | | |
| | | WVKQRPG | | | | FQQEPDG | | | |
| | | QGLEWIG | | | | TIKRLIY | | | |
| | | NINPING | | | | ATSSLDS | | | |
| | | GSNYNEK | | | | GVPKRFS | | | |
| | | FKSKASL | | | | GNRSGSD | | | |
| | | TVDKSSS | | | | YSLTISS | | | |
| | | TAYMQLS | | | | LESEDFA | | | |
| | | SLTSEDS | | | | DYYCLQF | | | |
| | | AVYYCVI | | | | ASSPLTF | | | |
| | | AMDYWGQ | | | | GAGTKLE | | | |
| | | GTSVTVS | | | | LK | | | |
| | | S | | | | (SEQ ID | | | |
| | | (SEQ ID | | | | NO: 64) | | | |
| | | NO: 60) | | | | | | | |
| ACI- | 1112H8C12 | EVKLEES | DAWM | EIRNKAH | YSY | SIVMTQT | TASQSVS | SASNRFT | QQDYTSP |
| 7067- | | GGGLVQP | (SEQ ID | NYATYYA | | PKFLLMS | NYVA | (SEQ ID | YT |
| 1112H8- | | GGSMKLS | NO: 21) | ESVKG | | PGDRVTM | (SEQ ID | NO: 76) | (SEQ ID |
| Ab2 | | CAASGFT | | (SEQ ID | | TCTASQS | NO: 75) | | NO: 77) |
| | | FTDAWMN | | NO: 72) | | VSNYVAW | | | |
| | | WVRQSPE | | | | YQQKPGQ | | | |
| | | KGLEWIA | | | | SPKLLIY | | | |
| | | EIRNKAH | | | | SASNRFT | | | |
| | | NYATYYA | | | | GVPDRFT | | | |
| | | ESVKGRF | | | | GSGYGTD | | | |
| | | DISGDDS | | | | FTFTINT | | | |
| | | KSSVYLQ | | | | VQTEDMA | | | |
| | | MNNLRVE | | | | VYFCQQD | | | |
| | | DTGIYYC | | | | YTSPYTF | | | |
| | | TIYSYWG | | | | GGGTKLE | | | |
| | | PGTLVTV | | | | IK | | | |
| | | SA | | | | (SEQ ID | | | |
| | | (SEQ ID | | | | NO: 74) | | | |
| | | NO: 70) | | | | | | | |
| ACI- | 1108B11D3 | EVQLVES | TYAMH | RIRSKGS | GHGSSYF | QIVLTQS | SANSSVT | DTSNLAS | QQWKSHP |
| 7067- | | GGGLVQP | (SEQ ID | NYATNYA | SY | PAIMSAS | YMH | (SEQ ID | PT |
| 1108B11- | | KGSLKLS | NO: 31) | DSVKD | (SEQ ID | PGERITM | (SEQ ID | NO: 36) | (SEQ ID |
| Ab2 | | CAASGFT | | (SEQ ID | NO: 33) | TCSANSS | NO: 85) | | NO: 87) |
| | | FNTYAMH | | NO: 32) | | VTYMHWY | | | |
| | | WVRQAPG | | | | QQKSGTS | | | |
| | | KGLEWVA | | | | PKRWIYD | | | |
| | | RIRSKGS | | | | TSNLASG | | | |
| | | NYATNYA | | | | VPARFSG | | | |
| | | DSVKDRF | | | | SGSGTSY | | | |
| | | TISRDDS | | | | SLTISSM | | | |
| | | QSMLYLQ | | | | EAEDAAT | | | |
| | | MNNLKTE | | | | YYCQQWK | | | |
| | | DTAMYYC | | | | SHPPTFG | | | |
| | | VRGHGSS | | | | AGTKLEL | | | |
| | | YFSYWGQ | | | | K | | | |
| | | GTLVTVS | | | | (SEQ ID | | | |
| | | A | | | | NO: 84) | | | |
| | | (SEQ ID | | | | | | | |
| | | NO: 30) | | | | | | | |
| ACI- | 1113D10 | EVQLVES | TYALH | RIRSKSS | GGVSPFD | QIVLTQS | SASSSVS | DTSKLAS | QQWSNNP |
| 7067- | E3D5 | GGGLVQP | (SEQ ID | NYATYYA | Y | PAIMSAS | YMH | (SEQ ID | PT |
| 1113D10- | | KGSLKLS | NO: 91) | DSVKD | (SEQ ID | PGEKVTM | (SEQ ID | NO: 96) | (SEQ ID |
| Ab1 | | CAASGFT | | (SEQ ID | NO: 93) | TCSASSS | NO: 95) | | NO: 97) |
| | | FNTYALH | | NO: 92) | | VSYMHWY | | | |
| | | WVRQAPG | | | | QQKSGTS | | | |
| | | KGLEWVA | | | | PKRWIYD | | | |
| | | RIRSKSS | | | | TSKLASG | | | |
| | | NYATYYA | | | | VPARFSG | | | |
| | | DSVKDRF | | | | SGSGTSY | | | |
| | | TISRDDS | | | | SLTISSM | | | |
| | | QSMLYLQ | | | | EAEDSAT | | | |
| | | MNNLKTE | | | | YYCQQWS | | | |
| | | DTGMYYC | | | | NNPPTFG | | | |
| | | VRGGVSP | | | | GGTKLEI | | | |
| | | FDYWGQG | | | | K | | | |
| | | TTLTVSS | | | | (SEQ ID | | | |
| | | (SEQ ID | | | | NO: 94) | | | |
| | | NO: 90) | | | | | | | |
| ACI- | 1116F2A2 | DVQLQES | RGFYWN | YISDDGN | GDLL | DVVMTQT | KSSQSLL | LVSKLDS | WQGTHFP |
| 7067- | | GPGFVKP | (SEQ ID | SNYNPSL | (SEQ ID | ALTLSVT | DSDGETY | (SEQ ID | QT |
| 1116F2- | | SQSLSLT | NO: 101) | KN | NO: 103) | IGQPASI | LN | NO: 106) | (SEQ ID |
| Ab1 | | CSVTGYS | | (SEQ ID | | SCKSSQS | (SEQ ID | | NO: 107) |
| | | ITRGFYW | | NO: 102) | | LLDSDGE | NO: 105) | | |
| | | NWIRQFP | | | | TYLNWLL | | | |
| | | GNKLEWM | | | | QRPGQSP | | | |
| | | GYISDDG | | | | KRLIYLV | | | |
| | | NSNYNPS | | | | SKLDSGV | | | |
| | | LKNRISI | | | | PDRFTGS | | | |
| | | TRDTFKN | | | | GSGTDFA | | | |
| | | QVFLRLN | | | | LKISRVE | | | |
| | | SVTTEDT | | | | AEDLGIY | | | |
| | | ATYYCTR | | | | YCWQGTH | | | |
| | | GDLLWGQ | | | | FPQTFGG | | | |
| | | GTTLTVS | | | | GTKLEIK | | | |
| | | S | | | | (SEQ ID | | | |
| | | (SEQ ID | | | | NO: 104) | | | |
| | | NO: 100) | | | | | | | |
| ACI- | 1206E5D2 | QVQLQQS | DYVIS | EIYPGND | EGVSNGY | DVLMTQT | KSSQSLL | LVSKLDS | VQGTHFP |
| 7067- | | GPELVKP | (SEQ ID | STYYNEK | LYLSMDY | PLTLSVT | YSNGKTY | (SEQ ID | WT |
| 1206E5- | | GASVKMS | NO: 111) | FKG | (SEQ ID | IGQPASI | LN | NO: 106) | (SEQ ID |
| Ab1 | | CKASGYT | | (SEQ ID | NO: 113) | SCKSSQS | (SEQ ID | | NO: 117) |
| | | FTDYVIS | | NO: 112) | | LLYSNGK | NO: 115) | | |
| | | WVKQGTG | | | | TYLNWLL | | | |
| | | QGLEWIG | | | | QRPGQSP | | | |
| | | EIYPGND | | | | KRLIYLV | | | |
| | | STYYNEK | | | | SKLDSGV | | | |
| | | FKGKATL | | | | PDRFTGS | | | |
| | | TADKSSN | | | | GSGTDFT | | | |
| | | TAYMQLS | | | | LKISRVE | | | |
| | | SLTSEDS | | | | AEDLGVY | | | |
| | | AVYFCAR | | | | YCVQGTH | | | |
| | | EGVSNGY | | | | FPWTFGG | | | |
| | | LYLSMDY | | | | GTKLEIK | | | |
| | | WGQGTSV | | | | (SEQ ID | | | |
| | | TVSS | | | | NO: 114) | | | |
| | | (SEQ ID | | | | | | | |
| | | NO: 110) | | | | | | | |
| ACI- | 2501B11C7 | QVQLQQS | SFWM | RIYPGDG | KGDFYGS | QAVVTQE | RSSTGAV | GTNNRAP | ALWYSNH |
| 7079- | | GPELVKP | (SEQ ID | DAHYNGE | NYDY | SALTTSP | TTSNYAN | (SEQ ID | LV |
| 2501B11- | | GASVKIS | NO: 281) | FKG | (SEQ ID | GETVTLT | (SEQ ID | NO: 286) | (SEQ ID |
| Ab3 | | CKASGYA | | (SEQ ID | NO: 283) | CRSSTGA | NO: 285) | | NO: 287) |
| | | FSSFWMN | | NO: 282) | | VTTSNYA | | | |
| | | WMKQRPG | | | | NWVQEKP | | | |
| | | KGLEWIG | | | | DHLFTGL | | | |
| | | RIYPGDG | | | | IGGTNNR | | | |
| | | DAHYNGE | | | | APGVPAR | | | |
| | | FKGRATL | | | | FSGSLIG | | | |
| | | TADKSSS | | | | DKAALTI | | | |
| | | TAYMQLS | | | | TGAQTED | | | |
| | | SLTSEDS | | | | EAIYFCA | | | |
| | | AVYFCAR | | | | LWYSNHL | | | |
| | | KGDFYGS | | | | VFGGGTR | | | |
| | | NYDYWGQ | | | | LTVL | | | |
| | | GTTLTVS | | | | (SEQ ID | | | |
| | | S | | | | NO: 284) | | | |
| | | (SEQ ID | | | | | | | |
| | | NO: 280) | | | | | | | |
| ACI- | 2501D10C3 | EVQLVES | TYAMH | RIRSENS | GYNGSSL | QIVLTQS | SASSSVN | DTSKLAS | QQWRSNP |
| 7079- | | GGGLVQP | (SEQ ID | NFAKYYA | DY | PAIMSAF | YMH | (SEQ ID | PT |
| 2501D10- | | KGSLKVS | NO: 31) | DSVKD | (SEQ ID | PGERVTM | (SEQ ID | NO: 96) | (SEQ ID |
| Ab1 | | CAASGFT | | (SEQ ID | NO: 193) | TCSASSS | NO: 195) | | NO: 197) |
| | | FKTYAMH | | NO: 192) | | VNYMHWY | | | |
| | | WVRQAPG | | | | QQKSGTS | | | |
| | | KGLEWVA | | | | PKRWIYD | | | |
| | | RIRSENS | | | | TSKLASG | | | |
| | | NFAKYYA | | | | VPARFSG | | | |
| | | DSVKDRF | | | | GGSGTSY | | | |
| | | TISRDDS | | | | SLTISNM | | | |
| | | QSMLYLQ | | | | EAEDAAT | | | |
| | | MNNLKTE | | | | YYCQQWR | | | |
| | | DTAMYYC | | | | SNPPTFG | | | |
| | | VRGYNGS | | | | GGTKLEI | | | |
| | | SLDYWGQ | | | | K | | | |
| | | GTTLTVS | | | | (SEQ ID | | | |
| | | S | | | | NO: 194) | | | |
| | | (SEQ ID | | | | | | | |
| | | NO: 290) | | | | | | | |
| ACI- | 2501G2E5 | EVQLVES | TYAMN | RIRTKSN | QGLAYYA | DVLMTQT | RSSQTIV | KVSNRFS | FQGSQGP |
| 7079- | | GGGLVQP | (SEQ ID | NFATYYA | MDY | PLSLPVS | HSNGNTY | (SEQ ID | LT |
| 2501G2- | | KGSLKLS | NO: 141) | HSVKD | (SEQ ID | LGDQVSI | LE | NO: 16) | (SEQ ID |
| Ab2 | | CAASGFN | | (SEQ ID | NO: 143) | SCRSSQT | (SEQ ID | | NO: 17) |
| | | FNTYA | | NO: 142) | | IVHSNGN | NO: 145) | | |
| | | MNWVRQA | | | | TYLEWYL | | | |
| | | PGK | | | | QKPGQSP | | | |
| | | GLEWVAR | | | | KLLIYKV | | | |
| | | IRTKSNN | | | | SNRFSGV | | | |
| | | FATYYAH | | | | PDRFSGS | | | |
| | | SVKDRFT | | | | GSGTDFT | | | |
| | | ISRDDSE | | | | LKISRVE | | | |
| | | SMLYLQM | | | | AEDLGVY | | | |
| | | NNLKTED | | | | YCFQGSQ | | | |
| | | TAMYYCV | | | | GPLTFGA | | | |
| | | RQGLAYY | | | | GTKLELK | | | |
| | | AMDYWGQ | | | | (SEQ ID | | | |
| | | GTSVTVS | | | | NO: 144) | | | |
| | | S | | | | | | | |
| | | (SEQ ID | | | | | | | |
| | | NO: 140) | | | | | | | |
| ACI- | 2503C6H9 | DVQLQES | SGYYWN | YINYDGS | GDWD | DVVMTQT | KSSQSLL | LVSKLDS | WQGTHFP |
| 7079- | | GPGLVKP | (SEQ ID | NNFNPSL | (SEQ ID | PLTLSVT | DSDGETY | (SEQ ID | QT |
| 2503C6- | | SQSLSLT | NO: 151) | KN | NO: 153) | IGQPASI | LN | NO: 106) | (SEQ ID |
| Ab1 | | CSVTGYS | | (SEQ ID | | SCKSSQS | (SEQ ID | | NO: 107) |
| | | ITSGYYW | | NO: 152) | | LLDSDGE | NO: 105) | | |
| | | NWIRLFP | | | | TYLNWLL | | | |
| | | GNKLEWL | | | | QRPGQSP | | | |
| | | GYINYDG | | | | KRLICLV | | | |
| | | SNNFNPS | | | | SKLDSGV | | | |
| | | LKNRISI | | | | PDRFTGS | | | |
| | | TRDTSKN | | | | GSGTDFT | | | |
| | | QFFLKLN | | | | LKISRVE | | | |
| | | SVTSEDT | | | | AEDLGVY | | | |
| | | ATYFCLR | | | | YCWQGTH | | | |
| | | GDWDWGQ | | | | FPQTFGG | | | |
| | | GTLVTVS | | | | GTRLEIK | | | |
| | | A | | | | (SEQ ID | | | |
| | | (SEQ ID | | | | NO: 154) | | | |
| | | NO: 150) | | | | | | | |
| ACI- | 2504A6C8 | QVQLQQS | SYGIS | EIYPGSG | DYDAY | DVVMTQT | RSSQSLV | KVSNRFS | SQSTHVP |
| 7079- | | GVELARP | (SEQ ID | NTYYNEK | (SEQ ID | PLSLPVS | HSNGNTY | (SEQ ID | LT |
| 2504A6- | | GASVKLS | NO: 161) | FKG | NO: 163) | LGDQASI | LH | NO: 16) | (SEQ ID |
| Ab1 | | CKASGYT | | (SEQ ID | | SCRSSQS | (SEQ ID | | NO: 167) |
| | | FTSYGIS | | NO: 162) | | LVHSNGN | NO: 165) | | |
| | | WVKQRTG | | | | TYLHWYL | | | |
| | | QGLKWIG | | | | QKPGQSP | | | |
| | | EIYPGSG | | | | KLLIYKV | | | |
| | | NTYYNEK | | | | SNRFSGV | | | |
| | | FKGKATL | | | | PDRFSGS | | | |
| | | TADKSSS | | | | GSGTDFT | | | |
| | | TAYMELR | | | | LKISRVE | | | |
| | | SLTSEDS | | | | AEDLGVY | | | |
| | | AVYFCAT | | | | FCSQSTH | | | |
| | | DYDAYWG | | | | VPLTFGA | | | |
| | | QGTTLTV | | | | GTKLELK | | | |
| | | SS | | | | (SEQ ID | | | |
| | | (SEQ ID | | | | NO: 164) | | | |
| | | NO: 160) | | | | | | | |
| ACI- | 2506E2G4 | QVQLQQS | NSWMN | RIFPGDG | WGGTNDE | DIVLTQS | RASQSVS | YASNLES | QHSWDIP |
| 7079- | | GPELVRP | (SEQ ID | DTYYDGK | WFAH | PASLTVS | TSRNSYM | (SEQ ID | LT |
| 2506E2- | | GASVKIS | NO: 171) | FKG | (SEQ ID | LGQRATI | H | NO: 176) | (SEQ ID |
| Ab2 | | CKASGYA | | (SEQ ID | NO: 173) | SCRASQS | (SEQ ID | | NO: 177) |
| | | FSNSWMN | | NO: 172) | | VSTSRNS | NO: 175) | | |
| | | WVKQRPG | | | | YMHWYQQ | | | |
| | | KGLEWIG | | | | KPRQPPK | | | |
| | | RIFPGDG | | | | LLIKYAS | | | |
| | | DTYYDGK | | | | NLESGVP | | | |
| | | FKGKVKL | | | | ARFSGSG | | | |
| | | TTDKFSN | | | | SGADFTL | | | |
| | | TAYMQLR | | | | NIHPVEE | | | |
| | | SLTSEDS | | | | EDTATYY | | | |
| | | AVYFCAR | | | | CQHSWDI | | | |
| | | WGGTNDE | | | | PLTFGTG | | | |
| | | WFAHWGQ | | | | TKLELS | | | |
| | | GTLVTVS | | | | (SEQ ID | | | |
| | | V | | | | NO: 174) | | | |
| | | (SEQ ID | | | | | | | |
| | | NO: 170) | | | | | | | |
| ACI- | 2506F3E12 | QVQLQQP | TYWMQ | EIDPSDS | GMMDY | DVLMTQT | RSSQSIV | KVSNRFS | FKGSHVP |
| 7079- | | GAELVKP | (SEQ ID | YINYNQK | (SEQ ID | PLSLPVS | HSNGNTY | (SEQ ID | YT |
| 2506F3- | | GASVKLS | NO: 181) | FKG | NO: 183) | LGDQASI | LE | NO: 16) | (SEQ ID |
| Ab1 | | CKASGYT | | (SEQ ID | | SCRSSQS | (SEQ ID | | NO: 187) |
| | | FTTYWMQ | | NO: 182) | | IVHSNGN | NO: 15) | | |
| | | WVKQRPG | | | | TYLEWYL | | | |
| | | QGLEWIG | | | | QKPGQSP | | | |
| | | EIDPSDS | | | | KLLIYKV | | | |
| | | YINYNQK | | | | SNRFSGV | | | |
| | | FKGKATL | | | | PDRFSGS | | | |
| | | TVDTSSS | | | | GSGTDFT | | | |
| | | TAFMQLS | | | | LKISRVE | | | |
| | | SLTSEDS | | | | AEDLGVY | | | |
| | | AVYYCAR | | | | YCFKGSH | | | |
| | | GMMDYWG | | | | VPYTFGG | | | |
| | | QGTSVTV | | | | GTKLEIK | | | |
| | | SS | | | | (SEQ ID | | | |
| | | (SEQ ID | | | | NO: 184) | | | |
| | | NO: 180) | | | | | | | |
| ACI- | 2507B3G8 | EVQLVES | TYAMH | RIRSENS | GYNGSSL | QIVLTQS | SASSSVN | DTSKLAS | QQWRSNP |
| 7079- | | GGGLVQP | (SEQ ID | NFAKYYA | DY | PAIMSAF | YMH | (SEQ ID | PT |
| 2507B3- | | KGSLKVS | NO: 31) | DSVKD | (SEQ ID | PGERVTM | (SEQ ID | NO: 96) | (SEQ ID |
| Ab1 | | CAASGFT | | (SEQ ID | NO: 193) | TCSASSS | NO: 195) | | NO: 197) |
| | | FKTYAMH | | NO: 192) | | VNYMHWY | | | |
| | | WVRQAPG | | | | QQKSGTS | | | |
| | | KGLEWVA | | | | PKRWIYD | | | |
| | | RIRSENS | | | | TSKLASG | | | |
| | | NFAKYYA | | | | VPARFSG | | | |
| | | DSVKDRF | | | | GGSGTSY | | | |
| | | TISRDDS | | | | SLTISNM | | | |
| | | QSMLYLQ | | | | EAEDAAT | | | |
| | | MHTLKTE | | | | YYCQQWR | | | |
| | | DTAIYYC | | | | SNPPTFG | | | |
| | | VRGYNGS | | | | GGTKLEI | | | |
| | | SLDYWGQ | | | | K | | | |
| | | GTTLTVS | | | | (SEQ ID | | | |
| | | S | | | | NO: 194) | | | |
| | | (SEQ ID | | | | | | | |
| | | NO: 190) | | | | | | | |
| ACI- | 2511B3B12 | DVQLQES | SYYYWN | YISYDGS | GDWD | DVVMTQT | KSSQSLL | LVSKLES | WQGTHFP |
| 7079- | | GPGLVKP | (SEQ ID | NNYNPSL | (SEQ ID | PLTLSLT | DSDGETY | (SEQ ID | QT |
| 2511B3- | | SQSLSLT | NO: 201) | KN | NO: 153) | IGQPASI | LN | NO: 206) | (SEQ ID |
| Ab3 | | CSVTGFS | | (SEQ ID | | SCKSSQS | (SEQ ID | | NO: 107) |
| | | ITSYYYW | | NO: 202) | | LLDSDGE | NO: 105) | | |
| | | NWIRQFP | | | | TYLNWLL | | | |
| | | GNKLEWM | | | | QRPGQSP | | | |
| | | AYISYDG | | | | KRLIYLV | | | |
| | | SNNYNPS | | | | SKLESGV | | | |
| | | LKNRISI | | | | PDRFTGS | | | |
| | | TRDTSKN | | | | GSGTVFT | | | |
| | | QFFLKLN | | | | LKISRVE | | | |
| | | SVTTEDT | | | | AEDLGVY | | | |
| | | ATYYCTR | | | | YCWQGTH | | | |
| | | GDWDWGQ | | | | FPQTFGG | | | |
| | | GTLVTVS | | | | GTKLEIK | | | |
| | | A | | | | (SEQ ID | | | |
| | | (SEQ ID | | | | NO: 204) | | | |
| | | NO: 200) | | | | | | | |
| ACI- | 2601B6D2 | EIQLQQS | DDYIH | WIDPENG | RGFGY | DIVMTQS | KASQDVG | WASSRHT | QQYSSYP |
| 7079- | | GAELVRP | (SEQ ID | DTDYASK | (SEQ ID | HKFMSTS | NVVA | (SEQ ID | LT |
| 2601B6- | | GASVKLS | NO: 211) | FQG | NO: 213) | VGDRVSI | (SEQ ID | NO: 216) | (SEQ ID |
| Ab1 | | CTTSGFN | | (SEQ ID | | TCKASQD | NO: 215) | | NO: 217) |
| | | IKDDYIH | | NO: 212) | | VGNVVAW | | | |
| | | WVKQRPE | | | | YQQKPGQ | | | |
| | | QGLEWIG | | | | SPKLLIY | | | |
| | | WIDPENG | | | | WASSRHT | | | |
| | | DTDYASK | | | | GVPDRFT | | | |
| | | FQGKATI | | | | GSGSGTE | | | |
| | | TADTSSN | | | | FTLTISN | | | |
| | | TAYLHLS | | | | VQSEDLA | | | |
| | | SLTSEDA | | | | DYFCQQY | | | |
| | | AVYFCTT | | | | SSYPLTF | | | |
| | | RGFGYWG | | | | GAGTKLE | | | |
| | | QGTLVTV | | | | LK | | | |
| | | S | | | | (SEQ ID | | | |
| | | (SEQ ID | | | | NO: 214) | | | |
| | | NO: 210) | | | | | | | |
| ACI- | 2602G4H1 | EVQLVES | TYAMH | RIRSKGS | GGADSWF | QIVLTQS | TASSSVS | DTSKLAS | QQWNRNP |
| 7079- | | GGGLVQP | (SEQ ID | DYATYYA | AY | PAIMSAS | YMH | (SEQ ID | PT |
| 2602G4- | | KGSLKLS | NO: 31) | DSVKD | (SEQ ID | PGERITM | (SEQ ID | NO: 96) | (SEQ ID |
| Ab4 | | CAASGFT | | (SEQ ID | NO: 223) | TCTASSS | NO: 225) | | NO: 227) |
| | | FKTYAMH | | NO: 222) | | VSYMHWY | | | |
| | | WVRQAPG | | | | QQKSGTS | | | |
| | | KGLEWVA | | | | PKRWIYD | | | |
| | | RIRSKGS | | | | TSKLASG | | | |
| | | DYATYYA | | | | VPARFSG | | | |
| | | DSVKDRF | | | | SGSGASY | | | |
| | | TISRDDS | | | | TLTISSM | | | |
| | | QSMLYLQ | | | | EAEDAAT | | | |
| | | MNNLKTE | | | | YYCQQWN | | | |
| | | DTAMYFC | | | | RNPPTFG | | | |
| | | VRGGADS | | | | GGTQLAI | | | |
| | | WFAYWGQ | | | | K | | | |
| | | GTLVTVS | | | | (SEQ ID | | | |
| | | T | | | | NO: 224) | | | |
| | | (SEQ ID | | | | | | | |
| | | NO: 220) | | | | | | | |
| ACI- | 2603C1H6 | QVQLQQP | SYWMQ | EIDPSDS | YDGPSY | ENVLTQS | SAGSSVS | DTSKLPS | FQGSGYP |
| 7079- | | GADLVKP | (SEQ ID | YANYNQK | (SEQ ID | PAIMSAS | YMH | (SEQ ID | YT |
| 2603C1- | | GASVKLS | NO: 231) | FKG | NO: 233) | PGEKVTM | (SEQ ID | NO: 236) | (SEQ ID |
| Ab3 | | CKASGYT | | (SEQ ID | | TCSAGSS | NO: 235) | | NO: 237) |
| | | FTSYWMQ | | NO: 232) | | VSYMHWF | | | |
| | | WTKQRPG | | | | QQKSSTS | | | |
| | | QGLEWIG | | | | PKLWIYD | | | |
| | | EIDPSDS | | | | TSKLPSG | | | |
| | | YANYNQK | | | | VPGRFSG | | | |
| | | FKGKATL | | | | SGSGNSY | | | |
| | | TVDKYSS | | | | SLTISSM | | | |
| | | TAYMQLN | | | | EAEDVAT | | | |
| | | SLTSEDS | | | | YYCFQGS | | | |
| | | AVYYCAL | | | | GYPYTFG | | | |
| | | YDGPSYW | | | | GGTKLEI | | | |
| | | GQGTLVT | | | | K | | | |
| | | VS | | | | (SEQ ID | | | |
| | | (SEQ ID | | | | NO: 234) | | | |
| | | NO: 230) | | | | | | | |
| ACI- | 2603F3H3 | EVQLVES | TYAMH | RIRSKGS | GGGDSWF | QIVLTQS | TASSSVS | DTSKLAS | QQWNSNP |
| 7079- | | GGGLVQP | (SEQ ID | NYATYYA | AY | PAIMSAS | YMH | (SEQ ID | PT |
| 2603F3- | | KGSLKLS | NO: 31) | DSVKD | (SEQ ID | PGERVTM | (SEQ ID | NO: 96) | (SEQ ID |
| Ab1 | | CAASGFT | | (SEQ ID | NO: 243) | TCTASSS | NO: 225) | | NO: 247) |
| | | FNTYAMH | | NO: 242) | | VSYMHWY | | | |
| | | WVRQAPG | | | | QQKSGTS | | | |
| | | KGLEWVA | | | | PKRWIYD | | | |
| | | RIRSKGS | | | | TSKLASG | | | |
| | | NYATYYA | | | | VPARFSG | | | |
| | | DSVKDRF | | | | SGSGASY | | | |
| | | TISRDDS | | | | TLTISSM | | | |
| | | QSMLYLQ | | | | EAEDAAT | | | |
| | | MNNLKTE | | | | YYCQQWN | | | |
| | | DTAMYYC | | | | SNPPTFG | | | |
| | | VRGGGDS | | | | GGTQLAI | | | |
| | | WFAYWGQ | | | | K | | | |
| | | GTLVTVS | | | | (SEQ ID | | | |
| | | A | | | | NO: 244) | | | |
| | | (SEQ ID | | | | | | | |
| | | NO: 240) | | | | | | | |
| ACI- | 2605B3D1 | EVQLVES | TYAMH | RIRSKGG | GGNYSWF | QIVLTQS | SASSSVT | DTSQLAS | QQWTRNP |
| 7079- | | GGGLVQP | (SEQ ID | NYATYFA | AY | PAIMSAS | YMH | (SEQ ID | P |
| 2605B3- | | KGSLKLS | NO: 31) | DSVKD | (SEQ ID | PGEKVTM | (SEQ ID | NO: 256) | (SEQ ID |
| Ab2 | | CAASGFI | | (SEQ ID | NO: 253) | TCSASSS | NO: 255) | | NO: 257) |
| | | FKTYAMH | | NO: 252) | | VTYMHWY | | | |
| | | WVRQAPG | | | | QQKSGTS | | | |
| | | KGLEWVA | | | | PKRWIYD | | | |
| | | RIRSKGG | | | | TSQLASG | | | |
| | | NYATYFA | | | | VPARFSG | | | |
| | | DSVKDRF | | | | SGSGTSH | | | |
| | | TISRDDS | | | | SLTISSM | | | |
| | | QNMLYLQ | | | | ETEDAAT | | | |
| | | VNNLKIE | | | | YYCQQWT | | | |
| | | DTAMYFC | | | | RNPPTFG | | | |
| | | VRGGNYS | | | | GGTKLAI | | | |
| | | WFAYWGQ | | | | K | | | |
| | | GTLVTVSA | | | | (SEQ ID | | | |
| | | (SEQ ID | | | | NO: 254) | | | |
| | | NO: 250) | | | | | | | |
| ACI- | 2606A6D5 | QVQLQQS | SSWMN | RIFPGDG | WTGGYDW | DIVLTQS | RASQSVS | YASNLES | QHSWEIP |
| 7079- | | GPELVKP | (SEQ ID | DTNYDRK | FAY | PASLAVS | TSNYNYL | (SEQ ID | LT |
| 2606A6- | | GASVKIS | NO: 261) | FKD | (SEQ ID | LGQRATI | H | NO: 176) | (SEQ ID |
| Ab2 | | CKASGFA | | (SEQ ID | NO: 263) | SCRASQS | (SEQ ID | | NO: 267) |
| | | FSSSWMN | | NO: 262) | | VSTSNYN | NO: 265) | | |
| | | WVKQRPG | | | | YLHWYQQ | | | |
| | | KGLEWVG | | | | KPGQPPK | | | |
| | | RIFPGDG | | | | LLITYAS | | | |
| | | DTNYDRK | | | | NLESGVP | | | |
| | | FKDKATL | | | | ARFSGSG | | | |
| | | TADKSSS | | | | SGTDFTL | | | |
| | | TAYMQLS | | | | NIHPVEE | | | |
| | | SLTSEDS | | | | GDTATYY | | | |
| | | AVYFCAR | | | | CQHSWEI | | | |
| | | WTGGYDW | | | | PLTFGAG | | | |
| | | FAYWGQG | | | | TKLELK | | | |
| | | TLVTVSA | | | | (SEQ ID | | | |
| | | (SEQ ID | | | | NO: 264) | | | |
| | | NO: 260) | | | | | | | |
| ACI- | 2509E5E5 | EVQLQQS | DYNMH | YINPNNG | GGDHRFA | DIVLTQS | RASESVD | SASYKGS | QQNKEVP |
| 7079- | | GPELVKP | (SEQ ID | VPTYKQK | Y | PASLAVS | YYGFSFV | (SEQ ID | LT |
| 2509E5- | | GASLKMS | NO: 271) | FKG | (SEQ ID | LGQRATI | N | NO: 276) | (SEQ ID |
| Ab2 | | CKASGYS | | (SEQ ID | NO: 273) | SCRASES | (SEQ ID | | NO: 277) |
| | | FTDYNMH | | NO: 272) | | VDYYGFS | NO: 275) | | |
| | | WVKQSRG | | | | FVNWFQQ | | | |
| | | KSLEWIG | | | | KPGQPPK | | | |
| | | YINPNNG | | | | LLIYSAS | | | |
| | | VPTYKQK | | | | YKGSGVP | | | |
| | | FKGRATL | | | | VRFSGSG | | | |
| | | TVNQSSS | | | | SGTDFSL | | | |
| | | TAYMEIR | | | | SIHPMEA | | | |
| | | SLTSEDS | | | | DDTAMYF | | | |
| | | AVYYCTR | | | | CQQNKEV | | | |
| | | GGDHRFA | | | | PLTFGAG | | | |
| | | YWGQGTL | | | | TKLELK | | | |
| | | VTVSA | | | | (SEQ ID | | | |
| | | (SEQ ID | | | | NO: 274) | | | |
| | | NO: 270) | | | | | | | |
| ACI- | 4119E10D12 | QVQLQQS | DYEMN | AIDPETG | FLLIDFD | DVLMTQT | RSSQSIV | KVSNRFS | FQGSHVP |
| 7087- | | GAELVRP | (SEQ ID | GTAYNQK | Y | PLSLPVS | HSNGNTY | (SEQ ID | YT |
| 4119E10- | | GASVTLS | NO: 301) | FKG | (SEQ ID | LGDQASI | LE | NO: 16) | (SEQ ID |
| Ab2 | | CKASGYT | | (SEQ ID | NO: 303) | SCRSSQS | (SEQ ID | | NO: 307) |
| | | FSDYEMN | | NO: 302) | | IVHSNGN | NO: 15) | | |
| | | WVKQTPV | | | | TYLEWYL | | | |
| | | HGLEWIG | | | | KKAGQSP | | | |
| | | AIDPETG | | | | KVLIYKV | | | |
| | | GTAYNQK | | | | SNRFSGV | | | |
| | | FKGKAIL | | | | PDRFSGS | | | |
| | | TSDKSSS | | | | GSGTDFT | | | |
| | | TAYMELR | | | | LKISRVE | | | |
| | | SLTSEDS | | | | AEDLGVY | | | |
| | | AVYYCTR | | | | YCFQGSH | | | |
| | | FLLIDFD | | | | VPYTFGG | | | |
| | | YWGQGTT | | | | GTELEIK | | | |
| | | LTVSS | | | | (SEQ ID | | | |
| | | (SEQ ID | | | | NO: | | | |
| | | NO: 300) | | | | 304) | | | |
| ACI- | 4125E6D5 | QVQLQQP | SYWMH | NINPSNG | GLHY | DIQMTQS | RASQDIG | ATSSLDS | LQFASSP |
| 7087- | | GTELVKP | (SEQ ID | GTNYNEK | (SEQ ID | PSSLSAS | NYLN | (SEQ ID | LT |
| 4125E6- | | GASVRLS | NO: 311) | FKN | NO: 313) | LGERVTL | (SEQ ID | NO: 46) | (SEQ ID |
| Ab1 | | CKASGYA | | (SEQ ID | | TCRASQD | NO: 315) | | NO: 67) |
| | | FTSYWMH | | NO: 312) | | IGNYLNW | | | |
| | | WVKQRPG | | | | LQQEPDG | | | |
| | | QGLEWIG | | | | TIKRLIY | | | |
| | | NINPSNG | | | | ATSSLDS | | | |
| | | GTNYNEK | | | | GVPKRFS | | | |
| | | FKNKATL | | | | GSRSGSD | | | |
| | | TVDKSSS | | | | YSLTISS | | | |
| | | TAYMQLS | | | | LESEDFV | | | |
| | | GLTSEDS | | | | DYYCLQF | | | |
| | | AVYYCAT | | | | ASSPLTF | | | |
| | | GLHYWGQ | | | | GPGTKLE | | | |
| | | GTTLTVS | | | | LK | | | |
| | | S | | | | (SEQ ID | | | |
| | | (SEQ ID | | | | NO: 314) | | | |
| | | NO: 310) | | | | | | | |
| ACI- | 4301D5B10 | QVQLQQS | SYYIH | WIYPGSD | DYDVGFG | DIVLTQS | RASESVD | AASNQGS | QQSQEVP |
| 7088- | | GPELVRP | (SEQ ID | NTKHNDK | Y | PASLAVS | NYGISFM | (SEQ ID | LT |
| 4301D5- | | GASVKIS | NO: 321) | FKG | (SEQ ID | LGQRATI | N | NO: 326) | (SEQ ID |
| Ab2 | | CKASGYR | | (SEQ ID | NO: 323) | SCRASES | (SEQ ID | | NO: 327) |
| | | FTSYYIH | | NO: 322) | | VDNYGIS | NO: 325) | | |
| | | WVKQRPG | | | | FMNWFQQ | | | |
| | | QGLEWIG | | | | KPGQPPK | | | |
| | | WIYPGSD | | | | LLIYAAS | | | |
| | | NTKHNDK | | | | NQGSGVP | | | |
| | | FKGKATL | | | | ARFSGIG | | | |
| | | TADTSSS | | | | SGTDFSL | | | |
| | | TAYMQLS | | | | NIHPMEE | | | |
| | | SLTSEDS | | | | DDTAMYF | | | |
| | | AVYFCAR | | | | CQQSQEV | | | |
| | | DYDVGFG | | | | PLTFGAG | | | |
| | | YWGQGTL | | | | TKLELK | | | |
| | | VTVSS | | | | (SEQ ID | | | |
| | | (SEQ ID | | | | NO: 324) | | | |
| | | NO: 320) | | | | | | | |
| ACI- | 4301E12B9 | DVQLQES | SGYYWN | YISDDGS | GDSR | DVVLTQT | RSSQNLL | LVSELDS | WQGTHFP |
| 7088- | | GPGLVKP | (SEQ ID | KNYNPSL | (SEQ ID | PLTLSVT | DSDGETY | (SEQ ID | QT |
| 4301E12- | | SQSLSLT | NO: 151) | KN | NO: 333) | IGQPASI | LN | NO: 336) | (SEQ ID |
| Ab2 | | CSVTGYS | | (SEQ ID | | SCRSSQN | (SEQ ID | | NO: 107) |
| | | ITSGYYW | | NO: 332) | | LLDSDGE | NO: 335) | | |
| | | NWIRQFP | | | | TYLNWLL | | | |
| | | GNKLEWM | | | | QRPGQSP | | | |
| | | GYISDDG | | | | KRLIYLV | | | |
| | | SKNYNPS | | | | SELDSGV | | | |
| | | LKNRISI | | | | PDRFTGS | | | |
| | | TRDTSKN | | | | GSGTDFT | | | |
| | | QLFMKLN | | | | LKISRVE | | | |
| | | SVTTEDT | | | | AEDLGVY | | | |
| | | ATYYCAR | | | | YCWQGTH | | | |
| | | GDSRLGQ | | | | FPQTFGG | | | |
| | | GTLVTVS | | | | GTKLEII | | | |
| | | A | | | | (SEQ ID | | | |
| | | (SEQ ID | | | | NO: 334) | | | |
| | | NO: 330) | | | | | | | |
| ACI- | 4301H3A5 | EVQLQQS | DYFMN | DINPNNG | GRNYAMD | DIVMSQS | KSSQSLL | SASTRES | KQSYDLW |
| 7088- | | GPELVKP | (SEQ ID | GTTYNQK | Y | PSSLAVS | NSRTRKN | (SEQ ID | T |
| 4301H3- | | GASVKIS | NO: 341) | FKG | (SEQ ID | AGEKVTM | YLA | NO: 346) | (SEQ ID |
| Ab2 | | CKASGYT | | (SEQ ID | NO: 343) | SCKSSQS | (SEQ ID | | NO: 347) |
| | | FADYFMN | | NO: 342) | | LLNSRTR | NO: 345) | | |
| | | WVKQSHG | | | | KNYLAWY | | | |
| | | KSLEWIG | | | | QQKPGQS | | | |
| | | DINPNNG | | | | PKLLIYS | | | |
| | | GTTYNQK | | | | ASTRESG | | | |
| | | FKGK | | | | VPDRFTG | | | |
| | | ATLTVDK | | | | SGFGTDF | | | |
| | | SSNTAYM | | | | TLTISSV | | | |
| | | ELRSLTS | | | | QAEDLAV | | | |
| | | EDSAVYY | | | | YYCKQSY | | | |
| | | CARGRNY | | | | DLWTFGG | | | |
| | | AMDYWGQ | | | | GTKLEIK | | | |
| | | GTSVTVS | | | | (SEQ ID | | | |
| | | S | | | | NO: 344) | | | |
| | | (SEQ ID | | | | | | | |
| | | NO: 340) | | | | | | | |
| ACI- | 4303A1E7 | EVQLQQS | DDYMH | WIDPENG | WGTAQAL | DIVMTQS | KASQNVG | SASNRYT | QQYRSYP |
| 7088- | | GAELVRP | (SEQ ID | DSEYASK | FPY | QKFMSTS | TSVG | (SEQ ID | LT |
| 4303A1- | | GASVKLS | NO: 351) | FQG | (SEQ ID | VGDRVSI | (SEQ ID | NO: 356) | (SEQ ID |
| Ab1 | | CTASGFN | | (SEQ ID | NO: 353) | TCKASQN | NO: 355) | | NO: 357) |
| | | IKDDYMH | | NO: 352) | | VGTSVGW | | | |
| | | WVKQRPE | | | | YQQKAGQ | | | |
| | | QGLEWIG | | | | SPKLLIH | | | |
| | | WIDPENG | | | | SASNRYT | | | |
| | | DSEYASK | | | | GVPDRFT | | | |
| | | FQGKATM | | | | GSGSGTD | | | |
| | | TADTSSN | | | | FTLTINN | | | |
| | | TAYLQLS | | | | MQSEDLA | | | |
| | | SLTSEDT | | | | DYFCQQY | | | |
| | | AVYYCKT | | | | RSYPLTF | | | |
| | | WGTAQAL | | | | GAGTKLE | | | |
| | | FPYWGQG | | | | LK | | | |
| | | TLVTVSA | | | | (SEQ ID | | | |
| | | (SEQ ID | | | | NO: 354) | | | |
| | | NO: 350) | | | | | | | |
| ACI- | 4303A3E4 | QVQLQQS | DYEMH | VIDPETG | GAALRLA | DVLMTQT | RSSQSIV | KVSNRFS | FQGSHVP |
| 7088- | | GAELVRP | (SEQ ID | GAVQNQK | Y | PLSLPVS | HSNGNSY | (SEQ ID | FT |
| 4303A3- | | GASVTLS | NO: 361) | FKG | (SEQ ID | LGDQASI | LE | NO: 16) | (SEQ ID |
| Ab1 | | CKASGYT | | (SEQ ID | NO: 363) | SCRSSQS | (SEQ ID | | NO: 367) |
| | | FTDYEMH | | NO: 362) | | IVHSNGN | NO: 365) | | |
| | | WVKQTPV | | | | SYLEWYL | | | |
| | | HGLEWIG | | | | QKPGQSP | | | |
| | | VIDPETG | | | | KLLIYKV | | | |
| | | GAVQNQK | | | | SNRFSGV | | | |
| | | FKGKAIL | | | | PDRFSGS | | | |
| | | TADNSSS | | | | GSGTDFT | | | |
| | | TAYMDLR | | | | LKINRVE | | | |
| | | SLTSEDS | | | | AEDLGVY | | | |
| | | AVYNCAM | | | | YCFQGSH | | | |
| | | GAALRLA | | | | VPFTFGS | | | |
| | | YWGQGTL | | | | GTKLEIK | | | |
| | | VTVSS | | | | (SEQ ID | | | |
| | | (SEQ ID | | | | NO: 364) | | | |
| | | NO: 360) | | | | | | | |
| ACI- | 4303B6C11 | EVQLQQS | DYYMN | DINPNNG | SGYSGSR | DIVMSQS | KSSQSLL | WASTRES | KQSYDLW |
| 7088- | | GPELVKP | (SEQ ID | GTTYNQK | LYYAMDY | PSSLAVS | NSRTRKN | (SEQ ID | T |
| 4303B6- | | GASVKIS | NO: 371) | FKD | (SEQ ID | AREKVTM | YLA | NO: 376) | (SEQ ID |
| Ab2 | | CKASGYT | | (SEQ ID | NO: 373) | SCKSSQS | (SEQ ID | | NO: 347) |
| | | FTDYYMN | | NO: 372) | | LLNSRTR | NO: 345) | | |
| | | WVKQSHG | | | | KNYLAWY | | | |
| | | KSLEWIG | | | | QQKPGQS | | | |
| | | DINPNNG | | | | PKLLIFW | | | |
| | | GTTYNQK | | | | ASTRESG | | | |
| | | FKDKATL | | | | VPDRFTG | | | |
| | | TVDRSSS | | | | SGSGTDF | | | |
| | | TAYMELR | | | | TLTISSV | | | |
| | | SLTSGDS | | | | QAEDLAV | | | |
| | | AVYYCAR | | | | YYCKQSY | | | |
| | | SGYSGSR | | | | DLWTFGG | | | |
| | | LYYAMDY | | | | GTKLEIK | | | |
| | | WSQGSSV | | | | (SEQ ID | | | |
| | | TVSS | | | | NO: 374) | | | |
| | | (SEQ ID | | | | | | | |
| | | NO: 370) | | | | | | | |
| ACI- | 4303H6D7 | EVQLQQS | DDYMH | WIDPENG | AGSGVQL | DILMTQS | KASQNVG | SASSRYS | QQYNRYP |
| 7088- | | GAELVRP | (SEQ ID | DTEYASK | FDY | QKFMSTS | TNVA | (SEQ ID | LT |
| 4303H6- | | GASVKLS | NO: 351) | FQG | (SEQ ID | VGDRVSV | (SEQ ID | NO: 386) | (SEQ ID |
| Ab1 | | CTASGFN | | (SEQ ID | NO: 383) | TCKASQN | NO: 385) | | NO: 387) |
| | | IQDDYMH | | NO: 382) | | VGTNVAW | | | |
| | | WVKQRPE | | | | YQQKPGQ | | | |
| | | QGLEWIG | | | | SPKPLIS | | | |
| | | WIDPENG | | | | SASSRYS | | | |
| | | DTEYASK | | | | GVPDRFT | | | |
| | | FQGKATL | | | | GSGSGTD | | | |
| | | TADTSSN | | | | FTLTISN | | | |
| | | TAYLQLS | | | | VQSEDLA | | | |
| | | RLTSEDT | | | | DYFCQQY | | | |
| | | AVYYCTT | | | | NRYPLTF | | | |
| | | AGSGVQL | | | | GAGTKLE | | | |
| | | FDYWGQG | | | | LK | | | |
| | | TTLTVSA | | | | (SEQ ID | | | |
| | | (SEQ ID | | | | NO: 384) | | | |
| | | NO: 380) | | | | | | | |
| ACI- | 4305H7A4 | EVQLQQS | DDYMH | WIDPENG | WGTTQAL | DIVMTQS | KASQNVG | SASNRYT | QQYRSYP |
| 7088- | | GAELVRP | (SEQ ID | DTEYASK | FPY | QKFMSTS | TAVG | (SEQ ID | LT |
| 4305H7- | | GASVKLS | NO: 351) | FQG | (SEQ ID | VGDRVSI | (SEQ ID | NO: 356) | (SEQ ID |
| Ab1 | | CTASGFN | | (SEQ ID | NO: 393) | TCKASQN | NO: 395) | | NO: 357) |
| | | IKDDYMH | | NO: 382) | | VGTAVGW | | | |
| | | WVKQRPE | | | | YQQKAGQ | | | |
| | | QGLEWIG | | | | SPKLLIH | | | |
| | | WIDPENG | | | | SASNRYT | | | |
| | | DTEYASK | | | | GVPDRFT | | | |
| | | FQGKATM | | | | GSGSGTD | | | |
| | | IADTSSN | | | | FTLTINN | | | |
| | | TAYLQLS | | | | MQSEDLA | | | |
| | | SLTSEDT | | | | DYFCQQY | | | |
| | | AVYYCKT | | | | RSYPLTF | | | |
| | | WGTTQAL | | | | GAGTKLE | | | |
| | | FPYWGQG | | | | LK | | | |
| | | TLVTVSS | | | | (SEQ ID | | | |
| | | (SEQ ID | | | | NO: 394) | | | |
| | | NO: 390) | | | | | | | |
| ACI- | 4317A4D2 | EVQLQQS | DDYMH | WIDPENG | WGTTQAL | DIVMTQS | KASQNVG | SASNRYT | QQYRSYP |
| 7088- | | GAELVRP | (SEQ ID | DTEYASK | FPY | QKFMYTS | NAVG | (SEQ ID | LT |
| 4317A4- | | GASVKLS | NO: 351) | FQG | (SEQ ID | VGDRVSI | (SEQ ID | NO: 356) | (SEQ ID |
| Ab1 | | CTASGFN | | (SEQ ID | NO: 393) | TCKASQN | NO: 405) | | NO: 357) |
| | | IKDDYMH | | NO: 382) | | VGNAVGW | | | |
| | | WVKQRPE | | | | YQQKAGQ | | | |
| | | QGLEWIG | | | | SPKLLIH | | | |
| | | WIDPENG | | | | SASNRYT | | | |
| | | DTEYASK | | | | GVPDRFT | | | |
| | | FQGKATM | | | | GTGSGTD | | | |
| | | TADTSSN | | | | FTLTINN | | | |
| | | TAYLQLS | | | | MQSEDLA | | | |
| | | SLTSEDT | | | | DYFCQQY | | | |
| | | AVYYCKT | | | | RSYPLTF | | | |
| | | WGTTQAL | | | | GAGTKLE | | | |
| | | FPYWGQG | | | | LK | | | |
| | | TLVTVSA | | | | (SEQ ID | | | |
| | | (SEQ ID | | | | NO: 404) | | | |
| | | NO: 400) | | | | | | | |
| ACI- | 4409F1A8 | DVQLQES | RGYYWN | YISYDGS | GDSN | DVVMTQT | KSSQSLL | LVSKLDS | WQGTHFP |
| 7089- | | GPGLVKP | (SEQ ID | NNYNPSL | (SEQ ID | PLTLSVT | DSDGETY | (SEQ ID | QT |
| 4409F1- | | SQSLSLT | NO: 411) | RN | NO: 413) | IGQPASI | LN | NO: 106) | (SEQ ID |
| Ab1 | | CSVTGYS | | (SEQ ID | | SCKSSQS | (SEQ ID | | NO: 107) |
| | | ITRGYYW | | NO: 412) | | LLDSDGE | NO: 105) | | |
| | | NWIRQFP | | | | TYLNWLL | | | |
| | | GNKLEWM | | | | QRPGQSP | | | |
| | | GYISYDG | | | | KRLIYLV | | | |
| | | SNNYNPS | | | | SKLDSGV | | | |
| | | LRNRISI | | | | PDRFTGS | | | |
| | | TRDTSKN | | | | GSGTDFT | | | |
| | | QFFLKLK | | | | LKISRVE | | | |
| | | SVTTEDT | | | | AEDLGVY | | | |
| | | ATYFCAR | | | | YCWQGTH | | | |
| | | GDSNWGQ | | | | FPQTFGG | | | |
| | | GTTLTVS | | | | GTKLEIK | | | |
| | | A | | | | (SEQ ID | | | |
| | | (SEQ ID | | | | NO: 414) | | | |
| | | NO: 410) | | | | | | | |
| ACI- | 4415G5A11 | QVQLQQS | SSGIS | DIYPRSG | GNY | DIVITQD | RSSKSLL | LMSTRAS | QQLLEYP |
| 7089- | | GAELARP | (SEQ ID | NTYYNEK | | DLSNPVT | YKDGKTY | (SEQ ID | LT |
| 4415G5- | | GASVKVS | NO: 421) | FKD | | SGESVSI | LN | NO: 426) | (SEQ ID |
| Ab1 | | CKASGYT | | (SEQ ID | | SCRSSKS | (SEQ ID | | NO: 427) |
| | | FTSSGIS | | NO: 422) | | LLYKDGK | NO: 425) | | |
| | | WLKHRTG | | | | TYLNWFL | | | |
| | | QGLEWIG | | | | QRPGQSP | | | |
| | | DIYPRSG | | | | QLLIYLM | | | |
| | | NTYYNEK | | | | STRASGV | | | |
| | | FKDKATL | | | | SDRFSGS | | | |
| | | TADKSSS | | | | GSGTDFT | | | |
| | | TAYMELR | | | | LEISRVK | | | |
| | | SLTSEDS | | | | AEDVGVY | | | |
| | | AVYFCAS | | | | YCQQLLE | | | |
| | | GNYWGQG | | | | YPLTFGA | | | |
| | | TTLTVSA | | | | GTKLELK | | | |
| | | (SEQ ID | | | | (SEQ ID | | | |
| | | NO: 420) | | | | NO: 424) | | | |
| ACI- | 4417G6B12 | QVQLQQS | GYEMH | AIDPETG | GWDYFDY | DVVMTQT | RSSQSLL | RVSNRFS | SQSTHVP |
| 7089- | | GAELVRP | (SEQ ID | GTAYIQK | (SEQ ID | PLSLPVS | HSNGFTY | (SEQ ID | YT |
| 4417G6- | | GASVTLS | NO: 431) | FKG | NO: 433) | LGDQASI | LH | NO: 436) | (SEQ ID |
| Ab1 | | CKASGYT | | (SEQ ID | | SCRSSQS | (SEQ ID | | NO: 437) |
| | | FTGYEMH | | NO: 432) | | LLHSNGF | NO: 435) | | |
| | | KQTPVHG | | | | TYLHWYL | | | |
| | | LEWIGAI | | | | QKPGQSP | | | |
| | | DPETGGT | | | | KLLIYRV | | | |
| | | AYIQKFK | | | | SNRFSGV | | | |
| | | GKATLTA | | | | PDRFSGS | | | |
| | | DKSSSTA | | | | GSGTDFT | | | |
| | | YMELRSL | | | | LKISRVE | | | |
| | | TSEDSAV | | | | AEDLGVY | | | |
| | | YYCTRGW | | | | FCSQSTH | | | |
| | | DYFDYWG | | | | VPYTFGG | | | |
| | | QGTTLTV | | | | GTKLEIK | | | |
| | | SA | | | | (SEQ ID | | | |
| | | (SEQ ID | | | | NO: 434) | | | |
| | | NO: 430) | | | | | | | |
| ACI- | 4418C5G1 | DGQLQES | SGYYWN | YINYDGS | GDVY | DVVMTQT | KSSQSLL | LVSKLDS | WQGTHFP |
| 7089- | | GPGLVKP | (SEQ ID | NNYNPSL | (SEQ ID | PLTLSVT | DSDGETY | (SEQ ID | QT |
| 4418C5- | | SQSLSLT | NO: 151) | KN | NO: 443) | IGQPASI | LN | NO: 106) | (SEQ ID |
| Ab1 | | CSVTGYS | | (SEQ ID | | SCKSSQS | (SEQ ID | | NO: 107) |
| | | ITSGYYW | | NO: 442) | | LLDSDGE | NO: 105) | | |
| | | NWIRQFP | | | | TYLNWLL | | | |
| | | GNKLEWM | | | | QRPGQSP | | | |
| | | GYINYDG | | | | KRLIYLV | | | |
| | | SNNYNPS | | | | SKLDSGV | | | |
| | | LKNRISI | | | | PDRFTGS | | | |
| | | TRDTSKN | | | | GSGTDFT | | | |
| | | QFFLKFN | | | | LKISRVE | | | |
| | | FVTTEDT | | | | AEDLGVY | | | |
| | | ATYYCVR | | | | YCWQGTH | | | |
| | | GDVYWGQ | | | | FPQTFGG | | | |
| | | GTTLTVS | | | | GTKLEIK | | | |
| | | S | | | | (SEQ ID | | | |
| | | (SEQ ID | | | | NO: 414) | | | |
| | | NO: 440) | | | | | | | |
| ACI- | 4418F6G7 | QVQLQQS | SSGIS | DIYPRSG | GNY | DIVITQD | RSSKSLL | LMSTRAS | QQLLEYP |
| 7089- | | GAELARP | (SEQ ID | NTYYNEK | | DLSNPVT | YKDGKTY | (SEQ ID | LT |
| 4418F6- | | GASVKVS | NO: 421) | FKD | | SGESVSI | LN | NO: 426) | (SEQ ID |
| Ab1 | | CKASGYT | | (SEQ ID | | SCRSSKS | (SEQ ID | | NO: 427) |
| | | FTSSGIS | | NO: 422) | | LLYKDGK | NO: 425) | | |
| | | WLKHRTG | | | | TYLNWFL | | | |
| | | QGLEWIG | | | | QRPGQSP | | | |
| | | DIYPRSG | | | | QLLIYLM | | | |
| | | NTYYNEK | | | | STRASGV | | | |
| | | FKDKATL | | | | SDRFSGS | | | |
| | | TADKSSS | | | | GSGTDFT | | | |
| | | TAYMELR | | | | LEISRVK | | | |
| | | SLTSEDS | | | | AEDVGVY | | | |
| | | AVYFCSS | | | | YCQQLLE | | | |
| | | GNYWGQG | | | | YPLTFGA | | | |
| | | TTLTVSS | | | | GTKLELK | | | |
| | | (SEQ ID | | | | (SEQ ID | | | |
| | | NO: 450) | | | | NO: 424) | | | |
| ACI- | 917. | QVHLKQS | DYYIN | RIYPGSG | GYYGA | DVVMTQT | RSSQSLV | KVSNRFS | SQSTHVP |
| 8033- | 5A12A11C9 | GADLVRP | (SEQ ID | NTYYNEK | (SEQ ID | PLSLPVS | HSNGKTH | (SEQ ID | WT |
| 5A12- | | GASVKLS | NO: 461) | FKG | NO: 463) | LGDQASI | LH | NO: 16) | (SEQ ID |
| Ab1 | | CKASGYT | | (SEQ ID | | SCRSSQS | (SEQ ID | | NO: 467) |
| | | FTDYYIN | | NO: 462) | | LVHSNGK | NO: 465) | | |
| | | WVKQRPG | | | | THLHWYL | | | |
| | | QGLEWIA | | | | QKPGQSP | | | |
| | | RIYPGSG | | | | KLLIYKV | | | |
| | | NTYYNEK | | | | SNRFSGV | | | |
| | | FKGRATL | | | | PDRFSGS | | | |
| | | SAEKSST | | | | GSGTDFT | | | |
| | | TAYMQLS | | | | LKISRVE | | | |
| | | SLTSEDS | | | | AEDLGVY | | | |
| | | AVYFCVV | | | | FCSQSTH | | | |
| | | GYYGAWG | | | | VPWTFGG | | | |
| | | QGTTLTV | | | | GTKLEIK | | | |
| | | SS | | | | (SEQIDNO | | | |
| | | (SEQ ID | | | | .464) | | | |
| | | NO: 460) | | | | | | | |
| ACI- | 917. | EVQLVES | TYAMN | RIRSKSN | SFDY | DIKMTQS | KASQDIN | RAKRLVD | LQYDEFP |
| 8033- | 25A3E9F6 | GGGLVQP | (SEQ ID | NFATYYA | (SEQ ID | PSSMYAS | SYLS | (SEQ ID | FT |
| 25A3- | | KGSLKLS | NO: 141) | DSVKD | NO: 473) | LGERVTI | (SEQ ID | NO: 476) | (SEQ ID |
| Ab1 | | CAASGFS | | (SEQ ID | | TCKASQD | NO: 475) | | NO: 477) |
| | | FNTYAMN | | NO: 472) | | INSYLSW | | | |
| | | WVRQAPG | | | | FQQKPGK | | | |
| | | KGLEWVA | | | | SPKTLIY | | | |
| | | RIRSKSN | | | | RAKRLVD | | | |
| | | NFATYYA | | | | GVPSRFS | | | |
| | | DSVKDRF | | | | GSGSGQD | | | |
| | | TISRDES | | | | YSLTISS | | | |
| | | ESMLYLQ | | | | LEYEDMG | | | |
| | | MNNLKTE | | | | IYYCLQY | | | |
| | | DTAMYYC | | | | DEFPFTF | | | |
| | | VRSFDYW | | | | GSGTKLE | | | |
| | | GQGTTLT | | | | IK | | | |
| | | VSS | | | | (SEQ ID | | | |
| | | (SEQ ID | | | | NO: 474) | | | |
| | | NO: 470) | | | | | | | |
| ACI- | 917. | DVQLQES | TGNYRWS | YIYYSGT | IYYGNAM | DVVMTQT | RSSQSLV | KVSNRFS | SQSTHVP |
| 8033- | 1G10A10F6 | GPGLVKP | (SEQ ID | ITYNPSL | DY | PLSLPVS | HSNGNTY | (SEQ ID | HT |
| 1G10- | | SQTVFLT | NO: 481) | TS | (SEQ ID | LGDQASI | LH | NO: 16) | (SEQ ID |
| Ab1 | | CTVTGIS | | (SEQ ID | NO: 483) | SCRSSQS | (SEQ ID | | NO: 487) |
| | | ITTGNYR | | NO: 482) | | LVHSNGN | NO: 165) | | |
| | | WSWIRQF | | | | TYLHWYL | | | |
| | | PGNKLEW | | | | QKPGQSP | | | |
| | | IGYIYYS | | | | KLLIYKV | | | |
| | | GTITYNP | | | | SNRFSGV | | | |
| | | SLTSRTT | | | | PDRFSGS | | | |
| | | ITRDTPK | | | | GSGTDFT | | | |
| | | NQFFLEM | | | | LKISRVE | | | |
| | | NSLTAED | | | | AEDLGVY | | | |
| | | TATYYCA | | | | FCSQSTH | | | |
| | | RIYYGNA | | | | VPHTFGG | | | |
| | | MDYWGQG | | | | GTKLEIK | | | |
| | | TSVTVSS | | | | (SEQ ID | | | |
| | | (SEQ ID | | | | NO: 484) | | | |
| | | NO: 480) | | | | | | | |
| ACI- | 917. | EVQLVES | TYAMN | RIRSKSN | ESAY | DVVMTQT | RSSQSLV | KVSNRLS | SQSTHVP |
| 8033- | 19A2E9E5 | GGGLVQP | (SEQ ID | NYATYYV | (SEQ ID | PLSLPVS | HSNGNTY | (SEQ ID | FT |
| 19A2- | | KGSLKLS | NO: 141) | DSVKD | NO: 493) | LGDQASI | LY | NO: 496) | (SEQ ID |
| Ab1 | | CAASGFS | | (SEQ ID | | SCRSSQS | (SEQ ID | | NO: 497) |
| | | FNTYAMN | | NO: 492) | | LVHSNGN | NO: 495) | | |
| | | WVRQAPG | | | | TYLYWYL | | | |
| | | KGLEWVA | | | | QKPGQSP | | | |
| | | RIRSKSN | | | | KLLIYKV | | | |
| | | NYATYYV | | | | SNRLSGV | | | |
| | | DSVKDRF | | | | PDRFSGS | | | |
| | | TISRDDS | | | | GSGTDFT | | | |
| | | ESMLYLQ | | | | LKISRVE | | | |
| | | MNNLKTE | | | | AEDLGVY | | | |
| | | DTALYYC | | | | FCSQSTH | | | |
| | | VSESAYW | | | | VPFTFGS | | | |
| | | GQGTLVT | | | | GTKLEIK | | | |
| | | VSA | | | | (SEQ ID | | | |
| | | (SEQ ID | | | | NO: 494) | | | |
| | | NO: 490) | | | | | | | |
| ACI- | 917. | DVQLQES | SGYYWN | YISNDGS | GDQH | DVVLTQT | KSSQSLL | LVSELDS | WQGTHFP |
| 8033- | 8C10C6G3 | GPGLVKP | (SEQ ID | SKTNPSL | (SEQ ID | PLTLSVT | DSDGETY | (SEQ ID | QT |
| 8C10- | | SQSLSLT | NO: 151) | TN | NO: 503) | IGQPASI | LN | NO: 336) | (SEQ ID |
| Ab1 | | CSVTGQS | | (SEQ ID | | SCKSSQS | (SEQ ID | | NO: 107) |
| | | ITSGYYW | | NO: 502) | | LLDSDGE | NO: 105) | | |
| | | NWIRQFP | | | | TYLNWLL | | | |
| | | GNKLEWM | | | | QRPGQSP | | | |
| | | GYISNDG | | | | KRLIYLV | | | |
| | | SSKTNPS | | | | SELDSGV | | | |
| | | LTNRISV | | | | SDRFTGS | | | |
| | | TRDTSKN | | | | GSGTDFT | | | |
| | | QVFLKLK | | | | LKISRLE | | | |
| | | SVTTEDT | | | | AEDLGVY | | | |
| | | ATYYCVR | | | | YCWQGTH | | | |
| | | GDQHWGQ | | | | FPQTFGG | | | |
| | | GTALTVS | | | | GTKLEIK | | | |
| | | S | | | | (SEQ ID | | | |
| | | (SEQ ID | | | | NO: 504) | | | |
| | | NO: 500) | | | | | | | |
| ACI- | 917. | QVQLQQP | SYWMH | NVNPNNS | SPYYGGR | DIVMSQS | KSSQSLL | WAFTRES | QQYYSYP |
| 8033- | 7A2B6A9 | GTELVKP | (SEQ ID | DSNYNEK | YLDY | PSSLAVS | YRSNQKN | (SEQ ID | LT |
| 7A2- | | GASVNLP | NO: 311) | FKR | (SEQ ID | VGEKVTM | YLA | NO: 516) | (SEQ ID |
| Ab1 | | CKASGYT | | (SEQ ID | NO: 513) | TCKSSQS | (SEQ ID | | NO: 517) |
| | | FTSYWMH | | NO: 512) | | LLYRSNQ | NO: 515) | | |
| | | WVKQRPG | | | | KNYLAWY | | | |
| | | QGLDWIG | | | | QQKPGQS | | | |
| | | NVNPNNS | | | | PKLLIYW | | | |
| | | DSNYNEK | | | | AFTRESG | | | |
| | | FKRKATL | | | | VPDRFTG | | | |
| | | TVDKSSS | | | | SGSGTDF | | | |
| | | TAYMHLS | | | | TLTISSV | | | |
| | | SLTSEDS | | | | KAEDLAV | | | |
| | | AVYYCAR | | | | YYCQQYY | | | |
| | | SPYYGGR | | | | SYPLTFG | | | |
| | | YLDYWGQ | | | | AGTKLEL | | | |
| | | GTTLTVS | | | | K | | | |
| | | S | | | | (SEQ ID | | | |
| | | (SEQ ID | | | | NO: 514) | | | |
| | | NO: 510) | | | | | | | |
| ACI- | 917. | QVHLKQS | DFYIN | RIYPGNN | GYYGA | DVVMTQT | RSSQSLV | KVSNRFS | SQSTHVP |
| 8033- | 1A12C1B4 | GADLVRP | (SEQ ID | NTFYNEK | (SEQ ID | PLSLPVS | HSNGNTH | (SEQ ID | WT |
| 1A12- | | GASVKLS | NO: 521) | FKG | NO: 463) | LGDQASI | LH | NO: 16) | (SEQ ID |
| Ab1 | | CKASGYS | | (SEQ ID | | SCRSSQS | (SEQ ID | | NO: 467) |
| | | FTDFYIN | | NO: 522) | | LVHSNGN | NO: 525) | | |
| | | WVKQTPG | | | | THLHWYL | | | |
| | | QGLEWIA | | | | QKPGQSP | | | |
| | | RIYPGNN | | | | KLLIYKV | | | |
| | | NTFYNEK | | | | SNRFSGV | | | |
| | | FKGKATL | | | | PDRFSGS | | | |
| | | SAEKSST | | | | GSGTDFT | | | |
| | | TAYMQLS | | | | LKISRVE | | | |
| | | SLTSEDS | | | | AEDLGFY | | | |
| | | AVYFCVV | | | | FCSQSTH | | | |
| | | GYYGAWG | | | | VPWTFGG | | | |
| | | QGTTLTV | | | | GTKLEIK | | | |
| | | SS | | | | (SEQ ID | | | |
| | | (SEQ ID | | | | NO: 524) | | | |
| | | NO: 520) | | | | | | | |
| ACI- | 917. | EVQLQQS | DYYMN | DINPNTG | TGYGDPI | DIVMSQS | KSSQSLL | WASTRES | KQSYNLW |
| 8033- | 4F3F4G6 | GPELVKP | (SEQ ID | TNSYNQK | SSYYYAL | PSSLAVS | NSRTRKN | (SEQ ID | T |
| 4F3- | | GASVKIS | NO: 371) | FKG | DY | AGEKVTM | YLA | NO: 376) | (SEQ ID |
| Ab1 | | CKASGYT | | (SEQ ID | (SEQ ID | SCKSSQS | (SEQ ID | | NO: 537) |
| | | FTDYYMN | | NO: 532) | NO: 533) | LLNSRTR | NO: 345) | | |
| | | WVKQSHG | | | | KNYLAWY | | | |
| | | KSLEWIG | | | | QQKPGQS | | | |
| | | DINPNTG | | | | PKLLIYW | | | |
| | | TNSYNQK | | | | ASTRESG | | | |
| | | FKGRASL | | | | VPDRFTG | | | |
| | | TVDKFSS | | | | SGSGTDF | | | |
| | | AAYMELR | | | | TLTISSV | | | |
| | | SLTSEDS | | | | QAEDLAV | | | |
| | | AVYYCAR | | | | YYCKQSY | | | |
| | | TGYGDPI | | | | NLWTFGG | | | |
| | | SSYYYAL | | | | GTKLEIK | | | |
| | | DYWGQGT | | | | (SEQ ID | | | |
| | | SVTVSS | | | | NO: 534) | | | |
| | | (SEQ ID | | | | | | | |
| | | NO: 530) | | | | | | | |
| ACI- | 917. | EVQLQQS | DYFMN | DINPNID | GRDYAMD | DIVMSQS | KSSQSLL | WASTRES | KQSYDLW |
| 8033- | 17F5F5G9 | GPELVKP | (SEQ ID | VTNYNQK | F | PSSLAVS | NSRTRKN | (SEQ ID | T |
| 17F5- | | GASVKIS | NO: 341) | FKG | (SEQ ID | AGEKVTM | YLA | NO: 376) | (SEQ ID |
| Ab1 | | CKASGYT | | (SEQ ID | NO: 543) | SCKSSQS | (SEQ ID | | NO: 347) |
| | | FTDYFMN | | NO: 542) | | LLNSRTR | NO: 345) | | |
| | | WVKQSHG | | | | KNYLAWY | | | |
| | | KSLEWIG | | | | QQKPGQS | | | |
| | | DINPNID | | | | PKLLIYW | | | |
| | | VTNYNQK | | | | ASTRESG | | | |
| | | FKGKATL | | | | VPDRFTG | | | |
| | | TVDKSSS | | | | SGSGTDF | | | |
| | | TAYMELR | | | | TLTISSV | | | |
| | | SLTSEDS | | | | QAEDLAV | | | |
| | | AVYYCAR | | | | YYCKQSY | | | |
| | | GRDYAMD | | | | DLWTFGG | | | |
| | | FWGQGTS | | | | GTKLEIK | | | |
| | | VTVSS | | | | (SEQ ID | | | |
| | | (SEQ ID | | | | NO: 544) | | | |
| | | NO: 540) | | | | | | | |
| ACI- | 917. | QVQLQQP | SYWIT | DIYPGGG | AQTTFAY | DVLMTQT | RSSQNIV | KVSNRFS | FQGSHVP |
| 8033- | 18C11A11F10 | GAELVKP | (SEQ ID | VTNYNEK | (SEQ ID | PLSLPVS | HNNGNTY | (SEQ ID | RT |
| 18C11- | | GASVKMS | NO: 551) | FKT | NO: 553) | LGDQASI | LE | NO: 16) | (SEQ ID |
| Ab1 | | CKAAGYT | | (SEQ ID | | SCRSSQN | (SEQ ID | | NO: 557) |
| | | FSSYWIT | | NO: 552) | | IVHNNGN | NO: 555) | | |
| | | WVRQRPG | | | | TYLEWYL | | | |
| | | QGLDWIG | | | | QKPGQSP | | | |
| | | DIYPGGG | | | | KLLIYKV | | | |
| | | VTNYNEK | | | | SNRFSGV | | | |
| | | FKTKATL | | | | PDRFSGS | | | |
| | | TVDTSSS | | | | GSGTDFT | | | |
| | | TAYMQLS | | | | LKISRVE | | | |
| | | SLTSEDS | | | | AEDLGVY | | | |
| | | AVYYCAT | | | | YCFQGSH | | | |
| | | AQTTFAY | | | | VPRTFGG | | | |
| | | WGQGTLV | | | | GTKLEIK | | | |
| | | TVSA | | | | (SEQ ID | | | |
| | | (SEQ ID | | | | NO: 554) | | | |
| | | NO: 550) | | | | | | | |
| ACI- | 917. | QVQLQQP | SYWIT | DIYPGGG | AQTTFAH | DVLMTQT | RSSQNIA | KVSNRFS | FQGSHVP |
| 8033- | 18D12F10D6 | GAELVKP | (SEQ ID | VTNYNEK | (SEQ ID | PLSLPVS | HNNGNTY | (SEQ ID | RT |
| 18D12- | | GASVKMS | NO: 551) | FKT | NO: 563) | LGDQASI | LE | NO: 16) | (SEQ ID |
| Ab1 | | CKASGYT | | (SEQ ID | | SCRSSQN | (SEQ ID | | NO: 557) |
| | | FTSYWIT | | NO: 552) | | IAHNNGN | NO: 565) | | |
| | | WVRQRPG | | | | TYLEWYL | | | |
| | | QGLDWIG | | | | QKPGQSP | | | |
| | | DIYPGGG | | | | KLLIYKV | | | |
| | | VTNYNEK | | | | SNRFSGV | | | |
| | | FKTKATL | | | | PDRFSGS | | | |
| | | TVDTSSS | | | | GSGTDFT | | | |
| | | TAYMHLS | | | | LKISRVE | | | |
| | | SLTSEDS | | | | AEDLGVY | | | |
| | | AVYFCAT | | | | YCFQGSH | | | |
| | | AQTTFAH | | | | VPRTFGG | | | |
| | | WGQGTLV | | | | GTKLEIK | | | |
| | | TVSA | | | | (SEQ ID | | | |
| | | (SEQ ID | | | | NO: 564) | | | |
| | | NO: 560) | | | | | | | |
| ACI- | 917. | DVQLQES | SGFYWN | YISYDGS | GDVD | DVVMTQT | KSSQSLL | LVSKLDS | WQGTHFP |
| 8033- | 1F8D8E4 | GPGLVKP | (SEQ ID | NNYNPSL | (SEQ ID | PLTLSVT | DSDGETY | (SEQ ID | QT |
| 1F8- | | SQSLSLT | NO: 571) | KN | NO: 573) | IGQPASI | LN | NO: 106) | (SEQ ID |
| Ab1 | | CSVTGYS | | (SEQ ID | | SCKSSQS | (SEQ ID | | NO: 107) |
| | | ITSGFYW | | NO: 202) | | LLDSDGE | NO: 105) | | |
| | | NWIRQFP | | | | TYLNWLF | | | |
| | | GNKLEWM | | | | QRPGQSP | | | |
| | | GYISYDG | | | | KRLIYLV | | | |
| | | SNNYNPS | | | | SKLDSGV | | | |
| | | LKNRISI | | | | PDRFTGS | | | |
| | | IRDTSKN | | | | GSGTDFT | | | |
| | | QFFLKLK | | | | LKISRVE | | | |
| | | SVTSEDT | | | | PEDLGVY | | | |
| | | ATYYCVR | | | | YCWQGTH | | | |
| | | GDVDWGQ | | | | FPQTLGG | | | |
| | | GTTLTVS | | | | GTKLEIK | | | |
| | | S | | | | (SEQ ID | | | |
| | | (SEQ ID | | | | NO: 574) | | | |
| | | NO: 570) | | | | | | | |
| ACI- | 917. | EVQLVES | SYGMS | TISNGGS | QLRRDGW | EIVLTQS | SVSSSIS | GTSNLAS | QQWSSYP |
| 8033- | 22E5C5F7 | GGDLVKP | (SEQ ID | YTYYPDS | YFDV | PALMAAS | SSKLH | (SEQ ID | LT |
| 22E5- | | GGSLKLS | NO: 581) | VKG | (SEQ ID | PGEKVTI | (SEQ ID | NO: 586) | (SEQ ID |
| Ab1 | | CAASGFT | | (SEQ ID | NO: 583) | TCSVSSS | NO: 585) | | NO: 587) |
| | | FSSYGMS | | NO: 582) | | ISSSKLH | | | |
| | | WVRQTPD | | | | WYQQKSE | | | |
| | | KRLEWVA | | | | TSPKLWI | | | |
| | | TISNGGS | | | | YGTSNLA | | | |
| | | YTYYPDS | | | | SGVPVRF | | | |
| | | VKGRFTI | | | | SGSGSGT | | | |
| | | SRDNAKN | | | | SYSLTIS | | | |
| | | TLYLQMS | | | | SMEAEDA | | | |
| | | SLKSEDT | | | | ATYYCQQ | | | |
| | | AMYYCAR | | | | WSSYPLT | | | |
| | | QLRRDGW | | | | FGAGTKL | | | |
| | | YFDVWGT | | | | ELK | | | |
| | | GTTVTVS | | | | (SEQ ID | | | |
| | | S | | | | NO: 584) | | | |
| | | (SEQ ID | | | | | | | |
| | | NO: 580) | | | | | | | |
| ACI- | 917. | EVQLVES | TYAMN | RIRSKSN | SFDY | DIKMTQS | KASQDIN | RAKRLVD | LQYDEFP |
| 8033- | 27D8E1H10E10 | GGGLVQP | (SEQ ID | NFATYYA | (SEQ ID | PSSMYAS | SYLS | (SEQ ID | FT |
| 27D8- | | KGSLKLS | NO: 141) | DSVKD | NO: 473) | LGERVTI | (SEQ ID | NO: 476) | (SEQ ID |
| Ab1 | | CAASGFT | | (SEQ ID | | TCKASQD | NO: 475) | | NO: 477) |
| | | FNTYAMN | | NO: 472) | | INSYLSW | | | |
| | | WVRQAPG | | | | FQQKPGK | | | |
| | | KGLEWVA | | | | SPKTLIY | | | |
| | | RIRSKSN | | | | RAKRLVD | | | |
| | | NFATYYA | | | | GVPSRFS | | | |
| | | DSVKDRF | | | | GSGSGQD | | | |
| | | TISRDES | | | | YSLTISS | | | |
| | | ESMLYLQ | | | | LEYEDMG | | | |
| | | MNNLKAE | | | | IYYCLQY | | | |
| | | DTAMYYC | | | | DEFPFTF | | | |
| | | VRSFDYW | | | | GSGTKLE | | | |
| | | GQGTTLT | | | | IK | | | |
| | | VSS | | | | (SEQ ID | | | |
| | | (SEQ ID | | | | NO: 474) | | | |
| | | NO: 590) | | | | | | | |
| ACI- | 917. | QVQLQQP | SYWIT | DIYPGGG | AQTTFAY | DVLMTQT | RSSQNIV | KVSNRFS | FQGSHVP |
| 8033- | 21C8E4C8 | GAELVKP | (SEQ ID | VTNYNEK | (SEQ ID | PLSLPVS | HNNGNTY | (SEQ ID | RT |
| 21C8- | | GASVKMS | NO: 551) | FKT | NO: 553) | LGDQASI | LE | NO: 16) | (SEQ ID |
| Ab1 | | CKASGYT | | (SEQ ID | | SCRSSQN | (SEQ ID | | NO: 557) |
| | | FTSYWIT | | NO: 552) | | IVHNNGN | NO: 555) | | |
| | | WVRQRPG | | | | TYLEWYL | | | |
| | | QGLDWIG | | | | QKPGQSP | | | |
| | | DIYPGGG | | | | KLLIYKV | | | |
| | | VTNYNEK | | | | SNRFSGV | | | |
| | | FKTKATL | | | | PDRFSGS | | | |
| | | TVDTSSS | | | | GSGTDFT | | | |
| | | TAYMHLS | | | | LKISRVE | | | |
| | | SLTSEDS | | | | AEDLGVY | | | |
| | | AVYFCAT | | | | YCFQGSH | | | |
| | | AQTTFAY | | | | VPRTFGG | | | |
| | | WGQGTLV | | | | GTKLEIK | | | |
| | | TVSA | | | | (SEQ ID | | | |
| | | (SEQ ID | | | | NO: 554) | | | |
| | | NO: 600) | | | | | | | |
| ACI- | 3101E3C9 | QVQLKES | RYIIN | VIWTGGG | EYGYDGA | DVLMTQT | RSSQTIV | KVSNRFS | FQGSHVP |
| 7079- | | GPGLVAP | (SEQ ID | TDYNSAL | WFAY | PLSLPVS | HSNGNTY | (SEQ ID | YT |
| 3101E3- | | SQSLSIT | NO: 611) | KS | (SEQ ID | LGDQASI | LE | NO: 616) | (SEQ ID |
| Ab1 | | CTVSGFS | | (SEQ ID | NO: 613) | SCRSSQT | (SEQ ID | | NO: 617) |
| | | LTRYIIN | | NO: 612) | | IVHSNGN | NO: 615) | | |
| | | WVRQPPG | | | | TYLEWYL | | | |
| | | KGLEWLG | | | | QKPGQSP | | | |
| | | VIWTGGG | | | | KLLIYKV | | | |
| | | TDYNSAL | | | | SNRFSGV | | | |
| | | KSRLSIS | | | | PDRFSGS | | | |
| | | KDNSKSQ | | | | GSGTDFT | | | |
| | | VFLKMNS | | | | LKISRVE | | | |
| | | LQTDDTA | | | | AEDLGVY | | | |
| | | RYYCARE | | | | YCFQGSH | | | |
| | | YGYDGAW | | | | VPYTFGG | | | |
| | | FAYWGQG | | | | GTKLEIK | | | |
| | | TLVTVSA | | | | (SEQ ID | | | |
| | | (SEQ ID | | | | NO: 614) | | | |
| | | NO: 610) | | | | | | | |
| ACI- | 3103D9C9 | QVSLKES | TFGMGVG | HIWWDDD | IGDYYGT | DIVMTQS | KSSQSLL | FASTRES | QQHYSTP |
| 7079- | | GPGILQP | (SEQ ID | KYYNPGL | RGYFDV | PSSLTMS | NSSNQKN | (SEQ ID | YT |
| 3103D9- | | SQTLSLT | NO: 621) | KS | (SEQ ID | LGQTVTM | HLA | NO: 626) | (SEQ ID |
| Ab1 | | CSFSGFS | | (SEQ ID | NO: 623) | SCKSSQS | (SEQ ID | | NO: 627) |
| | | LSTFGMG | | NO: 622) | | LLNSSNQ | NO: 625) | | |
| | | VGWIRQP | | | | KNHLAWY | | | |
| | | SGKGLDW | | | | QQRPGQS | | | |
| | | LTHIWWD | | | | PKLLVYF | | | |
| | | DDKYYNP | | | | ASTRESG | | | |
| | | GLKSRLT | | | | VPERFIG | | | |
| | | ISKDTSK | | | | GGSGTDF | | | |
| | | NQVFLEI | | | | TLTISNV | | | |
| | | ANVDTAD | | | | QSEDLTD | | | |
| | | TATYYCA | | | | YFCQQHY | | | |
| | | RIGDYYG | | | | STPYTFG | | | |
| | | TRGYFDV | | | | GGTKLEI | | | |
| | | WGTGTTV | | | | K | | | |
| | | TVSS | | | | (SEQ ID | | | |
| | | (SEQ ID | | | | NO: 624) | | | |
| | | NO: 620) | | | | | | | |
| ACI- | 3103G12 | DVQLQES | SGYDWH | YITYSGS | DTVDAWF | DVLMTQA | RSSQSIV | KVSNRFS | FQGSHVP |
| 7079- | D2B10 | GPGMVKP | (SEQ ID | TNYNPSL | AY | PLSLPVS | HSNGDTY | (SEQ ID | PT |
| 3103G12- | | SQSLSLT | NO: 631) | KS | (SEQ ID | LGDQASI | LE | NO: 616) | (SEQ ID |
| Ab2 | | CTVTGYS | | (SEQ ID | NO: 633) | SCRSSQS | (SEQ ID | | NO: 637) |
| | | ITSGYDW | | NO: 632) | | IVHSNGD | NO: 635) | | |
| | | HWIRHFP | | | | TYLEWYL | | | |
| | | GNKLEWM | | | | QKPGQSP | | | |
| | | GYITYSG | | | | KLLIYKV | | | |
| | | STNYNPS | | | | SNRFSGV | | | |
| | | LKSRISI | | | | PDRFSGS | | | |
| | | THDTSKN | | | | GSGTDFT | | | |
| | | HFFLKLT | | | | LKISRVE | | | |
| | | SVTTEDT | | | | AEDLGVY | | | |
| | | ATYYCAR | | | | YCFQGSH | | | |
| | | DTVDAWF | | | | VPPTFGG | | | |
| | | AYWGQGT | | | | GTKLEIK | | | |
| | | LVTVSA | | | | (SEQ ID | | | |
| | | (SEQ ID | | | | NO: 634) | | | |
| | | NO: 630) | | | | | | | |
| ACI- | 3104F12F2H7 | QVTLKES | TFGLGVG | HIWWDDD | IGDFYGS | DIVMTQS | KSSQSLL | FASTRES | QQHYSTP |
| 7079- | | GPGIVQP | (SEQ ID | KYYNPAL | RGYFDV | PSSLAMS | NSSNQKN | (SEQ ID | YT |
| 3104F12- | | SQTLSLT | NO: 641) | KS | (SEQ ID | VGQKVTL | HLA | NO: 626) | (SEQ ID |
| Ab2 | | CSFSGFS | | (SEQ ID | NO: 643) | RCKSSQS | (SEQ ID | | NO: 627) |
| | | LNTFGLG | | NO: 642) | | LLNSSNQ | NO: 625) | | |
| | | VGWIRQP | | | | KNHLAWY | | | |
| | | SGKGLDW | | | | QQKPGQS | | | |
| | | LTHIWWD | | | | PKLLLCF | | | |
| | | DDKYYNP | | | | ASTRESG | | | |
| | | ALKSRLT | | | | VPDRFIG | | | |
| | | ISKDTSK | | | | SGSGTDF | | | |
| | | NQVFLKI | | | | TLTISSV | | | |
| | | ANVDTAD | | | | QAEDLAD | | | |
| | | TATYYCA | | | | YFCQQHY | | | |
| | | RIGDFYG | | | | STPYTFG | | | |
| | | SRGYFDV | | | | GGTKLEI | | | |
| | | WGTGTTV | | | | K | | | |
| | | TVSS | | | | (SEQ ID | | | |
| | | (SEQ ID | | | | NO: 644) | | | |
| | | NO: 640) | | | | | | | |
| ACI- | 3106C5B7 | QVTLKES | TFGMGVG | HIWWDDD | IGDYYGS | DIVMTQS | KSRQSLL | FASTRES | QQHYSTP |
| 7079- | | GPGILQP | (SEQ ID | KYYNPAL | RGYFDV | PSSLAMS | NSSNQKN | (SEQ ID | YT |
| 3106C5- | | SQTLSLT | NO: 621) | KS | (SEQ ID | VGQKVTM | HLA | NO: 626) | (SEQ ID |
| Ab1 | | CSFSGFS | | (SEQ ID | NO: 653) | SCKSRQS | (SEQ ID | | NO: 627) |
| | | LSTFGMG | | NO: 642) | | LLNSSNQ | NO: 655) | | |
| | | VGWIRQP | | | | KNHLAWY | | | |
| | | SGKGLEW | | | | QQKPGQS | | | |
| | | LTHIWWD | | | | PKLLVYF | | | |
| | | DDKYYNP | | | | ASTRESG | | | |
| | | ALKSRLT | | | | VPDRFIG | | | |
| | | ISRDPSK | | | | SGSGTDF | | | |
| | | NQVFLKI | | | | TLTISSV | | | |
| | | ANVDTAD | | | | QAEDLAD | | | |
| | | TATYYCA | | | | YFCQQHY | | | |
| | | RIGDYYG | | | | STPYTFG | | | |
| | | SRGYFDV | | | | GGTKLEI | | | |
| | | WGTGTTV | | | | K | | | |
| | | TVSS | | | | (SEQ ID | | | |
| | | (SEQ ID | | | | NO: 654) | | | |
| | | NO: 650) | | | | | | | |
| ACI- | 3106F2C8 | EVQLQQS | DDYMH | WIDPENG | AGTSPYY | DIVMTQS | KASQNVG | SASYRYS | QQYNSYP |
| 7079- | | GAELVRP | (SEQ ID | DTEYASK | FDY | QKFMSTS | TNVA | (SEQ ID | LT |
| 3106F2- | | GASVKLS | NO: 661) | FQG | (SEQ ID | VGDRVSV | (SEQ ID | NO: 666) | (SEQ ID |
| Ab1 | | CTASGFN | | (SEQ ID | NO: 663) | TCKASQN | NO: 665) | | NO: 667) |
| | | VKDDYMH | | NO: 662) | | VGTNVAW | | | |
| | | WVKQRPE | | | | YQQKVGQ | | | |
| | | QGLEWIG | | | | SPKALIY | | | |
| | | WIDPENG | | | | SASYRYS | | | |
| | | DTEYASK | | | | GVPDRFT | | | |
| | | FQGKATL | | | | GSGSGTD | | | |
| | | TADTSSN | | | | FTLTISN | | | |
| | | TAYLQLS | | | | VQSEDLA | | | |
| | | GLTSVDT | | | | DYFCQQY | | | |
| | | AVYYCTT | | | | NSYPLTF | | | |
| | | AGTSPYY | | | | GSGTKLE | | | |
| | | FDYWGQG | | | | IK | | | |
| | | TTLTVSS | | | | (SEQ ID | | | |
| | | (SEQ ID | | | | NO: 664) | | | |
| | | NO: 660) | | | | | | | |
| ACI- | 3112H1F3 | EVQLQQS | DYFMN | DINPNNG | GSNYAMD | DIVMSQS | KSSQSLL | WASTRES | KQSYDLW |
| 7079- | | GPELVKP | (SEQ ID | GSSYIQK | S | PSSLAVS | NSRTRKN | (SEQ ID | T |
| 3112H1- | | GASVKIS | NO: 671) | FKG | (SEQ ID | AGEKVTM | YLA | NO: 676) | (SEQ ID |
| Ab1 | | CKASGYT | | (SEQ ID | NO: 673) | SCKSSQS | (SEQ ID | | NO: 677) |
| | | FSDYFMN | | NO: 672) | | LLNSRTR | NO: 675) | | |
| | | WVKQSHG | | | | KNYLAWY | | | |
| | | KSLEWIG | | | | QQKPGQS | | | |
| | | DINPNNG | | | | PKLLIYW | | | |
| | | GSSYIQK | | | | ASTRESG | | | |
| | | FKGKATL | | | | VPDRFTG | | | |
| | | TVDKSST | | | | SGSGTDF | | | |
| | | TAYMELR | | | | TLTISSV | | | |
| | | SLTSEDS | | | | QAEDLAV | | | |
| | | AVYYCAR | | | | YYCKQSY | | | |
| | | GSNYAMD | | | | DLWTFGG | | | |
| | | SWGQGTS | | | | GTKLEIK | | | |
| | | VTVSS | | | | (SEQ ID | | | |
| | | (SEQ ID | | | | NO: 674) | | | |
| | | NO: 670) | | | | | | | |
| | | QVTLKES | TFGMGVG | HIWWDDD | IGDYYGS | DIVMTQS | KSSQSLL | FASTRDS | QQHYSTP |
| | | GPGILQP | (SEQ ID | KYYNPAL | RGFFDV | PSSLAMS | NSSNQKN | (SEQ ID | YT |
| | | SQTLSLT | NO: 621) | KS | (SEQ ID | VGQKVTM | HLA | NO: 686) | (SEQ ID |
| | | CSFSGFS | | (SEQ ID | NO: 683) | SCKSSQS | (SEQ ID | | NO: 687) |
| | | LSTFGMG | | NO: 642) | | LLNSSNQ | NO: 625) | | |
| | | VGWIRQP | | | | KNHLAWY | | | |
| | | SGKGLEW | | | | QQKPGQS | | | |
| | | LTHIWWD | | | | PKLLVYF | | | |
| | | DDKYYNP | | | | ASTRDSG | | | |
| | | ALKSRLT | | | | VPDRFIG | | | |
| | | ISKDASK | | | | SGSGTDF | | | |
| | | NQMFLKI | | | | TLTISSV | | | |
| | | ANVDTAD | | | | QAEDLAD | | | |
| | | TATYFCA | | | | YFCQQHY | | | |
| | | RIGDYYG | | | | STPYTFG | | | |
| | | SRGFFDV | | | | GGTKLEI | | | |
| | | WGTGTTV | | | | K | | | |
| ACI- | 3107E6A3 | TVSS | | | | (SEQ ID | | | |
| 7079- | | (SEQ ID | | | | NO: 684) | | | |
| 3107E6- | | NO: 680) | | | | | | | |
| Ab1 | | | | | | | | | |
| ACI- | 3108C10G6 | ELQLVES | NYAMS | TISDGGT | AGSGH | DVVMTQT | KSSQSLL | LVSKVDS | WQGTYFP |
| 7079- | | GGGLVRP | (SEQ ID | YTFYSDN | (SEQ ID | PRTLSVI | DSDGKTY | (SEQ ID | RT |
| 3108C10- | | GGSLKLS | NO: 691) | VKG | NO: 693) | IGQPASI | LS | NO: 696) | (SEQ ID |
| Ab2 | | CATSGFT | | (SEQ ID | | SCKSSQS | (SEQ ID | | NO: 697) |
| | | FRNYAMS | | NO: 692) | | LLDSDGK | NO: 695) | | |
| | | WVRQTPA | | | | TYLSWLL | | | |
| | | KRLEWVA | | | | QRPGQSP | | | |
| | | TISDGGT | | | | KRLIYLV | | | |
| | | YTFYSDN | | | | SKVDSGV | | | |
| | | VKGRFTI | | | | PDRFTGS | | | |
| | | SRDNVKN | | | | GSGTDFT | | | |
| | | TLYLQMS | | | | LKISRVE | | | |
| | | HLKSEDT | | | | AEDLGVY | | | |
| | | AMYYCSR | | | | YCWQGTY | | | |
| | | AGSGHWG | | | | FPRTFGG | | | |
| | | QGTTLTV | | | | GTKLEIK | | | |
| | | SS | | | | (SEQ ID | | | |
| | | (SEQ ID | | | | NO: 694) | | | |
| | | NO: 690) | | | | | | | |
| ACI- | 6106F5B10 | QVQLQQS | TYYIH | WIYPGRG | DWETGFP | DIVLTQS | KASQSVD | AASNLDS | QQSGEDP |
| 8030- | | GPELVKP | (SEQ ID | YTKYNEK | Y | PASLAVS | YGGDSWL | (SEQ ID | FT |
| 6106F5- | | GASVKIS | NO: 701) | FKD | (SEQ ID | LGQRATI | N | NO: 706) | (SEQ ID |
| Ab1 | | CKASGYS | | (SEQ ID | NO: 703) | SCKASQS | (SEQ ID | | NO: 707) |
| | | FTTYYIH | | NO: 702) | | VDYGGDS | NO: 705) | | |
| | | WMKQRPG | | | | WLNWYQQ | | | |
| | | QGPEWIG | | | | KPGQPPK | | | |
| | | WIYPGRG | | | | LLIYAAS | | | |
| | | YTKYNEK | | | | NLDSGIP | | | |
| | | FKDKAAQ | | | | ARFGGSG | | | |
| | | TADTSSN | | | | SGTDFTL | | | |
| | | TAYMQLS | | | | NIHPVEE | | | |
| | | SLRSEDS | | | | EDAATYY | | | |
| | | AVYYCAR | | | | CQQSGED | | | |
| | | DWETGFP | | | | PFTFGGG | | | |
| | | YWGQGTL | | | | TNLEIK | | | |
| | | VTVSA | | | | (SEQ ID | | | |
| | | (SEQ ID | | | | NO: 704) | | | |
| | | NO: 700) | | | | | | | |
| ACI- | 6207G10E6 | QVQLQQS | SYWMN | QIYPGDG | THYDY | DILLTQS | RASQSIG | YASESIS | QQSNSWP |
| 8031- | | GAELVKP | (SEQ ID | DTNYNGK | (SEQ ID | PAILSVS | TRIH | (SEQ ID | LT |
| 6207G10- | | GASVKIS | NO: 711) | FKG | NO: 713) | PGDRVSF | (SEQ ID | NO: 716) | (SEQ ID |
| Ab1 | | CKASGYA | | (SEQ ID | | SCRASQS | NO: 715) | | NO: 717) |
| | | FSSYWMN | | NO: 712) | | IGTRIHW | | | |
| | | WVKQRPG | | | | YQQRTNG | | | |
| | | KGLEWIG | | | | SPRLLIK | | | |
| | | QIYPGDG | | | | YASESIS | | | |
| | | DTNYNGK | | | | GTPSRFS | | | |
| | | FKGKATL | | | | GSGSGTD | | | |
| | | TADRSSS | | | | FTLSINS | | | |
| | | TAYMQLS | | | | VESEDIA | | | |
| | | SLTSEDS | | | | DYYCQQS | | | |
| | | AVYFCAI | | | | NSWPLTF | | | |
| | | THYDYWG | | | | GAGTKLE | | | |
| | | QGTTLTV | | | | LK | | | |
| | | SS | | | | (SEQ ID | | | |
| | | (SEQ ID | | | | NO: 714) | | | |
| | | NO: 710) | | | | | | | |
| ACI- | 6301A10E12 | QVQLQQS | SYGIS | EIYPRSG | ESYYGDY | DVLMTQT | RSSHSIE | KVSNRFS | FQGSHVP |
| 8032- | | GAELARP | (SEQ ID | NTYYNEK | DWYFDV | PLSLPVS | HSNGNTY | (SEQ ID | PT |
| 6301A10- | | GASVKLS | NO: 721) | FKG | (SEQ ID | LGDQASI | LE | NO: 616) | (SEQ ID |
| Ab2 | | CKASGYT | | (SEQ ID | NO: 723) | SCRSSHS | (SEQ ID | | NO: 637) |
| | | FTSYGIS | | NO: 722) | | IEHSNGN | NO: 725) | | |
| | | WVKQRTG | | | | TYLEWCL | | | |
| | | QGLEWIG | | | | QKPGQPP | | | |
| | | EIYPRSG | | | | KFLIYKV | | | |
| | | NTYYNEK | | | | SNRFSGV | | | |
| | | FKGKATL | | | | PDRFSGS | | | |
| | | TADKSSS | | | | GSGTDFT | | | |
| | | TAYMELR | | | | LRISRVE | | | |
| | | SLTSEDS | | | | AEDLGVY | | | |
| | | AVYFCAS | | | | YCFQGSH | | | |
| | | ESYYGDY | | | | VPPTFGG | | | |
| | | DWYFDVW | | | | GTKLEIK | | | |
| | | GTGTTVT | | | | (SEQ ID | | | |
| | | VSS | | | | NO: 724) | | | |
| | | (SEQ ID | | | | | | | |
| | | NO: 720) | | | | | | | |
| ACI- | 6301C8G12 | QVQLQQS | TYGIS | EIYPRSG | ESYYGDY | DVLMTQI | RSSQSIV | EVSNRFS | FQGSHVP |
| 8032- | | GAELARP | (SEQ ID | NTYYNEK | AWYFDV | PLSLPVS | HSNGNTY | (SEQ ID | PT |
| 6301C8- | | GTSVKLS | NO: 731) | FKG | (SEQ ID | LGDQASI | LE | NO: 736) | (SEQ ID |
| Ab2 | | CKASGYT | | (SEQ ID | NO: 733) | SCRSSQS | (SEQ ID | | NO: 637) |
| | | FTTYGIS | | NO: 722) | | IVHSNGN | NO: 735) | | |
| | | WVKQRTG | | | | TYLEWHL | | | |
| | | QGLEWIG | | | | QKPGQSP | | | |
| | | EIYPRSG | | | | KLLIYEV | | | |
| | | NTYYNEK | | | | SNRFSGV | | | |
| | | FKGKATL | | | | PDRFSGS | | | |
| | | TADKPSS | | | | GSGTDFT | | | |
| | | TAYMELR | | | | LNISRVE | | | |
| | | SLTSEDS | | | | AEDLGVY | | | |
| | | AVYFCAS | | | | YCFQGSH | | | |
| | | ESYYGDY | | | | VPPTFGG | | | |
| | | AWYFDVW | | | | GTKLEIK | | | |
| | | GTGTTVT | | | | (SEQ ID | | | |
| | | VSS | | | | NO: 734) | | | |
| | | (SEQ ID | | | | | | | |
| | | NO: 730) | | | | | | | |
| ACI- | 6301G2D11 | EVQLQQS | DYFMN | DINPNIG | GGGSNPL | DIVMSQS | KSSQSLL | WASTRES | KQSYDLW |
| 8032- | | GPELVKP | (SEQ ID | VTNYNPK | YYAMDY | PSSLAVS | NSRTRKN | (SEQ ID | T |
| 6301G2- | | GASVKIS | NO: 671) | FKD | (SEQ ID | AGEKVTM | YLA | NO: 676) | (SEQ ID |
| Ab2 | | CKASGYT | | (SEQ ID | NO: 743) | SCKSSQS | (SEQ ID | | NO: 677) |
| | | FTDYFMN | | NO: 742) | | LLNSRTR | NO: 675) | | |
| | | WVKQSHG | | | | KNYLAWY | | | |
| | | KSLEWIG | | | | QQKPGQS | | | |
| | | DINPNIG | | | | PKLLIYW | | | |
| | | VTNYNPK | | | | ASTRESG | | | |
| | | FKDRATL | | | | VPDRFTG | | | |
| | | TVDKSSS | | | | SGSGTDF | | | |
| | | SAYMELR | | | | TLTISSV | | | |
| | | SLTSEDS | | | | QAEDLAV | | | |
| | | TVYYCAR | | | | YFCKQSY | | | |
| | | GGGSNPL | | | | DLWTFGG | | | |
| | | YYAMDYW | | | | GTKLEIK | | | |
| | | GQGTSVT | | | | (SEQ ID | | | |
| | | VSS | | | | NO: 744) | | | |
| | | (SEQ ID | | | | | | | |
| | | NO: 740) | | | | | | | |
| ACI- | 6304F3D12 | QVQLQQS | SYGVS | EIYPRSG | ESYYGDY | DVLMTQI | RSSQSIV | KVSNRFS | FQGSHVP |
| 8032- | | GAELARP | (SEQ ID | NTYYNEK | DWYFDV | PLSLPVS | HSNGNTY | (SEQ ID | PT |
| 6304F3- | | GASVKLS | NO: 751) | FKG | (SEQ ID | PGDQVSI | LE | NO: 616) | (SEQ ID |
| Ab1 | | CKASGYT | | (SEQ ID | NO: 723) | SCRSSQS | (SEQ ID | | NO: 637) |
| | | FTSYGVS | | NO: 722) | | IVHSNGN | NO: 735) | | |
| | | WVKQRTG | | | | TYLEWYL | | | |
| | | QGLEWIG | | | | QKPGQSP | | | |
| | | EIYPRSG | | | | KPLIYKV | | | |
| | | NTYYNEK | | | | SNRFSGV | | | |
| | | FKGKATL | | | | PDRFSGS | | | |
| | | TADKSSS | | | | GSGTDFT | | | |
| | | TAYMELR | | | | LKISRVE | | | |
| | | SLTSEDS | | | | AEDLGVY | | | |
| | | AVYFCAS | | | | YCFQGSH | | | |
| | | ESYYGDY | | | | VPPTFGG | | | |
| | | DWYFDVW | | | | GTKLEIK | | | |
| | | GTGTTVT | | | | (SEQ ID | | | |
| | | VSS | | | | NO: 754) | | | |
| | | (SEQ ID | | | | | | | |
| | | NO: 750) | | | | | | | |
| ACI- | 6307F1H10 | QVQLQQS | SYGMS | EIYPRSG | ESYYGDY | DVLMTQT | RSSQSVV | KVSNRFS | FQGSHVP |
| 8032- | | GAELARP | (SEQ ID | NTYYNEK | AWYFDV | PLSLPVS | HSNGITY | (SEQ ID | PT |
| 6307F1- | | GASVKLS | NO: 761) | FKG | (SEQ ID | LGDQASI | LE | NO: 616) | (SEQ ID |
| Ab2 | | CKASGYT | | (SEQ ID | NO: 733) | SCRSSQS | (SEQ ID | | NO: 637) |
| | | FTSYGMS | | NO: 722) | | VVHSNGI | NO: 765) | | |
| | | WVKQRTG | | | | TYLEWYL | | | |
| | | QGLEWIG | | | | QKPGQSP | | | |
| | | EIYPRSG | | | | KLLIYKV | | | |
| | | NTYYNEK | | | | SNRFSGV | | | |
| | | FKGKATL | | | | PDRFSGS | | | |
| | | TSDKSSS | | | | GSGTYFT | | | |
| | | TSYMELR | | | | LKISRVE | | | |
| | | SLTSEDS | | | | AEDLGVY | | | |
| | | AVYFCAS | | | | YCFQGSH | | | |
| | | ESYYGDY | | | | VPPTFGG | | | |
| | | AWYFDVW | | | | GTKLEIK | | | |
| | | GTGTTVT | | | | (SEQ ID | | | |
| | | VSS | | | | NO: 764) | | | |
| | | (SEQ ID | | | | | | | |
| | | NO: 760) | | | | | | | |
| ACI- | 6313G2A10 | EVQLQQS | DYYMN | DIDPNNG | TGYYGTS | DIVMSQS | KSSQSLL | WASTRES | KQSYDLW |
| 8032- | | GPELVKP | (SEQ ID | VTSYSQK | LYYAMDY | PSSLAVS | NSRTRKN | (SEQ ID | T |
| 6313G2- | | GASVKIS | NO: 771) | FKG | (SEQ ID | AGEKVTM | YLA | NO: 676) | (SEQ ID |
| Ab1 | | CKASGYT | | (SEQ ID | NO: 773) | NCKSSQS | (SEQ ID | | NO: 677) |
| | | FTDYYMN | | NO: 772) | | LLNSRTR | NO: 675) | | |
| | | WVKQSHG | | | | KNYLAWY | | | |
| | | KSLEWIG | | | | QQKPGQS | | | |
| | | DIDPNNG | | | | PKLLIYW | | | |
| | | VTSYSQK | | | | ASTRESG | | | |
| | | FKGKATL | | | | VPDRFIG | | | |
| | | TVDKSSS | | | | SGSGTGF | | | |
| | | TAYMELR | | | | TLTISSV | | | |
| | | SLTSEDS | | | | QAEDLAV | | | |
| | | AVYYCAR | | | | YYCKQSY | | | |
| | | TGYYGTS | | | | DLWTFGG | | | |
| | | LYYAMDY | | | | GTKLEIK | | | |
| | | WGQGTSV | | | | (SEQ ID | | | |
| | | TVSS | | | | NO: 774) | | | |
| | | (SEQ ID | | | | | | | |
| | | NO: 770) | | | | | | | |
| ACI- | 6314A3E4 | QVQLQQS | SYGVS | EIYPRSG | ESYYGDY | DVLMTQI | RSSQSIV | KVSNRFS | FQGSHVP |
| 8032- | | GAELARP | (SEQ ID | NTYYNEK | DWYFDV | PLSLPVS | HSNGNTY | (SEQ ID | PT |
| 6314A3- | | GASVKLS | NO: 751) | FKG | (SEQ ID | PGDQVFI | LE | NO: 616) | (SEQ ID |
| Ab3 | | CKASGYT | | (SEQ ID | NO: 753) | SCRSSQS | (SEQ ID | | NO: 637) |
| | | FTSYGVS | | NO: 752) | | IVHSNGN | NO: 735) | | |
| | | WVKQRTG | | | | TYLEWYL | | | |
| | | QGLEWIG | | | | QKPGQSP | | | |
| | | EIYPRSG | | | | KPLIYKV | | | |
| | | NTYYNEK | | | | SNRFSGV | | | |
| | | FKGKATL | | | | PDRFSGS | | | |
| | | TADKSSS | | | | GSGTDFT | | | |
| | | TAYMELR | | | | LKISRVE | | | |
| | | SLTSEDS | | | | AEDLGVY | | | |
| | | AVYFCAS | | | | YCFQGSH | | | |
| | | ESYYGDY | | | | VPPTFGG | | | |
| | | DWYFDVW | | | | GTKLEIK | | | |
| | | GTGTTVT | | | | (SEQ ID | | | |
| | | VSS | | | | NO: 784) | | | |
| | | (SEQ ID | | | | | | | |
| | | NO: 750) | | | | | | | |
| ACI- | 6401F2E3 | QVQLQQP | NYWIT | DIYPGNG | GQTSFGH | DVLMTQT | RSSQSVV | KVSNRFS | FQGSHVP |
| 8033- | | GAELVKP | (SEQ ID | DVDYSEI | (SEQ ID | PLSLPVS | HVNGNTY | (SEQ ID | RT |
| 6401F2- | | GASVKMS | NO: 791) | FKN | NO: 793) | LGDQASI | LD | NO: 616) | (SEQ ID |
| Ab1 | | CKASGYT | | (SEQ ID | | SCRSSQS | (SEQ ID | | NO: 797) |
| | | FTNYWIT | | NO: 792) | | VVHVNGN | NO: 795) | | |
| | | WVKQRPG | | | | TYLDWYL | | | |
| | | QGLEWIG | | | | QKPGQSP | | | |
| | | DIYPGNG | | | | KLLIYKV | | | |
| | | DVDYSEI | | | | SNRFSGV | | | |
| | | FKNKAKM | | | | PDRFSGS | | | |
| | | TVDTSST | | | | GSGTDFT | | | |
| | | TAYMQLS | | | | LRITRVE | | | |
| | | SLTSEDS | | | | AEDLGIY | | | |
| | | AVYHCVL | | | | YCFQGSH | | | |
| | | GQTSFGH | | | | VPRTFGG | | | |
| | | WGQGTLV | | | | GTKLEIK | | | |
| | | TVSA | | | | (SEQ ID | | | |
| | | (SEQ ID | | | | NO: 794) | | | |
| | | NO: 790) | | | | | | | |
| ACI- | 6402E2E4 | EVQLQHS | DYYMN | DINPNNG | SGSNYYA | DIVMSQS | KSSQSLF | WASTRES | KQSYDLW |
| 8033- | | GPDLVKP | (SEQ ID | DTTYNQK | MDS | PSSLAVS | NSRTRKN | (SEQ ID | T |
| 6402E2- | | GASVKIS | NO: 771) | FKG | (SEQ ID | AGERVTM | YLA | NO: 676) | (SEQ ID |
| Ab2 | | CKASGYT | | (SEQ ID | NO: 803) | SCKSSQS | (SEQ ID | | NO: 677) |
| | | FTDYYMN | | NO: 802) | | LFNSRTR | NO: 805) | | |
| | | WVKQSHG | | | | KNYLAWY | | | |
| | | KSLEWIG | | | | QQKPGQS | | | |
| | | DINPNNG | | | | PKLLIYW | | | |
| | | DTTYNQK | | | | ASTRESG | | | |
| | | FKGRATL | | | | VPDRFTG | | | |
| | | TVDKSSS | | | | SGSGTDF | | | |
| | | TAYMELR | | | | SLNISSV | | | |
| | | SLTSEDS | | | | QAEDLAV | | | |
| | | AVYYCAR | | | | YYCKQSY | | | |
| | | SGSNYYA | | | | DLWTFGG | | | |
| | | MDSWGQG | | | | GTKLEIK | | | |
| | | TSVTVSS | | | | (SEQ ID | | | |
| | | (SEQ ID | | | | NO: 804) | | | |
| | | NO: 800) | | | | | | | |
| ACI- | 6402E10B3 | DVQLQES | SGYYWN | YISSDGS | ADNY | DVVMTQT | KSSQSLL | LVSKVDS | WQGTHFP |
| 8033- | | GPGLVKP | (SEQ ID | NNYNPSL | (SEQ ID | PLTLSVT | DSDGETY | (SEQ ID | QT |
| 6402E10- | | VQSLSLT | NO: 811) | KN | NO: 813) | IGQPASI | LN | NO: 696) | (SEQ ID |
| Ab1 | | CSVTGYS | | (SEQ ID | | SCKSSQS | (SEQ ID | | NO: 817) |
| | | ITSGYYW | | NO: 812) | | LLDSDGE | NO: 815) | | |
| | | NWIRQFP | | | | TYLNWLL | | | |
| | | GNKLEWM | | | | QRPGQSP | | | |
| | | GYISSDG | | | | KRLIYLV | | | |
| | | SNNYNPS | | | | SKVDSGV | | | |
| | | LKNRISI | | | | PDRFTGR | | | |
| | | SRDTSKN | | | | GSGTDFT | | | |
| | | QFFLKLD | | | | LKISRVE | | | |
| | | SVTTEDT | | | | AEDLGVY | | | |
| | | ATYYCVR | | | | YCWQGTH | | | |
| | | ADNYWGQ | | | | FPQTFGG | | | |
| | | GTTLTVS | | | | GTKLEIK | | | |
| | | S | | | | (SEQ ID | | | |
| | | (SEQ ID | | | | NO: 814) | | | |
| | | NO: 810) | | | | | | | |
| ACI- | 6403A4A3 | EVQLVES | TYAMN | RIRSKSN | TMDY | QIVLTQS | SASSSIS | STFNLAS | HQWSSYY |
| 8033- | | GGGLVQP | (SEQ ID | NYTTYYA | (SEQ ID | PAIMSAS | YMH | (SEQ ID | T |
| 6403A4- | | KGSLKLS | NO: 821) | DSVKD | NO: 823) | LGEEITL | (SEQ ID | NO: 826) | (SEQ ID |
| Ab1 | | CAASGFS | | (SEQ ID | | TCSASSS | NO: 825) | | NO: 827) |
| | | FNTYAMN | | NO: 822) | | ISYMHWY | | | |
| | | WVRQAPG | | | | QQKSGTS | | | |
| | | KGLEWVA | | | | PKLLIHS | | | |
| | | RIRSKSN | | | | TFNLASG | | | |
| | | NYTTYYA | | | | VPSRFSG | | | |
| | | DSVKDRF | | | | SGSGTFY | | | |
| | | TISRDDS | | | | SLTISSV | | | |
| | | ESMLYLQ | | | | EAEDAAD | | | |
| | | MNNLKTE | | | | YYCHQWS | | | |
| | | DTAMYYC | | | | SYYTFGG | | | |
| | | VRTMDYW | | | | GTKLEIK | | | |
| | | GQGTSVT | | | | (SEQ ID | | | |
| | | VSS | | | | NO: 824) | | | |
| | | (SEQ ID | | | | | | | |
| | | NO: 820) | | | | | | | |
| ACI- | 6403E11B4 | DVQLQES | SAYYWN | YISYDGS | GDVN | DVVMTQT | KSSQSLL | LVSKLDS | WQGTHFP |
| 8033- | | GPGLVKP | (SEQ ID | NNYIPSL | (SEQ ID | PLTLSVT | DGDGETY | (SEQ ID | QT |
| 6403E11- | | SQSLSLT | NO: 831) | KN | NO: 833) | IGQPASI | LN | NO: 836) | (SEQ ID |
| Ab2 | | CSVTGYS | | (SEQ ID | | SCKSSQS | (SEQ ID | | NO: 837) |
| | | ITSAYYW | | NO: 832) | | LLDGDGE | NO: 835) | | |
| | | NWIRQFP | | | | TYLNWLL | | | |
| | | GNKLEWM | | | | QRPGQSP | | | |
| | | GYISYDG | | | | KRLIYLV | | | |
| | | SNNYIPS | | | | SKLDSGV | | | |
| | | LKNRISI | | | | PDRFTGS | | | |
| | | SRDTSKN | | | | GSGTDFT | | | |
| | | QFFLKLN | | | | LKISRVE | | | |
| | | SVTTEDT | | | | AEDLGVY | | | |
| | | ATYYCIR | | | | YCWQGTH | | | |
| | | GDVNWGQ | | | | FPQTFGG | | | |
| | | GTTLTVS | | | | GTKLEIK | | | |
| | | S | | | | (SEQ ID | | | |
| | | (SEQ ID | | | | NO: 834) | | | |
| | | NO: 830) | | | | | | | |
| ACI- | 4813- | EVQLEES | SYAMH | RIRSKRS | GGREAMD | QAVVTQE | RSSTGAV | GTNNRAP | ALWYSNH |
| 7067- | R4A- | GGGLVHP | (SEQ ID | NYATYYA | F | SALTTSP | TTSNYAN | (SEQ ID | (SEQ ID |
| R4A- | G7 | KGSLKLS | NO: 841) | DSVKD | (SEQ ID | GETVTLT | (SEQ ID | NO: 846) | NO: 847) |
| G7- | | CAASGFT | | (SEQ ID | NO: 843) | CRSSTGA | NO: 845) | | |
| Ab- | | FNSYAMH | | NO: 842) | | VTTSNYA | | | |
| 1 | | WVRQAPG | | | | NWVQEKP | | | |
| (also | | KGLEWVA | | | | DHLFTGL | | | |
| termed | | RIRSKRS | | | | IGGTNNR | | | |
| ACI- | | NYATYYA | | | | APGVPAR | | | |
| 7067- | | DSVKDRF | | | | FSGSLIG | | | |
| 4813- | | TISRDDS | | | | DKAALTI | | | |
| R4A- | | QSMVYLE | | | | TGAQTED | | | |
| G7- | | MNNLEAE | | | | EAIYFCA | | | |
| rec1) | | DTATYYC | | | | LWYSNHW | | | |
| | | VRGGREA | | | | VFGGGTK | | | |
| | | MDFWGQG | | | | LTVL | | | |
| | | TSVTVSS | | | | (SEQ ID | | | |
| | | (SEQ ID | | | | NO: 844) | | | |
| | | NO: 840) | | | | | | | |

### Inhibition or Delay of Seeded Alpha-Synuclein Aggregation of Antibodies and Fab Fragments in Combination

Monoclonal anti-alpha-synuclein antibodies and Fabs were evaluated for their ability to inhibit the aggregation of alpha-synuclein in vitro. The presence of alpha-synuclein pre-formed aggregates (seeds) increases the de novo aggregation propensity of monomeric a-synuclein. Alpha-synuclein antibodies and Fabs were mixed to a final concentration of 3.28 μM or, ˜22.8 equivalents and incubated with alpha-synuclein seeds prior to adding the monomeric alpha-synuclein for the aggregation assay. For the purposes of the experiment monoclonal antibodies or fragment antibody binding (Fab) fragments were tested in this assay individually and also in combinations of two antibodies or combinations of two Fab antibody fragments. Kinetics of alpha-synuclein aggregation were monitored by thioflavin T (ThT) fluorescence. The ability of alpha-synuclein antibodies to inhibit the seeded aggregation was quantified by a percent change in the aggregation half-time, τ_{1/2}, (time to reach half-maximum ThT fluorescence signal).

Alpha-synuclein recombinant protein (rPeptide, S-1001-4) at concentration of 5 mg/mL was re-suspended and dialyzed against DPBS (Slide-A-Lyzer Mini Dialysis 10K MWCO, ThermoScientific, 88404) four times of 60 minutes each at 4° C. Higher molecular weight species were then removed by centrifugal filtration (Microcon DNA Fast Flow Centrifugal Filter Unit with Ultracel membrane, Sigma, MRCFOR100). Sonicated alpha-synuclein fibrils were diluted with PBS to a final concentration of 1.0 mg/mL. Aggregations were assembled in low-binding 96-well plates (ThermoScientific, 278752), in triplicate for each condition. Alpha-synuclein seeds were used at 1% the final concentration of monomeric alpha-synuclein (14 μM).

Alpha-synuclein seeds (34.5 pmoles) were incubated with alpha-synuclein antibodies or their corresponding Fab antibody fragments tested individually (1.64 μM or, ˜11.4 equivalents) or in combinations of two antibodies of Fabs (3.28 μM or, ˜22.8 equivalents) for 1 hour at 25° C. As a reference control, alpha-synuclein seeds were incubated without the addition of alpha-synuclein antibodies or Fabs.

Monomeric alpha-synuclein and ThT (3 mM stock solution, Sigma, D8537) were added to reach a final concentration of 14 μM and 46 μM respectively. Each aggregation was then aliquoted into 3 separate wells (65 μL/well) of the 96-well plates. Kinetic measurements were performed using an M200 Infinite Pro Microplate Reader (Tecan, Switzerland).

ThT fluorescent measurements were obtained in triplicate for each aggregation condition (technical repeats) and run twice on independent days (for a total of N=6). Aggregation half-times (τ½) were calculated from non-linear regressions using a sigmoidal dose-response (see Equation 2) (GraphPad Prism 7) and represent the time taken to reach half the maximum ThT signal.

$$\% ThT ( x ) = Bottom + \frac{( Top - Bottom )}{( 1 + {10}^{( {LogEC}_{50} - X ) - HillSlope} )} \qquad Equation 2$$

Where Bottom is a fit of the minimum ThT signal, Top is a fit of the maximum ThT signal, EC50 is the x value when the ThT signal is halfway between Bottom and Top, and the HillSlope is the steepness of the curve. Here, the aggregation half-time (τ_{1/2}) is obtained directly from EC50.

$$\% Increase {τ}_{1 / 2} = \frac{{τ}_{mAb} - {τ}_{no mAb}}{{τ}_{no mAb}} * 100 \qquad Equation 6$$

Where τ_{no mAb} is the aggregation half-time in the absence of antibody or Fab (mAb) and τ_{mAb} is the aggregation half-time in the presence of the indicated antibody or Fab.

$$Synergy = \frac{[ \% Increase {τ}_{1 / 2} ( Ab 1 + Ab 2 ) ]}{[ \% Increase ( Ab 1 ) ] + [ \% Increase ( Ab 2 ) ]} \qquad Equation 7$$

Where % Increase τ_{1/2} (Ab1+Ab2) is the percent increase in aggregation half-time in the presence of the two indicated Abs or Fabs and % Increase τ_{1/2} (Ab1) and % Increase τ_{1/2} (Ab2) are the percent increase in aggregation half-time in the presence of only the one indicated mAb or Fab.

Aggregation half-times (τ_{1/2}) were obtained using a sigmoidal fit (Equation 2). Varied time frames were used to obtain optimal fitting as ThT signals can decrease following completion of aggregation. Change in τ_{1/2} values, in the presence of the indicated antibodies, were normalized relative to the τ_{1/2} value in the absence of antibody or Fab. The percent increase in Tia values were calculated relative to the seeded aggregation in the absence of antibody or Fab (see Equation 6). A Synergy score was then calculated (see Equation 7) to assess the combinatorial effect of antibodies to inhibit the seeded aggregation. A synergy score greater than one represents a combinatorial inhibition of aggregation that is greater than the predicted inhibition from the sum of the individual antibodies or Fabs.

As shown in Table 8 significant increases in τ_{1/2} values were observed for all antibodies when tested in combination compared to when tested individually indicating a synergistic effect in delaying the seeded and/or spontaneous aggregation of alpha-synuclein. The greatest synergy is observed when combining antibodies with epitopes within the C-terminus and NAC domain or upon combining antibodies with distinct epitopes within the C-terminus. Significant increases in τ_{1/2} values were observed for all antibodies when tested in combination compared to when tested individually indicating a synergistic effect in delaying the seeded and/or spontaneous aggregation of alpha-synuclein.

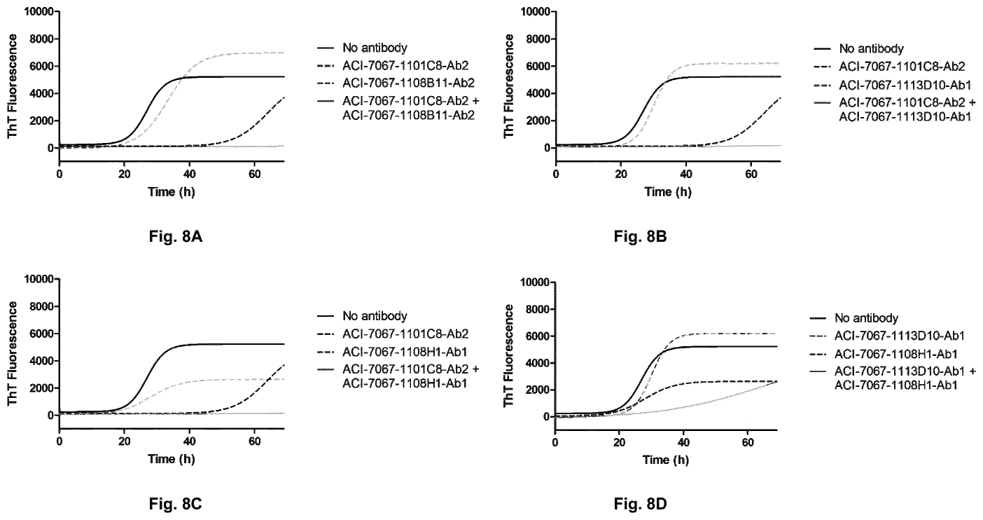

FIG. 8 shows the kinetics of alpha-synuclein aggregation in the presence of few representative antibodies tested individually or in combination of two or in the absence of antibody.

TABLE 8
Synergistic effect of monoclonal antibodies tested in combination
on aggregation half-times of seeded alpha-synuclein aggregation.

| | | Aggregation | |
|---|---|---|---|
| | | half-time | |
| Antibody | Epitope | (τ_{1/2}) (hours)¹ | Synergy² |
| No Antibody | n.a. | 26.9 | n.a. |
| ACI-7079-2506F3-Ab1 | 10-24 | 34.2 | n.a. |
| ACI-7067-1108H1-Ab1 | 65-74 | 28 | n.a. |
| ACI-7079-2503C6-Ab1 | 82-96 | 22.5 | n.a. |
| ACI-7067-1101C8-Ab2 | 124-131 | 64.0 | n.a. |
| ACI-7067-1113D10-Ab1 | 128-135 | 30.1 | n.a. |
| ACI-7067-1108B11-Ab2 | 131-140 | 33.3 | n.a. |
| ACI-7079-2506F3-Ab1 + | N-term + | 89.2 | 1.4 |
| ACI-7067-1101C8-Ab2 | C-term | | |
| ACI-7067-1108H1-Ab1 + | NAC + | 187.8 | 4.2 |
| ACI-7067-1101C8-Ab2 | C-term | | |
| ACI-7079-2503C6-Ab1 + | NAC + | 82.5 | 1.7 |
| ACI-7067-1101C8-Ab2 | C-term | | |
| ACI-7067-1113D10-Ab1 + | C-term + | 139.5 | 2.8 |
| ACI-7067-1101C8-Ab2 | C-term | | |
| ACI-7067-1108B11-Ab2 + | C-term + | 388.0 | 8.3 |
| ACI-7067-1101C8-Ab2 | C-term | | |
| ACI-7079-2506F3-Ab1 + | N-term + | 46.1 | 1.8 |
| ACI-7067-1113D10-Ab1 | C-term | | |
| ACI-7067-1108H1-Ab1 + | NAC + | 66.8 | 9.3 |
| ACI-7067-1113D10-Ab1 | C-term | | |
| ACI-7079-2503C6-Ab1 + | NAC + | 42.7 | 13.0 |
| ACI-7067-1113D10-Ab1 | C-term | | |

¹The half-time to reach the maximum ThT signal, aggregation τ_{1/2} values from in vitro alpha-synuclein aggregation in the absence of any antibody or in the presence of the indicated mAbs at 1.64 μM.
²Synergy is calculated according to equation 7.

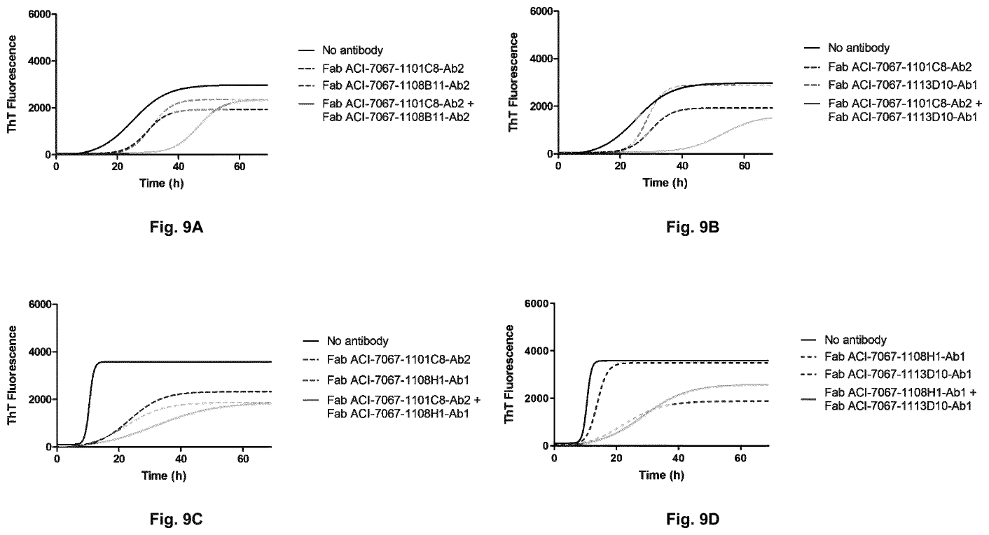

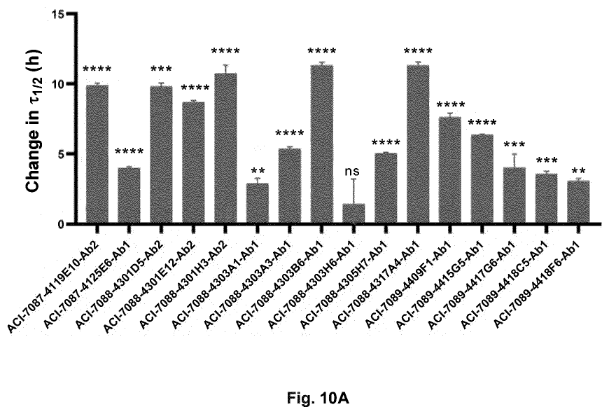

Similar results to the observed synergistic effect of monoclonal antibodies tested in combination were obtained upon testing their Fab antibody fragments in combination of two in the seeded alpha-synuclein aggregation assay (Table 9). The kinetics of alpha-synuclein aggregation in the presence of few representative Fab fragments tested individually or in combination of two or in the absence of Fab are shown in FIG. 9. As shown in Table 9 significant increases in τ_{1/2} values were observed for all Fab fragments when tested in combination compared to when tested individually indicating a synergistic effect in delaying the seeded and/or spontaneous aggregation of alpha-synuclein. The greatest synergy is observed when combining Fab antibody fragments with epitopes within the C-terminus and NAC domain or upon combining antibodies with distinct epitopes within the C-terminus.

TABLE 9
Synergistic effect of Fab antibody fragments tested in combination
on aggregation half-times of seeded alpha-synuclein aggregation.

| | | Aggregation | |
|---|---|---|---|
| | | half-time | |
| Fab antibody fragments | Epitope | (τ_{1/2}) (hours)³ | Synergy⁴ |
| No Antibody | n.a. | 18.0 | n.a. |
| Fab ACI-7067-1108H1-Ab1 | 65-74 | 19.4 | n.a. |
| Fab ACI-7067-1101C8-Ab2 | 124-131 | 26.3 | n.a. |
| Fab ACI-7067-1113D10-Ab1 | 128-135 | 21.0 | n.a. |
| Fab ACI-7067-1108B11-Ab2 | 131-140 | 22.3 | n.a. |
| Fab ACI-7067-1108H1-Ab1 + | NAC + | 27.1 | 0.8 |
| Fab ACI-7067-1101C8-Ab2 | C-term | | |
| Fab ACI-7067-1113D10-Ab1 + | C-term + | 40.5 | 2.5 |
| Fab ACI-7067-1101C8-Ab2 | C-term | | |
| Fab ACI-7067-1108B11-Ab2 + | C-term + | 37.0 | 1.6 |
| Fab ACI-7067-1101C8-Ab2 | C-term | | |
| Fab ACI-7067-1108H1-Ab1 + | NAC + | 27.5 | 1.6 |
| Fab ACI-7067-1113D10-Ab1 | C-term | | |
| Fab ACI-7067-1108H1-Ab1 + | NAC + | 22.8 | 1.3 |
| Fab ACI-7067-1108B11-Ab2 | C-term | | |

³The half-time to reach the maximum ThT signal, aggregation τ_{1/2} values from in vitro alpha-synuclein aggregation in the absence of any antibody or in the presence of the indicated mAbs at 1.64 μM.
⁴Synergy is calculated according to equation 7.

### Generation of Biparatopic Antibodies

correctly pairing the cognate heavy and light chain to produce a highly pure bispecific molecule. Heavy and light chains were synthesized (ThermoFisher scientific) and cloned into pCDNA 3.4 TOPO expression vectors (ThermoFischer scientific, A14697). CHO cells were transfected with an equimolar ratio of all 4 different chains using ExpiFectamine™ CHO transfection kit (ThermoFischer scientific, A29130) as per manufacturers recommendation. Cells were grown for 7 days at 37° C. under 120 rpm agitation. Supernatants were harvested and batch purified by protein A (Merck KGAa, GE17-5280-01). The protein resin was washed with 2×PBS and antibodies eluted with 0.1 M glycine pH 3.2. Purified antibodies were neutralized with 1/10 (VN) of 1M tris pH 7.6.

Suitable methods for fusing variable domains of heavy and light chains to engineer heavy and light chain constant domains may be performed according to Labrijn et al, PNAS, 2013 110 (13) 5145-5150 (see SEQ ID NO: 852 and SEQ ID NO: 853 for suitable constant domain sequences), Schaefer et al., PNAS, 2011, 108 (27) 11187-11192 (see SEQ ID NO: 854, SEQ ID NO: 855, SEQ ID NO: 856 for suitable constant domain sequences), WO2019/057122A1, Wu et al., Mabs, 2015 (see SEQ ID NO: 857, SEQ ID NO: 858, SEQ ID NO: 859 for suitable constant domain sequences) or Mazor et al, mAbs, 2015, 7(2): 377-389 (see SEQ ID NO: 860, SEQ ID NO: 861, SEQ ID NO: 862 for suitable constant domain sequences).

Some of the bispecific antibody generation technologies may require further antibody purification or manipulation such as partial reduction (Labrjin et al, PNAS, 2013 110 (13) 5145-5150) or standard CEX purification to remove fragment and undesired species.

Upon purification, antibodies were dialyzed with Slide-A-Lyzer™ Dialysis Cassettes (ThermoFischer scientific, 66811) into 1X PBS pH 7.4 and stored at 2-8° C.

TABLE 10
Generated alpha-synuclein biparatopic antibodies:

| Antibody name for Arm “A” | Antibody name for Arm “B” | Biparatopic Antibody code |
|---|---|---|
| ACI-7067-1108B11-Ab2 | ACI-7079-3108C10-Ab2 | ACI-1108B11_3108C10 |
| ACI-7079-3108C10-Ab2 | ACI-7079-3112H1-Ab1 | ACI-3108C10_3112H1 |
| ACI-7088-4301D5-Ab2 | ACI-7079-3108C10-Ab2 | ACI-4301D5_3108C10 |
| ACI-7079-3112H1-Ab1 | ACI-7079-3108C10-Ab2 | ACI-3112H1_3108C10 |
| ACI-7067-1108H1-Ab1 | ACI-7088-4303A3-Ab1 | ACI-1108H1_4303A3 |
| ACI-7088-4317A4-Ab1 | ACI-7067-1113D10-Ab1 | ACI-4317A4_1113D10 |
| ACI-7067-1113D10-Ab1 | ACI-7088-4317A4-Ab1 | ACI-1113D10_4317A4 |
| ACI-8033-4F3-Ab1 | ACI-8033-5A12-Ab1 | ACI-4F3_5A12 |
| ACI-7067-1101C8-Ab2 | ACI-8033-4F3-Ab1 | ACI-1101C8_4F3 |
| ACI-8033-4F3-Ab1 | ACI-7067-1101C8-Ab2 | ACI-4F3_1101C8 |
| ACI-8033-5A12-Ab1 | ACI-7079-2503C6-Ab1 | ACI-5A12_2503C6 |
| ACI-7079-2503C6-Ab1 | ACI-8033-5A12-Ab1 | ACI-2503C6_5A12 |
| ACI-8033-5A12-Ab1 | ACI-7079-3108C10-Ab2 | ACI-5A12_3108C10 |
| ACI-7079-3108C10-Ab2 | ACI-8033-5A12-Ab1 | ACI-3108C10_5A12 |
| ACI-8033-4F3-Ab1 | ACI-7088-4317A4-Ab1 | ACI-4F3_4317A4 |
| ACI-7088-4301D5-Ab2 | ACI-8033-5A12-Ab1 | ACI-4301D5_5A12 |
| ACI-7067-1108H1-Ab1 | ACI-8033-5A12-Ab1 | ACI-1108H1_5A12 |
| ACI-7079-3112H1-Ab1 | ACI-8033-5A12-Ab1 | ACI-3112H1_5A12 |
| ACI-7067-1101C8-Ab2 | ACI-8033-5A12-Ab1 | ACI-1101C8_5A12 |
| ACI-7079-2503C6-Ab1 | ACI-7067-1101C8-Ab2 | ACI-2503C6_1101C8 |
| ACI-8033-27D8-Ab1 | ACI-7067-1101C8-Ab2 | ACI-27D8_1101C8 |
| ACI-8033-27D8-Ab1 | ACI-7088-4317A4-Ab1 | ACI-27D8_4317A4 |
| ACI-8033-27D8-Ab1 | ACI-7088-4301D5-Ab2 | ACI-27D8_4301D5 |
| ACI-8033-27D8-Ab1 | ACI-8033-1F8-Ab1 | ACI-27D8_1F8 |
| ACI-8033-27D8-Ab1 | ACI-7088-4301H3-Ab2 | ACI-27D8_4301H3 |
| ACI-7067-1108B11-Ab2 | ACI-8033-27D8-Ab1 | ACI-1108B11_27D8 |
| ACI-7067-1108B11-Ab2 | ACI-8033-7A2-Ab1 | ACI-1108B11_7A2 |
| ACI-7088-4301H3-Ab2 | ACI-7079-3108C10-Ab2 | ACI-4301H3_3108C10 |
| ACI-7067-1101C8-Ab2 | ACI-7088-4317A4-Ab1 | ACI-1101C8_4317A4 |
| ACI-7079-3108C10-Ab2 | ACI-7067-1101C8-Ab2 | ACI-3108C10_1101C8 |
| ACI-7067-1101C8-Ab2 | ACI-7088-4301E12-Ab2 | ACI-1101C8_4301E12 |
| ACI-7079-3112H1-Ab1 | ACI-7067-1101C8-Ab2 | ACI-3112H1_1101C8 |
| ACI-7067-1101C8-Ab2 | ACI-7079-3101E3-Ab1 | ACI-1101C8_3101E3 |

### Affinity Measurements of Alpha-Synuclein Biparatopic Antibodies on Alpha-Synuclein Monomers and Alpha-Synuclein Fibrils by SPR

Affinity measurements were performed on an surface plasmon resonance (SPR) instrument (Biacore 8K, GE Healthcare Life Sciences) using CM5 Series S sensor chips (GE Healthcare, BR-1005-30). Channels 1-8 were activated with a fresh solution of EDC/NHS (Amine Coupling Kit, 1:1 ratio of both reagents, GE Healthcare, BR-1006-33). The goat anti-human antibody (Jackson Immunology, 109-005-098) was captured at a concentration of 30 μg/mL diluted in 10 mM sodium acetate (pH 5.0). Following, all unreacted activated ester groups were capped with 1 M ethanolamine (GE Healthcare, BR-1006-33). Any non-covalently bound antibodies were removed by three successive regenerations of 10 mM Glycine pH 1.7 (GE Healthcare, 28-9950-84). Immobilization levels were evaluated following ethanolamine capping (Bound) and finally following regeneration (Final). Non-covalent immobilization of alpha-synuclein biparatopic antibodies on flow cell 2 of channels 1-8 was performed at a final concentration of 5 μg/mL, diluted in 10 mM sodium acetate pH 5.5 (GE Healthcare, BR-1003-52). Non-covalent immobilization of an isotype control antibody on flow cell 1 of channels 1-8 was performed at a final concentration of 5 μg/mL, diluted in 10 mM sodium acetate pH 5.5 (GE Healthcare, BR-1003-52).

Binding affinity of alpha-synuclein biparatopic antibodies to monomeric or fibrillar alpha-synuclein species was performed using a single-cycle kinetics method. The instrument was primed with 1×HBS-P+ buffer (10× stock from GE Healthcare, BR-1003-52 diluted in Milli-Q water). Injections of monomeric alpha-synuclein (Boston Biochem, SP-485), increasing in concentration from 0.62-50 nM prepared from serial 2-fold dilutions, were performed with contact times of 300 sec/injection at a flow rate of 30 μL/min. A dissociation phase of 900 sec followed the final 50 nM injection. Regeneration of the sensor to the goat anti-human antibody layer was achieved using 3 regenerations of 10 mM Glycine pH 1.7. Injections of alpha-synuclein fibrils of increasing in concentration from 5.56-450 nM prepared from serial 2-fold dilutions, were performed with contact times of 300 sec/injection at a flow rate of 30 μL/min. A dissociation phase of 900 sec followed the final 450 nM injection. Regeneration of the sensor to the goat anti-mouse antibody layer was achieved using 3 regenerations of 10 mM Glycine pH 1.7. Results obtained from single-cycle kinetics were evaluated by Biacore 8K evaluation software with 1:1 binding homogenous Langmuir model with a preceeding buffer injection as a blank subtraction. The following kinetic parameters were obtained: on-rate (ka), off-rate (kd), affinity constant (KD, ratio of kd by ka), maximum response (Rmax), and goodness of fit (Chi2).

Kinetic constants were determined from 1:1 homogenous binding models for all cases. The kinetic fitting parameters from single-cycle kinetics affinity measurements by SPR are shown in Table 11. The majority of the biparatopic antibodies, such as ACI-4F3_4317A4, ACI-2503C6_1101 C8 ACI-3108C10_3112H1, ACI-3112H1_3108C10, and ACI-1101C8_4301E12 demonstrate a binding preference for fibrillar alpha-synuclein. Moreover, some biparatopic antibodies, such as, ACI-5A12_3108C10, ACI-4F3_4317A4, ACI-2503C6_1101C8 0 and ACI-1113D104317A4 display at least 100-fold slower dissociation rates (Kd) from fibrillar alpha-synuclein compared to monomeric alpha-synuclein.

TABLE 11
Affinity measurements obtained by SPR

| | Alpha-synuclein monomers | | | Alpha-synuclein fibrils | | |
|---|---|---|---|---|---|---|
| Biparatopic antibody | ka (1/Ms) | kd (1/s) | KD (nM) | ka (1/Ms) | kd (1/s) | KD (nM) |
| ACI-3108C10_3112H1 | 2.28E+03 | 2.30E−02 | 1.01E−05 | 1.35E+04 | 3.15E−04 | 2.34E−08 |
| ACI-3112H1_3108C10 | 5.54E+03 | 1.62E−02 | 2.92E−06 | 7.65E+03 | 4.72E−04 | 6.17E−08 |
| ACI-1108H1_4303A3 | 1.43E+06 | 1.29E−03 | 9.03E−10 | 7.36E+04 | 1.49E−05 | 2.03E−10 |
| ACI-4317A4_1113D10 | 1.39E+06 | 6.75E−04 | 4.86E−10 | 4.92E+04 | 7.77E−06 | 1.58E−10 |
| ACI-1113D10_4317A4 | 1.23E+06 | 5.14E−04 | 4.16E−10 | 5.68E+04 | 3.48E−07 | 6.14E−12 |
| ACI-5A12_3108C10 | 1.69E+09 | 5.83E+01 | 3.45E−08 | 1.12E+04 | 9.19E−05 | 8.21E−09 |
| ACI-3108C10_5A12 | 9.13E+08 | 8.05E+00 | 8.82E−09 | 1.05E+04 | 1.13E−04 | 1.08E−08 |
| ACI-4F3_4317A4 | 8.84E+05 | 1.03E−03 | 1.17E−09 | 5.17E+04 | 7.84E−08 | 1.52E−12 |
| ACI-1101C8_5A12 | 1.10E+06 | 4.20E−03 | 3.82E−09 | 4.67E+04 | 6.13E−05 | 1.31E−09 |
| ACI-2503C6_1101C8 | 2.20E+05 | 2.50E−04 | 1.14E−09 | 6.16E+04 | 1.53E−07 | 2.48E−12 |
| ACI-27D8_4317A4 | 7.94E+06 | 2.83E−02 | 3.57E−09 | 6.54E+04 | 5.80E−05 | 8.87E−10 |
| ACI-27D8_4301D5 | 1.37E+06 | 1.47E−01 | 1.07E−07 | 1.31E+04 | 2.19E−04 | 1.67E−08 |
| ACI-4301H3_3108C10 | 1.04E+06 | 3.61E−02 | 3.48E−08 | 8.22E+03 | 4.18E−04 | 5.08E−08 |
| ACI-1101C8_4317A4 | 2.35E+06 | 1.58E−03 | 6.71E−10 | 6.93E+04 | 3.00E−05 | 4.33E−10 |
| ACI-1101C8_4301E12 | 2.24E+05 | 4.44E−04 | 1.98E−09 | 4.49E+04 | 9.65E−07 | 2.15E−11 |

### Inhibition or Delay of Seeded Alpha-Synuclein Aggregation by Alpha-Synuclein Biparatopic Antibodies

Biparatopic anti-alpha-synuclein antibodies were evaluated for their ability to inhibit the aggregation of alpha-synuclein in vitro. The presence of alpha-synuclein pre-formed aggregates (seeds) increases the de novo aggregation propensity of monomeric a-synuclein. Alpha-synuclein biparatopic antibodies were incubated with alpha-synuclein seeds prior to adding the monomeric alpha-synuclein for the aggregation assay. Kinetics of alpha-synuclein aggregation were monitored by thioflavin T (ThT) fluorescence. The ability of alpha-synuclein biparatopic antibodies to inhibit the seeded aggregation was quantified by a percent change in the aggregation half-time (time to reach half-maximum ThT fluorescence signal).

Alpha-synuclein recombinant protein (rPeptide, S-1001-4) at concentration of 5 mg/mL was re-suspended and dialyzed against DPBS (Slide-A-Lyzer Mini Dialysis 10K MWCO, ThermoScientific, 88404) four times of 60 minutes each at 4° C. Higher molecular weight species were then removed by centrifugal filtration (Microcon DNA Fast Flow Centrifugal Filter Unit with Ultracel membrane, Sigma, MRCFOR100). Sonicated alpha-synuclein fibrils were diluted with PBS to a final concentration of 1.0 mg/mL. Aggregations were assembled in a low-binding 96-well plates (ThermoScientific, 278752), in triplicate for each condition. Alpha-synuclein seeds were used at 1% the final concentration of monomeric alpha-synuclein (14 μM).

Alpha-synuclein seeds (34.5 pmoles) were incubated with alpha-synuclein biparatopic antibodies (787 pmoles, ˜22.8 equivalents) for 1 hour at at 25° C. As a reference control, alpha-synuclein seeds were incubated without the addition of alpha-synuclein biparatopic antibodies. To control for any non-alpha-synuclein specific effect from the antibodies, the mouse IgG2a isotype control (IgG2a) (ThermoFisher, 02-6200) was used as a negative control.

Monomeric alpha-synuclein and ThT (3 mM stock solution, Sigma, D8537) were added to reach a final concentration of 14 μM and 46 μM respectively. Each aggregation was then aliquoted into 3 separate wells (65 μL/well) of the 96-well plates. Kinetic measurements were performed using an M200 Infinite Pro Microplate Reader (Tecan, Switzerland).

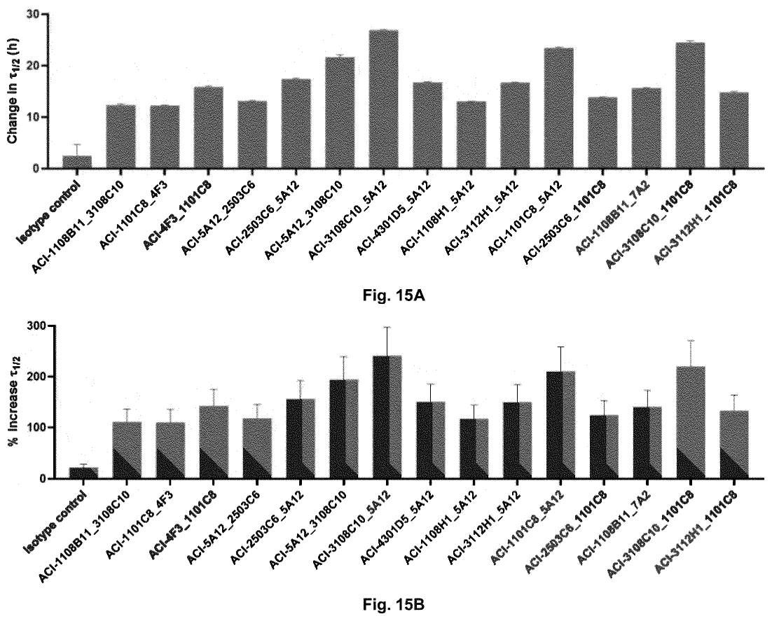

ThT fluorescent measurements were obtained in triplicate for each aggregation condition (technical repeats). Aggregation half-times (τ½) were calculated from non-linear regressions using a sigmoidal dose-response (see Equation 2) (GraphPad Prism 7) and represent the time taken to reach half the maximum ThT signal. Varied time frames were used to obtain optimal fitting as ThT signals can decrease following completion of aggregation. Change in τ_{1Q} values, in the presence of the indicated antibodies, were normalized relative to the τ_{1/2} value in the absence of antibody. FIG. 15A shows the comparison of changes in τ_{1/2} values as normalized to the aggregation in the absence of antibody. The percent increase in τ_{1/2} values were calculated relative to the seeded aggregation in the absence of antibody (see Equation 4). FIG. 15B shows the calculated percent increase in τ_{1/2} values upon pre-incubation of alpha-synuclein seeds with the indicated antibodies proving the good efficacy of biparatopic antibodies in delaying the seeded and/or spontaneous aggregation of alpha-synuclein. ACI-3108C10_5A12, ACI-3108C10_1101C8, and ACI-1101C8_5A12 demonstrated the largest increase in Tia values, closely followed by ACI-5A12_3108C10, ACI-2503C6_5A12, ACI-4301D5_5A12, and ACI-3112H1_5A12.

### Inhibiting Alpha-Synuclein Propagation in Cells

Biparatopic anti-alpha-synuclein antibodies were evaluated for their ability to inhibit alpha-synuclein aggregation in a cellular model. The addition of alpha-synuclein seeds to the cells triggers the aggregation of endogenous monomeric alpha-synuclein, resulting in the formation of de novo aggregates. Antibodies binding to pathological conformations of alpha-synuclein would lead to the depletion of seeding-competent alpha-synuclein species and consequently reduced number of de novo aggregates This cellular model was used to assess the impact of anti-alpha-synuclein biparatopic antibodies on the seeding capacity and aggregation of alpha-synuclein. Biparatopic anti-alpha-synuclein antibodies were co-incubated in vitro with a-syn seeds to immunodeplete the pathological alpha-synuclein conformations, the remaining material was added onto the cells. The ability of anti-alpha-synuclein biparatopic antibodies to inhibit seeded aggregation was quantified as a percent change in the number of alpha-synuclein aggregates observed.

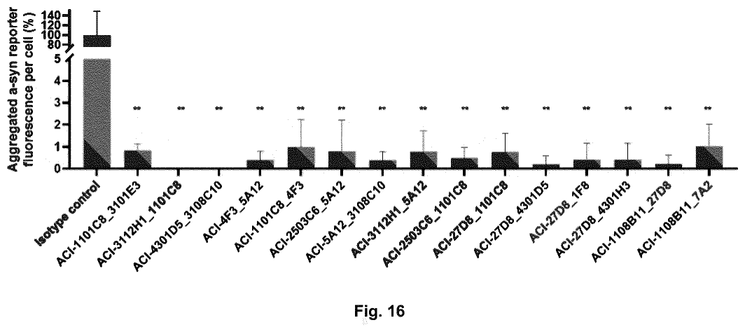

In this cellular assay, single concentration screening of alpha-synuclein biparatopic antibodies or an isotype control antibody was performed. Immunodepletion of alpha-synuclein seeds, or isotype control antibody, was performed using Dynabeads™ Protein G Immunoprecipitation Kit (Thermoscientific, 10007D). Briefly, 0.4 mg of Dynabeads™ were loaded with 2.8 pg of antibody according to the manufacturer's protocol. Alpha-synuclein seeds (0.05 pg/well) were incubated with 0.5 molar equivalents of antibody loaded Dynabeads™, resuspended in Opti-MEM™ (Life Technologies, 31985070), at room temperature under constant rotation and shaking for 30 minutes. The alpha-synuclein immunodepleted fraction was collected and then incubated with 200 ng/well Lipofectamine™ 2000 Transfection Reagent (Life Technologies, 11668019) for 20 minutes at room temperature. The alpha-synuclein seed immunodepleted fraction/lipofectamine mixture was then added to cells plated 24 hours before treatment at a density of 8000 cells/well. Cells were placed back in the incubator (at 37° C. with 5% C02). At 42 hours, post transduction, cells were fixed with an equal volume of cold 2% Triton X-100, 8% PFA in PBS, and Hoechst 33342 (1:10,000). Medium was removed and washed three times with PBS, fixed cells were left in PBS, kept protected from light, and high-content imaging analysis was performed to detect and quantify the de novo alpha-synuclein aggregates. Use of an intrinsically fluorescent reporter protein allowed for the detection of de novo alpha-synuclein aggregates. The percent aggregates formed were then calculated relative to the isotype control condition. FIG. 16 shows the percent aggregates formed. All of the antibodies tested demonstrated the capacity to reduce significantly the de novo aggregates formation relative to the isotype control. ACI-3112H1_1101C8, ACI-4301D5_3108C10, ACI-27D8_4301D5, ACI-1108B11_27D8 and ACI-4F3_5A12 demonstrated the greatest reduction in de novo aggregate formation.

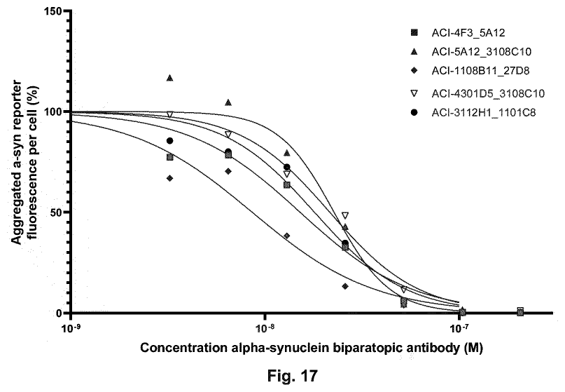

For determination of dose response curves, the molar equivalents of alpha-synuclein biparatopic antibodies were varied from 1.0 to 0.016, using serial 2-fold dilutions, relative to the concentration of alpha-synuclein seeds. The percent aggregates formed were then calculated relative to conditions in the absence of antibodies. IC50 values were obtained from fitting using Equation 8 (GraphPad Prism 7). FIG. 17 shows the plotted dose-response curved and calculated IC50 values. FIG. 17 shows that biparatopic antibodies ACI-3112H1_1101C8, ACI-4301D5_3108C10, ACI-1108B11_27D8, ACI-5A12_3108C10 and ACI-4F3_5A12 have the capacity to reduce the alpha-synuclein seeding capacity in a dose-dependent manner.

$$Y = \frac{Bottom + ( Top - Bottom )}{1 + {10}^{( log ! {C}_{50} - X ) * Hill Slope}} \qquad Equation 8$$

Based on meta-analysis of the various experiments performed, all antibodies were highly effective. According to the data set shown in the examples, the following biparatopic antibodies were considered the best performing among the group: ACI-4301D5_3108C10, ACI-5A12_3108C10, ACI-3108C10_5A12, ACI-4F3_4317A4, ACI-1101C8_5A12, ACI-2503C6_1101C8, ACI-27D8_4301D5, ACI-3108C10_1101C8 and ACI-3112H1_1101C8. Within this group, with ACI-5A12_3108C10 and ACI-2503C6_1101C8 being the best performing antibodies.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

1.-64. (canceled)

65. An alpha-synuclein biparatopic antibody or functional fragment thereof, or a mixture comprising at least two monospecific antibodies or functional fragments thereof, which binds at least two distinct epitopes of alpha-synuclein, preferably human alpha-synuclein of SEQ ID NO: 1.

66. The alpha-synuclein biparatopic antibody or functional fragment thereof, or a mixture comprising at least two monospecific antibodies or functional fragments thereof, according to claim 65, which inhibits and/or delays seeded and/or spontaneous alpha-synuclein aggregation.

67. An alpha-synuclein biparatopic antibody or functional fragment thereof, or a mixture comprising at least two monospecific antibodies or functional fragments thereof, according to claim 65, comprising a first binding site which binds a first epitope within amino acid residues 96-140 of human alpha-synuclein of SEQ ID NO: 1, and a second binding site which binds to a second distinct epitope within human alpha-synuclein of SEQ ID NO: 1; optionally wherein the second distinct epitope is situated within amino acids residues 60-95 or 96-140.

68. The alpha-synuclein biparatopic antibody or functional fragment thereof, or a mixture comprising at least two monospecific antibodies or functional fragments thereof, according to claim 65, comprising a first binding site which binds a first epitope situated within amino acid residues 1-15 (SEQ ID NO: 121), 10-24 (SEQ ID NO: 122), 15-45 (SEQ ID NO: 138), 19-33 (SEQ ID NO: 123), 28-50 (SEQ ID NO: 139), 28-42 (SEQ ID NO: 124), 31-60 (SEQ ID NO: 146), 36-40 (SEQ ID NO: 2), 37-51 (SEQ ID NO: 125), 51-57 (SEQ ID NO: 3), 51-58 (SEQ ID NO: 136), 65-74 (SEQ ID NO: 4), 65-81 (SEQ ID NO: 5), 81-120 (SEQ ID NO: 137), 82-96 (SEQ ID NO: 130), 91-105 (SEQ ID NO: 131), 93-95 (GFV), 96-140 (SEQ ID NO: 147), 100-114 (SEQ ID NO: 132), 109-123 (SEQ ID NO: 133), 118-132 (SEQ ID NO: 134), 124-131 (SEQ ID NO: 7), 127-140 (SEQ ID NO: 135), 128-135 (SEQ ID NO: 8) or 131-140 (SEQ ID NO: 9) of human alpha-synuclein of SEQ ID NO: 1, and a second binding site which binds to a second distinct epitope within human alpha-synuclein of SEQ ID NO: 1; optionally wherein the second distinct epitope is situated within amino acid residues 1-15 (SEQ ID NO: 121), 10-24 (SEQ ID NO: 122), 15-45 (SEQ ID NO: 138), 19-33 (SEQ ID NO: 123), 28-50 (SEQ ID NO: 139), 28-42 (SEQ ID NO: 124), 31-60 (SEQ ID NO: 146), 36-40 (SEQ ID NO: 2), 37-51 (SEQ ID NO: 125), 51-57 (SEQ ID NO: 3), 51-58 (SEQ ID NO: 136), 65-74 (SEQ ID NO: 4), 65-81 (SEQ ID NO: 5), 81-120 (SEQ ID NO: 137), 82-96 (SEQ ID NO: 130), 91-105 (SEQ ID NO: 131), 93-95 (GFV), 96-140 (SEQ ID NO: 147), 100-114 (SEQ ID NO: 132), 109-123 (SEQ ID NO: 133), 118-132 (SEQ ID NO: 134), 124-131 (SEQ ID NO: 7), 127-140 (SEQ ID NO: 135), 128-135 (SEQ ID NO: 8) or 131-140 (SEQ ID NO: 9) of human alpha-synuclein of SEQ ID NO: 1.

69. The alpha-synuclein biparatopic antibody or functional fragment thereof, or a mixture comprising at least two monospecific antibodies or functional fragments thereof, according to claim 65, comprising a first binding site which binds a first epitope and a second distinct binding site which binds to a second distinct epitope, wherein:
a. the first epitope is situated within amino acid residues 65-74 (SEQ ID NO: 4) of human alpha-synuclein of SEQ ID NO: 1 and the second epitope is situated within amino acid residues 124-131 (SEQ ID NO: 7) of human alpha-synuclein of SEQ ID NO: 1; or
b. the first epitope is situated within amino acid residues 128-135 (SEQ ID NO: 8) of human alpha-synuclein of SEQ ID NO: 1 and the second epitope is situated within amino acid residues 124-131 (SEQ ID NO: 7) of human alpha-synuclein of SEQ ID NO: 1; or
c. the first epitope is situated within amino acid residues 131-140 (SEQ ID NO: 9) of human alpha-synuclein of SEQ ID NO: 1 and the second epitope is situated within amino acid residues 124-131 (SEQ ID NO: 7) of human alpha-synuclein of SEQ ID NO: 1; or
d. the first epitope is situated within amino acid residues 65-74 (SEQ ID NO: 4) of human alpha-synuclein of SEQ ID NO: 1 and the second epitope is situated within amino acid residues 128-135 (SEQ ID NO: 8) of human alpha-synuclein of SEQ ID NO: 1; or
e. the first epitope is situated within amino acid residues 65-74 (SEQ ID NO: 4) of human alpha-synuclein of SEQ ID NO: 1 and the second epitope is situated within amino acid residues 131-140 (SEQ ID NO: 9) of human alpha-synuclein of SEQ ID NO: 1; or
f. the first epitope is situated within amino acid residues 10-24 (SEQ ID NO: 122) of human alpha-synuclein of SEQ ID NO: 1 and the second epitope is situated within amino acid residues 124-131 (SEQ ID NO: 7) of human alpha-synuclein of SEQ ID NO: 1; or
g. the first epitope is situated within amino acid residues 82-96 (SEQ ID NO: 130) of human alpha-synuclein of SEQ ID NO: 1 and the second epitope is situated within amino acid residues 124-131 (SEQ ID NO: 7) of human alpha-synuclein of SEQ ID NO: 1; or
h. the first epitope is situated within amino acid residues 10-24 (SEQ ID NO: 122) of human alpha-synuclein of SEQ ID NO: 1 and the second epitope is situated within amino acid residues 128-135 (SEQ ID NO: 8) of human alpha-synuclein of SEQ ID NO: 1; or
i. the first epitope is situated within amino acid residues 82-96 (SEQ ID NO: 130) of human alpha-synuclein of SEQ ID NO: 1 and the second epitope is situated within amino acid residues 128-135 (SEQ ID NO: 8) of human alpha-synuclein of SEQ ID NO: 1; or
j. the first epitope is situated within amino acid residues 131-140 (SEQ ID NO: 9) of human alpha-synuclein of SEQ ID NO: 1 and the second epitope is situated within amino acid residues 28-50 (SEQ ID NO: 139) of human alpha-synuclein of SEQ ID NO: 1; or
k. the first epitope is situated within amino acid residues 131-140 (SEQ ID NO: 9) of human alpha-synuclein of SEQ ID NO: 1 and the second epitope is situated within amino acid residues 100-114 (SEQ ID NO: 132) of human alpha-synuclein of SEQ ID NO: 1; or
l. the first epitope is situated within amino acid residues 28-50 (SEQ ID NO: 139) of human alpha-synuclein of SEQ ID NO: 1 and the second epitope is situated within amino acid residues 91-105 (SEQ ID NO: 131) of human alpha-synuclein of SEQ ID NO: 1; or
m. the first epitope is situated within amino acid residues 28-42 (SEQ ID NO: 124) of human alpha-synuclein of SEQ ID NO: 1 and the second epitope is situated within amino acid residues 28-50 (SEQ ID NO: 139) of human alpha-synuclein of SEQ ID NO: 1; or
n. the first epitope is situated within amino acid residues 37-51 (SEQ ID NO: 125) of human alpha-synuclein of SEQ ID NO: 1 and the second epitope is situated within amino acid residues 28-50 (SEQ ID NO: 139) of human alpha-synuclein of SEQ ID NO: 1; or
o. the first epitope is situated within amino acid residues 28-42 (SEQ ID NO: 124) and 37-51 (SEQ ID NO: 125) of human alpha-synuclein of SEQ ID NO: 1 and the second epitope is situated within amino acid residues 28-50 (SEQ ID NO: 139) of human alpha-synuclein of SEQ ID NO: 1; or
p. the first epitope is situated within amino acid residues 65-74 (SEQ ID NO: 4) of human alpha-synuclein of SEQ ID NO: 1 and the second epitope is situated within amino acid residues 37-51 (SEQ ID NO: 125) of human alpha-synuclein of SEQ ID NO: 1; or
q. the first epitope is situated within amino acid residues 1-15 (SEQ ID NO: 121) of human alpha-synuclein of SEQ ID NO: 1 and the second epitope is situated within amino acid residues 128-135 (SEQ ID NO: 8) of human alpha-synuclein of SEQ ID NO: 1; or
r. the first epitope is situated within amino acid residues 91-105 (SEQ ID NO: 131) of human alpha-synuclein of SEQ ID NO: 1 and the second epitope is situated within amino acid residues 100-114 (SEQ ID NO: 132) of human alpha-synuclein of SEQ ID NO: 1; or
s. the first epitope is situated within amino acid residues 91-105 (SEQ ID NO: 131) of human alpha-synuclein of SEQ ID NO: 1 and the second epitope is situated within amino acid residues 109-123 (SEQ ID NO: 133) of human alpha-synuclein of SEQ ID NO: 1; or
t. the first epitope is situated within amino acid residues 91-105 (SEQ ID NO: 131) of human alpha-synuclein of SEQ ID NO: 1 and the second epitope is situated within amino acid residues 100-114 (SEQ ID NO: 132) and 109-123 (SEQ ID NO: 133) of human alpha-synuclein of SEQ ID NO: 1
u. the first epitope is situated within amino acid residues 124-131 (SEQ ID NO: 7) of human alpha-synuclein of SEQ ID NO: 1 and the second epitope is situated within amino acid residues 91-105 (SEQ ID NO: 131) of human alpha-synuclein of SEQ ID NO: 1; or
v. the first epitope is situated within amino acid residues 100-114 (SEQ ID NO: 132) of human alpha-synuclein of SEQ ID NO: 1 and the second epitope is situated within amino acid residues 82-96 (SEQ ID NO: 130) of human alpha-synuclein of SEQ ID NO: 1; or
w. the first epitope is situated within amino acid residues 109-123 (SEQ ID NO: 133) of human alpha-synuclein of SEQ ID NO: 1 and the second epitope is situated within amino acid residues 82-96 (SEQ ID NO: 130) of human alpha-synuclein of SEQ ID NO: 1; or
x. the first epitope is situated within amino acid residues 100-114 (SEQ ID NO: 132) and 109-123 (SEQ ID NO: 133) of human alpha-synuclein of SEQ ID NO: 1 and the second epitope is situated within amino acid residues 82-96 (SEQ ID NO: 130) of human alpha-synuclein of SEQ ID NO: 1; or
y. the first epitope is situated within amino acid residues 100-114 (SEQ ID NO: 132) of human alpha-synuclein of SEQ ID NO: 1 and the second epitope is situated within amino acid residues 28-50 (SEQ ID NO: 139) of human alpha-synuclein of SEQ ID NO: 1; or
z. the first epitope is situated within amino acid residues 109-123 (SEQ ID NO: 133) of human alpha-synuclein of SEQ ID NO: 1 and the second epitope is situated within amino acid residues 28-50 (SEQ ID NO: 139) of human alpha-synuclein of SEQ ID NO: 1; or
aa. the first epitope is situated within amino acid residues 100-114 (SEQ ID NO: 132) and 109-123 (SEQ ID NO: 133) of human alpha-synuclein of SEQ ID NO: 1 and the second epitope is situated within amino acid residues 28-50 (SEQ ID NO: 139) of human alpha-synuclein of SEQ ID NO: 1; or
bb. the first epitope is situated within amino acid residues 109-123 (SEQ ID NO: 133) of human alpha-synuclein of SEQ ID NO: 1 and the second epitope is situated within amino acid residues 100-114 (SEQ ID NO: 132) of human alpha-synuclein of SEQ ID NO: 1; or
cc. the first epitope is situated within amino acid residues 100-114 (SEQ ID NO: 132) of human alpha-synuclein of SEQ ID NO: 1 and the second epitope is situated within amino acid residues 100-114 (SEQ ID NO: 132) of human alpha-synuclein of SEQ ID NO: 1; or
dd. the first epitope is situated within amino acid residues 100-114 (SEQ ID NO: 132) and 109-123 (SEQ ID NO: 133) of human alpha-synuclein of SEQ ID NO: 1 and the second epitope is situated within amino acid residues 100-114 (SEQ ID NO: 132) of human alpha-synuclein of SEQ ID NO: 1; or
ee. the first epitope is situated within amino acid residues 91-105 (SEQ ID NO: 131) of human alpha-synuclein of SEQ ID NO: 1 and the second epitope is situated within amino acid residues 1-15 (SEQ ID NO: 121) of human alpha-synuclein of SEQ ID NO: 1; or
ff. the first epitope is situated within amino acid residues 28-42 (SEQ ID NO: 124) of human alpha-synuclein of SEQ ID NO: 1 and the second epitope is situated within amino acid residues 100-114 (SEQ ID NO: 132) of human alpha-synuclein of SEQ ID NO: 1; or
gg. the first epitope is situated within amino acid residues 28-42 (SEQ ID NO: 124) of human alpha-synuclein of SEQ ID NO: 1 and the second epitope is situated within amino acid residues 109-123 (SEQ ID NO: 133) of human alpha-synuclein of SEQ ID NO: 1; or
hh. the first epitope is situated within amino acid residues 37-51 (SEQ ID NO: 125) of human alpha-synuclein of SEQ ID NO: 1 and the second epitope is situated within amino acid residues 100-114 (SEQ ID NO: 132) of human alpha-synuclein of SEQ ID NO: 1; or
ii. the first epitope is situated within amino acid residues 37-51 (SEQ ID NO: 125) of human alpha-synuclein of SEQ ID NO: 1 and the second epitope is situated within amino acid residues 109-123 (SEQ ID NO: 133) of human alpha-synuclein of SEQ ID NO: 1; or
jj. the first epitope is situated within amino acid residues 28-42 (SEQ ID NO: 124) and 37-51 (SEQ ID NO: 125) of human alpha-synuclein of SEQ ID NO: 1 and the second epitope is situated within amino acid residues 100-114 (SEQ ID NO: 132) and 109-123 (SEQ ID NO: 133) of human alpha-synuclein of SEQ ID NO: 1; or
kk. the first epitope is situated within amino acid residues 65-74 (SEQ ID NO: 4) of human alpha-synuclein of SEQ ID NO: 1 and the second epitope is situated within amino acid residues 100-114 (SEQ ID NO: 132) of human alpha-synuclein of SEQ ID NO: 1; or
ll. the first epitope is situated within amino acid residues 65-74 (SEQ ID NO: 4) of human alpha-synuclein of SEQ ID NO: 1 and the second epitope is situated within amino acid residues 109-123 (SEQ ID NO: 133) of human alpha-synuclein of SEQ ID NO: 1; or
mm. the first epitope is situated within amino acid residues 65-74 (SEQ ID NO: 4) of human alpha-synuclein of SEQ ID NO: 1 and the second epitope is situated within amino acid residues 100-114 (SEQ ID NO: 132) and 109-123 (SEQ ID NO: 133) of human alpha-synuclein of SEQ ID NO: 1; or
nn. the first epitope is situated within amino acid residues 124-131 (SEQ ID NO: 7) of human alpha-synuclein of SEQ ID NO: 1 and the second epitope is situated within amino acid residues 100-114 (SEQ ID NO: 132) of human alpha-synuclein of SEQ ID NO: 1; or
oo. the first epitope is situated within amino acid residues 124-131 (SEQ ID NO: 7) of human alpha-synuclein of SEQ ID NO: 1 and the second epitope is situated within amino acid residues 109-123 (SEQ ID NO: 133) of human alpha-synuclein of SEQ ID NO: 1; or
pp. the first epitope is situated within amino acid residues 124-131 (SEQ ID NO: 7) of human alpha-synuclein of SEQ ID NO: 1 and the second epitope is situated within amino acid residues 100-114 (SEQ ID NO: 132) and 109-123 (SEQ ID NO: 133) of human alpha-synuclein of SEQ ID NO: 1; or
qq. the first epitope is situated within amino acid residues 81-120 (SEQ ID NO: 137) of human alpha-synuclein of SEQ ID NO: 1 and the second epitope is situated within amino acid residues 124-131 (SEQ ID NO: 7) of human alpha-synuclein of SEQ ID NO: 1; or
rr. the first epitope is situated within amino acid residues 81-120 (SEQ ID NO: 137) of human alpha-synuclein of SEQ ID NO: 1 and the second epitope is situated within amino acid residues 1-15 (SEQ ID NO: 121) of human alpha-synuclein of SEQ ID NO: 1; or
ss. the first epitope is situated within amino acid residues 81-120 (SEQ ID NO: 137) of human alpha-synuclein of SEQ ID NO: 1 and the second epitope is situated within amino acid residues 28-42 (SEQ ID NO: 124) of human alpha-synuclein of SEQ ID NO: 1; or
tt. the first epitope is situated within amino acid residues 81-120 (SEQ ID NO: 137) of human alpha-synuclein of SEQ ID NO: 1 and the second epitope is situated within amino acid residues 37-51 (SEQ ID NO: 125) of human alpha-synuclein of SEQ ID NO: 1; or
uu. the first epitope is situated within amino acid residues 81-120 (SEQ ID NO: 137) of human alpha-synuclein of SEQ ID NO: 1 and the second epitope is situated within amino acid residues 28-42 (SEQ ID NO: 124) and 37-51 (SEQ ID NO: 125) of human alpha-synuclein of SEQ ID NO: 1; or
w. the first epitope is situated within amino acid residues 81-120 (SEQ ID NO: 137) of human alpha-synuclein of SEQ ID NO: 1 and the second epitope is situated within amino acid residues 82-96 (SEQ ID NO: 130) of human alpha-synuclein of SEQ ID NO: 1; or
ww. the first epitope is situated within amino acid residues 81-120 (SEQ ID NO: 137) of human alpha-synuclein of SEQ ID NO: 1 and the second epitope is situated within amino acid residues 91-105 (SEQ ID NO: 131) of human alpha-synuclein of SEQ ID NO: 1; or
xx. the first epitope is situated within amino acid residues 131-140 (SEQ ID NO: 9) of human alpha-synuclein of SEQ ID NO: 1 and the second epitope is situated within amino acid residues 81-120 (SEQ ID NO: 137) of human alpha-synuclein of SEQ ID NO: 1; or
yy. the first epitope is situated within amino acid residues 131-140 (SEQ ID NO: 9) of human alpha-synuclein of SEQ ID NO: 1 and the second epitope is situated within amino acid residues 109-123 (SEQ ID NO: 133) of human alpha-synuclein of SEQ ID NO: 1; or
zz. the first epitope is situated within amino acid residues 124-131 (SEQ ID NO: 7) of human alpha-synuclein of SEQ ID NO: 1 and the second epitope is situated within amino acid residues 1-15 (SEQ ID NO: 121) of human alpha-synuclein of SEQ ID NO: 1; or
aaa. the first epitope is situated within amino acid residues 28-50 (SEQ ID NO: 139) of human alpha-synuclein of SEQ ID NO: 1 and the second epitope is situated within amino acid residues 124-131 (SEQ ID NO: 7) of human alpha-synuclein of SEQ ID NO: 1.

70. The alpha-synuclein biparatopic antibody or functional fragment thereof, or a mixture comprising at least two monospecific antibodies or functional fragments thereof, according to claim 65, comprising a first binding site which binds a first epitope and a second distinct binding site which binds to a second distinct epitope, wherein:
a. the first epitope is situated within amino acid residues 28-42 (SEQ ID NO: 124) of human alpha-synuclein of SEQ ID NO: 1 and the second epitope is situated within amino acid residues 28-50 (SEQ ID NO: 139) of human alpha-synuclein of SEQ ID NO: 1; or
b. the first epitope is situated within amino acid residues 37-51 (SEQ ID NO: 125) of human alpha-synuclein of SEQ ID NO: 1 and the second epitope is situated within amino acid residues 28-50 (SEQ ID NO: 139) of human alpha-synuclein of SEQ ID NO: 1; or
c. the first epitope is situated within amino acid residues 28-42 (SEQ ID NO: 124) and 37-51 (SEQ ID NO: 125) of human alpha-synuclein of SEQ ID NO: 1 and the second epitope is situated within amino acid residues 28-50 (SEQ ID NO: 139) of human alpha-synuclein of SEQ ID NO: 1; or
d. the first epitope is situated within amino acid residues 28-42 (SEQ ID NO: 124) of human alpha-synuclein of SEQ ID NO: 1 and the second epitope is situated within amino acid residues 100-114 (SEQ ID NO: 132) of human alpha-synuclein of SEQ ID NO: 1; or
e. the first epitope is situated within amino acid residues 37-51 (SEQ ID NO: 125) of human alpha-synuclein of SEQ ID NO: 1 and the second epitope is situated within amino acid residues 100-114 (SEQ ID NO: 132) of human alpha-synuclein of SEQ ID NO: 1; or
f. the first epitope is situated within amino acid residues 28-42 (SEQ ID NO: 124) and 37-51 (SEQ ID NO: 125) of human alpha-synuclein of SEQ ID NO: 1 and the second epitope is situated within amino acid residues 100-114 (SEQ ID NO: 132) of human alpha-synuclein of SEQ ID NO: 1; or
g. the first epitope is situated within amino acid residues 100-114 (SEQ ID NO: 132) of human alpha-synuclein of SEQ ID NO: 1 and the second epitope is situated within amino acid residues 28-50 (SEQ ID NO: 139) of human alpha-synuclein of SEQ ID NO: 1; or
h. the first epitope is situated within amino acid residues 109-123 (SEQ ID NO: 133) of human alpha-synuclein of SEQ ID NO: 1 and the second epitope is situated within amino acid residues 28-50 (SEQ ID NO: 139) of human alpha-synuclein of SEQ ID NO: 1; or
i. the first epitope is situated within amino acid residues 100-114 (SEQ ID NO: 132) and 109-123 (SEQ ID NO: 133) of human alpha-synuclein of SEQ ID NO: 1 and the second epitope is situated within amino acid residues 28-50 (SEQ ID NO: 139) of human alpha-synuclein of SEQ ID NO: 1; or
j. the first epitope is situated within amino acid residues 100-114 (SEQ ID NO: 132) of human alpha-synuclein of SEQ ID NO: 1 and the second epitope is situated within amino acid residues 100-114 (SEQ ID NO: 132) of human alpha-synuclein of SEQ ID NO: 1; or
k. the first epitope is situated within amino acid residues 109-123 (SEQ ID NO: 133) of human alpha-synuclein of SEQ ID NO: 1 and the second epitope is situated within amino acid residues 100-114 (SEQ ID NO: 132) of human alpha-synuclein of SEQ ID NO: 1; or
l. the first epitope is situated within amino acid residues 100-114 (SEQ ID NO: 132) and 109-123 (SEQ ID NO: 133) of human alpha-synuclein of SEQ ID NO: 1 and the second epitope is situated within amino acid residues 100-114 (SEQ ID NO: 132) of human alpha-synuclein of SEQ ID NO: 1; or
m. the first epitope is situated within amino acid residues 91-105 (SEQ ID NO: 131) of human alpha-synuclein of SEQ ID NO: 1 and the second epitope is situated within amino acid residues 1-15 (SEQ ID NO: 121) of human alpha-synuclein of SEQ ID NO: 1; or
n. the first epitope is situated within amino acid residues 124-131 (SEQ ID NO: 7) of human alpha-synuclein of SEQ ID NO: 1 and the second epitope is situated within amino acid residues 100-114 (SEQ ID NO: 132) of human alpha-synuclein of SEQ ID NO: 1; or
o. the first epitope is situated within amino acid residues 124-131 (SEQ ID NO: 7) of human alpha-synuclein of SEQ ID NO: 1 and the second epitope is situated within amino acid residues 109-123 (SEQ ID NO: 133) of human alpha-synuclein of SEQ ID NO: 1; or
p. the first epitope is situated within amino acid residues 124-131 (SEQ ID NO: 7) of human alpha-synuclein of SEQ ID NO: 1 and the second epitope is situated within amino acid residues 100-114 (SEQ ID NO: 132) and 109-123 (SEQ ID NO: 133) of human alpha-synuclein of SEQ ID NO: 1; or
q. the first epitope is situated within amino acid residues 82-96 (SEQ ID NO: 130) of human alpha-synuclein of SEQ ID NO: 1 and the second epitope is situated within amino acid residues 124-131 (SEQ ID NO: 7) of human alpha-synuclein of SEQ ID NO: 1; or
r. the first epitope is situated within amino acid residues 81-120 (SEQ ID NO: 137) of human alpha-synuclein of SEQ ID NO: 1 and the second epitope is situated within amino acid residues 28-42 (SEQ ID NO: 124) of human alpha-synuclein of SEQ ID NO: 1; or
s. the first epitope is situated within amino acid residues 81-120 (SEQ ID NO: 137) of human alpha-synuclein of SEQ ID NO: 1 and the second epitope is situated within amino acid residues 37-51 (SEQ ID NO: 125) of human alpha-synuclein of SEQ ID NO: 1; or
t. the first epitope is situated within amino acid residues 81-120 (SEQ ID NO: 137) of human alpha-synuclein of SEQ ID NO: 1 and the second epitope is situated within amino acid residues 28-42 (SEQ ID NO: 124) and 37-51 (SEQ ID NO: 125) of human alpha-synuclein of SEQ ID NO: 1; or
u. the first epitope is situated within amino acid residues 28-50 (SEQ ID NO: 139) of human alpha-synuclein of SEQ ID NO: 1 and the second epitope is situated within amino acid residues 124-131 (SEQ ID NO: 7) of human alpha-synuclein of SEQ ID NO: 1; or
v. the first epitope is situated within amino acid residues 91-105 (SEQ ID NO: 131) of human alpha-synuclein of SEQ ID NO: 1 and the second epitope is situated within amino acid residues 124-131 (SEQ ID NO: 7) of human alpha-synuclein of SEQ ID NO: 1.

71. The alpha-synuclein biparatopic antibody or functional fragment thereof, or a mixture comprising at least two monospecific antibodies or functional fragments thereof, according to claim 65, comprising at least one pair of variable regions Heavy Chain Variable Region (VH) and Light Chain Variable Region (VL), wherein:
a. the VH has at least 96%, 97%, 98%, 99% or 100% sequence identity to the amino acid sequence of SEQ ID NO: 10; and the VL has at least 99% or 100% sequence identity to the amino acid sequence of SEQ ID NO: 14; or
b. the VH has at least 96%, 97%, 98%, 99% or 100% sequence identity to the amino acid sequence of SEQ ID NO: 20; and the VL has at least 93%, 94%, 95%, 96%, 97%, 98%, 99% or 100% sequence identity to the amino acid sequence of SEQ ID NO: 24; or
c. the VH having at least 94%, 95%, 96%, 97%, 98%, 99% or 100% sequence identity to the amino acid sequence of SEQ ID NO: 30; and the VL has at least 98%, 99% or 100% sequence identity to the amino acid sequence of SEQ ID NO: 34; or
d. the VH has at least 94%, 95%, 96%, 97%, 98%, 99% or 100% sequence identity to the amino acid sequence of SEQ ID NO: 40; and the VL has at least 94%, 95%, 96%, 97%, 98%, 99% or 100% sequence identity to the amino acid sequence of SEQ ID NO: 44; or
e. the VH having at least 90%, 91%, 92%, 93%, 94%, 95%, 96%, 97%, 98%, 99% or 100% sequence identity to the amino acid sequence of SEQ ID NO: 50; and the VL has at least 93%, 94%, 95%, 96%, 97%, 98%, 99% or 100% sequence identity to the amino acid sequence of SEQ ID NO: 54; or
f. the VH has at least 89%, 90%, 91%, 92%, 93%, 94%, 95%, 96%, 97%, 98%, 99% or 100% sequence identity to the amino acid sequence of SEQ ID NO: 60; and the VL has at least 96%, 97%, 98%, 99% or 100% sequence identity to the amino acid sequence of SEQ ID NO: 64; or
g. the VH has at least 91%, 92%, 93%, 94%, 95%, 96%, 97%, 98%, 99% or 100% sequence identity to the amino acid sequence of SEQ ID NO: 70; and the VL has at least 90%, 91%, 92%, 93%, 94%, 95%, 96%, 97%, 98%, 99% or 100% sequence identity to the amino acid sequence of SEQ ID NO: 74; or
h. the VH has at least 94%, 95%, 96%, 97%, 98%, 99% or 100% sequence identity to the amino acid sequence of SEQ ID NO: 30; and the VL has at least 94%, 95%, 96%, 97%, 98%, 99% or 100% sequence identity to the amino acid sequence of SEQ ID NO: 84; or
i. the VH has at least 94%, 95%, 96%, 97%, 98%, 99% or 100% sequence identity to the amino acid sequence of SEQ ID NO: 90; and the VL has at least 99% or 100% sequence identity to the amino acid sequence of SEQ ID NO: 94; or
j. the VH has at least 87%, 88%, 89%, 90%, 91%, 92%, 93%, 94%, 95%, 96%, 97%, 98%, 99% or 100% sequence identity to the amino acid sequence of SEQ ID NO: 100; and the VL has at least 98%, 99% or 100% sequence identity to the amino acid sequence of SEQ ID NO: 104; or
k. the VH has at least 97%, 98%, 99% or 100% sequence identity to the amino acid sequence of SEQ ID NO: 110; and the VL comprises the sequence of SEQ ID NO: 114: or
l. the VH has at least 91%, 92%, 93%, 94%, 95%, 96%, 97%, 98%, 99% or 100% sequence identity to the amino acid sequence of SEQ ID NO: 280; and the VL comprises the sequence of SEQ ID NO: 284; or
m. the VH has at least 91%, 92%, 93%, 94%, 95%, 96%, 97%, 98%, 99% or 100% sequence identity to the amino acid sequence of SEQ ID NO: 290; and the VL has at least 95%, 96%, 97%, 98%, 99% or 100% sequence identity to the amino acid sequence of SEQ ID NO: 194; or
n. the VH has at least 94%, 95%, 96%, 97%, 98%, 99% or 100% sequence identity to the amino acid sequence of SEQ ID NO: 140; and the VL has at least 97%, 98%, 99%, 99% or 100% sequence identity to the amino acid sequence of SEQ ID NO: 144; or
o. the VH has at least 90%, 91%, 92%, 93%, 94%, 95%, 96%, 97%, 98%, 99% or 100% sequence identity to the amino acid sequence of SEQ ID NO: 150; and the VL has at least 98%, 99% or 100% sequence identity to the amino acid sequence of SEQ ID NO: 154; or
p. the VH has at least 97%, 98%, 99% or 100% sequence identity to the amino acid sequence of SEQ ID NO: 160; and the VL comprises the sequence of SEQ ID NO: 164; or
q. the VH has at least 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 170; and the VL has at least 93%, 94%, 95%, 96%, 97%, 98%, 99% or 100% sequence identity to the amino acid sequence of SEQ ID NO: 174; or
r. the VH has at least 93%, 94%, 95%, 96%, 97%, 98%, 99% or 100% sequence identity to the amino acid sequence of SEQ ID NO: 180; and the VL comprises the sequence of SEQ ID NO: 184; or
s. the VH has at least 88%, 89%, 90%, 91%, 92%, 93%, 94%, 95%, 96%, 97%, 98%, 99% or 100% sequence identity to the amino acid sequence of SEQ ID NO: 190; and the VL has at least 95%, 96%, 97%, 98%, 99% or 100% sequence identity to the amino acid sequence of SEQ ID NO: 194; or
t. the VH has at least 93%, 94%, 95%, 96%, 97%, 98%, 99% or 100% sequence identity to the amino acid sequence of SEQ ID NO: 200; and the VL has at least 97%, 98%, 99% or 100% sequence identity to the amino acid sequence of SEQ ID NO: 204; or
u. the VH has at least 91%, 92%, 93%, 94%, 95%, 96%, 97%, 98%, 99% or 100% sequence identity to the amino acid sequence of SEQ ID NO: 210; and the VL has at least 97%, 98%, 99% or 100% sequence identity to the amino acid sequence of SEQ ID NO: 214; or
v. the VH has at least 97%, 98%, 99% or 100% sequence identity to the amino acid sequence of SEQ ID NO: 220; and the VL having at least 92%, 93%, 94%, 95%, 96%, 97%, 98%, 99% or 100% sequence identity to the amino acid sequence of SEQ ID NO: 224; or
w. the VH has at least 90%, 91%, 92%, 93%, 94%, 95%, 96%, 97%, 98%, 99% or 100% sequence identity to the amino acid sequence of SEQ ID NO: 230; and the VL has at least 97%, 98%, 99% or 100% sequence identity to the amino acid sequence of SEQ ID NO: 234; or
x. the VH has at least 96%, 97%, 98%, 99% or 100% sequence identity to the amino acid sequence of SEQ ID NO: 240; and the VL has at least 94%, 95%, 96%, 97%, 98%, 99% or 100% sequence identity to the amino acid sequence of SEQ ID NO: 244; or
y. the VH has at least 90%, 91%, 92%, 93%, 94%, 95%, 96%, 97%, 98%, 99% or 100% sequence identity to the amino acid sequence of SEQ ID NO: 250; and the VL has at least 94%, 95%, 96%, 97%, 98%, 99% or 100% sequence identity to the amino acid sequence of SEQ ID NO: 254; or
z. the VH has at least 92%, 93%, 94%, 95%, 96%, 97%, 98%, 99% or 100% sequence identity to the amino acid sequence of SEQ ID NO: 260; and the VL has at least 96%, 97%, 98%, 99% or 100% sequence identity to the amino acid sequence of SEQ ID NO: 264; or
aa. the VH has at least 85%, 86%, 87%, 88%, 89%, 90%, 91%, 92%, 93%, 94%, 95%, 96%, 97%, 98%, 99% or 100% sequence identity to the amino acid sequence of SEQ ID NO: 270; and the VL has at least 91%, 92%, 93%, 94%, 95%, 96%, 97%, 98%, 99% or 100% sequence identity to the amino acid sequence of SEQ ID NO: 274; or
bb. the VH has at least 97%, 98%, 99% or 100% sequence identity to the amino acid sequence of SEQ ID NO: 300; and the VL has at least 97%, 98%, 99% or 100% sequence identity to the amino acid sequence of SEQ ID NO: 304; or
cc. the VH has at least 94%, 95%, 96%, 97%, 98%, 99% or 100% sequence identity to the amino acid sequence of SEQ ID NO: 310; and the VL has at least 96%, 97%, 98%, 99% or 100% sequence identity to the amino acid sequence of SEQ ID NO: 314; or
dd. the VH has at least 94%, 95%, 96%, 97%, 98%, 99% or 100% sequence identity to the amino acid sequence of SEQ ID NO: 320; and the VL has at least 99% or 100% sequence identity to the amino acid sequence of SEQ ID NO: 324; or
ee. the VH has at least 96%, 97%, 98%, 99% or 100% sequence identity to the amino acid sequence of SEQ ID NO: 330; and the VL has at least 97%, 98%, 99% or 100% sequence identity to the amino acid sequence of SEQ ID NO: 334; or
ff. the VH has at least 93%, 94%, 95%, 96%, 97%, 98%, 99% or 100% sequence identity to the amino acid sequence of SEQ ID NO: 340; and the VL has at least 98%, 99% or 100% sequence identity to the amino acid sequence of SEQ ID NO: 344; or
gg. the VH has at least 92%, 93%, 94%, 95%, 96%, 97%, 98%, 99% or 100% sequence identity to the amino acid sequence of SEQ ID NO: 350; and the VL has at least 95%, 96%, 97%, 98%, 99% or 100% sequence identity to the amino acid sequence of SEQ ID NO: 354; or
hh. the VH has at least 93%, 94%, 95%, 96%, 97%, 98%, 99% or 100% sequence identity to the amino acid sequence of SEQ ID NO: 360; and the VL has at least 99% or 100% sequence identity to the amino acid sequence of SEQ ID NO: 364; or
ii. the VH has at least 90%, 91%, 92%, 93%, 94%, 95%, 96%, 97%, 98%, 99% or 100% sequence identity to the amino acid sequence of SEQ ID NO: 370; and the VL has at least 98%, 99% or 100% sequence identity to the amino acid sequence of SEQ ID NO: 374; or
jj. the VH has at least 92%, 93%, 94%, 95%, 96%, 97%, 98%, 99% or 100% sequence identity to the amino acid sequence of SEQ ID NO: 380; and the VL has at least 95%, 96%, 97%, 98%, 99% or 100% sequence identity to the amino acid sequence of SEQ ID NO: 384; or
kk. the VH has at least 91%, 92%, 93%, 94%, 95%, 96%, 97%, 98%, 99% or 100% sequence identity to the amino acid sequence of SEQ ID NO: 390; and the VL has at least 96%, 97%, 98%, 99% or 100% sequence identity to the amino acid sequence of SEQ ID NO: 394; or
ll. the VH has at least 93%, 94%, 95%, 96%, 97%, 98%, 99% or 100% sequence identity to the amino acid sequence of SEQ ID NO: 400; and the VL has at least 93%, 94%, 95%, 96%, 97%, 98%, 99% or 100% sequence identity to the amino acid sequence of SEQ ID NO: 404; or
mm. the VH has at least 92%, 93%, 94%, 95%, 96%, 97%, 98%, 99% or 100% sequence identity to the amino acid sequence of SEQ ID NO: 410; and the VL comprises the amino acid sequence of SEQ ID NO: 414; or
nn. the VH has at least 94%, 95%, 96%, 97%, 98%, 99% or 100% sequence identity to the amino acid sequence of SEQ ID NO: 420; and the VL has at least 99% or 100% sequence identity to the amino acid sequence of SEQ ID NO: 424; or
oo. the VH has at least 96%, 97%, 98%, 99% or 100% sequence identity to the amino acid sequence of SEQ ID NO: 430; and the VL has at least 98%, 99% or 100% sequence identity to the amino acid sequence of SEQ ID NO: 434; or
pp. the VH has at least 93%, 94%, 95%, 96%, 97%, 98%, 99% or 100% sequence identity to the amino acid sequence of SEQ ID NO: 440; and the VL comprises the amino acid sequence of SEQ ID NO: 414; or
qq. the VH has at least 93%, 94%, 95%, 96%, 97%, 98%, 99% or 100% sequence identity to the amino acid sequence of SEQ ID NO: 450; and the VL has at least 99% or 100% sequence identity to the amino acid sequence of SEQ ID NO: 424; or
rr. the VH has at least 94%, 95%, 96%, 97%, 98%, 99% or 100% sequence identity to the amino acid sequence of SEQ ID NO: 460; and the VL has at least 99% or 100% sequence identity to the amino acid sequence of SEQ ID NO: 464; or
ss. the VH has at least 96%, 97%, 98%, 99% or 100% sequence identity to the amino acid sequence of SEQ ID NO: 470; and the VL has at least 99% or 100% sequence identity to the amino acid sequence of SEQ ID NO: 474; or
tt. the VH has at least 98%, 99% or 100% sequence identity to the amino acid sequence of SEQ ID NO: 480; and the VL comprises the amino acid sequence of SEQ ID NO: 484; or
uu. the VH has at 98%, 99% or 100% sequence identity to the amino acid sequence of SEQ ID NO: 490; and the VL has at least 99% or 100% sequence identity to the amino acid sequence of SEQ ID NO: 494; or
vv. the VH has at least 86%, 87%, 88%, 89%, 90%, 91%, 92%, 93%, 94%, 95%, 96%, 97%, 98%, 99% or 100% sequence identity to the amino acid sequence of SEQ ID NO: 500; and the VL has at least 97%, 98%, 99% or 100% sequence identity to the amino acid sequence of SEQ ID NO: 504; or
ww. the VH has at least 88%, 89%, 90%, 91%, 92%, 93%, 94%, 95%, 96%, 97%, 98%, 99% or 100% sequence identity to the amino acid sequence of SEQ ID NO: 510; and the VL has at least 98%, 99% or 100% sequence identity to the amino acid sequence of SEQ ID NO: 514; or
xx. the VH has at least 89%, 90%, 91%, 92%, 93%, 94%, 95%, 96%, 97%, 98%, 99% or 100% sequence identity to the amino acid sequence of SEQ ID NO: 520; and the VL has at least 99% or 100% sequence identity to the amino acid sequence of SEQ ID NO: 524; or
yy. the VH has at least 88%, 89%, 90%, 91%, 92%, 93%, 94%, 95%, 96%, 97%, 98%, 99% or 100% sequence identity to the amino acid sequence of SEQ ID NO: 530; and the VL comprises the amino acid sequence of SEQ ID NO: 534; or
zz. the VH has at least 91%, 92%, 93%, 94%, 95%, 96%, 97%, 98%, 99% or 100% sequence identity to the amino acid sequence of SEQ ID NO: 540; and the VL comprises the amino acid sequence of SEQ ID NO: 544; or
aaa. the VH has at least 89%, 90%, 91%, 92%, 93%, 94%, 95%, 96%, 97%, 98%, 99% or 100% sequence identity to the amino acid sequence of SEQ ID NO: 550; and the VL has at least 99% or 100% sequence identity to the amino acid sequence of SEQ ID NO: 554; or
bbb. the VH has at least 88%, 89%, 90%, 91%, 92%, 93%, 94%, 95%, 96%, 97%, 98%, 99% or 100% sequence identity to the amino acid sequence of SEQ ID NO: 560; and the VL has at least 98%, 99% or 100% sequence identity to the amino acid sequence of SEQ ID NO: 564; or
ccc. the VH has at least 92%, 93%, 94%, 95%, 96%, 97%, 98%, 99% or 100% sequence identity to the amino acid sequence of SEQ ID NO: 570; and the VL has at least 97%, 98%, 99% or 100% sequence identity to the amino acid sequence of SEQ ID NO: 574; or
ddd. the VH has at least 95%, 96%, 97%, 98%, 99% or 100% sequence identity to the amino acid sequence of SEQ ID NO: 580; and the VL has at least 99% or 100% sequence identity to the amino acid sequence of SEQ ID NO: 584; or
eee. the VH has at least 95%, 96%, 97%, 98%, 99% or 100% sequence identity to the amino acid sequence of SEQ ID NO: 590; and the VL has at least 99% or 100% sequence identity to the amino acid sequence of SEQ ID NO: 474; or
fff. the VH has at least 89%, 90%, 91%, 92%, 93%, 94%, 95%, 96%, 97%, 98%, 99% or 100% sequence identity to the amino acid sequence of SEQ ID NO: 600; and the VL has at least 99% or 100% sequence identity to the amino acid sequence of SEQ ID NO: 554; or
ggg. the VH has at least 92%, 93%, 94%, 95%, 96%, 97%, 98%, 99% or 100% sequence identity to the amino acid sequence of SEQ ID NO: 610; and the VL comprises the amino acid sequence of SEQ ID NO: 614; or
hhh. the VH has at least 96%, 97%, 98%, 99% or 100% sequence identity to the amino acid sequence of SEQ ID NO: 620; and the VL has at least 92%, 93%, 94%, 95%, 96%, 97%, 98%, 99% or 100% sequence identity to the amino acid sequence of SEQ ID NO: 624; or
iii. the VH has at 99% or 100% sequence identity to the amino acid sequence of SEQ ID NO: 630; and the VL has at least 99% or 100% sequence identity to the amino acid sequence of SEQ ID NO: 634; or
jjj. the VH has at least 96%, 97%, 98%, 99% or 100% sequence identity to the amino acid sequence of SEQ ID NO: 640; and the VL has at least 96%, 97%, 98%, 99% or 100% sequence identity to the amino acid sequence of SEQ ID NO: 644; or
kkk. the VH has at least 98%, 99% or 100% sequence identity to the amino acid sequence of SEQ ID NO: 650; and the VL has at least 99% or 100% sequence identity to the amino acid sequence of SEQ ID NO: 654; or
lll. the VH has at least 92%, 93%, 94%, 95%, 96%, 97%, 98%, 99% or 100% sequence identity to the amino acid sequence of SEQ ID NO: 660; and the VL has at least 99% or 100% sequence identity to the amino acid sequence of SEQ ID NO: 664; or
mmm. the VH has at least 92%, 93%, 94%, 95%, 96%, 97%, 98%, 99% or 100% sequence identity to the amino acid sequence of SEQ ID NO: 670; and the VL comprises the amino acid sequence of SEQ ID NO: 674; or
nnn. the VH has at least 97%, 98%, 99% or 100% sequence identity to the amino acid sequence of SEQ ID NO: 680; and the VL has at least 99% or 100% sequence identity to the amino acid sequence of SEQ ID NO: 684; or
ooo. the VH has at least 87%, 88%, 89%, 90%, 91%, 92%, 93%, 94%, 95%, 96%, 97%, 98%, 99% or 100% sequence identity to the amino acid sequence of SEQ ID NO: 690; and the VL has at least 96%, 97%, 98%, 99% or 100% sequence identity to the amino acid sequence of SEQ ID NO: 694; or
ppp. the VH has at least 86%, 87%, 88%, 89%, 90%, 91%, 92%, 93%, 94%, 95%, 96%, 97%, 98%, 99% or 100% sequence identity to the amino acid sequence of SEQ ID NO: 700; and the VL has at least 94%, 95%, 96%, 97%, 98%, 99% or 100% sequence identity to the amino acid sequence of SEQ ID NO: 704; or
qqq. the VH has at least 96%, 97%, 98%, 99% or 100% sequence identity to the amino acid sequence of SEQ ID NO: 710; and the VL has at 98%, 99% or 100% sequence identity to the amino acid sequence of SEQ ID NO: 714; or
rrr. the VH comprises the amino acid sequence of SEQ ID NO: 720; and the VL has at least 95%, 96%, 97%, 98%, 99% or 100% sequence identity to the amino acid sequence of SEQ ID NO: 724; or
sss. the VH has at least 97%, 98%, 99% or 100% sequence identity to the amino acid sequence of SEQ ID NO: 730; and the VL has at least 97%, 98%, 99% or 100% sequence identity to the amino acid sequence of SEQ ID NO: 734; or
ttt. the VH has at least 88%, 89%, 90%, 91%, 92%, 93%, 94%, 95%, 96%, 97%, 98%, 99% or 100% sequence identity to the amino acid sequence of SEQ ID NO: 740; and the VL has at least 99% or 100% sequence identity to the amino acid sequence of SEQ ID NO: 744; or
uuu. the VH has at least 99% or 100% sequence identity to the amino acid sequence of SEQ ID NO: 750; and the VL has at least 97%, 98%, 99% or 100% sequence identity to the amino acid sequence of SEQ ID NO: 754; or
vvv. the VH has at least 97%, 98%, 99% or 100% sequence identity to the amino acid sequence of SEQ ID NO: 760; and the VL has at 98%, 99% or 100% sequence identity to the amino acid sequence of SEQ ID NO: 764; or
www. the VH has at least 93%, 94%, 95%, 96%, 97%, 98%, 99% or 100% sequence identity to the amino acid sequence of SEQ ID NO: 770; and the VL has at least 97%, 98%, 99% or 100% sequence identity to the amino acid sequence of SEQ ID NO: 774; or
xxx. the VH has at least 99% or 100% sequence identity to the amino acid sequence of SEQ ID NO: 750; and the VL has at least 96%, 97%, 98%, 99% or 100% sequence identity to the amino acid sequence of SEQ ID NO: 784; or
yyy. the VH has at least 83%, 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 790; and the VL has at least 95%, 96%, 97%, 98%, 99% or 100% sequence identity to the amino acid sequence of SEQ ID NO: 794; or
zzz. the VH has at least 92%, 93%, 94%, 95%, 96%, 97%, 98%, 99% or 100% sequence identity to the amino acid sequence of SEQ ID NO: 800; and the VL has at least 96%, 97%, 98%, 99% or 100% sequence identity to the amino acid sequence of SEQ ID NO: 804; or
aaaa. the VH has at least 94%, 95%, 96%, 97%, 98%, 99% or 100% sequence identity to the amino acid sequence of SEQ ID NO: 810; and the VL has at least 99% or 100% sequence identity to the amino acid sequence of SEQ ID NO: 814; or
bbbb. the VH has at least 98%, 99% or 100% sequence identity to the amino acid sequence of SEQ ID NO: 820; and the VL has at least 98%, 99% or 100% sequence identity to the amino acid sequence of SEQ ID NO: 824; or
cccc. the VH has at least 94%, 95%, 96%, 97%, 98%, 99% or 100% sequence identity to the amino acid sequence of SEQ ID NO: 830; and the VL comprises the amino acid sequence of SEQ ID NO: 834; or
dddd. the VH has at least 90%, 91%, 92%, 93%, 94%, 95%, 96%, 97%, 98%, 99% or 100% sequence identity to the amino acid sequence of SEQ ID NO: 840; and the VL comprises the amino acid sequence of SEQ ID NO: 844.

72. The alpha-synuclein biparatopic antibody or functional fragment thereof, or a mixture comprising at least two monospecific antibodies or functional fragments thereof, according to claim 65, comprising two distinct pairs of variable regions VH and VL, wherein
a. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 50; and a VL which has at least 93%, 94%, 95%, 96%, 97%, 98%, 99% or 100% sequence identity to the amino acid sequence of SEQ ID NO: 54; or
b. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 94%, 95%, 96%, 97%, 98%, 99% or 100% sequence identity to the amino acid sequence of SEQ ID NO: 90; and a VL which has at least 99% or 100% sequence identity to the amino acid sequence of SEQ ID NO: 94; or
c. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 94%, 95%, 96%, 97%, 98%, 99% or 100% sequence identity to the amino acid sequence of SEQ ID NO: 30; and a VL which has at least 94%, 95%, 96%, 97%, 98%, 99% or 100% sequence identity to the amino acid sequence of SEQ ID NO: 84; or
d. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 50; and a VL which has at least 93%, 94%, 95%, 96%, 97%, 98%, 99% or 100% sequence identity to the amino acid sequence of SEQ ID NO: 54; and a second pair of variable regions VH and VL comprising a VH which has at least 94%, 95%, 96%, 97%, 98%, 99% or 100% sequence identity to the amino acid sequence of SEQ ID NO: 90; and a VL which has at least 99% or 100% sequence identity to the amino acid sequence of SEQ ID NO: 94; or
e. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 50; and a VL which has at least 93%, 94%, 95%, 96%, 97%, 98%, 99% or 100% sequence identity to the amino acid sequence of SEQ ID NO: 54; and a second pair of variable regions VH and VL comprising a VH which has at least 94%, 95%, 96%, 97%, 98%, 99% or 100% sequence identity to the amino acid sequence of SEQ ID NO: 30; and a VL which has at least 94%, 95%, 96%, 97%, 98%, 99% or 100% sequence identity to the amino acid sequence of SEQ ID NO: 84; or
f. a first pair of variable regions VH and VL comprising a VH which has at least 93%, 94%, 95%, 96%, 97%, 98%, 99% or 100% sequence identity to the amino acid sequence of SEQ ID NO: 180; and a VL comprises the sequence of SEQ ID NO: 184; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; or
g. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 150; and a VL has at least 98%, 99% or 100% sequence identity to the amino acid sequence of SEQ ID NO: 154; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; or
h. a first pair of variable regions VH and VL comprising a VH which has at least 93%, 94%, 95%, 96%, 97%, 98%, 99% or 100% sequence identity to the amino acid sequence of SEQ ID NO: 180; and a VL comprises the sequence of SEQ ID NO: 184; and a second pair of variable regions VH and VL comprising a VH which has at least 94%, 95%, 96%, 97%, 98%, 99% or 100% sequence identity to the amino acid sequence of SEQ ID NO: 90; and a VL which has at least 99% or 100% sequence identity to the amino acid sequence of SEQ ID NO: 94; or
i. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 150; and a VL has at least 98%, 99% or 100% sequence identity to the amino acid sequence of SEQ ID NO: 154; and a second pair of variable regions VH and VL comprising a VH which has at least 94%, 95%, 96%, 97%, 98%, 99% or 100% sequence identity to the amino acid sequence of SEQ ID NO: 90; and a VL which has at least 99% or 100% sequence identity to the amino acid sequence of SEQ ID NO: 94; or
j. a first pair of variable regions VH and VL comprising a VH which has at least 94%, 95%, 96%, 97%, 98%, 99% or 100% sequence identity to the amino acid sequence of SEQ ID NO: 30; and a VL which has at least 94%, 95%, 96%, 97%, 98%, 99% or 100% sequence identity to the amino acid sequence of SEQ ID NO: 84; and a second pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; or
k. a first pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; and a second pair of variable regions VH and VL comprising a VH which has at least 92%, 93%, 94%, 95%, 96%, 97%, 98%, 99% or 100% sequence identity to the amino acid sequence of SEQ ID NO: 670; and a VL which comprises the amino acid sequence of SEQ ID NO: 674; or
l. a first pair of variable regions VH and VL comprising a VH which has at least 94%, 95%, 96%, 97%, 98%, 99% or 100% sequence identity to the amino acid sequence of SEQ ID NO: 320; and a VL which has at least 99% or 100% sequence identity to the amino acid sequence of SEQ ID NO: 324; and a second pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; or
m. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 50; and a VL which has at least 93%, 94%, 95%, 96%, 97%, 98%, 99% or 100% sequence identity to the amino acid sequence of SEQ ID NO: 54; and a second pair of variable regions VH and VL comprising a VH which has at least 93%, 94%, 95%, 96%, 97%, 98%, 99% or 100% sequence identity to the amino acid sequence of SEQ ID NO: 360; and a VL which has at least 99% or 100% sequence identity to the amino acid sequence of SEQ ID NO: 364; or
n. a first pair of variable regions VH and VL comprising a VH which has at least 93%, 94%, 95%, 96%, 97%, 98%, 99% or 100% sequence identity to the amino acid sequence of SEQ ID NO: 400; and a VL which has at least 93%, 94%, 95%, 96%, 97%, 98%, 99% or 100% sequence identity to the amino acid sequence of SEQ ID NO: 404; and a second pair of variable regions VH and VL comprising a VH which has at least 94%, 95%, 96%, 97%, 98%, 99% or 100% sequence identity to the amino acid sequence of SEQ ID NO: 90; and a VL which has at least 99% or 100% sequence identity to the amino acid sequence of SEQ ID NO: 94; or
o. a first pair of variable regions VH and VL comprising a VH which has at least 88%, 89%, 90%, 91%, 92%, 93%, 94%, 95%, 96%, 97%, 98%, 99% or 100% sequence identity to the amino acid sequence of SEQ ID NO: 530; and a VL which comprises the amino acid sequence of SEQ ID NO: 534; and a second pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; or
p. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 88%, 89%, 90%, 91%, 92%, 93%, 94%, 95%, 96%, 97%, 98%, 99% or 100% sequence identity to the amino acid sequence of SEQ ID NO: 530; and a VL which comprises the amino acid sequence of SEQ ID NO: 534; or
q. a first pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; and a second pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 150; and a VL which has at least 98%, 99% or 100% sequence identity to the amino acid sequence of SEQ ID NO: 154; or
r. a first pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; and a second pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; or
s. a first pair of variable regions VH and VL comprising a VH which has at least 88%, 89%, 90%, 91%, 92%, 93%, 94%, 95%, 96%, 97%, 98%, 99% or 100% sequence identity to the amino acid sequence of SEQ ID NO: 530; and a VL which comprises the amino acid sequence of SEQ ID NO: 534; and a second pair of variable regions VH and VL comprising a VH which has at least 93%, 94%, 95%, 96%, 97%, 98%, 99% or 100% sequence identity to the amino acid sequence of SEQ ID NO: 400; and a VL which has at least 93%, 94%, 95%, 96%, 97%, 98%, 99% or 100% sequence identity to the amino acid sequence of SEQ ID NO: 404; or
t. a first pair of variable regions VH and VL comprising a VH which has at least 94%, 95%, 96%, 97%, 98%, 99% or 100% sequence identity to the amino acid sequence of SEQ ID NO: 320; and a VL which has at least 99% or 100% sequence identity to the amino acid sequence of SEQ ID NO: 324; and a second pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; or
u. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 50; and a VL which has at least 93%, 94%, 95%, 96%, 97%, 98%, 99% or 100% sequence identity to the amino acid sequence of SEQ ID NO: 54; and a second pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; or
v. a first pair of variable regions VH and VL comprising a VH which has at least 92%, 93%, 94%, 95%, 96%, 97%, 98%, 99% or 100% sequence identity to the amino acid sequence of SEQ ID NO: 670; and a VL which comprises the amino acid sequence of SEQ ID NO: 674; and a second pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; or
w. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; or
x. a first pair of variable regions VH and VL comprising a VH which has at least 95%, 96%, 97%, 98%, 99% or 100% sequence identity to the amino acid sequence of SEQ ID NO: 590; and a VL which has at least 99% or 100% sequence identity to the amino acid sequence of SEQ ID NO: 474; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; or
y. a first pair of variable regions VH and VL comprising a VH which has at least 95%, 96%, 97%, 98%, 99% or 100% sequence identity to the amino acid sequence of SEQ ID NO: 590; and a VL which has at least 99% or 100% sequence identity to the amino acid sequence of SEQ ID NO: 474; and a second pair of variable regions VH and VL comprising a VH which has at least 93%, 94%, 95%, 96%, 97%, 98%, 99% or 100% sequence identity to the amino acid sequence of SEQ ID NO: 400; and a VL which has at least 93%, 94%, 95%, 96%, 97%, 98%, 99% or 100% sequence identity to the amino acid sequence of SEQ ID NO: 404; or
z. a first pair of variable regions VH and VL comprising a VH which has at least 95%, 96%, 97%, 98%, 99% or 100% sequence identity to the amino acid sequence of SEQ ID NO: 590; and a VL which has at least 99% or 100% sequence identity to the amino acid sequence of SEQ ID NO: 474; and a second pair of variable regions VH and VL comprising a VH which has at least 94%, 95%, 96%, 97%, 98%, 99% or 100% sequence identity to the amino acid sequence of SEQ ID NO: 320; and a VL which has at least 99% or 100% sequence identity to the amino acid sequence of SEQ ID NO: 324; or
aa. a first pair of variable regions VH and VL comprising a VH which has at least 95%, 96%, 97%, 98%, 99% or 100% sequence identity to the amino acid sequence of SEQ ID NO: 590; and a VL which has at least 99% or 100% sequence identity to the amino acid sequence of SEQ ID NO: 474; and a second pair of variable regions VH and VL comprising a VH which has at least 92%, 93%, 94%, 95%, 96%, 97%, 98%, 99% or 100% sequence identity to the amino acid sequence of SEQ ID NO: 570; and a VL which has at least 97%, 98%, 99% or 100% sequence identity to the amino acid sequence of SEQ ID NO: 574; or
bb. a first pair of variable regions VH and VL comprising a VH which has at least 95%, 96%, 97%, 98%, 99% or 100% sequence identity to the amino acid sequence of SEQ ID NO: 590; and a VL which has at least 99% or 100% sequence identity to the amino acid sequence of SEQ ID NO: 474; and a second pair of variable regions VH and VL comprising a VH which has at least 93%, 94%, 95%, 96%, 97%, 98%, 99% or 100% sequence identity to the amino acid sequence of SEQ ID NO: 340; and a VL which has at least 98%, 99% or 100% sequence identity to the amino acid sequence of SEQ ID NO: 344; or
cc. a first pair of variable regions VH and VL comprising a VH which has at least 94%, 95%, 96%, 97%, 98%, 99% or 100% sequence identity to the amino acid sequence of SEQ ID NO: 30; and a VL which has at least 94%, 95%, 96%, 97%, 98%, 99% or 100% sequence identity to the amino acid sequence of SEQ ID NO: 84; and a second pair of variable regions VH and VL comprising a VH which has at least 95%, 96%, 97%, 98%, 99% or 100% sequence identity to the amino acid sequence of SEQ ID NO: 590; and a VL which has at least 99% or 100% sequence identity to the amino acid sequence of SEQ ID NO: 474; or
dd. a first pair of variable regions VH and VL comprising a VH which has at least 94%, 95%, 96%, 97%, 98%, 99% or 100% sequence identity to the amino acid sequence of SEQ ID NO: 30; and a VL which has at least 94%, 95%, 96%, 97%, 98%, 99% or 100% sequence identity to the amino acid sequence of SEQ ID NO: 84; and a second pair of variable regions VH and VL comprising a VH which has at least 88%, 89%, 90%, 91%, 92%, 93%, 94%, 95%, 96%, 97%, 98%, 99% or 100% sequence identity to the amino acid sequence of SEQ ID NO: 510; and a VL which has at least 98%, 99% or 100% sequence identity to the amino acid sequence of SEQ ID NO: 514; or
ee. a first pair of variable regions VH and VL comprising a VH which has at least 93%, 94%, 95%, 96%, 97%, 98%, 99% or 100% sequence identity to the amino acid sequence of SEQ ID NO: 340; and a VL which has at least 98%, 99% or 100% sequence identity to the amino acid sequence of SEQ ID NO: 344; and a second pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; or
ff. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 93%, 94%, 95%, 96%, 97%, 98%, 99% or 100% sequence identity to the amino acid sequence of SEQ ID NO: 400; and a VL which has at least 93%, 94%, 95%, 96%, 97%, 98%, 99% or 100% sequence identity to the amino acid sequence of SEQ ID NO: 404; or
gg. a first pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; or
hh. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 96%, 97%, 98%, 99% or 100% sequence identity to the amino acid sequence of SEQ ID NO: 330; and a VL which has at least 97%, 98%, 99% or 100% sequence identity to the amino acid sequence of SEQ ID NO: 334; or
ii. a first pair of variable regions VH and VL comprising a VH which has at least 92%, 93%, 94%, 95%, 96%, 97%, 98%, 99% or 100% sequence identity to the amino acid sequence of SEQ ID NO: 670; and a VL which comprises the amino acid sequence of SEQ ID NO: 674; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; or
jj. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 92%, 93%, 94%, 95%, 96%, 97%, 98%, 99% or 100% sequence identity to the amino acid sequence of SEQ ID NO: 610; and a VL which comprises the amino acid sequence of SEQ ID NO: 614.

73. The alpha-synuclein biparatopic antibody or functional fragment thereof, or a mixture comprising at least two monospecific antibodies or functional fragments thereof, according to claim 65, comprising two distinct pairs of variable regions VH and VL, wherein:
a. a first pair of variable regions VH and VL comprising a VH which has at least 94%, 95%, 96%, 97%, 98%, 99% or 100% sequence identity to the amino acid sequence of SEQ ID NO: 320; and a VL which has at least 99% or 100% sequence identity to the amino acid sequence of SEQ ID NO: 324; and a second pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; or
b. a first pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; and a second pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; or
c. a first pair of variable regions VH and VL comprising a VH which has at least 88%, 89%, 90%, 91%, 92%, 93%, 94%, 95%, 96%, 97%, 98%, 99% or 100% sequence identity to the amino acid sequence of SEQ ID NO: 530; and a VL which comprises the amino acid sequence of SEQ ID NO: 534; and a second pair of variable regions VH and VL comprising a VH which has at least 93%, 94%, 95%, 96%, 97%, 98%, 99% or 100% sequence identity to the amino acid sequence of SEQ ID NO: 400; and a VL which has at least 93%, 94%, 95%, 96%, 97%, 98%, 99% or 100% sequence identity to the amino acid sequence of SEQ ID NO: 404; or
d. a first pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; and a second pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; or
e. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 150; and a VL has at least 98%, 99% or 100% sequence identity to the amino acid sequence of SEQ ID NO: 154; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; or
f. a first pair of variable regions VH and VL comprising a VH which has at least 95%, 96%, 97%, 98%, 99% or 100% sequence identity to the amino acid sequence of SEQ ID NO: 590; and a VL which has at least 99% or 100% sequence identity to the amino acid sequence of SEQ ID NO: 474; and a second pair of variable regions VH and VL comprising a VH which has at least 94%, 95%, 96%, 97%, 98%, 99% or 100% sequence identity to the amino acid sequence of SEQ ID NO: 320; and a VL which has at least 99% or 100% sequence identity to the amino acid sequence of SEQ ID NO: 324; or
g. a first pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14; or
h. a first pair of variable regions VH and VL comprising a VH which has at least 92%, 93%, 94%, 95%, 96%, 97%, 98%, 99% or 100% sequence identity to the amino acid sequence of SEQ ID NO: 670; and a VL which comprises the amino acid sequence of SEQ ID NO: 674; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14.

74. The alpha-synuclein biparatopic antibody or functional fragment thereof, or a mixture comprising at least two monospecific antibodies or functional fragments thereof, according to claim 65, comprising two distinct pairs of variable regions VH and VL, wherein:
a. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 50 and SEQ ID NO: 54; or
b. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 90 and SEQ ID NO: 94; or
c. a first pair of variable regions VH and VL comprises a VH and VL respectively, set forth in SEQ ID NO: 10 and SEQ ID NO 14; and a second pair of variable regions VH and VL comprises a VH and VL respectively, set forth in SEQ ID NO: 30 and SEQ ID NO: 84; or
d. a first pair of variable regions VH and VL comprises a VH and VL respectively, set forth in SEQ ID NO: 50 and SEQ ID NO: 54; and a second pair of variable regions VH and VL comprises a VH and VL respectively, set forth in SEQ ID NO: 90 and SEQ ID NO: 94; or
e. a first pair of variable regions VH and VL comprises a VH and VL respectively, set forth in SEQ ID NO: 50 and SEQ ID NO: 54; and a second pair of variable regions VH and VL comprises a VH and VL respectively, set forth in SEQ ID NO: 30 and SEQ ID NO: 84; or
f. a first pair of variable regions VH and VL comprises a VH and VL respectively, set forth in SEQ ID NO: 180 and SEQ ID NO: 184; and a second pair of variable regions VH and VL comprises a VH and VL respectively, set forth in SEQ ID NO: 10 and SEQ ID NO: 14; or
g. a first pair of variable regions VH and VL comprises a VH and VL respectively, set forth in SEQ ID NO: 150 and SEQ ID NO: 154; and a second pair of variable regions VH and VL comprises a VH and VL respectively, set forth in SEQ ID NO: 10 and SEQ ID NO: 14; or
h. a first pair of variable regions VH and VL comprises a VH and VL respectively, set forth in SEQ ID NO: 180 and SEQ ID NO: 184; and a second pair of variable regions VH and VL comprises a VH and VL respectively, set forth in SEQ ID NO: 90 and SEQ ID NO: 94; or
i. a first pair of variable regions VH and VL comprises a VH and VL respectively, set forth in SEQ ID NO: 150 and SEQ ID NO: 154; and a second pair of variable regions VH and VL comprises a VH and VL respectively, set forth in SEQ ID NO: 90 and SEQ ID NO: 94; or
j. a first pair of variable regions VH and VL comprises a VH and VL respectively, set forth in SEQ ID NO: 30 and SEQ ID NO: 84; and a second pair of variable regions VH and VL comprises a VH and VL respectively, set forth in SEQ ID NO: 690 and SEQ ID NO: 694; or
k. a first pair of variable regions VH and VL comprises a VH and VL respectively, set forth in SEQ ID NO: 690 and SEQ ID NO: 694; and a second pair of variable regions VH and VL comprises a VH and VL respectively, set forth in SEQ ID NO: 670 and SEQ ID NO: 674; or
l. a first pair of variable regions VH and VL comprises a VH and VL respectively, set forth in SEQ ID NO: 320 and SEQ ID NO: 324; and a second pair of variable regions VH and VL comprises a VH and VL respectively, set forth in SEQ ID NO: 690 and SEQ ID NO: 694; or
m. a first pair of variable regions VH and VL comprises a VH and VL respectively, set forth in SEQ ID NO: 50 and SEQ ID NO: 54; and a second pair of variable regions VH and VL comprises a VH and VL respectively, set forth in SEQ ID NO: 360 and SEQ ID NO: 364; or
n. a first pair of variable regions VH and VL comprises a VH and VL respectively, set forth in SEQ ID NO: 400 and SEQ ID NO: 404; and a second pair of variable regions VH and VL comprises a VH and VL respectively, set forth in SEQ ID NO: 90 and SEQ ID NO: 94; or
o. a first pair of variable regions VH and VL comprises a VH and VL respectively, set forth in SEQ ID NO: 530 and SEQ ID NO: 534; and a second pair of variable regions VH and VL comprises a VH and VL respectively, set forth in SEQ ID NO: 460 and SEQ ID NO: 464; or
p. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 530 and SEQ ID NO: 534; or
q. a first pair of variable regions VH and VL comprises a VH and VL respectively, set forth in SEQ ID NO: 460 and SEQ ID NO: 464; and a second pair of variable regions VH and VL comprises a VH and VL respectively, set forth in SEQ ID NO: 150 and SEQ ID NO: 154; or
r. a first pair of variable regions VH and VL comprises a VH and VL respectively, set forth in SEQ ID NO: 460 and SEQ ID NO: 464; and a second pair of variable regions VH and VL comprises a VH and VL respectively, set forth in SEQ ID NO: 690 and SEQ ID NO: 694; or
s. a first pair of variable regions VH and VL comprises a VH and VL respectively, set forth in SEQ ID NO: 530 and SEQ ID NO: 534; and a second pair of variable regions VH and VL comprises a VH and VL respectively, set forth in SEQ ID NO: 400 and SEQ ID NO: 404; or
t. a first pair of variable regions VH and VL comprises a VH and VL respectively, set forth in SEQ ID NO: 320 and SEQ ID NO: 324; and a second pair of variable regions VH and VL comprises a VH and VL respectively, set forth in SEQ ID NO: 460 and SEQ ID NO: 464; or
u. a first pair of variable regions VH and VL comprises a VH and VL respectively, set forth in SEQ ID NO: 50 and SEQ ID NO: 54; and a second pair of variable regions VH and VL comprises a VH and VL respectively, set forth in SEQ ID NO: 460 and SEQ ID NO: 464; or
v. a first pair of variable regions VH and VL comprises a VH and VL respectively, set forth in SEQ ID NO: 670 and SEQ ID NO: 674; and a second pair of variable regions VH and VL comprises a VH and VL respectively, set forth in SEQ ID NO: 460 and SEQ ID NO: 464; or
w. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 460 and SEQ ID NO: 464; or
x. a first pair of variable regions VH and VL comprises a VH and VL respectively, set forth in SEQ ID NO: 590 and SEQ ID NO: 474; and a second pair of variable regions VH and VL comprises a VH and VL respectively, set forth in SEQ ID NO: 10 and SEQ ID NO: 14; or
y. a first pair of variable regions VH and VL comprises a VH and VL respectively, set forth in SEQ ID NO: 590 and SEQ ID NO: 474; and a second pair of variable regions VH and VL comprises a VH and VL respectively, set forth in SEQ ID NO: 400 and SEQ ID NO: 404; or
z. a first pair of variable regions VH and VL comprises a VH and VL respectively, set forth in SEQ ID NO: 590 and SEQ ID NO: 474; and a second pair of variable regions VH and VL comprises a VH and VL respectively, set forth in SEQ ID NO: 320 and SEQ ID NO: 324; or
aa. a first pair of variable regions VH and VL comprises a VH and VL respectively, set forth in SEQ ID NO: 590 and SEQ ID NO: 474; and a second pair of variable regions VH and VL comprises a VH and VL respectively, set forth in SEQ ID NO: 570 and SEQ ID NO: 574; or
bb. a first pair of variable regions VH and VL comprises a VH and VL respectively, set forth in SEQ ID NO: 590 and SEQ ID NO: 474; and a second pair of variable regions VH and VL comprises a VH and VL respectively, set forth in SEQ ID NO: 340 and SEQ ID NO: 344; or
cc. a first pair of variable regions VH and VL comprises a VH and VL respectively, set forth in SEQ ID NO: 30 and SEQ ID NO: 84; and a second pair of variable regions VH and VL comprises a VH and VL respectively, set forth in SEQ ID NO: 590 and SEQ ID NO: 474; or
dd. a first pair of variable regions VH and VL comprises a VH and VL respectively, set forth in SEQ ID NO: 30 and SEQ ID NO: 84; and a second pair of variable regions VH and VL comprises a VH and VL respectively, set forth in SEQ ID NO: 510 and SEQ ID NO: 514; or
ee. a first pair of variable regions VH and VL comprises a VH and VL respectively, set forth in SEQ ID NO: 340 and SEQ ID NO: 344; and a second pair of variable regions VH and VL comprises a VH and VL respectively, set forth in SEQ ID NO: 690 and SEQ ID NO: 694; or
ff. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 400 and SEQ ID NO: 404; or
gg. a first pair of variable regions VH and VL comprises a VH and VL respectively, set forth in SEQ ID NO: 690 and SEQ ID NO: 694; and a second pair of variable regions VH and VL comprises a VH and VL respectively, set forth in SEQ ID NO: 10 and SEQ ID NO: 14; or
hh. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 330 and SEQ ID NO: 334; or
ii. a first pair of variable regions VH and VL comprises a VH and VL respectively, set forth in SEQ ID NO: 670 and SEQ ID NO: 674; and a second pair of variable regions VH and VL comprises a VH and VL respectively, set forth in SEQ ID NO: 10 and SEQ ID NO: 14; or
jj. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 610 and SEQ ID NO: 614.

75. The alpha-synuclein biparatopic antibody or functional fragment thereof, or a mixture comprising at least two monospecific antibodies or functional fragments thereof, according to claim 65, comprising two distinct pairs of variable regions VH and VL, wherein:
a. a first pair of variable regions VH and VL comprises a VH and VL respectively, set forth in SEQ ID NO: 320 and SEQ ID NO: 324; and a second pair of variable regions VH and VL comprises a VH and VL respectively, set forth in SEQ ID NO: 690 and SEQ ID NO: 694; or
b. a first pair of variable regions VH and VL comprises a VH and VL respectively, set forth in SEQ ID NO: 460 and SEQ ID NO: 464; and a second pair of variable regions VH and VL comprises a VH and VL respectively, set forth in SEQ ID NO: 690 and SEQ ID NO: 694; or
c. a first pair of variable regions VH and VL comprises a VH and VL respectively, set forth in SEQ ID NO: 530 and SEQ ID NO: 534; and a second pair of variable regions VH and VL comprises a VH and VL respectively, set forth in SEQ ID NO: 400 and SEQ ID NO: 404; or
d. a first pair of variable regions VH and VL comprises a VH and VL respectively, set forth in SEQ ID NO: 10 and SEQ ID NO: 14; and a second pair of variable regions VH and VL comprises a VH and VL respectively, set forth in SEQ ID NO: 460 and SEQ ID NO: 464; or
e. a first pair of variable regions VH and VL comprises a VH and VL respectively, set forth in SEQ ID NO: 150 and SEQ ID NO: 154; and a second pair of variable regions VH and VL comprises a VH and VL respectively, set forth in SEQ ID NO: 10 and SEQ ID NO: 14; or
f. a first pair of variable regions VH and VL comprises a VH and VL respectively, set forth in SEQ ID NO: 590 and SEQ ID NO: 474; and a second pair of variable regions VH and VL comprises a VH and VL respectively, set forth in SEQ ID NO: 320 and SEQ ID NO: 324; or
g. a first pair of variable regions VH and VL comprises a VH and VL respectively, set forth in SEQ ID NO: 690 and SEQ ID NO: 694; and a second pair of variable regions VH and VL comprises a VH and VL respectively, set forth in SEQ ID NO: 10 and SEQ ID NO: 14; or
h. a first pair of variable regions VH and VL comprises a VH and VL respectively, set forth in SEQ ID NO: 670 and SEQ ID NO: 674; and a second pair of variable regions VH and VL comprises a VH and VL respectively, set forth in SEQ ID NO: 10 and SEQ ID NO: 14.

76. The alpha-synuclein biparatopic antibody or functional fragment thereof, or a mixture comprising at least two monospecific antibodies or functional fragments thereof, according to claim 65, comprising at least one pair of variable regions Heavy Chain Variable Region (VH) and Light Chain Variable Region (VL), wherein:
a. a heavy chain variable region (VH) comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a light chain variable region (VL) comprises a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b. a heavy chain variable region (VH) comprises a VH-CDR1 comprising the amino acid sequence of SEQ ID NO: 21; a VH-CDR2 comprising the amino acid sequence of SEQ ID NO: 22; a VH-CDR3 comprising the amino acid sequence YSY; a light chain variable region (VL) comprises a VL-CDR1 comprising the amino acid sequence of SEQ ID NO: 25; a VL-CDR2 comprising the amino acid sequence of SEQ ID NO: 26; and a VL-CDR3 comprising the amino acid sequence of SEQ ID NO: 27; or
c. a heavy chain variable region (VH) comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a light chain variable region (VL) comprising a VL-CDR1 comprises the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
d. a heavy chain variable region (VH) comprises a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a light chain variable region (VL) comprises a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
e. a heavy chain variable region (VH) comprising a VH-CDR1 comprising the amino acid sequence of SEQ ID NO: 21; a VH-CDR2 comprises the amino acid sequence of SEQ ID NO: 52; a VH-CDR3 comprising the amino acid sequence YSF; a light chain variable region (VL) comprises a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; or
f. a heavy chain variable region (VH) comprises a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 43; a light chain variable region (VL) comprises a VL-CDR1 comprising the amino acid sequence of SEQ ID NO: 65; a VL-CDR2 comprising the amino acid sequence of SEQ ID NO: 46; and a VL-CDR3 comprising the amino acid sequence of SEQ ID NO: 67; or
g. a heavy chain variable region (VH) comprises a VH-CDR1 comprising the amino acid sequence of SEQ ID NO: 21; a VH-CDR2 comprising the amino acid sequence of SEQ ID NO: 72; a VH-CDR3 comprising the amino acid sequence YSY; a light chain variable region (VL) comprises a VL-CDR1 comprising the amino acid sequence of SEQ ID NO: 75; a VL-CDR2 comprising the amino acid sequence of SEQ ID NO: 76; and a VL-CDR3 comprising the amino acid sequence of SEQ ID NO: 77; or
h. a heavy chain variable region (VH) comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a light chain variable region (VL) comprises a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; or
i. a heavy chain variable region (VH) comprising a VH-CDR1 comprises the amino acid sequence of SEQ ID NO: 91; a VH-CDR2 comprising the amino acid sequence of SEQ ID NO: 92; a VH-CDR3 comprising the amino acid sequence of SEQ ID NO: 93; a light chain variable region (VL) comprises a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
j. a heavy chain variable region (VH) comprises a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a light chain variable region (VL) comprises a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
k. a heavy chain variable region (VH) comprises a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a light chain variable region (VL) comprises a VL-CDR1 comprising the amino acid sequence of SEQ ID NO: 115; a VL-CDR2 comprising the amino acid sequence of SEQ ID NO: 106; and a VL-CDR3 comprising the amino acid sequence of SEQ ID NO: 117; or
l. a heavy chain variable region (VH) comprises a VH-CDR1 comprising the amino acid sequence of SEQ ID NO: 281; a VH-CDR2 comprising the amino acid sequence of SEQ ID NO: 282; a VH-CDR3 comprising the amino acid sequence of SEQ ID NO: 283; a light chain variable region (VL) comprises a VL-CDR1 comprising the amino acid sequence of SEQ ID NO: 285; a VL-CDR2 comprising the amino acid sequence of SEQ ID NO: 286; and a VL-CDR3 comprising the amino acid sequence of SEQ ID NO: 287; or
m. a heavy chain variable region (VH) comprises a VH-CDR1 comprising the amino acid sequence of SEQ ID NO: 31; a VH-CDR2 comprising the amino acid sequence of SEQ ID NO: 192; a VH-CDR3 comprising the amino acid sequence of SEQ ID NO: 193; a light chain variable region (VL) comprises a VL-CDR1 comprising the amino acid sequence of SEQ ID NO: 195; a VL-CDR2 comprising the amino acid sequence of SEQ ID NO: 96; and a VL-CDR3 comprising the amino acid sequence of SEQ ID NO: 197; or
n. a heavy chain variable region (VH) comprises a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 143; a light chain variable region (VL) comprises a VL-CDR1 comprising the amino acid sequence of SEQ ID NO: 145; a VL-CDR2 comprising the amino acid sequence of SEQ ID NO: 16; and a VL-CDR3 comprising the amino acid sequence of SEQ ID NO: 17; or
o. a heavy chain variable region (VH) comprises a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a light chain variable region (VL) comprises a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
p. a heavy chain variable region (VH) comprises a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a light chain variable region (VL) comprises a VL-CDR1 comprising the amino acid sequence of SEQ ID NO: 165; a VL-CDR2 comprising the amino acid sequence of SEQ ID NO: 16; and a VL-CDR3 comprising the amino acid sequence of SEQ ID NO: 167; or
q. a heavy chain variable region (VH) comprises a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a light chain variable region (VL) comprises a VL-CDR1 comprising the amino acid sequence of SEQ ID NO: 175; a VL-CDR2 comprising the amino acid sequence of SEQ ID NO: 176; and a VL-CDR3 comprising the amino acid sequence of SEQ ID NO: 177; or
r. a heavy chain variable region (VH) comprises a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a light chain variable region (VL) comprises a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 187; or
s. a heavy chain variable region (VH) comprises a VH-CDR1 comprising the amino acid sequence of SEQ ID NO: 201; a VH-CDR2 comprising the amino acid sequence of SEQ ID NO: 202; a VH-CDR3 comprising the amino acid sequence of SEQ ID NO: 153; a light chain variable region (VL) comprises a VL-CDR1 comprising the amino acid sequence of SEQ ID NO: 105; a VL-CDR2 comprising the amino acid sequence of SEQ ID NO: 206; and a VL-CDR3 comprising the amino acid sequence of SEQ ID NO: 107; or
t. a heavy chain variable region (VH) comprises a VH-CDR1 comprising the amino acid sequence of SEQ ID NO: 211; a VH-CDR2 comprising the amino acid sequence of SEQ ID NO: 212; a VH-CDR3 comprising the amino acid sequence of SEQ ID NO: 213; a light chain variable region (VL) comprises a VL-CDR1 comprising the amino acid sequence of SEQ ID NO: 215; a VL-CDR2 comprising the amino acid sequence of SEQ ID NO: 216; and a VL-CDR3 comprising the amino acid sequence of SEQ ID NO: 217; or
u. a heavy chain variable region (VH) comprises a VH-CDR1 comprising the amino acid sequence of SEQ ID NO: 31; a VH-CDR2 comprising the amino acid sequence of SEQ ID NO: 222; a VH-CDR3 comprising the amino acid sequence of SEQ ID NO: 223; a light chain variable region (VL) comprises a VL-CDR1 comprising the amino acid sequence of SEQ ID NO: 225; a VL-CDR2 comprising the amino acid sequence of SEQ ID NO: 96; and a VL-CDR3 comprising the amino acid sequence of SEQ ID NO: 227; or
v. a heavy chain variable region (VH) comprises a VH-CDR1 comprising the amino acid sequence of SEQ ID NO: 231; a VH-CDR2 comprising the amino acid sequence of SEQ ID NO: 232; a VH-CDR3 comprising the amino acid sequence of SEQ ID NO: 233; a light chain variable region (VL) comprises a VL-CDR1 comprising the amino acid sequence of SEQ ID NO: 235; a VL-CDR2 comprising the amino acid sequence of SEQ ID NO: 236; and a VL-CDR3 comprising the amino acid sequence of SEQ ID NO: 237; or
w. a heavy chain variable region (VH) comprises a VH-CDR1 comprising the amino acid sequence of SEQ ID NO: 31; a VH-CDR2 comprising the amino acid sequence of SEQ ID NO: 242; a VH-CDR3 comprising the amino acid sequence of SEQ ID NO: 243; a light chain variable region (VL) comprises a VL-CDR1 comprising the amino acid sequence of SEQ ID NO: 225; a VL-CDR2 comprising the amino acid sequence of SEQ ID NO: 96; and a VL-CDR3 comprising the amino acid sequence of SEQ ID NO: 247; or
x. a heavy chain variable region (VH) comprises a VH-CDR1 comprising the amino acid sequence of SEQ ID NO: 31; a VH-CDR2 comprising the amino acid sequence of SEQ ID NO: 252; a VH-CDR3 comprising the amino acid sequence of SEQ ID NO: 253; a light chain variable region (VL) comprises a VL-CDR1 comprising the amino acid sequence of SEQ ID NO: 255; a VL-CDR2 comprising the amino acid sequence of SEQ ID NO: 256; and a VL-CDR3 comprising the amino acid sequence of SEQ ID NO: 257; or
y. a heavy chain variable region (VH) comprises a VH-CDR1 comprising the amino acid sequence of SEQ ID NO: 261; a VH-CDR2 comprising the amino acid sequence of SEQ ID NO: 262; a VH-CDR3 comprising the amino acid sequence of SEQ ID NO: 263; a light chain variable region (VL) comprises a VL-CDR1 comprising the amino acid sequence of SEQ ID NO: 265; a VL-CDR2 comprising the amino acid sequence of SEQ ID NO: 176; and a VL-CDR3 comprising the amino acid sequence of SEQ ID NO: 267; or
z. a heavy chain variable region (VH) comprises a VH-CDR1 comprising the amino acid sequence of SEQ ID NO: 271; a VH-CDR2 comprising the amino acid sequence of SEQ ID NO: 272; a VH-CDR3 comprising the amino acid sequence of SEQ ID NO: 273; a light chain variable region (VL) comprises a VL-CDR1 comprising the amino acid sequence of SEQ ID NO: 275; a VL-CDR2 comprising the amino acid sequence of SEQ ID NO: 276; and a VL-CDR3 comprising the amino acid sequence of SEQ ID NO: 277; or
aa. a heavy chain variable region (VH) comprises a VH-CDR1 comprising the amino acid sequence of SEQ ID NO: 301; a VH-CDR2 comprising the amino acid sequence of SEQ ID NO: 302; a VH-CDR3 comprising the amino acid sequence of SEQ ID NO: 303; a light chain variable region (VL) comprises a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 307; or
bb. a heavy chain variable region (VH) comprises a VH-CDR1 comprising the amino acid sequence of SEQ ID NO: 311; a VH-CDR2 comprising the amino acid sequence of SEQ ID NO: 312; a VH-CDR3 comprising the amino acid sequence of SEQ ID NO: 313; a light chain variable region (VL) comprises a VL-CDR1 comprising the amino acid sequence of SEQ ID NO: 315; a VL-CDR2 comprising the amino acid sequence of SEQ ID NO: 46; and a VL-CDR3 comprising the amino acid sequence of SEQ ID NO: 67; or
cc. a heavy chain variable region (VH) comprises a VH-CDR1 comprising the amino acid sequence of SEQ ID NO: 321; a VH-CDR2 comprising the amino acid sequence of SEQ ID NO: 322; a VH-CDR3 comprising the amino acid sequence of SEQ ID NO: 323; a light chain variable region (VL) comprises a VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; or
dd. a heavy chain variable region (VH) comprises a VH-CDR1 comprising the amino acid sequence of SEQ ID NO: 151; a VH-CDR2 comprising the amino acid sequence of SEQ ID NO: 332; a VH-CDR3 comprising the amino acid sequence of SEQ ID NO: 333; a light chain variable region (VL) comprises a VL-CDR1 comprising the amino acid sequence of SEQ ID NO: 335; a VL-CDR2 comprising the amino acid sequence of SEQ ID NO: 336; and a VL-CDR3 comprising the amino acid sequence of SEQ ID NO: 107; or
ee. a heavy chain variable region (VH) comprises a VH-CDR1 comprising the amino acid sequence of SEQ ID NO: 341; a VH-CDR2 comprising the amino acid sequence of SEQ ID NO: 342; a VH-CDR3 comprising the amino acid sequence of SEQ ID NO: 343; a light chain variable region (VL) comprises a VL-CDR1 comprising the amino acid sequence of SEQ ID NO: 345; a VL-CDR2 comprising the amino acid sequence of SEQ ID NO: 346; and a VL-CDR3 comprising the amino acid sequence of SEQ ID NO: 347; or
ff. a heavy chain variable region (VH) comprises a VH-CDR1 comprising the amino acid sequence of SEQ ID NO: 351; a VH-CDR2 comprising the amino acid sequence of SEQ ID NO: 352; a VH-CDR3 comprising the amino acid sequence of SEQ ID NO: 353; a light chain variable region (VL) comprises a VL-CDR1 comprising the amino acid sequence of SEQ ID NO: 355; a VL-CDR2 comprising the amino acid sequence of SEQ ID NO: 356; and a VL-CDR3 comprising the amino acid sequence of SEQ ID NO: 357; or
gg. a heavy chain variable region (VH) comprises a VH-CDR1 comprising the amino acid sequence of SEQ ID NO: 361; a VH-CDR2 comprising the amino acid sequence of SEQ ID NO: 362; a VH-CDR3 comprising the amino acid sequence of SEQ ID NO: 363; a light chain variable region (VL) comprises a VL-CDR1 comprising the amino acid sequence of SEQ ID NO: 365; a VL-CDR2 comprising the amino acid sequence of SEQ ID NO: 16; and a VL-CDR3 comprising the amino acid sequence of SEQ ID NO: 367; or
hh. a heavy chain variable region (VH) comprises a VH-CDR1 comprising the amino acid sequence of SEQ ID NO: 371; a VH-CDR2 comprising the amino acid sequence of SEQ ID NO: 372; a VH-CDR3 comprising the amino acid sequence of SEQ ID NO: 373; a light chain variable region (VL) comprises a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 347; or
ii. a heavy chain variable region (VH) comprises a VH-CDR1 comprising the amino acid sequence of SEQ ID NO: 351; a VH-CDR2 comprising the amino acid sequence of SEQ ID NO: 382; a VH-CDR3 comprising the amino acid sequence of SEQ ID NO: 383; a light chain variable region (VL) comprises a VL-CDR1 comprising the amino acid sequence of SEQ ID NO: 385; a VL-CDR2 comprising the amino acid sequence of SEQ ID NO: 386; and a VL-CDR3 comprising the amino acid sequence of SEQ ID NO: 387; or
jj. a heavy chain variable region (VH) comprises a VH-CDR1 comprising the amino acid sequence of SEQ ID NO: 351; a VH-CDR2 comprising the amino acid sequence of SEQ ID NO: 382; a VH-CDR3 comprising the amino acid sequence of SEQ ID NO: 393; a light chain variable region (VL) comprises a VL-CDR1 comprising the amino acid sequence of SEQ ID NO: 395; a VL-CDR2 comprising the amino acid sequence of SEQ ID NO: 356; and a VL-CDR3 comprising the amino acid sequence of SEQ ID NO: 357; or
kk. a heavy chain variable region (VH) comprises a VH-CDR1 comprising the amino acid sequence of SEQ ID NO: 351; a VH-CDR2 comprising the amino acid sequence of SEQ ID NO: 382; a VH-CDR3 comprising the amino acid sequence of SEQ ID NO: 393; a light chain variable region (VL) comprises a VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
ll. a heavy chain variable region (VH) comprises a VH-CDR1 comprising the amino acid sequence of SEQ ID NO: 411; a VH-CDR2 comprising the amino acid sequence of SEQ ID NO: 412; a VH-CDR3 comprising the amino acid sequence of SEQ ID NO: 413; a light chain variable region (VL) comprises a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
mm. a heavy chain variable region (VH) comprises a VH-CDR1 comprising the amino acid sequence of SEQ ID NO: 421; a VH-CDR2 comprising the amino acid sequence of SEQ ID NO: 422; a VH-CDR3 comprising the amino acid sequence GNY; a light chain variable region (VL) comprises a VL-CDR1 comprising the amino acid sequence of SEQ ID NO: 425; a VL-CDR2 comprising the amino acid sequence of SEQ ID NO: 426; and a VL-CDR3 comprising the amino acid sequence of SEQ ID NO: 427; or
nn. a heavy chain variable region (VH) comprises a VH-CDR1 comprising the amino acid sequence of SEQ ID NO: 431; a VH-CDR2 comprising the amino acid sequence of SEQ ID NO: 432; a VH-CDR3 comprising the amino acid sequence of SEQ ID NO: 433; a light chain variable region (VL) comprises a VL-CDR1 comprising the amino acid sequence of SEQ ID NO: 435; a VL-CDR2 comprising the amino acid sequence of SEQ ID NO: 436; and a VL-CDR3 comprising the amino acid sequence of SEQ ID NO: 437; or
oo. a heavy chain variable region (VH) comprises a VH-CDR1 comprising the amino acid sequence of SEQ ID NO: 151; a VH-CDR2 comprising the amino acid sequence of SEQ ID NO: 442; a VH-CDR3 comprising the amino acid sequence of SEQ ID NO: 443; a light chain variable region (VL) comprises a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
pp. a heavy chain variable region (VH) comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a light chain variable region (VL) comprises a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
qq. a heavy chain variable region (VH) comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a light chain variable region (VL) comprises a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; or
rr. a heavy chain variable region (VH) comprises a VH-CDR1 comprising the amino acid sequence of SEQ ID NO: 481; a VH-CDR2 comprising the amino acid sequence of SEQ ID NO: 482; a VH-CDR3 comprising the amino acid sequence of SEQ ID NO: 483; a light chain variable region (VL) comprises a VL-CDR1 comprising the amino acid sequence of SEQ ID NO: 165; a VL-CDR2 comprising the amino acid sequence of SEQ ID NO: 16; and a VL-CDR3 comprising the amino acid sequence of SEQ ID NO: 487; or
ss. a heavy chain variable region (VH) comprises a VH-CDR1 comprising the amino acid sequence of SEQ ID NO: 141; a VH-CDR2 comprising the amino acid sequence of SEQ ID NO: 492; a VH-CDR3 comprising the amino acid sequence of SEQ ID NO: 493; a light chain variable region (VL) comprises a VL-CDR1 comprising the amino acid sequence of SEQ ID NO: 495; a VL-CDR2 comprising the amino acid sequence of SEQ ID NO: 496; and a VL-CDR3 comprising the amino acid sequence of SEQ ID NO: 497; or
tt. a heavy chain variable region (VH) comprises a VH-CDR1 comprising the amino acid sequence of SEQ ID NO: 151; a VH-CDR2 comprising the amino acid sequence of SEQ ID NO: 502; a VH-CDR3 comprising the amino acid sequence of SEQ ID NO: 503; a light chain variable region (VL) comprises a VL-CDR1 comprising the amino acid sequence of SEQ ID NO: 105; a VL-CDR2 comprising the amino acid sequence of SEQ ID NO: 336; and a VL-CDR3 comprising the amino acid sequence of SEQ ID NO: 107; or
uu. a heavy chain variable region (VH) comprises a VH-CDR1 comprising the amino acid sequence of SEQ ID NO: 311; a VH-CDR2 comprising the amino acid sequence of SEQ ID NO: 512; a VH-CDR3 comprising the amino acid sequence of SEQ ID NO: 513; a light chain variable region (VL) comprises a VL-CDR1 comprising the amino acid sequence of SEQ ID NO: 515; a VL-CDR2 comprising the amino acid sequence of SEQ ID NO: 516; and a VL-CDR3 comprising the amino acid sequence of SEQ ID NO: 517; or
vv. a heavy chain variable region (VH) comprises a VH-CDR1 comprising the amino acid sequence of SEQ ID NO: 521; a VH-CDR2 comprising the amino acid sequence of SEQ ID NO: 522; a VH-CDR3 comprising the amino acid sequence of SEQ ID NO: 463; a light chain variable region (VL) comprises a VL-CDR1 comprising the amino acid sequence of SEQ ID NO: 525; a VL-CDR2 comprising the amino acid sequence of SEQ ID NO: 16; and a VL-CDR3 comprising the amino acid sequence of SEQ ID NO: 467; or
ww. a heavy chain variable region (VH) comprises a VH-CDR1 comprising the amino acid sequence of SEQ ID NO: 371; a VH-CDR2 comprising the amino acid sequence of SEQ ID NO: 532; a VH-CDR3 comprising the amino acid sequence of SEQ ID NO: 533; a light chain variable region (VL) comprises a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; or
xx. a heavy chain variable region (VH) comprises a VH-CDR1 comprising the amino acid sequence of SEQ ID NO: 341; a VH-CDR2 comprising the amino acid sequence of SEQ ID NO: 542; a VH-CDR3 comprising the amino acid sequence of SEQ ID NO: 543; a light chain variable region (VL) comprises a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 347; or
yy. a heavy chain variable region (VH) comprises a VH-CDR1 comprising the amino acid sequence of SEQ ID NO: 551; a VH-CDR2 comprising the amino acid sequence of SEQ ID NO: 552; a VH-CDR3 comprising the amino acid sequence of SEQ ID NO: 553; a light chain variable region (VL) comprises a VL-CDR1 comprising the amino acid sequence of SEQ ID NO: 555; a VL-CDR2 comprising the amino acid sequence of SEQ ID NO: 16; and a VL-CDR3 comprising the amino acid sequence of SEQ ID NO: 557; or
zz. a heavy chain variable region (VH) comprises a VH-CDR1 comprising the amino acid sequence of SEQ ID NO: 551; a VH-CDR2 comprising the amino acid sequence of SEQ ID NO: 552; a VH-CDR3 comprising the amino acid sequence of SEQ ID NO: 563; a light chain variable region (VL) comprises a VL-CDR1 comprising the amino acid sequence of SEQ ID NO: 555; a VL-CDR2 comprising the amino acid sequence of SEQ ID NO: 16; and a VL-CDR3 comprising the amino acid sequence of SEQ ID NO: 557; or
aaa. a heavy chain variable region (VH) comprises a VH-CDR1 comprising the amino acid sequence of SEQ ID NO: 571; a VH-CDR2 comprising the amino acid sequence of SEQ ID NO: 202; a VH-CDR3 comprising the amino acid sequence of SEQ ID NO: 573; a light chain variable region (VL) comprises a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
bbb. a heavy chain variable region (VH) comprises a VH-CDR1 comprising the amino acid sequence of SEQ ID NO: 581; a VH-CDR2 comprising the amino acid sequence of SEQ ID NO: 582; a VH-CDR3 comprising the amino acid sequence of SEQ ID NO: 583; a light chain variable region (VL) comprises a VL-CDR1 comprising the amino acid sequence of SEQ ID NO: 585; a VL-CDR2 comprising the amino acid sequence of SEQ ID NO: 586; and a VL-CDR3 comprising the amino acid sequence of SEQ ID NO: 587; or
ccc. a heavy chain variable region (VH) comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a light chain variable region (VL) comprises a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; or
ddd. a heavy chain variable region (VH) comprises a VH-CDR1 comprising the amino acid sequence of SEQ ID NO: 611; a VH-CDR2 comprising the amino acid sequence of SEQ ID NO: 612; a VH-CDR3 comprising the amino acid sequence of SEQ ID NO: 613; a light chain variable region (VL) comprises a VL-CDR1 comprising the amino acid sequence of SEQ ID NO: 615; a VL-CDR2 comprising the amino acid sequence of SEQ ID NO: 616; and a VL-CDR3 comprising the amino acid sequence of SEQ ID NO: 617; or
eee. a heavy chain variable region (VH) comprises a VH-CDR1 comprising the amino acid sequence of SEQ ID NO: 621; a VH-CDR2 comprising the amino acid sequence of SEQ ID NO: 622; a VH-CDR3 comprising the amino acid sequence of SEQ ID NO: 623; a light chain variable region (VL) comprises a VL-CDR1 comprising the amino acid sequence of SEQ ID NO: 625; a VL-CDR2 comprising the amino acid sequence of SEQ ID NO: 626; and a VL-CDR3 comprising the amino acid sequence of SEQ ID NO: 627; or
fff. a heavy chain variable region (VH) comprises a VH-CDR1 comprising the amino acid sequence of SEQ ID NO: 631; a VH-CDR2 comprising the amino acid sequence of SEQ ID NO: 632; a VH-CDR3 comprising the amino acid sequence of SEQ ID NO: 633; a light chain variable region (VL) comprises a VL-CDR1 comprising the amino acid sequence of SEQ ID NO: 635; a VL-CDR2 comprising the amino acid sequence of SEQ ID NO: 616; and a VL-CDR3 comprising the amino acid sequence of SEQ ID NO: 637; or
ggg. a heavy chain variable region (VH) comprises a VH-CDR1 comprising the amino acid sequence of SEQ ID NO: 641; a VH-CDR2 comprising the amino acid sequence of SEQ ID NO: 642; a VH-CDR3 comprising the amino acid sequence of SEQ ID NO: 643; a light chain variable region (VL) comprises a VL-CDR1 comprising the amino acid sequence of SEQ ID NO: 625; a VL-CDR2 comprising the amino acid sequence of SEQ ID NO: 626; and a VL-CDR3 comprising the amino acid sequence of SEQ ID NO: 627; or
hhh. a heavy chain variable region (VH) comprises a VH-CDR1 comprising the amino acid sequence of SEQ ID NO: 621; a VH-CDR2 comprising the amino acid sequence of SEQ ID NO: 642; a VH-CDR3 comprising the amino acid sequence of SEQ ID NO: 653; a light chain variable region (VL) comprises a VL-CDR1 comprising the amino acid sequence of SEQ ID NO: 655; a VL-CDR2 comprising the amino acid sequence of SEQ ID NO: 626; and a VL-CDR3 comprising the amino acid sequence of SEQ ID NO: 627; or
iii. a heavy chain variable region (VH) comprises a VH-CDR1 comprising the amino acid sequence of SEQ ID NO: 661; a VH-CDR2 comprising the amino acid sequence of SEQ ID NO: 662; a VH-CDR3 comprising the amino acid sequence of SEQ ID NO: 663; a light chain variable region (VL) comprises a VL-CDR1 comprising the amino acid sequence of SEQ ID NO: 665; a VL-CDR2 comprising the amino acid sequence of SEQ ID NO: 666; and a VL-CDR3 comprising the amino acid sequence of SEQ ID NO: 667; or
jjj. a heavy chain variable region (VH) comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a light chain variable region (VL) comprises a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; or
kkk. a heavy chain variable region (VH) comprises a VH-CDR1 comprising the amino acid sequence of SEQ ID NO: 621; a VH-CDR2 comprising the amino acid sequence of SEQ ID NO: 642; a VH-CDR3 comprising the amino acid sequence of SEQ ID NO: 683; a light chain variable region (VL) comprises a VL-CDR1 comprising the amino acid sequence of SEQ ID NO: 625; a VL-CDR2 comprising the amino acid sequence of SEQ ID NO: 686; and a VL-CDR3 comprising the amino acid sequence of SEQ ID NO: 627; or
lll. a heavy chain variable region (VH) comprises a VH-CDR1 comprising the amino acid sequence of SEQ ID NO: 691; a VH-CDR2 comprising the amino acid sequence of SEQ ID NO: 692; a VH-CDR3 comprising the amino acid sequence of SEQ ID NO: 693; a light chain variable region (VL) comprises a VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
mmm. a heavy chain variable region (VH) comprises a VH-CDR1 comprising the amino acid sequence of SEQ ID NO: 701; a VH-CDR2 comprising the amino acid sequence of SEQ ID NO: 702; a VH-CDR3 comprising the amino acid sequence of SEQ ID NO: 703; a light chain variable region (VL) comprises a VL-CDR1 comprising the amino acid sequence of SEQ ID NO: 705; a VL-CDR2 comprising the amino acid sequence of SEQ ID NO: 706; and a VL-CDR3 comprising the amino acid sequence of SEQ ID NO: 707; or
nnn. a heavy chain variable region (VH) comprises a VH-CDR1 comprising the amino acid sequence of SEQ ID NO: 711; a VH-CDR2 comprising the amino acid sequence of SEQ ID NO: 712; a VH-CDR3 comprising the amino acid sequence of SEQ ID NO: 713; a light chain variable region (VL) comprises a VL-CDR1 comprising the amino acid sequence of SEQ ID NO: 715; a VL-CDR2 comprising the amino acid sequence of SEQ ID NO: 716; and a VL-CDR3 comprising the amino acid sequence of SEQ ID NO: 717; or
ooo. a heavy chain variable region (VH) comprises a VH-CDR1 comprising the amino acid sequence of SEQ ID NO: 721; a VH-CDR2 comprising the amino acid sequence of SEQ ID NO: 722; a VH-CDR3 comprising the amino acid sequence of SEQ ID NO: 723; a light chain variable region (VL) comprises a VL-CDR1 comprising the amino acid sequence of SEQ ID NO: 725; a VL-CDR2 comprising the amino acid sequence of SEQ ID NO: 616; and a VL-CDR3 comprising the amino acid sequence of SEQ ID NO: 637; or
ppp. a heavy chain variable region (VH) comprises a VH-CDR1 comprising the amino acid sequence of SEQ ID NO: 731; a VH-CDR2 comprising the amino acid sequence of SEQ ID NO: 722; a VH-CDR3 comprising the amino acid sequence of SEQ ID NO: 733; a light chain variable region (VL) comprises a VL-CDR1 comprising the amino acid sequence of SEQ ID NO: 735; a VL-CDR2 comprising the amino acid sequence of SEQ ID NO: 736; and a VL-CDR3 comprising the amino acid sequence of SEQ ID NO: 637; or
qqq. a heavy chain variable region (VH) comprises a VH-CDR1 comprising the amino acid sequence of SEQ ID NO: 671; a VH-CDR2 comprising the amino acid sequence of SEQ ID NO: 742; a VH-CDR3 comprising the amino acid sequence of SEQ ID NO: 743; a light chain variable region (VL) comprises a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; or
rrr. a heavy chain variable region (VH) comprises a VH-CDR1 comprising the amino acid sequence of SEQ ID NO: 761; a VH-CDR2 comprising the amino acid sequence of SEQ ID NO: 722; a VH-CDR3 comprising the amino acid sequence of SEQ ID NO: 733; a light chain variable region (VL) comprises a VL-CDR1 comprising the amino acid sequence of SEQ ID NO: 765; a VL-CDR2 comprising the amino acid sequence of SEQ ID NO: 616; and a VL-CDR3 comprising the amino acid sequence of SEQ ID NO: 637; or
sss. a heavy chain variable region (VH) comprises a VH-CDR1 comprising the amino acid sequence of SEQ ID NO: 771; a VH-CDR2 comprising the amino acid sequence of SEQ ID NO: 772; a VH-CDR3 comprising the amino acid sequence of SEQ ID NO: 773; a light chain variable region (VL) comprises a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; or
ttt. a heavy chain variable region (VH) comprises a VH-CDR1 comprising the amino acid sequence of SEQ ID NO: 751; a VH-CDR2 comprising the amino acid sequence of SEQ ID NO: 722; a VH-CDR3 comprising the amino acid sequence of SEQ ID NO: 723; a light chain variable region (VL) comprises a VL-CDR1 comprising the amino acid sequence of SEQ ID NO: 735; a VL-CDR2 comprising the amino acid sequence of SEQ ID NO: 616; and a VL-CDR3 comprising the amino acid sequence of SEQ ID NO: 637; or
uuu. a heavy chain variable region (VH) comprises a VH-CDR1 comprising the amino acid sequence of SEQ ID NO: 791; a VH-CDR2 comprising the amino acid sequence of SEQ ID NO: 792; a VH-CDR3 comprising the amino acid sequence of SEQ ID NO: 793; a light chain variable region (VL) comprises a VL-CDR1 comprising the amino acid sequence of SEQ ID NO: 795; a VL-CDR2 comprising the amino acid sequence of SEQ ID NO: 616; and a VL-CDR3 comprising the amino acid sequence of SEQ ID NO: 797; or
vvv. a heavy chain variable region (VH) comprises a VH-CDR1 comprising the amino acid sequence of SEQ ID NO: 771; a VH-CDR2 comprising the amino acid sequence of SEQ ID NO: 802; a VH-CDR3 comprising the amino acid sequence of SEQ ID NO: 803; a light chain variable region (VL) comprises a VL-CDR1 comprising the amino acid sequence of SEQ ID NO: 805; a VL-CDR2 comprising the amino acid sequence of SEQ ID NO: 676; and a VL-CDR3 comprising the amino acid sequence of SEQ ID NO: 677; or
www. a heavy chain variable region (VH) comprises a VH-CDR1 comprising the amino acid sequence of SEQ ID NO: 811; a VH-CDR2 comprising the amino acid sequence of SEQ ID NO: 812; a VH-CDR3 comprising the amino acid sequence of SEQ ID NO: 813; a light chain variable region (VL) comprises a VL-CDR1 comprising the amino acid sequence of SEQ ID NO: 815; a VL-CDR2 comprising the amino acid sequence of SEQ ID NO: 696; and a VL-CDR3 comprising the amino acid sequence of SEQ ID NO: 817; or
xxx. a heavy chain variable region (VH) comprises a VH-CDR1 comprising the amino acid sequence of SEQ ID NO: 821; a VH-CDR2 comprising the amino acid sequence of SEQ ID NO: 822; a VH-CDR3 comprising the amino acid sequence of SEQ ID NO: 823; a light chain variable region (VL) comprises a VL-CDR1 comprising the amino acid sequence of SEQ ID NO: 825; a VL-CDR2 comprising the amino acid sequence of SEQ ID NO: 826; and a VL-CDR3 comprising the amino acid sequence of SEQ ID NO: 827; or
yyy. a heavy chain variable region (VH) comprises a VH-CDR1 comprising the amino acid sequence of SEQ ID NO: 831; a VH-CDR2 comprising the amino acid sequence of SEQ ID NO: 832; a VH-CDR3 comprising the amino acid sequence of SEQ ID NO: 833; a light chain variable region (VL) comprises a VL-CDR1 comprising the amino acid sequence of SEQ ID NO: 835; a VL-CDR2 comprising the amino acid sequence of SEQ ID NO: 836; and a VL-CDR3 comprising the amino acid sequence of SEQ ID NO: 817; or
zzz. a heavy chain variable region (VH) comprises a VH-CDR1 comprising the amino acid sequence of SEQ ID NO: 841; a VH-CDR2 comprising the amino acid sequence of SEQ ID NO: 842; a VH-CDR3 comprising the amino acid sequence of SEQ ID NO: 843; a light chain variable region (VL) comprises a VL-CDR1 comprising the amino acid sequence of SEQ ID NO: 845; a VL-CDR2 comprising the amino acid sequence of SEQ ID NO: 846; and a VL-CDR3 comprising the amino acid sequence of SEQ ID NO: 847.

77. The alpha-synuclein biparatopic antibody or functional fragment thereof, or a mixture comprising at least two monospecific antibodies or functional fragments thereof, according to claim 65, comprising two distinct pairs of variable regions VH and VL, wherein:
a. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second distinct pair of variable regions VH and VL comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; or
b. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; or
c. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 91; a VH-CDR2 comprising the amino acid sequence of SEQ ID NO: 92; and a VH-CDR3 comprising the amino acid sequence of SEQ ID NO: 93; VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
d. a first pair of variable regions VH and VL comprises a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second pair of variable regions VH and VL comprises a VH-CDR1 comprising the amino acid sequence of SEQ ID NO: 31; VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; or
e. a first pair of variable regions VH and VL comprises a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second pair of variable regions VH and VL comprises a VH-CDR1 comprising the amino acid sequence of SEQ ID NO: 91; a VH-CDR2 comprising the amino acid sequence of SEQ ID NO: 92; and a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
f. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
g. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises comprising a VH-CDR1 comprising the amino acid sequence of SEQ ID NO: 181; a VH-CDR2 comprising the amino acid sequence of SEQ ID NO: 182; and a VH-CDR3 comprising the amino acid sequence of SEQ ID NO: 183; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 187; or
h. a first pair of variable regions VH and VL comprises a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i. a first pair of variable regions VH and VL comprises a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; and a second pair of variable regions VH and VL comprises a VH-CDR1 comprising the amino acid sequence of SEQ ID NO: 181; a VH-CDR2 comprising the amino acid sequence of SEQ ID NO: 182; and a VH-CDR3 comprising the amino acid sequence of SEQ ID NO: 183; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 187; or
j. a first pair of variable regions VH and VL comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
k. a first pair of variable regions VH and VL comprises a VH-CDR1 comprising the amino acid sequence of SEQ ID NO: 691; a VH-CDR2 comprising the amino acid sequence of SEQ ID NO: 692; a VH-CDR3 comprising the amino acid sequence of SEQ ID NO: 693; a VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; and a second pair of variable regions VH and VL comprises a VH-CDR1 comprising the amino acid sequence of SEQ ID NO: 671; a VH-CDR2 comprising the amino acid sequence of SEQ ID NO: 672; and a VH-CDR3 comprising the amino acid sequence of SEQ ID NO: 673; VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; or
l. a first pair of variable regions VH and VL comprises a VH-CDR1 comprising the amino acid sequence of SEQ ID NO: 321; a VH-CDR2 comprising the amino acid sequence of SEQ ID NO: 322; a VH-CDR3 comprising the amino acid sequence of SEQ ID NO: 323; a VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
m. a first pair of variable regions VH and VL comprises a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second pair of variable regions VH and VL comprises a VH-CDR1 comprising the amino acid sequence of SEQ ID NO: 361; a VH-CDR2 comprising the amino acid sequence of SEQ ID NO: 362; and a VH-CDR3 comprising the amino acid sequence of SEQ ID NO: 363; VL-CDR1 comprising the amino acid sequence of SEQ ID NO: 365; a VL-CDR2 comprising the amino acid sequence of SEQ ID NO: 16; and a VL-CDR3 comprising the amino acid sequence of SEQ ID NO: 367; or
n. a first pair of variable regions VH and VL comprises a VH-CDR1 comprising the amino acid sequence of SEQ ID NO: 351; a VH-CDR2 comprising the amino acid sequence of SEQ ID NO: 382; a VH-CDR3 comprising the amino acid sequence of SEQ ID NO: 393; a VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; and a second pair of variable regions VH and VL comprises a VH-CDR1 comprising the amino acid sequence of SEQ ID NO: 91; a VH-CDR2 comprising the amino acid sequence of SEQ ID NO: 92; and a VH-CDR3 comprising the amino acid sequence of SEQ ID NO: 93; VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
o. a first pair of variable regions VH and VL comprises a VH-CDR1 comprising the amino acid sequence of SEQ ID NO: 371; a VH-CDR2 comprising the amino acid sequence of SEQ ID NO: 532; a VH-CDR3 comprising the amino acid sequence of SEQ ID NO: 533; a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
p. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 371; a VH-CDR2 comprising the amino acid sequence of SEQ ID NO: 532; and a VH-CDR3 comprising the amino acid sequence of SEQ ID NO: 533; VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; or
q. a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
r. a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
s. a first pair of variable regions VH and VL comprises a VH-CDR1 comprising the amino acid sequence of SEQ ID NO: 371; a VH-CDR2 comprising the amino acid sequence of SEQ ID NO: 532; a VH-CDR3 comprising the amino acid sequence of SEQ ID NO: 533; a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
t. a first pair of variable regions VH and VL comprises a VH-CDR1 comprising the amino acid sequence of SEQ ID NO: 321; a VH-CDR2 comprising the amino acid sequence of SEQ ID NO: 322; a VH-CDR3 comprising the amino acid sequence of SEQ ID NO: 323; a VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
u. a first pair of variable regions VH and VL comprises a VH-CDR1 comprising the amino acid sequence of SEQ ID NO: 21; a VH-CDR2 comprising the amino acid sequence of SEQ ID NO: 52; a VH-CDR3 comprising the amino acid sequence YSF; a VL-CDR1 comprising the amino acid sequence of SEQ ID NO: 55; a VL-CDR2 comprising the amino acid sequence of SEQ ID NO: 56; and a VL-CDR3 comprising the amino acid sequence of SEQ ID NO: 27; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
v. a first pair of variable regions VH and VL comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
w. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
x. a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
y. a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
z. a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 321; a VH-CDR2 comprising the amino acid sequence of SEQ ID NO: 322; and a VH-CDR3 comprising the amino acid sequence of SEQ ID NO: 323; VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; or
aa. a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 571; a VH-CDR2 comprising the amino acid sequence of SEQ ID NO: 202; and a VH-CDR3 comprising the amino acid sequence of SEQ ID NO: 573; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
bb. a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 341; a VH-CDR2 comprising the amino acid sequence of SEQ ID NO: 342; and a VH-CDR3 comprising the amino acid sequence of SEQ ID NO: 343; VL-CDR1 comprising the amino acid sequence of SEQ ID NO: 345; a VL-CDR2 comprising the amino acid sequence of SEQ ID NO: 346; and a VL-CDR3 comprising the amino acid sequence of SEQ ID NO: 347; or
cc. a first pair of variable regions VH and VL comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; and a second pair of variable regions VH and VL comprises a VH-CDR1 comprising the amino acid sequence of SEQ ID NO: 141; a VH-CDR2 comprising the amino acid sequence of SEQ ID NO: 472; and a VH-CDR3 comprising the amino acid sequence of SEQ ID NO: 473; VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; or
dd. a first pair of variable regions VH and VL comprises a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 85; a VL-CDR2 comprising the amino acid sequence of SEQ ID NO: 36; and a VL-CDR3 comprising the amino acid sequence of SEQ ID NO: 87; and a second pair of variable regions VH and VL comprises a VH-CDR1 comprising the amino acid sequence of SEQ ID NO: 311; a VH-CDR2 comprising the amino acid sequence of SEQ ID NO: 512; and a VH-CDR3 comprising the amino acid sequence of SEQ ID NO: 513; VL-CDR1 comprising the amino acid sequence of SEQ ID NO: 515; a VL-CDR2 comprising the amino acid sequence of SEQ ID NO: 516; and a VL-CDR3 comprising the amino acid sequence of SEQ ID NO: 517; or
ee. a first pair of variable regions VH and VL comprises a VH-CDR1 comprising the amino acid sequence of SEQ ID NO: 341; a VH-CDR2 comprising the amino acid sequence of SEQ ID NO: 342; a VH-CDR3 comprising the amino acid sequence of SEQ ID NO: 343; a VL-CDR1 comprising the amino acid sequence of SEQ ID NO: 345; a VL-CDR2 comprising the amino acid sequence of SEQ ID NO: 346; and a VL-CDR3 comprising the amino acid sequence of SEQ ID NO: 347; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
ff. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
gg. a first pair of variable regions VH and VL comprises a VH-CDR1 comprising the amino acid sequence of SEQ ID NO: 691; a VH-CDR2 comprising the amino acid sequence of SEQ ID NO: 692; a VH-CDR3 comprising the amino acid sequence of SEQ ID NO: 693; a VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
hh. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 151; a VH-CDR2 comprising the amino acid sequence of SEQ ID NO: 332; and a VH-CDR3 comprising the amino acid sequence of SEQ ID NO: 333; VL-CDR1 comprising the amino acid sequence of SEQ ID NO: 335; a VL-CDR2 comprising the amino acid sequence of SEQ ID NO: 336; and a VL-CDR3 comprising the amino acid sequence of SEQ ID NO: 107; or
ii. a first pair of variable regions VH and VL comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
jj. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 611; a VH-CDR2 comprising the amino acid sequence of SEQ ID NO: 612; and a VH-CDR3 comprising the amino acid sequence of SEQ ID NO: 613; VL-CDR1 comprising the amino acid sequence of SEQ ID NO: 615; a VL-CDR2 comprising the amino acid sequence of SEQ ID NO: 616; and a VL-CDR3 comprising the amino acid sequence of SEQ ID NO: 617.

78. The alpha-synuclein biparatopic antibody or functional fragment thereof, or a mixture comprising at least two monospecific antibodies or functional fragments thereof, according to claim 65, comprising two distinct pairs of variable regions VH and VL, wherein:
a. a first pair of variable regions VH and VL comprises a VH-CDR1 comprising the amino acid sequence of SEQ ID NO: 321; a VH-CDR2 comprising the amino acid sequence of SEQ ID NO: 322; a VH-CDR3 comprising the amino acid sequence of SEQ ID NO: 323; a VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
b. a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
c. a first pair of variable regions VH and VL comprises a VH-CDR1 comprising the amino acid sequence of SEQ ID NO: 371; a VH-CDR2 comprising the amino acid sequence of SEQ ID NO: 532; a VH-CDR3 comprising the amino acid sequence of SEQ ID NO: 533; a VL-CDR1 comprising the amino acid sequence of SEQ ID NO: 345; a VL-CDR2 comprising the amino acid sequence of SEQ ID NO: 376; and a VL-CDR3 comprising the amino acid sequence of SEQ ID NO: 537; and a second pair of variable regions VH and VL comprises a VH-CDR1 comprising the amino acid sequence of SEQ ID NO: 351; a VH-CDR2 comprising the amino acid sequence of SEQ ID NO: 382; and a VH-CDR3 comprising the amino acid sequence of SEQ ID NO: 393; VL-CDR1 comprising the amino acid sequence of SEQ ID NO: 405; a VL-CDR2 comprising the amino acid sequence of SEQ ID NO: 356; and a VL-CDR3 comprising the amino acid sequence of SEQ ID NO: 357; or
d. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 461; a VH-CDR2 comprising the amino acid sequence of SEQ ID NO: 462; and a VH-CDR3 comprising the amino acid sequence of SEQ ID NO: 463; VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; or
e. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
f. a first pair of variable regions VH and VL comprises a VH-CDR1 comprising the amino acid sequence of SEQ ID NO: 141; a VH-CDR2 comprising the amino acid sequence of SEQ ID NO: 472; a VH-CDR3 comprising the amino acid sequence of SEQ ID NO: 473; a VL-CDR1 comprising the amino acid sequence of SEQ ID NO: 475; a VL-CDR2 comprising the amino acid sequence of SEQ ID NO: 476; and a VL-CDR3 comprising the amino acid sequence of SEQ ID NO: 477; and a second pair of variable regions VH and VL comprises a VH-CDR1 comprising the amino acid sequence of SEQ ID NO: 321; a VH-CDR2 comprising the amino acid sequence of SEQ ID NO: 322; and a VH-CDR3 comprising the amino acid sequence of SEQ ID NO: 323; VL-CDR1 comprising the amino acid sequence of SEQ ID NO: 325; a VL-CDR2 comprising the amino acid sequence of SEQ ID NO: 326; and a VL-CDR3 comprising the amino acid sequence of SEQ ID NO: 327; or
g. a first pair of variable regions VH and VL comprises a VH-CDR1 comprising the amino acid sequence of SEQ ID NO: 691; a VH-CDR2 comprising the amino acid sequence of SEQ ID NO: 692; a VH-CDR3 comprising the amino acid sequence of SEQ ID NO: 693; a VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
h. a first pair of variable regions VH and VL comprises a VH-CDR1 comprising the amino acid sequence of SEQ ID NO: 671; a VH-CDR2 comprising the amino acid sequence of SEQ ID NO: 672; a VH-CDR3 comprising the amino acid sequence of SEQ ID NO: 673; a VL-CDR1 comprising the amino acid sequence of SEQ ID NO: 675; a VL-CDR2 comprising the amino acid sequence of SEQ ID NO: 676; and a VL-CDR3 comprising the amino acid sequence of SEQ ID NO: 677; and a second pair of variable regions VH and VL comprises a VH-CDR1 comprising the amino acid sequence of SEQ ID NO: 11; a VH-CDR2 comprising the amino acid sequence of SEQ ID NO: 12; and a VH-CDR3 comprising the amino acid sequence of SEQ ID NO: 13; VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17.

79. An immunoconjugate comprising the alpha-synuclein biparatopic antibody or functional fragment thereof according to claim 65, or comprising the mixture comprising at least two monospecific antibodies or functional fragments thereof according to claim 65.

80. An immunoconjugate according to claim 79, wherein the immunoconjugate crosses the blood brain barrier using a delivery vehicle or a blood brain barrier moiety; optionally wherein the delivery vehicle comprises a liposome or extracellular vesicle or
wherein the alpha-synuclein biparatopic antibody or functional fragment thereof is, or at least two monospecific antibodies or functional fragments thereof are, linked to a blood brain barrier moiety;
optionally wherein the blood brain barrier moiety is a polypeptide or a small molecule, preferably, a peptide, a receptor ligand, a single domain antibody (VHH), a scFv or a Fab fragment;
optionally wherein the blood brain barrier moiety binds a blood brain barrier receptor
optionally wherein the blood brain barrier receptor comprises a receptor transfer unit, a transferrin receptor, insulin receptor or low-density lipoprotein receptor.

81. A method for the diagnosis, prevention, alleviation and/or treatment of a disease, disorder or abnormality associated with alpha-synuclein aggregates or pathological alpha-synuclein, the method comprising administering an alpha-synuclein biparatopic antibody or functional fragment thereof, or the mixture comprising at least two monospecific antibodies or functional fragments thereof, according to claim 65, or the immunoconjugate of claim 79.

82. The alpha-synuclein biparatopic antibody or functional fragment thereof, or the mixture comprising at least two monospecific antibodies or functional fragments thereof, according to claim 65, or the immunoconjugate of claim 79 for use as an analytical reference or an in vitro screening tool, or in diagnosis.

83. The method of claim 81, wherein the aggregates are Lewy bodies, Lewy neurites and/or glial cytoplasmic inclusions, and/or
the disease, disorder or abnormality is a synucleinopathy;
optionally wherein the disease, disorder or abnormality associated with alpha-synuclein aggregates or pathological alpha-synuclein is selected from the group consisting of Parkinson's disease (sporadic, familial with alpha-synuclein mutations, familial with mutations other than alpha-synuclein, pure autonomic failure and Lewy body dysphagia), Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

84. A nucleic acid encoding the alpha-synuclein biparatopic antibody or functional fragments thereof, or the mixture comprising at least two monospecific antibodies or functional fragments thereof of claim 65, optionally wherein the nucleic acid is a part of a viral vector for targeted delivery to the blood brain barrier or any other cell type in the CNS; optionally wherein the viral vector is a recombinant adeno-associated viral vector (rAAV), preferably a recombinant adeno-associated viral vector selected from AAV1 to AAV12.

85. A recombinant expression vector comprising the nucleic acid of claim 84.

86. A host cell comprising the nucleic acid of claim 84, optionally wherein the nucleic acid is comprised within a recombinant expression vector.

87. A cell-free expression system containing the recombinant expression vector of claim 85.

88. A method of producing alpha-synuclein biparatopic antibody or functional fragments thereof, or a mixture comprising at least two monospecific antibodies or functional fragments thereof, according to claim 65, comprising the steps of:
a. culturing the host cell of claim 86 or the cell-free expression system of claim 87 under conditions suitable for producing alpha-synuclein biparatopic antibody or functional fragments thereof, or the mixture comprising at least two monospecific antibodies or functional fragments thereof, and
b. isolating alpha-synuclein biparatopic antibody or functional fragments thereof, or the mixture comprising at least two monospecific antibodies or functional fragments thereof.

89. A pharmaceutical composition comprising the alpha-synuclein biparatopic antibody or functional fragments thereof, or the mixture comprising at least two monospecific antibodies or functional fragments thereof, according to claim 65, or the immunoconjugate of claim 79, and a pharmaceutically acceptable carrier.

90. The method of claim 81, wherein the disease, disorder or abnormality associated with alpha-synuclein aggregates is Parkinson's disease or multiple system atrophy.

91. The method of claim 81, which comprises administering at least one additional therapeutic agent.

92. The alpha-synuclein biparatopic antibody or functional fragment thereof, or a mixture comprising at least two monospecific antibodies or functional fragments thereof, according to claim 65, comprising a first binding site which binds a first epitope and a second distinct binding site which binds to a second distinct epitope, wherein:
a. the first epitope is situated within amino acid residues 82-96 (SEQ ID NO: 130) of human alpha-synuclein of SEQ ID NO: 1 and the second epitope is situated within amino acid residues 124-131 (SEQ ID NO: 7) of human alpha-synuclein of SEQ ID NO: 1; or
b. the first and second epitope are situated within amino acid residues 100-114 (SEQ ID NO: 132) of human alpha-synuclein of SEQ ID NO: 1;
optionally wherein:
c. the first epitope is situated within amino acid residues 82-96 (SEQ ID NO: 130) of human alpha-synuclein of SEQ ID NO: 1 and critical amino acid residues for binding comprise, or consist of, amino acid residues 92-94 and 96 and the second epitope is situated within amino acid residues 124-131 (SEQ ID NO: 7) of human alpha-synuclein of SEQ ID NO: 1 and critical amino acid residues for binding comprise, or consist of, amino acid residues 126-127; or
d. the first and second epitope are situated within amino acid residues 100-114 (SEQ ID NO: 132) of human alpha-synuclein of SEQ ID NO: 1 and critical amino acid residues for binding within the first epitope comprise, or consist of, amino acid residues 100-105.

93. The alpha-synuclein biparatopic antibody or functional fragment thereof, or a mixture comprising at least two monospecific antibodies or functional fragments thereof, according to claim 65, comprising two distinct pairs of variable regions VH and VL, wherein:
a. a first pair of variable regions VH and VL comprising a VH which has at least 94%, 95%, 96%, 97%, 98%, 99% or 100% sequence identity to the amino acid sequence of SEQ ID NO: 460; and a VL which has at least 99% or 100% sequence identity to the amino acid sequence of SEQ ID NO: 464; and a second pair of variable regions VH and VL comprising a VH which has at least 87%, 88%, 89%, 90%, 91%, 92%, 93%, 94%, 95%, 96%, 97%, 98%, 99% or 100% sequence identity to the amino acid sequence of SEQ ID NO: 690; and a VL which has at least 96%, 97%, 98%, 99% or 100% sequence identity to the amino acid sequence of SEQ ID NO: 694; or
b. a first pair of variable regions VH and VL comprising a VH which has at least 90%, 91%, 92%, 93%, 94%, 95%, 96%, 97%, 98%, 99% or 100% sequence identity to the amino acid sequence of SEQ ID NO: 150; and a VL has at least 98%, 99% or 100% sequence identity to the amino acid sequence of SEQ ID NO: 154; and a second pair of variable regions VH and VL comprising a VH which has at least 96%, 97%, 98%, 99% or 100% sequence identity to the amino acid sequence of SEQ ID NO: 10; and a VL which has at least 99% or 100% sequence identity to the amino acid sequence of SEQ ID NO: 14;
optionally wherein:
c. a first pair of variable regions VH and VL comprises a VH and VL respectively, set forth in SEQ ID NO: 460 and SEQ ID NO: 464; and a second pair of variable regions VH and VL comprises a VH and VL respectively, set forth in SEQ ID NO: 690 and SEQ ID NO: 694; or
d. a first pair of variable regions VH and VL comprises a VH and VL respectively, set forth in SEQ ID NO: 150 and SEQ ID NO: 154; and a second pair of variable regions VH and VL comprises a VH and VL respectively, set forth in SEQ ID NO: 10 and SEQ ID NO: 14;
optionally wherein:
e. a first pair of variable regions VH and VL comprises a VH-CDR1 comprising the amino acid sequence of SEQ ID NO: 461; a VH-CDR2 comprising the amino acid sequence of SEQ ID NO: 462; a VH-CDR3 comprising the amino acid sequence of SEQ ID NO: 463; a VL-CDR1 comprising the amino acid sequence of SEQ ID NO: 465; a VL-CDR2 comprising the amino acid sequence of SEQ ID NO: 16; and a VL-CDR3 comprising the amino acid sequence of SEQ ID NO: 467; and a second pair of variable regions VH and VL comprises a VH-CDR1 comprising the amino acid sequence of SEQ ID NO: 691; a VH-CDR2 comprising the amino acid sequence of SEQ ID NO: 692; and a VH-CDR3 comprising the amino acid sequence of SEQ ID NO: 693; VL-CDR1 comprising the amino acid sequence of SEQ ID NO: 695; a VL-CDR2 comprising the amino acid sequence of SEQ ID NO: 696; and a VL-CDR3 comprising the amino acid sequence of SEQ ID NO: 697; or
f. a first pair of variable regions VH and VL comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and a second pair of variable regions VH and VL comprises a VH-CDR1 comprising the amino acid sequence of SEQ ID NO: 151; a VH-CDR2 comprising the amino acid sequence of SEQ ID NO: 152; and a VH-CDR3 comprising the amino acid sequence of SEQ ID NO: 153; VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107.

94. A method for monitoring a disease, disorder and/or condition associated with alpha-synuclein at two or more time points using samples from a subject comprising contacting the samples with an alpha-synuclein biparatopic antibody or functional fragment thereof, or a mixture comprising at least two monospecific antibodies or functional fragments thereof, according to claim 65, wherein:
a. higher levels of alpha-synuclein in the later sample compared with one or more earlier samples are indicative of progression of a disease, disorder and/or condition associated with alpha-synuclein;
b. lower levels of alpha-synuclein in the later sample compared with one or more earlier samples are indicative of regression of a disease, disorder and/or condition associated with alpha-synuclein; and/or
c. no significant change of levels of alpha-synuclein in the later sample compared with one or more earlier samples are indicative of lack of progression of a disease, disorder and/or condition associated with alpha-synuclein; or
a method for selecting a therapy for treatment of a disease, disorder and/or condition associated with alpha-synuclein comprising contacting samples from a subject, taken before and after treatment with the therapy, with an alpha-synuclein biparatopic antibody or functional fragment thereof, or a mixture comprising at least two monospecific antibodies or functional fragments thereof, according to claim 65, wherein;
a. lower levels of alpha-synuclein in the sample taken after treatment compared with the sample taken before treatment are indicative of successful treatment of a disease, disorder and/or condition associated with alpha-synuclein and thus the therapy is selected for treatment;
b. no significant change of levels of alpha-synuclein in the sample taken after treatment compared with the sample taken before treatment are indicative of successful treatment of a disease, disorder and/or condition associated with alpha-synuclein and thus the therapy is selected for treatment;
c. a decline in the rate of increase of levels of alpha-synuclein between samples taken during treatment compared with samples taken before treatment are indicative of successful treatment of a disease, disorder and/or condition associated with alpha-synuclein and thus the therapy is selected for treatment;
d. higher levels of alpha-synuclein in the sample taken after treatment compared with the sample taken before treatment are indicative of unsuccessful treatment of a disease, disorder and/or condition associated with alpha-synuclein and thus the therapy is not selected for treatment; or
e. no decline in the rate of increase of levels of alpha-synuclein between samples taken during treatment compared with samples taken before treatment are indicative of unsuccessful treatment of a disease, disorder and/or condition associated with alpha-synuclein and thus the therapy is not selected for treatment; or
a method for assessing a candidate therapy for a disease, disorder and/or condition associated with alpha-synuclein, the method comprising, following treatment of one or more subjects, contacting samples from the one or more treated subjects with an alpha-synuclein biparatopic antibody or functional fragment thereof, or a mixture comprising at least two monospecific antibodies or functional fragments thereof, according to claim 65, wherein lower levels of alpha-synuclein in the samples compared with levels in corresponding samples from subjects not treated with the therapy are indicative of successful treatment of a disease, disorder and/or condition associated with alpha-synuclein;
optionally wherein the method is performed at multiple time points in matched samples between the treatment and placebo groups in order to monitor the effectiveness of the candidate therapy over a defined time period;
optionally wherein the method comprises contacting samples, from the one or more treated subjects and the subjects not treated with the therapy, with an alpha-synuclein biparatopic antibody or functional fragment thereof, or a mixture comprising at least two monospecific antibodies or functional fragments thereof, according to claim 65 prior to treatment, with the therapy or placebo respectively, to determine base levels of alpha-synuclein, optionally wherein the sample or samples is selected from saliva, urine, nasal secretion, blood, brain and/or CSF, brain and/or interstitial fluid (ISF).

95. The method of claim 94, wherein the disease, disorder and/or condition associated with alpha-synuclein is:
a synucleinopathy; or
is selected from the group consisting of Parkinson's disease (sporadic, familial with alpha-synuclein mutations, familial with mutations other than alpha-synuclein, pure autonomic failure and Lewy body dysphagia), Lewy Body dementia (LBD; including dementia with Lewy bodies (DLB) (“pure” Lewy body dementia), Parkinson's disease dementia (PDD)), or Diffuse Lewy Body Disease, sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.


SEQUENCE LISTING (863 sequences)

SEQ ID NO: 1 - protein, 140 aa, Unknown
  FL-alpha-synuclein from an organism of the kingdom Mammalia
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 5 aa, Unknown
  Binding Epitope from an organism of the kingdom Mammalia
GVLYV

SEQ ID NO: 3 - protein, 7 aa, Unknown
  Binding Epitope from an organism of the kingdom Mammalia
GVATVAE

SEQ ID NO: 4 - protein, 10 aa, Unknown
  Binding Epitope from an organism of the kingdom Mammalia
NVGGAVVTGV

SEQ ID NO: 5 - protein, 17 aa, Unknown
  Binding Epitope from an organism of the kingdom Mammalia
NVGGAVVTGVTAVAQKT

SEQ ID NO: 6 - skipped

SEQ ID NO: 7 - protein, 8 aa, Unknown
  Binding Epitope from an organism of the kingdom Mammalia
AYEMPSEE

SEQ ID NO: 8 - protein, 8 aa, Unknown
  Binding Epitope from an organism of the kingdom Mammalia
PSEEGYQD

SEQ ID NO: 9 - protein, 10 aa, Unknown
  Binding Epitope from an organism of the kingdom Mammalia
EGYQDYEPEA

SEQ ID NO: 10 - protein, 121 aa, Artificial Sequence
  ACI-7067-1101C8-Ab2 1101C8F7 VH
EVQLVESGGGLVQPKGSLKLSCAASGFSFNIYAMNWVRQAPGKGLEWVARIRSKSNNYATYYADSVKDRFTISRADSESMLYLQMNNLKTEDTAMYYCVRVGLRFYAMDYWGQGTSVTVSS

SEQ ID NO: 11 - protein, 5 aa, Artificial Sequence
  ACI-7067-1101C8-Ab2 1101C8F7 VH CDR1
IYAMN

SEQ ID NO: 12 - protein, 19 aa, Artificial Sequence
  ACI-7067-1101C8-Ab2 1101C8F7 VH CDR2
RIRSKSNNYATYYADSVKD

SEQ ID NO: 13 - protein, 10 aa, Artificial Sequence
  ACI-7067-1101C8-Ab2 1101C8F7 VH CDR3
VGLRFYAMDY

SEQ ID NO: 14 - protein, 112 aa, Artificial Sequence
  ACI-7067-1101C8-Ab2 1101C8F7 VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSQGPLTFGAGTKLELK

SEQ ID NO: 15 - protein, 16 aa, Artificial Sequence
  ACI-7067-1101C8-Ab2 1101C8F7 VL CDR1
RSSQSIVHSNGNTYLE

SEQ ID NO: 16 - protein, 7 aa, Artificial Sequence
  ACI-7067-1101C8-Ab2 1101C8F7 VL CDR2
KVSNRFS

SEQ ID NO: 17 - protein, 9 aa, Artificial Sequence
  ACI-7067-1101C8-Ab2 1101C8F7 VL CDR3
FQGSQGPLT

SEQ ID NO: 18 - DNA, 363 bp, Artificial Sequence
  ACI-7067-1101C8-Ab2 1101C8F7 VH
gaggtgcagcttgttgagtctggtggaggattggtgcagcctaaagggtcattgaaactctcatgtgcagcctctggattcagcttcaatatctacgccatgaactgggtccgccaggctccaggaaagggtttggaatgggttgctcgcataagaagtaaaagtaataattatgcaacatattatgccgattcagtgaaagacagattcaccatctccagagctgattcagaaagcatgctctatctgcaaatgaacaacttgaaaactgaggacacagccatgtattactgtgtaagggtgggcctacggttctatgctatggactactggggtcaaggcacctcagtcaccgtctcctca

SEQ ID NO: 19 - DNA, 336 bp, Artificial Sequence
  ACI-7067-1101C8-Ab2 1101C8F7 VL
gatgttttgatgacccaaactccactctccctgcctgtcagtcttggagatcaagcctccatctcttgcagatctagtcagagcattgtacatagtaatggaaacacctatttagaatggtacttgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacaaggtccgctcacgttcggtgctgggaccaagctggagctgaaa

SEQ ID NO: 20 - protein, 114 aa, Artificial Sequence
  ACI-7067-1102G3-Ab1 1102G3F2 VH
EVKLEESGGGLVQPGGSMKLSCAASGFTFSDAWMNWVRQSPEKGLEWVAEIRNKAHNHATYYAESVKGRFTISGDDSKSSVYLQMNNLRAEDTGIYYCTIYSYWGQGTLVTVSA

SEQ ID NO: 21 - protein, 4 aa, Artificial Sequence
  ACI-7067-1102G3-Ab1 1102G3F2 VH CDR1
DAWM

SEQ ID NO: 22 - protein, 19 aa, Artificial Sequence
  ACI-7067-1102G3-Ab1 1102G3F2 VH CDR2
EIRNKAHNHATYYAESVKG

SEQ ID NO: 23 - skipped

SEQ ID NO: 24 - protein, 107 aa, Artificial Sequence
  ACI-7067-1102G3-Ab1 1102G3F2 VL
SIVMTQTPKFLLVSAGDRVTITCKASQSVTKDVAWYQQKPGQSPKLLIYSTSNRYSGVPDRFTGSGYGTDFTFTINTVQTEDLAVYFCQQDYRIPYTFGGGTKLEIK

SEQ ID NO: 25 - protein, 11 aa, Artificial Sequence
  ACI-7067-1102G3-Ab1 1102G3F2 VL CDR1
KASQSVTKDVA

SEQ ID NO: 26 - protein, 7 aa, Artificial Sequence
  ACI-7067-1102G3-Ab1 1102G3F2 VL CDR2
STSNRYS

SEQ ID NO: 27 - protein, 9 aa, Artificial Sequence
  ACI-7067-1102G3-Ab1 1102G3F2 VL CDR3
QQDYRIPYT

SEQ ID NO: 28 - DNA, 342 bp, Artificial Sequence
  ACI-7067-1102G3-Ab1 1102G3F2 VH
gaagtgaagcttgaggagtctggaggaggcttggtgcaacctggaggatccatgaaactctcttgtgctgcctctggattcacttttagtgacgcctggatgaactgggtccgccagtctccagagaaggggcttgagtgggttgctgaaattagaaacaaagctcataatcatgcaacatactatgctgagtctgtgaaagggaggttcaccatctcaggagatgattccaaaagtagtgtctacctgcaaatgaacaacttaagagctgaagacactggcatttattactgtaccatttactcttattggggccaagggactctggtcactgtctctgca

SEQ ID NO: 29 - DNA, 321 bp, Artificial Sequence
  ACI-7067-1102G3-Ab1 1102G3F2 VL
agtattgtgatgacccagactcccaaattcctgcttgtatcagcaggagacagggttaccataacctgcaaggccagtcagagtgtgactaaagatgtagcttggtaccaacagaagccagggcagtctcctaaactgctgatatactctacatccaatcgctacagtggagtccctgatcgcttcactggcagtggatatgggacggatttcactttcaccatcaatactgtgcagactgaagacctggcagtttatttctgtcagcaggattacaggattccgtacacgttcggaggggggaccaagctggaaataaaa

SEQ ID NO: 30 - protein, 120 aa, Artificial Sequence
  ACI-7067-1106A8-Ab2 1106A8H3 VH
EVQLVESGGGLVQPKGSLKLSCAASGFTFNTYAMHWVRQAPGKGLEWVARIRSKGSNYATNYADSVKDRFTISRDDSQSMLYLQMNNLKTEDTAMYYCVRGHGSSYFSYWGQGTLVTVSA

SEQ ID NO: 31 - protein, 5 aa, Artificial Sequence
  ACI-7067-1106A8-Ab2 1106A8H3 VH CDR1
TYAMH

SEQ ID NO: 32 - protein, 19 aa, Artificial Sequence
  ACI-7067-1106A8-Ab2 1106A8H3 VH CDR2
RIRSKGSNYATNYADSVKD

SEQ ID NO: 33 - protein, 9 aa, Artificial Sequence
  ACI-7067-1106A8-Ab2 1106A8H3 VH CDR3
GHGSSYFSY

SEQ ID NO: 34 - protein, 106 aa, Artificial Sequence
  ACI-7067-1106A8-Ab2 1106A8H3 VL
QIVLTQSPAIMSASPGEKVTMTCSASSSVSYMHWYQQKSGTSPKRWIYDTSNLASGVPARFSGSGSGTSYSLTISSMEAEDAATYYCQQWNSHPPTFGAGTKLELK

SEQ ID NO: 35 - protein, 8 aa, Artificial Sequence
  ACI-7067-1106A8-Ab2 1106A8H3 VL CDR1
SASSSVSY

SEQ ID NO: 36 - protein, 7 aa, Artificial Sequence
  ACI-7067-1106A8-Ab2 1106A8H3 VL CDR2
DTSNLAS

SEQ ID NO: 37 - protein, 9 aa, Artificial Sequence
  ACI-7067-1106A8-Ab2 1106A8H3 VL CDR3
QQWNSHPPT

SEQ ID NO: 38 - DNA, 360 bp, Artificial Sequence
  ACI-7067-1106A8-Ab2 1106A8H3 VH
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaggtagtaattatgcaacaaattatgccgattcagtgaaagacagattcaccatctccagagatgattcgcaaagcatgctctatctgcaaatgaacaacctgaaaactgaggacacagccatgtattactgtgtgagaggacacggtagtagctacttttcttactggggccaagggactctggtcactgtctctgca

SEQ ID NO: 39 - DNA, 318 bp, Artificial Sequence
  ACI-7067-1106A8-Ab2 1106A8H3 VL
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaatctggcttctggagtccctgctcgcttcagtggcagtgggtctgggacctcttactctctcacaatcagcagcatggaggctgaagatgctgccacttattactgccagcagtggaatagtcacccacccacgttcggtgctgggaccaagctggaactgaaa

SEQ ID NO: 40 - protein, 113 aa, Artificial Sequence
  ACI-7067-1107G5-Ab2 1107G5B6 VH
QVQLQQPGTELVKPGASVKLSCKASGYTFTKYWMHWVKQRPGQGLEWIGNINPNNGDTNYNEKFKSKATLTVDKSSSTAYMQLSSLTSEDSAVYYCAIAMDYWGQGTSVTVSS

SEQ ID NO: 41 - protein, 5 aa, Artificial Sequence
  ACI-7067-1107G5-Ab2 1107G5B6 VH CDR1
KYWMH

SEQ ID NO: 42 - protein, 17 aa, Artificial Sequence
  ACI-7067-1107G5-Ab2 1107G5B6 VH CDR2
NINPNNGDTNYNEKFKS

SEQ ID NO: 43 - protein, 4 aa, Artificial Sequence
  ACI-7067-1107G5-Ab2 1107G5B6 VH CDR3
AMDY

SEQ ID NO: 44 - protein, 107 aa, Artificial Sequence
  ACI-7067-1107G5-Ab2 1107G5B6 VL
DIQMTQSPSSLSAFLGERVSLTCRASQDIGNNLNWFQQEPDGTIKRLIYATSSLDSGVPKRFSGSRSGSEYSLTISSLESEDFVDYYCLQFGSSPLTFGAGTKLELK

SEQ ID NO: 45 - protein, 11 aa, Artificial Sequence
  ACI-7067-1107G5-Ab2 1107G5B6 VL CDR1
RASQDIGNNLN

SEQ ID NO: 46 - protein, 7 aa, Artificial Sequence
  ACI-7067-1107G5-Ab2 1107G5B6 VL CDR2
ATSSLDS

SEQ ID NO: 47 - protein, 9 aa, Artificial Sequence
  ACI-7067-1107G5-Ab2 1107G5B6 VL CDR3
LQFGSSPLT

SEQ ID NO: 48 - DNA, 339 bp, Artificial Sequence
  ACI-7067-1107G5-Ab2 1107G5B6 VH
caggtccaactgcagcagcctgggactgaactggtgaagcctggggcttcagtgaagctgtcctgcaaggcttctggctacaccttcaccaaatactggatgcactgggtgaagcagaggcctggacaaggccttgagtggattggaaatattaatcctaacaatggtgatactaactacaatgagaagttcaagagcaaggccacactgactgtagacaaatcctccagcacagcctacatgcagctcagcagtctgacatctgaggactctgcggtctattattgtgcaattgctatggactactggggtcaaggaacctcagtcaccgtctcctca

SEQ ID NO: 49 - DNA, 321 bp, Artificial Sequence
  ACI-7067-1107G5-Ab2 1107G5B6 VL
gacatccagatgacccagtctccatcctccttatctgcctttctgggagaaagagtcagtctcacttgtcgggcaagtcaggacattggtaataacttaaactggtttcagcaggaaccagatggaactattaaacgtctgatctacgccacatccagtttagattctggtgtccccaaaaggttcagtggcagtaggtctgggtcagaatattctctcaccatcagcagccttgagtctgaagattttgtagactattactgtctacaatttggtagttctccgctcacgttcggtgctgggaccaagctggagctgaaa

SEQ ID NO: 50 - protein, 114 aa, Artificial Sequence
  ACI-7067-1108H1-Ab1 1108H1E1 VH
EVKLVESGGGLVQPGGSMKLSCTASGFTFSDAWMNWVRQSPEKGLEWVAEIRNKAHNHATNYAESVKGRFTISGDDSKSSVYLQMNNLRAEDTGIYYCTIYSFWGQGTLVTVSA

SEQ ID NO: 51 - protein, 4 aa, Artificial Sequence
  ACI-7067-1108H1-Ab1 1108H1E1 VH CDR1
DAWM

SEQ ID NO: 52 - protein, 19 aa, Artificial Sequence
  ACI-7067-1108H1-Ab1 1108H1E1 VH CDR2
EIRNKAHNHATNYAESVKG

SEQ ID NO: 53 - skipped

SEQ ID NO: 54 - protein, 107 aa, Artificial Sequence
  ACI-7067-1108H1-Ab1 1108H1E1 VL
SIVMTQTPKFLLVSAGDRVTITCKASQSVTNYVAWYHQKPGQSPKLLIYSASNRYSGVPDRFTGSGYGTDFTFTINTVQTEDLAVYFCQQDYRIPYTFGGGTKLEIK

SEQ ID NO: 55 - protein, 11 aa, Artificial Sequence
  ACI-7067-1108H1-Ab1 1108H1E1 VL CDR1
KASQSVTNYVA

SEQ ID NO: 56 - protein, 7 aa, Artificial Sequence
  ACI-7067-1108H1-Ab1 1108H1E1 VL CDR2
SASNRYS

SEQ ID NO: 57 - protein, 9 aa, Artificial Sequence
  ACI-7067-1108H1-Ab1 1108H1E1 VL CDR3
QQDYRIPYT

SEQ ID NO: 58 - DNA, 342 bp, Artificial Sequence
  ACI-7067-1108H1-Ab1 1108H1E1 VH
gaggtgaagctggtggagtctggaggaggcttggtgcaacctggaggatccatgaaactctcttgtactgcctctggattcacttttagtgacgcctggatgaactgggtccgccagtctccagagaaggggcttgagtgggttgctgaaattagaaacaaagctcataatcatgcaacaaactatgctgagtctgtgaaggggaggttcaccatctcaggagatgattccaaaagtagtgtctacctgcaaatgaacaacttaagagctgaagacactggcatttattactgtaccatttactctttttggggccaagggactctggtcactgtctctgca

SEQ ID NO: 59 - DNA, 321 bp, Artificial Sequence
  ACI-7067-1108H1-Ab1 1108H1E1 VL
agtattgtgatgacccagactcccaaattcctgcttgtatcagcaggagacagggttaccataacctgcaaggccagtcagagtgtgactaattatgtagcttggtaccatcagaagccagggcagtctcctaaactgctgatatactctgcatccaatcgctacagtggagtccctgatcgcttcactggcagtggatatgggacggatttcactttcaccatcaatactgtgcagactgaagacctggcagtttatttctgtcagcaggattacaggattccgtacacgttcggaggggggactaagctggaaataaaa

SEQ ID NO: 60 - protein, 113 aa, Artificial Sequence
  ACI-7067-1111B12-Ab2 1111B12H10 VH
QVQLLQPGTALVMPGASVKLSCKASGYTFTTYWMHWVKQRPGQGLEWIGNINPINGGSNYNEKFKSKASLTVDKSSSTAYMQLSSLTSEDSAVYYCVIAMDYWGQGTSVTVSS

SEQ ID NO: 61 - protein, 5 aa, Artificial Sequence
  ACI-7067-1111B12-Ab2 1111B12H10 VH CDR1
TYWMH

SEQ ID NO: 62 - protein, 17 aa, Artificial Sequence
  ACI-7067-1111B12-Ab2 1111B12H10 VH CDR2
NINPINGGSNYNEKFKS

SEQ ID NO: 63 - protein, 4 aa, Artificial Sequence
  ACI-7067-1111B12-Ab2 1111B12H10 VH CDR3
AMDY

SEQ ID NO: 64 - protein, 107 aa, Artificial Sequence
  ACI-7067-1111B12-Ab2 1111B12H10 VL
DIQMTQSPSSLSASLGERVSLTCRASQDIGISLNWFQQEPDGTIKRLIYATSSLDSGVPKRFSGNRSGSDYSLTISSLESEDFADYYCLQFASSPLTFGAGTKLELK

SEQ ID NO: 65 - protein, 11 aa, Artificial Sequence
  ACI-7067-1111B12-Ab2 1111B12H10 VL CDR1
RASQDIGISLN

SEQ ID NO: 66 - protein, 7 aa, Artificial Sequence
  ACI-7067-1111B12-Ab2 1111B12H10 VL CDR2
ATSSLDS

SEQ ID NO: 67 - protein, 9 aa, Artificial Sequence
  ACI-7067-1111B12-Ab2 1111B12H10 VL CDR3
LQFASSPLT

SEQ ID NO: 68 - DNA, 339 bp, Artificial Sequence
  ACI-7067-1111B12-Ab2 1111B12H10 VH
caggtccaactgctgcagcctgggactgcactggtgatgcctggggcttcagtgaagctgtcctgcaaggcttctggctacaccttcaccacctactggatgcactgggtgaagcagaggcctggacaaggccttgagtggattggaaatattaatcctatcaatggtggtagtaactacaatgagaagttcaagagcaaggcctcactgactgtagacaagtcctccagcacagcctacatgcagctcagcagcctgacatctgaggactctgcggtctattattgtgtcattgctatggactactggggtcaaggaacctcagtcaccgtctcctca

SEQ ID NO: 69 - DNA, 321 bp, Artificial Sequence
  ACI-7067-1111B12-Ab2 1111B12H10 VL
gacatccagatgacccagtctccatcctccttatctgcctctctgggagaaagagtcagtctcacatgtcgggcaagtcaggacattggtattagcttaaactggtttcagcaggaaccagatggaactattaaacgcctgatctacgccacatccagtttagattctggtgtccccaaaaggttcagtggcaataggtctgggtcagattattctctcaccatcagtagccttgagtctgaagattttgcagactattactgtctacaatttgctagttctccgctcacgttcggtgctgggaccaagctggagctgaaa

SEQ ID NO: 70 - protein, 114 aa, Artificial Sequence
  ACI-7067-1112H8-Ab2 1112H8C12 VH
EVKLEESGGGLVQPGGSMKLSCAASGFTFTDAWMNWVRQSPEKGLEWIAEIRNKAHNYATYYAESVKGRFDISGDDSKSSVYLQMNNLRVEDTGIYYCTIYSYWGPGTLVTVSA

SEQ ID NO: 71 - protein, 4 aa, Artificial Sequence
  ACI-7067-1112H8-Ab2 1112H8C12 VH CDR1
DAWM

SEQ ID NO: 72 - protein, 19 aa, Artificial Sequence
  ACI-7067-1112H8-Ab2 1112H8C12 VH CDR2
EIRNKAHNYATYYAESVKG

SEQ ID NO: 73 - skipped

SEQ ID NO: 74 - protein, 107 aa, Artificial Sequence
  ACI-7067-1112H8-Ab2 1112H8C12 VL
SIVMTQTPKFLLMSPGDRVTMTCTASQSVSNYVAWYQQKPGQSPKLLIYSASNRFTGVPDRFTGSGYGTDFTFTINTVQTEDMAVYFCQQDYTSPYTFGGGTKLEIK

SEQ ID NO: 75 - protein, 11 aa, Artificial Sequence
  ACI-7067-1112H8-Ab2 1112H8C12 VL CDR1
TASQSVSNYVA

SEQ ID NO: 76 - protein, 7 aa, Artificial Sequence
  ACI-7067-1112H8-Ab2 1112H8C12 VL CDR2
SASNRFT

SEQ ID NO: 77 - protein, 9 aa, Artificial Sequence
  ACI-7067-1112H8-Ab2 1112H8C12 VL CDR3
QQDYTSPYT

SEQ ID NO: 78 - DNA, 342 bp, Artificial Sequence
  ACI-7067-1112H8-Ab2 1112H8C12 VH
gaagtgaagcttgaggagtctggaggaggcttggtgcaacctggaggatccatgaaactctcttgtgctgcctctggattcacttttactgacgcctggatgaactgggtccgccagtctccagaaaaggggcttgagtggattgctgaaattagaaacaaagctcataattatgcaacatactatgctgagtctgtgaaagggaggttcgacatctcaggagatgattccaaaagtagtgtctacctgcaaatgaacaacttgagagttgaagacactggcatttattactgtaccatttactcttactggggcccagggactctggtcactgtctctgca

SEQ ID NO: 79 - DNA, 321 bp, Artificial Sequence
  ACI-7067-1112H8-Ab2 1112H8C12 VL
agtattgtgatgacccagactcccaaattcctgcttatgtcaccaggagacagggttaccatgacctgcacggccagtcagagtgtgagtaattatgtggcttggtaccaacagaagccagggcagtctcctaaactgctgatatactctgcatccaatcgcttcactggagtccctgatcgcttcactggcagtggatatgggacggatttcactttcaccatcaacactgtgcagactgaagacatggcagtttatttctgtcagcaggattacacctctccgtacacgttcgggggggggaccaagctggaaataaaa

SEQ ID NO: 80 - protein, 120 aa, Artificial Sequence; identical to SEQ ID NO: 30
  ACI-7067-1108B11-Ab2 1108B11D3 VH

SEQ ID NO: 81 - protein, 5 aa, Artificial Sequence
  ACI-7067-1108B11-Ab2 1108B11D3 VH CDR1
TYAMH

SEQ ID NO: 82 - protein, 19 aa, Artificial Sequence
  ACI-7067-1108B11-Ab2 1108B11D3 VH CDR2
RIRSKGSNYATNYADSVKD

SEQ ID NO: 83 - protein, 9 aa, Artificial Sequence
  ACI-7067-1108B11-Ab2 1108B11D3 VH CDR3
GHGSSYFSY

SEQ ID NO: 84 - protein, 106 aa, Artificial Sequence
  ACI-7067-1108B11-Ab2 1108B11D3 VL
QIVLTQSPAIMSASPGERITMTCSANSSVTYMHWYQQKSGTSPKRWIYDTSNLASGVPARFSGSGSGTSYSLTISSMEAEDAATYYCQQWKSHPPTFGAGTKLELK

SEQ ID NO: 85 - protein, 10 aa, Artificial Sequence
  ACI-7067-1108B11-Ab2 1108B11D3 VL CDR1
SANSSVTYMH

SEQ ID NO: 86 - protein, 7 aa, Artificial Sequence
  ACI-7067-1108B11-Ab2 1108B11D3 VL CDR2
DTSNLAS

SEQ ID NO: 87 - protein, 9 aa, Artificial Sequence
  ACI-7067-1108B11-Ab2 1108B11D3 VL CDR3
QQWKSHPPT

SEQ ID NO: 88 - DNA, 360 bp, Artificial Sequence; identical to SEQ ID NO: 38
  ACI-7067-1108B11-Ab2 1108B11D3 VH

SEQ ID NO: 89 - DNA, 318 bp, Artificial Sequence
  ACI-7067-1108B11-Ab2 1108B11D3 VL
caaattgttctcacccagtctccagcaatcatgtctgcatctccaggggagaggatcaccatgacctgcagtgccaactcaagtgttacttacatgcactggtaccagcagaagtcaggcacctcccccaaaagatggatttatgacacatccaatctggcttctggagtccctgctcgcttcagtggcagtgggtctgggacctcttactctctcacaatcagcagcatggaggctgaagatgctgccacttattactgccagcagtggaaaagtcacccacccacgttcggtgctgggaccaagctggaactgaaa

SEQ ID NO: 90 - protein, 119 aa, Artificial Sequence
  ACI-7067-1113D10-Ab1 1113D10E3D5 VH
EVQLVESGGGLVQPKGSLKLSCAASGFTFNTYALHWVRQAPGKGLEWVARIRSKSSNYATYYADSVKDRFTISRDDSQSMLYLQMNNLKTEDTGMYYCVRGGVSPFDYWGQGTTLTVSS

SEQ ID NO: 91 - protein, 5 aa, Artificial Sequence
  ACI-7067-1113D10-Ab1 1113D10E3D5 VH CDR1
TYALH

SEQ ID NO: 92 - protein, 19 aa, Artificial Sequence
  ACI-7067-1113D10-Ab1 1113D10E3D5 VH CDR2
RIRSKSSNYATYYADSVKD

SEQ ID NO: 93 - protein, 8 aa, Artificial Sequence
  ACI-7067-1113D10-Ab1 1113D10E3D5 VH CDR3
GGVSPFDY

SEQ ID NO: 94 - protein, 106 aa, Artificial Sequence
  ACI-7067-1113D10-Ab1 1113D10E3D5 VL
QIVLTQSPAIMSASPGEKVTMTCSASSSVSYMHWYQQKSGTSPKRWIYDTSKLASGVPARFSGSGSGTSYSLTISSMEAEDSATYYCQQWSNNPPTFGGGTKLEIK

SEQ ID NO: 95 - protein, 10 aa, Artificial Sequence
  ACI-7067-1113D10-Ab1 1113D10E3D5 VL CDR1
SASSSVSYMH

SEQ ID NO: 96 - protein, 7 aa, Artificial Sequence
  ACI-7067-1113D10-Ab1 1113D10E3D5 VL CDR2
DTSKLAS

SEQ ID NO: 97 - protein, 9 aa, Artificial Sequence
  ACI-7067-1113D10-Ab1 1113D10E3D5 VL CDR3
QQWSNNPPT

SEQ ID NO: 98 - DNA, 357 bp, Artificial Sequence
  ACI-7067-1113D10-Ab1 1113D10E3D5 VH
gaggtgcagcttgttgagtctggtggaggattggtgcagcctaaaggatcattgaaactctcatgtgccgcctctggtttcaccttcaatacctatgccctgcactgggtccgccaggctccaggaaagggtttggaatgggttgctcgcataagaagtaaaagtagtaattatgcaacatattatgccgattcagtgaaagacagattcaccatctccagagatgattcacaaagcatgctctatctgcaaatgaacaacctgaaaactgaggacacaggcatgtattactgtgtaagagggggtgtttctccctttgactactggggccaaggcaccactctcacagtctcctca

SEQ ID NO: 99 - DNA, 318 bp, Artificial Sequence
  ACI-7067-1113D10-Ab1 1113D10E3D5 VL
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagattctgccacttattactgccagcagtggagtaataacccaccgacgttcggtggaggcaccaagctggaaatcaaa

SEQ ID NO: 100 - protein, 113 aa, Artificial Sequence
  ACI-7067-1116F2-Ab1 1116F2A2 VH
DVQLQESGPGFVKPSQSLSLTCSVTGYSITRGFYWNWIRQFPGNKLEWMGYISDDGNSNYNPSLKNRISITRDTFKNQVFLRLNSVTTEDTATYYCTRGDLLWGQGTTLTVSS

SEQ ID NO: 101 - protein, 6 aa, Artificial Sequence
  ACI-7067-1116F2-Ab1 1116F2A2 VH CDR1
RGFYWN

SEQ ID NO: 102 - protein, 16 aa, Artificial Sequence
  ACI-7067-1116F2-Ab1 1116F2A2 VH CDR2
YISDDGNSNYNPSLKN

SEQ ID NO: 103 - protein, 4 aa, Artificial Sequence
  ACI-7067-1116F2-Ab1 1116F2A2 VH CDR3
GDLL

SEQ ID NO: 104 - protein, 112 aa, Artificial Sequence
  ACI-7067-1116F2-Ab1 1116F2A2 VL
DVVMTQTALTLSVTIGQPASISCKSSQSLLDSDGETYLNWLLQRPGQSPKRLIYLVSKLDSGVPDRFTGSGSGTDFALKISRVEAEDLGIYYCWQGTHFPQTFGGGTKLEIK

SEQ ID NO: 105 - protein, 16 aa, Artificial Sequence
  ACI-7067-1116F2-Ab1 1116F2A2 VL CDR1
KSSQSLLDSDGETYLN

SEQ ID NO: 106 - protein, 7 aa, Artificial Sequence
  ACI-7067-1116F2-Ab1 1116F2A2 VL CDR2
LVSKLDS

SEQ ID NO: 107 - protein, 9 aa, Artificial Sequence
  ACI-7067-1116F2-Ab1 1116F2A2 VL CDR3
WQGTHFPQT

SEQ ID NO: 108 - DNA, 339 bp, Artificial Sequence
  ACI-7067-1116F2-Ab1 1116F2A2 VH
gatgtacaacttcaggagtcaggacctggcttcgtgaaaccttctcagtctctgtctctcacctgctctgtcactggctactcaataaccagaggtttttactggaactggatccgacagtttccaggaaacaaactggaatggatgggctacataagtgacgatggtaatagtaactacaatccctctctcaaaaatcgaatctccatcactcgtgacacatttaagaatcaggttttcctgaggttgaactctgtgactactgaggacactgccacatactattgtacaagaggagatctactttggggccaaggcaccactctcacagtctcctca

SEQ ID NO: 109 - DNA, 336 bp, Artificial Sequence
  ACI-7067-1116F2-Ab1 1116F2A2 VL
gatgttgtgatgacccagactgcactcactttgtcggttaccattggacaaccagcctccatctcttgcaagtcaagtcaaagcctcttagatagtgatggagagacatatttgaattggttgttacagaggccaggccagtctccaaagcgcctaatctatctggtgtctaaactggactctggagtccctgacaggttcactggtagtggatcagggacagatttcgcactgaaaatcagcagagtggaggctgaggacttgggaatttattattgctggcaaggtacacattttcctcagacgttcggtggaggcaccaagctggaaatcaaa

SEQ ID NO: 110 - protein, 123 aa, Artificial Sequence
  ACI-7067-1206E5-Ab1 1206E5D2 VH
QVQLQQSGPELVKPGASVKMSCKASGYTFTDYVISWVKQGTGQGLEWIGEIYPGNDSTYYNEKFKGKATLTADKSSNTAYMQLSSLTSEDSAVYFCAREGVSNGYLYLSMDYWGQGTSVTVSS

SEQ ID NO: 111 - protein, 5 aa, Artificial Sequence
  ACI-7067-1206E5-Ab1 1206E5D2 VH CDR1
DYVIS

SEQ ID NO: 112 - protein, 17 aa, Artificial Sequence
  ACI-7067-1206E5-Ab1 1206E5D2 VH CDR2
EIYPGNDSTYYNEKFKG

SEQ ID NO: 113 - protein, 14 aa, Artificial Sequence
  ACI-7067-1206E5-Ab1 1206E5D2 VH CDR3
EGVSNGYLYLSMDY

SEQ ID NO: 114 - protein, 112 aa, Artificial Sequence
  ACI-7067-1206E5-Ab1 1206E5D2 VL
DVLMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPWTFGGGTKLEIK

SEQ ID NO: 115 - protein, 16 aa, Artificial Sequence
  ACI-7067-1206E5-Ab1 1206E5D2 VL CDR1
KSSQSLLYSNGKTYLN

SEQ ID NO: 116 - protein, 7 aa, Artificial Sequence
  ACI-7067-1206E5-Ab1 1206E5D2 VL CDR2
LVSKLDS

SEQ ID NO: 117 - protein, 9 aa, Artificial Sequence
  ACI-7067-1206E5-Ab1 1206E5D2 VL CDR3
VQGTHFPWT

SEQ ID NO: 118 - DNA, 369 bp, Artificial Sequence
  ACI-7067-1206E5-Ab1 1206E5D2 VH
caggttcagctgcagcagtctggacctgagctggtgaagcctggggcttcagtgaagatgtcctgcaaggcttctggatacacattcactgactatgttataagctgggtgaagcagggaactggacagggccttgagtggattggagagatttatcctggaaatgatagtacttactacaatgagaagttcaagggcaaggccacactgactgcagacaaatcctccaacacagcctacatgcagctcagcagcctgacatctgaggactctgcggtctatttctgtgcaagagagggggtctctaatggttacctatatttgtctatggactactggggtcaaggaacctcagtcaccgtctcctca

SEQ ID NO: 119 - DNA, 336 bp, Artificial Sequence
  ACI-7067-1206E5-Ab1 1206E5D2 VL
gatgttttgatgacccaaactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacattttccgtggacgttcggtggaggcaccaagctggaaatcaaa

SEQ ID NO: 120 - protein, 14 aa, Artificial sequence
  Artificial Peptide
MDVFMKGLSKAKEG

SEQ ID NO: 121 - protein, 15 aa, Unknown
  Binding Epitope from an organism of the kingdom Mammalia
MDVFMKGLSKAKEGV

SEQ ID NO: 122 - protein, 15 aa, Artificial sequence
  Artificial Peptide
KAKEGVVAAAEKTKQ

SEQ ID NO: 123 - protein, 15 aa, Artificial sequence
  Artificial peptide
AEKTKQGVAEAAGKT

SEQ ID NO: 124 - protein, 15 aa, Artificial sequence
  Artificial Peptide
EAAGKTKEGVLYVGS

SEQ ID NO: 125 - protein, 15 aa, Artificial sequence
  Artificial Peptide
VLYVGSKTKEGVVHG

SEQ ID NO: 126 - protein, 15 aa, Artificial sequence
  Artificial Peptide
EGVVHGVATVAEKTK

SEQ ID NO: 127 - protein, 15 aa, Artificial sequence
  Artificial Peptide
VAEKTKEQVTNVGGA

SEQ ID NO: 128 - protein, 15 aa, Unknown
  Binding epitope from an organism of the kingdom Mammalia
TNVGGAVVTGVTAVA

SEQ ID NO: 129 - protein, 15 aa, Artificial sequence
  Artificial Peptide
GVTAVAQKTVEGAGS

SEQ ID NO: 130 - protein, 15 aa, Unknown
  Binding epitope from an orgnism of the kingdom Mammalia
VEGAGSIAAATGFVK

SEQ ID NO: 131 - protein, 15 aa, Unknown
  Binding epitope from an organism of the kingdom Mammalia
ATGFVKKDQLGKNEE

SEQ ID NO: 132 - protein, 15 aa, Artificial sequence
  Artificial Peptide
LGKNEEGAPQEGILE

SEQ ID NO: 133 - protein, 15 aa, Artificial sequence
  Artificial Peptide
QEGILEDMPVDPDNE

SEQ ID NO: 134 - protein, 15 aa, Unknown
  Binding epitope from an organism of the kingdom Mammalia
VDPDNEAYEMPSEEG

SEQ ID NO: 135 - protein, 14 aa, Unknown
  Binding Epitope from an organism of the kingdom Mammalia
MPSEEGYQDYEPEA

SEQ ID NO: 136 - protein, 8 aa, Unknown
  Binding Epitope from an organism of the kingdom Mammalia
GVATVAEK

SEQ ID NO: 137 - protein, 40 aa, Unknown
  Binding Epitope from an organism of the kingdom Mammalia
TVEGAGSIAAATGFVKKDQLGKNEEGAPQEGILEDMPVDP

SEQ ID NO: 138 - protein, 31 aa, Unknown
  Binding Epitope from an organism of the kingdom Mammalia
VVAAAEKTKQGVAEAAGKTKEGVLYVGSKTK

SEQ ID NO: 139 - protein, 23 aa, Unknown
  Binding Epitope from an organism of the kingdom Mammalia
EAAGKTKEGVLYVGSKTKEGVVH

SEQ ID NO: 140 - protein, 121 aa, Artificial Sequence
  ACI-7079-2501G2-Ab2 2501G2E5 VH
EVQLVESGGGLVQPKGSLKLSCAASGFNFNTYAMNWVRQAPGKGLEWVARIRTKSNNFATYYAHSVKDRFTISRDDSESMLYLQMNNLKTEDTAMYYCVRQGLAYYAMDYWGQGTSVTVSS

SEQ ID NO: 141 - protein, 5 aa, Artificial Sequence
  ACI-7079-2501G2-Ab2 2501G2E5 VH CDR1
TYAMN

SEQ ID NO: 142 - protein, 19 aa, Artificial Sequence
  ACI-7079-2501G2-Ab2 2501G2E5 VH CDR2
RIRTKSNNFATYYAHSVKD

SEQ ID NO: 143 - protein, 10 aa, Artificial Sequence
  ACI-7079-2501G2-Ab2 2501G2E5 VH CDR3
QGLAYYAMDY

SEQ ID NO: 144 - protein, 112 aa, Artificial Sequence
  ACI-7079-2501G2-Ab2 2501G2E5 VL
DVLMTQTPLSLPVSLGDQVSISCRSSQTIVHSNGNTYLEWYLQKPGQSPKLLIYKVSNRFSGVPDRFSGSGSGTDFTLKISRVEAEDLGVYYCFQGSQGPLTFGAGTKLELK

SEQ ID NO: 145 - protein, 16 aa, Artificial Sequence
  ACI-7079-2501G2-Ab2 2501G2E5 VL CDR1
RSSQTIVHSNGNTYLE

SEQ ID NO: 146 - protein, 30 aa, Unknown
  Binding Epitope from an organism of the kingdom Mammalia
GKTKEGVLYVGSKTKEGVVHGVATVAEKTK

SEQ ID NO: 147 - protein, 45 aa, Unknown
  Binding Epitope from an organism of the kingdom Mammalia
KKDQLGKNEEGAPQEGILEDMPVDPDNEAYEMPSEEGYQDYEPEA

SEQ ID NO: 148 - DNA, 363 bp, Artificial Sequence
  ACI-7079-2501G2-Ab2 2501G2E5 VH
gaggtgcagcttgttgagtctggtggaggattggtgcagcctaaagggtcattgaaactctcatgtgcagcctctggattcaacttcaatacctatgccatgaactgggtccgccaggctccaggaaagggtttggaatgggttgctcgcataagaactaaaagtaataattttgcaacatattatgcccattcagtgaaagacagattcaccatctccagagatgattcagaaagcatgctctatctgcaaatgaacaacttgaaaactgaggacacagccatgtattactgtgtgagacagggactagcctactatgctatggactactggggtcaaggaacctcagtcaccgtctcctca

SEQ ID NO: 149 - DNA, 336 bp, Artificial Sequence
  ACI-7079-2501G2-Ab2 2501G2E5 VL
gatgttttgatgacccaaactccactctccctgcctgtcagtcttggagatcaagtctccatctcttgcagatctagtcaaaccattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacaaggtccgctcacgttcggtgctgggaccaaactggagctgaaa

SEQ ID NO: 150 - protein, 113 aa, Artificial Sequence
  ACI-7079-2503C6-Ab1 2503C6H9 VH
DVQLQESGPGLVKPSQSLSLTCSVTGYSITSGYYWNWIRLFPGNKLEWLGYINYDGSNNFNPSLKNRISITRDTSKNQFFLKLNSVTSEDTATYFCLRGDWDWGQGTLVTVSA

SEQ ID NO: 151 - protein, 6 aa, Artificial Sequence
  ACI-7079-2503C6-Ab1 2503C6H9 VH CDR1
SGYYWN

SEQ ID NO: 152 - protein, 16 aa, Artificial Sequence
  ACI-7079-2503C6-Ab1 2503C6H9 VH CDR2
YINYDGSNNFNPSLKN

SEQ ID NO: 153 - protein, 4 aa, Artificial Sequence
  ACI-7079-2503C6-Ab1 2503C6H9 VH CDR3
GDWD

SEQ ID NO: 154 - protein, 112 aa, Artificial Sequence
  ACI-7079-2503C6-Ab1 2503C6H9 VL
DVVMTQTPLTLSVTIGQPASISCKSSQSLLDSDGETYLNWLLQRPGQSPKRLICLVSKLDSGVPDRFTGSGSGTDFTLKISRVEAEDLGVYYCWQGTHFPQTFGGGTRLEIK

SEQ ID NO: 155 - protein, 16 aa, Artificial Sequence
  ACI-7079-2503C6-Ab1 2503C6H9 VL CDR1
KSSQSLLDSDGETYLN

SEQ ID NO: 156 - protein, 7 aa, Artificial Sequence
  ACI-7079-2503C6-Ab1 2503C6H9 VL CDR2
LVSKLDS

SEQ ID NO: 157 - protein, 9 aa, Artificial Sequence
  ACI-7079-2503C6-Ab1 2503C6H9 VL CDR3
WQGTHFPQT

SEQ ID NO: 158 - DNA, 339 bp, Artificial Sequence
  ACI-7079-2503C6-Ab1 2503C6H9 VH
gatgtacagcttcaggagtcaggacctggcctcgtgaaaccttctcagtctctgtctctcacctgctctgtcactggctactccatcaccagtggttattactggaactggatccgactatttccaggaaacaaactggaatggctgggctacataaactacgatggtagcaataacttcaacccatctctcaaaaatcgaatctccatcactcgtgacacatctaagaaccagtttttcctgaaattgaattctgtgacttctgaggacacagccacatatttctgtttaagaggggactgggactggggccaagggactctggtcactgtctctgca

SEQ ID NO: 159 - DNA, 336 bp, Artificial Sequence
  ACI-7079-2503C6-Ab1 2503C6H9 VL
gatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctgtctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaggctggaaatcaaa

SEQ ID NO: 160 - protein, 114 aa, Artificial Sequence
  ACI-7079-2504A6-Ab1 2504A6C8 VH
QVQLQQSGVELARPGASVKLSCKASGYTFTSYGISWVKQRTGQGLKWIGEIYPGSGNTYYNEKFKGKATLTADKSSSTAYMELRSLTSEDSAVYFCATDYDAYWGQGTTLTVSS

SEQ ID NO: 161 - protein, 5 aa, Artificial Sequence
  ACI-7079-2504A6-Ab1 2504A6C8 VH CDR1
SYGIS

SEQ ID NO: 162 - protein, 17 aa, Artificial Sequence
  ACI-7079-2504A6-Ab1 2504A6C8 VH CDR2
EIYPGSGNTYYNEKFKG

SEQ ID NO: 163 - protein, 5 aa, Artificial Sequence
  ACI-7079-2504A6-Ab1 2504A6C8 VH CDR3
DYDAY

SEQ ID NO: 164 - protein, 112 aa, Artificial Sequence
  ACI-7079-2504A6-Ab1 2504A6C8 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 165 - protein, 16 aa, Artificial Sequence
  ACI-7079-2504A6-Ab1 2504A6C8 VL CDR1
RSSQSLVHSNGNTYLH

SEQ ID NO: 166 - protein, 7 aa, Artificial Sequence
  ACI-7079-2504A6-Ab1 2504A6C8 VL CDR2
KVSNRFS

SEQ ID NO: 167 - protein, 9 aa, Artificial Sequence
  ACI-7079-2504A6-Ab1 2504A6C8 VL CDR3
SQSTHVPLT

SEQ ID NO: 168 - DNA, 342 bp, Artificial Sequence
  ACI-7079-2504A6-Ab1 2504A6C8 VH
caggttcagctgcagcagtctggagttgagctggcgaggcctggggcttcagtgaaactgtcctgcaaggcttctggctacaccttcacaagctatggtataagctgggtgaagcagagaactggacagggccttaagtggattggagagatttatcctggaagtggtaatacttactacaatgagaagttcaagggcaaggccacactgactgcagacaaatcctccagcacagcgtacatggagctccgcagcctgacgtctgaggactctgcggtctatttctgtgcaaccgattacgacgcctactggggccaaggcaccactctcacagtctcctca

SEQ ID NO: 169 - DNA, 336 bp, Artificial Sequence
  ACI-7079-2504A6-Ab1 2504A6C8 VL
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaa

SEQ ID NO: 170 - protein, 120 aa, Artificial Sequence
  ACI-7079-2506E2-Ab2 2506E2G4 VH
QVQLQQSGPELVRPGASVKISCKASGYAFSNSWMNWVKQRPGKGLEWIGRIFPGDGDTYYDGKFKGKVKLTTDKFSNTAYMQLRSLTSEDSAVYFCARWGGTNDEWFAHWGQGTLVTVSV

SEQ ID NO: 171 - protein, 5 aa, Artificial Sequence
  ACI-7079-2506E2-Ab2 2506E2G4 VH CDR1
NSWMN

SEQ ID NO: 172 - protein, 17 aa, Artificial Sequence
  ACI-7079-2506E2-Ab2 2506E2G4 VH CDR2
RIFPGDGDTYYDGKFKG

SEQ ID NO: 173 - protein, 11 aa, Artificial Sequence
  ACI-7079-2506E2-Ab2 2506E2G4 VH CDR3
WGGTNDEWFAH

SEQ ID NO: 174 - protein, 111 aa, Artificial Sequence
  ACI-7079-2506E2-Ab2 2506E2G4 VL
DIVLTQSPASLTVSLGQRATISCRASQSVSTSRNSYMHWYQQKPRQPPKLLIKYASNLESGVPARFSGSGSGADFTLNIHPVEEEDTATYYCQHSWDIPLTFGTGTKLELS

SEQ ID NO: 175 - protein, 15 aa, Artificial Sequence
  ACI-7079-2506E2-Ab2 2506E2G4 VL CDR1
RASQSVSTSRNSYMH

SEQ ID NO: 176 - protein, 7 aa, Artificial Sequence
  ACI-7079-2506E2-Ab2 2506E2G4 VL CDR2
YASNLES

SEQ ID NO: 177 - protein, 9 aa, Artificial Sequence
  ACI-7079-2506E2-Ab2 2506E2G4 VL CDR3
QHSWDIPLT

SEQ ID NO: 178 - DNA, 360 bp, Artificial Sequence
  ACI-7079-2506E2-Ab2 2506E2G4 VH
caggttcagttgcagcagtctggacctgagctggtgaggcctggggcctcagtgaagatttcctgcaaggcttctggctacgcattcagtaactcctggatgaactgggtgaagcagaggcctggaaagggtcttgagtggattggacggatttttcctggagatggagatacttactacgatgggaagttcaagggcaaggtcaaactgacaacagacaaattctccaacacagcctacatgcaactccgcagcctgacatctgaggactctgcggtctacttctgtgcaagatgggggggtactaacgatgagtggtttgctcactggggccaagggactctggtcactgtctctgta

SEQ ID NO: 179 - DNA, 333 bp, Artificial Sequence
  ACI-7079-2506E2-Ab2 2506E2G4 VL
gacattgtgctgacacagtctcctgcttccttaactgtatctctggggcagagggccaccatctcatgcagggccagccaaagtgtcagtacatctaggaatagttatatgcactggtaccaacagaaaccaagacagccacccaaactcctcatcaagtatgcatccaacctagaatctggggtccctgccaggttcagtggcagtgggtctggggcagacttcaccctcaacatccatcctgtggaggaggaggatactgcaacatattactgtcagcacagttgggatattccgctcacgttcggtactgggaccaagctggagctgagt

SEQ ID NO: 180 - protein, 114 aa, Artificial Sequence
  ACI-7079-2506F3-Ab1 2506F3E12 VH
QVQLQQPGAELVKPGASVKLSCKASGYTFTTYWMQWVKQRPGQGLEWIGEIDPSDSYINYNQKFKGKATLTVDTSSSTAFMQLSSLTSEDSAVYYCARGMMDYWGQGTSVTVSS

SEQ ID NO: 181 - protein, 5 aa, Artificial Sequence
  ACI-7079-2506F3-Ab1 2506F3E12 VH CDR1
TYWMQ

SEQ ID NO: 182 - protein, 17 aa, Artificial Sequence
  ACI-7079-2506F3-Ab1 2506F3E12 VH CDR2
EIDPSDSYINYNQKFKG

SEQ ID NO: 183 - protein, 5 aa, Artificial Sequence
  ACI-7079-2506F3-Ab1 2506F3E12 VH CDR3
GMMDY

SEQ ID NO: 184 - protein, 112 aa, Artificial Sequence
  ACI-7079-2506F3-Ab1 2506F3E12 VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFKGSHVPYTFGGGTKLEIK

SEQ ID NO: 185 - protein, 16 aa, Artificial Sequence
  ACI-7079-2506F3-Ab1 2506F3E12 VL CDR1
RSSQSIVHSNGNTYLE

SEQ ID NO: 186 - protein, 7 aa, Artificial Sequence
  ACI-7079-2506F3-Ab1 2506F3E12 VL CDR2
KVSNRFS

SEQ ID NO: 187 - protein, 9 aa, Artificial Sequence
  ACI-7079-2506F3-Ab1 2506F3E12 VL CDR3
FKGSHVPYT

SEQ ID NO: 188 - DNA, 342 bp, Artificial Sequence
  ACI-7079-2506F3-Ab1 2506F3E12 VH
caggtccaactgcagcagcctggggctgagcttgtgaagcctggggcttcagtgaagctgtcctgcaaggcttctggctacaccttcaccacctactggatgcagtgggtaaaacagaggcctggacagggccttgagtggatcggagagattgatccttctgatagctatattaactacaatcaaaagttcaagggcaaggccacattgactgtagacacatcctccagcacagccttcatgcagctcagcagcctgacatctgaggactctgcggtctattactgtgcaagggggatgatggactactggggtcaaggaacctcagtcaccgtctcctca

SEQ ID NO: 189 - DNA, 336 bp, Artificial Sequence
  ACI-7079-2506F3-Ab1 2506F3E12 VL
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttaaaggttcacatgttccgtacacgttcggaggggggaccaagctggaaataaaa

SEQ ID NO: 190 - protein, 120 aa, Artificial Sequence
  ACI-7079-2507B3-Ab1 2507B3G8 VH
EVQLVESGGGLVQPKGSLKVSCAASGFTFKTYAMHWVRQAPGKGLEWVARIRSENSNFAKYYADSVKDRFTISRDDSQSMLYLQMHTLKTEDTAIYYCVRGYNGSSLDYWGQGTTLTVSS

SEQ ID NO: 191 - protein, 5 aa, Artificial Sequence
  ACI-7079-2507B3-Ab1 2507B3G8 VH CDR1
TYAMH

SEQ ID NO: 192 - protein, 19 aa, Artificial Sequence
  ACI-7079-2507B3-Ab1 2507B3G8 VH CDR2
RIRSENSNFAKYYADSVKD

SEQ ID NO: 193 - protein, 9 aa, Artificial Sequence
  ACI-7079-2507B3-Ab1 2507B3G8 VH CDR3
GYNGSSLDY

SEQ ID NO: 194 - protein, 106 aa, Artificial Sequence
  ACI-7079-2507B3-Ab1 2507B3G8 VL
QIVLTQSPAIMSAFPGERVTMTCSASSSVNYMHWYQQKSGTSPKRWIYDTSKLASGVPARFSGGGSGTSYSLTISNMEAEDAATYYCQQWRSNPPTFGGGTKLEIK

SEQ ID NO: 195 - protein, 10 aa, Artificial Sequence
  ACI-7079-2507B3-Ab1 2507B3G8 VL CDR1
SASSSVNYMH

SEQ ID NO: 196 - protein, 7 aa, Artificial Sequence
  ACI-7079-2507B3-Ab1 2507B3G8 VL CDR2
DTSKLAS

SEQ ID NO: 197 - protein, 9 aa, Artificial Sequence
  ACI-7079-2507B3-Ab1 2507B3G8 VL CDR3
QQWRSNPPT

SEQ ID NO: 198 - DNA, 360 bp, Artificial Sequence
  ACI-7079-2507B3-Ab1 2507B3G8 VH
gaggtgcagcttgttgagtctggtggaggattggtgcagcctaaaggatcattgaaagtctcatgtgccgcctctggtttcaccttcaagacctatgccatgcactgggtccgccaggctccgggaaagggtttggaatgggttgctcgcataagaagtgaaaacagtaattttgcaaaatattatgccgattcagtgaaagacagattcaccatctccagagatgattcacaaagtatgctctatctgcaaatgcacaccctgaaaactgaggacacagccatctattattgtgtaaggggatataacggcagtagccttgactactggggccaaggcaccactctcacagtctcctca

SEQ ID NO: 199 - DNA, 318 bp, Artificial Sequence
  ACI-7079-2507B3-Ab1 2507B3G8 VL
caaattgttctcacccagtctccagcaatcatgtctgcatttccaggggagagggtcaccatgacctgcagtgccagctcaagtgtaaattacatgcactggtaccagcagaagtccggcacctcccccaaaagatggatttatgacacatccaaactggcttctggagtccctgctcgcttcagtggcggtgggtctgggacctcttactctctcacaatcagcaacatggaggctgaagatgctgccacttattactgccagcagtggagaagtaatccacccactttcggaggggggaccaagctggaaataaaa

SEQ ID NO: 200 - protein, 113 aa, Artificial Sequence
  ACI-7079-2511B3-Ab3 2511B3B12 VH
DVQLQESGPGLVKPSQSLSLTCSVTGFSITSYYYWNWIRQFPGNKLEWMAYISYDGSNNYNPSLKNRISITRDTSKNQFFLKLNSVTTEDTATYYCTRGDWDWGQGTLVTVSA

SEQ ID NO: 201 - protein, 6 aa, Artificial Sequence
  ACI-7079-2511B3-Ab3 2511B3B12 VH CDR1
SYYYWN

SEQ ID NO: 202 - protein, 16 aa, Artificial Sequence
  ACI-7079-2511B3-Ab3 2511B3B12 VH CDR2
YISYDGSNNYNPSLKN

SEQ ID NO: 203 - protein, 4 aa, Artificial Sequence
  ACI-7079-2511B3-Ab3 2511B3B12 VH CDR3
GDWD

SEQ ID NO: 204 - protein, 112 aa, Artificial Sequence
  ACI-7079-2511B3-Ab3 2511B3B12 VL
DVVMTQTPLTLSLTIGQPASISCKSSQSLLDSDGETYLNWLLQRPGQSPKRLIYLVSKLESGVPDRFTGSGSGTVFTLKISRVEAEDLGVYYCWQGTHFPQTFGGGTKLEIK

SEQ ID NO: 205 - protein, 16 aa, Artificial Sequence
  ACI-7079-2511B3-Ab3 2511B3B12 VL CDR1
KSSQSLLDSDGETYLN

SEQ ID NO: 206 - protein, 7 aa, Artificial Sequence
  ACI-7079-2511B3-Ab3 2511B3B12 VL CDR2
LVSKLES

SEQ ID NO: 207 - protein, 9 aa, Artificial Sequence
  ACI-7079-2511B3-Ab3 2511B3B12 VL CDR3
WQGTHFPQT

SEQ ID NO: 208 - DNA, 339 bp, Artificial Sequence
  ACI-7079-2511B3-Ab3- 2511B3B12 VH
gatgtacagcttcaggaatcaggacctggcctcgtgaaaccttctcagtctctgtctctcacctgctctgtcactggcttctccatcaccagttattattactggaactggatccggcagtttccaggaaacaaactggaatggatggcctacataagctacgatggtagcaataactacaacccatctctcaaaaatcgaatctccatcactcgtgacacatctaagaaccagtttttcctgaagttgaattctgtgactactgaggacacagccacatattactgtacaagaggggactgggactggggccaagggactctggtcactgtctctgca

SEQ ID NO: 209 - DNA, 336 bp, Artificial Sequence
  ACI-7079-2511B3-Ab3- 2511B3B12 VL
gatgttgtgatgacccagactccactcactttgtcgcttaccattggacaaccagcctccatctcttgcaagtcaagtcagagcctcttagatagtgatggagagacatatttgaattggttgttacagaggccaggtcagtctccaaagcgcctaatctatctggtgtctaaactggaatctggagtccctgacaggttcactggcagtggatcagggacagttttcacactgaaaatcagcagagtggaggctgaggatttgggagtttattattgctggcaagggacacattttcctcagacgttcggtggaggcaccaagctggaaatcaaa

SEQ ID NO: 210 - protein, 113 aa, Artificial Sequence
  ACI-7079-2601B6-Ab1 2601B6D2 VH
EIQLQQSGAELVRPGASVKLSCTTSGFNIKDDYIHWVKQRPEQGLEWIGWIDPENGDTDYASKFQGKATITADTSSNTAYLHLSSLTSEDAAVYFCTTRGFGYWGQGTLVTVS

SEQ ID NO: 211 - protein, 5 aa, Artificial Sequence
  ACI-7079-2601B6-Ab1 2601B6D2 VH CDR1
DDYIH

SEQ ID NO: 212 - protein, 17 aa, Artificial Sequence
  ACI-7079-2601B6-Ab1 2601B6D2 VH CDR2
WIDPENGDTDYASKFQG

SEQ ID NO: 213 - protein, 5 aa, Artificial Sequence
  ACI-7079-2601B6-Ab1 2601B6D2 VH CDR3
RGFGY

SEQ ID NO: 214 - protein, 107 aa, Artificial Sequence
  ACI-7079-2601B6-Ab1 2601B6D2 VL
DIVMTQSHKFMSTSVGDRVSITCKASQDVGNVVAWYQQKPGQSPKLLIYWASSRHTGVPDRFTGSGSGTEFTLTISNVQSEDLADYFCQQYSSYPLTFGAGTKLELK

SEQ ID NO: 215 - protein, 11 aa, Artificial Sequence
  ACI-7079-2601B6-Ab1 2601B6D2 VL CDR1
KASQDVGNVVA

SEQ ID NO: 216 - protein, 7 aa, Artificial Sequence
  ACI-7079-2601B6-Ab1 2601B6D2 VL CDR2
WASSRHT

SEQ ID NO: 217 - protein, 9 aa, Artificial Sequence
  ACI-7079-2601B6-Ab1 2601B6D2 VL CDR3
QQYSSYPLT

SEQ ID NO: 218 - DNA, 339 bp, Artificial Sequence
  ACI-7079-2601B6-Ab1 2601B6D2 VH
gagattcaactgcagcagtctggggctgagcttgtgaggccaggggcctcagtcaagttgtcctgcacaacttccggctttaacattaaagacgactatattcactgggtgaagcagaggcctgaacagggcctggagtggattggatggattgatcctgagaatggtgatactgattatgcctcgaagttccagggcaaggccactataacagcagacacatcctccaacacagcctacctgcacctcagcagcctgacatcagaggacgctgccgtctatttctgtactacaagaggatttggttactggggccaagggactctggtcactgtctct

SEQ ID NO: 219 - DNA, 321 bp, Artificial Sequence
  ACI-7079-2601B6-Ab1 2601B6D2 VL
gacattgtgatgacccagtctcacaaattcatgtccacatcagtaggagacagggtcagcatcacctgcaaggccagtcaggatgtgggtaatgttgttgcctggtatcaacagaaaccaggacaatctcctaaactactgatttactgggcatcctcccggcacactggagtccctgatcgcttcacaggcagtggatctgggacagaattcactctcaccattagcaatgtgcagtctgaagacttggcagattatttctgtcagcaatatagcagctatccgctcacgttcggtgctgggaccaagctggagctgaag

SEQ ID NO: 220 - protein, 120 aa, Artificial Sequence
  ACI-7079-2602G4-Ab4 2602G4H1 VH
EVQLVESGGGLVQPKGSLKLSCAASGFTFKTYAMHWVRQAPGKGLEWVARIRSKGSDYATYYADSVKDRFTISRDDSQSMLYLQMNNLKTEDTAMYFCVRGGADSWFAYWGQGTLVTVST

SEQ ID NO: 221 - protein, 5 aa, Artificial Sequence
  ACI-7079-2602G4-Ab4 2602G4H1 VH CDR1
TYAMH

SEQ ID NO: 222 - protein, 19 aa, Artificial Sequence
  ACI-7079-2602G4-Ab4 2602G4H1 VH CDR2
RIRSKGSDYATYYADSVKD

SEQ ID NO: 223 - protein, 9 aa, Artificial Sequence
  ACI-7079-2602G4-Ab4 2602G4H1 VH CDR3
GGADSWFAY

SEQ ID NO: 224 - protein, 106 aa, Artificial Sequence
  ACI-7079-2602G4-Ab4 2602G4H1 VL
QIVLTQSPAIMSASPGERITMTCTASSSVSYMHWYQQKSGTSPKRWIYDTSKLASGVPARFSGSGSGASYTLTISSMEAEDAATYYCQQWNRNPPTFGGGTQLAIK

SEQ ID NO: 225 - protein, 10 aa, Artificial Sequence
  ACI-7079-2602G4-Ab4 2602G4H1 VL CDR1
TASSSVSYMH

SEQ ID NO: 226 - protein, 7 aa, Artificial Sequence
  ACI-7079-2602G4-Ab4 2602G4H1 VL CDR2
DTSKLAS

SEQ ID NO: 227 - protein, 9 aa, Artificial Sequence
  ACI-7079-2602G4-Ab4 2602G4H1 VL CDR3
QQWNRNPPT

SEQ ID NO: 228 - DNA, 360 bp, Artificial Sequence
  ACI-7079-2602G4-Ab4 2602G4H1 VH
gaggtgcagcttgttgagtctggtggaggattggtgcagcctaaaggatcattgaaactctcatgtgccgcctctggtttcaccttcaagacctatgccatgcactgggtccgccaggctccaggaaagggtttggaatgggttgctcgcataagaagtaaaggtagtgattatgcaacatattatgccgattcagtgaaggacagattcaccatctccagagatgattcacaaagcatgctctatctgcaaatgaacaacctgaaaactgaggatacagccatgtatttctgtgtgagagggggtgctgactcctggtttgcttactggggccaagggactctggtcactgtctctaca

SEQ ID NO: 229 - DNA, 318 bp, Artificial Sequence
  ACI-7079-2602G4-Ab4 2602G4H1 VL
caaattgttctcacccagtctccagcaatcatgtctgcatctccaggggagaggatcaccatgacctgcactgccagctcaagtgtaagttacatgcactggtaccagcagaagtcaggcacctcccccaaaagatggatttatgacacatccaaactggcttctggagtccctgctcgcttcagtggcagtgggtctggggcctcttatactctcacaatcagcagcatggaggctgaagatgctgccacttattactgccagcagtggaatcgtaacccaccgacgttcggtggaggcacccagctggcaatcaaa

SEQ ID NO: 230 - protein, 114 aa, Artificial Sequence
  ACI-7079-2603C1-Ab3 2603C1H6 VH
QVQLQQPGADLVKPGASVKLSCKASGYTFTSYWMQWTKQRPGQGLEWIGEIDPSDSYANYNQKFKGKATLTVDKYSSTAYMQLNSLTSEDSAVYYCALYDGPSYWGQGTLVTVS

SEQ ID NO: 231 - protein, 5 aa, Artificial Sequence
  ACI-7079-2603C1-Ab3 2603C1H6 VH CDR1
SYWMQ

SEQ ID NO: 232 - protein, 17 aa, Artificial Sequence
  ACI-7079-2603C1-Ab3 2603C1H6 VH CDR2
EIDPSDSYANYNQKFKG

SEQ ID NO: 233 - protein, 6 aa, Artificial Sequence
  ACI-7079-2603C1-Ab3 2603C1H6 VH CDR3
YDGPSY

SEQ ID NO: 234 - protein, 106 aa, Artificial Sequence
  ACI-7079-2603C1-Ab3 2603C1H6 VL
ENVLTQSPAIMSASPGEKVTMTCSAGSSVSYMHWFQQKSSTSPKLWIYDTSKLPSGVPGRFSGSGSGNSYSLTISSMEAEDVATYYCFQGSGYPYTFGGGTKLEIK

SEQ ID NO: 235 - protein, 10 aa, Artificial Sequence
  ACI-7079-2603C1-Ab3 2603C1H6 VL CDR1
SAGSSVSYMH

SEQ ID NO: 236 - protein, 7 aa, Artificial Sequence
  ACI-7079-2603C1-Ab3 2603C1H6 VL CDR2
DTSKLPS

SEQ ID NO: 237 - protein, 9 aa, Artificial Sequence
  ACI-7079-2603C1-Ab3 2603C1H6 VL CDR3
FQGSGYPYT

SEQ ID NO: 238 - DNA, 342 bp, Artificial Sequence
  ACI-7079-2603C1-Ab3 2603C1H6 VH
caggtccaactgcagcaacctggggctgaccttgtgaagcctggggcttcagtgaagctgtcctgtaaggcttctggctacaccttcaccagttactggatgcagtggacaaaacagaggcctggacagggccttgagtggatcggagagattgatccttctgatagctatgctaactacaatcaaaagttcaagggcaaggccacattgactgttgacaaatattccagcacagcctacatgcagctcaacagcctgacatctgaggactctgcggtctattactgtgccctctatgatggtccctcttactggggccaagggactctggtcactgtctct

SEQ ID NO: 239 - DNA, 318 bp, Artificial Sequence
  ACI-7079-2603C1-Ab3 2603C1H6 VL
gaaaatgttctcacccagtctccagcaatcatgtctgcatctccaggggaaaaggtcaccatgacctgcagtgccggctcaagtgtaagttacatgcactggttccaacagaagtcaagcacctcccccaaactctggatttatgacacatccaaactgccttctggagtcccaggtcgcttcagtggcagtgggtctggaaactcttactctctcacgatcagcagcatggaggctgaagatgttgccacttattactgttttcaggggagtgggtacccgtacacgttcggaggggggaccaagctggaaataaaa

SEQ ID NO: 240 - protein, 120 aa, Artificial Sequence
  ACI-7079-2603F3-Ab1 2603F3H3 VH
EVQLVESGGGLVQPKGSLKLSCAASGFTFNTYAMHWVRQAPGKGLEWVARIRSKGSNYATYYADSVKDRFTISRDDSQSMLYLQMNNLKTEDTAMYYCVRGGGDSWFAYWGQGTLVTVSA

SEQ ID NO: 241 - protein, 5 aa, Artificial Sequence
  ACI-7079-2603F3-Ab1 2603F3H3 VH CDR1
TYAMH

SEQ ID NO: 242 - protein, 19 aa, Artificial Sequence
  ACI-7079-2603F3-Ab1 2603F3H3 VH CDR2
RIRSKGSNYATYYADSVKD

SEQ ID NO: 243 - protein, 9 aa, Artificial Sequence
  ACI-7079-2603F3-Ab1 2603F3H3 VH CDR3
GGGDSWFAY

SEQ ID NO: 244 - protein, 106 aa, Artificial Sequence
  ACI-7079-2603F3-Ab1 2603F3H3 VL
QIVLTQSPAIMSASPGERVTMTCTASSSVSYMHWYQQKSGTSPKRWIYDTSKLASGVPARFSGSGSGASYTLTISSMEAEDAATYYCQQWNSNPPTFGGGTQLAIK

SEQ ID NO: 245 - protein, 10 aa, Artificial Sequence
  ACI-7079-2603F3-Ab1 2603F3H3 VL CDR1
TASSSVSYMH

SEQ ID NO: 246 - protein, 7 aa, Artificial Sequence
  ACI-7079-2603F3-Ab1 2603F3H3 VL CDR2
DTSKLAS

SEQ ID NO: 247 - protein, 9 aa, Artificial Sequence
  ACI-7079-2603F3-Ab1 2603F3H3 VL CDR3
QQWNSNPPT

SEQ ID NO: 248 - DNA, 360 bp, Artificial Sequence
  ACI-7079-2603F3-Ab1 2603F3H3 VH
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaggtagtaattatgcaacatattatgccgattcagtgaaagacagattcaccatctccagagatgattcacaaagcatgctctatctgcaaatgaacaacctgaaaactgaggacacagccatgtattactgtgtgagagggggtggtgactcctggtttgcttactggggccaagggactctggtcactgtctctgca

SEQ ID NO: 249 - DNA, 318 bp, Artificial Sequence
  ACI-7079-2603F3-Ab1 2603F3H3 VL
caaattgttctcacccagtctccagcaatcatgtctgcatctccaggggagagggtcaccatgacctgcactgccagctcaagtgtaagttacatgcactggtaccagcagaagtcaggcacctcccccaaaagatggatttatgacacatccaaactggcttctggagtccctgctcgcttcagtggcagtgggtctggggcctcttatactctcacaatcagcagcatggaggctgaagatgctgccacttattactgccagcagtggaatagtaacccaccgacgttcggtggaggcacccagctggcaatcaaa

SEQ ID NO: 250 - protein, 120 aa, Artificial Sequence
  ACI-7079-2605B3-Ab2 2605B3D1 VH
EVQLVESGGGLVQPKGSLKLSCAASGFIFKTYAMHWVRQAPGKGLEWVARIRSKGGNYATYFADSVKDRFTISRDDSQNMLYLQVNNLKIEDTAMYFCVRGGNYSWFAYWGQGTLVTVSA

SEQ ID NO: 251 - protein, 5 aa, Artificial Sequence
  ACI-7079-2605B3-Ab2 2605B3D1 VH CDR1
TYAMH

SEQ ID NO: 252 - protein, 19 aa, Artificial Sequence
  ACI-7079-2605B3-Ab2 2605B3D1 VH CDR2
RIRSKGGNYATYFADSVKD

SEQ ID NO: 253 - protein, 9 aa, Artificial Sequence
  ACI-7079-2605B3-Ab2 2605B3D1 VH CDR3
GGNYSWFAY

SEQ ID NO: 254 - protein, 106 aa, Artificial Sequence
  ACI-7079-2605B3-Ab2 2605B3D1 VL
QIVLTQSPAIMSASPGEKVTMTCSASSSVTYMHWYQQKSGTSPKRWIYDTSQLASGVPARFSGSGSGTSHSLTISSMETEDAATYYCQQWTRNPPTFGGGTKLAIK

SEQ ID NO: 255 - protein, 10 aa, Artificial Sequence
  ACI-7079-2605B3-Ab2 2605B3D1 VL CDR1
SASSSVTYMH

SEQ ID NO: 256 - protein, 7 aa, Artificial Sequence
  ACI-7079-2605B3-Ab2 2605B3D1 VL CDR2
DTSQLAS

SEQ ID NO: 257 - protein, 8 aa, Artificial Sequence
  ACI-7079-2605B3-Ab2 2605B3D1 VL CDR3
QQWTRNPP

SEQ ID NO: 258 - DNA, 360 bp, Unknown
  ACI-7079-2605B3-Ab2 2605B3D1 VH from an organism of the kingdom Mammalia
gaggtgcagcttgttgagtctggtggaggattggtgcagcctaaaggatcattgaaactctcatgtgccgcctctggtttcatctttaaaacctatgccatgcattgggtccgccaggctccaggaaagggtttggaatgggttgctcgaataagaagtaaaggtggtaattatgcaacatattttgccgattcagtgaaagacagattcaccatctccagagatgattcacaaaatatgctctatctgcaagtgaacaacctgaaaattgaggacacagccatgtatttctgtgtgagagggggtaattactcctggtttgcttactggggccaagggactctggtcactgtctctgca

SEQ ID NO: 259 - DNA, 318 bp, Unknown
  ACI-7079-2605B3-Ab2 2605B3D1 VL from an organism of the kingdom Mammalia
caaattgttctcacccagtctccagcaatcatgtctgcttctccaggggagaaggtcaccatgacctgcagtgccagctcaagtgtaacttacatgcattggtaccagcagaagtcaggcacctcccccaaaagatggatttatgacacatcccaactggcttctggagtccctgctcgcttcagtggcagtgggtctgggacctctcactctctcacaatcagcagcatggagactgaagatgctgccacttattactgccaacaatggactagaaacccaccgacgttcggtggaggcaccaagctggcaatcaaa

SEQ ID NO: 260 - protein, 119 aa, Artificial Sequence
  ACI-7079-2606A6-Ab2 2606A6D5 VH
QVQLQQSGPELVKPGASVKISCKASGFAFSSSWMNWVKQRPGKGLEWVGRIFPGDGDTNYDRKFKDKATLTADKSSSTAYMQLSSLTSEDSAVYFCARWTGGYDWFAYWGQGTLVTVSA

SEQ ID NO: 261 - protein, 5 aa, Artificial Sequence
  ACI-7079-2606A6-Ab2 2606A6D5 VH CDR1
SSWMN

SEQ ID NO: 262 - protein, 17 aa, Artificial Sequence
  ACI-7079-2606A6-Ab2 2606A6D5 VH CDR2
RIFPGDGDTNYDRKFKD

SEQ ID NO: 263 - protein, 10 aa, Artificial Sequence
  ACI-7079-2606A6-Ab2 2606A6D5 VH CDR3
WTGGYDWFAY

SEQ ID NO: 264 - protein, 111 aa, Artificial Sequence
  ACI-7079-2606A6-Ab2 2606A6D5 VL
DIVLTQSPASLAVSLGQRATISCRASQSVSTSNYNYLHWYQQKPGQPPKLLITYASNLESGVPARFSGSGSGTDFTLNIHPVEEGDTATYYCQHSWEIPLTFGAGTKLELK

SEQ ID NO: 265 - protein, 15 aa, Artificial Sequence
  ACI-7079-2606A6-Ab2 2606A6D5 VL CDR1
RASQSVSTSNYNYLH

SEQ ID NO: 266 - protein, 7 aa, Artificial Sequence
  ACI-7079-2606A6-Ab2 2606A6D5 VL CDR2
YASNLES

SEQ ID NO: 267 - protein, 9 aa, Artificial Sequence
  ACI-7079-2606A6-Ab2 2606A6D5 VL CDR3
QHSWEIPLT

SEQ ID NO: 268 - DNA, 357 bp, Artificial Sequence
  ACI-7079-2606A6-Ab2 2606A6D5 VH
caggttcagctgcaacagtctggacctgagctggtgaagcctggggcctcagtgaagatttcctgcaaggcttctggcttcgcattcagtagctcctggatgaactgggtgaagcagaggcctggaaagggtcttgagtgggttggacggatttttcctggagatggagatactaactacgataggaagttcaaggacaaggccacactgactgcagacaaatcctccagcacagcctacatgcaactcagcagcctgacatctgaggactctgcggtctacttctgtgcaagatggacggggggttacgactggtttgcttactggggccaagggactctggtcactgtctctgca

SEQ ID NO: 269 - DNA, 333 bp, Artificial Sequence
  ACI-7079-2606A6-Ab2 2606A6D5 VL
gacattgtgctgacacagtctcctgcttccttagctgtatctctggggcagagggccaccatctcatgcagggccagccaaagtgtcagtacatctaactataattatcttcactggtaccaacagaaaccaggacagccacccaaactcctcatcacgtatgcatccaacctagaatctggggtccctgccaggttcagtggcagtgggtctgggacagacttcaccctcaacatccatcctgtggaggagggagatactgcaacatattactgtcaacacagttgggagattccgctcacgttcggtgctgggaccaagctggagctgaaa

SEQ ID NO: 270 - protein, 117 aa, Artificial Sequence
  ACI-7079-2509E5-Ab2 2509E5E5 VH
EVQLQQSGPELVKPGASLKMSCKASGYSFTDYNMHWVKQSRGKSLEWIGYINPNNGVPTYKQKFKGRATLTVNQSSSTAYMEIRSLTSEDSAVYYCTRGGDHRFAYWGQGTLVTVSA

SEQ ID NO: 271 - protein, 5 aa, Artificial Sequence
  ACI-7079-2509E5-Ab2 2509E5E5 VH CDR1
DYNMH

SEQ ID NO: 272 - protein, 17 aa, Artificial Sequence
  ACI-7079-2509E5-Ab2 2509E5E5 VH CDR2
YINPNNGVPTYKQKFKG

SEQ ID NO: 273 - protein, 8 aa, Artificial Sequence
  ACI-7079-2509E5-Ab2 2509E5E5 VH CDR3
GGDHRFAY

SEQ ID NO: 274 - protein, 111 aa, Artificial Sequence
  ACI-7079-2509E5-Ab2 2509E5E5 VL
DIVLTQSPASLAVSLGQRATISCRASESVDYYGFSFVNWFQQKPGQPPKLLIYSASYKGSGVPVRFSGSGSGTDFSLSIHPMEADDTAMYFCQQNKEVPLTFGAGTKLELK

SEQ ID NO: 275 - protein, 15 aa, Artificial Sequence
  ACI-7079-2509E5-Ab2 2509E5E5 VL CDR1
RASESVDYYGFSFVN

SEQ ID NO: 276 - protein, 7 aa, Artificial Sequence
  ACI-7079-2509E5-Ab2 2509E5E5 VL CDR2
SASYKGS

SEQ ID NO: 277 - protein, 9 aa, Artificial Sequence
  ACI-7079-2509E5-Ab2 2509E5E5 VL CDR3
QQNKEVPLT

SEQ ID NO: 278 - DNA, 351 bp, Artificial Sequence
  ACI-7079-2509E5-Ab2 2509E5E5 VH
gaggtccagctgcaacagtctggacctgaactggtgaagcctggggcttcgctgaagatgtcctgcaaggcttctggatactcattcactgactacaacatgcactgggtgaaacagagccgtggaaagagccttgagtggattggatatattaaccctaacaatggtgttcccacgtataagcagaagttcaagggcagggccaccttgactgtaaaccagtcctccagcacagcctacatggagatccgcagcctgacatcggaagattctgcagtctattactgtacaagagggggtgatcaccggtttgcttactggggccaagggactctggtcactgtctctgca

SEQ ID NO: 279 - DNA, 333 bp, Artificial Sequence
  ACI-7079-2509E5-Ab2 2509E5E5 VL
gacattgtgctgacccaatctccagcttctttggctgtgtctctagggcagagggccaccatctcctgcagagccagcgaaagtgttgattattatggctttagttttgtgaactggttccaacagaaaccaggacagccacccaaactcctcatctatagtgcgtcctacaaaggatccggggtccctgtcaggttcagtggcagtgggtctgggacagacttcagtctcagcatccatcctatggaggcggatgatactgcaatgtatttctgtcagcaaaataaggaggttccgctcacgttcggtgctgggaccaagctggagctgaaa

SEQ ID NO: 280 - protein, 120 aa, Artificial Sequence
  ACI-7079-2501B11-Ab3 2501B11C7 VH
QVQLQQSGPELVKPGASVKISCKASGYAFSSFWMNWMKQRPGKGLEWIGRIYPGDGDAHYNGEFKGRATLTADKSSSTAYMQLSSLTSEDSAVYFCARKGDFYGSNYDYWGQGTTLTVSS

SEQ ID NO: 281 - protein, 4 aa, Artificial Sequence
  ACI-7079-2501B11-Ab3 2501B11C7 VH CDR1
SFWM

SEQ ID NO: 282 - protein, 17 aa, Artificial Sequence
  ACI-7079-2501B11-Ab3 2501B11C7 VH CDR2
RIYPGDGDAHYNGEFKG

SEQ ID NO: 283 - protein, 11 aa, Artificial Sequence
  ACI-7079-2501B11-Ab3 2501B11C7 VH CDR3
KGDFYGSNYDY

SEQ ID NO: 284 - protein, 109 aa, Artificial Sequence
  ACI-7079-2501B11-Ab3 2501B11C7 VL
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTRLTVL

SEQ ID NO: 285 - protein, 14 aa, Artificial Sequence
  ACI-7079-2501B11-Ab3 2501B11C7 VL CDR1
RSSTGAVTTSNYAN

SEQ ID NO: 286 - protein, 7 aa, Artificial Sequence
  ACI-7079-2501B11-Ab3 2501B11C7 VL CDR2
GTNNRAP

SEQ ID NO: 287 - protein, 9 aa, Artificial Sequence
  ACI-7079-2501B11-Ab3 2501B11C7 VL CDR3
ALWYSNHLV

SEQ ID NO: 288 - DNA, 360 bp, Artificial Sequence
  ACI-7079-2501B11-Ab3 2501B11C7 VH
caggttcagctgcagcagtctggacctgagctggtgaagcctggggcctcagtgaagatttcctgcaaggcttctggctacgcattcagtagtttctggatgaactggatgaaacagaggcctggaaagggtcttgagtggattggacggatttatcctggagatggagatgctcactacaatggggagttcaagggcagggccacactgactgcagacaaatcctccagcacagcctacatgcaactcagcagcctgacatctgaggactctgcggtctacttctgtgcaagaaagggggatttctacggtagtaactacgactattggggccaaggcaccactctcacagtctcctca

SEQ ID NO: 289 - DNA, 327 bp, Artificial Sequence
  ACI-7079-2501B11-Ab3 2501B11C7 VL
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccagactgactgtccta

SEQ ID NO: 290 - protein, 120 aa, Artificial Sequence
  ACI-7079-2501D10-Ab1 2501D10C3 VH
EVQLVESGGGLVQPKGSLKVSCAASGFTFKTYAMHWVRQAPGKGLEWVARIRSENSNFAKYYADSVKDRFTISRDDSQSMLYLQMNNLKTEDTAMYYCVRGYNGSSLDYWGQGTTLTVSS

SEQ ID NO: 291 - protein, 5 aa, Artificial Sequence
  ACI-7079-2501D10-Ab1 2501D10C3 VH CDR1
TYAMH

SEQ ID NO: 292 - protein, 19 aa, Artificial Sequence
  ACI-7079-2501D10-Ab1 2501D10C3 VH CDR2
RIRSENSNFAKYYADSVKD

SEQ ID NO: 293 - protein, 9 aa, Artificial Sequence
  ACI-7079-2501D10-Ab1 2501D10C3 VH CDR3
GYNGSSLDY

SEQ ID NO: 294 - protein, 106 aa, Artificial Sequence; identical to SEQ ID NO: 194
  ACI-7079-2501D10-Ab1 2501D10C3 VL

SEQ ID NO: 295 - protein, 10 aa, Artificial Sequence
  ACI-7079-2501D10-Ab1 2501D10C3 VL CDR1
SASSSVNYMH

SEQ ID NO: 296 - protein, 7 aa, Artificial Sequence
  ACI-7079-2501D10-Ab1 2501D10C3 VL CDR2
DTSKLAS

SEQ ID NO: 297 - protein, 9 aa, Artificial Sequence
  ACI-7079-2501D10-Ab1 2501D10C3 VL CDR3
QQWRSNPPT

SEQ ID NO: 298 - DNA, 360 bp, Artificial Sequence
  ACI-7079-2501D10-Ab1 2501D10C3 VH
gaggtgcagcttgttgagtctggtggaggattggtgcagcctaaaggatcattgaaagtctcatgtgccgcctctggtttcaccttcaagacctatgccatgcactgggtccgccaggctccgggaaagggtttggaatgggttgctcgcataagaagtgaaaacagtaattttgcaaaatattatgccgattcagtgaaggacagattcaccatctccagagatgattcacaaagtatgctctatctgcaaatgaacaacctgaaaactgaggacacagccatgtattattgtgtaaggggatataacggcagtagccttgactactggggccaaggcaccactctcacagtctcctca

SEQ ID NO: 299 - DNA, 318 bp, Artificial Sequence; identical to SEQ ID NO: 199
  ACI-7079-2501D10-Ab1 2501D10C3 VL

SEQ ID NO: 300 - protein, 117 aa, Artificial Sequence
  ACI-7087-4119E10-Ab2 VH
QVQLQQSGAELVRPGASVTLSCKASGYTFSDYEMNWVKQTPVHGLEWIGAIDPETGGTAYNQKFKGKAILTSDKSSSTAYMELRSLTSEDSAVYYCTRFLLIDFDYWGQGTTLTVSS

SEQ ID NO: 301 - protein, 5 aa, Artificial Sequence
  ACI-7087-4119E10-Ab2 VH CDR1
DYEMN

SEQ ID NO: 302 - protein, 17 aa, Artificial Sequence
  ACI-7087-4119E10-Ab2 VH CDR2
AIDPETGGTAYNQKFKG

SEQ ID NO: 303 - protein, 8 aa, Artificial Sequence
  ACI-7087-4119E10-Ab2 VH CDR3
FLLIDFDY

SEQ ID NO: 304 - protein, 112 aa, Artificial Sequence
  ACI-7087-4119E10-Ab2 VL
DVLMTQTPLSLPVSLGDQASISCRSSQSIVHSNGNTYLEWYLKKAGQSPKVLIYKVSNRFSGVPDRFSGSGSGTDFTLKISRVEAEDLGVYYCFQGSHVPYTFGGGTELEIK

SEQ ID NO: 305-306 - skipped (2 entries)

SEQ ID NO: 307 - protein, 9 aa, Artificial Sequence
  ACI-7087-4119E10-Ab2 VL CDR3
FQGSHVPYT

SEQ ID NO: 308 - DNA, 351 bp, Artificial Sequence
  ACI-7087-4119E10-Ab2 VH
caggttcaactgcagcagtctggggctgagctggtgaggcctggggcttcagtgacgctgtcctgcaaggcttcgggctacacattttctgactatgaaatgaactgggtgaagcagacacctgtgcatggcctggaatggattggagctattgatcctgaaactggtggtactgcctacaatcagaagttcaagggcaaggccatactgacttcagacaaatcctccagcacagcctacatggagctccgcagcctgacatctgaggactctgccgtctattactgtacaagattcctgttaatcgactttgactattggggccaaggcaccactctcacagtctcctca

SEQ ID NO: 309 - DNA, 336 bp, Artificial Sequence
  ACI-7087-4119E10-Ab2 VL
gatgtcttgatgacccaaactccactctccctgcctgtcagtcttggagatcaagcctccatctcttgcagatctagtcagagcattgtacatagtaatggaaacacctatttagaatggtacctgaagaaagcaggccagtctccaaaggtcctgatctacaaagtttccaaccgattttctggggtcccagacaggttcagtggcagtggatcagggacagatttcacactcaaaatcagcagggtggaggctgaggatctgggagtttattactgctttcaaggttcacatgttccgtacacattcggaggggggaccgagctggaaataaaa

SEQ ID NO: 310 - protein, 113 aa, Artificial Sequence
  ACI-7087-4125E6-Ab1 VH
QVQLQQPGTELVKPGASVRLSCKASGYAFTSYWMHWVKQRPGQGLEWIGNINPSNGGTNYNEKFKNKATLTVDKSSSTAYMQLSGLTSEDSAVYYCATGLHYWGQGTTLTVSS

SEQ ID NO: 311 - protein, 5 aa, Artificial Sequence
  ACI-7087-4125E6-Ab1 VH CDR1
SYWMH

SEQ ID NO: 312 - protein, 17 aa, Artificial Sequence
  ACI-7087-4125E6-Ab1 VH CDR2
NINPSNGGTNYNEKFKN

SEQ ID NO: 313 - protein, 4 aa, Artificial Sequence
  ACI-7087-4125E6-Ab1 VH CDR3
GLHY

SEQ ID NO: 314 - protein, 107 aa, Artificial Sequence
  ACI-7087-4125E6-Ab1 VL
DIQMTQSPSSLSASLGERVTLTCRASQDIGNYLNWLQQEPDGTIKRLIYATSSLDSGVPKRFSGSRSGSDYSLTISSLESEDFVDYYCLQFASSPLTFGPGTKLELK

SEQ ID NO: 315 - protein, 11 aa, Artificial Sequence
  ACI-7087-4125E6-Ab1 VL CDR1
RASQDIGNYLN

SEQ ID NO: 316-317 - skipped (2 entries)

SEQ ID NO: 318 - DNA, 339 bp, Artificial Sequence
  ACI-7087-4125E6-Ab1 VH
caggtccaactgcagcagcctgggactgaactggtgaagcctggggcttcagtgaggctgtcctgcaaggcttctggctacgccttcaccagctactggatgcactgggtgaagcagaggcctggacaaggccttgagtggattggaaatattaatcctagcaatggtggtactaactacaatgagaagttcaagaacaaggccacactgactgtagacaaatcctccagcacagcctatatgcagctcagcggcctgacatctgaggactctgcggtctattattgtgcaacgggccttcactactggggccaaggcaccactctcacagtctcctca

SEQ ID NO: 319 - DNA, 321 bp, Artificial Sequence
  ACI-7087-4125E6-Ab1 VL
gacatccagatgacccagtctccatcctccttatctgcctctctgggagaaagagtcactctcacttgtcgggcaagtcaggacattggtaattacttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatttgctagttctccgctcacgttcggtcctgggaccaaactggaactgaaa

SEQ ID NO: 320 - protein, 117 aa, Artificial Sequence
  ACI-7088-4301D5-Ab2 VH
QVQLQQSGPELVRPGASVKISCKASGYRFTSYYIHWVKQRPGQGLEWIGWIYPGSDNTKHNDKFKGKATLTADTSSSTAYMQLSSLTSEDSAVYFCARDYDVGFGYWGQGTLVTVSS

SEQ ID NO: 321 - protein, 5 aa, Artificial Sequence
  ACI-7088-4301D5-Ab2 VH CDR1
SYYIH

SEQ ID NO: 322 - protein, 17 aa, Artificial Sequence
  ACI-7088-4301D5-Ab2 VH CDR2
WIYPGSDNTKHNDKFKG

SEQ ID NO: 323 - protein, 8 aa, Artificial Sequence
  ACI-7088-4301D5-Ab2 VH CDR3
DYDVGFGY

SEQ ID NO: 324 - protein, 111 aa, Artificial Sequence
  ACI-7088-4301D5-Ab2 VL
DIVLTQSPASLAVSLGQRATISCRASESVDNYGISFMNWFQQKPGQPPKLLIYAASNQGSGVPARFSGIGSGTDFSLNIHPMEEDDTAMYFCQQSQEVPLTFGAGTKLELK

SEQ ID NO: 325 - protein, 15 aa, Artificial Sequence
  ACI-7088-4301D5-Ab2 VL CDR1
RASESVDNYGISFMN

SEQ ID NO: 326 - protein, 7 aa, Artificial Sequence
  ACI-7088-4301D5-Ab2 VL CDR2
AASNQGS

SEQ ID NO: 327 - protein, 9 aa, Artificial Sequence
  ACI-7088-4301D5-Ab2 VL CDR3
QQSQEVPLT

SEQ ID NO: 328 - DNA, 351 bp, Artificial Sequence
  ACI-7088-4301D5-Ab2 VH
caggtccagctgcagcagtctggacctgagctggtgaggcctggggcttcagtgaagatatcctgcaaggcttctggctacaggttcacaagctactatatacactgggtgaagcagaggcctggacagggacttgagtggattggatggatttatcctggaagtgataatactaagcacaatgacaagttcaagggcaaggccacactgacggcagacacatcctccagcactgcctacatgcagctcagcagcctaacatctgaggactctgcggtctatttctgtgcaagagactacgacgtggggtttggttactggggccaagggactctggtcactgtctctgca

SEQ ID NO: 329 - DNA, 333 bp, Artificial Sequence
  ACI-7088-4301D5-Ab2 VL
gacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcattgggtctgggacagacttcagcctcaacatccatcctatggaggaggatgatactgcaatgtatttctgtcagcaaagtcaggaggttccgctcacgttcggtgctgggaccaagctggagctgaaa

SEQ ID NO: 330 - protein, 113 aa, Artificial Sequence
  ACI-7088-4301E12-Ab2 VH
DVQLQESGPGLVKPSQSLSLTCSVTGYSITSGYYWNWIRQFPGNKLEWMGYISDDGSKNYNPSLKNRISITRDTSKNQLFMKLNSVTTEDTATYYCARGDSRLGQGTLVTVSA

SEQ ID NO: 331 - skipped

SEQ ID NO: 332 - protein, 16 aa, Artificial Sequence
  ACI-7088-4301E12-Ab2 VH CDR2
YISDDGSKNYNPSLKN

SEQ ID NO: 333 - protein, 4 aa, Artificial Sequence
  ACI-7088-4301E12-Ab2 VH CDR3
GDSR

SEQ ID NO: 334 - protein, 112 aa, Artificial Sequence
  ACI-7088-4301E12-Ab2 VL
DVVLTQTPLTLSVTIGQPASISCRSSQNLLDSDGETYLNWLLQRPGQSPKRLIYLVSELDSGVPDRFTGSGSGTDFTLKISRVEAEDLGVYYCWQGTHFPQTFGGGTKLEII

SEQ ID NO: 335 - protein, 16 aa, Artificial Sequence
  ACI-7088-4301E12-Ab2 VL CDR1
RSSQNLLDSDGETYLN

SEQ ID NO: 336 - protein, 7 aa, Artificial Sequence
  ACI-7088-4301E12-Ab2 VL CDR2
LVSELDS

SEQ ID NO: 337 - skipped

SEQ ID NO: 338 - DNA, 339 bp, Artificial Sequence
  ACI-7088-4301E12-Ab2 VH
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagcgacgatggtagtaaaaattacaacccatctctcaaaaatcgaatctccatcactcgtgacacatctaagaaccagcttttcatgaagttgaattctgtgactactgaggacacagccacatattactgtgcaagaggcgattcccgcctgggccaagggactctggtcactgtctctgca

SEQ ID NO: 339 - DNA, 336 bp, Artificial Sequence
  ACI-7088-4301E12-Ab2 VL
gatgttgtgttgacccagactccactcactttgtcggttaccattggacaaccagcctccatctcttgcaggtcaagtcagaacctcttagatagtgatggagagacatatttgaattggttgttacagaggccaggccagtctccaaagcgcctaatctatctggtgtctgagctggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcatt

SEQ ID NO: 340 - protein, 117 aa, Artificial Sequence
  ACI-7088-4301H3-Ab2 VH
EVQLQQSGPELVKPGASVKISCKASGYTFADYFMNWVKQSHGKSLEWIGDINPNNGGTTYNQKFKGKATLTVDKSSNTAYMELRSLTSEDSAVYYCARGRNYAMDYWGQGTSVTVSS

SEQ ID NO: 341 - protein, 5 aa, Artificial Sequence
  ACI-7088-4301H3-Ab2 VH CDR1
DYFMN

SEQ ID NO: 342 - protein, 17 aa, Artificial Sequence
  ACI-7088-4301H3-Ab2 VH CDR2
DINPNNGGTTYNQKFKG

SEQ ID NO: 343 - protein, 8 aa, Artificial Sequence
  ACI-7088-4301H3-Ab2 VH CDR3
GRNYAMDY

SEQ ID NO: 344 - protein, 112 aa, Artificial Sequence
  ACI-7088-4301H3-Ab2 VL
DIVMSQSPSSLAVSAGEKVTMSCKSSQSLLNSRTRKNYLAWYQQKPGQSPKLLIYSASTRESGVPDRFTGSGFGTDFTLTISSVQAEDLAVYYCKQSYDLWTFGGGTKLEIK

SEQ ID NO: 345 - protein, 17 aa, Artificial Sequence
  ACI-7088-4301H3-Ab2 VL CDR1
KSSQSLLNSRTRKNYLA

SEQ ID NO: 346 - protein, 7 aa, Artificial Sequence
  ACI-7088-4301H3-Ab2 VL CDR2
SASTRES

SEQ ID NO: 347 - protein, 8 aa, Artificial Sequence
  ACI-7088-4301H3-Ab2 VL CDR3
KQSYDLWT

SEQ ID NO: 348 - DNA, 351 bp, Artificial Sequence
  ACI-7088-4301H3-Ab2 VH
gaggtccagctgcaacaatctggacctgaactggtgaagcctggggcttcagtgaagatatcttgtaaggcttctggatacacgttcgctgactacttcatgaactgggtgaagcagagccatggaaagagccttgagtggattggagatattaatcctaacaatggtggtactacctacaaccagaagttcaagggcaaggccacattgactgtagacaagtcctccaacacagcctacatggagctccgcagcctgacatctgaggactctgcagtctactactgtgcaagaggtagaaactacgctatggactactggggtcaaggaacctcagtcaccgtctcctca

SEQ ID NO: 349 - DNA, 336 bp, Artificial Sequence
  ACI-7088-4301H3-Ab2 VL
gacattgtgatgtcacagtctccatcctccctggctgtgtcagcaggagagaaggtcactatgagctgcaaatccagtcagagtctcctcaacagtagaacccgaaagaattatttggcttggtaccagcagaaaccagggcagtctcctaaattgttgatctactcggcatccactagggaatctggggtccctgatcgcttcacaggcagtggatttgggacagatttcactctcaccatcagcagtgtgcaggctgaggacctggcagtttattactgcaagcaatcttatgatctgtggacgttcggtggaggcaccaagctggaaatcaaa

SEQ ID NO: 350 - protein, 119 aa, Artificial Sequence
  ACI-7088-4303A1-Ab1 VH
EVQLQQSGAELVRPGASVKLSCTASGFNIKDDYMHWVKQRPEQGLEWIGWIDPENGDSEYASKFQGKATMTADTSSNTAYLQLSSLTSEDTAVYYCKTWGTAQALFPYWGQGTLVTVSA

SEQ ID NO: 351 - protein, 5 aa, Artificial Sequence
  ACI-7088-4303A1-Ab1 VH CDR1
DDYMH

SEQ ID NO: 352 - protein, 17 aa, Artificial Sequence
  ACI-7088-4303A1-Ab1 VH CDR2
WIDPENGDSEYASKFQG

SEQ ID NO: 353 - protein, 10 aa, Artificial Sequence
  ACI-7088-4303A1-Ab1 VH CDR3
WGTAQALFPY

SEQ ID NO: 354 - protein, 107 aa, Artificial Sequence
  ACI-7088-4303A1-Ab1 VL
DIVMTQSQKFMSTSVGDRVSITCKASQNVGTSVGWYQQKAGQSPKLLIHSASNRYTGVPDRFTGSGSGTDFTLTINNMQSEDLADYFCQQYRSYPLTFGAGTKLELK

SEQ ID NO: 355 - protein, 11 aa, Artificial Sequence
  ACI-7088-4303A1-Ab1 VL CDR1
KASQNVGTSVG

SEQ ID NO: 356 - protein, 7 aa, Artificial Sequence
  ACI-7088-4303A1-Ab1 VL CDR2
SASNRYT

SEQ ID NO: 357 - protein, 9 aa, Artificial Sequence
  ACI-7088-4303A1-Ab1 VL CDR3
QQYRSYPLT

SEQ ID NO: 358 - DNA, 357 bp, Artificial Sequence
  ACI-7088-4303A1-Ab1 VH
gaggttcagctgcagcagtctggggctgaacttgtgaggccaggggcctcagtcaagttgtcctgcacagcttctggctttaacattaaagacgactatatgcactgggtgaaacagaggcctgaacagggcctggagtggattggatggattgatcctgagaatggtgattctgaatatgcctcgaagttccagggcaaggccactatgacagcagacacatcctccaacacagcctacctgcaactcagcagcctgacatctgaggacactgccgtctattattgtaaaacatgggggacagctcaggccctctttccttactggggccaagggactctggtcactgtctctgca

SEQ ID NO: 359 - DNA, 321 bp, Artificial Sequence
  ACI-7088-4303A1-Ab1 VL
gacattgtgatgacccagtctcaaaaattcatgtccacatcagtaggagacagggtcagcatcacctgcaaggccagtcagaatgtgggtacttctgtaggctggtatcaacaaaaagcaggacaatctcctaaactactgattcactcggcatctaatcggtacactggagtccctgatcgcttcacaggcagtggatctgggacagatttcactctcaccatcaacaatatgcagtctgaagacctggcagattatttctgccagcaatatagaagttatccgctcacgttcggtgctgggaccaagctggagctgaaa

SEQ ID NO: 360 - protein, 117 aa, Artificial Sequence
  ACI-7088-4303A3-Ab1 VH
QVQLQQSGAELVRPGASVTLSCKASGYTFTDYEMHWVKQTPVHGLEWIGVIDPETGGAVQNQKFKGKAILTADNSSSTAYMDLRSLTSEDSAVYNCAMGAALRLAYWGQGTLVTVSS

SEQ ID NO: 361 - protein, 5 aa, Artificial Sequence
  ACI-7088-4303A3-Ab1 VH CDR1
DYEMH

SEQ ID NO: 362 - protein, 17 aa, Artificial Sequence
  ACI-7088-4303A3-Ab1 VH CDR2
VIDPETGGAVQNQKFKG

SEQ ID NO: 363 - protein, 8 aa, Artificial Sequence
  ACI-7088-4303A3-Ab1 VH CDR3
GAALRLAY

SEQ ID NO: 364 - protein, 112 aa, Artificial Sequence
  ACI-7088-4303A3-Ab1 VL
DVLMTQTPLSLPVSLGDQASISCRSSQSIVHSNGNSYLEWYLQKPGQSPKLLIYKVSNRFSGVPDRFSGSGSGTDFTLKINRVEAEDLGVYYCFQGSHVPFTFGSGTKLEIK

SEQ ID NO: 365 - protein, 16 aa, Artificial Sequence
  ACI-7088-4303A3-Ab1 VL CDR1
RSSQSIVHSNGNSYLE

SEQ ID NO: 366 - skipped

SEQ ID NO: 367 - protein, 9 aa, Artificial Sequence
  ACI-7088-4303A3-Ab1 VL CDR3
FQGSHVPFT

SEQ ID NO: 368 - DNA, 351 bp, Artificial Sequence
  ACI-7088-4303A3-Ab1 VH
caggttcaactgcagcagtctggggctgagctggtgaggcctggggcttcagtgacgctgtcctgcaaggcttcgggctacacatttactgactatgaaatgcactgggtgaaacagacacctgtgcatggcctggagtggattggagttattgatcctgaaactggtggtgctgtccagaatcagaagttcaagggcaaggccatactgactgcagacaattcctccagcacagcctacatggacctccgcagcctgacatctgaggactctgccgtctataactgtgcaatgggtgcggcattacggcttgcttactggggccaagggactctggtcactgtctctgca

SEQ ID NO: 369 - DNA, 336 bp, Artificial Sequence
  ACI-7088-4303A3-Ab1 VL
gatgttttgatgacccaaactccactctccctgcctgtcagtcttggagatcaagcctccatctcttgcagatctagtcagagcattgtacatagtaatggaaactcctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccattcacgttcggctcggggacaaagttggaaataaaa

SEQ ID NO: 370 - protein, 123 aa, Artificial Sequence
  ACI-7088-4303B6-Ab2 VH
EVQLQQSGPELVKPGASVKISCKASGYTFTDYYMNWVKQSHGKSLEWIGDINPNNGGTTYNQKFKDKATLTVDRSSSTAYMELRSLTSGDSAVYYCARSGYSGSRLYYAMDYWSQGSSVTVSS

SEQ ID NO: 371 - protein, 5 aa, Artificial Sequence
  ACI-7088-4303B6-Ab2 VH CDR1
DYYMN

SEQ ID NO: 372 - protein, 17 aa, Artificial Sequence
  ACI-7088-4303B6-Ab2 VH CDR2
DINPNNGGTTYNQKFKD

SEQ ID NO: 373 - protein, 14 aa, Artificial Sequence
  ACI-7088-4303B6-Ab2 VH CDR3
SGYSGSRLYYAMDY

SEQ ID NO: 374 - protein, 112 aa, Artificial Sequence
  ACI-7088-4303B6-Ab2 VL
DIVMSQSPSSLAVSAREKVTMSCKSSQSLLNSRTRKNYLAWYQQKPGQSPKLLIFWASTRESGVPDRFTGSGSGTDFTLTISSVQAEDLAVYYCKQSYDLWTFGGGTKLEIK

SEQ ID NO: 375 - skipped

SEQ ID NO: 376 - protein, 7 aa, Artificial Sequence
  ACI-7088-4303B6-Ab2 VL CDR2
WASTRES

SEQ ID NO: 377 - skipped

SEQ ID NO: 378 - DNA, 369 bp, Artificial Sequence
  ACI-7088-4303B6-Ab2 VH
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactacctacaaccagaagttcaaggacaaggccacattgactgtggacaggtcctccagcacagcctacatggaactccgcagcctgacatctggggactctgcagtctattactgtgcaagatcggggtactccggtagtcgcctctactatgctatggactactggagtcaaggatcctcagtcaccgtctcctca

SEQ ID NO: 379 - DNA, 336 bp, Artificial Sequence
  ACI-7088-4303B6-Ab2 VL
gacattgtgatgtcacagtctccatcctccctggctgtgtcagcacgagagaaggtcactatgagctgcaaatccagtcagagtctgctcaacagtagaacccgaaagaactacttggcttggtaccagcagaaaccagggcagtctcctaaactgctgatcttctgggcttccactagggaatctggggtccctgatcgcttcactggcagtggatctgggacagatttcactctcaccatcagcagtgtgcaggctgaagacctggcagtttattactgcaaacaatcttatgatctgtggacgttcggtggcggcaccaagctggaaatcaaa

SEQ ID NO: 380 - protein, 119 aa, Artificial Sequence
  ACI-7088-4303H6-Ab1 VH
EVQLQQSGAELVRPGASVKLSCTASGFNIQDDYMHWVKQRPEQGLEWIGWIDPENGDTEYASKFQGKATLTADTSSNTAYLQLSRLTSEDTAVYYCTTAGSGVQLFDYWGQGTTLTVSA

SEQ ID NO: 381 - skipped

SEQ ID NO: 382 - protein, 17 aa, Artificial Sequence
  ACI-7088-4303H6-Ab1 VH CDR2
WIDPENGDTEYASKFQG

SEQ ID NO: 383 - protein, 10 aa, Artificial Sequence
  ACI-7088-4303H6-Ab1 VH CDR3
AGSGVQLFDY

SEQ ID NO: 384 - protein, 107 aa, Artificial Sequence
  ACI-7088-4303H6-Ab1 VL
DILMTQSQKFMSTSVGDRVSVTCKASQNVGTNVAWYQQKPGQSPKPLISSASSRYSGVPDRFTGSGSGTDFTLTISNVQSEDLADYFCQQYNRYPLTFGAGTKLELK

SEQ ID NO: 385 - protein, 11 aa, Artificial Sequence
  ACI-7088-4303H6-Ab1 VL CDR1
KASQNVGTNVA

SEQ ID NO: 386 - protein, 7 aa, Artificial Sequence
  ACI-7088-4303H6-Ab1 VL CDR2
SASSRYS

SEQ ID NO: 387 - protein, 9 aa, Artificial Sequence
  ACI-7088-4303H6-Ab1 VL CDR3
QQYNRYPLT

SEQ ID NO: 388 - DNA, 357 bp, Artificial Sequence
  ACI-7088-4303H6-Ab1 VH
gaggttcagctgcagcagtctggggctgagcttgtgaggccaggggcctcagtcaagttgtcctgcacagcttctggctttaacattcaagacgactatatgcactgggtgaagcagaggcctgaacagggcctggagtggattggttggattgatcctgagaatggtgatactgaatatgcctcgaaattccagggcaaggccactttaacagcagacacatcctccaacacagcctacctgcagctcagcagactgacatctgaggacactgccgtctattactgtactacagcgggctcaggcgtccaactctttgactactggggccaaggcaccactctcacagtctcctca

SEQ ID NO: 389 - DNA, 321 bp, Artificial Sequence
  ACI-7088-4303H6-Ab1 VL
gacattttgatgacccagtctcaaaaattcatgtccacatcagtaggagacagggtcagcgtcacctgcaaggccagtcagaatgtgggtactaatgtagcctggtatcaacagaaaccagggcaatctcctaaaccactgatttcctcggcatcctcccggtacagtggcgtccctgatcgcttcacaggcagtggatctgggacagatttcactctcaccatcagcaatgtgcagtctgaagacttggcagactatttctgtcagcaatataaccgctatcctctcacgttcggtgctgggaccaagctggagctgaaa

SEQ ID NO: 390 - protein, 119 aa, Artificial Sequence
  ACI-7088-4305H7-Ab1 VH
EVQLQQSGAELVRPGASVKLSCTASGFNIKDDYMHWVKQRPEQGLEWIGWIDPENGDTEYASKFQGKATMIADTSSNTAYLQLSSLTSEDTAVYYCKTWGTTQALFPYWGQGTLVTVSS

SEQ ID NO: 391-392 - skipped (2 entries)

SEQ ID NO: 393 - protein, 10 aa, Artificial Sequence
  ACI-7088-4305H7-Ab1 VH CDR3
WGTTQALFPY

SEQ ID NO: 394 - protein, 107 aa, Artificial Sequence
  ACI-7088-4305H7-Ab1 VL
DIVMTQSQKFMSTSVGDRVSITCKASQNVGTAVGWYQQKAGQSPKLLIHSASNRYTGVPDRFTGSGSGTDFTLTINNMQSEDLADYFCQQYRSYPLTFGAGTKLELK

SEQ ID NO: 395 - protein, 11 aa, Artificial Sequence
  ACI-7088-4305H7-Ab1 VL CDR1
KASQNVGTAVG

SEQ ID NO: 396-397 - skipped (2 entries)

SEQ ID NO: 398 - DNA, 357 bp, Artificial Sequence
  ACI-7088-4305H7-Ab1 VH
gaggttcagctgcagcagtctggggctgaacttgtgaggccaggggcctcagtcaagttgtcctgcacagcttctggctttaacattaaagacgactatatgcactgggtgaagcagaggcctgaacagggcctggagtggattggatggattgatcctgagaatggtgatactgaatatgcctcgaagttccagggcaaggccactatgatagcagacacatcctccaacacagcctacctgcaactcagcagcctgacatctgaggacactgccgtctattattgtaaaacatgggggacaactcaggccctctttccttactggggccaagggactctggtcactgtctctgca

SEQ ID NO: 399 - DNA, 321 bp, Artificial Sequence
  ACI-7088-4305H7-Ab1 VL
gacattgtgatgacccagtctcaaaaattcatgtccacatcagtaggagacagggtcagcatcacctgcaaggccagtcagaatgtgggtactgctgtaggctggtatcaacaaaaagcaggacaatctcctaaactactgattcactcggcatccaatcggtacactggagtccctgatcgcttcacaggcagtggatctgggacagatttcactctcaccatcaacaatatgcagtctgaagacctggcagattatttctgccagcaatatagaagttatccgctcacgttcggtgctgggaccaagctggagctgaaa

SEQ ID NO: 400 - protein, 119 aa, Artificial Sequence
  ACI-7088-4317A4-Ab1 VH
EVQLQQSGAELVRPGASVKLSCTASGFNIKDDYMHWVKQRPEQGLEWIGWIDPENGDTEYASKFQGKATMTADTSSNTAYLQLSSLTSEDTAVYYCKTWGTTQALFPYWGQGTLVTVSA

SEQ ID NO: 401-403 - skipped (3 entries)

SEQ ID NO: 404 - protein, 107 aa, Artificial Sequence
  ACI-7088-4317A4-Ab1 VL
DIVMTQSQKFMYTSVGDRVSITCKASQNVGNAVGWYQQKAGQSPKLLIHSASNRYTGVPDRFTGTGSGTDFTLTINNMQSEDLADYFCQQYRSYPLTFGAGTKLELK

SEQ ID NO: 405 - protein, 11 aa, Artificial Sequence
  ACI-7088-4317A4-Ab1 VL CDR1
KASQNVGNAVG

SEQ ID NO: 406-407 - skipped (2 entries)

SEQ ID NO: 408 - DNA, 357 bp, Artificial Sequence
  ACI-7088-4317A4-Ab1 VH
gaggttcagctgcagcagtctggggctgaacttgtgaggccaggggcctcagtcaagttgtcctgcacagcttctggctttaacattaaagacgactatatgcactgggtgaagcagaggcctgaacagggcctggagtggattggatggattgatcctgagaatggtgatactgaatatgcctcgaagttccagggcaaggccactatgacagcagacacatcctccaacacagcctacctgcaactcagcagcctgacatctgaggacactgccgtctattattgtaaaacatgggggacaactcaggccctctttccttactggggccaagggactctggtcactgtctctgca

SEQ ID NO: 409 - DNA, 321 bp, Artificial Sequence
  ACI-7088-4317A4-Ab1 VL
gacattgtgatgacccagtctcaaaagttcatgtacacatcagtgggagacagggtcagcatcacctgcaaggccagtcagaatgtgggtaatgctgtaggctggtatcaacaaaaagcaggacaatctcctaaactactgattcactcggcatccaatcggtacactggagtccctgatcgcttcacaggcactggatctgggacagatttcactctcaccatcaacaatatgcagtctgaagacctggcagattatttctgccagcaatatagaagttatccgctcacgttcggtgctgggaccaagctggagctgaaa

SEQ ID NO: 410 - protein, 113 aa, Artificial Sequence
  ACI-7089-4409F1-Ab1 VH
DVQLQESGPGLVKPSQSLSLTCSVTGYSITRGYYWNWIRQFPGNKLEWMGYISYDGSNNYNPSLRNRISITRDTSKNQFFLKLKSVTTEDTATYFCARGDSNWGQGTTLTVSA

SEQ ID NO: 411 - protein, 6 aa, Artificial Sequence
  ACI-7089-4409F1-Ab1 VH CDR1
RGYYWN

SEQ ID NO: 412 - protein, 16 aa, Artificial Sequence
  ACI-7089-4409F1-Ab1 VH CDR2
YISYDGSNNYNPSLRN

SEQ ID NO: 413 - protein, 4 aa, Artificial Sequence
  ACI-7089-4409F1-Ab1 VH CDR3
GDSN

SEQ ID NO: 414 - protein, 112 aa, Artificial Sequence
  ACI-7089-4409F1-Ab1 VL
DVVMTQTPLTLSVTIGQPASISCKSSQSLLDSDGETYLNWLLQRPGQSPKRLIYLVSKLDSGVPDRFTGSGSGTDFTLKISRVEAEDLGVYYCWQGTHFPQTFGGGTKLEIK

SEQ ID NO: 415-417 - skipped (3 entries)

SEQ ID NO: 418 - DNA, 339 bp, Artificial Sequence
  ACI-7089-4409F1-Ab1 VH
gatgtacagcttcaggagtcaggacctggcctcgtgaaaccttctcagtctctgtctctcacctgctctgtcactggctactccatcaccaggggttattactggaactggatccggcagtttccaggaaacaaactggaatggatgggctacataagctacgatggtagcaataactacaacccatctctcagaaatcgaatctccatcactcgtgacacatctaagaaccagtttttcctgaagttgaaatctgtgactactgaggacacagccacatatttctgtgcaagaggggatagtaactggggccaaggcaccactctcacagtctcctca

SEQ ID NO: 419 - DNA, 336 bp, Artificial Sequence
  ACI-7089-4409F1-Ab1 VL
gatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctatctggtgtctaaactggactctggagtccctgacaggttcactggtagtggatcagggacagatttcacactgaaaatcagcagagtggaggctgaggatttgggagtttattattgctggcaaggtacacattttcctcagacgttcggtggaggcaccaagttggaaatcaaa

SEQ ID NO: 420 - protein, 112 aa, Artificial Sequence
  ACI-7089-4415G5-Ab1 VH
QVQLQQSGAELARPGASVKVSCKASGYTFTSSGISWLKHRTGQGLEWIGDIYPRSGNTYYNEKFKDKATLTADKSSSTAYMELRSLTSEDSAVYFCASGNYWGQGTTLTVSA

SEQ ID NO: 421 - protein, 5 aa, Artificial Sequence
  ACI-7089-4415G5-Ab1 VH CDR1
SSGIS

SEQ ID NO: 422 - protein, 17 aa, Artificial Sequence
  ACI-7089-4415G5-Ab1 VH CDR2
DIYPRSGNTYYNEKFKD

SEQ ID NO: 423 - skipped

SEQ ID NO: 424 - protein, 112 aa, Artificial Sequence
  ACI-7089-4415G5-Ab1 VL
DIVITQDDLSNPVTSGESVSISCRSSKSLLYKDGKTYLNWFLQRPGQSPQLLIYLMSTRASGVSDRFSGSGSGTDFTLEISRVKAEDVGVYYCQQLLEYPLTFGAGTKLELK

SEQ ID NO: 425 - protein, 16 aa, Artificial Sequence
  ACI-7089-4415G5-Ab1 VL CDR1
RSSKSLLYKDGKTYLN

SEQ ID NO: 426 - protein, 7 aa, Artificial Sequence
  ACI-7089-4415G5-Ab1 VL CDR2
LMSTRAS

SEQ ID NO: 427 - protein, 9 aa, Artificial Sequence
  ACI-7089-4415G5-Ab1 VL CDR3
QQLLEYPLT

SEQ ID NO: 428 - DNA, 336 bp, Artificial Sequence
  ACI-7089-4415G5-Ab1 VH
caggttcagctgcagcagtctggagctgagctggcgaggcctggggcttcagtgaaggtgtcctgcaaggcttctggctacaccttcacaagctctggtataagctggttgaagcacagaactggacagggccttgagtggattggagacatttatcctagaagtggtaatacttactacaatgagaaattcaaggacaaggccacactgactgcagacaaatcctccagcacggcgtacatggagctccgcagcctgacatctgaggactctgcggtctatttctgtgcaagtggtaactactggggccaaggcaccactctcacagtctcctca

SEQ ID NO: 429 - DNA, 336 bp, Artificial Sequence
  ACI-7089-4415G5-Ab1 VL
gatattgtgataacccaggatgacctctccaatcctgtcacttctggagaatcagtttccatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaacttttagagtatccgctcacgttcggtgctgggaccaagctggagctgaaa

SEQ ID NO: 430 - protein, 114 aa, Artificial Sequence
  ACI-7089-4417G6-Ab1 VH
QVQLQQSGAELVRPGASVTLSCKASGYTFTGYEMHKQTPVHGLEWIGAIDPETGGTAYIQKFKGKATLTADKSSSTAYMELRSLTSEDSAVYYCTRGWDYFDYWGQGTTLTVSA

SEQ ID NO: 431 - protein, 5 aa, Artificial Sequence
  ACI-7089-4417G6-Ab1 VH CDR1
GYEMH

SEQ ID NO: 432 - protein, 17 aa, Artificial Sequence
  ACI-7089-4417G6-Ab1 VH CDR2
AIDPETGGTAYIQKFKG

SEQ ID NO: 433 - protein, 7 aa, Artificial Sequence
  ACI-7089-4417G6-Ab1 VH CDR3
GWDYFDY

SEQ ID NO: 434 - protein, 112 aa, Artificial Sequence
  ACI-7089-4417G6-Ab1 VL
DVVMTQTPLSLPVSLGDQASISCRSSQSLLHSNGFTYLHWYLQKPGQSPKLLIYRVSNRFSGVPDRFSGSGSGTDFTLKISRVEAEDLGVYFCSQSTHVPYTFGGGTKLEIK

SEQ ID NO: 435 - protein, 16 aa, Artificial Sequence
  ACI-7089-4417G6-Ab1 VL CDR1
RSSQSLLHSNGFTYLH

SEQ ID NO: 436 - protein, 7 aa, Artificial Sequence
  ACI-7089-4417G6-Ab1 VL CDR2
RVSNRFS

SEQ ID NO: 437 - protein, 9 aa, Artificial Sequence
  ACI-7089-4417G6-Ab1 VL CDR3
SQSTHVPYT

SEQ ID NO: 438 - DNA, 348 bp, Artificial Sequence
  ACI-7089-4417G6-Ab1 VH
caggttcaactgcagcagtctggggctgagttggtgaggcctggggcttcagtgacgctgtcctgcaaggcttcgggctacacatttactggctatgaaatgcactgggtgaagcagacacctgtgcatggcctggaatggattggagctattgatcctgaaaccggtggaactgcctatattcagaagttcaagggcaaggccacactgactgcagacaaatcctccagcacagcctacatggagctccgcagcctgacatctgaggactctgccgtctattactgtacaagaggctgggactattttgactactggggccaaggcaccactctcacagtctcctca

SEQ ID NO: 439 - DNA, 336 bp, Artificial Sequence
  ACI-7089-4417G6-Ab1 VL
gatgttgtgatgacccaaactccactctccctgcctgtcagtcttggagatcaagcctccatctcttgcagatctagtcagagccttctacacagtaatggattcacctatttacattggtacctgcagaagccaggccagtctccaaagctcctgatctacagagtttccaatcgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaa

SEQ ID NO: 440 - protein, 113 aa, Artificial Sequence
  ACI-7089-4418C5-Ab1 VH
DGQLQESGPGLVKPSQSLSLTCSVTGYSITSGYYWNWIRQFPGNKLEWMGYINYDGSNNYNPSLKNRISITRDTSKNQFFLKFNFVTTEDTATYYCVRGDVYWGQGTTLTVSS

SEQ ID NO: 441 - skipped

SEQ ID NO: 442 - protein, 16 aa, Artificial Sequence
  ACI-7089-4418C5-Ab1 VH CDR2
YINYDGSNNYNPSLKN

SEQ ID NO: 443 - protein, 4 aa, Artificial Sequence
  ACI-7089-4418C5-Ab1 VH CDR3
GDVY

SEQ ID NO: 444-447 - skipped (4 entries)

SEQ ID NO: 448 - DNA, 339 bp, Artificial Sequence
  ACI-7089-4418C5-Ab1 VH
gatggacaacttcaggagtcaggacctggcctcgtgaaaccttctcagtctctgtctctcacctgctctgtcactggctactccatcaccagtggatattactggaactggatccggcagtttccaggaaacaaactggaatggatgggctacataaactacgatggtagcaataactacaacccatctctcaaaaatcgaatctccatcactcgtgacacatctaagaatcagtttttcctgaagttcaattttgtgactactgaggacacagccacatattactgtgtgaggggggacgtctactggggccaaggcaccactctcacagtctcctca

SEQ ID NO: 449 - DNA, 336 bp, Artificial Sequence
  ACI-7089-4418C5-Ab1 VL
gatgttgtgatgacccagactccactcactttgtcggttaccattggacaaccagcctccatctcttgcaagtcaagtcagagcctcttagatagtgatggagagacatatttgaattggttattacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 450 - protein, 112 aa, Artificial Sequence
  ACI-7089-4418F6-Ab1 VH
QVQLQQSGAELARPGASVKVSCKASGYTFTSSGISWLKHRTGQGLEWIGDIYPRSGNTYYNEKFKDKATLTADKSSSTAYMELRSLTSEDSAVYFCSSGNYWGQGTTLTVSS

SEQ ID NO: 451-457 - skipped (7 entries)

SEQ ID NO: 458 - DNA, 336 bp, Artificial Sequence
  ACI-7089-4418F6-Ab1 VH
caggttcagctgcagcagtctggagctgagctggcgaggcctggggcttcagtgaaggtgtcctgcaaggcttctggctacaccttcacaagttctggtataagctggttgaagcacagaactggacagggccttgagtggattggagacatttatcctagaagtggtaatacttactacaatgagaaattcaaggacaaggccacactgactgcagacaaatcctccagcacggcgtacatggagctccgcagcctgacatctgaggactctgcggtctatttctgttcaagtggtaactactggggccaaggcaccactctcacagtctcctca

SEQ ID NO: 459 - DNA, 336 bp, Artificial Sequence
  ACI-7089-4418F6-Ab1 VL
gatattgtgataacccaggatgacctctccaatcctgtcacttctggagaatcagtttccatctcctgtaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaacttttagagtatccgctcacgttcggtgctgggaccaagctggagctgaaa

SEQ ID NO: 460 - protein, 114 aa, Artificial Sequence
  ACI-8033-5A12-Ab1 VH
QVHLKQSGADLVRPGASVKLSCKASGYTFTDYYINWVKQRPGQGLEWIARIYPGSGNTYYNEKFKGRATLSAEKSSTTAYMQLSSLTSEDSAVYFCVVGYYGAWGQGTTLTVSS

SEQ ID NO: 461 - protein, 5 aa, Artificial Sequence
  ACI-8033-5A12-Ab1 VH CDR1
DYYIN

SEQ ID NO: 462 - protein, 17 aa, Artificial Sequence
  ACI-8033-5A12-Ab1 VH CDR2
RIYPGSGNTYYNEKFKG

SEQ ID NO: 463 - protein, 5 aa, Artificial Sequence
  ACI-8033-5A12-Ab1 VH CDR3
GYYGA

SEQ ID NO: 464 - protein, 112 aa, Artificial Sequence
  ACI-8033-5A12-Ab1 VL
DVVMTQTPLSLPVSLGDQASISCRSSQSLVHSNGKTHLHWYLQKPGQSPKLLIYKVSNRFSGVPDRFSGSGSGTDFTLKISRVEAEDLGVYFCSQSTHVPWTFGGGTKLEIK

SEQ ID NO: 465 - protein, 16 aa, Artificial Sequence
  ACI-8033-5A12-Ab1 VL CDR1
RSSQSLVHSNGKTHLH

SEQ ID NO: 466 - skipped

SEQ ID NO: 467 - protein, 9 aa, Artificial Sequence
  ACI-8033-5A12-Ab1 VL CDR3
SQSTHVPWT

SEQ ID NO: 468 - DNA, 342 bp, Artificial Sequence
  ACI-8033-5A12-Ab1 VH
caggtccacctgaagcagtctggggctgacctggtgaggcctggggcttcagtgaagctgtcctgcaaggcgtctggctacactttcactgactactatataaactgggtgaagcagaggcctggacagggacttgagtggattgcaaggatttatcctggaagtggtaatacttactacaatgagaagttcaagggcagggccacactgagtgcagaaaaatcctccaccactgcctacatgcagctcagcagcctgacatctgaggactctgctgtctatttctgtgtagtggggtactacggtgcctggggccaaggcaccactctcacagtctcctca

SEQ ID NO: 469 - DNA, 336 bp, Artificial Sequence
  ACI-8033-5A12-Ab1 VL
gatgttgtgatgacccaaactccactctccctgcctgtcagtcttggagatcaagcctccatctcttgtagatctagtcagagccttgtacacagtaatggaaaaacccatttacattggtacctgcagaagccaggccagtctccaaagctcctgatctataaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 470 - protein, 115 aa, Artificial Sequence
  ACI-8033-25A3-Ab1 VH
EVQLVESGGGLVQPKGSLKLSCAASGFSFNTYAMNWVRQAPGKGLEWVARIRSKSNNFATYYADSVKDRFTISRDESESMLYLQMNNLKTEDTAMYYCVRSFDYWGQGTTLTVSS

SEQ ID NO: 471 - skipped

SEQ ID NO: 472 - protein, 19 aa, Artificial Sequence
  ACI-8033-25A3-Ab1 VH CDR2
RIRSKSNNFATYYADSVKD

SEQ ID NO: 473 - protein, 4 aa, Artificial Sequence
  ACI-8033-25A3-Ab1 VH CDR3
SFDY

SEQ ID NO: 474 - protein, 107 aa, Artificial Sequence
  ACI-8033-25A3-Ab1 VL
DIKMTQSPSSMYASLGERVTITCKASQDINSYLSWFQQKPGKSPKTLIYRAKRLVDGVPSRFSGSGSGQDYSLTISSLEYEDMGIYYCLQYDEFPFTFGSGTKLEIK

SEQ ID NO: 475 - protein, 11 aa, Artificial Sequence
  ACI-8033-25A3-Ab1 VL CDR1
KASQDINSYLS

SEQ ID NO: 476 - protein, 7 aa, Artificial Sequence
  ACI-8033-25A3-Ab1 VL CDR2
RAKRLVD

SEQ ID NO: 477 - protein, 9 aa, Artificial Sequence
  ACI-8033-25A3-Ab1 VL CDR3
LQYDEFPFT

SEQ ID NO: 478 - DNA, 345 bp, Artificial Sequence
  ACI-8033-25A3-Ab1 VH
gaggtgcagcttgttgagtctggtggaggattggtgcagcctaaagggtcattgaaactctcatgtgcagcctctggattcagcttcaatacctacgccatgaactgggtccgccaggctccaggaaagggtttggaatgggttgctcgcataagaagtaaaagtaataattttgcaacatattatgccgattcagtgaaagacagattcaccatctccagagatgaatcagaaagcatgctctatctgcaaatgaacaacttgaaaactgaggacacagccatgtattactgtgtgaggtcctttgactactggggccaaggcaccactctcacagtctcctca

SEQ ID NO: 479 - DNA, 321 bp, Artificial Sequence
  ACI-8033-25A3-Ab1 VL
gacatcaagatgacccagtctccatcttccatgtatgcatctctaggagagagagtcactatcacttgcaaggcgagtcaggacattaatagctatttaagctggttccagcagaaaccagggaaatctcctaagaccctaatctatcgtgcaaaaagattggtagatggggtcccatcaaggttcagtggcagtggatctgggcaagattattctctcaccatcagcagcctggagtatgaagatatgggaatttattattgtctacagtatgatgagtttccattcacgttcggctcggggacaaagttggaaataaaa

SEQ ID NO: 480 - protein, 119 aa, Artificial Sequence
  ACI-8033-1G10-Ab1 VH
DVQLQESGPGLVKPSQTVFLTCTVTGISITTGNYRWSWIRQFPGNKLEWIGYIYYSGTITYNPSLTSRTTITRDTPKNQFFLEMNSLTAEDTATYYCARIYYGNAMDYWGQGTSVTVSS

SEQ ID NO: 481 - protein, 7 aa, Artificial Sequence
  ACI-8033-1G10-Ab1 VH CDR1
TGNYRWS

SEQ ID NO: 482 - protein, 16 aa, Artificial Sequence
  ACI-8033-1G10-Ab1 VH CDR2
YIYYSGTITYNPSLTS

SEQ ID NO: 483 - protein, 9 aa, Artificial Sequence
  ACI-8033-1G10-Ab1 VH CDR3
IYYGNAMDY

SEQ ID NO: 484 - protein, 112 aa, Artificial Sequence
  ACI-8033-1G10-Ab1 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPHTFGGGTKLEIK

SEQ ID NO: 485-486 - skipped (2 entries)

SEQ ID NO: 487 - protein, 9 aa, Artificial Sequence
  ACI-8033-1G10-Ab1 VL CDR3
SQSTHVPHT

SEQ ID NO: 488 - DNA, 357 bp, Artificial Sequence
  ACI-8033-1G10-Ab1 VH
gatgtgcagcttcaggagtcaggacctggcctggtgaaaccttctcagacagtgttcctcacctgcactgtcactggcatttccatcaccactggaaattacaggtggagctggatccggcagtttccaggaaacaaactggagtggatagggtacatatactacagtggtaccattacctacaatccatctctcacaagtcgaaccaccatcactagagacactcccaagaaccagttcttcctggaaatgaactctttgactgctgaggacacagccacatactactgtgcacggatttactacggtaatgctatggactactggggtcaaggaacctcagtcaccgtctcctca

SEQ ID NO: 489 - DNA, 336 bp, Artificial Sequence
  ACI-8033-1G10-Ab1 VL
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctcacacgttcggaggggggaccaagctggaaataaaa

SEQ ID NO: 490 - protein, 115 aa, Artificial Sequence
  ACI-8033-19A2-Ab1 VH
EVQLVESGGGLVQPKGSLKLSCAASGFSFNTYAMNWVRQAPGKGLEWVARIRSKSNNYATYYVDSVKDRFTISRDDSESMLYLQMNNLKTEDTALYYCVSESAYWGQGTLVTVSA

SEQ ID NO: 491 - skipped

SEQ ID NO: 492 - protein, 19 aa, Artificial Sequence
  ACI-8033-19A2-Ab1 VH CDR2
RIRSKSNNYATYYVDSVKD

SEQ ID NO: 493 - protein, 4 aa, Artificial Sequence
  ACI-8033-19A2-Ab1 VH CDR3
ESAY

SEQ ID NO: 494 - protein, 112 aa, Artificial Sequence
  ACI-8033-19A2-Ab1 VL
DVVMTQTPLSLPVSLGDQASISCRSSQSLVHSNGNTYLYWYLQKPGQSPKLLIYKVSNRLSGVPDRFSGSGSGTDFTLKISRVEAEDLGVYFCSQSTHVPFTFGSGTKLEIK

SEQ ID NO: 495 - protein, 16 aa, Artificial Sequence
  ACI-8033-19A2-Ab1 VL CDR1
RSSQSLVHSNGNTYLY

SEQ ID NO: 496 - protein, 7 aa, Artificial Sequence
  ACI-8033-19A2-Ab1 VL CDR2
KVSNRLS

SEQ ID NO: 497 - protein, 9 aa, Artificial Sequence
  ACI-8033-19A2-Ab1 VL CDR3
SQSTHVPFT

SEQ ID NO: 498 - DNA, 345 bp, Artificial Sequence
  ACI-8033-19A2-Ab1 VH
gaggtgcaacttgttgagtctggtggaggattggtgcagcctaaagggtcattgaaactctcatgtgcagcctctggattcagcttcaatacctacgccatgaactgggtccgccaggctccaggaaagggtttggaatgggttgctcgcataagaagtaaaagtaataattatgcaacatattatgtcgattcagtgaaagacagattcaccatctccagagatgattcagaaagcatgctctatctgcaaatgaacaacttgaaaactgaggacacagccctgtattactgtgtgagcgaatccgcttactggggccaagggactctggtcactgtctctgca

SEQ ID NO: 499 - DNA, 336 bp, Artificial Sequence
  ACI-8033-19A2-Ab1 VL
gatgttgtgatgacccaaactccactctccctgcctgtcagtcttggagatcaagcctccatctcttgcagatctagtcagagccttgtacacagtaatggaaacacctatttatattggtacctgcagaagccaggccagtctccaaagctcctgatctacaaagtttccaaccgactttctggggtcccagacaggttcagtggcagtggatcagggacagatttcacactcaagatcagcagagtggaggctgaggatctgggagtttatttctgctctcaaagtacacatgttccattcacgttcggctcggggacaaagttggaaataaaa

SEQ ID NO: 500 - protein, 113 aa, Artificial Sequence
  ACI-8033-8C10-Ab1 VH
DVQLQESGPGLVKPSQSLSLTCSVTGQSITSGYYWNWIRQFPGNKLEWMGYISNDGSSKTNPSLTNRISVTRDTSKNQVFLKLKSVTTEDTATYYCVRGDQHWGQGTALTVSS

SEQ ID NO: 501 - skipped

SEQ ID NO: 502 - protein, 16 aa, Artificial Sequence
  ACI-8033-8C10-Ab1 VH CDR2
YISNDGSSKTNPSLTN

SEQ ID NO: 503 - protein, 4 aa, Artificial Sequence
  ACI-8033-8C10-Ab1 VH CDR3
GDQH

SEQ ID NO: 504 - protein, 112 aa, Artificial Sequence
  ACI-8033-8C10-Ab1 VL
DVVLTQTPLTLSVTIGQPASISCKSSQSLLDSDGETYLNWLLQRPGQSPKRLIYLVSELDSGVSDRFTGSGSGTDFTLKISRLEAEDLGVYYCWQGTHFPQTFGGGTKLEIK

SEQ ID NO: 505-507 - skipped (3 entries)

SEQ ID NO: 508 - DNA, 339 bp, Artificial Sequence
  ACI-8033-8C10-Ab1 VH
gatgtacagcttcaggagtcaggacctggcctcgtgaaaccttctcagtctctgtctctcacctgctctgtcactggccaatccatcaccagtggttattactggaactggatccggcaatttccaggaaacaaactggaatggatgggctacataagcaacgatggtagcagtaaaaccaacccatctctcacaaatcgaatctccgtcactcgtgacacatctaagaaccaggttttcctgaagttgaaatctgtgactactgaggacacagccacatattactgtgtaagaggggaccagcactggggccaaggcaccgctctcacagtctcctca

SEQ ID NO: 509 - DNA, 336 bp, Artificial Sequence
  ACI-8033-8C10-Ab1 VL
gatgttgtgttgacccagactccactcactttgtcagttaccattgggcaaccagcctccatctcttgcaagtcaagtcagagcctcttagatagtgatggagagacatatttgaattggttgttacagaggccaggccagtctccaaagcgcctaatctatctggtgtctgaactggactctggagtctctgacaggttcactggcagtggttcagggacagatttcacactgaaaatcagcagactggaggctgaggatttgggagtttattattgctggcaaggtacacattttcctcagacgttcggtggaggcaccaagctggaaatcaaa

SEQ ID NO: 510 - protein, 120 aa, Artificial Sequence
  ACI-8033-7A2-Ab1 VH
QVQLQQPGTELVKPGASVNLPCKASGYTFTSYWMHWVKQRPGQGLDWIGNVNPNNSDSNYNEKFKRKATLTVDKSSSTAYMHLSSLTSEDSAVYYCARSPYYGGRYLDYWGQGTTLTVSS

SEQ ID NO: 511 - skipped

SEQ ID NO: 512 - protein, 17 aa, Artificial Sequence
  ACI-8033-7A2-Ab1 VH CDR2
NVNPNNSDSNYNEKFKR

SEQ ID NO: 513 - protein, 11 aa, Artificial Sequence
  ACI-8033-7A2-Ab1 VH CDR3
SPYYGGRYLDY

SEQ ID NO: 514 - protein, 113 aa, Artificial Sequence
  ACI-8033-7A2-Ab1 VL
DIVMSQSPSSLAVSVGEKVTMTCKSSQSLLYRSNQKNYLAWYQQKPGQSPKLLIYWAFTRESGVPDRFTGSGSGTDFTLTISSVKAEDLAVYYCQQYYSYPLTFGAGTKLELK

SEQ ID NO: 515 - protein, 17 aa, Artificial Sequence
  ACI-8033-7A2-Ab1 VL CDR1
KSSQSLLYRSNQKNYLA

SEQ ID NO: 516 - protein, 7 aa, Artificial Sequence
  ACI-8033-7A2-Ab1 VL CDR2
WAFTRES

SEQ ID NO: 517 - protein, 9 aa, Artificial Sequence
  ACI-8033-7A2-Ab1 VL CDR3
QQYYSYPLT

SEQ ID NO: 518 - DNA, 360 bp, Artificial Sequence
  ACI-8033-7A2-Ab1 VH
caggtccaactacagcagcctgggactgaactggtgaagcctggggcttcagtgaacctgccctgcaaggcttctggctacaccttcaccagctactggatgcactgggtgaagcagaggcctggtcaaggccttgattggattggaaatgttaatcctaacaatagtgatagtaattacaatgagaagttcaagaggaaggccacactgactgtagacaaatcctccagcacagcctacatgcacctcagcagcctgacatctgaggactctgcggtctattattgtgcaagatctccttactacggtggccgttaccttgactactggggccaaggcaccactctcacagtctcctca

SEQ ID NO: 519 - DNA, 339 bp, Artificial Sequence
  ACI-8033-7A2-Ab1 VL
gacattgtgatgtcacagtctccatcctccctagctgtgtcagttggagagaaggttactatgacctgcaagtccagtcagagccttttatatagaagcaatcaaaagaactacttggcctggtaccagcagaaaccaggacagtctcctaaactgttgatttactgggcattcactagggaatctggggtccctgatcgcttcacaggcagtggatctgggacagatttcactctcaccatcagcagtgtgaaggctgaagacctggcagtttattactgtcagcaatattatagctatcctctcacgttcggtgctgggaccaagctggagctgaaa

SEQ ID NO: 520 - protein, 114 aa, Artificial Sequence
  ACI-8033-1A12-Ab1 VH
QVHLKQSGADLVRPGASVKLSCKASGYSFTDFYINWVKQTPGQGLEWIARIYPGNNNTFYNEKFKGKATLSAEKSSTTAYMQLSSLTSEDSAVYFCVVGYYGAWGQGTTLTVSS

SEQ ID NO: 521 - protein, 5 aa, Artificial Sequence
  ACI-8033-1A12-Ab1 VH CDR1
DFYIN

SEQ ID NO: 522 - protein, 17 aa, Artificial Sequence
  ACI-8033-1A12-Ab1 VH CDR2
RIYPGNNNTFYNEKFKG

SEQ ID NO: 523 - skipped

SEQ ID NO: 524 - protein, 112 aa, Artificial Sequence
  ACI-8033-1A12-Ab1 VL
DVVMTQTPLSLPVSLGDQASISCRSSQSLVHSNGNTHLHWYLQKPGQSPKLLIYKVSNRFSGVPDRFSGSGSGTDFTLKISRVEAEDLGFYFCSQSTHVPWTFGGGTKLEIK

SEQ ID NO: 525 - protein, 16 aa, Artificial Sequence
  ACI-8033-1A12-Ab1 VL CDR1
RSSQSLVHSNGNTHLH

SEQ ID NO: 526-527 - skipped (2 entries)

SEQ ID NO: 528 - DNA, 342 bp, Artificial Sequence
  ACI-8033-1A12-Ab1 VH
caggtccacctgaagcagtctggggctgacctggtgaggcctggggcttcagtgaagctgtcctgcaaggcgtctggctacagtttcactgacttttatataaattgggtgaagcagacgcctggacagggacttgagtggattgcgaggatttatcctggaaataataatactttctacaatgagaaattcaagggcaaggccacactgagtgcagaaaaatcctccaccactgcctacatgcagctcagcagcctgacatctgaggactctgctgtctatttctgtgtagtggggtactacggtgcctggggccaaggcaccactctcacagtctcctca

SEQ ID NO: 529 - DNA, 336 bp, Artificial Sequence
  ACI-8033-1A12-Ab1 VL
gatgttgtgatgacccaaactccactctccctgcctgtcagtcttggagatcaagcctccatctcttgcagatctagtcagagccttgtacacagtaatggaaacacccatttgcattggtacctgcagaagccaggccagtctccaaagctcctgatctataaagtttccaaccgattttctggggtcccagacaggttcagtggcagtggatcagggacagatttcacactcaagatcagcagagtggaggctgaggatctaggattttatttctgctctcaaagtacacatgttccgtggacgttcggtggaggcaccaagctggaaatcaaa

SEQ ID NO: 530 - protein, 125 aa, Artificial Sequence
  ACI-8033-4F3-Ab1 VH
EVQLQQSGPELVKPGASVKISCKASGYTFTDYYMNWVKQSHGKSLEWIGDINPNTGTNSYNQKFKGRASLTVDKFSSAAYMELRSLTSEDSAVYYCARTGYGDPISSYYYALDYWGQGTSVTVSS

SEQ ID NO: 531 - skipped

SEQ ID NO: 532 - protein, 17 aa, Artificial Sequence
  ACI-8033-4F3-Ab1 VH CDR2
DINPNTGTNSYNQKFKG

SEQ ID NO: 533 - protein, 16 aa, Artificial Sequence
  ACI-8033-4F3-Ab1 VH CDR3
TGYGDPISSYYYALDY

SEQ ID NO: 534 - protein, 112 aa, Artificial Sequence
  ACI-8033-4F3-Ab1 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEIK

SEQ ID NO: 535-536 - skipped (2 entries)

SEQ ID NO: 537 - protein, 8 aa, Artificial Sequence
  ACI-8033-4F3-Ab1 VL CDR3
KQSYNLWT

SEQ ID NO: 538 - DNA, 375 bp, Artificial Sequence
  ACI-8033-4F3-Ab1 VH
gaggtccagctgcaacaatctggacctgagctggtgaagcctggggcttcagtgaagatatcctgtaaggcttctggatacacgttcactgactactacatgaactgggtgaagcagagccatggaaagagccttgaatggattggagatattaatcctaacactggtactaatagctacaaccagaagttcaagggcagggcctcactgactgtagacaagttctccagcgcagcctacatggagctccgcagcctgacatctgaggactctgcagtctattactgtgcaagaaccggctatggcgaccctatttcctcatattactatgctctggactactggggtcaaggaacctcagtcaccgtctcctca

SEQ ID NO: 539 - DNA, 336 bp, Artificial Sequence
  ACI-8033-4F3-Ab1 VL
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtggacgttcggtggaggcaccaagctggaaatcaaa

SEQ ID NO: 540 - protein, 117 aa, Artificial Sequence
  ACI-8033-17F5-Ab1 VH
EVQLQQSGPELVKPGASVKISCKASGYTFTDYFMNWVKQSHGKSLEWIGDINPNIDVTNYNQKFKGKATLTVDKSSSTAYMELRSLTSEDSAVYYCARGRDYAMDFWGQGTSVTVSS

SEQ ID NO: 541 - skipped

SEQ ID NO: 542 - protein, 17 aa, Artificial Sequence
  ACI-8033-17F5-Ab1 VH CDR2
DINPNIDVTNYNQKFKG

SEQ ID NO: 543 - protein, 8 aa, Artificial Sequence
  ACI-8033-17F5-Ab1 VH CDR3
GRDYAMDF

SEQ ID NO: 544 - protein, 112 aa, Artificial Sequence
  ACI-8033-17F5-Ab1 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYDLWTFGGGTKLEIK

SEQ ID NO: 545-547 - skipped (3 entries)

SEQ ID NO: 548 - DNA, 351 bp, Artificial Sequence
  ACI-8033-17F5-Ab1 VH
gaggtccaactgcaacaatctggacctgagctggtgaagcctggggcttcagtgaagatatcctgtaaggcttctggatacacgttcactgactacttcatgaactgggtgaagcagagccatggaaagagccttgagtggattggagatattaatcctaacattgatgttactaactacaaccagaagttcaagggcaaggccacattgactgtagacaagtcctccagcacagcctacatggagctccgcagcctgacatctgaggactctgcagtctattactgtgcaagagggcgggactatgctatggacttctggggtcaaggaacctcagtcaccgtctcctca

SEQ ID NO: 549 - DNA, 336 bp, Artificial Sequence
  ACI-8033-17F5-Ab1 VL
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcaccctcaccatcagcagtgtgcaggctgaagacctggcagtttattactgcaagcaatcttatgatctgtggacgttcggtggaggcaccaagctggaaatcaaa

SEQ ID NO: 550 - protein, 116 aa, Artificial Sequence
  ACI-8033-18C11-Ab1 VH
QVQLQQPGAELVKPGASVKMSCKAAGYTFSSYWITWVRQRPGQGLDWIGDIYPGGGVTNYNEKFKTKATLTVDTSSSTAYMQLSSLTSEDSAVYYCATAQTTFAYWGQGTLVTVSA

SEQ ID NO: 551 - protein, 5 aa, Artificial Sequence
  ACI-8033-18C11-Ab1 VH CDR1
SYWIT

SEQ ID NO: 552 - protein, 17 aa, Artificial Sequence
  ACI-8033-18C11-Ab1 VH CDR2
DIYPGGGVTNYNEKFKT

SEQ ID NO: 553 - protein, 7 aa, Artificial Sequence
  ACI-8033-18C11-Ab1 VH CDR3
AQTTFAY

SEQ ID NO: 554 - protein, 112 aa, Artificial Sequence
  ACI-8033-18C11-Ab1 VL
DVLMTQTPLSLPVSLGDQASISCRSSQNIVHNNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 555 - protein, 16 aa, Artificial Sequence
  ACI-8033-18C11-Ab1 VL CDR1
RSSQNIVHNNGNTYLE

SEQ ID NO: 556 - skipped

SEQ ID NO: 557 - protein, 9 aa, Artificial Sequence
  ACI-8033-18C11-Ab1 VL CDR3
FQGSHVPRT

SEQ ID NO: 558 - DNA, 348 bp, Artificial Sequence
  ACI-8033-18C11-Ab1 VH
caggtccaactccagcagcctggggctgagcttgtgaagcctggggcttcagtgaagatgtcctgcaaggctgctggctacaccttcagcagctactggataacctgggtgaggcagaggcctggacaaggccttgactggattggagatatttatcctggtggaggtgttactaactacaatgagaagttcaagaccaaggccacactgactgtagacacatcctccagcacagcctacatgcagctcagcagcctgacatctgaggactctgcggtctattactgtgcgacagctcagactacgtttgcttactggggccaagggactctggtcactgtctctgca

SEQ ID NO: 559 - DNA, 336 bp, Artificial Sequence
  ACI-8033-18C11-Ab1 VL
gatgttttgatgacccaaactccactgtccctgcctgtcagtcttggagatcaagcctccatctcttgcagatctagtcagaacattgtacataataatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaa

SEQ ID NO: 560 - protein, 116 aa, Artificial Sequence
  ACI-8033-18D12-Ab1 VH
QVQLQQPGAELVKPGASVKMSCKASGYTFTSYWITWVRQRPGQGLDWIGDIYPGGGVTNYNEKFKTKATLTVDTSSSTAYMHLSSLTSEDSAVYFCATAQTTFAHWGQGTLVTVSA

SEQ ID NO: 561-562 - skipped (2 entries)

SEQ ID NO: 563 - protein, 7 aa, Artificial Sequence
  ACI-8033-18D12-Ab1 VH CDR3
AQTTFAH

SEQ ID NO: 564 - protein, 112 aa, Artificial Sequence
  ACI-8033-18D12-Ab1 VL
DVLMTQTPLSLPVSLGDQASISCRSSQNIAHNNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 565 - protein, 16 aa, Artificial Sequence
  ACI-8033-18D12-Ab1 VL CDR1
RSSQNIAHNNGNTYLE

SEQ ID NO: 566-567 - skipped (2 entries)

SEQ ID NO: 568 - DNA, 348 bp, Artificial Sequence
  ACI-8033-18D12-Ab1 VH
caggtccaactccagcagcctggggctgagcttgtgaagcctggggcttcagtgaagatgtcctgcaaggcttctggctacaccttcaccagctactggataacctgggtgaggcagaggcctggacaaggccttgactggattggagatatttatcctggtggaggtgttactaactacaatgagaagttcaagaccaaggccacactgactgtagacacatcctccagcacagcctacatgcacctcagcagcctgacatctgaggactctgcggtctatttctgtgcgacagctcagactacgtttgctcactggggccaagggactctggtcactgtctctgca

SEQ ID NO: 569 - DNA, 336 bp, Artificial Sequence
  ACI-8033-18D12-Ab1 VL
gatgttttgatgacccaaactccactctccctgcccgtcagtcttggagatcaagcctccatctcttgcagatctagtcagaatattgcacataataatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaa

SEQ ID NO: 570 - protein, 113 aa, Artificial Sequence
  ACI-8033-1F8-Ab1 VH
DVQLQESGPGLVKPSQSLSLTCSVTGYSITSGFYWNWIRQFPGNKLEWMGYISYDGSNNYNPSLKNRISIIRDTSKNQFFLKLKSVTSEDTATYYCVRGDVDWGQGTTLTVSS

SEQ ID NO: 571 - protein, 6 aa, Artificial Sequence
  ACI-8033-1F8-Ab1 VH CDR1
SGFYWN

SEQ ID NO: 572 - skipped

SEQ ID NO: 573 - protein, 4 aa, Artificial Sequence
  ACI-8033-1F8-Ab1 VH CDR3
GDVD

SEQ ID NO: 574 - protein, 112 aa, Artificial Sequence
  ACI-8033-1F8-Ab1 VL
DVVMTQTPLTLSVTIGQPASISCKSSQSLLDSDGETYLNWLFQRPGQSPKRLIYLVSKLDSGVPDRFTGSGSGTDFTLKISRVEPEDLGVYYCWQGTHFPQTLGGGTKLEIK

SEQ ID NO: 575-577 - skipped (3 entries)

SEQ ID NO: 578 - DNA, 339 bp, Artificial Sequence
  ACI-8033-1F8-Ab1 VH
gatgtacagcttcaggagtcaggacctggcctcgtgaaaccttctcagtctctgtctctcacctgctctgtcactggctactccatcaccagtgggttttactggaactggatccggcaatttccaggaaataaactggaatggatgggctacataagctacgatggtagcaataactacaacccatctctcaaaaatcgaatctccattattcgtgacacatctaagaaccagtttttcctgaagttgaaatctgtgacttctgaggacacagccacatattattgtgtaagaggggacgtcgactggggccaaggcaccactctcactgtctcctca

SEQ ID NO: 579 - DNA, 336 bp, Artificial Sequence
  ACI-8033-1F8-Ab1 VL
gatgttgtgatgacccagactccactcactttgtcggtcaccattggacaaccagcctccatctcttgcaagtcaagtcagagcctcttagatagtgatggagagacatatttgaattggttgtttcagaggccaggccagtctccaaagcgcctaatctatctggtgtctaaactggactctggagtccctgacaggttcactggcagtggatcagggacagatttcacactgaaaatcagcagagtggagcctgaggatttgggagtttattattgctggcaaggtacacattttcctcagacgctcggtggaggcaccaagctggaaatcaaa

SEQ ID NO: 580 - protein, 120 aa, Artificial Sequence
  ACI-8033-22E5-Ab1 VH
EVQLVESGGDLVKPGGSLKLSCAASGFTFSSYGMSWVRQTPDKRLEWVATISNGGSYTYYPDSVKGRFTISRDNAKNTLYLQMSSLKSEDTAMYYCARQLRRDGWYFDVWGTGTTVTVSS

SEQ ID NO: 581 - protein, 5 aa, Artificial Sequence
  ACI-8033-22E5-Ab1 VH CDR1
SYGMS

SEQ ID NO: 582 - protein, 17 aa, Artificial Sequence
  ACI-8033-22E5-Ab1 VH CDR2
TISNGGSYTYYPDSVKG

SEQ ID NO: 583 - protein, 11 aa, Artificial Sequence
  ACI-8033-22E5-Ab1 VH CDR3
QLRRDGWYFDV

SEQ ID NO: 584 - protein, 108 aa, Artificial Sequence
  ACI-8033-22E5-Ab1 VL
EIVLTQSPALMAASPGEKVTITCSVSSSISSSKLHWYQQKSETSPKLWIYGTSNLASGVPVRFSGSGSGTSYSLTISSMEAEDAATYYCQQWSSYPLTFGAGTKLELK

SEQ ID NO: 585 - protein, 12 aa, Artificial Sequence
  ACI-8033-22E5-Ab1 VL CDR1
SVSSSISSSKLH

SEQ ID NO: 586 - protein, 7 aa, Artificial Sequence
  ACI-8033-22E5-Ab1 VL CDR2
GTSNLAS

SEQ ID NO: 587 - protein, 9 aa, Artificial Sequence
  ACI-8033-22E5-Ab1 VL CDR3
QQWSSYPLT

SEQ ID NO: 588 - DNA, 360 bp, Artificial Sequence
  ACI-8033-22E5-Ab1 VH
gaggtgcaactagtggagtctgggggagacttagtgaagcctggagggtccctgaaactctcctgtgcagcctctggattcactttcagtagctatggcatgtcttgggttcgccagactccagacaagaggctggagtgggtcgcaaccattagtaatggtggtagttacacctactatccagacagtgtgaaggggcgattcaccatctccagagacaatgccaagaacaccctgtacctgcaaatgagcagtctgaagtctgaggacacagccatgtattactgtgcaagacaattacgacgggacggttggtacttcgatgtctggggcacagggaccacggtcaccgtctcctca

SEQ ID NO: 589 - DNA, 324 bp, Artificial Sequence
  ACI-8033-22E5-Ab1 VL
gaaattgtgctcacccagtctccagcactcatggctgcatctccaggggagaaggtcaccatcacctgcagtgtcagctcaagtataagttccagcaagttgcactggtaccagcagaagtcagaaacctcccccaaactctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaa

SEQ ID NO: 590 - protein, 115 aa, Artificial Sequence
  ACI-8033-27D8-Ab1 VH
EVQLVESGGGLVQPKGSLKLSCAASGFTFNTYAMNWVRQAPGKGLEWVARIRSKSNNFATYYADSVKDRFTISRDESESMLYLQMNNLKAEDTAMYYCVRSFDYWGQGTTLTVSS

SEQ ID NO: 591-597 - skipped (7 entries)

SEQ ID NO: 598 - DNA, 345 bp, Artificial Sequence
  ACI-8033-27D8-Ab1 VH
gaggtgcagcttgttgagtctggtggaggattggtgcagcctaaagggtcattgaaactctcatgtgcagcctctggattcaccttcaatacctacgccatgaactgggtccgccaggctccaggaaagggtttggaatgggttgctcgcataagaagtaaaagtaataattttgcaacatattatgccgattcagtgaaagacagattcaccatctccagagatgaatcagaaagcatgctctatctgcaaatgaacaacttgaaagctgaggacacagccatgtattactgtgtgaggtcctttgactactggggccaaggcaccactctcacagtctcctca

SEQ ID NO: 599 - skipped

SEQ ID NO: 600 - protein, 116 aa, Artificial Sequence
  ACI-8033-21C8-Ab1 VH
QVQLQQPGAELVKPGASVKMSCKASGYTFTSYWITWVRQRPGQGLDWIGDIYPGGGVTNYNEKFKTKATLTVDTSSSTAYMHLSSLTSEDSAVYFCATAQTTFAYWGQGTLVTVSA

SEQ ID NO: 601-607 - skipped (7 entries)

SEQ ID NO: 608 - DNA, 348 bp, Artificial Sequence
  ACI-8033-21C8-Ab1 VH
caggtccaactccagcagcctggggctgagcttgtgaagcctggggcttcagtgaagatgtcctgcaaggcttctggctacaccttcaccagctactggataacctgggtgaggcagaggcctggacaaggccttgactggattggagatatttatcctggtggaggtgttactaactacaatgagaagttcaagaccaaggccacactgactgtagacacatcctccagcacagcctacatgcacctcagcagcctgacatctgaggactctgcggtctatttctgtgcgacagctcagactacgtttgcttactggggccaagggactctggtcactgtctctgca

SEQ ID NO: 609 - DNA, 336 bp, Artificial Sequence
  ACI-8033-21C8-Ab1 VL
gatgttttgatgacccaaactccactctccctgcctgtcagtcttggagatcaagcctccatctcttgcagatctagtcagaacattgtacataataatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaa

SEQ ID NO: 610 - protein, 119 aa, Artificial Sequence
  ACI-7079-3101E3-Ab1 VH
QVQLKESGPGLVAPSQSLSITCTVSGFSLTRYIINWVRQPPGKGLEWLGVIWTGGGTDYNSALKSRLSISKDNSKSQVFLKMNSLQTDDTARYYCAREYGYDGAWFAYWGQGTLVTVSA

SEQ ID NO: 611 - protein, 5 aa, Artificial Sequence
  ACI-7079-3101E3-Ab1 VH CDR1
RYIIN

SEQ ID NO: 612 - protein, 16 aa, Artificial Sequence
  ACI-7079-3101E3-Ab1 VH CDR2
VIWTGGGTDYNSALKS

SEQ ID NO: 613 - protein, 11 aa, Artificial Sequence
  ACI-7079-3101E3-Ab1 VH CDR3
EYGYDGAWFAY

SEQ ID NO: 614 - protein, 112 aa, Artificial Sequence
  ACI-7079-3101E3-Ab1 VL
DVLMTQTPLSLPVSLGDQASISCRSSQTIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 615 - protein, 16 aa, Artificial Sequence
  ACI-7079-3101E3-Ab1 VL CDR1
RSSQTIVHSNGNTYLE

SEQ ID NO: 616 - protein, 7 aa, Artificial Sequence
  ACI-7079-3101E3-Ab1 VL CDR2
KVSNRFS

SEQ ID NO: 617 - protein, 9 aa, Artificial Sequence
  ACI-7079-3101E3-Ab1 VL CDR3
FQGSHVPYT

SEQ ID NO: 618 - DNA, 357 bp, Artificial Sequence
  ACI-7079-3101E3-Ab1 VH
caggtgcagctgaaggagtcaggacctggcctggtggcgccctcacagagcctgtccatcacatgcactgtctctggcttctcattaaccaggtatattataaattgggttcgccagccaccaggaaagggtctggagtggcttggagtaatctggactggtggaggcacagattataattcagctctcaaatccagactgagcatcagcaaagacaactccaagagtcaagttttcttaaaaatgaacagtctgcaaactgatgacacagccaggtactactgtgccagagagtatggttacgacggggcctggtttgcttactggggccaagggactcttgtcactgtctctgca

SEQ ID NO: 619 - DNA, 336 bp, Artificial Sequence
  ACI-7079-3101E3-Ab1 VL
gatgttttgatgacccaaactccactctccctgcctgtcagtcttggagatcaagcctccatctcttgcagatctagtcagaccattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 620 - protein, 123 aa, Artificial Sequence
  ACI-7079-3103D9-Ab1 VH
QVSLKESGPGILQPSQTLSLTCSFSGFSLSTFGMGVGWIRQPSGKGLDWLTHIWWDDDKYYNPGLKSRLTISKDTSKNQVFLEIANVDTADTATYYCARIGDYYGTRGYFDVWGTGTTVTVSS

SEQ ID NO: 621 - protein, 7 aa, Artificial Sequence
  ACI-7079-3103D9-Ab1 VH CDR1
TFGMGVG

SEQ ID NO: 622 - protein, 16 aa, Artificial Sequence
  ACI-7079-3103D9-Ab1 VH CDR2
HIWWDDDKYYNPGLKS

SEQ ID NO: 623 - protein, 13 aa, Artificial Sequence
  ACI-7079-3103D9-Ab1 VH CDR3
IGDYYGTRGYFDV

SEQ ID NO: 624 - protein, 113 aa, Artificial Sequence
  ACI-7079-3103D9-Ab1 VL
DIVMTQSPSSLTMSLGQTVTMSCKSSQSLLNSSNQKNHLAWYQQRPGQSPKLLVYFASTRESGVPERFIGGGSGTDFTLTISNVQSEDLTDYFCQQHYSTPYTFGGGTKLEIK

SEQ ID NO: 625 - protein, 17 aa, Artificial Sequence
  ACI-7079-3103D9-Ab1 VL CDR1
KSSQSLLNSSNQKNHLA

SEQ ID NO: 626 - protein, 7 aa, Artificial Sequence
  ACI-7079-3103D9-Ab1 VL CDR2
FASTRES

SEQ ID NO: 627 - protein, 9 aa, Artificial Sequence
  ACI-7079-3103D9-Ab1 VL CDR3
QQHYSTPYT

SEQ ID NO: 628 - DNA, 369 bp, Artificial Sequence
  ACI-7079-3103D9-Ab1 VH
caggtttctctgaaagagtctggccctgggatattgcagccctcccagaccctcagtctgacttgctctttctctgggttttcactgagcacttttggtatgggtgtaggctggattcgtcagccttcagggaagggtctggactggctgacacacatttggtgggatgatgataagtactataatccaggcctgaagagtcgcctcacaatctccaaggatacctccaaaaaccaggtattcctcgagatcgccaatgtggacactgcagatactgccacatattactgtgcgcgaataggagattactacggtactagagggtacttcgatgtctggggcacagggaccacggtcaccgtctcctca

SEQ ID NO: 629 - DNA, 339 bp, Artificial Sequence
  ACI-7079-3103D9-Ab1 VL
gacattgtgatgacacagtctccatcctccctgactatgtcactaggacagacggtcactatgagctgcaagtccagtcagagccttttaaatagtagcaatcaaaagaaccatctggcctggtaccagcagagaccaggacagtctcccaaacttctggtatactttgcatccactagggaatctggggtccctgagcgcttcataggcggtggatctgggacagatttcactcttaccatcagcaatgtgcagtctgaagacctgacagattacttctgtcagcaacattatagcactccgtacacgttcggaggggggaccaagctggaaataaaa

SEQ ID NO: 630 - protein, 118 aa, Artificial Sequence
  ACI-7079-3103G12-Ab2 VH
DVQLQESGPGMVKPSQSLSLTCTVTGYSITSGYDWHWIRHFPGNKLEWMGYITYSGSTNYNPSLKSRISITHDTSKNHFFLKLTSVTTEDTATYYCARDTVDAWFAYWGQGTLVTVSA

SEQ ID NO: 631 - protein, 6 aa, Artificial Sequence
  ACI-7079-3103G12-Ab2 VH CDR1
SGYDWH

SEQ ID NO: 632 - protein, 16 aa, Artificial Sequence
  ACI-7079-3103G12-Ab2 VH CDR2
YITYSGSTNYNPSLKS

SEQ ID NO: 633 - protein, 9 aa, Artificial Sequence
  ACI-7079-3103G12-Ab2 VH CDR3
DTVDAWFAY

SEQ ID NO: 634 - protein, 112 aa, Artificial Sequence
  ACI-7079-3103G12-Ab2 VL
DVLMTQAPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPPTFGGGTKLEIK

SEQ ID NO: 635 - protein, 16 aa, Artificial Sequence
  ACI-7079-3103G12-Ab2 VL CDR1
RSSQSIVHSNGDTYLE

SEQ ID NO: 636 - skipped

SEQ ID NO: 637 - protein, 9 aa, Artificial Sequence
  ACI-7079-3103G12-Ab2 VL CDR3
FQGSHVPPT

SEQ ID NO: 638 - DNA, 354 bp, Artificial Sequence
  ACI-7079-3103G12-Ab2 VH
gatgtgcagcttcaggagtcaggacctggcatggtgaaaccttctcagtcactttccctcacctgcactgtcactggctactccatcaccagtggttatgactggcactggatccgacattttccaggaaacaaactggagtggatgggctacataacctacagtggtagcactaactacaacccatccctcaaaagtcgaatctccatcactcatgacacatctaagaaccatttcttcctgaagttgacttctgtgactactgaggacacagccacatattactgtgcaagagacacggtagacgcctggtttgcttactggggccaggggactctggtcactgtctctgca

SEQ ID NO: 639 - DNA, 336 bp, Artificial Sequence
  ACI-7079-3103G12-Ab2 VL
gatgttttgatgacccaagctccactctccctgcctgtcagtcttggagatcaagcctccatctcgtgcagatctagtcagagcattgtacatagtaatggagacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 640 - protein, 123 aa, Artificial Sequence
  ACI-7079-3104F12-Ab2 VH
QVTLKESGPGIVQPSQTLSLTCSFSGFSLNTFGLGVGWIRQPSGKGLDWLTHIWWDDDKYYNPALKSRLTISKDTSKNQVFLKIANVDTADTATYYCARIGDFYGSRGYFDVWGTGTTVTVSS

SEQ ID NO: 641 - protein, 7 aa, Artificial Sequence
  ACI-7079-3104F12-Ab2 VH CDR1
TFGLGVG

SEQ ID NO: 642 - protein, 16 aa, Artificial Sequence
  ACI-7079-3104F12-Ab2 VH CDR2
HIWWDDDKYYNPALKS

SEQ ID NO: 643 - protein, 13 aa, Artificial Sequence
  ACI-7079-3104F12-Ab2 VH CDR3
IGDFYGSRGYFDV

SEQ ID NO: 644 - protein, 113 aa, Artificial Sequence
  ACI-7079-3104F12-Ab2 VL
DIVMTQSPSSLAMSVGQKVTLRCKSSQSLLNSSNQKNHLAWYQQKPGQSPKLLLCFASTRESGVPDRFIGSGSGTDFTLTISSVQAEDLADYFCQQHYSTPYTFGGGTKLEIK

SEQ ID NO: 645-647 - skipped (3 entries)

SEQ ID NO: 648 - DNA, 369 bp, Artificial Sequence
  ACI-7079-3104F12-Ab2 VH
caggttactctgaaagagtctggccctggaatagtgcagccctcccagaccctcagtctgacttgctctttctctggattttcactgaacacttttggtttgggtgtaggctggattcgtcagccttcagggaagggtctggactggctgacacacatttggtgggatgatgataagtactataatccagccctgaagagtcggctcacaatctccaaggatacctccaaaaaccaggtattcctcaagatcgccaatgtggacactgcagatactgccacatactactgtgctcgaataggcgatttctacggtagtagaggatatttcgatgtctggggcacagggaccacggtcaccgtctcctca

SEQ ID NO: 649 - DNA, 339 bp, Artificial Sequence
  ACI-7079-3104F12-Ab2 VL
gacattgtgatgacacagtctccatcctccctggctatgtcagttggacagaaggtcactttgaggtgcaagtccagtcagagccttttaaatagtagtaatcaaaagaaccatttggcctggtaccagcagaaaccaggacagtctcctaaacttctgctatgctttgcatccactagggaatctggggtccctgatcgcttcataggcagtggatctgggacagatttcactcttaccatcagtagtgtgcaggctgaagacctggcagattacttctgtcagcaacattatagcactccgtacacgttcggaggggggaccaagctggaaataaaa

SEQ ID NO: 650 - protein, 123 aa, Artificial Sequence
  ACI-7079-3106C5-Ab1 VH
QVTLKESGPGILQPSQTLSLTCSFSGFSLSTFGMGVGWIRQPSGKGLEWLTHIWWDDDKYYNPALKSRLTISRDPSKNQVFLKIANVDTADTATYYCARIGDYYGSRGYFDVWGTGTTVTVSS

SEQ ID NO: 651-652 - skipped (2 entries)

SEQ ID NO: 653 - protein, 13 aa, Artificial Sequence
  ACI-7079-3106C5-Ab1 VH CDR3
IGDYYGSRGYFDV

SEQ ID NO: 654 - protein, 113 aa, Artificial Sequence
  ACI-7079-3106C5-Ab1 VL
DIVMTQSPSSLAMSVGQKVTMSCKSRQSLLNSSNQKNHLAWYQQKPGQSPKLLVYFASTRESGVPDRFIGSGSGTDFTLTISSVQAEDLADYFCQQHYSTPYTFGGGTKLEIK

SEQ ID NO: 655 - protein, 17 aa, Artificial Sequence
  ACI-7079-3106C5-Ab1 VL CDR1
KSRQSLLNSSNQKNHLA

SEQ ID NO: 656-657 - skipped (2 entries)

SEQ ID NO: 658 - DNA, 368 bp, Artificial Sequence
  ACI-7079-3106C5-Ab1 VH
caggttactctgaaagagtctggccctgggatattgcagccctcccagaccctcagtctgacttgttctttctctgggttttcactgagtacttttggtatgggtgtaggctggattcgtcagccttcagggaagggtctggagtggctaacacacatttggtgggatgatgataagtactataacccagccctgaagagtcggctcacaatctccagggatccctccaaaaaccaggtattctcaagatcgccaatgtggacactgcagatactgccacatactactgtgctcgaataggggattactacggtagtagaggatacttcgatgtctggggcacagggaccacggtcaccgtctcctca

SEQ ID NO: 659 - DNA, 339 bp, Artificial Sequence
  ACI-7079-3106C5-Ab1
gacattgtgatgacacagtctccatcctccctggctatgtcagtaggacagaaggtcactatgagctgcaagtccaggcagagccttttaaatagtagtaatcaaaagaatcatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattatagcactccgtacacgttcgggggggggaccaagctggaaataaaa

SEQ ID NO: 660 - protein, 119 aa, Artificial Sequence
  ACI-7079-3106F2-Ab1 VH
EVQLQQSGAELVRPGASVKLSCTASGFNVKDDYMHWVKQRPEQGLEWIGWIDPENGDTEYASKFQGKATLTADTSSNTAYLQLSGLTSVDTAVYYCTTAGTSPYYFDYWGQGTTLTVSS

SEQ ID NO: 661 - protein, 5 aa, Artificial Sequence
  ACI-7079-3106F2-Ab1 VH CDR1
DDYMH

SEQ ID NO: 662 - protein, 17 aa, Artificial Sequence
  ACI-7079-3106F2-Ab1 VH CDR2
WIDPENGDTEYASKFQG

SEQ ID NO: 663 - protein, 10 aa, Artificial Sequence
  ACI-7079-3106F2-Ab1 VH CDR3
AGTSPYYFDY

SEQ ID NO: 664 - protein, 107 aa, Artificial Sequence
  ACI-7079-3106F2-Ab1 VL
DIVMTQSQKFMSTSVGDRVSVTCKASQNVGTNVAWYQQKVGQSPKALIYSASYRYSGVPDRFTGSGSGTDFTLTISNVQSEDLADYFCQQYNSYPLTFGSGTKLEIK

SEQ ID NO: 665 - protein, 11 aa, Artificial Sequence
  ACI-7079-3106F2-Ab1 VL CDR1
KASQNVGTNVA

SEQ ID NO: 666 - protein, 7 aa, Artificial Sequence
  ACI-7079-3106F2-Ab1 VL CDR2
SASYRYS

SEQ ID NO: 667 - protein, 9 aa, Artificial Sequence
  ACI-7079-3106F2-Ab1 VL CDR3
QQYNSYPLT

SEQ ID NO: 668 - DNA, 357 bp, Artificial Sequence
  ACI-7079-3106F2-Ab1 VH
gaggttcagctgcagcagtctggggctgagcttgtgaggccaggggcctcagtcaagttgtcctgcacagcttctggctttaacgttaaagacgactatatgcactgggtgaagcagaggcctgaacagggcctggagtggattggatggattgatcctgagaatggtgatactgaatatgcctcgaagttccagggcaaggccactttaacagcagacacatcctccaacacagcctacctgcagctcagcggcctgacatctgtggacactgccgtctattactgtactacagccggtactagcccgtactactttgactactggggccaaggcaccactctcacagtctcctcc

SEQ ID NO: 669 - DNA, 321 bp, Artificial Sequence
  ACI-7079-3106F2-Ab1 VL
gacattgtgatgacccagtctcaaaaattcatgtccacatcagtaggagacagggtcagcgtcacctgcaaggccagtcagaatgtgggaactaatgtagcctggtatcaacagaaagtaggacaatctcctaaagcactgatttactcggcatcctaccggtacagtggagtccctgatcgcttcacaggcagtggatctgggacagatttcactctcaccatcagcaatgtgcagtctgaagacttggcagactatttctgtcagcaatataacagctatcctctcacgttcggctcggggacaaagttggaaataaaa

SEQ ID NO: 670 - protein, 117 aa, Artificial Sequence
  ACI-7079-3112H1-Ab1 VH
EVQLQQSGPELVKPGASVKISCKASGYTFSDYFMNWVKQSHGKSLEWIGDINPNNGGSSYIQKFKGKATLTVDKSSTTAYMELRSLTSEDSAVYYCARGSNYAMDSWGQGTSVTVSS

SEQ ID NO: 671 - protein, 5 aa, Artificial Sequence
  ACI-7079-3112H1-Ab1 VH CDR1
DYFMN

SEQ ID NO: 672 - protein, 17 aa, Artificial Sequence
  ACI-7079-3112H1-Ab1 VH CDR2
DINPNNGGSSYIQKFKG

SEQ ID NO: 673 - protein, 8 aa, Artificial Sequence
  ACI-7079-3112H1-Ab1 VH CDR3
GSNYAMDS

SEQ ID NO: 674 - protein, 112 aa, Artificial Sequence; identical to SEQ ID NO: 544
  ACI-7079-3112H1-Ab1 VL

SEQ ID NO: 675 - protein, 17 aa, Artificial Sequence
  ACI-7079-3112H1-Ab1 VL CDR1
KSSQSLLNSRTRKNYLA

SEQ ID NO: 676 - protein, 7 aa, Artificial Sequence
  ACI-7079-3112H1-Ab1 VL CDR2
WASTRES

SEQ ID NO: 677 - protein, 8 aa, Artificial Sequence
  ACI-7079-3112H1-Ab1 VL CDR3
KQSYDLWT

SEQ ID NO: 678 - DNA, 351 bp, Artificial Sequence
  ACI-7079-3112H1-Ab1 VH
gaggtccagctgcaacaatctggacctgaactggtgaagcctggggcttcagtgaagatatcctgtaaggcttctggatacacgttcagtgactacttcatgaactgggtgaagcagagtcatggaaaaagccttgagtggattggagatattaatcctaacaatggtggttctagctacatccagaagttcaagggcaaggccacattgactgtagacaagtcctccaccacagcctatatggagctccgcagcctgacatctgaggactcagcagtctattactgtgcaagaggtagcaactatgctatggactcctggggtcagggaacctcagtcaccgtctcctca

SEQ ID NO: 679 - DNA, 336 bp, Artificial Sequence
  ACI-7079-3112H1-Ab1 VL
gacattgtgatgtcacagtctccatcctccctggctgtgtcagcaggagagaaggtcactatgagctgcaaatccagtcagagtctgctcaacagtagaacccggaagaactacttggcttggtaccagcagaaaccaggacagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttatgatctgtggacgttcggtgggggcaccaagctggaaatcaaa

SEQ ID NO: 680 - protein, 123 aa, Artificial Sequence
  ACI-7079-3107E6-Ab1 VH
QVTLKESGPGILQPSQTLSLTCSFSGFSLSTFGMGVGWIRQPSGKGLEWLTHIWWDDDKYYNPALKSRLTISKDASKNQMFLKIANVDTADTATYFCARIGDYYGSRGFFDVWGTGTTVTVSS

SEQ ID NO: 681-682 - skipped (2 entries)

SEQ ID NO: 683 - protein, 13 aa, Artificial Sequence
  ACI-7079-3107E6-Ab1 VH CDR3
IGDYYGSRGFFDV

SEQ ID NO: 684 - protein, 113 aa, Artificial Sequence
  ACI-7079-3107E6-Ab1 VL
DIVMTQSPSSLAMSVGQKVTMSCKSSQSLLNSSNQKNHLAWYQQKPGQSPKLLVYFASTRDSGVPDRFIGSGSGTDFTLTISSVQAEDLADYFCQQHYSTPYTFGGGTKLEIK

SEQ ID NO: 685 - skipped

SEQ ID NO: 686 - protein, 7 aa, Artificial Sequence
  ACI-7079-3107E6-Ab1 VL CDR2
FASTRDS

SEQ ID NO: 687 - protein, 9 aa, Artificial Sequence
  ACI-7079-3107E6-Ab1 VL CDR3
QQHYSTPYT

SEQ ID NO: 688 - DNA, 369 bp, Artificial Sequence
  ACI-7079-3107E6-Ab1 VH
caggttactctgaaagagtctggccctgggatattgcagccctcccagaccctcagtctgacttgttctttctctgggttttcactgagcacttttggtatgggtgtaggctggattcgtcagccttcagggaagggtctggagtggctgacacacatttggtgggatgatgataaatactataacccagcccttaagagtcggctcacaatctccaaggatgcctccaaaaaccagatgttcctcaagatcgccaatgtggacactgcagatactgccacatacttctgtgctcgaataggggattactacggtagtagagggttcttcgatgtctggggcacagggaccacggtcaccgtctcctca

SEQ ID NO: 689 - DNA, 339 bp, Artificial Sequence
  ACI-7079-3107E6-Ab1 VL
gacattgtgatgacacagtctccatcctccctggccatgtcagtaggacagaaggtcactatgagctgcaagtccagtcagagccttttaaatagtagcaatcaaaagaaccatttggcctggtatcagcagaaaccaggacagtctcctaaacttctggtatactttgcatccactagggactctggggtccctgatcgcttcataggcagtggatctgggacagatttcactcttaccatcagcagtgtgcaggctgaggacctggcagattacttctgtcagcaacattatagcactccgtacacgttcggaggggggaccaagctggaaataaaa

SEQ ID NO: 690 - protein, 114 aa, Artificial Sequence
  ACI-7079-3108C10-Ab2 VH
ELQLVESGGGLVRPGGSLKLSCATSGFTFRNYAMSWVRQTPAKRLEWVATISDGGTYTFYSDNVKGRFTISRDNVKNTLYLQMSHLKSEDTAMYYCSRAGSGHWGQGTTLTVSS

SEQ ID NO: 691 - protein, 5 aa, Artificial Sequence
  ACI-7079-3108C10-Ab2 VH CDR1
NYAMS

SEQ ID NO: 692 - protein, 17 aa, Artificial Sequence
  ACI-7079-3108C10-Ab2 VH CDR2
TISDGGTYTFYSDNVKG

SEQ ID NO: 693 - protein, 5 aa, Artificial Sequence
  ACI-7079-3108C10-Ab2 VH CDR3
AGSGH

SEQ ID NO: 694 - protein, 112 aa, Artificial Sequence
  ACI-7079-3108C10-Ab2 VL
DVVMTQTPRTLSVIIGQPASISCKSSQSLLDSDGKTYLSWLLQRPGQSPKRLIYLVSKVDSGVPDRFTGSGSGTDFTLKISRVEAEDLGVYYCWQGTYFPRTFGGGTKLEIK

SEQ ID NO: 695 - protein, 16 aa, Artificial Sequence
  ACI-7079-3108C10-Ab2 VL CDR1
KSSQSLLDSDGKTYLS

SEQ ID NO: 696 - protein, 7 aa, Artificial Sequence
  ACI-7079-3108C10-Ab2 VL CDR2
LVSKVDS

SEQ ID NO: 697 - protein, 9 aa, Artificial Sequence
  ACI-7079-3108C10-Ab2 VL CDR3
WQGTYFPRT

SEQ ID NO: 698 - DNA, 342 bp, Artificial Sequence
  ACI-7079-3108C10-Ab2 VH
gaactgcagctggtggagtctgggggaggcttagtgaggcctggagggtccctgaaactctcctgtgcaacctctggattcactttcagaaactatgccatgtcttgggttcgccagactccggcaaagaggctggagtgggtcgcaaccattagtgatggtggtacttacaccttctattcagacaatgtaaagggccgattcaccatctccagagacaatgtcaagaacaccttgtaccttcaaatgagccatctgaagtctgaggacacagccatgtattactgttcaagagccggctctggccactggggccaaggcaccactctcacagtctcctca

SEQ ID NO: 699 - DNA, 336 bp, Artificial Sequence
  ACI-7079-3108C10-Ab2 VL
gatgttgtgatgacccagactccacgcactttgtcggtaatcattggacaaccagcctccatctcttgcaagtcaagtcagagcctcttagatagtgatggaaagacatacttgagttggttgttacagaggccaggccagtctccaaagcgcctaatctatctggtgtctaaagtggactctggagtccctgacaggttcactggcagtggatcagggacagatttcacactgaaaatcagcagagtggaggctgaggatttgggagtttattattgctggcaaggtacatattttcctcggacgttcggtggaggcaccaagctggaaatcaaa

SEQ ID NO: 700 - protein, 117 aa, Artificial Sequence
  ACI-8030-6106F5-Ab1 VH
QVQLQQSGPELVKPGASVKISCKASGYSFTTYYIHWMKQRPGQGPEWIGWIYPGRGYTKYNEKFKDKAAQTADTSSNTAYMQLSSLRSEDSAVYYCARDWETGFPYWGQGTLVTVSA

SEQ ID NO: 701 - protein, 5 aa, Artificial Sequence
  ACI-8030-6106F5-Ab1 VH CDR1
TYYIH

SEQ ID NO: 702 - protein, 17 aa, Artificial Sequence
  ACI-8030-6106F5-Ab1 VH CDR2
WIYPGRGYTKYNEKFKD

SEQ ID NO: 703 - protein, 8 aa, Artificial Sequence
  ACI-8030-6106F5-Ab1 VH CDR3
DWETGFPY

SEQ ID NO: 704 - protein, 111 aa, Artificial Sequence
  ACI-8030-6106F5-Ab1 VL
DIVLTQSPASLAVSLGQRATISCKASQSVDYGGDSWLNWYQQKPGQPPKLLIYAASNLDSGIPARFGGSGSGTDFTLNIHPVEEEDAATYYCQQSGEDPFTFGGGTNLEIK

SEQ ID NO: 705 - protein, 15 aa, Artificial Sequence
  ACI-8030-6106F5-Ab1 VL CDR1
KASQSVDYGGDSWLN

SEQ ID NO: 706 - protein, 7 aa, Artificial Sequence
  ACI-8030-6106F5-Ab1 VL CDR2
AASNLDS

SEQ ID NO: 707 - protein, 9 aa, Artificial Sequence
  ACI-8030-6106F5-Ab1 VL CDR3
QQSGEDPFT

SEQ ID NO: 708 - DNA, 351 bp, Artificial Sequence
  ACI-8030-6106F5-Ab1 VH
caggtccagctgcagcagtctggacctgaactggtgaagcctggggcttcagtgaagatatcctgcaaggcttctggctacagcttcacaacctactatatacactggatgaagcagaggcctggacagggacctgagtggattggatggatttatcctggaaggggttatactaagtacaatgagaagttcaaggacaaggccgcacagacggcagacacatcctccaacactgcctacatgcagctcagcagcctaagatctgaggactctgcggtctattactgtgcaagagactgggaaaccggatttccttactggggccaaggtactctggtcactgtctctgca

SEQ ID NO: 709 - DNA, 333 bp, Artificial Sequence
  ACI-8030-6106F5-Ab1 VL
gacattgtgctgacccaatctccagcttctttggctgtgtctctagggcagagggccaccatctcctgcaaggccagccaaagtgttgattatggtggtgatagttggctgaactggtaccaacagaaaccaggacagccacccaaactcctcatctatgctgcatccaatctagattctgggatcccagccaggtttggtggcagtgggtctgggacagacttcaccctcaacatccatcctgtggaggaggaggatgctgcaacctattactgtcaacaaagtggagaggatccgttcacgttcggaggggggaccaatctggaaataaaa

SEQ ID NO: 710 - protein, 114 aa, Artificial Sequence
  ACI-8031-6207G10-Ab1 VH
QVQLQQSGAELVKPGASVKISCKASGYAFSSYWMNWVKQRPGKGLEWIGQIYPGDGDTNYNGKFKGKATLTADRSSSTAYMQLSSLTSEDSAVYFCAITHYDYWGQGTTLTVSS

SEQ ID NO: 711 - protein, 5 aa, Artificial Sequence
  ACI-8031-6207G10-Ab1 VH CDR1
SYWMN

SEQ ID NO: 712 - protein, 17 aa, Artificial Sequence
  ACI-8031-6207G10-Ab1 VH CDR2
QIYPGDGDTNYNGKFKG

SEQ ID NO: 713 - protein, 5 aa, Artificial Sequence
  ACI-8031-6207G10-Ab1 VH CDR3
THYDY

SEQ ID NO: 714 - protein, 107 aa, Artificial Sequence
  ACI-8031-6207G10-Ab1 VL
DILLTQSPAILSVSPGDRVSFSCRASQSIGTRIHWYQQRTNGSPRLLIKYASESISGTPSRFSGSGSGTDFTLSINSVESEDIADYYCQQSNSWPLTFGAGTKLELK

SEQ ID NO: 715 - protein, 11 aa, Artificial Sequence
  ACI-8031-6207G10-Ab1 VL CDR1
RASQSIGTRIH

SEQ ID NO: 716 - protein, 7 aa, Artificial Sequence
  ACI-8031-6207G10-Ab1 VL CDR2
YASESIS

SEQ ID NO: 717 - protein, 9 aa, Artificial Sequence
  ACI-8031-6207G10-Ab1 VL CDR3
QQSNSWPLT

SEQ ID NO: 718 - DNA, 342 bp, Artificial Sequence
  ACI-8031-6207G10-Ab1 VH
caggttcagctgcagcagtctggggctgagctggtgaagcctggggcctcagtgaagatttcctgcaaagcttctggctacgcattcagtagctactggatgaactgggtgaagcagaggcctggaaagggtcttgagtggattggacagatttatcctggagatggtgatactaactacaacggaaagttcaagggcaaggccacactgactgcagacagatcctccagcacagcctacatgcagctcagcagcctgacctctgaggactctgcggtctatttctgtgcaattacgcactatgactactggggccaaggcaccactctcacagtctcctca

SEQ ID NO: 719 - DNA, 321 bp, Artificial Sequence
  ACI-8031-6207G10-Ab1 VL
gacatcttgctgactcagtctccagccatcctgtctgtgagtccaggagatagagtcagtttctcctgcagggccagtcagagcattggcacaaggatacactggtatcagcaaagaacaaatggttctccaaggcttctcataaagtatgcttctgagtctatctctgggaccccttccaggtttagtggcagtggatcagggacagattttactcttagcatcaacagtgtggagtctgaagatattgcagattattactgtcaacaaagtaatagttggccactcacgttcggtgctgggaccaagctggagctgaaa

SEQ ID NO: 720 - protein, 122 aa, Artificial Sequence
  ACI-8032-6301A10-Ab2 VH
QVQLQQSGAELARPGASVKLSCKASGYTFTSYGISWVKQRTGQGLEWIGEIYPRSGNTYYNEKFKGKATLTADKSSSTAYMELRSLTSEDSAVYFCASESYYGDYDWYFDVWGTGTTVTVSS

SEQ ID NO: 721 - protein, 5 aa, Artificial Sequence
  ACI-8032-6301A10-Ab2 VH CDR1
SYGIS

SEQ ID NO: 722 - protein, 17 aa, Artificial Sequence
  ACI-8032-6301A10-Ab2 VH CDR2
EIYPRSGNTYYNEKFKG

SEQ ID NO: 723 - protein, 13 aa, Artificial Sequence
  ACI-8032-6301A10-Ab2 VH CDR3
ESYYGDYDWYFDV

SEQ ID NO: 724 - protein, 112 aa, Artificial Sequence
  ACI-8032-6301A10-Ab2 VL
DVLMTQTPLSLPVSLGDQASISCRSSHSIEHSNGNTYLEWCLQKPGQPPKFLIYKVSNRFSGVPDRFSGSGSGTDFTLRISRVEAEDLGVYYCFQGSHVPPTFGGGTKLEIK

SEQ ID NO: 725 - protein, 16 aa, Artificial Sequence
  ACI-8032-6301A10-Ab2 VL CDR1
RSSHSIEHSNGNTYLE

SEQ ID NO: 726-727 - skipped (2 entries)

SEQ ID NO: 728 - DNA, 366 bp, Artificial Sequence
  ACI-8032-6301A10-Ab2 VH
caggttcagctgcagcagtctggagctgaactggcgaggcctggggcttcagtgaagctgtcctgcaaggcttctggctacaccttcacaagctatggtatcagctgggtgaagcagagaactggacagggccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagtgagagctactatggtgactacgactggtacttcgatgtctggggcactgggaccacggtcaccgtctcctca

SEQ ID NO: 729 - DNA, 336 bp, Artificial Sequence
  ACI-8032-6301A10-Ab2 VL
gatgttttgatgacccaaactccactctccctgcctgtcagtcttggagatcaagcctccatctcttgcagatctagtcacagcattgaacatagtaatggaaacacctatttagaatggtgcctgcagaaaccaggccagcctccaaagttcctgatctacaaagtttccaaccgattttctggggtcccagacaggttcagtggcagtggatcagggacagatttcacactcaggatcagtagagtggaggctgaggatctgggagtttattactgctttcaaggttcacatgttcctccgacgttcggtggaggcaccaagctggaaatcaaa

SEQ ID NO: 730 - protein, 122 aa, Artificial Sequence
  ACI-8032-6301C8-Ab2 VH
QVQLQQSGAELARPGTSVKLSCKASGYTFTTYGISWVKQRTGQGLEWIGEIYPRSGNTYYNEKFKGKATLTADKPSSTAYMELRSLTSEDSAVYFCASESYYGDYAWYFDVWGTGTTVTVSS

SEQ ID NO: 731 - protein, 5 aa, Artificial Sequence
  ACI-8032-6301C8-Ab2 VH CDR1
TYGIS

SEQ ID NO: 732 - skipped

SEQ ID NO: 733 - protein, 13 aa, Artificial Sequence
  ACI-8032-6301C8-Ab2 VH CDR3
ESYYGDYAWYFDV

SEQ ID NO: 734 - protein, 112 aa, Artificial Sequence
  ACI-8032-6301C8-Ab2 VL
DVLMTQIPLSLPVSLGDQASISCRSSQSIVHSNGNTYLEWHLQKPGQSPKLLIYEVSNRFSGVPDRFSGSGSGTDFTLNISRVEAEDLGVYYCFQGSHVPPTFGGGTKLEIK

SEQ ID NO: 735 - protein, 16 aa, Artificial Sequence
  ACI-8032-6301C8-Ab2 VL CDR1
RSSQSIVHSNGNTYLE

SEQ ID NO: 736 - protein, 7 aa, Artificial Sequence
  ACI-8032-6301C8-Ab2 VL CDR2
EVSNRFS

SEQ ID NO: 737 - skipped

SEQ ID NO: 738 - DNA, 366 bp, Artificial Sequence
  ACI-8032-6301C8-Ab2 VH
caggttcagctgcagcagtctggagctgaactggcgaggcctgggacttcagtgaagctgtcctgcaaggcttctggctacaccttcacaacctatggtataagctgggtgaagcagagaactggacagggccttgagtggattggagagatttatcctagaagtggtaatacttactacaatgagaaattcaagggcaaggccacactgactgcagacaagccctccagcacagcgtacatggagctccgcagcctgacatctgaggactctgcggtctatttctgtgcaagtgagagttactatggtgactacgcctggtacttcgatgtctggggcacagggaccacggtcaccgtctcctca

SEQ ID NO: 739 - DNA, 336 bp, Artificial Sequence
  ACI-8032-6301C8-Ab2 VL
gatgttttgatgacccaaattccactctccctgcctgtcagtcttggagatcaagcctccatctcttgcagatctagtcagagcattgtacatagtaatggaaacacctatttagaatggcacctgcagaaaccaggccagtctccaaagctcctgatctacgaagtttccaaccgattttctggggtcccagacaggttcagtggcagtggatcagggacagatttcacactcaatatcagcagagtggaggctgaggatctgggagtttattactgctttcaaggttcacatgttcctccgacgttcggtggaggcaccaagctggaaatcaaa

SEQ ID NO: 740 - protein, 122 aa, Artificial Sequence
  ACI-8032-6301G2-Ab2 VH
EVQLQQSGPELVKPGASVKISCKASGYTFTDYFMNWVKQSHGKSLEWIGDINPNIGVTNYNPKFKDRATLTVDKSSSSAYMELRSLTSEDSTVYYCARGGGSNPLYYAMDYWGQGTSVTVSS

SEQ ID NO: 741 - skipped

SEQ ID NO: 742 - protein, 17 aa, Artificial Sequence
  ACI-8032-6301G2-Ab2 VH CDR2
DINPNIGVTNYNPKFKD

SEQ ID NO: 743 - protein, 13 aa, Artificial Sequence
  ACI-8032-6301G2-Ab2 VH CDR3
GGGSNPLYYAMDY

SEQ ID NO: 744 - protein, 112 aa, Artificial Sequence
  ACI-8032-6301G2-Ab2 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYFCKQSYDLWTFGGGTKLEIK

SEQ ID NO: 745-747 - skipped (3 entries)

SEQ ID NO: 748 - DNA, 366 bp, Artificial Sequence
  ACI-8032-6301G2-Ab2 VH
gaggtccaactgcaacaatctggacctgagctggtgaagcctggggcttcagtgaagatatcctgtaaggcttctggatacacgttcactgactacttcatgaactgggtgaagcagagccatggaaagagccttgagtggattggagatattaatcctaacattggtgttactaactacaacccgaagttcaaggacagggccacattgactgtagacaagtcctccagctcagcctacatggagctccgcagcctgacatctgaggactctacagtctattactgtgcaagagggggagggagtaatcccctttactatgctatggactactggggtcaaggaacctcagtcaccgtctcctca

SEQ ID NO: 749 - DNA, 336 bp, Artificial Sequence
  ACI-8032-6301G2-Ab2 VL
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttatttctgcaagcaatcttatgatctgtggacgttcggtggaggcaccaagctggaaatcaaa

SEQ ID NO: 750 - protein, 122 aa, Artificial Sequence
  ACI-8032-6304F3-Ab1 VH
QVQLQQSGAELARPGASVKLSCKASGYTFTSYGVSWVKQRTGQGLEWIGEIYPRSGNTYYNEKFKGKATLTADKSSSTAYMELRSLTSEDSAVYFCASESYYGDYDWYFDVWGTGTTVTVSS

SEQ ID NO: 751 - protein, 5 aa, Artificial Sequence
  ACI-8032-6304F3-Ab1 VH CDR1
SYGVS

SEQ ID NO: 752-753 - skipped (2 entries)

SEQ ID NO: 754 - protein, 112 aa, Artificial Sequence
  ACI-8032-6304F3-Ab1 VL
DVLMTQIPLSLPVSPGDQVSISCRSSQSIVHSNGNTYLEWYLQKPGQSPKPLIYKVSNRFSGVPDRFSGSGSGTDFTLKISRVEAEDLGVYYCFQGSHVPPTFGGGTKLEIK

SEQ ID NO: 755-757 - skipped (3 entries)

SEQ ID NO: 758 - DNA, 366 bp, Artificial Sequence
  ACI-8032-6304F3-Ab1 VH
caggttcagctgcagcagtctggagctgagctggcgagacctggggcttcagtgaagctgtcctgcaaggcttctggctacaccttcacaagctatggtgtaagctgggtgaagcagagaactggacagggccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagtgagagctactatggtgactacgactggtatttcgatgtctggggcacagggaccacggtcaccgtctcctca

SEQ ID NO: 759 - DNA, 336 bp, Artificial Sequence
  ACI-8032-6304F3-Ab1 VL
gatgttttgatgacccaaattccactctccctgcctgtcagtcctggagatcaagtttccatctcttgcagatctagtcagagcattgtacatagtaatggaaacacctatttagaatggtacctgcagaaaccaggccagtctccaaagcccctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 760 - protein, 122 aa, Artificial Sequence
  ACI-8032-6307F1-Ab2 VH
QVQLQQSGAELARPGASVKLSCKASGYTFTSYGMSWVKQRTGQGLEWIGEIYPRSGNTYYNEKFKGKATLTSDKSSSTSYMELRSLTSEDSAVYFCASESYYGDYAWYFDVWGTGTTVTVSS

SEQ ID NO: 761 - protein, 5 aa, Artificial Sequence
  ACI-8032-6307F1-Ab2 VH CDR1
SYGMS

SEQ ID NO: 762-763 - skipped (2 entries)

SEQ ID NO: 764 - protein, 112 aa, Artificial Sequence
  ACI-8032-6307F1-Ab2 VL
DVLMTQTPLSLPVSLGDQASISCRSSQSVVHSNGITYLEWYLQKPGQSPKLLIYKVSNRFSGVPDRFSGSGSGTYFTLKISRVEAEDLGVYYCFQGSHVPPTFGGGTKLEIK

SEQ ID NO: 765 - protein, 16 aa, Artificial Sequence
  ACI-8032-6307F1-Ab2 VL CDR1
RSSQSVVHSNGITYLE

SEQ ID NO: 766-767 - skipped (2 entries)

SEQ ID NO: 768 - DNA, 366 bp, Artificial Sequence
  ACI-8032-6307F1-Ab2 VH
caggttcagctgcagcagtctggagctgagctggcgaggcctggggcttcagtgaagctgtcctgcaaggcttctggctacaccttcacaagctatggtatgagttgggtgaaacagagaactggacagggccttgagtggattggagagatttatcctagaagtggtaatacttactacaatgagaagttcaagggcaaggccacactgacttcagacaaatcctccagcacatcatacatggagctccgcagcctgacatctgaggactctgcggtctatttctgtgcaagtgagagctactatggtgactacgcctggtacttcgatgtctggggcacagggaccacggtcaccgtctcctca

SEQ ID NO: 769 - DNA, 336 bp, Artificial Sequence
  ACI-8032-6307F1-Ab2 VL
gatgttttgatgacccaaactccactctccctgcctgtcagtcttggagatcaagcctccatctcttgcagatctagtcagagcgttgtacatagtaatggaatcacctatttagaatggtacctgcagaaaccaggccagtctccaaagctcctgatctacaaagtttccaaccgattttctggggtcccagacaggttcagtggcagtggatcagggacatatttcacactcaagatcagcagagtggaggctgaggatcttggagtttattactgctttcaaggttcacatgttcctccgacgttcggtggaggcaccaagctggaaatcaaa

SEQ ID NO: 770 - protein, 123 aa, Artificial Sequence
  ACI-8032-6313G2-Ab1 VH
EVQLQQSGPELVKPGASVKISCKASGYTFTDYYMNWVKQSHGKSLEWIGDIDPNNGVTSYSQKFKGKATLTVDKSSSTAYMELRSLTSEDSAVYYCARTGYYGTSLYYAMDYWGQGTSVTVSS

SEQ ID NO: 771 - protein, 5 aa, Artificial Sequence
  ACI-8032-6313G2-Ab1 VH CDR1
DYYMN

SEQ ID NO: 772 - protein, 17 aa, Artificial Sequence
  ACI-8032-6313G2-Ab1 VH CDR2
DIDPNNGVTSYSQKFKG

SEQ ID NO: 773 - protein, 14 aa, Artificial Sequence
  ACI-8032-6313G2-Ab1 VH CDR3
TGYYGTSLYYAMDY

SEQ ID NO: 774 - protein, 112 aa, Artificial Sequence
  ACI-8032-6313G2-Ab1 VL
DIVMSQSPSSLAVSAGEKVTMNCKSSQSLLNSRTRKNYLAWYQQKPGQSPKLLIYWASTRESGVPDRFIGSGSGTGFTLTISSVQAEDLAVYYCKQSYDLWTFGGGTKLEIK

SEQ ID NO: 775-777 - skipped (3 entries)

SEQ ID NO: 778 - DNA, 369 bp, Artificial Sequence
  ACI-8032-6313G2-Ab1 VH
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattgatcctaacaatggtgttactagttacagccagaagttcaagggcaaggccacattgactgtagacaagtcctccagcacagcctacatggagctccgcagcctgacatctgaagactctgcagtctattactgtgcaaggacggggtactacggtacttctctatactatgctatggactactggggtcaaggaacctcagtcaccgtctcctca

SEQ ID NO: 779 - DNA, 336 bp, Artificial Sequence
  ACI-8032-6313G2-Ab1 VL
gacattgtgatgtcacagtcgccatcctccctggctgtgtcagcaggagagaaggtcactatgaactgcaaatccagtcagagtctgctcaacagtagaacccgaaagaactacttggcttggtaccagcagaaaccagggcagtctccaaaactactgatctactgggcatccactagggaatctggggtccctgatcgcttcataggcagtggatctgggacaggtttcactctcaccattagcagtgtgcaggctgaagacctggcagtttattactgcaagcaatcttatgatctgtggacgttcggtggaggcaccaagctggaaatcaaa

SEQ ID NO: 780-783 - skipped (4 entries)

SEQ ID NO: 784 - protein, 112 aa, Artificial Sequence
  ACI-8032-6314A3-Ab3 VL
DVLMTQIPLSLPVSPGDQVFISCRSSQSIVHSNGNTYLEWYLQKPGQSPKPLIYKVSNRFSGVPDRFSGSGSGTDFTLKISRVEAEDLGVYYCFQGSHVPPTFGGGTKLEIK

SEQ ID NO: 785-788 - skipped (4 entries)

SEQ ID NO: 789 - DNA, 336 bp, Artificial Sequence
  ACI-8032-6314A3-Ab3 VL
gatgttttgatgacccaaattccactctccctgcctgtcagtcctggagatcaagttttcatctcttgcagatctagtcagagcattgtacatagtaatggaaacacctatttagaatggtacctgcagaaaccaggccagtctccaaagcccctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 790 - protein, 116 aa, Artificial Sequence
  ACI-8033-6401F2-Ab1 VH
QVQLQQPGAELVKPGASVKMSCKASGYTFTNYWITWVKQRPGQGLEWIGDIYPGNGDVDYSEIFKNKAKMTVDTSSTTAYMQLSSLTSEDSAVYHCVLGQTSFGHWGQGTLVTVSA

SEQ ID NO: 791 - protein, 5 aa, Artificial Sequence
  ACI-8033-6401F2-Ab1 VH CDR1
NYWIT

SEQ ID NO: 792 - protein, 17 aa, Artificial Sequence
  ACI-8033-6401F2-Ab1 VH CDR2
DIYPGNGDVDYSEIFKN

SEQ ID NO: 793 - protein, 7 aa, Artificial Sequence
  ACI-8033-6401F2-Ab1 VH CDR3
GQTSFGH

SEQ ID NO: 794 - protein, 112 aa, Artificial Sequence
  ACI-8033-6401F2-Ab1 VL
DVLMTQTPLSLPVSLGDQASISCRSSQSVVHVNGNTYLDWYLQKPGQSPKLLIYKVSNRFSGVPDRFSGSGSGTDFTLRITRVEAEDLGIYYCFQGSHVPRTFGGGTKLEIK

SEQ ID NO: 795 - protein, 16 aa, Artificial Sequence
  ACI-8033-6401F2-Ab1 VL CDR1
RSSQSVVHVNGNTYLD

SEQ ID NO: 796 - skipped

SEQ ID NO: 797 - protein, 9 aa, Artificial Sequence
  ACI-8033-6401F2-Ab1 VL CDR3
FQGSHVPRT

SEQ ID NO: 798 - DNA, 348 bp, Artificial Sequence
  ACI-8033-6401F2-Ab1 VH
caggtccaactgcagcagcctggggctgagcttgtgaagcctggggcttcagtgaagatgtcctgcaaggcttctggctacaccttcaccaactactggattacctgggtaaaacagaggcctggacaaggccttgagtggattggggatatttatcctggtaatggtgatgttgactacagtgagatcttcaagaataaggccaaaatgactgtagacacatcctccaccacagcctacatgcagctcagcagcctgacatctgaggactctgcggtctatcactgtgtcttgggtcagacttcctttggtcattggggccaagggactctggtcactgtctctgca

SEQ ID NO: 799 - DNA, 336 bp, Artificial Sequence
  ACI-8033-6401F2-Ab1 VL
gatgttttgatgacccaaactccactctccctgcctgtcagtcttggagatcaagcctccatctcttgcagatctagtcagagtgttgtgcatgttaatggaaacacctatttagattggtacctgcagaaaccaggccagtctccaaagctcctgatctacaaagtttccaaccgattttctggggtcccagacaggttcagtggcagtggatcagggacagatttcacgctcaggatcaccagagtggaggctgaggatctgggaatttattactgttttcaaggttcacatgttcctcggacgttcggtggaggcaccaagctggaaatcaaa

SEQ ID NO: 800 - protein, 119 aa, Artificial Sequence
  ACI-8033-6402E2-Ab2 VH
EVQLQHSGPDLVKPGASVKISCKASGYTFTDYYMNWVKQSHGKSLEWIGDINPNNGDTTYNQKFKGRATLTVDKSSSTAYMELRSLTSEDSAVYYCARSGSNYYAMDSWGQGTSVTVSS

SEQ ID NO: 801 - skipped

SEQ ID NO: 802 - protein, 17 aa, Artificial Sequence
  ACI-8033-6402E2-Ab2 VH CDR2
DINPNNGDTTYNQKFKG

SEQ ID NO: 803 - protein, 10 aa, Artificial Sequence
  ACI-8033-6402E2-Ab2 VH CDR3
SGSNYYAMDS

SEQ ID NO: 804 - protein, 112 aa, Artificial Sequence
  ACI-8033-6402E2-Ab2 VL
DIVMSQSPSSLAVSAGERVTMSCKSSQSLFNSRTRKNYLAWYQQKPGQSPKLLIYWASTRESGVPDRFTGSGSGTDFSLNISSVQAEDLAVYYCKQSYDLWTFGGGTKLEIK

SEQ ID NO: 805 - protein, 17 aa, Artificial Sequence
  ACI-8033-6402E2-Ab2 VL CDR1
KSSQSLFNSRTRKNYLA

SEQ ID NO: 806-807 - skipped (2 entries)

SEQ ID NO: 808 - DNA, 357 bp, Artificial Sequence
  ACI-8033-6402E2-Ab2 VH
gaggtccagctgcaacattctggacctgacctggtgaagcctggggcttcagtgaagatatcctgtaaggcttctggatacacgttcactgactactacatgaactgggtgaaacagagccatggaaagagccttgagtggattggagatattaatcctaacaatggtgatactacctacaaccagaagttcaagggcagggccacattgactgtagacaagtcctccagcacagcctacatggagctccgcagcctgacatctgaggactctgcagtctattactgtgcaagatcggggagtaattactatgctatggactcctggggtcaaggaacctcagtcaccgtctcctca

SEQ ID NO: 809 - DNA, 336 bp, Artificial Sequence
  ACI-8033-6402E2-Ab2 VL
gacattgtgatgtcacagtctccatcctccctggctgtgtcagcaggagagagggtcactatgagctgcaaatccagtcagagtctgttcaacagtagaacccgaaagaactacttggcttggtaccagcagaaaccagggcagtctcctaaactgctgatctactgggcatccactagggaatctggggtccctgatcgcttcacaggcagtggatctgggacagatttcagtctcaacatcagcagtgtgcaggctgaagacctggcagtttattactgcaagcaatcttatgatctgtggacgttcggtggaggcaccaagctggaaatcaaa

SEQ ID NO: 810 - protein, 113 aa, Artificial Sequence
  ACI-8033-6402E10-Ab1 VH
DVQLQESGPGLVKPVQSLSLTCSVTGYSITSGYYWNWIRQFPGNKLEWMGYISSDGSNNYNPSLKNRISISRDTSKNQFFLKLDSVTTEDTATYYCVRADNYWGQGTTLTVSS

SEQ ID NO: 811 - protein, 6 aa, Artificial Sequence
  ACI-8033-6402E10-Ab1 VH CDR1
SGYYWN

SEQ ID NO: 812 - protein, 16 aa, Artificial Sequence
  ACI-8033-6402E10-Ab1 VH CDR2
YISSDGSNNYNPSLKN

SEQ ID NO: 813 - protein, 4 aa, Artificial Sequence
  ACI-8033-6402E10-Ab1 VH CDR3
ADNY

SEQ ID NO: 814 - protein, 112 aa, Artificial Sequence
  ACI-8033-6402E10-Ab1 VL
DVVMTQTPLTLSVTIGQPASISCKSSQSLLDSDGETYLNWLLQRPGQSPKRLIYLVSKVDSGVPDRFTGRGSGTDFTLKISRVEAEDLGVYYCWQGTHFPQTFGGGTKLEIK

SEQ ID NO: 815 - protein, 16 aa, Artificial Sequence
  ACI-8033-6402E10-Ab1 VL CDR1
KSSQSLLDSDGETYLN

SEQ ID NO: 816 - skipped

SEQ ID NO: 817 - protein, 9 aa, Artificial Sequence
  ACI-8033-6402E10-Ab1 VL CDR3
WQGTHFPQT

SEQ ID NO: 818 - DNA, 339 bp, Artificial Sequence
  ACI-8033-6402E10-Ab1 VH
gatgtacagcttcaggagtcaggacctggcctcgtgaaacctgttcagtctttgtctctcacctgctctgtcactggctactccatcaccagtggttattactggaactggatccggcagtttccaggaaacaaactggaatggatgggctacataagctccgatggtagcaataactacaacccatctctcaaaaatcgaatctccatctctcgtgacacatctaagaaccagtttttcctgaagttggattctgtgactactgaggacacagccacatattactgtgtaagagcggacaactactggggccaaggcaccactctcacagtctcctcg

SEQ ID NO: 819 - DNA, 336 bp, Artificial Sequence
  ACI-8033-6402E10-Ab1 VL
gatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctatctggtgtctaaagtggactctggagtccctgacaggttcactggcagaggatcagggacagatttcacactgaaaatcagcagagtggaggctgaggatttgggagtttattattgctggcaaggcacacattttcctcagacgttcggtggaggcaccaagctggaaatcaag

SEQ ID NO: 820 - protein, 115 aa, Artificial Sequence
  ACI-8033-6403A4-Ab1 VH
EVQLVESGGGLVQPKGSLKLSCAASGFSFNTYAMNWVRQAPGKGLEWVARIRSKSNNYTTYYADSVKDRFTISRDDSESMLYLQMNNLKTEDTAMYYCVRTMDYWGQGTSVTVSS

SEQ ID NO: 821 - protein, 5 aa, Artificial Sequence
  ACI-8033-6403A4-Ab1 VH CDR1
TYAMN

SEQ ID NO: 822 - protein, 19 aa, Artificial Sequence
  ACI-8033-6403A4-Ab1 VH CDR2
RIRSKSNNYTTYYADSVKD

SEQ ID NO: 823 - protein, 4 aa, Artificial Sequence
  ACI-8033-6403A4-Ab1 VH CDR3
TMDY

SEQ ID NO: 824 - protein, 105 aa, Artificial Sequence
  ACI-8033-6403A4-Ab1 VL
QIVLTQSPAIMSASLGEEITLTCSASSSISYMHWYQQKSGTSPKLLIHSTFNLASGVPSRFSGSGSGTFYSLTISSVEAEDAADYYCHQWSSYYTFGGGTKLEIK

SEQ ID NO: 825 - protein, 10 aa, Artificial Sequence
  ACI-8033-6403A4-Ab1 VL CDR1
SASSSISYMH

SEQ ID NO: 826 - protein, 7 aa, Artificial Sequence
  ACI-8033-6403A4-Ab1 VL CDR2
STFNLAS

SEQ ID NO: 827 - protein, 8 aa, Artificial Sequence
  ACI-8033-6403A4-Ab1 VL CDR3
HQWSSYYT

SEQ ID NO: 828 - DNA, 345 bp, Artificial Sequence
  ACI-8033-6403A4-Ab1 VH
gaggtgcagcttgttgagtctggtggaggattggtgcagcctaaagggtcattgaaactctcatgtgcagcctctggattcagtttcaatacctacgccatgaactgggtccgccaggctccaggaaagggtttggaatgggttgctcgcataagaagtaaaagtaataattatacaacatattatgccgattcagtgaaagacagattcaccatctccagagatgattcagaaagcatgctctatctgcaaatgaacaacttgaaaactgaggacacagccatgtattactgtgtgagaactatggactactggggtcaaggaacctcagtcaccgtctcctca

SEQ ID NO: 829 - DNA, 315 bp, Artificial Sequence
  ACI-8033-6403A4-Ab1 VL
caaattgttctcacccagtctccagcaatcatgtctgcatctctaggggaggagatcaccctaacctgcagtgccagttcgagtataagttacatgcactggtaccagcagaagtcaggcacttctcccaaactcttgattcatagcacattcaacctggcttctggagtcccttctcgcttcagtggcagtgggtctgggaccttttattctctcacaatcagcagtgtggaggctgaagatgctgccgattattactgccatcagtggagtagttattacacgttcggaggggggaccaagctggaaataaaa

SEQ ID NO: 830 - protein, 113 aa, Artificial Sequence
  ACI-8033-6403E11-Ab2 VH
DVQLQESGPGLVKPSQSLSLTCSVTGYSITSAYYWNWIRQFPGNKLEWMGYISYDGSNNYIPSLKNRISISRDTSKNQFFLKLNSVTTEDTATYYCIRGDVNWGQGTTLTVSS

SEQ ID NO: 831 - protein, 6 aa, Artificial Sequence
  ACI-8033-6403E11-Ab2 VH CDR1
SAYYWN

SEQ ID NO: 832 - protein, 16 aa, Artificial Sequence
  ACI-8033-6403E11-Ab2 VH CDR2
YISYDGSNNYIPSLKN

SEQ ID NO: 833 - protein, 4 aa, Artificial Sequence
  ACI-8033-6403E11-Ab2 VH CDR3
GDVN

SEQ ID NO: 834 - protein, 112 aa, Artificial Sequence
  ACI-8033-6403E11-Ab2 VL
DVVMTQTPLTLSVTIGQPASISCKSSQSLLDGDGETYLNWLLQRPGQSPKRLIYLVSKLDSGVPDRFTGSGSGTDFTLKISRVEAEDLGVYYCWQGTHFPQTFGGGTKLEIK

SEQ ID NO: 835 - protein, 16 aa, Artificial Sequence
  ACI-8033-6403E11-Ab2 VL CDR1
KSSQSLLDGDGETYLN

SEQ ID NO: 836 - protein, 7 aa, Artificial Sequence
  ACI-8033-6403E11-Ab2 VL CDR2
LVSKLDS

SEQ ID NO: 837 - protein, 9 aa, Artificial Sequence
  ACI-8033-6403E11-Ab2 VL CDR3
WQGTHFPQT

SEQ ID NO: 838 - DNA, 339 bp, Artificial Sequence
  ACI-8033-6403E11-Ab2 VH
gatgtacagcttcaggagtcaggacctggcctcgtgaaaccttctcagtctctgtctctcacctgctctgtcactggctactccatcaccagtgcttattactggaactggatccggcagtttccaggaaacaaactggaatggatgggctacataagctacgatggtagcaataactacatcccttctctcaaaaatcgaatctccatcagtcgtgacacatctaagaaccagtttttcctgaagttgaattctgtgactactgaggacacagccacatattactgtataagaggggatgttaactggggccaaggcaccactctcacagtctcctca

SEQ ID NO: 839 - DNA, 336 bp, Artificial Sequence
  ACI-8033-6403E11-Ab2 VL
gatgttgtgatgacccagactccactcactttgtcggtcaccattggacaaccagcctccatctcttgcaagtcaagtcagagcctcttagatggtgatggagagacatatttgaattggttgttacagaggccaggccagtctccaaagcgcctaatctatctggtatctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 840 - protein, 119 aa, Artificial Sequence
  ACI-7067-4813-R4A-G7-rec1 VH
EVQLEESGGGLVHPKGSLKLSCAASGFTFNSYAMHWVRQAPGKGLEWVARIRSKRSNYATYYADSVKDRFTISRDDSQSMVYLEMNNLEAEDTATYYCVRGGREAMDFWGQGTSVTVSS

SEQ ID NO: 841 - protein, 5 aa, Artificial Sequence
  ACI-7067-4813-R4A-G7-rec1 CDR1
SYAMH

SEQ ID NO: 842 - protein, 19 aa, Artificial Sequence
  ACI-7067-4813-R4A-G7-rec1 VH CDR2
RIRSKRSNYATYYADSVKD

SEQ ID NO: 843 - protein, 8 aa, Artificial Sequence
  ACI-7067-4813-R4A-G7-rec1 VH CDR3
GGREAMDF

SEQ ID NO: 844 - protein, 109 aa, Artificial Sequence
  ACI-7067-4813-R4A-G7-rec1 VL
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 845 - protein, 14 aa, Artificial Sequence
  ACI-7067-4813-R4A-G7-rec1 VL CDR1
RSSTGAVTTSNYAN

SEQ ID NO: 846 - protein, 7 aa, Artificial Sequence
  ACI-7067-4813-R4A-G7-rec1 VL CDR2
GTNNRAP

SEQ ID NO: 847 - protein, 7 aa, Artificial Sequence
  ACI-7067-4813-R4A-G7-rec1 VL CDR3
ALWYSNH

SEQ ID NO: 848 - DNA, 357 bp, Artificial Sequence
  ACI-7067-4813-R4A-G7-rec1 VH
gaggtgcagctggaggagtctggtggaggattggtgcaccctaaaggatcattgaaactctcatgtgccgcctctggtttcaccttcaattcctatgccatgcactgggtccgccaggctccaggaaagggtttggaatgggttgctcgcataagaagtaaaagaagtaattatgcaacatattatgccgattcagtgaaagacagattcaccatctccagagatgattcacaaagcatggtctatctggaaatgaacaacctggaagctgaggacacagccacgtattactgtgtgagagggggacgagaggctatggacttctggggtcaaggaacctcagtcaccgtctcctca

SEQ ID NO: 849 - DNA, 327 bp, Artificial Sequence
  ACI-7067-4813-R4A-G7-rec1 VL
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 850 - protein, 60 aa, Unknown
  Binding Epitope from an organism of the kingdom Mammalia
MDVFMKGLSKAKEGVVAAAEKTKQGVAEAAGKTKEGVLYVGSKTKEGVVHGVATVAEKTK

SEQ ID NO: 851 - protein, 35 aa, Unknown
  Binding Epitope from an organism of the kingdom Mammalia
EQVTNVGGAVVTGVTAVAQKTVEGAGSIAAATGFV

SEQ ID NO: 852 - protein, 330 aa, Artificial Sequence
  Constant Region A_HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 853 - protein, 330 aa, Artificial Sequence
  Constant Region B_HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 854 - protein, 105 aa, Artificial Sequence
  Constant Region C_LC
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 855 - protein, 330 aa, Artificial Sequence
  Constant Region D_HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 856 - protein, 334 aa, Artificial Sequence
  Constant Region E_HC
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 857 - protein, 330 aa, Artificial Sequence
  Constant Region F_HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 858 - protein, 358 aa, Artificial Sequence
  Constant Region G_HC
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 859 - protein, 95 aa, Artificial Sequence
  Constant Region H_LC
PDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 860 - protein, 330 aa, Artificial Sequence
  Constant Region I_HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 861 - protein, 330 aa, Artificial Sequence
  Constant Region J_HC
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 862 - protein, 107 aa, Artificial Sequence
  Constant Region K_LC
RTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 863 - protein, 120 aa, Unknown
  Binding Epitope from an organism of the kingdom Mammalia
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDP